(19) United States
(12) Patent Application Publication
(10) Pub. No.: US 2026/0118360 A1
(43) Pub. Date: Apr. 30, 2026

(54) CHEMICAL COMPOSITIONS AND METHODS OF USE

(71) Applicant: Liquid Biopsy Research LLC, Charlestown (KN)

(72) Inventors: Irvin Mark MODLIN, Woodbridge, CT (US); Mark KIDD, New Haven, CT (US); Ignat DROZDOV, Stratford Upon Avon (GB)

(21) Appl. No.: 19/238,168

(22) Filed: Jun. 13, 2025

Related U.S. Application Data
(63) Continuation of application No. 17/501,168, filed on Oct. 14, 2021, now Pat. No. 12,366,575.
Application No. 17/501,168 is a continuation of application No. 16/039,041, filed on Jul. 18, 2018, now abandoned.
(60) Provisional application No. 62/535,419, filed on Jul. 21, 2017.

(51) Int. Cl. G01N 33/57505; C12Q 1/6886; G01N 33/575; G01N 33/68; G16H 50/30

(52) U.S. Cl. CPC G01N 33/57505; C12Q 1/6886; G01N 33/6818; G01N 33/6857; G16H 50/30; C12Q 2600/112; C12Q 2600/118; C12Q 2600/158; G01N 33/57585; G01N 2800/52; G01N 2800/54; G01N 2800/60; G01N 2800/7028

(57) ABSTRACT

The present invention is directed to methods for detecting a plasma cell dyscrasia like myeloma or MGUS, methods for determining whether a plasma cell dyscrasiais stable or progressive, methods for determining a risk for disease relapse, and methods for determining a response by a subject having a plasma cell dyscrasia to a therapy.

### CROSS-REFERENCE TO RELATED APPLICATIONS

This application is a Continuation of U.S. patent application Ser. No. 17/501,168, filed on Oct. 14, 2021, which is a continuation of U.S. patent application Ser. No. 16/039,041, filed on Jul. 18, 2018, which claims the benefit of and priority to U.S. Provisional Application No. 62/535,419, filed Jul. 21, 2017. The contents of each of the aforementioned patent applications are hereby incorporated by reference in their entireties for all purposes.

### INCORPORATION BY REFERENCE OF SEQUENCE LISTING

The contents of the electronic sequence listing (LBIO_002_C02US_SeqList_ST26.xml; Size: 260,359 bytes; and Date of Creation: Jun. 13, 2025) are herein incorporated by reference in their entirety.

### FIELD OF THE INVENTION

The present invention relates to plasma cell dyscrasia detection.

### BACKGROUND OF THE INVENTION

Multiple myeloma is an incurable hematological malignancy of end-stage B-lineage or plasma cells. This clonal plasma cell malignancy accounts for ˜2% of all cancer cases and approximately 10% of hematologic malignancies. It is the third most common B cell malignancy after diffuse B-cell lymphoma and chronic lymphocytic leukemia. The incidence is estimated at (˜1/100,000 incidence in the US). In 2017, more than 12,500 deaths resulted from the disease. Patients older than 65 years account for >80% of those diagnosed; there is a twofold increased incidence in blacks compared with whites. There has been a significant rise in the incidence of the disease over the last fifty years. Moreover, overall survival has improved in the last two decades from a median of ˜3.5 years to ˜6 years with an average 5-year survival of ˜50%. This is attributed to innovative treatments and the widespread use of proteasome inhibitors and immunomodulatory drugs but many patients exhibit low progression-free survival rates and have a poor overall survival. This reflects tumor heterogeneity, drug resistance and the immunosuppressive nature of the tumor within the osteo-microenvironment.

Multiple myelomas typically progress from asymptomatic precursor stages (monoclonal gammopathy of undetermined significance: MGUS) and smoldering multiple myeloma (SMM) to frank disease. Some exhibit rapid progression to MM, whilst others have life-long indolent disease. The heterogeneity and genomic complexity of the disease, and particularly intraclonal heterogeneity, underpins the heterogeneous evolution of disease, responses to therapy as well as progression after “successful” treatment.

Multiple myelomas exhibit notable inter- and intrapatient heterogeneity. This is reflected in copy number variations including hyperdiploidy and focal or Chromosome (Chr) arm gains or amplifications e.g., Chr 1q or loss e.g., Chr 17p, and translocations involving the immunoglobulin heavy chain locus on chromosome 14. These are all hallmarks of multiple myeloma pathogenesis. Hyperdiploidy and chromosomal translocations are the most common genetic aberrations and both are considered primary events. Secondary events that are associated with disease progression include activating translocations e.g., in the MYC oncogene, but these occur in subsets of patients. For example, a MYC translocations either alone, or in conjunction with Chr1q amplification identifies a poor prognostic subtype in hyperdiploid myeloma. While useful, cytogenetic approaches become problematic when two markers predicting opposing outcomes coexist in the same patient. They are also only of modest assistance in indicating appropriate therapeutic strategies and none of them provide predictive information.

Standard blood-based biomarkers e.g., lactate dehydrogenase, albumin or β2 microglobulin (prognostic markers) or serum free light chain (FLC) assays (for disease monitoring) while important in diagnosis and management, are affected by numerous factors including renal failure and other comorbidities or by the cytogenetic profile of an individual disease. They do not measure or encompass the biologic determinants of multiple myeloma.

A number of gene expression assays have been developed from isolated plasma cells. These have involved isolating transformed B-cells from bone marrow aspirates or capturing CD138-positive cells from blood and then undertaking transcriptome-based arrays. These studies have identified gene expression profiles in MM cells that identify high risk patients. GEP70 (70 genes, 30% located at the prognostic Chr1 loci) is a prognostic; CTNI is a multigene centromere amplification-associated prognostic signature; IFM15 includes 15 genes linked to control of the cell cycle (prognostic); HZDC (97 genes—linked to cell death)—prognostic; the PI signature (50 proliferation-associated genes)—prognostic; a signature derived from myeloma cell lines (HMCL—248 genes—“high risk signature”); EMC92 is a 92—gene prognostic signature; CINGEC—a measure of chromosome instability (160 genes—prognostic) and a 17 gene set that may identify patients at risk of early relapse. A number of these signatures have identified the same genes or pathways of activation; all of them require isolation of plasma cells; all require undertaking gene expression array studies. The prognostic utility of these signatures either alone, in combination with other prognostic gene expression signatures or staging systems, have been demonstrated but they function poorly for defining minimal residual disease and do not provide predictive value.

The complex nature of cancer and therapeutic responsiveness comprises a series of “hallmarks”, that include canonical pathways, e.g., RAS and NFκB pathway activation, as well as other features e.g., response to immunotherapy etc. For example, the mutational landscape of newly diagnosed multiple myeloma is dominated by mutations in the RAS (43%) and NFκB (17%) pathways. These are not prognostic but because they can be therapeutically targeted, may be predictive. Identifying prognostic and predictive blood gene signatures without the requirement for isolating plasma cells or the use of bone marrow aspirates therefore is an attractive liquid biopsy approach for this disease.

Recently, such an approach (the NETest) has been developed for tumors with a neuroendocrine phenotype. This blood-based 51-specific mRNA target assay does not require isolation of a specific population of target cells. Gene expression measurements in whole blood correlates with tissue levels and therefore provide direct information about the tumor, its pathophysiology and its state of evolution from stability to progression. This functions as a diagnostic tool and as a surrogate marker of neuroendocrine tumor behavior. Expression of all genes is prognostic; while a subset of genes, those involved in metabolism and the RAS/RAF pathway, predict the response to peptide receptor radiotherapy for this tumor type.

For myelomas, there are no whole blood-derived transcript biomarker panel that functions as a diagnostic or as a prognostic for disease recurrence. A biomarker that can be used to determine minimal residual disease and identify those who will relapse is currently lacking. Moreover, early detection of changes in clonality or the identification of molecular markers of poor prognosis are required.

### SUMMARY OF THE INVENTION

Among other things, disclosed herein is a 32-gene expression tool for plasma cell dyscrasias like MGUS and myeloma. It can have high sensitivity and specificity (>95%) for the detection of a plasma cell dyscrasia and can differentiate minimal residual disease from progressive, active disease. In addition, it can detect patients who are no longer responding to a therapy. Patient clinical status (newly diagnosed, stable/remission or relapsed/refractory) can be predicted with an overall accuracy of >90%.

One aspect of the present disclosure relates to a method for detecting a plasma cell dyscrasia in a subject in need thereof, comprising: (a) determining the expression level of at least 32 biomarkers from a test sample from the subject by contacting the test sample with a plurality of agents specific to detect the expression of the at least 32 biomarkers, wherein the at least 32 biomarkers comprise ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J1, and at least one housekeeping gene; (b) normalizing the expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of the at least one housekeeping gene, thereby obtaining a normalized expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D) 3, and UBE2J1; (c) inputting each normalized expression level into an algorithm to generate a score; (d) comparing the score with a first predetermined cutoff value; and (e) producing a report, wherein the report identifies the presence of a plasma cell dyscrasia in the subject when the score is equal to or greater than the first predetermined cutoff value or determining the absence of a plasma cell dyscrasia in the subject when the score is below the first predetermined cutoff value, wherein the first predetermined cutoff value is 20 on a scale of 0 to 100.

In some embodiments, the method further comprises treating the subject identified as having a plasma cell dyscrasia with drug therapy.

In some embodiments, the first predetermined cutoff value is derived from a plurality of reference samples obtained from subjects free of a neoplastic disease. The reference sample can be blood, serum, plasma, or a non-neoplastic tissue.

Another aspect of the present disclosure relates to a method for determining whether a plasma cell dyscrasia in a subject is stable or progressive, comprising: (a) determining the expression level of at least 32 biomarkers from a test sample from the subject by contacting the test sample with a plurality of agents specific to detect the expression of the at least 32 biomarkers, wherein the at least 32 biomarkers comprise ASXI.1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL.2 (2 splice variants), SMARCD2, SP1 (2 splice variants), SRSF5, TAGAP, TANK, TLE4, TSC22D3, UBE2J1, and at least one housekeeping gene; (b) normalizing the expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of the at least one housekeeping gene, thereby obtaining a normalized expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL.2 (2 splice variants), SMARCD2, SP1 (2 splice variants), SRSF5, TAGAP, TANK, TLE4, TSC22D) 3, and UBE2J1; (c) inputting each normalized expression level into an algorithm to generate a score; (d) comparing the score with a second predetermined cutoff value; and (e) producing a report, wherein the report identifies that the plasma cell dyscrasia is progressive when the score is equal to or greater than the second predetermined cutoff value or identifies that the plasma cell dyscrasia is stable when the score is below the second predetermined cutoff value, wherein the second predetermined cutoff value is 40 on a scale of 0 to 100.

Another aspect of the present disclosure relates to a method for determining a risk of disease relapse in a subject having a plasma cell dyscrasia, comprising: (a) determining the expression level of at least 32 biomarkers from a test sample from the subject after treatment by contacting the test sample with a plurality of agents specific to detect the expression of the at least 32 biomarkers, wherein the at least 32 biomarkers comprise ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J1, and at least one housekeeping gene; (b) normalizing the expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of the at least one housekeeping gene, thereby obtaining a normalized expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1; (c) inputting each normalized expression level into an algorithm to generate a score; (d) comparing the score with a third predetermined cutoff value; and (e) producing a report, wherein the report identifies that the subject has a high risk of disease relapse when the score is equal to or greater than the third predetermined cutoff value or identifies that the subject has a low risk of disease relapse when the score is below the third predetermined cutoff value, wherein the third predetermined cutoff value is 40 on a scale of 0 to 100.

Yet another aspect of the present disclosure relates to a method for determining a response by a subject having a plasma cell dyscrasia to a therapy, comprising: (a) determining a first expression level of at least 31 biomarkers from a first test sample from the subject at a first time point by contacting the first test sample with a plurality of agents specific to detect the expression of the at least 31 biomarkers, wherein the at least 31 biomarkers comprise ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1; (b) determining a second expression level of the at least 31 biomarkers from a second test sample from the subject at a second time point by contacting the second test sample with a plurality of agents specific to detect the expression of the at least 31 biomarkers, wherein the second time point is after the first time point and after the administration of the therapy to the subject; (c) comparing the first expression level with the second expression level; and (d) producing a report, wherein the report identifies that the subject is responsive to the therapy when the second expression level is significantly decreased as compared to the first expression level.

In some embodiments, the first time point is prior to the administration of the therapy to the subject. In some embodiments, the first time point is after the administration of the therapy to the subject. In some embodiments, the therapy comprises a targeted therapy (e.g., a proteasome inhibitor).

In some embodiments of any one of the above aspects, the plasma cell dyscrasia is MGUS or myeloma.

In some embodiments of any one of the above aspects, the at least one housekeeping gene is selected from the group consisting of ALG9, SEPN, YWHAQ, VPS37A, PRRC2B, DOPEY2, NDUFB11, ND4, MRPL19, PSMC4, SF3A1, PUM1, ACTB, GAPD, GUSB, RPLP0, TFRC, MORF4L1, 18S, PPIA, PGK1, RPL13A, B2M, YWHAZ, SDHA, HPRT1, TOX4, and TPT1.

In some embodiments of any one of the above aspects, the method can have a specificity, sensitivity, and/or accuracy of at least 75%, 80%, 85%, 90%, 91%, 92%, 93%, 94%, 95%, 96%, 97%, 98%, or 99%. In some embodiments of any one of the above aspects, the method has a sensitivity of greater than 90%. In some embodiments of any one of the above aspects, the method has a specificity of greater than 90%.

In some embodiments of any one of the above aspects, at least one of the at least 32 biomarkers is RNA, cDNA, or protein. When the biomarker is RNA, the RNA can be reverse transcribed to produce cDNA, and the produced cDNA expression level is detected. In some embodiments of any one of the above aspects, the expression level of the biomarker is detected by forming a complex between the biomarker and a labeled probe or primer. When the biomarker is RNA or cDNA, the RNA or cDNA can be detected by forming a complex between the RNA or cDNA and a labeled nucleic acid probe or primer. When the biomarker is protein, the protein can be detected by forming a complex between the protein and a labeled antibody. The label can be a fluorescent label.

In some embodiments of any one of the above aspects, the test sample is blood, serum, plasma, or a neoplastic tissue. In some embodiments, the reference sample is blood, serum, plasma, or a non-neoplastic tissue.

In some embodiments of any one of the above aspects, the subject in need thereof is a subject diagnosed with a plasma cell dyscrasia, a subject having at least one plasma cell dyscrasia symptom, or a subject have a predisposition or familial history for developing a plasma cell dyscrasia. In some embodiments, the subject is a human.

In some embodiments of any one of the above aspects, the algorithm is XGB, RF, glmnet, cforest, CART, treebag, knn, nnet, SVM-radial, SVM-linear, NB, NNET, mlp, or logistic regression modelling.

### BRIEF DESCRIPTION OF THE DRAWINGS

FIG. 1A and FIG. 1B show a graph showing interactive and functional analyses of the normalized 31 target transcripts (26 genes, 5 splice variants) markers of myeloma. Gene transcripts were significantly functionally linked at an interactome level consistent with a common pattern of regulatory activity. Two trancripts, MCL1 and COPA, were linked with Chromosome 1q, an area known to exhibit amplification in myeloma. At a functional level, the markers identified captured a series of biological processes including angiogenesis, apoptosis, immune responsiveness, phenotype definition, protein processing (secretion), proliferation, RNA processing and survival. A number of the transcripts encoded genes that were either potential drug targets or markers for drug target efficacy.

FIG. 2A and FIG. 2B are graphs showing the receiver operator cuver analysis and metrics for MelanomX (normalized expression of the 26 genes and 5 splice variants) in Test Set I. FIG. 2A is a graph showing that the area under the curve (AUC) was 0.99±0.005 (p<0.0001) for differentiating between myeloma (n=57) and controls (n=23). FIG. 2B is a graph showing that the metrics for MelanomX as a diagnostic has sensitivity>95%, specificity 100%, PPV 100%, NPV 88.5%. The overall accuracy was 96% in Test Set I.

FIG. 3A and FIG. 3B are graphs showing MyelomX scores in MGUS (n=18) versus controls (n=155). FIG. 3A is a graph showing that MGUS patients (n=18) exhibited significantly higher (39±9%, p<0.0001) scores than controls. Control levels were 12±8%. FIG. 3B is a graph showing that the AUC for differentiating MGUS from controls was 0.97±0.01 (p<0.0001).

FIG. 4A and FIG. 4B are graphs showing MyelomX scores in different multiple myeloma sub-groups. FIG. 4A is a graph showing that newly diagnosed patients (n=53) exhibited significantly higher (75±25; p<0.0001) scores than in those with clinically stable disease (n=56; 31±20). Patients with refractory disease (n=26) also presented with higher scores (92±17; p<0.0001 vs. stable disease). FIG. 4B is a graph showing that the AUC for differentiating stable from refractory disease was 0.97±0.03 (p<0.0001).

FIG. 5A, FIG. 5B and FIG. 5C are graphs showing MyelomX scores in Test Set II.

FIG. 5A is a graph showing that the mean MyelomX score in multiple myelomas (n=81) was significantly higher (47±14, p<0.0001) than the control group (n=155; 12±8). FIG. 5B is a graph showing that the AUC was 0.97±0.01 (p<0.0001) for differentiating between myeloma and controls. FIG. 5C is a graph showing that the metrics for MelanomX as a diagnostic (using 20 as a cut-off) has sensitivity 97.5%, specificity 93.6%, PPV 88.8%, NPV 98.6%. The overall accuracy was 94% in Test Set II.

FIG. 6 is a graph showing the effect of therapy on MelanomX. Therapy significantly (p<0.0001) decreased the score from 59±14 (pre-therapy) to 35±12 in 40 patients. Ten patients all with high scores (>40) relapsed at an early time point (within one year).

FIG. 7 is a graph showing the effect of 3 months bortemozib therapy on MelanomX. Therapy significantly (p<0.0001) decreased the score from 64±9 (pre-therapy) to 23±12 in 15 patients who responded to therapy. Eight patients who were refractory exhibited elevated scores (60±9, p=NS vs. pre-treatment).

FIG. 8A and FIG. 8B are a set of graphs showing MyelomX score in 3 different myeloma cell lines. FIG. 8A identifies the cell lines demonstrate elevated expression-MyelomX score ranging from 60 (MM-1R) to 86 (RPMI-8226). FIG. 8B identifies that spiking these cells into blood from a subject that does not have a myeloma or other plasma cell dyscrasia, resulted in detectable gene expression and scores. A minimum of 1 cell/ml of blood could be consistently identified.

FIG. 9A and FIG. 9B are a set of graphs showing the MyelomX score in different FAC-sorted (CD138+) and matched whole blood samples from 3 different multiple myeloma patients. FIG. 9A identifies the scores from FACS samples and whole bloods and identifies the scores are positive and identical. FIG. 9B identifies that gene expression in FACS samples compared to matched blood samples is highly concordant (correlation ˜0.90) consistent with the assay detecting circulating multiple myeloma cells.

### DETAILED DESCRIPTION OF THE INVENTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

Plasma cell dyscrasias (also termed plasma cell disorders and plasma cell proliferative diseases) are a spectrum of progressively more severe monoclonal gammopathies in which a clone or multiple clones of pre-malignant or malignant plasma cells over-produce and secrete into the blood stream a myeloma protein, i.e., an abnormal monoclonal antibody or portion thereof. A plasma cell dyscrasia can develop in different stages. The MGUS stage can be non-IgM MGUS, IgM MGUS, light chain MGUS, or monoclonal gammopathy of renal significance. The smoldering multiple myeloma (SMM) stage can be non-IgM SMM, smoldering Waldenström's macroglobulinemia, or light chain SMM. The malignant sgate can be solitary plasmacytoma, non-secretory multiple myeloma, plasma cell myeloma with concomitant chronic lymphocytic leukemia/monoclonal B-Cell lymphocytosis, Waldenström's macroglobulinemia, multiple myeloma, light chain multiple myeloma, or plasma cell leukemia. In some embodiments, the plasma cell dyscrasia is MGUS. In some embodiments, the plasma cell dyscrasia is myeloma.

The symptoms and signs can vary greatly for patients having myelomas, as many organs can be affected by myelomas. Symptoms can include, but are not limited to, bone pain, anemia, kidney failure, infection, and neurological symptoms (e.g., weakness, confusion, fatigue, headache, visual changes, retinopathy, radicular pain, loss of bowel, bladder control, or carpal tunnel syndrome).

Traditionally, myelomas can be diagnosed through a bood test or urine test. Myeloma cells produce M proteins and beta-2 microglobulin, which can be detected by a blood test. M proteins can also be detected by urine tests. Myelomas can be diagnosed through examination of the bone marrow. Specifically, a sample of bone marrow is removed, and the sample is examined for myeloma cells. Specialized tests, such as fluorescence in situ hybridization (FISH) can analyze myeloma cells to understand their chromosome abnormalities. Tests are also done to measure the rate at which the myeloma cells are dividing. Imaging tests can also be performed to detect bone problems associated with multiple myeloma. Tests may include X-ray, MRI, CT or positron emission tomography (PET).

The present disclosure provides a MyelomX score that can be used for, inter alia, identifying active disease, providing an assessment of treatment responses, predicting risk of relapse, or identifying minimal residual in conjunction with standard clinical assessment and imaging. Measurements of circulating plasma cell dyscrasia transcripts—the MyelomX—can identify plasma cell dyscrasias, and decreases in the MyelomX score correlate with the efficacy of therapeutic interventions such as proteasome inhibitors and immunomodulators. Targeted gene expression profile of RNA can be isolated from the biological samples (e.g., peripheral blood) of patients with plasma cell dyscrasias. This expression profile can be evaluated in an algorithm and converted to an output (prediction).

In one aspect, the present disclosure relates to a method for detecting a plasma cell dyscrasia in a subject in need thereof, comprising: (a) determining the expression level of at least 32 biomarkers from a test sample from the subject by contacting the test sample with a plurality of agents specific to detect the expression of the at least 32 biomarkers, wherein the at least 32 biomarkers comprise ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J1, and at least one housekeeping gene; (b) normalizing the expression level of each of ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of the at least one housekeeping gene, thereby obtaining a normalized expression level of each of ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1; (c) inputting each normalized expression level into an algorithm to generate a score; (d) comparing the score with a first predetermined cutoff value; and (e) producing a report, wherein the report identifies the presence of a plasma cell dyscrasia in the subject when the score is equal to or greater than the first predetermined cutoff value or determining the absence of a plasma cell dyscrasia in the subject when the score is below the first predetermined cutoff value, wherein the first predetermined cutoff value is 20 on a scale of 0 to 100.

The identity of the splice variants for NFKBIZ, NR4A1, PRKAA1, SCYL2, and SP1 can be found at Table 2.

Among the provided methods are those that are able to classify or detect a plasma cell dyscrasia such as MGUS and myeloma. In some embodiments, the provided methods can identify or classify a plasma cell dyscrasia in a human biological sample. In some embodiments, the biological sample is a blood, serum, plasma, or a neoplastic tissue. In some examples, the methods can provide such information with a specificity, sensitivity, and/or accuracy of at least 75%, 80%, 85%, 90%, 91%, 92%, 93%, 94%, 95%, 96%, 97%, 98%, or 99%.

The agents can be any agents for detection of the biomarkers, and typically are isolated polynucleotides or isolated polypeptides or proteins, such as antibodies, for example, those that specifically hybridize to or bind to the at least 32 biomarkers.

The biomarker can be RNA, cDNA, or protein. When the biomarker is RNA, the RNA can be reverse transcribed to produce cDNA (such as by RT-PCR), and the produced cDNA expression level is detected. The expression level of the biomarker can be detected by forming a complex between the biomarker and a labeled probe or primer. When the biomarker is RNA or cDNA, the RNA or cDNA can be detected by forming a complex between the RNA or cDNA and a labeled nucleic acid probe or primer. The complex between the RNA or cDNA and the labeled nucleic acid probe or primer can be a hybridization complex.

When the biomarker is protein, the protein can be detected by forming a complex between the protein and a labeled antibody. The label can be any label, for example a fluorescent label, chemiluminescence label, radioactive label, etc. The protein level can be measured by methods including, but not limited to, immunoprecipitation, ELISA, Western blot analysis, or immunohistochemistry using an agent, e.g., an antibody, that specifically detects the protein encoded by the gene.

In some embodiments, the methods are performed by contacting the test sample with one of the provided agents, more typically with a plurality of the provided agents, for example, a set of polynucleotides that specifically bind to the at least 32 biomarkers. In some embodiments, the set of polynucleotides includes DNA, RNA, cDNA, PNA, genomic DNA, or synthetic oligonucleotides. In some embodiments, the methods include the step of isolating RNA from the test sample prior to detection, such as by RT-PCR, e.g., QPCR. Thus, in some embodiments, detection of the biomarkers, such as expression levels thereof, includes detecting the presence, absence, or amount of RNA. In one example, the RNA is detected by PCR or by hybridization.

In some embodiments, the polynucleotides include sense and antisense primers, such as a pair of primers that is specific to each of the at least 32 biomarkers. In one aspect of this embodiment, the detection of the at least 32 biomarkers is carried out by PCR, typically quantitative or real-time PCR. For example, in one aspect, detection is carried out by producing cDNA from the test sample by reverse transcription; then amplifying the cDNA using the pairs of sense and antisense primers that specifically hybridize to the panel of at least 32 biomarkers, and detecting products of the amplification.

The test sample can be any biological fluid obtained from the subject. Preferably, the test sample is blood, serum, plasma, or a neoplastic tissue. In some embodiments, the test sample is a blood sample.

The first predetermined cutoff value can be derived from a plurality of reference samples obtained from subjects free of a neoplastic disease. Preferably, the reference sample is blood, serum, plasma, or non-neoplastic tissue.

The subject in need thereof can be a subject diagnosed with a plasma cell dyscrasia, a subject having at least one plasma cell dyscrasia symptom, or a subject have a predisposition or familial history for developing a plasma cell dyscrasia. The subject can be any mammal. Preferably, the subject is human. The terms “subject” and “patient” are used interchangeably herein.

In some embodiments, the method can further include determining a mathematically-derived expression level score of the at least 32 biomarkers in the test sample. This is the MyelomX score, which has a scale of 0 to 100. The MyelomX score is the product of a classifier built from a predictive classification algorithm, e.g., XGB, RF, glmnet, cforest, CART, treebag, knn, nnet, SVM-radial, SVM-linear, NB, mlp, or logistic regression modelling. In some embodiments, the predictive classification algorithm used is XGB.

The method can further include treating the subject identified as having a plasma cell dyscrasia with targeted therapy, biological therapy, chemotherapy, corticosteroids, stem cell transplantation, radiation therapy, or a combination thereof. Targeted therapy can include the use of proteasome inhibitors. In some embodiments, the targeted therapy can include bortezomib and/or carfilzomib. Biological therapy can include immunomodulators. In some embodiments, the biological therapy can include thalidomide, lenalidomide, and/or pomalidomide. Chemotherapy can include any known chemotherapeutic drugs. Corticosteroids can be prednisone or dexamethasone.

The present disclosure also provides a method for determining whether a plasma cell dyscrasia in a subject is stable or progressive, comprising: (a) determining the expression level of at least 32 biomarkers from a test sample from the subject by contacting the test sample with a plurality of agents specific to detect the expression of the at least 32 biomarkers, wherein the at least 32 biomarkers comprise ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J1, and at least one housekeeping gene; (b) normalizing the expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of the at least one housekeeping gene, thereby obtaining a normalized expression level of each of ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1; (c) inputting each normalized expression level into an algorithm to generate a score; (d) comparing the score with a second predetermined cutoff value; and (e) producing a report, wherein the report identifies that the plasma cell dyscrasia is progressive when the score is equal to or greater than the second predetermined cutoff value or identifies that the plasma cell dyscrasia is stable when the score is below the second predetermined cutoff value, wherein the second predetermined cutoff value is 40 on a scale of 0 to 100.

The second predetermined cutoff value can be derived from a plurality of reference samples obtained from subjects whose plasma cell dyscrasias are demonstrating disease progression.

The present disclosure also provides a method for determining a risk of disease relapse in a subject having a plasma cell dyscrasia, comprising: (a) determining the expression level of at least 32 biomarkers from a test sample from the subject after treatment by contacting the test sample with a plurality of agents specific to detect the expression of the at least 32 biomarkers, wherein the at least 32 biomarkers comprise ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J1, and at least one housekeeping gene; (b) normalizing the expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of the at least one housekeeping gene, thereby obtaining a normalized expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1; (c) inputting each normalized expression level into an algorithm to generate a score; (d) comparing the score with a third predetermined cutoff value; and (e) producing a report, wherein the report identifies that the subject has a high risk of disease relapse when the score is equal to or greater than the third predetermined cutoff value or identifies that the subject has a low risk of disease relapse when the score is below the third predetermined cutoff value, wherein the third predetermined cutoff value is 40 on a scale of 0 to 100.

The third predetermined cutoff value can be derived from a plurality of reference samples obtained from subjects whose plasma cell dyscrasias are being adequately controlled by therapies.

The present disclosure also provides a method for determining a response by a subject having a plasma cell dyscrasia to a therapy, comprising: (a) determining a first expression level of at least 31 biomarkers from a first test sample from the subject at a first time point by contacting the first test sample with a plurality of agents specific to detect the expression of the at least 31 biomarkers, wherein the at least 31 biomarkers comprise ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1; (b) determining a second expression level of the at least 31 biomarkers from a second test sample from the subject at a second time point by contacting the second test sample with a plurality of agents specific to detect the expression of the at least 31 biomarkers, wherein the second time point is after the first time point and after the administration of the therapy to the subject; (c) comparing the first expression level with the second expression level; and (d) producing a report, wherein the report identifies that the subject is responsive to the therapy when the second expression level is significantly decreased as compared to the first expression level.

In some embodiments, the methods can predict treatment responsiveness to, or determine whether a patient has become clinically stable following, or is responsive or non-responsive to, a plasma cell dyscrasia treatment, such as a drug therapy (for example, an immunotherapy or targeted therapy). In some cases, the methods can do so with a specificity, sensitivity, and/or accuracy of at least 75%, 80%, 85%, 90%, 91%, 92%, 93%, 94%, 95%, 96%, 97%, 98%, or 99%.

In some embodiments, the first and second test samples can be of the same type. In some embodiments, the first and second test samples can be of different types.

In some embodiments, the therapy can be a drug therapy. The drug therapy can be an immunotherapy, a targeted therapy, a chemotherapy, or a combination thereof. In some embodiments, the therapy can be a radiation therapy.

In some embodiments, the first time point is prior to the administration of the therapy to the subject. In some embodiments, the first time point is after the administration of the therapy to the subject. The second time point can be a few days, a few weeks, or a few months after the first time point. For example, the second time point can be at least 1 day, at least 7 days, at least 14 days, at least 30 days, at least 60 days, or at least 90 days after the first time point.

In some embodiments, the second expression level is significantly decreased as compared to the first expression level when the second expression level is at least 10% less than the first expression level. In some embodiments, the second expression level is significantly decreased as compared to the first expression level when the second expression level is at least 20% less than the first expression level. In some embodiments, the second expression level is significantly decreased as compared to the first expression level when the second expression level is at least 25% less than the first expression level. In some embodiments, the second expression level is significantly decreased as compared to the first expression level when the second expression level is at least 30% less than the first expression level. In some embodiments, the second expression level is significantly decreased as compared to the first expression level when the second expression level is at least 40% less than the first expression level. In some embodiments, the second expression level is significantly decreased as compared to the first expression level when the second expression level is at least 50% less than the first expression level. In some embodiments, the second expression level is significantly decreased as compared to the first expression level when the second expression level is at least 60% less than the first expression level. In some embodiments, the second expression level is significantly decreased as compared to the first expression level when the second expression level is at least 70% less than the first expression level. In some embodiments, the second expression level is significantly decreased as compared to the first expression level when the second expression level is at least 80% less than the first expression level. In some embodiments, the second expression level is significantly decreased as compared to the first expression level when the second expression level is at least 90% less than the first expression level.

In some embodiments, the method further comprises determining a third expression level of the at least 32 biomarkers from a third test sample from the subject at a third time point by contacting the third test sample with a plurality of agents specific to detect the expression of the at least 32 biomarkers, wherein the third time point is after the second time point. The method can further comprise creating a plot showing the trend of the expression level change.

The present disclosure also provides an assay comprising: (a) determining the expression level of biomarkers consisting essentially of the following 32 biomarkers from a test sample from a patient diagnosed of a plasma cell dyscrasia or a subject suspected of having a plasma cell dyscrasia: ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J1, and at least one housekeeping gene; (b) normalizing the expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of the at least one housekeeping gene, thereby obtaining a normalized expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1; (c) inputting each normalized expression level into an algorithm to generate a score; and (d) comparing the score with a first predetermined cutoff value.

The present disclosure also provides an assay comprising: (a) determining the expression level of biomarkers consisting of the following 32 biomarkers from a test sample from patient diagnosed of a plasma cell dyscrasia or a subject suspected of having a plasma cell dyscrasia: ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J1, and at least one housekeeping gene; (b) normalizing the expression level of each of ASXL1, BHLHE40, BTG2, COPA, FBXW7, GNA13, IL8, JMJD1C, LARS2, MALAT1, MBNL1, MCL1, NEKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of the at least one housekeeping gene, thereby obtaining a normalized expression level of each of ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1; (c) inputting each normalized expression level into an algorithm to generate a score; and (d) comparing the score with a first predetermined cutoff value.

In some embodiments, the at least one housekeeping gene is selected from the group consisting of ALG9, SEPN, YWHAQ, VPS37A, PRRC2B, DOPEY2, NDUFB11, ND4, MRPL19, PSMC4, SF3A1, PUM1, ACTB, GAPD, GUSB, RPLP0, TFRC, MORF4L1, 18S, PPIA, PGK1, RPL13A, B2M, YWHAZ, SDHA, HPRT1, TOX4, and TPT1. In some embodiments, the housekeeping gene is TPT1.

In some embodiments, two or more housekeeping genes can be used in normalizing the expression levels. For example, when two housekeeping genes are used, the method can comprise: (1) normalizing the expression level of each of ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of a first housekeeping gene; (2) normalizing the expression level of each of ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, and UBE2J1 to the expression level of a second housekeeping gene; and (3) averaging the first normalized expression level and the second normalized expression level to obtain an averaged normalized expression level.

The sequence information of the plasma cell dyscrasia biomarkers and housekeepers is shown in Table 1.

TABLE 1
Plasma cell dyscrasia Biomarker/Housekeeper Sequence Information

| Gene | RefSeq | | SEQ ID |
|---|---|---|---|
| Name | Accession | Sequence | NO: |
| ASXL1 | NM_001164603.1 | CACACCCACG GCAGACACGC ACGCACCCGG GCGCCGAAGG | 1 |
| | | GAAAGCCGCG TCTCGCCCTC CCGCCCCGCC GTCGGTCCTG | |
| | | TCTCAGTCCC TCAGCAGAGC GGGAAAGCGG AGGCCGGAGC | |
| | | CGTGACCTCT GACCCCGTGG TTATGCGGAG CCGCCGCATT | |
| | | CCTTAGCGAT CGCGGGGCAG CCGCCGCTGC CGCCGTGGGC | |
| | | GACTGACGCA GCGCGGGCGC GTGGAGCCGC CGCCGCCCCT | |
| | | CCCCCACCGC CGCTCTCGCG CCAGCCGGTC CCCGCGTGCC | |
| | | CGCCCCTTCT CCCCGGCCGC ACCCGAGACC TCGCGCGCCG | |
| | | CCGCTGCCAC GCGCCCCCCC CACCGCCGCC GCCGCCCCAG | |
| | | CCCCGCGCCA CCGCCCCAGC CCGCCCAGCC CGGAGGTCCC | |
| | | GCGTGGAGCT GCCGCCGCCG CCGGGGAGAA GGATGAAGGA | |
| | | CAAACAGAAG AAGAAGAAGG AGCGCACGTG GGCCGAGGCC | |
| | | GCGCGCCTGG TATTAGAAAA CTACTCGGAT GCTCCAATGA | |
| | | CACCAAAACA GATTCTGCAG GTCATAGAGG CAGAAGGACT | |
| | | AAAGGAAATG AGAAGTGGGA CTTCCCCTCT CGCATGCCTC | |
| | | AATGCTATGC TACATTCCAA TTCAAGAGGA GGAGAGGGGT | |
| | | TGTTTTATAA ACTGCCTGGC CGAATCAGCC TTTTCACGCT | |
| | | CAAGGTGTGA GCCACTGCAC CAGGCCCCTT CATCTTAATT | |
| | | TTAATATATC TTTGAATAAA CACCATTGTA TGAACCTGCT | |
| | | GTAAGCTTGG GAGTGGTCTG TTAGTCTACA GCTTGTGTCT | |
| | | GAGATGTGCT AATTGAATAT TTGCTCAGTA CCTCATCTTA | |
| | | ACTGCCTTTG GCTTTATGTT GCTTATCCTT CATAGTATCT | |
| | | TGTTCATTGG CCTTTTACAT CCATAGGCAT CACTTCTCTG | |
| | | ATATTCGTTG TGCTCTTTTA ATGGATTAAT GGTTTGCTTG | |
| | | GTTGGTTCCT CTAGTTAGAC TGTAAACTCC TTGAGAGCAG | |
| | | AGTCTGTATT TTATTAATTA CCCACAGTAC TAGGTACATA | |
| | | GTTGCCTTCA ATAAATATAT ATTTAATGAA AAAAAAAAAA | |
| | | AAAA | |
| BHLHE40 | NM_003670.2 | CGCCTCCCCG CCCGCCCCAC TTCTCATTCA CTTGGCTCGC | 2 |
| | | ACGGCGCAGA CAGACCGCGC AGGGAGCACA CACCGCCAGT | |
| | | CTGTGCGCTG AGTCGGAGCC AGAGGCCGCG GGGACACCGG | |
| | | GCCATGCACG CCCCCAACTG AAGCTGCATC TCAAAGCCGA | |
| | | AGATTCCAGC AGCCCAGGGG ATTTCAAAGA GCTCAGACTC | |
| | | AGAGGAACAT CTGCGGAGAG ACCCCCGAAG CCCTCTCCAG | |
| | | GGCAGTCCTC ATCCAGACGC TCCGCTAGTG CAGACAGGAG | |
| | | CGCGCAGTGG CCCCGGCTCG CCGCGCCATG GAGCGGATCC | |
| | | CCAGCGCGCA ACCACCCCCC GCCTGCCTGC CCAAAGCACC | |
| | | GGGACTGGAG CACGGAGACC TACCAGGGAT GTACCCTGCC | |
| | | CACATGTACC AAGTGTACAA GTCAAGACGG GGAATAAAGC | |
| | | GGAGCGAGGA CAGCAAGGAG ACCTACAAAT TGCCGCACCG | |
| | | GCTCATCGAG AAAAAGAGAC GTGACCGGAT TAACGAGTGC | |
| | | ATCGCCCAGC TGAAGGATCT CCTACCCGAA CATCTCAAAC | |
| | | TTACAACTTT GGGTCACTTG GAAAAAGCAG TGGTTCTTGA | |
| | | ACTTACCTTG AAGCATGTGA AAGCACTAAC AAACCTAATT | |
| | | GATCAGCAGC AGCAGAAAAT CATTGCCCTG CAGAGTGGTT | |
| | | TACAAGCTGG TGAGCTGTCA GGGAGAAATG TCGAAACAGG | |
| | | TCAAGAGATG TTCTGCTCAG GTTTCCAGAC ATGTGCCCGG | |
| | | GAGGTGCTTC AGTATCTGGC CAAGCACGAG AACACTCGGG | |
| | | ACCTGAAGTC TTCGCAGCTT GTCACCCACC TCCACCGGGT | |
| | | GGTCTCGGAG CTGCTGCAGG GTGGTACCTC CAGGAAGCCA | |
| | | TCAGACCCAG CTCCCAAAGT GATGGACTTC AAGGAAAAAC | |
| | | CCAGCTCTCC GGCCAAAGGT TCGGAAGGTC CTGGGAAAAA | |
| | | CTGCGTGCCA GTCATCCAGC GGACTTTCGC TCACTCGAGT | |
| | | GGGGAGCAGA GCGGCAGCGA CACGGACACA GACAGTGGCT | |
| | | ATGGAGGAGA ATCGGAGAAG GGCGACTTGC GCAGTGAGCA | |
| | | GCCGTGCTTC AAAAGTGACC ACGGACGCAG GTTCACGATG | |
| | | GGAGAAAGGA TCGGCGCAAT TAAGCAAGAG TCCGAAGAAC | |
| | | CCCCCACAAA AAAGAACCGG ATGCAGCTTT CGGATGATGA | |
| | | AGGCCATTTC ACTAGCAGTG ACCTGATCAG CTCCCCGTTC | |
| | | CTGGGCCCAC ACCCACACCA GCCTCCTTTC TGCCTGCCCT | |
| | | TCTACCTGAT CCCACCTTCA GCGACTGCCT ACCTGCCCAT | |
| | | GCTGGAGAAG TGCTGGTATC CCACCTCAGT GCCAGTGCTA | |
| | | TACCCAGGCC TCAACGCCTC TGCCGCAGCC CTCTCTAGCT | |
| | | TCATGAACCC AGACAAGATC TCGGCTCCCT TGCTCATGCC | |
| | | CCAGAGACTC CCTTCTCCCT TGCCAGCTCA TCCGTCCGTC | |
| | | GACTCTTCTG TCTTGCTCCA AGCTCTGAAG CCAATCCCCC | |
| | | CTTTAAACTT AGAAACCAAA GACTAAACTC TCTAGGGGAT | |
| | | CCTGCTGCTT TGCTTTCCTT CCTCGCTACT TCCTAAAAAG | |
| | | CAACAAAAAA GTTTTTGTGA ATGCTGCAAG ATTGTTGCAT | |
| | | TGTGTATACT GAGATAATCT GAGGCATGGA GAGCAGATTC | |
| | | AGGGTGTGTG TGTGTGTGTG TGTGTGTGTA TGTGCGTGTG | |
| | | CGTGCACATG TGTGCCTGCG TGTTGGTATA GGACTTTAAA | |
| | | GCTCCTTTTG GCATAGGGAA GTCACGAAGG ATTGCTTGAC | |
| | | ATCAGGAGAC TTGGGGGGGA TTGTAGCAGA CGTCTGGGCT | |
| | | TTTCCCCACC CAGAGAATAG CCCCCTTCGA TACACATCAG | |
| | | CTGGATTTTC AAAAGCTTCA AAGTCTTGGT CTGTGAGTCA | |
| | | CTCTTCAGTT TGGGAGCTGG GTCTGTGGCT TTGATCAGAA | |
| | | GGTACTTTCA AAAGAGGGCT TTCCAGGGCT CAGCTCCCAA | |
| | | CCAGCTGTTA GGACCCCACC CTTTTGCCTT TATTGTCGAC | |
| | | GTGACTCACC AGACGTCGGG GAGAGAGAGC AGTCAGACCG | |
| | | AGCTTTCTGC TAACATGGGG AGGTAGCAGG CACTGGCATA | |
| | | GCACGGTAGT GGTTTGGGGA GGTTTCCGCA GGTCTGCTCC | |
| | | CCACCCCTGC CTCGGAAGAA TAAAGAGAAT GTAGTTCCCT | |
| | | ACTCAGGCTT TCGTAGTGAT TAGCTTACTA AGGAACTGAA | |
| | | AATGGGCCCC TTGTACAAGC TGAGCTGCCC CGGAGGGAGG | |
| | | GAGGAGTTCC CTGGGCTTCT GGCACCTGTT TCTAGGCCTA | |
| | | ACCATTAGTA CTTACTGTGC AGGGAACCAA ACCAAGGTCT | |
| | | GAGAAATGCG GACACCCCGA GCGAGCACCC CAAAGTGCAC | |
| | | AAAGCTGAGT AAAAAGCTGC CCCCTTCAAA CAGAACTAGA | |
| | | CTCAGTTTTC AATTCCATCC TAAAACTCCT TTTAACCAAG | |
| | | CTTAGCTTCT CAAAGGCCTA ACCAAGCCTT GGCACCGCCA | |
| | | GATCCTTTCT GTAGGCTAAT TCCTCTTGCC CAACGGCATA | |
| | | TGGAGTGTCC TTATTGCTAA AAAGGATTCC GTCTCCTTCA | |
| | | AAGAAGTTTT ATTTTTGGTC CAGAGTACTT GTTTTCCCGA | |
| | | TGTGTCCAGC CAGCTCCGCA GCAGCTTTTC AAAATGCACT | |
| | | ATGCCTGATT GCTGATCGTG TTTTAACTTT TTCTTTTCCT | |
| | | GTTTTTATTT TGGTATTAAG TCGTTGCCTT TATTTGTAAA | |
| | | GCTGTTATAA ATATATATTA TATAAATATA TTAAAAAGGA | |
| | | AAATGTTTCA GATGTTTATT TGTATAATTA CTTGATTCAC | |
| | | ACAGTGAGAA AAAATGAATG TATTCCTGTT TTTGAAGAGA | |
| | | AGAATAATTT TTTTTTTCTC TAGGGAGAGG TACAGTGTTT | |
| | | ATATTTTGGA GCCTTCCTGA AGGTGTAAAA TTGTAAATAT | |
| | | TTTTATCTAT GAGTAAATGT TAAGTAGTTG TTTTAAAATA | |
| | | CTTAATAAAA TAATTCTTTT CCTGTGGAAG AGAAAAAAAA | |
| | | AAAAAAAAAA AAAAAAAAAA A | |
| BTG2 | NM_006763.2 | CAGGGTAACG CTGTCTTGTG GACCCGCACT TCCCACCCGA | 3 |
| | | GACCTCTCAC TGAGCCCGAG CCGCGCGCGA CATGAGCCAC | |
| | | GGGAAGGGAA CCGACATGCT CCCGGAGATC GCCGCCGCCG | |
| | | TGGGCTTCCT CTCCAGCCTC CTGAGGACCC GGGGCTGCGT | |
| | | GAGCGAGCAG AGGCTTAAGG TCTTCAGCGG GGCGCTCCAG | |
| | | GAGGCACTCA CAGAGCACTA CAAACACCAC TGGTTTCCCG | |
| | | AAAAGCCGTC CAAGGGCTCC GGCTACCGCT GCATTCGCAT | |
| | | CAACCACAAG ATGGACCCCA TCATCAGCAG GGTGGCCAGC | |
| | | CAGATCGGAC TCAGCCAGCC CCAGCTGCAC CAGCTGCTGC | |
| | | CCAGCGAGCT GACCCTGTGG GTGGACCCCT ATGAGGTGTC | |
| | | CTACCGCATT GGGGAGGACG GCTCCATCTG CGTCTTGTAC | |
| | | GAGGAGGCCC CACTGGCCGC CTCCTGTGGG CTCCTCACCT | |
| | | GCAAGAACCA AGTGCTGCTG GGCCGGAGCA GCCCCTCCAA | |
| | | GAACTACGTG ATGGCAGTCT CCAGCTAGGC CCTTCCGCCC | |
| | | CCGCCCTGGG CGCCGCCGTG CTCATGCTGC CGTGACAACA | |
| | | GGCCACCACA TACCTCAACC TGGGGAACTG TATTTTTAAA | |
| | | TGAAGAGCTA TTTATATATA TTATTTTTTT TTAAGAAAGG | |
| | | AGGAAAAGAA ACCAAAAGTT TTTTTTAAGA AAAAAAATCC | |
| | | TTCAAGGGAG CTGCTTGGAA GTGGCCTCCC CAGGTGCCTT | |
| | | TGGAGAGAAC TGTTGCGTGC TTGAGTCTGT GAGCCAGTGT | |
| | | CTGCCTATAG GAGGGGGAGC TGTTAGGGGG TAGACCTAGC | |
| | | CAAGGAGAAG TGGGAGACGT TTGGCTAGCA CCCCAGGAAG | |
| | | ATGTGAGAGG GAGCAAGCAA GGTTAGCAAC TGTGAACAGA | |
| | | GAGGTCGGGA TTTGCCCTGG GGGAGGAAGA GAGGCCAAGT | |
| | | TCAGAGCTCT CTGTCTCCCC CAGCCAGACA CCTGCATCCC | |
| | | TGGCTCCTCT ATTACTCAGG GGCATTCATG CCTGGACTTA | |
| | | AACAATACTA TGTTATCTTT TCTTTTATTT TTCTAATGAG | |
| | | GTCCTGGGCA GAGAGTGAAA AGGCCTCTCC TGATTCCTAC | |
| | | TGTCCTAAGC TGCTTTTCTT GAAATCATGA CTTGTTTCTA | |
| | | ATTCTACCCT CAGGGGCCTG TAGATGTTGC TTTCCAGCCA | |
| | | GGAATCTAAA GCTTTGGGTT TTCTGAGGGG GGGGAGGAGG | |
| | | GAACTGGAGG TTATTGGGGT TAGGATGGAA GGGAACTCTG | |
| | | CACAAAACCT TTGCTTTGCT AGTGCTGCTT TGTGTGTATG | |
| | | TGTGGCAAAT AATTTGGGGG TGATTTGCAA TGAAATTTTG | |
| | | GGACCCAAAG AGTATCCACT GGGGATGTTT TTTGGCCAAA | |
| | | ACTCTTCCTT TTGGAACCAC ATGAAAGTCT TGATGCTGCT | |
| | | GCCATGATCC CTTTGAGAGG TGGCTCAAAA GCTACAGGGA | |
| | | ACTCCAGGTC CTTTATTACT GCCTTCTTTT CAAAAGCACA | |
| | | ACTCTCCTCT AACCCTCCCC TCCCCCTTCC CTTCTGGTCG | |
| | | GGTCATAGAG CTACCGTATT TTCTAGGACA AGAGTTCTCA | |
| | | GTCACTGTGC AATATGCCCC CTGGGTCCCA GGAGGGTCTG | |
| | | GAGGAAAACT GGCTATCAGA ACCTCCTGAT GCCCTGGTGG | |
| | | GCTTAGGGAA CCATCTCTCC TGCTCTCCTT GGGATGATGG | |
| | | CTGGCTAGTC AGCCTTGCAT GTATTCCTTG GCTGAATGGG | |
| | | AGAGTGCCCC ATGTTCTGCA AGACTACTTG GTATTCTTGT | |
| | | AGGGCCGACA CTAAATAAAA GCCAAACCTT GGGCACTGTT | |
| | | TTTTCTCCCT GGTGCTCAGA GCACCTGTGG GAAAGGTTGC | |
| | | TGTCTGTCTC AGTACAATCC AAATTTGTCG TAGACTTGTG | |
| | | CAATATATAC TGTTGTGGGT TGGAGAAAAG TGGAAAGCTA | |
| | | CACTGGGAAG AAACTCCCTT CCTTCAATTT CTCAGTGACA | |
| | | TTGATGAGGG GTCCTCAAAA GACCTCGAGT TTCCCAAACC | |
| | | GAATCACCTT AAGAAGGACA GGGCTAGGGC ATTTGGCCAG | |
| | | GATGGCCACC CTCCTGCTGT TGCCCCTTAG TGAGGAATCT | |
| | | TCACCCCACT TCCTCTACCC CCAGGTTCTC CTCCCCACAG | |
| | | CCAGTCCCCT TTCCTGGATT TCTAAACTGC TCAATTTTGA | |
| | | CTCAAAGGTG CTATTTACCA AACACTCTCC CTACCCATTC | |
| | | CTGCCAGCTC TGCCTCCTTT TCAACTCTCC ACATTTTGTA | |
| | | TTGCCTTCCC AGACCTGCTT CCAGTCTTTA TTGCTTTAAA | |
| | | GTTCACTTTG GGCCCACAGA CCCAAGAGCT AATTTTCTGG | |
| | | TTTGTGGGTT GAAACAAAGC TGTGAATCAC TGCAGGCTGT | |
| | | GTTCTTGCAT CTTGTCTGCA AACAGGTCCC TGCCTTTTTA | |
| | | GAAGCAGCCT CATGGTCTCA TGCTTAATCT TGTCTCTCTT | |
| | | CTCTTCTTTA TGATGTTCAC TTTAAAAACA ACAAAACCCC | |
| | | TGAGCTGGAC TGTTGAGCAG GCCTGTCTCT CCTATTAAGT | |
| | | AAAAATAAAT AGTAGTAGTA TGTTTGTAAG CTATTCTGAC | |
| | | AGAAAAGACA AAGGTTACTA ATTGTATGAT AGTGTTTTTA | |
| | | TATGGAAGAA TGTACAGCTT ATGGACAAAT GTACACCTTT | |
| | | TTGTTACTTT AATAAAAATG TAGTAGGATA AAAAAAAA | |
| COPA | NM_001098398.1 | AGAAGGGAGA CAGTGGCGGT CGCGGCGGGG CCGATCCGAG | 4 |
| | | AGTTCCCCTT AGAGAACGGA GCTCACGGGC GGGGAGGCCT | |
| | | CACCTGCTAG TAGGACGCAG AAAGACAGAA GGCGAAGGAG | |
| | | ACCCCGACTT CCCGGGTCAG CCCCAGAGCC ACCCCCTGCC | |
| | | GTAGCCATCT TGCCTCTCTG CTGAGCGGAA GCCCCCGTTC | |
| | | GGCTCCTGTC TGTTAGCGGC CTCTCTAGGC TACCACTGAC | |
| | | ACCGTCTCTG TGGCCCGGAG CCTAAGAGAC CGGAAGTTCG | |
| | | TGTTTCCAGG CGCTTCCGGA AACCGCGGGA GAGGGTCGCT | |
| | | GACGTGGAGG CGTCCGAAGG GCAGCAGGGT GTGTCGGGGC | |
| | | TCGGATTAAG ACATCGGAGT CGGAGACCTG AGAGATGTTA | |
| | | ACCAAATTCG AGACCAAGAG CGCGCGGGTC AAAGGGCTCA | |
| | | GCTTTCACCC CAAAAGACCT TGGATCCTGA CTAGTTTACA | |
| | | TAATGGGGTC ATCCAGTTAT GGGACTATCG GATGTGCACT | |
| | | CTCATTGACA AGTTTGATGA ACATGATGGT CCAGTGCGAG | |
| | | GCATTGACTT CCATAAGCAG CAGCCACTGT TCGTCTCTGG | |
| | | AGGAGATGAC TATAAGATTA AGGTTTGGAA TTACAAGCTT | |
| | | CGGCGCTGTC TTTTCACATT GCTTGGGCAC TTAGATTATA | |
| | | TTCGCACCAC GTTTTTTCAT CATGAATATC CCTGGATTCT | |
| | | GAGTGCCTCC GATGATCAGA CCATCCGAGT GTGGAACTGG | |
| | | CAATCTAGAA CCTGTGTTTG TGTGTTAACA GGGCACAACC | |
| | | ATTATGTGAT GTGTGCTCAG TTCCACCCCA CAGAAGACTT | |
| | | GGTAGTATCA GCCAGCCTGG ACCAGACTGT GCGCGTTTGG | |
| | | GATATTTCTG GTCTGAGGAA AAAAAACCTG TCCCCTGGTG | |
| | | CGGTGGAATC GGATGTGAGA GGAATAACTG GGGTTGATCT | |
| | | ATTTGGAACT ACAGATGCAG TGGTGAAGCA TGTACTAGAG | |
| | | GGTCACGATC GTGGAGTAAA CTGGGCTGCC TTCCACCCCA | |
| | | CTATGCCCCT TATTGTATCT GGGGCAGATG ATCGTCAAGT | |
| | | GAAGATCTGG CGCATGAATG AATCAAAGGC ATGGGAGGTT | |
| | | GATACCTGCC GGGGCCATTA CAACAATGTA TCTTGTGCCG | |
| | | TCTTCCACCC TCGCCAAGAG TTGATCCTCA GCAATTCTGA | |
| | | GGACAAGAGT ATTCGAGTCT GGGATATGTC TAAGCGGACT | |
| | | GGGGTTCAGA CTTTCCGCAG AGACCATGAT CGTTTCTGGG | |
| | | TCCTAGCTGC TCACCCTAAC CTTAACCTCT TTGCAGCAGG | |
| | | CCATGATGGT GGTATGATTG TGTTTAAGCT GGAACGGGAA | |
| | | CGGCCAGCCT ATGCTGTTCA TGGCAATATG CTACACTATG | |
| | | TCAAGGACCG ATTCTTACGA CAGCTGGATT TCAACAGCTC | |
| | | CAAAGATGTA GCTGTGATGC AGTTGCGGAG TGGTTCCAAG | |
| | | TTTCCAGTAT TCAATATGTC ATACAATCCA GCAGAAAATG | |
| | | CAGTCCTGCT TTGTACAAGA GCTAGCAATC TAGAGAATAG | |
| | | TACCTATGAC CTGTACACCA TCCCTAAAGA TGCTGACTCC | |
| | | CAGAATCCTG ATGCGCCTGA AGGGAAACGA TCCTCAGGCC | |
| | | TGACAGCCGT TTGGGTCGCT CGAAATCGGT TTGCTGTCCT | |
| | | AGATCGGATG CATTCGCTTC TGATCAAGAA TCTGAAGAAT | |
| | | GAGATCACCA AAAAGGTACA GGTGCCCAAC TGTGATGAGA | |
| | | TCTTCTATGC TGGCACAGGC AATCTCCTGC TTCGAGATGC | |
| | | GGACTCTATC ACACTCTTTG ACGTACAGCA GAAGCGGACT | |
| | | CTGGCATCTG TGAAGATTTC TAAAGTGAAA TACGTTATCT | |
| | | GGTCAGCAGA CATGTCACAT GTAGCACTAC TAGCCAAACA | |
| | | CGAACACTCA TGCCCTTTGC CTCTTACAGC CATTGTGATC | |
| | | TGTAACCGCA AACTGGATGC TTTATGTAAC ATTCATGAGA | |
| | | ACATTCGTGT CAAGAGTGGG GCCTGGGATG AGAGTGGGGT | |
| | | ATTTATCTAT ACCACAAGCA ACCACATCAA ATATGCTGTC | |
| | | ACCACTGGGG ACCACGGGAT CATTCGAACT CTGGATTTAC | |
| | | CCATCTATGT CACACGGGTG AAGGGCAACA ATGTATACTG | |
| | | CCTAGACAGG GAGTGTCGTC CCCGGGTACT CACCATTGAT | |
| | | CCCACTGAGT TCAAATTCAA GCTGGCCCTG ATCAACAGAA | |
| | | AATATGATGA GGTACTGCAC ATGGTGAGGA ATGCCAAACT | |
| | | AGTTGGCCAG TCTATTATTG CTTATCTCCA GAAGAAGGGC | |
| | | TATCCTGAAG TGGCACTGCA TTTTGTCAAG GATGAGAAAA | |
| | | CTCGCTTTAG TCTGGCACTG GAGTGTGGAA ACATTGAGAT | |
| | | TGCTCTGGAA GCAGCCAAAG CACTGGATGA CAAGAACTGC | |
| | | TGGGAAAAGC TGGGAGAAGT GGCCCTGCTG CAGGGGAACC | |
| | | ACCAGATTGT GGAAATGTGC TATCAGCGTA CCAAAAACTT | |
| | | TGACAAACTT TCCTTCCTGT ATCTTATCAC TGGCAACTTA | |
| | | GAAAAACTTC GCAAGATGAT GAAGATTGCT GAGATCAGAA | |
| | | AGGACATGAG TGGCCACTAT CAGAATGCCC TATACCTGGG | |
| | | TGATGTGTCA GAGCGTGTGC GGATCCTGAA GAACTGTGGA | |
| | | CAGAAGTCCC TGGCCTATCT CACAGCTGCT ACCCATGGCT | |
| | | TAGATGAAGA AGCTGAGAGC CTAAAGGAGA CATTTGACCC | |
| | | AGAGAAGGAG ACAATCCCAG ACATTGACCC TAATGCCAAG | |
| | | CTGCTCCAGC CACCTGCACC TATCATGCCA TTGGATACCA | |
| | | ATTGGCCTTT ATTGACTGTA TCCAAAGGAT TTTTTGAAGG | |
| | | CACCATTGCC AGCAAAGGGA AGGGAGGAGC ACTGGCTGCT | |
| | | GACATTGACA TTGACACTGT TGGTACAGAG GGCTGGGGAG | |
| | | AGGATGCAGA GCTGCAGTTG GATGAAGATG GGTTTGTGGA | |
| | | GGCTACAGAA GGTTTGGGGG ATGATGCTCT TGGCAAGGGA | |
| | | CAGGAAGAAG GAGGTGGCTG GGATGTAGAA GAAGATCTGG | |
| | | AGCTCCCTCC TGAGCTGGAT ATATCCCCTG GGGCAGCTGG | |
| | | TGGGGCTGAA GATGGTTTCT TTGTGCCCCC AACCAAGGGA | |
| | | ACAAGTCCAA CTCAGATCTG GTGTAATAAC TCTCAGCTTC | |
| | | CAGTTGATCA CATCCTGGCA GGCTCTTTCG AAACAGCCAT | |
| | | GCGGCTCCTT CATGACCAAG TAGGGGTAAT CCAGTTTGGC | |
| | | CCCTACAAGC AACTGTTCCT ACAGACATAC GCCCGAGGCC | |
| | | GCACAACCTA TCAGGCTCTG CCCTGCCTAC CCTCCATGTA | |
| | | TGGCTATCCT AATCGCAACT GGAAGGATGC AGGGCTGAAG | |
| | | AATGGTGTAC CAGCTGTGGG CCTGAAGCTT AATGACCTCA | |
| | | TCCAACGGTT GCAGCTGTGC TACCAGCTCA CCACAGTTGG | |
| | | CAAATTTGAG GAGGCTGTGG AAAAATTCCG TTCCATCCTT | |
| | | CTCAGTGTGC CACTTCTTGT TGTGGACAAT AAACAAGAGA | |
| | | TTGCAGAGGC CCAGCAGCTC ATCACCATTT GCCGTGAGTA | |
| | | CATTGTGGGT TTGTCCGTGG AGACAGAAAG GAAGAAGCTG | |
| | | CCCAAAGAGA CTCTAGAACA GCAGAAGCGC ATCTGTGAGA | |
| | | TGGCAGCCTA TTTCACCCAC TCAAACCTGC AGCCTGTGCA | |
| | | CATGATCCTG GTGCTGCGTA CAGCCCTCAA TCTGTTCTTC | |
| | | AAGCTCAAGA ACTTCAAGAC AGCTGCCACC TTTGCTCGGC | |
| | | GCCTACTAGA ACTCGGGCCC AAGCCTGAGG TGGCCCAACA | |
| | | GACCCGAAAA ATCCTGTCTG CCTGTGAGAA GAATCCCACA | |
| | | GATGCCTACC AGCTCAATTA TGACATGCAC AACCCCTTTG | |
| | | ACATTTGTGC TGCATCATAT CGGCCCATCT ACCGTGGAAA | |
| | | GCCAGTAGAA AAGTGTCCAC TCAGTGGGGC CTGCTATTCC | |
| | | CCTGAGTTCA AAGGTCAAAT CTGCAGGGTC ACCACAGTGA | |
| | | CAGAGATTGG CAAAGATGTG ATTGGTTTAA GGATCAGTCC | |
| | | TCTGCAGTTT CGCTAAGGCC CCCTTTGTGT GCATGGGTCA | |
| | | GTCACCATAT GTTCCCCCCA GAGAATGTGT CTATATCCTC | |
| | | CTTCTAACAG CACCTTCCCC CTGCAGCTAC TCTTCAGATC | |
| | | TGGCTCTCTG TACCCTAAAA CCTAGTATCT TTTTCTCTTC | |
| | | TATGGAAAAT CCGAAGGTCT AAACTTGACT TTTTTGAGGT | |
| | | CTTCTCAACT TGACTACAGT TGTGCTCATA ATTGTCCTTG | |
| | | CCTTTCCAGC TTAATTATTT TAAGGAACAA ATGAAAACTC | |
| | | TGGGCTGGGT GGAGTGGCTC ATACCTGTAA TCCCAGCACT | |
| | | TTGGGAGGCT ACGGTGGGCA GATCATCTGA GGCCAGGAGT | |
| | | TCGAGACCTG CCTGGCCAAC ATGGCAACAC CCCGTCTCTA | |
| | | ATAAAAATAT AAAAATTAGC CTGGCATGGT AGCATGCGCC | |
| | | TATAGTCCCA GCTGCTCAGG AGGCTGAGGC ATGAGAATCG | |
| | | CTTGAACCTA GGAGGTGGAG GTTGCATTCA ACTGAGATCA | |
| | | TACCACTTCA TTCCAGCCTG GGTGACAGAG CAAGACTCTG | |
| | | TCTCAAAAAA AAAAAAAAGG AAAACTCTGT GATGGACATT | |
| | | TGTTTAGTAA ATCCCTTCAG TATTTATCCC TCCTTTCCCC | |
| | | ACAGCAGCTT TCTTTCCTGT CAACTAGAAA GGAGCAGGAT | |
| | | GTAATAAATA CATTTTGGTG TGACTAGGCC ACACCAACTC | |
| | | TTAATCATCT CCCATTTTCC TTAGACATTT AAATTTCAAG | |
| | | GCAGGTACCC TCTGTGTACT CAGAAATTTG AAGAAGTTAT | |
| | | TTGGTTTTCC AAAATGCACA CTGCGGGTTA TTGATTTGTT | |
| | | CTTTACAACT ATTGTTCTCA TATTTCTCAC ACTAAATAAA | |
| | | TCTCTATGAG AGCTTCTTGA CTTGGCCATT TATTTCTTGG | |
| | | ACACTCTCAT GTTCTTGTTC ACCCATGCAG GCACCCCACC | |
| | | AAAGTACATA TCTTCCTTCC AGTAATAATT TTTAATTACA | |
| | | AAATAAACAT CCACTATTGG AAAAAAAAAA AAAAAGCTAG | |
| | | CCGGGCATGG TGGTGGGTGC CTGTAATCCC AGCTACTCTG | |
| | | GAGGCTGAGG CAGAGGATTG CTTGAACCCG GGAGGCGGAG | |
| | | GTTGCAGTAA GCTGAGATCG CGCCACCGCA CTCCAGCCTG | |
| | | GGCGACAGAG TGAGACTCCA TCTCAAAAAA AAAGAAAGAA | |
| | | AAAAAGAAGC ACATGTTTTT CATAGGGTAT ATATGAGGAC | |
| | | CTAAACTGCT GTGAAAATGA TAGAAAGCAA GTAGCTCCCT | |
| | | TATTCTGTTT TTGATTGCAG CCTTTTATCT TTTGCTAATT | |
| | | ATAGCAATAT TTATTGAGCA CCTGCCATGT GACTGTCACT | |
| | | GTTCTAGATA TTTTACATGT AATATACAGA TAAAAGAATA | |
| | | GTACTTTATA TATATTACAA TGATACAATG ATTACATTAA | |
| | | CAATACAATA TTTTGCTTGT CATATGCTAA GAATAATTGG | |
| | | GTAGAGTGAC ATTACTGTGC CTTCGATTAA AATAAGTACT | |
| | | TTTTTGCGTG TTAAATTCAT GTTTTCAATA AATAATAAAT | |
| | | GCATATAGTT GAAAAATCAG TAAATA | |
| FBXW7 | NM_001013415.1 | AGACAGGTCA GGACATTTGG TAGGGGAAGG TTGAAAGACA | 5 |
| | | AAAGCAGCAG GCCTTGGGTT CTCAGCCTTT TAAAAACTAT | |
| | | TATTAAATAT ATATTTTTAA AATTTAGTGG TTAGAGCTTT | |
| | | TAGTAATGTG CCTGTATTAC ATGTAGAGAG TATTCGTCAA | |
| | | CCAAGAGGAG TTTTAAAATG TCAAAACCGG GAAAACCTAC | |
| | | TCTAAACCAT GGCTTGGTTC CTGTTGATCT TAAAAGTGCA | |
| | | AAAGAGCCTC TACCACATCA AACTGTGATG AAGATATTTA | |
| | | GCATTAGCAT CATTGCCCAA GGCCTCCCTT TTTGTCGAAG | |
| | | ACGGATGAAA AGAAAGTTGG ACCATGGTTC TGAGGTCCGC | |
| | | TCTTTTTCTT TGGGAAAGAA ACCATGCAAA GTCTCAGAAT | |
| | | ATACAAGTAC CACTGGGCTT GTACCATGTT CAGCAACACC | |
| | | AACAACTTTT GGGGACCTCA GAGCAGCCAA TGGCCAAGGG | |
| | | CAACAACGAC GCCGAATTAC ATCTGTCCAG CCACCTACAG | |
| | | GCCTCCAGGA ATGGCTAAAA ATGTTTCAGA GCTGGAGTGG | |
| | | ACCAGAGAAA TTGCTTGCTT TAGATGAACT CATTGATAGT | |
| | | TGTGAACCAA CACAAGTAAA ACATATGATG CAAGTGATAG | |
| | | AACCCCAGTT TCAACGAGAC TTCATTTCAT TGCTCCCTAA | |
| | | AGAGTTGGCA CTCTATGTGC TTTCATTCCT GGAACCCAAA | |
| | | GACCTGCTAC AAGCAGCTCA GACATGTCGC TACTGGAGAA | |
| | | TTTTGGCTGA AGACAACCTT CTCTGGAGAG AGAAATGCAA | |
| | | AGAAGAGGGG ATTGATGAAC CATTGCACAT CAAGAGAAGA | |
| | | AAAGTAATAA AACCAGGTTT CATACACAGT CCATGGAAAA | |
| | | GTGCATACAT CAGACAGCAC AGAATTGATA CTAACTGGAG | |
| | | GCGAGGAGAA CTCAAATCTC CTAAGGTGCT GAAAGGACAT | |
| | | GATGATCATG TGATCACATG CTTACAGTTT TGTGGTAACC | |
| | | GAATAGTTAG TGGTTCTGAT GACAACACTT TAAAAGTTTG | |
| | | GTCAGCAGTC ACAGGCAAAT GTCTGAGAAC ATTAGTGGGA | |
| | | CATACAGGTG GAGTATGGTC ATCACAAATG AGAGACAACA | |
| | | TCATCATTAG TGGATCTACA GATCGGACAC TCAAAGTGTG | |
| | | GAATGCAGAG ACTGGAGAAT GTATACACAC CTTATATGGG | |
| | | CATACTTCCA CTGTGCGTTG TATGCATCTT CATGAAAAAA | |
| | | GAGTTGTTAG CGGTTCTCGA GATGCCACTC TTAGGGTTTG | |
| | | GGATATTGAG ACAGGCCAGT GTTTACATGT TTTGATGGGT | |
| | | CATGTTGCAG CAGTCCGCTG TGTTCAATAT GATGGCAGGA | |
| | | GGGTTGTTAG TGGAGCATAT GATTTTATGG TAAAGGTGTG | |
| | | GGATCCAGAG ACTGAAACCT GTCTACACAC GTTGCAGGGG | |
| | | CATACTAATA GAGTCTATTC ATTACAGTTT GATGGTATCC | |
| | | ATGTGGTGAG TGGATCTCTT GATACATCAA TCCGTGTTTG | |
| | | GGATGTGGAG ACAGGGAATT GCATTCACAC GTTAACAGGG | |
| | | CACCAGTCGT TAACAAGTGG AATGGAACTC AAAGACAATA | |
| | | TTCTTGTCTC TGGGAATGCA GATTCTACAG TTAAAATCTG | |
| | | GGATATCAAA ACAGGACAGT GTTTACAAAC ATTGCAAGGT | |
| | | CCCAACAAGC ATCAGAGTGC TGTGACCTGT TTACAGTTCA | |
| | | ACAAGAACTT TGTAATTACC AGCTCAGATG ATGGAACTGT | |
| | | AAAACTATGG GACTTGAAAA CGGGTGAATT TATTCGAAAC | |
| | | CTAGTCACAT TGGAGAGTGG GGGGAGTGGG GGAGTTGTGT | |
| | | GGCGGATCAG AGCCTCAAAC ACAAAGCTGG TGTGTGCAGT | |
| | | TGGGAGTCGG AATGGGACTG AAGAAACCAA GCTGCTGGTG | |
| | | CTGGACTTTG ATGTGGACAT GAAGTGAAGA GCAGAAAAGA | |
| | | TGAATTTGTC CAATTGTGTA GACGATATAC TCCCTGCCCT | |
| | | TCCCCCTGCA AAAAGAAAAA AAGAAAAGAA AAAGAAAAAA | |
| | | ATCCCTTGTT CTCAGTGGTG CAGGATGTTG GCTTGGGGCA | |
| | | ACAGATTGAA AAGACCTACA GACTAAGAAG GAAAAGAAGA | |
| | | AGAGATGACA AACCATAACT GACAAGAGAG GCGTCTGCTG | |
| | | TCTCATCACA TAAAAGGCTT CACTTTTGAC TGAGGGCAGC | |
| | | TTTGCAAAAT GAGACTTTCT AAATCAAACC AGGTGCAATT | |
| | | ATTTCTTTAT TTTCTTCTCC AGTGGTCATT GGGCAGTGTT | |
| | | AATGCTGAAA CATCATTACA GATTCTGCTA GCCTGTTCTT | |
| | | TTACCACTGA CAGCTAGACA CCTAGAAAGG AACTGCAATA | |
| | | ATATCAAAAC AAGTACTGGT TGACTTTCTA ATTAGAGAGC | |
| | | ATCTGCAACA AAAAGTCATT TTTCTGGAGT GGAAAAGCTT | |
| | | AAAAAAATTA CTGTGAATTG TTTTTGTACA GTTATCATGA | |
| | | AAAGCTTTTT TTTTTTTTTT TTTGCCAACC ATTGCCAATG | |
| | | TCAATCAATC ACAGTATTAG CCTCTGTTAA TCTATTTACT | |
| | | GTTGCTTCCA TATACATTCT TCAATGCATA TGTTGCTCAA | |
| | | AGGTGGCAAG TTGTCCTGGG TTCTGTGAGT CCTGAGATGG | |
| | | ATTTAATTCT TGATGCTGGT GCTAGAAGTA GGTCTTCAAA | |
| | | TATGGGATTG TTGTCCCAAC CCTGTACTGT ACTCCCAGTG | |
| | | GCCAAACTTA TTTATGCTGC TAAATGAAAG AAAGAAAAAA | |
| | | GCAAATTATT TTTTTTTATT TTTTTTCTGC TGTGACGTTT | |
| | | TAGTCCCAGA CTGAATTCCA AATTTGCTCT AGTTTGGTTA | |
| | | TGGAAAAAAG ACTTTTTGCC ACTGAAACTT GAGCCATCTG | |
| | | TGCCTCTAAG AGGCTGAGAA TGGAAGAGTT TCAGATAATA | |
| | | AAGAGTGAAG TTTGCCTGCA AGTAAAGAAT TGAGAGTGTG | |
| | | TGCAAAGCTT ATTTTCTTTT ATCTGGGCAA AAATTAAAAC | |
| | | ACATTCCTTG GAACAGAGCT ATTACTTGCC TGTTCTGTGG | |
| | | AGAAACTTTT CTTTTTGAGG GCTGTGGTGA ATGGATGAAC | |
| | | GTACATCGTA AAACTGACAA AATATTTTAA AAATATATAA | |
| | | AACACAAAAT TAAAATAAAG TTGCTGGTCA GTCTTAGTGT | |
| | | TTTACAGTAT TTGGGAAAAC AACTGTTACA GTTTTATTGC | |
| | | TCTGAGTAAC TGACAAAGCA GAAACTATTC AGTTTTTGTA | |
| | | GTAAAGGCGT CACATGCAAA CAAACAAAAT GAATGAAACA | |
| | | GTCAAATGGT TTGCCTCATT CTCCAAGAGC CACAACTCAA | |
| | | GCTGAACTGT GAAAGTGGTT TAACACTGTA TCCTAGGCGA | |
| | | TCTTTTTTCC TCCTTCTGTT TATTTTTTTG TTTGTTTTAT | |
| | | TTATAGTCTG ATTTAAAACA ATCAGATTCA AGTTGGTTAA | |
| | | TTTTAGTTAT GTAACAACCT GACATGATGG AGGAAAACAA | |
| | | CCTTTAAAGG GATTGTGTCT ATGGTTTGAT TCACTTAGAA | |
| | | ATTTTATTTT CTTATAACTT AAGTGCAATA AAATGTGTTT | |
| | | TTTCATGTTA | |
| GNA13 | NM_006572.5 | CTCCTTCCCT CCCTCCCTCC CCCGCTGTCC TGGCCCGCCC | 6 |
| | | TGCCCGGCCC GCCCTGCGAG TCAGTTCGCT GGTTCCCTCC | |
| | | CTCCCTGGGC GCGCTCGGGC CGCCGCCGCG CTCCCCGCCC | |
| | | TCGAGCCTCG GTGCCGGAGC CGCCCGCCGC CGGAGGAGGA | |
| | | GGTGGAGGGA GCCGGAGGGG CCCGCCGAGG CGGCGGCGGC | |
| | | GGCGGCAAGA TGGCGGACTT CCTGCCGTCG CGGTCCGTGC | |
| | | TGTCCGTGTG CTTCCCCGGC TGCCTGCTGA CGAGTGGCGA | |
| | | GGCCGAGCAG CAACGCAAGT CCAAGGAGAT CGACAAATGC | |
| | | CTGTCTCGGG AAAAGACCTA TGTGAAGCGG CTGGTGAAGA | |
| | | TCCTGCTGCT GGGCGCGGGC GAGAGCGGCA AGTCCACCTT | |
| | | CCTGAAGCAG ATGCGGATCA TCCACGGGCA GGACTTCGAC | |
| | | CAGCGCGCGC GCGAGGAGTT CCGCCCCACC ATCTACAGCA | |
| | | ACGTGATCAA AGGTATGAGG GTGCTGGTTG ATGCTCGAGA | |
| | | GAAGCTTCAT ATTCCCTGGG GAGACAACTC AAACCAACAA | |
| | | CATGGAGATA AGATGATGTC GTTTGATACC CGGGCCCCCA | |
| | | TGGCAGCCCA AGGAATGGTG GAAACAAGGG TTTTCTTACA | |
| | | ATATCTTCCT GCTATAAGAG CATTATGGGC AGACAGCGGC | |
| | | ATACAGAATG CCTATGACCG GCGTCGAGAA TTTCAACTGG | |
| | | GTGAATCTGT AAAATATTTC CTGGATAACT TGGATAAACT | |
| | | TGGAGAACCA GATTATATTC CATCACAACA AGATATTCTG | |
| | | CTTGCCAGAA GACCCACCAA AGGCATCCAT GAATACGACT | |
| | | TTGAAATAAA AAATGTTCCT TTCAAAATGG TTGATGTAGG | |
| | | TGGTCAGAGA TCAGAAAGGA AACGTTGGTT TGAATGTTTC | |
| | | GACAGTGTGA CATCAATACT TTTCCTTGTT TCCTCAAGTG | |
| | | AATTTGACCA GGTGCTTATG GAAGATCGAC TGACCAATCG | |
| | | CCTTACAGAG TCTCTGAACA TTTTTGAAAC AATCGTCAAT | |
| | | AACCGGGTTT TCAGCAATGT CTCCATAATT CTGTTCTTAA | |
| | | ACAAGACAGA CTTGCTTGAG GAGAAGGTGC AAATTGTGAG | |
| | | CATCAAAGAC TATTTCCTAG AATTTGAAGG GGATCCCCAC | |
| | | TGCTTAAGAG ACGTCCAAAA ATTCCTGGTG GAATGTTTCC | |
| | | GGAACAAACG CCGGGACCAG CAACAGAAGC CCTTATACCA | |
| | | CCACTTCACC ACTGCTATCA ACACGGAGAA CATCCGCCTT | |
| | | GTTTTCCGTG ACGTGAAGGA TACTATTCTG CATGACAACC | |
| | | TCAAGCAGCT TATGCTACAG TGATGTACAA AAGACTTGCT | |
| | | GTTTTAATAT CTTTTTGTGT TTTTGATGTT TTCTGTTTGT | |
| | | TTTGTTTTTT AAAATAGCAG TTTACAACCA GAATTAGAAC | |
| | | AATCTTAATT CTACGTTTAA CTTCTTGAAA ATCTTAGTAC | |
| | | TTTTTCTGCG GCCTTTGGTT TGTGGCTGAA AGCTGTTGAG | |
| | | TGACTCATCG CCAAGATTTG CTGTAATGCA GGCTTTGATC | |
| | | TGTTTCACCA TGGCTTCTAT TCAAGTCCAG TTAAAACCTC | |
| | | CCAGCTGACC TCAGACTAGG CATATTTCAG GCTTTAAATT | |
| | | ATTCTACTTT CCAAACTGAA TTCTCCTGCA GTGCCAAGTA | |
| | | TCAAAGGTGT CCTTAAATAC TTGTAGGGAT GAGGTTAGGA | |
| | | ATATTCAGTT CCAAAACAAG ATATCTTCTG TCCGCCTTAC | |
| | | ATATAGCAGT GACACTTGTT GCCTAACTTT ATGGTGACCT | |
| | | CCTATTTTGT AAGGGCTGTT AGAAGTTCTA TCTAAGAAAT | |
| | | GGCATTCTGT AGGTTTATAG AAGGTTTAGC CTTCATATTT | |
| | | TAATTGCTTG TATACACAAC AGCTGTTTTG CTTTTAGATT | |
| | | TCTGTGTTTC TGAAGGTAAT GTTCTTCCTG TTTTCAAGTT | |
| | | TACATAAGGA TCTTTGGTCT GATGCTGATG AAGAGTTCAC | |
| | | AGGTGGTATG GGAGAGCAAA AGGCAAGCTA ATGCTGTTTA | |
| | | CCGTGTTTTG GTCAAACGTA ACGAGTGAAA TAGAATTTGC | |
| | | CTTTCTCATA TTTAATTATC ATGTAGTTTA ATGTACCATA | |
| | | TGTGAAACAT TCTGGCCATA GCAGCAACTA AAAACTGCAA | |
| | | GCAACTTGGT AACAGAACTT TCTAAATAAA CTTAACCTGT | |
| | | TCCAGAATGT CATGTATTTG ACTTTTAAGC CCTATCTCAG | |
| | | TTGGTCAGTA AAGACCAATC CTTACTGTAG GAAATCATTG | |
| | | TTGTATCATC ACAAACATCT ATCTTTTGCT GTCCTGTCCA | |
| | | GTCCCATCAA CTCCACACTG TGCCATTTGT GGCATCGTTT | |
| | | TGTTTATTTG GAGTTTGCTA AGGGCAGTAT TTTTCTGTCA | |
| | | AGACTATTCA AGAAGGCATT ATTTGAGATT CCTGTTCATT | |
| | | CTTGGTGTGT CTCTAACAGA TACAGTATGT ATACATTTGT | |
| | | ATAATTGTTG TTGTTGAAAG TCCAGCTTTT TTGAGGTATA | |
| | | TTTTAAATGT TTTAAGGATG CTTCTAAGGA TCAGTAGTAA | |
| | | TTTTTTTAGT TCGCACCTAA AGATGATTAC ATTGACCTCC | |
| | | CCCGACTGCT TACCAAATTA AAATGTGTCC ACGAAGTAGC | |
| | | TTTGTGATCG CAGATACATT CATAGTGAAC TCATCAGAAT | |
| | | GGCTGGTTTG CAGTACTGAA ATACTATCTT CTAGGCTGTA | |
| | | TGTAGTGCTA CAATTAGAGA AACAGAAGTC CAAGGCTGGC | |
| | | GACAGCTTGA AAAGTCTGAC AGCTTTTCTA CTTTTCCTGA | |
| | | AAATTTTAAG ACTGTGATAT CTGTCATTTT ACTGTATAGC | |
| | | TGACTGTGTA CTCAGGTATT TTATTGGTCC TTGAAAGATT | |
| | | GGTCGTTATG GATCACCCAG CCTTTCCAAG TCAGTGGCTG | |
| | | TTGTTCTGTC TTGCTGTCTG ATACGAGAGT GGGGCTTTTC | |
| | | AGTGAACTAA CCAGGGATTG TTCTTGACAT ACCTGACTTT | |
| | | TCTCACATTT GAACTTCCAC TATCATTGTA TCCATATAAC | |
| | | TTCTAGCATT TTCATGCCAT GGTAATCCAT GAGCTACACA | |
| | | TACGTAGCCC GGCACCGTGA TGCAAGTTCA TGGTATCGTG | |
| | | CATGTTCGTG GTATCATGGT ATCATTCATG CGTGTTTGAA | |
| | | TAGTTCTACA TCTAGTGCTT CTTGCCAAAA AGAATACATT | |
| | | GTTTAAATTC ACAAAATTAG CATAATTGCA GTGCTAATGA | |
| | | ATATCGGAAT ATGTGCACAG TAACATTTGG ACTATTCATT | |
| | | GGAGAGTTTA CCCATACATT TAGCAAATTG AATGGCCAAA | |
| | | ACATTTGACT CCAGTGAGGG CTCAAGTTAG ATCCCTATAG | |
| | | AAAGAGGACA CTTCATCTTA CTTAAGTCAT AGTTAAGATC | |
| | | TGTGATACGA ACCATAGATA TTGCCTGACA AAGCAGAAAT | |
| | | CACCAAGTTT CCCCCTTTTG AATTACCACC AAGAAGTGTT | |
| | | GAAACACCAA ATAGATATCA TGTTATTTTG GGCATTTGCA | |
| | | GTTTTCTTCC CTGCTGCATG TAATGTCTCA GAATCAACAT | |
| | | TCTTTTAAAA TCTAGACTAT ATTTTGAGGC AATGAATTAC | |
| | | TTATATTCAA CTTAGGCTTG TTTTGACATT CAGTAGAACT | |
| | | TTAAGTTCAA TCTAAAGGCT TCAGTCCACA TTTTTTTATA | |
| | | CGTTGTATTT TAAAAACGTT TGAAAGGAGT CTTACACCTG | |
| | | TATCATGAAA ACTGAATCCT TTTGAAATAC CACTATATGA | |
| | | AGAGAGAGAT GAAATTTAGT GAACAGAATT GAAAAGGTGC | |
| | | TCATAATTTC ACTATGCAAA CTTACCCCAG TCTCTAAAAA | |
| | | AGTAATTTAG ATTTAAAGTT CTTTGATGTA TTTGATTTTC | |
| | | TAAATCTTTA TGGTTATGAT TTGGAATAAA ATGTGCCTAA | |
| | | TCCTGTGTTA CATTCTGTTC TTAAATCTGA ATGCCTTCTC | |
| | | ATTTAATTCT GAGGAAATAT CACACAAGTG TCTTCATTGA | |
| | | CCTTGAAGAA ATGTATATAC AGTTGCCTTA TAAAACAACA | |
| | | TAAATTTAGA CCATAACTTT TATAGAGAAA GGGTTTTGTC | |
| | | AAATGTTTTC TGAAAATCTG AGTAATTCAA AGCATGCCTC | |
| | | TGCCCCTTTA ATATTTTTAA TAACCTGCAT TGTTGCTGTC | |
| | | TGCCAAATAT TAAATTGAAA TCTTCATTTC AATTTTATTA | |
| | | TCTGGAAAGG GCACTGGATT GCTCTGCAAC CAAAGAAAGC | |
| | | AATATGGAAT GAAAAAACTC ATTCACTTTT GTCTTATTTT | |
| | | CTTTTAAGGT GTATTGGCAT GTAATTTGCA TAGAGAAGGT | |
| | | CCTCTGGTTA GTCTCTCAAA TTGAGGCTGT TTAGGGAAAT | |
| | | CCTTATTCAG TTGGTGGCAG TGGTTGGTTT AAAGTAGAAG | |
| | | GAAATAAGAT CGCCTTAATA CCAGAAATGA TTAGAAGTGC | |
| | | TGATTTAGAT TCAACAAATA CCATATGTCC TTATCATTTT | |
| | | TTGTAAGAAG AAATTGGTTA AGTCCTAACT TTCAATGTGT | |
| | | ACCCAAATAC TTGTATTTAT GCTTTTGATA AAATGTATTT | |
| | | TCAGCATTAA TACACATCCG ATTATGCCTT ATTTATATAT | |
| | | GAAGAATAAA GTTACCATGT TACACTGTTA TGTCCTAAAA | |
| | | TTCAAATCAC TATTTGAGAA ACCCTCAAAT TGGTGCTTTC | |
| | | ATTATATAAT GATACATTTA GACAAAACCC CAAACTAAGC | |
| | | CATTTGAAAC AAGATTCTCT CCATTGCAGT TTGTAGCAAT | |
| | | GTTATTTCTG TGTATGTCAT GAGAAGGCTA AATATCAGTG | |
| | | TTAATTTCTT GTTTGAATCC GTGAAATCAT GCCTGTAAAG | |
| | | CCCAAACATT TGTAACAAAC TCCCTAATAA ATTTAGAGAA | |
| | | AGTCACTCTG TTACCATTTC ATTTATTTTA GTTTTATTTG | |
| | | AGAAATTAAG ACCAGAAGTT TTGCTCATGT CTTTTTCACT | |
| | | TGGAAATCTG ACTTAGTACC TAGTTAATTT GTCTCTCTCC | |
| | | CATCATTTTA AGGCGTTCGC TTCTGTTAAT CACAGATGAT | |
| | | CTGTCCTTGT GTTGTGCTGC TTGACTAAAA GCACTGTTTT | |
| | | GTTTTGTTTT GTTTTGTTTT TTAATGATAG GATACAGGGT | |
| | | GGCTTTTCAG TCTAGTAAAC ATGAGGAGGG GAGAGATTGT | |
| | | GGGTGGGTGT GGGTGTGTCC GCATGTCCCA AATACAGCAC | |
| | | ATGCCCATTT TTTTATGGTA CGAGGTTAGG GCTTATTAAT | |
| | | AAATGTATAC TAGATCTTGA AGTGTAAGTC TAAGTCTGAA | |
| | | CATTTTAAAC AAGTTGGACT TTATGGCTTT TTCAAGTTCC | |
| | | ATCAAGTAGA TACTAGTCTT AGGCTGGTAG GTGATTAACA | |
| | | ACATCATTTG GAGTACAGTG TTTGTACCAC TAGCATTCTT | |
| | | ATGTCTGTAC TTGAACGTGT AGTTAGCATT TAAGTTATGT | |
| | | TCACTCTTGT AGCATTCAGG TCTTACCATC TGGTTTCAAA | |
| | | TGCGAGAGCT TATATGAACA TTGTTTTGAA AGCATGAATG | |
| | | ATATGGCAAT ATTTTATGTC TTGATAAGGA GAGTTTTGTG | |
| | | ACATATACAA ATCTGCTGTT GATGATGAAA GTTTACAGGT | |
| | | GGATCAGAGT ATGCTGGAAC TCAGTGTGCA TAAAATTTCA | |
| | | GTCAGTGAAT ATCACTGAAC GTCATATACT ACTTGGTATG | |
| | | TGACTTTGGT TTGTGTTAAG AAAGCTTGTA TATAATATTT | |
| | | TTTGCCATAG TAAGTGAGAA ATTGTCCTTA ATCATGCCTG | |
| | | TTTGATGGTA CTAGGAAAGA AAGGGGTAGA GATTAATTCT | |
| | | TGCACAGTAT AAGCAACAGT GCAACAAACT ATGCCATTTA | |
| | | CCTTTTACCT CTTACTTGAA GGCAGAATCG CAAAACGTTT | |
| | | GAAATGGCTT TTCTAAACTA CTCTACTCTG GTGAGAGCTC | |
| | | ATTTACCACA AGAAGCCTTA TAAAAAAGTA TATTTTGTAA | |
| | | TAACCCCGTA GATACTGTAC CTAACAAAAC ATGACTCGTA | |
| | | TTAGCTCTAT AAAATATTTG TGGCTTAGGA TTTTTTTTTT | |
| | | ACATATATCT TTTTATAACT TTCCAGGAAC TAAGCACATT | |
| | | ATCTGATAAT TGTTGTAAAT TTTTTTGTCA GTGCTTTAGT | |
| | | TCAGGATGGG CAATAAATTA TTTCTAAAGG AGGTCTAAAA | |
| | | GTGGAAAGAA TGGTTTGATT TTATAAAATG AGTTGCTAGT | |
| | | TCATTACAGC TTTTTACTTT TGTACATATT TTGCAAAACA | |
| | | TTTGCCTCTT GCTATTAATA TTTGCTTTGT AAAAATTACT | |
| | | GACATTTAAT AAACATTTGT AAACAATTCA AAAAAAAAAA | |
| | | AAAAAA | |
| IL8 | NM_000584.3 | GAGGGTGCAT AAGTTCTCTA GTAGGGTGAT GATATAAAAA | 7 |
| | | GCCACCGGAG CACTCCATAA GGCACAAACT TTCAGAGACA | |
| | | GCAGAGCACA CAAGCTTCTA GGACAAGAGC CAGGAAGAAA | |
| | | CCACCGGAAG GAACCATCTC ACTGTGTGTA AACATGACTT | |
| | | CCAAGCTGGC CGTGGCTCTC TTGGCAGCCT TCCTGATTTC | |
| | | TGCAGCTCTG TGTGAAGGTG CAGTTTTGCC AAGGAGTGCT | |
| | | AAAGAACTTA GATGTCAGTG CATAAAGACA TACTCCAAAC | |
| | | CTTTCCACCC CAAATTTATC AAAGAACTGA GAGTGATTGA | |
| | | GAGTGGACCA CACTGCGCCA ACACAGAAAT TATTGTAAAG | |
| | | CTTTCTGATG GAAGAGAGCT CTGTCTGGAC CCCAAGGAAA | |
| | | ACTGGGTGCA GAGGGTTGTG GAGAAGTTTT TGAAGAGGGC | |
| | | TGAGAATTCA TAAAAAAATT CATTCTCTGT GGTATCCAAG | |
| | | AATCAGTGAA GATGCCAGTG AAACTTCAAG CAAATCTACT | |
| | | TCAACACTTC ATGTATTGTG TGGGTCTGTT GTAGGGTTGC | |
| | | CAGATGCAAT ACAAGATTCC TGGTTAAATT TGAATTTCAG | |
| | | TAAACAATGA ATAGTTTTTC ATTGTACCAT GAAATATCCA | |
| | | GAACATACTT ATATGTAAAG TATTATTTAT TTGAATCTAC | |
| | | AAAAAACAAC AAATAATTTT TAAATATAAG GATTTTCCTA | |
| | | GATATTGCAC GGGAGAATAT ACAAATAGCA AAATTGAGGC | |
| | | CAAGGGCCAA GAGAATATCC GAACTTTAAT TTCAGGAATT | |
| | | GAATGGGTTT GCTAGAATGT GATATTTGAA GCATCACATA | |
| | | AAAATGATGG GACAATAAAT TTTGCCATAA AGTCAAATTT | |
| | | AGCTGGAAAT CCTGGATTTT TTTCTGTTAA ATCTGGCAAC | |
| | | CCTAGTCTGC TAGCCAGGAT CCACAAGTCC TTGTTCCACT | |
| | | GTGCCTTGGT TTCTCCTTTA TTTCTAAGTG GAAAAAGTAT | |
| | | TAGCCACCAT CTTACCTCAC AGTGATGTTG TGAGGACATG | |
| | | TGGAAGCACT TTAAGTTTTT TCATCATAAC ATAAATTATT | |
| | | TTCAAGTGTA ACTTATTAAC CTATTTATTA TTTATGTATT | |
| | | TATTTAAGCA TCAAATATTT GTGCAAGAAT TTGGAAAAAT | |
| | | AGAAGATGAA TCATTGATTG AATAGTTATA AAGATGTTAT | |
| | | AGTAAATTTA TTTTATTTTA GATATTAAAT GATGTTTTAT | |
| | | TAGATAAATT TCAATCAGGG TTTTTAGATT AAACAAACAA | |
| | | ACAATTGGGT ACCCAGTTAA ATTTTCATTT CAGATAAACA | |
| | | ACAAATAATT TTTTAGTATA AGTACATTAT TGTTTATCTG | |
| | | AAATTTTAAT TGAACTAACA ATCCTAGTTT GATACTCCCA | |
| | | GTCTTGTCAT TGCCAGCTGT GTTGGTAGTG CTGTGTTGAA | |
| | | TTACGGAATA ATGAGTTAGA ACTATTAAAA CAGCCAAAAC | |
| | | TCCACAGTCA ATATTAGTAA TTTCTTGCTG GTTGAAACTT | |
| | | GTTTATTATG TACAAATAGA TTCTTATAAT ATTATTTAAA | |
| | | TGACTGCATT TTTAAATACA AGGCTTTATA TTTTTAACTT | |
| | | TAAGATGTTT TTATGTGCTC TCCAAATTTT TTTTACTGTT | |
| | | TCTGATTGTA TGGAAATATA AAAGTAAATA TGAAACATTT | |
| | | AAAATATAAT TTGTTGTCAA AGTAAAAAAA AAAAAAAA | |
| JMJD1C | NM_001282948.1 | CTCGTTCTCA TTTGGGTGTC TGCACTGTTG GAAGCTACTG | 8 |
| | | CACTGTGTTT CCACGCAGAC AGAGTACAGT GGGAAAGGCT | |
| | | TTCTCCAGCT GATGGATGGC TGTTCTCTTT ATTTGGAGTA | |
| | | TCTTACTGGT TATGTAAGCG ACCTCGAGAG AAATTATCCT | |
| | | GACCCAGATC GGAGGATGCA GTAGTGCTTC AGCTGCAGGA | |
| | | TTTCTTAGTC GTTAACACCA CAGTGCTGAT GACAAAGAGC | |
| | | GAGTTGTAAT CCTCATCACT GCCAGGGCTG AGCCAGAGCA | |
| | | GACAGAAAAT CGTCATTTGC TCGTTGGAGA CTTGTTAGGT | |
| | | TACAGCTCCT GCTTCGTTCT GTGGTCTTGG CTTTTCTTCC | |
| | | TATACAATGA CGCCGCTGCA AGTGAGCTCA GAAATGCTTG | |
| | | GTTTGAGAGC CATTTGCCTG TGATTTTTGA AGACTAAAGA | |
| | | AGTATTTACA CTAGATACTC TTCCAGAACA AAATAGAAAT | |
| | | TTCAGCAAAT ACTTACTGAC CTTTAAAGCC TTGTTGTGGA | |
| | | TTTCTGAGAT TTAAAGATAC TTTAAAATTC TCAAGAACAA | |
| | | AACTTTCAAA CCTCTGGTTG AAAGAAATAT ACCCAGTTCA | |
| | | GTCACTGCAG TAGAATTCCT TGTAGATAAG CAACTGGATT | |
| | | TTTTAACTGA AGATAGTGCC TTTCAGCCCT ACCAGGACGA | |
| | | CATAGACAGC CTAAACCCAG TTCTCAGGGA CAACCCGCAG | |
| | | CTTCATGAGG AAGTGAAAGT CTGGGTAAAG GAACAAAAGG | |
| | | TTCAGGAGAT TTTTATGCAA GGTCCTTATT CCTTAAATGG | |
| | | ATACAGAGTG AGAGTATATA GACAAGACTC TGCCACCCAG | |
| | | TGGTTTACTG GCATAATTAC TCATCATGAT CTCTTCACCC | |
| | | GCACCATGAT CGTTATGAAT GATCAGGTAC TAGAACCACA | |
| | | GAATGTCGAT CCTTCTATGG TTCAAATGAC CTTTCTAGAT | |
| | | GATGTTGTTC ACTCTTTGTT AAAAGGTGAA AATATTGGCA | |
| | | TTACATCACG ACGCAGGTCT CGTGCCAATC AAAACGTCAA | |
| | | CGCTGTTCAC AGCCATTATA CACGTGCCCA AGCAAATAGT | |
| | | CCCAGACCAG CAATGAACTC CCAAGCTGCT GTACCAAAAC | |
| | | AGAATACACA CCAGCAACAG CAACAAAGAA GTATCCGTCC | |
| | | AAATAAGAGG AAGGGCTCAG ATAGCAGTAT ACCAGATGAA | |
| | | GAGAAGATGA AGGAGGAAAA ATATGATTAT ATATCACGAG | |
| | | GAGAAAATCC TAAAGGTAAA AACAAACACT TGATGAATAA | |
| | | AAGAAGGAAA CCTGAGGAGG ATGAAAAGAA ACTAAATATG | |
| | | AAAAGACTTC GAACTGACAA TGTTTCAGAC TTTTCTGAGA | |
| | | GCAGTGACTC AGAAAATTCA AATAAGAGAA TAATAGATAA | |
| | | TTCCTCAGAA CAGAAGCCAG AGAATGAATT GAAAAATAAA | |
| | | AATACTTCAA AAATAAATGG AGAAGAAGGA AAACCCCATA | |
| | | ATAATGAGAA GGCAGGAGAA GAGACCCTAA AAAATAGCCA | |
| | | GCCTCCCTGG GATCAAATAC AGGAAGATAA AAAACATGAA | |
| | | GAAGCAGAGA AGCGGAAGTC TGTTGACACT CAGCTTCAAG | |
| | | AAGATATGAT TATTCATTCG TCAGAACAGT CCACAGTTTC | |
| | | TGATCATAAT TCTAATGATT TACTTCCTCA GGAATGCAAT | |
| | | ATGGATAAAA CACATACCAT GGAATTGCTA CCAAAGGAGA | |
| | | AGTTTGTATC CAGACCACCC ACACCAAAAT GTGTTATTGA | |
| | | TATTACAAAT GACACTAATT TAGAAAAGGT GGCTCAGGAA | |
| | | AACTCAAGTA CCTTTGGCCT TCAGACACTT CAGAAAATGG | |
| | | ATCCTAATGT TAGTGATTCA AAACACTCTA TTGCAAATGC | |
| | | AAAATTCTTG GAAACAGCAA AAAAAGATTC TGACCAGAGC | |
| | | TGGGTCAGTG ATGTAGTTAA AGTGGATCTA ACCCAATCAA | |
| | | GTGTTACAAA TGCTTCTTCA GGAAATGATC ACTTGAACAT | |
| | | GGAAAAAGAG AAGTATGTCT CTTACATTTC TCCTTTAAGT | |
| | | GCAGTTTCTG TCATGGAAGA TAAGCTGCAT AAGCGAAGTC | |
| | | CACCTCCAGA GACTATAAAA TCTAAACTTA ATACTTCAGT | |
| | | AGATACTCAC AAGATAAAAT CCAGCCCATC ACCTGAAGTT | |
| | | GTTAAACCCA AAATAACTCA TTCTCCTGAT TCTGTAAAGT | |
| | | CTAAGGCCAC TTATGTGAAC AGCCAAGCTA CTGGTGAAAG | |
| | | AAGATTGGCA AATAAGATAG AACATGAGCT ATCAAGATGC | |
| | | AGTTTTCATC CAATTCCTAC TCGAAGCAGT ACATTAGAAA | |
| | | CTACAAAGAG TCCTCTTATC ATTGATAAAA ATGAGCATTT | |
| | | TACAGTTTAC AGAGATCCTG CACTTATTGG GTCAGAAACA | |
| | | GGAGCTAATC ATATTTCACC TTTCCTAAGC CAGCATCCTT | |
| | | TTCCTCTTCA CTCCTCATCT CATAGAACCT GTTTAAATCC | |
| | | AGGTACCCAT CATCCTGCCT TAACTCCTGC ACCCCATTTA | |
| | | CTAGCCGGAT CATCTAGTCA AACTCCATTA CCTACCATTA | |
| | | ACACTCATCC TCTGACTAGT GGTCCACACC ATGCTGTTCA | |
| | | TCACCCTCAT TTACTTCCCA CTGTGTTACC TGGAGTGCCT | |
| | | ACTGCCTCCT TACTTGGTGG CCACCCACGA CTAGAGAGTG | |
| | | CTCATGCCAG CAGCTTGAGC CACTTAGCGC TAGCACACCA | |
| | | GCAACAACAA CAGTTGTTAC AGCACCAGTC ACCTCATCTT | |
| | | CTTGGACAAG CCCATCCTTC TGCTTCATAT AATCAGCTTG | |
| | | GACTTTATCC AATTATTTGG CAGTATCCAA ATGGAACACA | |
| | | TGCATACTCA GGACTTGGTT TGCCTTCTTC TAAGTGGGTT | |
| | | CACCCAGAAA ATGCAGTTAA TGCTGAAGCT TCATTAAGGA | |
| | | GGAATTCTCC CAGTCCTTGG CTACATCAGC CCACCCCTGT | |
| | | GACCTCAGCA GATGGTATTG GATTACTTAG TCACATTCCT | |
| | | GTCAGACCTT CCAGTGCAGA GCCTCATCGG CCTCTTAAAA | |
| | | TTACAGCCCA TTCCAGTCCA CCATTGACAA AAACTTTAGT | |
| | | AGATCATCAT AAGGAAGAAT TAGAAAGAAA AGCTTTTATG | |
| | | GAACCATTAC GGTCTGTTGC ATCCACATCA GCCAAAAATG | |
| | | ACCTGGATCT AAATAGGTCA CAGACTGGAA AAGATTGTCA | |
| | | CTTACATAGG CATTTTGTGG ATCCAGTATT AAATCAGTTA | |
| | | CAGAGGCCAC CCCAGGAGAC TGGAGAGAGG TTAAACAAAT | |
| | | ACAAAGAGGA ACACCGTCGA ATTCTTCAAG AAAGTATTGA | |
| | | TGTTGCTCCC TTTACAACTA AAATCAAGGG ACTTGAGGGT | |
| | | GAGAGAGAGA ATTATTCCAG AGTGGCATCA TCATCTTCCA | |
| | | GTCCTAAAAG CCATATCATC AAACAAGATA TGGATGTAGA | |
| | | ACGCTCAGTA TCAGATCTTT ATAAAATGAA GCACTCAGTG | |
| | | CCTCAGAGTT TACCCCAAAG TAACTATTTC ACTACATTGT | |
| | | CTAATAGTGT GGTCAATGAA CCACCAAGAT CATACCCATC | |
| | | CAAAGAAGTT TCAAATATTT ACGGTGATAA ACAGAGTAAT | |
| | | GCCCTTGCAG CGGCAGCAGC TAATCCTCAA ACTCTGACTT | |
| | | CATTTATAAC ATCTCTTTCA AAGCCTCCAC CTTTGATTAA | |
| | | ACACCAACCA GAAAGTGAAG GTTTAGTAGG CAAGATACCA | |
| | | GAACATCTTC CACATCAGAT TGCATCTCAC TCAGTAACAA | |
| | | CCTTCAGAAA TGATTGTAGG AGTCCTACCC ATTTGACAGT | |
| | | TTCTTCTACA AATACACTCC GCAGTATGCC TGCATTACAT | |
| | | AGAGCACCAG TATTTCACCC ACCAATCCAT CACAGCCTGG | |
| | | AAAGAAAGGA AGGCAGCTAT AGTAGTCTTT CCCCTCCAAC | |
| | | TTTAACTCCG GTGATGCCAG TAAATGCTGG TGGTAAAGTT | |
| | | CAAGAATCAC AGAAGCCTCC AACTCTAATA CCCGAACCAA | |
| | | AAGACTCCCA GGCAAATTTT AAGAGTTCTT CAGAACAGAG | |
| | | TTTGACGGAG ATGTGGAGAC CTAATAATAA CCTCAGCAAA | |
| | | GAGAAAACTG AATGGCATGT GGAGAAAAGC AGCGGAAAGT | |
| | | TACAGGCTGC TATGGCATCT GTCATTGTGC GTCCATCTTC | |
| | | TAGTACAAAA ACTGATAGTA TGCCAGCAAT GCAGTTAGCT | |
| | | TCTAAAGATC GAGTTAGTGA AAGATCTTCA GCTGGGGCAC | |
| | | ATAAAACAGA TTGCCTCAAA CTAGCAGAAG CCGGAGAAAC | |
| | | TGGAAGAATC ATTTTGCCAA ATGTGAATTC AGACAGTGTT | |
| | | CACACAAAAT CTGAAAAAAA CTTTCAGGCT GTCTCACAGG | |
| | | GCAGTGTTCC CAGTTCAGTC ATGTCTGCTG TAAATACGAT | |
| | | GTGTAATACC AAAACGGATG TAATCACATC TGCTGCCGAT | |
| | | ACTACCAGTG TTTCCAGCTG GGGTGGTTCA GAAGTAATTT | |
| | | CCTCTTTATC AAATACCATT TTGGCCTCTA CATCATCAGA | |
| | | ATGTGTATCT TCAAAAAGTG TCAGTCAGCC AGTGGCTCAA | |
| | | AAACAAGAAT GCAAGGTCAG CACCACAGCA CCAGTTACAT | |
| | | TAGCCAGTAG TAAGACAGGA AGTGTTGTTC AACCCAGTTC | |
| | | TGGGTTCTCA GGCACAACTG ATTTTATCCA TTTAAAAAAG | |
| | | CACAAGGCAG CATTGGCTGC AGCTCAGTAT AAAAGTAGTA | |
| | | ATGCCAGTGA GACTGAACCT AATGCTATAA AAAATCAGAC | |
| | | ACTTTCAGCC TCCCTTCCTC TGGATAGCAC TGTAATCTGT | |
| | | AGTACAATTA ACAAAGCAAA CTCTGTAGGA AATGGGCAAG | |
| | | CTTCCCAGAC AAGTCAACCA AACTACCATA CTAAACTGAA | |
| | | AAAGGCCTGG CTCACCAGAC ACTCAGAAGA AGATAAAAAT | |
| | | ACTAATAAAA TGGAAAATTC AGGGAATTCT GTATCAGAAA | |
| | | TTATTAAGCC ATGTTCTGTC AACTTAATAG CCTCTACATC | |
| | | TAGTGATATA CAAAATAGTG TAGATAGTAA GATCATAGTT | |
| | | GATAAATATG TAAAAGATGA TAAAGTCAAC AGGAGAAAAG | |
| | | CCAAAAGAAC TTATGAATCT GGCTCTGAAA GTGGAGACTC | |
| | | AGATGAAAGT GAAAGCAAGT CAGAGCAAAG GACTAAAAGA | |
| | | CAACCTAAGC CAACTTACAA AAAGAAGCAA AATGATTTGC | |
| | | AAAAGAGAAA AGGTGAAATA GAAGAAGATT TGAAACCCAA | |
| | | TGGAGTTCTC AGCAGGAGTG CCAAAGAAAG AAGTAAACTG | |
| | | AAGTTGCAAA GCAACAGTAA TACTGGCATT CCTCGTTCAG | |
| | | TATTGAAAGA TTGGCGTAAA GTCAAGAAGC TGAAGCAAAC | |
| | | TGGGGAATCC TTTTTACAGG ATGACTCCTG CTGTGAGATA | |
| | | GGGCCTAATT TACAAAAGTG TCGAGAATGT AGACTTATTC | |
| | | GCAGTAAAAA AGGAGAAGAA CCAGCTCACT CACCAGTATT | |
| | | TTGTAGATTT TACTACTTTA GACGGTTGTC ATTTAGTAAA | |
| | | AACGGAGTAG TTAGAATAGA TGGTTTCTCT TCTCCTGACC | |
| | | AATATGATGA TGAAGCTATG AGTTTGTGGA CACATGAAAA | |
| | | TTTTGAAGAT GATGAACTAG ATATAGAGAC TTCTAAATAT | |
| | | ATCTTGGATA TAATAGGTGA TAAGTTCTGT CAATTAGTAA | |
| | | CATCTGAAAA AACAGCTTTG TCCTGGGTGA AAAAGGATGC | |
| | | CAAAATTGCC TGGAAAAGAG CAGTGAGAGG AGTCCGGGAG | |
| | | ATGTGTGATG CATGTGAAGC AACATTGTTT AACATTCACT | |
| | | GGGTCTGCCA AAAATGTGGA TTTGTGGTCT GCTTAGATTG | |
| | | TTACAAGGCA AAGGAAAGGA AGAGTTCTAG AGATAAAGAA | |
| | | CTATATGCTT GGATGAAGTG TGTGAAGGGA CAGCCTCATG | |
| | | ATCACAAACA TTTAATGCCA ACCCAAATTA TACCTGGTTC | |
| | | TGTTTTGACA GATCTTCTAG ATGCCATGCA CACTCTTAGG | |
| | | GAAAAATATG GTATTAAATC CCATTGTCAT TGTACTAACA | |
| | | AACAGAATTT ACAAGTTGGA AATTTTCCTA CAATGAATGG | |
| | | TGTATCTCAA GTTTTACAGA ATGTTCTTAA TCACAGTAAT | |
| | | AAAATTTCTC TGTGCATGCC TGAGTCTCAG CAGCAAAATA | |
| | | CTCCTCCGAA GTCTGAGAAA AATGGTGGCA GCAGCCCAGA | |
| | | GAGTGATGTA GGCACAGATA ACAAGTTAAC TCCTCCAGAA | |
| | | TCCCAGTCAC CACTGCACTG GTTAGCAGAT CTTGCAGAGC | |
| | | AAAAAGCCAG AGAGGAAAAA AAAGAAAACA AAGAACTTAC | |
| | | CCTTGAAAAC CAAATTAAAG AAGAAAGAGA ACAAGACAAC | |
| | | TCTGAATCTC CAAATGGCAG AACATCACCT CTTGTGTCCC | |
| | | AGAATAATGA ACAAGGCTCA ACCTTACGGG ATTTGCTGAC | |
| | | TACAACAGCT GGAAAGCTAC GTGTGGGGTC TACAGATGCT | |
| | | GGCATTGCCT TTGCCCCAGT ATATTCAATG GGAGCCCCAA | |
| | | GTAGCAAAAG TGGACGGACT ATGCCTAACA TTCTTGATGA | |
| | | CATAATTGCT TCAGTTGTTG AAAACAAAAT TCCACCAAGT | |
| | | AAAACCTCCA AGATAAATGT AAAACCAGAG CTTAAAGAAG | |
| | | AGCCTGAAGA AAGCATAATA TCTGCAGTGG ATGAAAATAA | |
| | | TAAATTATAC AGTGATATAC CACATTCTTG GATCTGTGAG | |
| | | AAGCATATTT TATGGCTTAA GGATTATAAG AATAGCAGTA | |
| | | ATTGGAAGCT TTTCAAAGAA TGTTGGAAAC AAGGACAGCC | |
| | | TGCAGTGGTT TCTGGTGTGC ATAAGAAAAT GAACATTAGC | |
| | | CTATGGAAGG CGGAATCAAT TAGTCTTGAT TTTGGAGACC | |
| | | ACCAAGCTGA TCTCCTGAAC TGCAAAGATA GCATCATTTC | |
| | | AAATGCCAAT GTTAAGGAAT TCTGGGATGG TTTTGAAGAA | |
| | | GTTTCAAAAC GGCAGAAAAA CAAGAGTGGA GAAACAGTTG | |
| | | TTTTAAAATT GAAAGACTGG CCTTCAGGAG AAGACTTCAA | |
| | | GACTATGATG CCAGCAAGAT ACGAAGATCT TTTAAAAAGT | |
| | | CTGCCATTGC CAGAATATTG TAATCCAGAA GGAAAATTCA | |
| | | ATTTGGCCTC TCATTTGCCA GGATTTTTTG TACGTCCTGA | |
| | | TCTAGGACCC AGGTTGTGCA GTGCCTATGG TGTAGTTGCT | |
| | | GCTAAAGATC ATGATATAGG AACAACAAAT CTCCATATTG | |
| | | AAGTTTCTGA TGTTGTAAAT ATACTAGTTT ATGTTGGCAT | |
| | | AGCAAAAGGA AATGGCATTC TCTCAAAAGC AGGAATTCTC | |
| | | AAGAAATTTG AGGAAGAAGA TTTGGATGAC ATTTTAAGGA | |
| | | AAAGATTGAA GGACTCAAGT GAAATACCTG GTGCTCTGTG | |
| | | GCATATTTAT GCTGGGAAAG ATGTTGACAA GATAAGGGAA | |
| | | TTTCTTCAAA AGATTTCAAA AGAACAAGGC CTTGAAGTTC | |
| | | TACCAGAACA TGATCCAATA CGTGACCAAA GTTGGTATGT | |
| | | GAACAAAAAG CTCCGTCAAA GGCTGCTTGA AGAATATGGA | |
| | | GTCAGAACCT GTACTCTTAT TCAGTTCCTT GGTGATGCTA | |
| | | TTGTTTTGCC AGCGGGAGCA CTTCATCAGG TTCAGAATTT | |
| | | TCACAGCTGT ATTCAGGTAA CTGAAGATTT TGTGTCTCCA | |
| | | GAACATCTTG TAGAGTCATT TCATTTAACA CAGGAACTGA | |
| | | GACTTTTGAA GGAAGAAATC AATTATGATG ATAAACTACA | |
| | | GGTTAAAAAT ATTTTGTATC ATGCAGTCAA AGAAATGGTG | |
| | | AGAGCCTTGA AGATACACGA GGATGAAGTA GAGGATATGG | |
| | | AAGAAAATTA AGTGTGATCC AGTTTGATAT TTTTAGGTTG | |
| | | TTGAACTGGG ATTACTTAAC CTTGAATGAT GATATGTATG | |
| | | CACACTGACT TTAAGCTTCA TAAAACCATC AGTGCCAAGA | |
| | | AATTCTCTTT GTAGTAATTA CTTGTTACTG ACACCACAGC | |
| | | AGTATAGCAT ATGTCACAGC TCCTGTGATT CAATGTTATA | |
| | | AAACAAGCAG AATTTTAAAA GCAGCACTAT ATAGCTGTTT | |
| | | TGTATTATAG TGTATATGAT GTTTGTGAAA ATGCCAGATT | |
| | | TAAAATGATG TATTTATTTT TGGTAAAAAA TAAAAAATTC | |
| | | TATGCTATAT TGTTGATCAA GTGTAAATGT GACCTTGTAC | |
| | | AGTTTACTAA AATTACTGAT ATTTTTCACT ACATTGAGAC | |
| | | AGTTACTGTG AGAATAGGAC ACAAACACCA GCTATTGCCT | |
| | | GCATCTGGGA AATTGCTGAA TCGCACAGCA GTCATGTCAT | |
| | | AATCAGAAAA TTACTGCCAA ATAATTGTAA AATTTGTAAA | |
| | | GTATAAAGTA TATAAAGTAG ATACTAAATA CAGACACTTC | |
| | | AATATTTTGT TGAAGCTATT GACTGTACAA TTAAACATTT | |
| | | TCAAAAGGTG TAATTTATTT AAAATTGTCT CATTTTGGTA | |
| | | AAATTTATGT GAACTTTTAA AGCTAAATAT TAAACTTAAT | |
| | | ATGCTATGTA AATATATACA TATATACATT TAATGATGTA | |
| | | TTTTTTTAAA ACATTGGCTT GCTTTTGTTA AAGTGCAAGT | |
| | | GTTACATATG GCTTTGTACA TTAAAGTTGA AAGGGGTTTT | |
| | | ACATTTTCCA TTAAAAGGAC TTTATCAAAA ATTGA | |
| LARS2 | NM_015340.3 | TGACAACATG GCGGCGCCCA TGGTCCGTGG CCCGGCAGTG | 9 |
| | | CTCGCCTAAA GGTGGAGAAC GAGGAGTAGA GGAGCCGCAG | |
| | | GCCAGAGCCT GTGAGCAGAT CCAGACCTAC AGATAAAAAA | |
| | | CATTATTTAA TCTATCTGGG ATTTACTCCG GCTTATGATT | |
| | | TGAGGGCCTT CTCACCTTCT GAAGAATGGC TTCTGTTTGG | |
| | | CAGAGATTGG GTTTTTATGC CTCTCTTCTG AAAAGACAGC | |
| | | TAAATGGTGG GCCAGATGTC ATCAAGTGGG AAAGGAGAGT | |
| | | AATTCCCGGA TGTACCAGAA GCATCTACAG TGCCACGGGA | |
| | | AAGTGGACAA AAGAGTATAC ATTGCAGACA AGAAAGGATG | |
| | | TTGAGAAATG GTGGCATCAA CGAATAAAAG AACAGGCCTC | |
| | | CAAAATTTCA GAAGCTGATA AATCGAAGCC AAAATTTTAC | |
| | | GTGCTTTCCA TGTTCCCTTA TCCTTCTGGT AAGCTGCACA | |
| | | TGGGCCATGT GCGTGTCTAC ACCATCAGCG ACACCATAGC | |
| | | ACGGTTCCAG AAGATGAGAG GGATGCAGGT CATCAACCCC | |
| | | ATGGGATGGG ATGCTTTTGG ATTGCCTGCT GAAAATGCCG | |
| | | CAGTCGAGAG GAATCTACAT CCACAAAGTT GGACACAAAG | |
| | | TAATATTAAA CACATGAGGA AACAGCTTGA TCGTCTGGGC | |
| | | CTGTGTTTCA GCTGGGATAG GGAAATAACT ACGTGTTTGC | |
| | | CAGATTACTA CAAGTGGACT CAGTATCTCT TTATTAAACT | |
| | | GTATGAGGCT GGGCTGGCCT ATCAAAAGGA GGCCCTGGTT | |
| | | AACTGGGACC CAGTGGATCA AACAGTGCTT GCCAATGAGC | |
| | | AGGTGGATGA ACATGGCTGT TCATGGCGTT CTGGAGCAAA | |
| | | GGTGGAACAG AAGTACCTCA GACAATGGTT TATTAAGACA | |
| | | ACCGCTTATG CAAAGGCCAT GCAGGACGCG TTGGCAGACC | |
| | | TTCCAGAATG GTATGGAATA AAAGGCATGC AAGCCCACTG | |
| | | GATTGGGGAC TGTGTGGGCT GCCACCTGGA CTTCACATTA | |
| | | AAGGTTCATG GGCAAGCCAC GGGCGAAAAG CTGACTGCCT | |
| | | ATACGGCCAC CCCTGAAGCC ATTTATGGCA CCTCCCACGT | |
| | | GGCCATCTCG CCCAGCCACA GACTCCTACA TGGGCACAGC | |
| | | TCTCTGAAGG AAGCCTTGAG GATGGCCCTT GTCCCTGGCA | |
| | | AAGATTGCCT CACGCCTGTA ATGGCTGTGA ACATGCTTAC | |
| | | CCAGCAGGAG GTCCCTGTCG TTATTTTGGC CAAAGCTGAC | |
| | | TTGGAAGGCT CTCTGGATTC AAAAATAGGA ATTCCCAGTA | |
| | | CTAGCTCAGA GGACACCATC TTAGCCCAAA CCCTGGGCCT | |
| | | GGCCTACTCT GAAGTCATTG AAACTTTGCC AGATGGCACA | |
| | | GAGAGACTGA GCAGCTCTGC TGAGTTCACA GGTATGACCC | |
| | | GGCAGGATGC TTTTCTAGCC CTGACTCAGA AAGCCCGGGG | |
| | | GAAGAGAGTG GGTGGAGACG TGACAAGTGA TAAACTGAAA | |
| | | GACTGGCTGA TTTCACGGCA GCGGTACTGG GGCACACCAA | |
| | | TCCCCATTGT CCACTGCCCA GTCTGTGGCC CCACACCTGT | |
| | | GCCCCTGGAG GACTTGCCTG TGACCCTGCC CAACATCGCG | |
| | | TCTTTCACTG GCAAGGGAGG CCCCCCACTG GCCATGGCTT | |
| | | CAGAGTGGGT GAACTGCTCC TGCCCAAGGT GCAAGGGAGC | |
| | | AGCCAAGAGA GAGACAGACA CGATGGATAC CTTTGTTGAT | |
| | | TCTGCTTGGT ACTACTTCAG ATACACTGAC CCTCATAATC | |
| | | CACACAGCCC TTTTAACACA GCAGTGGCCG ATTACTGGAT | |
| | | GCCTGTGGAT TTGTACATTG GAGGGAAAGA ACATGCCGTC | |
| | | ATGCACTTGT TCTATGCAAG ATTCTTTAGT CATTTTTGCC | |
| | | ATGATCAAAA AATGGTTAAA CATAGGGAGC CTTTTCATAA | |
| | | GCTGCTGGCC CAAGGCCTTA TCAAGGGGCA GACATTCCGC | |
| | | CTACCATCTG GACAGTATCT ACAGAGAGAG GAAGTGGATC | |
| | | TCACAGGTTC CGTTCCTGTT CATGCAAAAA CGAAAGAGAA | |
| | | GTTAGAGGTG ACGTGGGAGA AGATGAGTAA GTCCAAACAC | |
| | | AACGGGGTGG ACCCAGAGGA AGTTGTGGAG CAGTATGGGA | |
| | | TCGACACGAT TCGGCTCTAC ATCCTTTTTG CTGCCCCTCC | |
| | | TGAGAAGGAT ATCTTGTGGG ATGTGAAAAC TGATGCTCTC | |
| | | CCTGGGGTGC TGAGATGGCA ACAACGACTG TGGACCTTGA | |
| | | CAACTCGGTT TATTGAGGCC AGGGCTTCTG GGAAGTCTCC | |
| | | CCAGCCTCAG CTGCTGAGTA ACAAGGAGAA AGCTGAGGCC | |
| | | AGGAAGCTCT GGGAGTACAA GAACTCCGTC ATCTCTCAGG | |
| | | TGACCACCCA TTTCACAGAG GACTTCTCAC TGAATTCTGC | |
| | | AATTTCTCAG CTGATGGGAC TCAGCAATGC CCTCTCGCAA | |
| | | GCCTCTCAGA GCGTCATTCT CCACAGCCCC GAGTTTGAGG | |
| | | ATGCTTTGTG TGCCCTGATG GTAATGGCTG CTCCACTGGC | |
| | | CCCTCATGTA ACCTCAGAGA TCTGGGCAGG CCTGGCGCTG | |
| | | GTGCCGAGGA AGCTCTGTGC CCACTACACT TGGGATGCCA | |
| | | GTGTGCTGCT CCAGGCATGG CCTGCTGTGG ACCCGGAGTT | |
| | | CCTGCAGCAG CCTGAGGTTG TCCAGATGGC AGTTCTGATC | |
| | | AACAATAAAG CTTGTGGCAA AATTCCTGTG CCCCAACAAG | |
| | | TTGCCCGGGA CCAGGACAAA GTCCACGAAT TTGTTCTTCA | |
| | | AAGCGAGCTG GGTGTCAGGC TTTTGCAAGG ACGAAGCATC | |
| | | AAGAAGTCCT TCCTTTCCCC GAGAACTGCC CTCATCAACT | |
| | | TCCTGGTGCA AGATTGACAG CCAGGAGGCT GCAGCTACCA | |
| | | CGAGGGCCTC TGAGGAACCT CCTTCCAGGC CTGGGATGAG | |
| | | GGGGCGATGT CTGCTGGCCC AGGGGAAGGG AAAAGACAAA | |
| | | TGTCTTGACT GTTGACCTCG GTCCTGTGGC AGACTGCAGT | |
| | | CAACAGTGTG CCTCTGTAGT GTGGCCTGGT GCTGGGGTGA | |
| | | AGGTGAGCTG GGCAAAGGAG AAATATGAGC TACTGAGGAG | |
| | | GGGGTTGGAC ATCCTGCCCC TCACCCCCCA CCCACACTGC | |
| | | AGGTAGAGGA GGCCATCTGA TCCCATGGGA AGCCATCAGA | |
| | | GACACTGCTG GTGGGAGCAG GAAGGAGCAG TGCCCCTCGA | |
| | | GCAGCCAGGA AGCCTGCGGA TCTGGGAAAT GGCTCTGCCT | |
| | | TAGGCACTTC TCGGGAATTT GAGGCCAGCC TGAGGAACTG | |
| | | CAGGACTCAG GTGCAATGTG CCAGCCACTT GGAACTGCTA | |
| | | ACTGAGCCTC CAGATGGTAG TGAATGGTCT CTTTGCCTTC | |
| | | AGGCTGGATG AGGAAGTCAT TTAGGAAATG TTCAAATAAC | |
| | | CAATATGTGG AAATGGACAC AGGGATCTTC TGAAGTTGCT | |
| | | TTGAATCAAA AGGCAGGCAG TGCTGGTTCC TCTGCCTGTG | |
| | | TCCCCACCAC TCCCCAGCTC TGTCATGCAG GCCTGTCCTC | |
| | | CCCAACCCCA GCTGGATGTG CCTCCCAGGC CTGCTGTGGT | |
| | | TCTGACACAC AGGATCCCAG GCAAGGCACC ACTTCCTCAC | |
| | | ATGAATGAGG AGCAGCAAGT CATAACCACT CCCTTGGGTA | |
| | | TACAATTTGC TGTGTAGTGA AGTGGAACCA GGCTCAGGCT | |
| | | GCTGGTCCCA ACCTCAGAGC CCCACCGCAG CCCAGTAGGG | |
| | | ATGCAGCACG CCCCAGAGGG CTCATGTGGG CCCCAGATGG | |
| | | CAATGCCACC ATTGTTGATG TGACTCCAGA GCCAGTTATT | |
| | | AGGAAGAGCA AGCTCACCAC AGAGGAGTGG AACTGAGGCC | |
| | | CCCCAGATGT TGCCTCCGGT GTCCAAGCCA CAGCGGTCTG | |
| | | GCTGTTGGGA AGATGGCCAG GAATGGACTC ATACCATTGG | |
| | | CACATTAGGC TAATCCTGGT TTTATGTGAA GTCAGCAATT | |
| | | AAGTGTTCCC ACTAGAACTG ACCTAAGCCA CTGATTAATA | |
| | | TTTAATGAGG GAAGGTAGGG GAGAATCTAG CCATTTTATA | |
| | | ATGCCAGAAA TCTATATATG TTATCTGATG CCATTTTTCT | |
| | | GAAGTAGCCT CACATGTGGT CCCCCTGCAG TTCAGCAGTT | |
| | | AACAGATGAC TTTTTTAGTG TAATAAAATG TTTATCATCT | |
| | | ATG | |
| MALAT1 | NR_002819.2 | GTAAAGGACT GGGGCCCCGC AACTGGCCTC TCCTGCCCTC | 10 |
| | | TTAAGCGCAG CGCCATTTTA GCAACGCAGA AGCCCGGCGC | |
| | | CGGGAAGCCT CAGCTCGCCT GAAGGCAGGT CCCCTCTGAC | |
| | | GCCTCCGGGA GCCCAGGTTT CCCAGAGTCC TTGGGACGCA | |
| | | GCGACGAGTT GTGCTGCTAT CTTAGCTGTC CTTATAGGCT | |
| | | GGCCATTCCA GGTGGTGGTA TTTAGATAAA ACCACTCAAA | |
| | | CTCTGCAGTT TGGTCTTGGG GTTTGGAGGA AAGCTTTTAT | |
| | | TTTTCTTCCT GCTCCGGTTC AGAAGGTCTG AAGCTCATAC | |
| | | CTAACCAGGC ATAACACAGA ATCTGCAAAA CAAAAACCCC | |
| | | TAAAAAAGCA GACCCAGAGC AGTGTAAACA CTTCTGGGTG | |
| | | TGTCCCTGAC TGGCTGCCCA AGGTCTCTGT GTCTTCGGAG | |
| | | ACAAAGCCAT TCGCTTAGTT GGTCTACTTT AAAAGGCCAC | |
| | | TTGAACTCGC TTTCCATGGC GATTTGCCTT GTGAGCACTT | |
| | | TCAGGAGAGC CTGGAAGCTG AAAAACGGTA GAAAAATTTC | |
| | | CGTGCGGGCC GTGGGGGGCT GGCGGCAACT GGGGGGCCGC | |
| | | AGATCAGAGT GGGCCACTGG CAGCCAACGG CCCCCGGGGC | |
| | | TCAGGCGGGG AGCAGCTCTG TGGTGTGGGA TTGAGGCGTT | |
| | | TTCCAAGAGT GGGTTTTCAC GTTTCTAAGA TTTCCCAAGC | |
| | | AGACAGCCCG TGCTGCTCCG ATTTCTCGAA CAAAAAAGCA | |
| | | AAACGTGTGG CTGTCTTGGG AGCAAGTCGC AGGACTGCAA | |
| | | GCAGTTGGGG GAGAAAGTCC GCCATTTTGC CACTTCTCAA | |
| | | CCGTCCCTGC AAGGCTGGGG CTCAGTTGCG TAATGGAAAG | |
| | | TAAAGCCCTG AACTATCACA CTTTAATCTT CCTTCAAAAG | |
| | | GTGGTAAACT ATACCTACTG TCCCTCAAGA GAACACAAGA | |
| | | AGTGCTTTAA GAGGTATTTT AAAAGTTCCG GGGGTTTTGT | |
| | | GAGGTGTTTG ATGACCCGTT TAAAATATGA TTTCCATGTT | |
| | | TCTTTTGTCT AAAGTTTGCA GCTCAAATCT TTCCACACGC | |
| | | TAGTAATTTA AGTATTTCTG CATGTGTAGT TTGCATTCAA | |
| | | GTTCCATAAG CTGTTAAGAA AAATCTAGAA AAGTAAAACT | |
| | | AGAACCTATT TTTAACCGAA GAACTACTTT TTGCCTCCCT | |
| | | CACAAAGGCG GCGGAAGGTG ATCGAATTCC GGTGATGCGA | |
| | | GTTGTTCTCC GTCTATAAAT ACGCCTCGCC CGAGCTGTGC | |
| | | GGTAGGCATT GAGGCAGCCA GCGCAGGGGC TTCTGCTGAG | |
| | | GGGGCAGGCG GAGCTTGAGG AAACCGCAGA TAAGTTTTTT | |
| | | TCTCTTTGAA AGATAGAGAT TAATACAACT ACTTAAAAAA | |
| | | TATAGTCAAT AGGTTACTAA GATATTGCTT AGCGTTAAGT | |
| | | TTTTAACGTA ATTTTAATAG CTTAAGATTT TAAGAGAAAA | |
| | | TATGAAGACT TAGAAGAGTA GCATGAGGAA GGAAAAGATA | |
| | | AAAGGTTTCT AAAACATGAC GGAGGTTGAG ATGAAGCTTC | |
| | | TTCATGGAGT AAAAAATGTA TTTAAAAGAA AATTGAGAGA | |
| | | AAGGACTACA GAGCCCCGAA TTAATACCAA TAGAAGGGCA | |
| | | ATGCTTTTAG ATTAAAATGA AGGTGACTTA AACAGCTTAA | |
| | | AGTTTAGTTT AAAAGTTGTA GGTGATTAAA ATAATTTGAA | |
| | | GGCGATCTTT TAAAAAGAGA TTAAACCGAA GGTGATTAAA | |
| | | AGACCTTGAA ATCCATGACG CAGGGAGAAT TGCGTCATTT | |
| | | AAAGCCTAGT TAACGCATTT ACTAAACGCA GACGAAAATG | |
| | | GAAAGATTAA TTGGGAGTGG TAGGATGAAA CAATTTGGAG | |
| | | AAGATAGAAG TTTGAAGTGG AAAACTGGAA GACAGAAGTA | |
| | | CGGGAAGGCG AAGAAAAGAA TAGAGAAGAT AGGGAAATTA | |
| | | GAAGATAAAA ACATACTTTT AGAAGAAAAA AGATAAATTT | |
| | | AAACCTGAAA AGTAGGAAGC AGAAGAAAAA AGACAAGCTA | |
| | | GGAAACAAAA AGCTAAGGGC AAAATGTACA AACTTAGAAG | |
| | | AAAATTGGAA GATAGAAACA AGATAGAAAA TGAAAATATT | |
| | | GTCAAGAGTT TCAGATAGAA AATGAAAAAC AAGCTAAGAC | |
| | | AAGTATTGGA GAAGTATAGA AGATAGAAAA ATATAAAGCC | |
| | | AAAAATTGGA TAAAATAGCA CTGAAAAAAT GAGGAAATTA | |
| | | TTGGTAACCA ATTTATTTTA AAAGCCCATC AATTTAATTT | |
| | | CTGGTGGTGC AGAAGTTAGA AGGTAAAGCT TGAGAAGATG | |
| | | AGGGTGTTTA CGTAGACCAG AACCAATTTA GAAGAATACT | |
| | | TGAAGCTAGA AGGGGAAGTT GGTTAAAAAT CACATCAAAA | |
| | | AGCTACTAAA AGGACTGGTG TAATTTAAAA AAAACTAAGG | |
| | | CAGAAGGCTT TTGGAAGAGT TAGAAGAATT TGGAAGGCCT | |
| | | TAAATATAGT AGCTTAGTTT GAAAAATGTG AAGGACTTTC | |
| | | GTAACGGAAG TAATTCAAGA TCAAGAGTAA TTACCAACTT | |
| | | AATGTTTTTG CATTGGACTT TGAGTTAAGA TTATTTTTTA | |
| | | AATCCTGAGG ACTAGCATTA ATTGACAGCT GACCCAGGTG | |
| | | CTACACAGAA GTGGATTCAG TGAATCTAGG AAGACAGCAG | |
| | | CAGACAGGAT TCCAGGAACC AGTGTTTGAT GAAGCTAGGA | |
| | | CTGAGGAGCA AGCGAGCAAG CAGCAGTTCG TGGTGAAGAT | |
| | | AGGAAAAGAG TCCAGGAGCC AGTGCGATTT GGTGAAGGAA | |
| | | GCTAGGAAGA AGGAAGGAGC GCTAACGATT TGGTGGTGAA | |
| | | GCTAGGAAAA AGGATTCCAG GAAGGAGCGA GTGCAATTTG | |
| | | GTGATGAAGG TAGCAGGCGG CTTGGCTTGG CAACCACACG | |
| | | GAGGAGGCGA GCAGGCGTTG TGCGTAGAGG ATCCTAGACC | |
| | | AGCATGCCAG TGTGCCAAGG CCACAGGGAA AGCGAGTGGT | |
| | | TGGTAAAAAT CCGTGAGGTC GGCAATATGT TGTTTTTCTG | |
| | | GAACTTACTT ATGGTAACCT TTTATTTATT TTCTAATATA | |
| | | ATGGGGGAGT TTCGTACTGA GGTGTAAAGG GATTTATATG | |
| | | GGGACGTAGG CCGATTTCCG GGTGTTGTAG GTTTCTCTTT | |
| | | TTCAGGCTTA TACTCATGAA TCTTGTCTGA AGCTTTTGAG | |
| | | GGCAGACTGC CAAGTCCTGG AGAAATAGTA GATGGCAAGT | |
| | | TTGTGGGTTT TTTTTTTTTA CACGAATTTG AGGAAAACCA | |
| | | AATGAATTTG ATAGCCAAAT TGAGACAATT TCAGCAAATC | |
| | | TGTAAGCAGT TTGTATGTTT AGTTGGGGTA ATGAAGTATT | |
| | | TCAGTTTTGT GAATAGATGA CCTGTTTTTA CTTCCTCACC | |
| | | CTGAATTCGT TTTGTAAATG TAGAGTTTGG ATGTGTAACT | |
| | | GAGGCGGGGG GGAGTTTTCA GTATTTTTTT TTGTGGGGGT | |
| | | GGGGGCAAAA TATGTTTTCA GTTCTTTTTC CCTTAGGTCT | |
| | | GTCTAGAATC CTAAAGGCAA ATGACTCAAG GTGTAACAGA | |
| | | AAACAAGAAA ATCCAATATC AGGATAATCA GACCACCACA | |
| | | GGTTTACAGT TTATAGAAAC TAGAGCAGTT CTCACGTTGA | |
| | | GGTCTGTGGA AGAGATGTCC ATTGGAGAAA TGGCTGGTAG | |
| | | TTACTCTTTT TTCCCCCCAC CCCCTTAATC AGACTTTAAA | |
| | | AGTGCTTAAC CCCTTAAACT TGTTATTTTT TACTTGAAGC | |
| | | ATTTTGGGAT GGTCTTAACA GGGAAGAGAG AGGGTGGGGG | |
| | | AGAAAATGTT TTTTTCTAAG ATTTTCCACA GATGCTATAG | |
| | | TACTATTGAC AAACTGGGTT AGAGAAGGAG TGTACCGCTG | |
| | | TGCTGTTGGC ACGAACACCT TCAGGGACTG GAGCTGCTTT | |
| | | TATCCTTGGA AGAGTATTCC CAGTTGAAGC TGAAAAGTAC | |
| | | AGCACAGTGC AGCTTTGGTT CATATTCAGT CATCTCAGGA | |
| | | GAACTTCAGA AGAGCTTGAG TAGGCCAAAT GTTGAAGTTA | |
| | | AGTTTTCCAA TAATGTGACT TCTTAAAAGT TTTATTAAAG | |
| | | GGGAGGGGCA AATATTGGCA ATTAGTTGGC AGTGGCCTGT | |
| | | TACGGTTGGG ATTGGTGGGG TGGGTTTAGG TAATTGTTTA | |
| | | GTTTATGATT GCAGATAAAC TCATGCCAGA GAACTTAAAG | |
| | | TCTTAGAATG GAAAAAGTAA AGAAATATCA ACTTCCAAGT | |
| | | TGGCAAGTAA CTCCCAATGA TTTAGTTTTT TTCCCCCCAG | |
| | | TTTGAATTGG GAAGCTGGGG GAAGTTAAAT ATGAGCCACT | |
| | | GGGTGTACCA GTGCATTAAT TTGGGCAAGG AAAGTGTCAT | |
| | | AATTTGATAC TGTATCTGTT TTCCTTCAAA GTATAGAGCT | |
| | | TTTGGGGAAG GAAAGTATTG AACTGGGGGT TGGTCTGGCC | |
| | | TACTGGGCTG ACATTAACTA CAATTATGGG AAATGCAAAA | |
| | | GTTGTTTGGA TATGGTAGTG TGTGGTTCTC TTTTGGAATT | |
| | | TTTTTCAGGT GATTTAATAA TAATTTAAAA CTACTATAGA | |
| | | AACTGCAGAG CAAAGGAAGT GGCTTAATGA TCCTGAAGGG | |
| | | ATTTCTTCTG ATGGTAGCTT TTGTATTATC AAGTAAGATT | |
| | | CTATTTTCAG TTGTGTGTAA GCAAGTTTTT TTTTAGTGTA | |
| | | GGAGAAATAC TTTTCCATTG TTTAACTGCA AAACAAGATG | |
| | | TTAAGGTATG CTTCAAAAAT TTTGTAAATT GTTTATTTTA | |
| | | AACTTATCTG TTTGTAAATT GTAACTGATT AAGAATTGTG | |
| | | ATAGTTCAGC TTGAATGTCT CTTAGAGGGT GGGCTTTTGT | |
| | | TGATGAGGGA GGGGAAACTT TTTTTTTTTC TATAGACTTT | |
| | | TTTCAGATAA CATCTTCTGA GTCATAACCA GCCTGGCAGT | |
| | | ATGATGGCCT AGATGCAGAG AAAACAGCTC CTTGGTGAAT | |
| | | TGATAAGTAA AGGCAGAAAA GATTATATGT CATACCTCCA | |
| | | TTGGGGAATA AGCATAACCC TGAGATTCTT ACTACTGATG | |
| | | AGAACATTAT CTGCATATGC CAAAAAATTT TAAGCAAATG | |
| | | AAAGCTACCA ATTTAAAGTT ACGGAATCTA CCATTTTAAA | |
| | | GTTAATTGCT TGTCAAGCTA TAACCACAAA AATAATGAAT | |
| | | TGATGAGAAA TACAATGAAG AGGCAATGTC CATCTCAAAA | |
| | | TACTGCTTTT ACAAAAGCAG AATAAAAGCG AAAAGAAATG | |
| | | AAAATGTTAC ACTACATTAA TCCTGGAATA AAAGAAGCCG | |
| | | AAATAAATGA GAGATGAGTT GGGATCAAGT GGATTGAGGA | |
| | | GGCTGTGCTG TGTGCCAATG TTTCGTTTGC CTCAGACAGG | |
| | | TATCTCTTCG TTATCAGAAG AGTTGCTTCA TTTCATCTGG | |
| | | GAGCAGAAAA CAGCAGGCAG CTGTTAACAG ATAAGTTTAA | |
| | | CTTGCATCTG CAGTATTGCA TGTTAGGGAT AAGTGCTTAT | |
| | | TTTTAAGAGC TGTGGAGTTC TTAAATATCA ACCATGGCAC | |
| | | TTTCTCCTGA CCCCTTCCCT AGGGGATTTC AGGATTGAGA | |
| | | AATTTTTCCA TCGAGCCTTT TTAAAATTGT AGGACTTGTT | |
| | | CCTGTGGGCT TCAGTGATGG GATAGTACAC TTCACTCAGA | |
| | | GGCATTTGCA TCTTTAAATA ATTTCTTAAA AGCCTCTAAA | |
| | | GTGATCAGTG CCTTGATGCC AACTAAGGAA ATTTGTTTAG | |
| | | CATTGAATCT CTGAAGGCTC TATGAAAGGA ATAGCATGAT | |
| | | GTGCTGTTAG AATCAGATGT TACTGCTAAA ATTTACATGT | |
| | | TGTGATGTAA ATTGTGTAGA AAACCATTAA ATCATTCAAA | |
| | | ATAATAAACT ATTTTTATTA GAGAATGTAT ACTTTTAGAA | |
| | | AGCTGTCTCC TTATTTAAAT AAAATAGTGT TTGTCTGTAG | |
| | | TTCAGTGTTG GGGCAATCTT GGGGGGGATT CTTCTCTAAT | |
| | | CTTTCAGAAA CTTTGTCTGC GAACACTCTT TAATGGACCA | |
| | | GATCAGGATT TGAGCGGAAG AACGAATGTA ACTTTAAGGC | |
| | | AGGAAAGACA AATTTTATTC TTCATAAAGT GATGAGCATA | |
| | | TAATAATTCC AGGCACATGG CAATAGAGGC CCTCTAAATA | |
| | | AGGAATAAAT AACCTCTTAG ACAGGTGGGA GATTATGATC | |
| | | AGAGTAAAAG GTAATTACAC ATTTTATTTC CAGAAAGTCA | |
| | | GGGGTCTATA AATTGACAGT GATTAGAGTA ATACTTTTTC | |
| | | ACATTTCCAA AGTTTGCATG TTAACTTTAA ATGCTTACAA | |
| | | TCTTAGAGTG GTAGGCAATG TTTTACACTA TTGACCTTAT | |
| | | ATAGGGAAGG GAGGGGGTGC CTGTGGGGTT TTAAAGAATT | |
| | | TTCCTTTGCA GAGGCATTTC ATCCTTCATG AAGCCATTCA | |
| | | GGATTTTGAA TTGCATATGA GTGCTTGGCT CTTCCTTCTG | |
| | | TTCTAGTGAG TGTATGAGAC CTTGCAGTGA GTTTATCAGC | |
| | | ATACTCAAAA TTTTTTTCCT GGAATTTGGA GGGATGGGAG | |
| | | GAGGGGGTGG GGCTTACTTG TTGTAGCTTT TTTTTTTTTT | |
| | | ACAGACTTCA CAGAGAATGC AGTTGTCTTG ACTTCAGGTC | |
| | | TGTCTGTTCT GTTGGCAAGT AAATGCAGTA CTGTTCTGAT | |
| | | CCCGCTGCTA TTAGAATGCA TTGTGAAACG ACTGGAGTAT | |
| | | GATTAAAAGT TGTGTTCCCC AATGCTTGGA GTAGTGATTG | |
| | | TTGAAGGAAA AAATCCAGCT GAGTGATAAA GGCTGAGTGT | |
| | | TGAGGAAATT TCTGCAGTTT TAAGCAGTCG TATTTGTGAT | |
| | | TGAAGCTGAG TACATTTTGC TGGTGTATTT TTAGGTAAAA | |
| | | TGCTTTTTGT TCATTTCTGG TGGTGGGAGG GGACTGAAGC | |
| | | CTTTAGTCTT TTCCAGATGC AACCTTAAAA TCAGTGACAA | |
| | | GAAACATTCC AAACAAGCAA CAGTCTTCAA GAAATTAAAC | |
| | | TGGCAAGTGG AAATGTTTAA ACAGTTCAGT GATCTTTAGT | |
| | | GCATTGTTTA TGTGTGGGTT TCTCTCTCCC CTCCCTTGGT | |
| | | CTTAATTCTT ACATGCAGGA ACACTCAGCA GACACACGTA | |
| | | TGCGAAGGGC CAGAGAAGCC AGACCCAGTA AGAAAAAATA | |
| | | GCCTATTTAC TTTAAATAAA CCAAACATTC CATTTTAAAT | |
| | | GTGGGGATTG GGAACCACTA GTTCTTTCAG ATGGTATTCT | |
| | | TCAGACTATA GAAGGAGCTT CCAGTTGAAT TCACCAGTGG | |
| | | ACAAAATGAG GAAAACAGGT GAACAAGCTT TTTCTGTATT | |
| | | TACATACAAA GTCAGATCAG TTATGGGACA ATAGTATTGA | |
| | | ATAGATTTCA GCTTTATGCT GGAGTAACTG GCATGTGAGC | |
| | | AAACTGTGTT GGCGTGGGGG TGGAGGGGTG AGGTGGGCGC | |
| | | TAAGCCTTTT TTTAAGATTT TTCAGGTACC CCTCACTAAA | |
| | | GGCACCGAAG GCTTAAAGTA GGACAACCAT GGAGCCTTCC | |
| | | TGTGGCAGGA GAGACAACAA AGCGCTATTA TCCTAAGGTC | |
| | | AAGAGAAGTG TCAGCCTCAC CTGATTTTTA TTAGTAATGA | |
| | | GGACTTGCCT CAACTCCCTC TTTCTGGAGT GAAGCATCCG | |
| | | AAGGAATGCT TGAAGTACCC CTGGGCTTCT CTTAACATTT | |
| | | AAGCAAGCTG TTTTTATAGC AGCTCTTAAT AATAAAGCCC | |
| | | AAATCTCAAG CGGTGCTTGA AGGGGAGGGA AAGGGGGAAA | |
| | | GCGGGCAACC ACTTTTCCCT AGCTTTTCCA GAAGCCTGTT | |
| | | AAAAGCAAGG TCTCCCCACA AGCAACTTCT CTGCCACATC | |
| | | GCCACCCCGT GCCTTTTGAT CTAGCACAGA CCCTTCACCC | |
| | | CTCACCTCGA TGCAGCCAGT AGCTTGGATC CTTGTGGGCA | |
| | | TGATCCATAA TCGGTTTCAA GGTAACGATG GTGTCGAGGT | |
| | | CTTTGGTGGG TTGAACTATG TTAGAAAAGG CCATTAATTT | |
| | | GCCTGCAAAT TGTTAACAGA AGGGTATTAA AACCACAGCT | |
| | | AAGTAGCTCT ATTATAATAC TTATCCAGTG ACTAAAACCA | |
| | | ACTTAAACCA GTAAGTGGAG AAATAACATG TTCAAGAACT | |
| | | GTAATGCTGG GTGGGAACAT GTAACTTGTA GACTGGAGAA | |
| | | GATAGGCATT TGAGTGGCTG AGAGGGCTTT TGGGTGGGAA | |
| | | TGCAAAAATT CTCTGCTAAG ACTTTTTCAG GTGAACATAA | |
| | | CAGACTTGGC CAAGCTAGCA TCTTAGCGGA AGCTGATCTC | |
| | | CAATGCTCTT CAGTAGGGTC ATGAAGGTTT TTCTTTTCCT | |
| | | GAGAAAACAA CACGTATTGT TTTCTCAGGT TTTGCTTTTT | |
| | | GGCCTTTTTC TAGCTTAAAA AAAAAAAAAG CAAAAGATGC | |
| | | TGGTGGTTGG CACTCCTGGT TTCCAGGACG GGGTTCAAAT | |
| | | CCCTGCGGCG TCTTTGCTTT GACTACTAAT CTGTCTTCAG | |
| | | GACTCTTTCT GTATTTCTCC TTTTCTCTGC AGGTGCTAGT | |
| | | TCTTGGAGTT TTGGGGAGGT GGGAGGTAAC AGCACAATAT | |
| | | CTTTGAACTA TATACATCCT TGATGTATAA TTTGTCAGGA | |
| | | GCTTGACTTG ATTGTATATT CATATTTACA CGAGAACCTA | |
| | | ATATAACTGC CTTGTCTTTT TCAGGTAATA GCCTGCAGCT | |
| | | GGTGTTTTGA GAAGCCCTAC TGCTGAAAAC TTAACAATTT | |
| | | TGTGTAATAA AAATGGAGAA GCTCTAAA | |
| MBNL1 | NM_001314057.1 | CTTGCCGGCT TCCTTGCAAA GCCCGGTGCA AGGGCCTCTT | 11 |
| | | TCAAAATGAA CCCACTGGTG TGCCTAGCAG TCGGTAGAAG | |
| | | AAGCGGGAGG GCGTCCGGTC TGCACGCCCG CCGCGAGGTT | |
| | | ACAATGCTGA ACGCATGAGA TGGAAGATAC CAACGGGAGG | |
| | | CCGAGGGGAT CCACGGCGCC CGCGCGGGCT CCGGCTTCCT | |
| | | CCTGCTCTCG GCGCCGCTGG GCGACCGCCC ATGACCCGCT | |
| | | CTTGCGGGCT CTGTCCGGTT GACAGGCGAC CCTGTGGCCC | |
| | | GGGGAAGCGC GGGAGGGCGC CGGCGGAAAG TTGAAGAGCG | |
| | | TTTTTCTCGC CGCCGCGTGC ATTAGGAGCT CGACGAGTCC | |
| | | GCCCTGGGCT TCCTGGTGGG GCTGGGCGGG CGGGGGAGGG | |
| | | GCCGCGCAGC AGCAGCGGAA GCCAGACCTC GGCGATAAGA | |
| | | GGCTGCACAG CGACATGCAA CAGTCTTTTC ACTGCAGCTG | |
| | | AATGAGTTGT GGCGCCCACA ATGCTCCCAT GACAAGGAGC | |
| | | TGACAAGTTC CATTTTCCGT CGCGGGCATC TTGGAATCAT | |
| | | GACTCCCACA ATGCCTTGGG CACTTGGTCG ACAGTGGGGC | |
| | | CGCCTCTGAA AAAAAAATGT GAGAGCAGTC ACTCAGGAAA | |
| | | TGTTGTTTAA GGGGAACCTT CTGGATCCTT TTCATGGCAC | |
| | | CATGGCAAGA AGAAGCTGTA TCTTATCTAT GGAAGATAAA | |
| | | GCATGGAGTT GGCTAATGGA TGCTGATAGG ACCATCTAGT | |
| | | TGCAGGAAAA CAAGCTCAGG GCTCCCACTG ATTCTACATT | |
| | | ATGGGCCGTT GCTCCAGGGA GAACTGCAAA TATCTTCATC | |
| | | CACCCCCACA TTTAAAAACG CAGTTGGAGA TAAATGGACG | |
| | | CAATAACTTG ATTCAGCAGA AGAACATGGC CATGTTGGCC | |
| | | CAGCAAATGC AACTAGCCAA TGCCATGATG CCTGGTGCCC | |
| | | CATTACAACC CGTGCCAATG TTTTCAGTTG CACCAAGCTT | |
| | | AGCCACCAAT GCATCAGCAG CCGCCTTTAA TCCCTATCTG | |
| | | GGACCTGTTT CTCCAAGCCT GGTCCCGGCA GAGATCTTGC | |
| | | CGACTGCACC AATGTTGGTT ACAGGGAATC CGGGTGTCCC | |
| | | TGTACCTGCA GCTGCTGCAG CTGCTGCACA GAAATTAATG | |
| | | CGAACAGACA GACTTGAGGT ATGTCGAGAG TACCAACGTG | |
| | | GCAATTGCAA CCGAGGAGAA AATGATTGTC GGTTTGCTCA | |
| | | TCCTGCTGAC AGCACAATGA TTGACACCAA TGACAACACA | |
| | | GTCACTGTGT GTATGGATTA CATCAAAGGG AGATGCTCTC | |
| | | GGGAAAAGTG CAAATACTTT CATCCCCCTG CACATTTGCA | |
| | | AGCCAAGATC AAGGCTGCCC AATACCAGGT CAACCAGGCT | |
| | | GCAGCTGCAC AGGCTGCAGC CACCGCAGCT GCCATGACTC | |
| | | AGTCGGCTGT CAAATCACTG AAGCGACCCC TCGAGGCAAC | |
| | | CTTTGACCTG GGAATTCCTC AAGCTGTACT TCCCCCATTA | |
| | | CCAAAGAGGC CTGCTCTTGA AAAAACCAAC GGTGCCACCG | |
| | | CAGTCTTTAA CACTGGTATT TTCCAATACC AACAGGCTCT | |
| | | AGCCAACATG CAGTTACAAC AGCATACAGC ATTTCTCCCA | |
| | | CCAGGCTCAA TATTGTGCAT GACACCCGCT ACAAGTGTTG | |
| | | TTCCCATGGT GCACGGTGCT ACGCCAGCCA CTGTGTCCGC | |
| | | AGCAACAACA TCTGCCACAA GTGTTCCCTT CGCTGCAACA | |
| | | GCCACAGCCA ACCAGATACC CATAATATCT GCCGAACATC | |
| | | TGACTAGCCA CAAGTATGTT ACCCAGATGT AGAATTTTCA | |
| | | TCACTAAACA ATCATGCTAA AGAGGAAAGG ACAGTGTGCT | |
| | | TGGTTAGAGT AAAGGACGAG GTCATTAGCC ATATTGTATA | |
| | | TATCGTCAAG CAACACACAC AAAAGTTCCT CAGCCACAAG | |
| | | ACATCCACAT ATTGCATGTT AACCAGAAGA AAAGACAACA | |
| | | TTTTCCGGAA ATCCACTGCA CACTGTTGCC TATACACTTT | |
| | | GTACATTTAA TTGATATTTG TGCTGAGGTG ATATTCCTGT | |
| | | CTAAAAGAAC AACATTGTCT TTCTTTTCTA GCACAGAGTT | |
| | | ATGCATTCAA AGATGCATAC CTAGTTAGTT TCCTATATAT | |
| | | TCATGCCATC TTGAAAAGAC AGACTATGGT GTAACCATGA | |
| | | TTCTATTATG TATTGGTACG TCTGTAGACC AAGATATAAT | |
| | | TTTTTAAAAA TAAGTTTATT TCTTTCAAGG TTTACAAATA | |
| | | ACAAAGGTGC ACCTTGTATT TAAAATTGCC ATTATAGATG | |
| | | AGAGCGTGCA TGCACAGTCA TTTTTGTTTA AGAGTAATAT | |
| | | TTTTAATGTA ATAGATTGTA AGACGTGGTG AGGGAGGGAT | |
| | | CTGACAGAGA TGAATGTGCC AAGCAAAACC ACAACTGTGT | |
| | | ATATTTTAAA GCACATCATG GCTTTAAGTA CCATGTTGTT | |
| | | AAGGATTCTC ATGAAGTGCC ATAGACTGTA CATCAAATTA | |
| | | GAGTATTATT TCTTCAGTGT TATTGTTTTC AGAGCCACAT | |
| | | TTTGTTGCAT ATTTGCTAGT ACTAATCAGT CAAAGGGCAC | |
| | | CATTCTTTTT TTTTTTTTTT GAAACCAAAG CTGTCTCAGA | |
| | | AATGGCCAAT TTAACTTTAC AGTAACAATA GACAGCACAA | |
| | | CACAAACTCT CTCAATACAG ATAAACTCAC ACATACTGGA | |
| | | GATATATATA TAATAGATAT ATATAAAATT ATTTTAATGC | |
| | | ATTGTAGTGT AATATTTATG CATACTATAC TGTATAACAT | |
| | | GTTATTCAAA AGGGATTGCC ATTTCTGAGA CACAGTAACA | |
| | | AAAAAATGAG GAAATTATTT TGCTTCTATT TATAGCCTCT | |
| | | GTCAAAAGTC AAAAGACTAT AAATGCTTTG CAAAAATGGT | |
| | | TTCACGTTTG CTTAAATGCT TCATCACAGT CACATTCAAA | |
| | | ATAGTGACTC TAAACAAAGA AGAAAGCAGC ACTGTCATCA | |
| | | GATGCATGAT AAACCAAAAT ATGAAAATGG GAAATGTTTA | |
| | | ATTAACCTAG TAATTGGGTG GGTTAAGTAC ATGGGTGAAT | |
| | | TTTATATGTG ATTTTTGTTT TGTTTTGTTT TGTTCAGATT | |
| | | AACTGCTTAT AGCCTTAGAA AGCCTTTTAC AAAATTAAAA | |
| | | AAAAAATAGA TGTGCATTCA GTTTTTAAGA ATGGAATCAT | |
| | | CCAAAGGAAT TCCTTTTTTT GAGGTTTGGA TGTTGCAGCT | |
| | | AGTAAAGGAT ATTTTTGCTC TGTTCAGCAG TTCTAAAAAT | |
| | | TGCTGAAGTA GGGGCCAGGT CACTGGTAGT TATAGTATGG | |
| | | AATGGGAGAA GTGAAAGTTC AGTTATAGAA CTTTCCATAC | |
| | | TTCCAAGTTT ACTGCAAGTT TTTATGCTTG AGAGAGATGC | |
| | | TTTCTAATAT AAGACTGATG TGTTGATTTT ACTGATTGTA | |
| | | CTGTACATCT ATTAAAGCCT TAGATTATTA CATTACGGGT | |
| | | TGGAACCCAT ACCAATGTAA TTTCAATCGT GTTAAGAAAG | |
| | | TAATGGTGAC TTCACATGTT ATTGTAGTTA GTTACATTAT | |
| | | AGAATATTAC TTATTTTTCT TGTTAAAATG TAGTTTTTCA | |
| | | TTTCCTACAT TTATTAGATT TTCATTTTCT ATTAACAATT | |
| | | GAATACCATT TCAGTTTATA GACTTGTTTT ATTAGATTTT | |
| | | ACCAATGAAT TTTTCAAAAT ACAAAAAAAA GTAGTTTTTC | |
| | | CTTCATAACA TACTCAGTTT TGAATTACAT GTAGTGTCAC | |
| | | ATGAATATTC GTATTGTTAA CTAAATGATT TATATTTTAC | |
| | | TGATTTAATA TTACAGTGTA AGAATGTCAG TCATTGTTAG | |
| | | TTCTTGTCTA GTTTTCATTA AAAGAACAAA GATCTTTTAT | |
| | | ATGGATATCT TATAAATATA TAATCATTGC TAAGTAAGAA | |
| | | GTTAAGTTGT TGCTATCGCA ACAATCCTGG CAGACAATTG | |
| | | AGTAATATTT TGATGATTTA TTTTGTTTGT AATTAGTTAT | |
| | | TATAAGAAGA TCTAGATCCT AGATATTAGA ATAAAATTTA | |
| | | TTTTCTACTG TATCCATTTC AAATGTTAAA ATATTGTTTA | |
| | | ATATTTTTGA AATCCCTGAG TATCAGGCCT TGTTATAAAT | |
| | | AAGCTGCATA ATCAATAAAT AGAACAAGGG ACTTTTTGTT | |
| | | GATAATCCAA ATACTCAAAG TTTACGTAAT GAAAATTATA | |
| | | GCGTGTGTGC AAACTCTTGA GGGTTGATTA TGCTGCAATT | |
| | | TAGCATGTTG GAACGTCTAG GGAGAAGGTT GACTTTTTGC | |
| | | ACTTCTGTAT ATAGTCAAAA GAGAGAAACC TGTATAATAG | |
| | | TAAGATCTTA TTTTGAATAA AAACGTCTAT AATTACAAGG | |
| | | AGTTTTGTTA AGGCTAATAC AATGACAGAC TGAGCAAAAT | |
| | | TGCTTGCAAA AGTGGCACAG AGTTAGCACT CCATACCCCT | |
| | | TCAAACATGT TGCTTTGCTT TCTTGTGGAC AGCTTGTAGT | |
| | | TTGCCAGGAT TTTTTCAGCT GGAAAGATAC GCCATCCTTT | |
| | | CAAACCCTCA TGACTGACAA AAACTCCATG GGGCCAAATC | |
| | | TGCCTGAAGA TCATTACCAA AAATAGCAGG TACTTCTACC | |
| | | ATTAAGGTGA AATCATGGAT CAGATATTCC TTACATTTTT | |
| | | CAAAACTACT GCATGTTTAA AACTTCAACA AAAAAAGAGA | |
| | | GAAAGAACTA TACTAAGAAC ATATATTATT CAGATCAGTT | |
| | | TCTGCCAATT TCAGTGGTTT ATTGTTCACA AAAAAATCTT | |
| | | CAAAACAAGT ATTGACTTTC ACAAAATTTA AATCATAAAC | |
| | | AGGCAAACCA AACAGCACAC TGTAGCTATA GTTGTTATGT | |
| | | GATTGTTTTT TAATTGCTGT AGGATCCTGT TCTTTCAGCA | |
| | | GGTGAAAAAT AAAACGCAGT TCAAATTTCA TGGTTTTAAT | |
| | | TTTCAACTCA GAAGCACTCA AAAATGCAAA ATGTGATAAT | |
| | | GGGCACTTGT TTAAAAGAAT TAGTGTATCC AGCCTTCACT | |
| | | CCAGCTGGTT AAAAATGTTG CACTTATCAG CAACCCTACC | |
| | | ACTTTCATCT GCTGAAAGGA CAAATGTGCT TGGTTTTACT | |
| | | ATTATGTAAT CACAACTTAC TTTCTGCTTG TAGTTGCTTA | |
| | | AAATTATGTA TTTTGTCTTG GGCTGCAATT TGTTTTATGC | |
| | | TTATTTTATT ATTACTGCAG TAGTTGACTT TGCTGTATGG | |
| | | AAAAATAAAG TGAAATTGCC CTAATAAAAC TTCTCTTTCT | |
| | | TAAGTAAAAA AAAAAAAAAA AAAAAA | |
| MCL1 | NM_001197320.1 | GCGCAACCCT CCGGAAGCTG CCGCCCCTTT CCCCTTTTAT | 12 |
| | | GGGAATACTT TTTTTAAAAA AAAAGAGTTC GCTGGCGCCA | |
| | | CCCCGTAGGA CTGGCCGCCC TAAAACCGTG ATAAAGGAGC | |
| | | TGCTCGCCAC TTCTCACTTC CGCTTCCTTC CAGTAAGGAG | |
| | | TCGGGGTCTT CCCCAGTTTT CTCAGCCAGG CGGCGGCGGC | |
| | | GACTGGCAAT GTTTGGCCTC AAAAGAAACG CGGTAATCGG | |
| | | ACTCAACCTC TACTGTGGGG GGGCCGGCTT GGGGGCCGGC | |
| | | AGCGGCGGCG CCACCCGCCC GGGAGGGCGA CTTTTGGCCA | |
| | | CCGGCGCCAA GGACACAAAG CCAATGGGCA GGTCTGGGGC | |
| | | CACCAGCAGG AAGGCGCTGG AGACCTTACG ACGGGTTGGG | |
| | | GATGGCGTGC AGCGCAACCA CGAGACGGCC TTCCAAGGCA | |
| | | TGCTTCGGAA ACTGGACATC AAAAACGAAG ACGATGTGAA | |
| | | ATCGTTGTCT CGAGTGATGA TCCATGTTTT CAGCGACGGC | |
| | | GTAACAAACT GGGGCAGGAT TGTGACTCTC ATTTCTTTTG | |
| | | GTGCCTTTGT GGCTAAACAC TTGAAGACCA TAAACCAAGA | |
| | | AAGCTGCATC GAACCATTAG CAGAAAGTAT CACAGACGTT | |
| | | CTCGTAAGGA CAAAACGGGA CTGGCTAGTT AAACAAAGAG | |
| | | GCTGGGATGG GTTTGTGGAG TTCTTCCATG TAGAGGACCT | |
| | | AGAAGGTGGC ATCAGGAATG TGCTGCTGGC TTTTGCAGGT | |
| | | GTTGCTGGAG TAGGAGCTGG TTTGGCATAT CTAATAAGAT | |
| | | AGCCTTACTG TAAGTGCAAT AGTTGACTTT TAACCAACCA | |
| | | CCACCACCAC CAAAACCAGT TTATGCAGTT GGACTCCAAG | |
| | | CTGTAACTTC CTAGAGTTGC ACCCTAGCAA CCTAGCCAGA | |
| | | AAAGCAAGTG GCAAGAGGAT TATGGCTAAC AAGAATAAAT | |
| | | ACATGGGAAG AGTGCTCCCC ATTGATTGAA GAGTCACTGT | |
| | | CTGAAAGAAG CAAAGTTCAG TTTCAGCAAC AAACAAACTT | |
| | | TGTTTGGGAA GCTATGGAGG AGGACTTTTA GATTTAGTGA | |
| | | AGATGGTAGG GTGGAAAGAC TTAATTTCCT TGTTGAGAAC | |
| | | AGGAAAGTGG CCAGTAGCCA GGCAAGTCAT AGAATTGATT | |
| | | ACCCGCCGAA TTCATTAATT TACTGTAGTG TTAAGAGAAG | |
| | | CACTAAGAAT GCCAGTGACC TGTGTAAAAG TTACAAGTAA | |
| | | TAGAACTATG ACTGTAAGCC TCAGTACTGT ACAAGGGAAG | |
| | | CTTTTCCTCT CTCTAATTAG CTTTCCCAGT ATACTTCTTA | |
| | | GAAAGTCCAA GTGTTCAGGA CTTTTATACC TGTTATACTT | |
| | | TGGCTTGGTT TCCATGATTC TTACTTTATT AGCCTAGTTT | |
| | | ATCACCAATA ATACTTGACG GAAGGCTCAG TAATTAGTTA | |
| | | TGAATATGGA TATCCTCAAT TCTTAAGACA GCTTGTAAAT | |
| | | GTATTTGTAA AAATTGTATA TATTTTTACA GAAAGTCTAT | |
| | | TTCTTTGAAA CGAAGGAAGT ATCGAATTTA CATTAGTTTT | |
| | | TTTCATACCC TTTTGAACTT TGCAACTTCC GTAATTAGGA | |
| | | ACCTGTTTCT TACAGCTTTT CTATGCTAAA CTTTGTTCTG | |
| | | TTCAGTTCTA GAGTGTATAC AGAACGAATT GATGTGTAAC | |
| | | TGTATGCAGA CTGGTTGTAG TGGAACAAAT CTGATAACTA | |
| | | TGCAGGTTTA AATTTTCTTA TCTGATTTTG GTAAGTATTC | |
| | | CTTAGATAGG TTTTTCTTTG AAAACCTGGG ATTGAGAGGT | |
| | | TGATGAATGG AAATTCTTTC ACTTCATTAT ATGCAAGTTT | |
| | | TCAATAATTA GGTCTAAGTG GAGTTTTAAG GTTACTGATG | |
| | | ACTTACAAAT AATGGGCTCT GATTGGGCAA TACTCATTTG | |
| | | AGTTCCTTCC ATTTGACCTA ATTTAACTGG TGAAATTTAA | |
| | | AGTGAATTCA TGGGCTCATC TTTAAAGCTT TTACTAAAAG | |
| | | ATTTTCAGCT GAATGGAACT CATTAGCTGT GTGCATATAA | |
| | | AAAGATCACA TCAGGTGGAT GGAGAGACAT TTGATCCCTT | |
| | | GTTTGCTTAA TAAATTATAA AATGATGGCT TGGAAAAGCA | |
| | | GGCTAGTCTA ACCATGGTGC TATTATTAGG CTTGCTTGTT | |
| | | ACACACACAG GTCTAAGCCT AGTATGTCAA TAAAGCAAAT | |
| | | ACTTACTGTT TTGTTTCTAT TAATGATTCC CAAACCTTGT | |
| | | TGCAAGTTTT TGCATTGGCA TCTTTGGATT TCAGTCTTGA | |
| | | TGTTTGTTCT ATCAGACTTA ACCTTTTATT TCCTGTCCTT | |
| | | CCTTGAAATT GCTGATTGTT CTGCTCCCTC TACAGATATT | |
| | | TATATCAATT CCTACAGCTT TCCCCTGCCA TCCCTGAACT | |
| | | CTTTCTAGCC CTTTTAGATT TTGGCACTGT GAAACCCCTG | |
| | | CTGGAAACCT GAGTGACCCT CCCTCCCCAC CAAGAGTCCA | |
| | | CAGACCTTTC ATCTTTCACG AACTTGATCC TGTTAGCAGG | |
| | | TGGTAATACC ATGGGTGCTG TGACACTAAC AGTCATTGAG | |
| | | AGGTGGGAGG AAGTCCCTTT TCCTTGGACT GGTATCTTTT | |
| | | CAACTATTGT TTTATCCTGT CTTTGGGGGC AATGTGTCAA | |
| | | AAGTCCCCTC AGGAATTTTC AGAGGAAAGA ACATTTTATG | |
| | | AGGCTTTCTC TAAAGTTTCC TTTGTATAGG AGTATGCTCA | |
| | | CTTAAATTTA CAGAAAGAGG TGAGCTGTGT TAAACCTCAG | |
| | | AGTTTAAAAG CTACTGATAA ACTGAAGAAA GTGTCTATAT | |
| | | TGGAACTAGG GTCATTTGAA AGCTTCAGTC TCGGAACATG | |
| | | ACCTTTAGTC TGTGGACTCC ATTTAAAAAT AGGTATGAAT | |
| | | AAGATGACTA AGAATGTAAT GGGGAAGAAC TGCCCTGCCT | |
| | | GCCCATCTCA GAGCCATAAG GTCATCTTTG CTAGAGCTAT | |
| | | TTTTACCTAT GTATTTATCG TTCTTGATCA TAAGCCGCTT | |
| | | ATTTATATCA TGTATCTCTA AGGACCTAAA AGCACTTTAT | |
| | | GTAGTTTTTA ATTAATCTTA AGATCTGGTT ACGGTAACTA | |
| | | AAAAAGCCTG TCTGCCAAAT CCAGTGGAAA CAAGTGCATA | |
| | | GATGTGAATT GGTTTTTAGG GGCCCCACTT CCCAATTCAT | |
| | | TAGGTATGAC TGTGGAAATA CAGACAAGGA TCTTAGTTGA | |
| | | TATTTTGGGC TTGGGGCAGT GAGGGCTTAG GACACCCCAA | |
| | | GTGGTTTGGG AAAGGAGGAG GGGAGTGGTG GGTTTATAGG | |
| | | GGGAGGAGGA GGCAGGTGGT CTAAGTGCTG ACTGGCTACG | |
| | | TAGTTCGGGC AAATCCTCCA AAAGGGAAAG GGAGGATTTG | |
| | | CTTAGAAGGA TGGCGCTCCC AGTGACTACT TTTTGACTTC | |
| | | TGTTTGTCTT ACGCTTCTCT CAGGGAAAAA CATGCAGTCC | |
| | | TCTAGTGTTT CATGTACATT CTGTGGGGGG TGAACACCTT | |
| | | GGTTCTGGTT AAACAGCTGT ACTTTTGATA GCTGTGCCAG | |
| | | GAAGGGTTAG GACCAACTAC AAATTAATGT TGGTTGTCAA | |
| | | ATGTAGTGTG TTTCCCTAAC TTTCTGTTTT TCCTGAGAAA | |
| | | AAAAAATAAA TCTTTTATTC AAATACAGGG AAAAAAAAAA | |
| | | AAAAAAAA | |
| NFKBIZ | NM_001005474.2 | CTCCTCTTGC CACGAGGTCA GACGGCGAGT TCTTAGAGAA | 13 |
| | | AAAGGCTGCT TAGCTGCTGC TTATCATGTA ACCTCAAAAG | |
| | | GAAACTGATC GTCTTTCTCA TGCTGTCACG TACTTGGGTT | |
| | | ATTATCGCTG ATTACAGCTG GAAACAATTG ATTTGCTCTT | |
| | | ACGTATTTGT GTGACTTGAC TCTTCAAACA CAAAGGTTAA | |
| | | CAGGAAGATC TCGAGGGCCC TGGCTGAACT TCACCTTTTG | |
| | | GCTTTCTTGG CCTGATGCTG AACTCTCGAG GTTGAGCCCC | |
| | | ATATGGGGGT TGGCAGGCAG CAGAGAGGCC CCTTTCAAGG | |
| | | TGTTCGGGTA AAGAACTCAG TGAAGGAACT CCTGTTGCAC | |
| | | ATCCGAAGTC ATAAACAGAA GGCTTCTGGC CAAGCTGTGG | |
| | | ATGATTTTAA GACACAAGGT GTGAACATAG AACAGTTCAG | |
| | | AGAATTGAAG AACACAGTAT CATACAGTGG GAAAAGGAAA | |
| | | GGGCCCGATT CGTTGTCTGA TGGACCTGCT TGCAAAAGGC | |
| | | CAGCTCTGTT GCATTCCCAA TTTTTGACAC CACCTCAAAC | |
| | | ACCAACGCCC GGGGAGAGCA TGGAAGATGT TCATCTCAAT | |
| | | GAACCCAAAC AGGAGAGCAG TGCTGATCTG CTTCAGAACA | |
| | | TTATCAACAT TAAGAATGAA TGCAGCCCCG TTTCCCTGAA | |
| | | CACAGTTCAA GTTAGCTGGC TGAACCCCGT GGTGGTCCCT | |
| | | CAGAGCTCCC CCGCAGAGCA GTGTCAGGAC TTCCATGGAG | |
| | | GGCAGGTCTT TTCTCCACCT CAGAAATGCC AACCATTCCA | |
| | | AGTCAGGGGC TCCCAACAAA TGATAGACCA GGCTTCCCTG | |
| | | TACCAGTATT CTCCACAGAA CCAGCATGTA GAGCAGCAGC | |
| | | CACACTACAC CCACAAACCA ACTCTGGAAT ACAGTCCTTT | |
| | | TCCCATACCT CCCCAGTCCC CCGCTTATGA ACCAAACCTC | |
| | | TTTGATGGTC CAGAATCACA GTTTTGCCCA AACCAAAGCT | |
| | | TAGTTTCCCT TCTTGGTGAT CAAAGGGAAT CTGAGAATAT | |
| | | TGCTAATCCC ATGCAGACTT CCTCCAGTGT TCAGCAGCAA | |
| | | AATGATGCTC ACTTGCACAG CTTCAGCATG ATGCCCAGCA | |
| | | GCGCCTGTGA GGCCATGGTG GGGCACGAGA TGGCCTCTGA | |
| | | CTCTTCAAAC ACTTCACTGC CATTCTCAAA CATGGGAAAT | |
| | | CCAATGAACA CCACACAGTT AGGGAAATCA CTTTTTCAGT | |
| | | GGCAGGTGGA GCAGGAAGAA AGCAAATTGG CAAATATTTC | |
| | | CCAAGACCAG TTTCTTTCAA AGGATGCAGA TGGTGACACG | |
| | | TTCCTTCATA TTGCTGTTGC CCAAGGGAGA AGGGCACTTT | |
| | | CCTATGTTCT TGCAAGAAAG ATGAATGCAC TTCACATGCT | |
| | | GGATATTAAA GAGCACAATG GACAGAGTGC CTTTCAGGTG | |
| | | GCAGTGGCTG CCAATCAGCA TCTCATTGTG CAGGATCTGG | |
| | | TGAACATCGG GGCACAGGTG AACACCACAG ACTGCTGGGG | |
| | | AAGAACACCT CTGCATGTGT GTGCTGAGAA GGGCCACTCC | |
| | | CAGGTGCTTC AGGCGATTCA GAAGGGAGCA GTGGGAAGTA | |
| | | ATCAGTTTGT GGATCTTGAG GCAACTAACT ATGATGGCCT | |
| | | GACTCCCCTT CACTGTGCAG TCATAGCCCA CAATGCTGTG | |
| | | GTCCATGAAC TCCAGAGAAA TCAACAGCCT CATTCACCTG | |
| | | AAGTTCAGGA GCTTTTACTG AAGAATAAGA GTCTGGTTGA | |
| | | TACCATTAAG TGCCTAATTC AAATGGGAGC AGCGGTGGAA | |
| | | GCGAAGGATC GCAAAAGTGG CCGCACAGCC CTGCATTTGG | |
| | | CAGCTGAAGA AGCAAATCTG GAACTCATTC GCCTCTTTTT | |
| | | GGAGCTGCCC AGTTGCCTGT CTTTTGTGAA TGCAAAGGCT | |
| | | TACAATGGCA ACACTGCCCT CCATGTTGCT GCCAGCTTGC | |
| | | AGTATCGGTT GACACAATTA GATGCTGTCC GCCTGTTGAT | |
| | | GAGGAAGGGA GCAGACCCAA GTACTCGGAA CTTGGAGAAC | |
| | | GAACAGCCAG TGCATTTGGT TCCCGATGGC CCTGTGGGAG | |
| | | AACAGATCCG ACGTATCCTG AAGGGAAAGT CCATTCAGCA | |
| | | GAGAGCTCCA CCGTATTAGC TCCATTAGCT TGGAGCCTGG | |
| | | CTAGCAACAC TCACTGTCAG TTAGGCAGTC CTGATGTATC | |
| | | TGTACATAGA CCATTTGCCT TATATTGGCA AATGTAAGTT | |
| | | GTTTCTATGA AACAAACATA TTTAGTTCAC TATTATATAG | |
| | | TGGGTTATAT TAAAAGAAAA GAAGAAAAAT ATCTAATTTC | |
| | | TCTTGGCAGA TTTGCATATT TCATACCCAG GTATCTGGGA | |
| | | TCTAGACATC TGAATTTGAT CTCAATGGTA ACATTGCCTT | |
| | | CAATTAACAG TAGCTTTTGA GTAGGAAAGG ACTTTGATTT | |
| | | GTGGCACAAA ACATTATTAA TATAGCTATT GACAGTTTCA | |
| | | AAGCAGGTAA ATTGTAAATG TTTCTTTAAG AAAAAGCATG | |
| | | TGAAAGGAAA AAGGTAAATA CAGCATTGAG GCTTCATTTG | |
| | | GCCTTAGTCC CTGGGAGTTA CTGGCGTTGG ACAGGCTTCA | |
| | | GTCATTGGAC TAGATGAAAG GTGTCCATGG TTAGAATTTG | |
| | | ATCTTTGCAA ACTGTATATA ATTGTTATTT TTGTCCTTAA | |
| | | AAATATTGTA CATACTTGGT TGTTAACATG GTCATATTTG | |
| | | AAATGTATAA GTCCATAAAA TAGAAAAGAA CAAGTGAATT | |
| | | GTTGCTATTT AAAAAAATTT TACAATTCTT ACTAAGGAGT | |
| | | TTTTATTGTG TAATCACTAA GTCTTTGTAG ATAAAGCAGA | |
| | | TGGGGAGTTA CGGAGTTGTT CCTTTACTGG CTGAAAGATA | |
| | | TATTCGAATT GTAAAGATGC TTTTTCTCAT GCATTGAAAT | |
| | | TATACATTAT TTGTAGGGAA TTGCATGCTT TTTTTTTTTT | |
| | | TTCTCCCGAG ACAGGGTCTT GCTCTGGCGC CCAGGCTGGA | |
| | | GTACAGTGGC ATGATCTTGG CTCACTTCAG CCTTGACTTG | |
| | | GGCTCAAGTG ATCCTCCTAC CTGAGCCTTC TGAGTAACTG | |
| | | GAACTACAGG TGTGCACTCC TCGCCTGGCT AATTTTTTAT | |
| | | TTTTTGTACA GGCAGGATCT TGCCACCTTG CCCAGGCTGG | |
| | | TCTTGAACTC CTGAGCTCAT GCCATCTGCC TGCCTTAGTC | |
| | | TCCCAAAATG CTGGGATTAC AGGAGTGAGC CACCATGCCC | |
| | | GGCTGGCAGT TGCATGGAAG AGAACACCTC TTTATGGCTT | |
| | | ACCCTCTAGA ATTTCTAATT TATGTGTTCT GTTGAAATTT | |
| | | TTGTTTTTTT ACCTTTATTG AAACAACAAA AAGTCAGTAT | |
| | | TGAAACATAT CTTCCTGTTT TCTGTTGTCA AATGATGATA | |
| | | ATGTGCCATG ATGTTTTATA TATATCATTC AGAAAAAGTT | |
| | | TTATTTTTTA ATAACATTCT ATTAACATTA TTTTGCTTGC | |
| | | CGCTGGCATG CCTGAGGAAT GTATTTGGCT TTGATTACAC | |
| | | ACTAAGTTTT TGTAATAAAT TTGACTCATT AAAAACCTTT | |
| | | TTTTTTTAAA AAAAAAAAAA AAGAAAATCT CATTAGTGAA | |
| | | CTTATCTTTG CAGCTGAGTA CTTAAATTCT TTTTAAAAAG | |
| | | ATACCCTTTG GATTGATCAC ATTGTTTGAC CCAGTATGTC | |
| | | TTGTAGACAC GTTAGTTATA ATCACCTTGT ATCTCTAAAT | |
| | | ATGGTGTGAT ATGAACCAGT CCATTCACAT TGGAAAAACT | |
| | | GATGGTTTTA AATAAACTAA TTCACTAATA TTAAAAAAAA A | |
| NR4A1 | NM_001202233.1 | TTCCTGGTGT AAGCTTTGGT ATGGATGGTG GCCGTCTCCC | 14 |
| | | TACAGACTGG GAGCTGTTAG AGGGCAGGGA TCCTAGCTGA | |
| | | CACATCTATG TCCTCGCCTT GGTTGGAGGC CTCCACCATG | |
| | | GACAGAGGCC AGGCCCTGCC CCTCCCAGGC AGCCTGGCTC | |
| | | CTTCTGCTGG GCCCTGAAGG CAGACGGGAT AATGTGGTTG | |
| | | GCCAAGGCCT GTTGGTCCAT CCAGAGTGAG ATGCCCTGTA | |
| | | TCCAAGCCCA ATATGGGACA CCAGCACCGA GTCCGGGACC | |
| | | CCGTGACCAC CTGGCAAGCG ACCCCCTGAC CCCTGAGTTC | |
| | | ATCAAGCCCA CCATGGACCT GGCCAGCCCC GAGGCAGCCC | |
| | | CCGCTGCCCC CACTGCCCTG CCCAGCTTCA GCACCTTCAT | |
| | | GGACGGCTAC ACAGGAGAGT TTGACACCTT CCTCTACCAG | |
| | | CTGCCAGGAA CAGTCCAGCC ATGCTCCTCA GCCTCCTCCT | |
| | | CGGCCTCCTC CACATCCTCG TCCTCAGCCA CCTCCCCTGC | |
| | | CTCTGCCTCC TTCAAGTTCG AGGACTTCCA GGTGTACGGC | |
| | | TGCTACCCCG GCCCCCTGAG CGGCCCAGTG GATGAGGCCC | |
| | | TGTCCTCCAG TGGCTCTGAC TACTATGGCA GCCCCTGCTC | |
| | | GGCCCCGTCG CCCTCCACGC CCAGCTTCCA GCCGCCCCAG | |
| | | CTCTCTCCCT GGGATGGCTC CTTCGGCCAC TTCTCGCCCA | |
| | | GCCAGACTTA CGAAGGCCTG CGGGCATGGA CAGAGCAGCT | |
| | | GCCCAAAGCC TCTGGGCCCC CACAGCCTCC AGCCTTCTTT | |
| | | TCCTTCAGTC CTCCCACCGG CCCCAGCCCC AGCCTGGCCC | |
| | | AGAGCCCCCT GAAGTTGTTC CCCTCACAGG CCACCCACCA | |
| | | GCTGGGGGAG GGAGAGAGCT ATTCCATGCC TACGGCCTTC | |
| | | CCAGGTTTGG CACCCACTTC TCCACACCTT GAGGGCTCGG | |
| | | GGATACTGGA TACACCCGTG ACCTCAACCA AGGCCCGGAG | |
| | | CGGGGCCCCA GGTGGAAGTG AAGGCCGCTG TGCTGTGTGT | |
| | | GGGGACAACG CTTCATGCCA GCATTATGGT GTCCGCACAT | |
| | | GTGAGGGCTG CAAGGGCTTC TTCAAGCGCA CAGTGCAGAA | |
| | | AAACGCCAAG TACATCTGCC TGGCTAACAA GGACTGCCCT | |
| | | GTGGACAAGA GGCGGCGAAA CCGCTGCCAG TTCTGCCGCT | |
| | | TCCAGAAGTG CCTGGCGGTG GGCATGGTGA AGGAAGTTGT | |
| | | CCGAACAGAC AGCCTGAAGG GGCGGCGGGG CCGGCTACCT | |
| | | TCAAAACCCA AGCAGCCCCC AGATGCCTCC CCTGCCAATC | |
| | | TCCTCACTTC CCTGGTCCGT GCACACCTGG ACTCAGGGCC | |
| | | CAGCACTGCC AAACTGGACT ACTCCAAGTT CCAGGAGCTG | |
| | | GTGCTGCCCC ACTTTGGGAA GGAAGATGCT GGGGATGTAC | |
| | | AGCAGTTCTA CGACCTGCTC TCCGGTTCTC TGGAGGTCAT | |
| | | CCGCAAGTGG GCGGAGAAGA TCCCTGGCTT TGCTGAGCTG | |
| | | TCACCGGCTG ACCAGGACCT GTTGCTGGAG TCGGCCTTCC | |
| | | TGGAGCTCTT CATCCTCCGC CTGGCGTACA GGTCTAAGCC | |
| | | AGGCGAGGGC AAGCTCATCT TCTGCTCAGG CCTGGTGCTA | |
| | | CACCGGCTGC AGTGTGCCCG TGGCTTCGGG GACTGGATTG | |
| | | ACAGTATCCT GGCCTTCTCA AGGTCCCTGC ACAGCTTGCT | |
| | | TGTCGATGTC CCTGCCTTCG CCTGCCTCTC TGCCCTTGTC | |
| | | CTCATCACCG ACCGGCATGG GCTGCAGGAG CCGCGGCGGG | |
| | | TGGAGGAGCT GCAGAACCGC ATCGCCAGCT GCCTGAAGGA | |
| | | GCACGTGGCA GCTGTGGCGG GCGAGCCCCA GCCAGCCAGC | |
| | | TGCCTGTCAC GTCTGTTGGG CAAACTGCCC GAGCTGCGGA | |
| | | CCCTGTGCAC CCAGGGCCTG CAGCGCATCT TCTACCTCAA | |
| | | GCTGGAGGAC TTGGTGCCCC CTCCACCCAT CATTGACAAG | |
| | | ATCTTCATGG ACACGCTGCC CTTCTGACCC CTGCCTGGGA | |
| | | ACACGTGTGC ACATGCGCAC TCTCATATGC CACCCCATGT | |
| | | GCCTTTAGTC CACGGACCCC CAGAGCACCC CCAAGCCTGG | |
| | | GCTTGAGCTG CAGAATGACT CCACCTTCTC ACCTGCTCCA | |
| | | GGAGGTTTGC AGGGAGCTCA AGCCCTTGGG GAGGGGGATG | |
| | | CCTTCATGGG GGTGACCCCA CGATTTGTCT TATCCCCCCC | |
| | | AGCCTGGCCC CGGCCTTTAT GTTTTTTGTA AGATAAACCG | |
| | | TTTTTAACAC ATAGCGCCGT GCTGTAAATA AGCCCAGTGC | |
| | | TGCTGTAAAT ACAGGAAGAA AGAGCTTGAG GTGGGAGCGG | |
| | | GGCTGGGAGG AAGGGATGGG CCCCGCCTTC CTGGGCAGCC | |
| | | TTTCCAGCCT CCTGCTGGCT CTCTCTTCCT ACCCTCCTTC | |
| | | CACATGTACA TAAACTGTCA CTCTAGGAAG AAGACAAATG | |
| | | ACAGATTCTG ACATTTATAT TTGTGTATTT TCCTGGATTT | |
| | | ATAGTATGTG ACTTTTCTGA TTAATATATT TAATATATTG | |
| | | AATAAAAAAT AGACATGTAG TTGGAACTGA AAAAAAAAAA | |
| | | AAA | |
| PDE4B | NM_001037339.2 | ATCACATACC CTAAAGAACC CTGGGATGAC TAAGGCAGAG | 15 |
| | | AGAGTCTGAG AAAACTCTTT GGTGCTTCTG CCTTTAGTTT | |
| | | TAGGACACAT TTATGCAGAT GAGCTTATAA GAGACCGTTC | |
| | | CCTCCGCCTT CTTCCTCAGA GGAAGTTTCT TGGTAGATCA | |
| | | CCGACACCTC ATCCAGGCGG GGGGTTGGGG GGAAACTTGG | |
| | | CACCAGCCAT CCCAGGCAGA GCACCACTGT GATTTGTTCT | |
| | | CCTGGTGGAG AGAGCTGGAA GGAAGGAGCC AGCGTGCAAA | |
| | | TAATGAAGGA GCACGGGGGC ACCTTCAGTA GCACCGGAAT | |
| | | CAGCGGTGGT AGCGGTGACT CTGCTATGGA CAGCCTGCAG | |
| | | CCGCTCCAGC CTAACTACAT GCCTGTGTGT TTGTTTGCAG | |
| | | AAGAATCTTA TCAAAAATTA GCAATGGAAA CGCTGGAGGA | |
| | | ATTAGACTGG TGTTTAGACC AGCTAGAGAC CATACAGACC | |
| | | TACCGGTCTG TCAGTGAGAT GGCTTCTAAC AAGTTCAAAA | |
| | | GAATGCTGAA CCGGGAGCTG ACACACCTCT CAGAGATGAG | |
| | | CCGATCAGGG AACCAGGTGT CTGAATACAT TTCAAATACT | |
| | | TTCTTAGACA AGCAGAATGA TGTGGAGATC CCATCTCCTA | |
| | | CCCAGAAAGA CAGGGAGAAA AAGAAAAAGC AGCAGCTCAT | |
| | | GACCCAGATA AGTGGAGTGA AGAAATTAAT GCATAGTTCA | |
| | | AGCCTAAACA ATACAAGCAT CTCACGCTTT GGAGTCAACA | |
| | | CTGAAAATGA AGATCACCTG GCCAAGGAGC TGGAAGACCT | |
| | | GAACAAATGG GGTCTTAACA TCTTTAATGT GGCTGGATAT | |
| | | TCTCACAATA GACCCCTAAC ATGCATCATG TATGCTATAT | |
| | | TCCAGGAAAG AGACCTCCTA AAGACATTCA GAATCTCATC | |
| | | TGACACATTT ATAACCTACA TGATGACTTT AGAAGACCAT | |
| | | TACCATTCTG ACGTGGCATA TCACAACAGC CTGCACGCTG | |
| | | CTGATGTAGC CCAGTCGACC CATGTTCTCC TTTCTACACC | |
| | | AGCATTAGAC GCTGTCTTCA CAGATTTGGA GATCCTGGCT | |
| | | GCCATTTTTG CAGCTGCCAT CCATGACGTT GATCATCCTG | |
| | | GAGTCTCCAA TCAGTTTCTC ATCAACACAA ATTCAGAACT | |
| | | TGCTTTGATG TATAATGATG AATCTGTGTT GGAAAATCAT | |
| | | CACCTTGCTG TGGGTTTCAA ACTGCTGCAA GAAGAACACT | |
| | | GTGACATCTT CATGAATCTC ACCAAGAAGC AGCGTCAGAC | |
| | | ACTCAGGAAG ATGGTTATTG ACATGGTGTT AGCAACTGAT | |
| | | ATGTCTAAAC ATATGAGCCT GCTGGCAGAC CTGAAGACAA | |
| | | TGGTAGAAAC GAAGAAAGTT ACAAGTTCAG GCGTTCTTCT | |
| | | CCTAGACAAC TATACCGATC GCATTCAGGT CCTTCGCAAC | |
| | | ATGGTACACT GTGCAGACCT GAGCAACCCC ACCAAGTCCT | |
| | | TGGAATTGTA TCGGCAATGG ACAGACCGCA TCATGGAGGA | |
| | | ATTTTTCCAG CAGGGAGACA AAGAGCGGGA GAGGGGAATG | |
| | | GAAATTAGCC CAATGTGTGA TAAACACACA GCTTCTGTGG | |
| | | AAAAATCCCA GGTTGGTTTC ATCGACTACA TTGTCCATCC | |
| | | ATTGTGGGAG ACATGGGCAG ATTTGGTACA GCCTGATGCT | |
| | | CAGGACATTC TCGATACCTT AGAAGATAAC AGGAACTGGT | |
| | | ATCAGAGCAT GATACCTCAA AGTCCCTCAC CACCACTGGA | |
| | | CGAGCAGAAC AGGGACTGCC AGGGTCTGAT GGAGAAGTTT | |
| | | CAGTTTGAAC TGACTCTCGA TGAGGAAGAT TCTGAAGGAC | |
| | | CTGAGAAGGA GGGAGAGGGA CACAGCTATT TCAGCAGCAC | |
| | | AAAGACGCTT TGTGTGATTG ATCCAGAAAA CAGAGATTCC | |
| | | CTGGGAGAGA CTGACATAGA CATTGCAACA GAAGACAAGT | |
| | | CCCCCGTGGA TACATAATCC CCCTCTCCCT GTGGAGATGA | |
| | | ACATTCTATC CTTGATGAGC ATGCCAGCTA TGTGGTAGGG | |
| | | CCAGCCCACC ATGGGGGCCA AGACCTGCAC AGGACAAGGG | |
| | | CCACCTGGCC TTTCAGTTAC TTGAGTTTGG AGTCAGAAAG | |
| | | CAAGACCAGG AAGCAAATAG CAGCTCAGGA AATCCCACGG | |
| | | TTGACTTGCC TTGATGGCAA GCTTGGTGGA GAGGGCTGAA | |
| | | GCTGTTGCTG GGGGCCGATT CTGATCAAGA CACATGGCTT | |
| | | GAAAATGGAA GACACAAAAC TGAGAGATCA TTCTGCACTA | |
| | | AGTTTCGGGA ACTTATCCCC GACAGTGACT GAACTCACTG | |
| | | ACTAATAACT TCATTTATGA ATCTTCTCAC TTGTCCCTTT | |
| | | GTCTGCCAAC CTGTGTGCCT TTTTTGTAAA ACATTTTCAT | |
| | | GTCTTTAAAA TGCCTGTTGA ATACCTGGAG TTTAGTATCA | |
| | | ACTTCTACAC AGATAAGCTT TCAAAGTTGA CAAACTTTTT | |
| | | TGACTCTTTC TGGAAAAGGG AAAGAAAATA GTCTTCCTTC | |
| | | TTTCTTGGGC AATATCCTTC ACTTTACTAC AGTTACTTTT | |
| | | GCAAACAGAC AGAAAGGATA CACTTCTAAC CACATTTTAC | |
| | | TTCCTTCCCC TGTTGTCCAG TCCAACTCCA CAGTCACTCT | |
| | | TAAAACTTCT CTCTGTTTGC CTGCCTCCAA CAGTACTTTT | |
| | | AACTTTTTGC TGTAAACAGA ATAAAATTGA ACAAATTAGG | |
| | | GGGTAGAAAG GAGCAGTGGT GTCGTTCACC GTGAGAGTCT | |
| | | GCATAGAACT CAGCAGTGTG CCCTGCTGTG TCTTGGACCC | |
| | | TGCCCCCCAC AGGAGTTGTA CAGTCCCTGG CCCTGTTCCC | |
| | | TACCTCCTCT CTTCACCCCG TTAGGCTGTT TTCAATGTAA | |
| | | TGCTGCCGTC CTTCTCTTGC ACTGCCTTCT GCGCTAACAC | |
| | | CTCCATTCCT GTTTATAACC GTGTATTTAT TACTTAATGT | |
| | | ATATAATGTA ATGTTTTGTA AGTTATTAAT TTATATATCT | |
| | | AACATTGCCT GCCAATGGTG GTGTTAAATT TGTGTAGAAA | |
| | | ACTCTGCCTA AGAGTTACGA CTTTTTCTTG TAATGTTTTG | |
| | | TATTGTGTAT TATATAACCC AAACGTCACT TAGTAGAGAC | |
| | | ATATGGCCCC CTTGGCAGAG AGGACAGGGG TGGGCTTTTG | |
| | | TTCAAAGGGT CTGCCCTTTC CCTGCCTGAG TTGCTACTTC | |
| | | TGCACAACCC CTTTATGAAC CAGTTTTGGA AACAATATTC | |
| | | TCACATTAGA TACTAAATGG TTTATACTGA GCTTTTACTT | |
| | | TTGTATAGCT TGATAGGGGC AGGGGGCAAT GGGATGTAGT | |
| | | TTTTACCCAG GTTCTATCCA AATCTATGTG GGCATGAGTT | |
| | | GGGTTATAAC TGGATCCTAC TATCATTGTG GCTTTGGTTC | |
| | | AAAAGGAAAC ACTACATTTG CTCACAGATG ATTCTTCTGA | |
| | | ATGCTCCCGA ACTACTGACT TTGAAGAGGT AGCCTCCTGC | |
| | | CTGCCATTAA GCAGGAATGT CATGTTCCAG TTCATTACAA | |
| | | AAGAAAACAA TAAAACAATG TGAATTTTTA TAATAAAATG | |
| | | TGAACTGATG TAGCAAATTA CGCAAATGTG AAGCCTCTTC | |
| | | TGATAACACT TGTTAGGCCT CTTACTGATG TCAGTTTCAG | |
| | | TTTGTAAAAT ATGTTTCATG CTTTCAGTTC AGCATTGTGA | |
| | | CTCAGTAATT ACAGAAAATG GCACAAATGT GCATGACCAA | |
| | | TGTATGTCTA TGAACACTGC ATTGTTTCAG GTGGACATTT | |
| | | TATCATTTTC AAATGTTTCT CACAATGTAT GTTATAGTAT | |
| | | TATTATTATA TATTGTGTTC AAATGCATTC TAAAGAGACT | |
| | | TTTATATGAG GTGAATAAAG AAAAGCATGA TTAGATTAAA | |
| | | AAAA | |
| P1AS2 | NM_001324046.1 | CGGAGGCGGA GCTCCCGCGC CGTTCACGTG ACGGGCCCGG | 16 |
| | | CTGTAGCGGC GGCGGCCGCG GCGTCTTAAG CGGCGCCCAG | |
| | | TGCAGGATGG TGCTGGAGGC GGCGGCGGCC GTGGTGGCGG | |
| | | CAGCGTCGTT GGCGGCAGCG GGAGTGGGTG CGGCGGCAGC | |
| | | GGCGGCGGCG CCCGCGGGTG GTATAAAATG GCGGATTTCG | |
| | | AAGAGTTGAG GGATCTCCAG TACAATGTTG AATAGAAGCT | |
| | | GTGATGGGGG CATCCTGATC TTGTTCCTGA TTTTAAAGGG | |
| | | AATGCTTTCA AAGTTTCACA AGTGGCTGTA CCATTTTACA | |
| | | TCCCTACCAG CAATGTATGG AGTGATCCAG TTTCTCAGCA | |
| | | TCCTCGCCAG CATTTGGTGT TATCAGTGTT TTTCATTTTA | |
| | | GCCATTCTGA ATATGGTTTC TAGTTTTAGG GTTTCTGAAC | |
| | | TACAAGTATT ACTAGGCTTT GCTGGACGGA ATAAAAGTGG | |
| | | ACGCAAGCAT GACCTCCTGA TGAGGGCGCT GCATTTATTG | |
| | | AAGAGCGGCT GCAGCCCTGC GGTTCAGATT AAAATCCGAG | |
| | | AATTGTATAG ACGCCGATAT CCACGAACTC TTGAAGGACT | |
| | | TTCTGATTTA TCCACAATCA AATCATCGGT TTTCAGTTTG | |
| | | GATGGTGGCT CATCACCTGT AGAACCTGAC TTGGCCGTGG | |
| | | CTGGAATCCA CTCGTTGCCT TCCACTTCAG TTACACCTCA | |
| | | CTCACCATCC TCTCCTGTTG GTTCTGTGCT GCTTCAAGAT | |
| | | ACTAAGCCCA CATTTGAGAT GCAGCAGCCA TCTCCCCCAA | |
| | | TTCCTCCTGT CCATCCTGAT GTGCAGTTAA AAAATCTGCC | |
| | | CTTTTATGAT GTCCTTGATG TTCTCATCAA GCCCACGAGT | |
| | | TTAGTTCAAA GCAGTATTCA GCGATTTCAA GAGAAGTTTT | |
| | | TTATTTTTGC TTTGACACCT CAACAAGTTA GAGAGATATG | |
| | | CATATCCAGG GATTTTTTGC CAGGTGGTAG GAGAGATTAT | |
| | | ACAGTCCAAG TTCAGTTGAG ACTTTGCCTG GCAGAGACAA | |
| | | GTTGCCCTCA AGAAGATAAC TATCCAAATA GTCTATGTAT | |
| | | AAAAGTAAAT GGGAAGCTAT TTCCTTTGCC TGGCTATGCA | |
| | | CCACCGCCTA AAAATGGGAT TGAACAGAAG CGCCCTGGAC | |
| | | GCCCCTTGAA TATTACATCT TTAGTTAGGT TATCTTCAGC | |
| | | TGTGCCAAAC CAAATTTCCA TTTCTTGGGC ATCAGAAATT | |
| | | GGGAAGAATT ACTCTATGTC TGTATATCTT GTACGGCAGC | |
| | | TTACATCAGC CATGTTATTA CAGAGATTAA AAATGAAAGG | |
| | | TATTAGAAAC CCTGATCATT CCAGAGCACT AATTAAAGAA | |
| | | AAACTTACTG CAGATCCTGA TAGTGAAATT GCTACAACTA | |
| | | GCCTTCGGGT ATCCTTGATG TGCCCTTTAG GAAAAATGAG | |
| | | GCTGACAATC CCATGCCGTG CAGTGACTTG TACACATCTG | |
| | | CAGTGTTTTG ATGCTGCCCT CTATCTACAA ATGAATGAGA | |
| | | AAAAGCCCAC CTGGATTTGT CCTGTGTGTG ACAAAAAAGC | |
| | | TGCCTATGAA AGTCTAATAT TAGATGGGCT TTTTATGGAA | |
| | | ATTCTCAATG ACTGTTCTGA TGTAGATGAG ATCAAATTCC | |
| | | AAGAAGATGG TTCTTGGTGT CCAATGAGAC CGAAGAAAGA | |
| | | AGCTATGAAA GTATCCAGCC AACCGTGTAC AAAAATAGAA | |
| | | AGTTCAAGCG TCCTCAGTAA GCCTTGTTCA GTGACTGTAG | |
| | | CCAGTGAGGC AAGCAAGAAG AAAGTAGATG TTATTGATCT | |
| | | TACAATAGAA AGCTCTTCTG ACGAAGAGGA AGACCCTCCT | |
| | | GCCAAAAGGA AATGCATCTT TATGTCAGAA ACACAAAGCA | |
| | | GCCCAACCAA AGGGGTTCTC ATGTATCAGC CATCTTCTGT | |
| | | AAGGGTGCCC AGTGTGACTT CGGTTGATCC TGCTGCTATT | |
| | | CCGCCTTCAT TAACAGACTA CTCAGTACCA TTCCACCATA | |
| | | CGCCAATATC AAGCATGTCA TCAGATTTGC CAGGTTTGGA | |
| | | TTTTCTTTCC CTTATTCCAG TTGATCCCCA GTCTCACCTC | |
| | | ACCCTTAACA GCAAGCAGTA CGTCTGTCAC CACCACCAGC | |
| | | TCCCATGAAA GCAGTACTCA TGTTAGTTCA TCCAGCAGCA | |
| | | GGAGTGAGAC AGGGGTCATA ACCAGCAGTG GAAGTAACAT | |
| | | TCCTGACATC ATCTCATTGG ACTAAAGGAG GACTCACTTG | |
| | | ATTCTGGGAA TCATTCATCA GAACTGCTTT TTCTTGGATC | |
| | | TCTAGTCTGT GGAAACGTTC TTTTTTTTTT TTTTTTAATA | |
| | | ATTTGGTATT TATTGAAAGT CAGATGGATT CTTTTGCTTT | |
| | | CTGAGGGGTG AACACAGAAG ACTGCAACAA GAACCAAATG | |
| | | ACATGCAAGG ATTTTTCTTA ATTATGCTGT ACTCTCAGCA | |
| | | TCAGATACAT TCAACCATAA CTGTATCTTC CTGAGAGATG | |
| | | GATTTCACTT TCATACGTTA ATGGATGGAG TCTACAAATC | |
| | | AGTGAAAAAA GTTTTTGTTA AAATAAAGAG CAATAAAATT | |
| | | ATGTAAATAT TCAAGTAACC TTTTTTCTAA TTAAAAAAAA | |
| | | AATTGTAATC ACTGATTCTA GAATGACAGC AACTTCTGTT | |
| | | TAACCTTACG TGAAGGAAAA AAAATATATG GAAAGAACTT | |
| | | AATGTTTGCT GAATGAGTCC TTTCCATGGA GATTTGCTCT | |
| | | GTTTCATTGA AGTAATGAAG TTTCAGTACG TGGCCAAAGC | |
| | | TTGGATTCCA CTTTTCGTCC TGCATTTCCA CTTTTCTTCG | |
| | | TGATTGCATC AAAGAAAAAT GGCATTCGTG TTACTTGGTT | |
| | | TTTTTCCTGC TTATCATTTA CACTTTGACG TTGTTTGCTA | |
| | | ATGAGTGCTG TGTGAAGTTC TGGGATTTGG CTACTCTTCA | |
| | | TTGGTGATTA TGTTTAAAAG CACTATGCAG ATTCTGCCCT | |
| | | GACATAAAGT GTGTTATTAT GTTTTTAGTA ATTGTACATT | |
| | | TTTCATTCTA GAGTTTTCTA TAAATTTGAG GCTTGCCTTC | |
| | | TCAAAAAAGA AACTATGCAG CCATTGAATG AAATGTCTTT | |
| | | GGGGTACGGT GTGACTGGAA TGTTTGTTAG AAATTTGTTC | |
| | | ACACTATCAA ATATTGATAT CTTGGAGCCA GCAGAAGAGC | |
| | | AGATTTTGGG AGGTGGTAAT AACAAAATTT AATTTCTTCC | |
| | | CAACAACTTA ATTTTCTCAT TTATTTTACA GAATAGTAGT | |
| | | GAAATATTTG ATGAAACTTT GTATTTTGGT AGCACTACAT | |
| | | AGAAAATGTG TTTTAGATTT ATGATGATCA TATTTCTCAC | |
| | | CAATGTAATT TCAGTCTCAG CAGTGATTTT CAAACTTAGG | |
| | | GAAAGGGACA GCATTAGATT TTTTTTTTTT TTCATTTTTT | |
| | | TAAAATGATA TCTTACCTGA AACTACAAAC GACAAAAGAG | |
| | | AATTAGAAAT GTTTGAATTA AAGTGAAGAA GGGTTGGGGG | |
| | | AGATGGGCCT GAACCCACTT CCTGTCTCAA TCCATGCTAC | |
| | | CCCAAACACT CCAGGGAACC TCTGAGGTTT TATTGGGTGC | |
| | | ACTTTGAAAA TTTCTCTTCT ATAGTGTGTT TGTTTGATTT | |
| | | TAAATCACAG AGAAAACTGG GTTTTACTCT TAGAGAAACA | |
| | | TTTTCATCCA GTTTTTTAGT TTGCTTCATT TGACTTCCTA | |
| | | AATCATTTTT GAGTTCACAA GGATTTGGTA CTTTTCTGTT | |
| | | TAGCTTTCTC TCTCTAAGCT TTATCTACCT TAAAAACAAA | |
| | | GTCCTTTTTT TAATGGCCAG TCCAACCAAT TGATTTCTCA | |
| | | AACTGAAGTG CCCAGGTGTG GACTCATCAA TTTCCGTTAG | |
| | | AATAGGGACA TCCTACTTAA GAGTTGGTGC AGCTCCAAGG | |
| | | AGCTGACTTG TCCTTGCTTG GGGTTTTTTT TTTTTTTCTT | |
| | | TCACCTTCTC AAGTTTCCAT GGCCTTTGTG TGTTCTTTTT | |
| | | ATGTTGATTT AAATTCATAT GGTTTTCCAC AAATCCCTTC | |
| | | TTTGGCTACA TTGTCTCCTT ATTCAATGGA TTATCCCTTT | |
| | | GTGGGGGCTG CTTATTTTAA AGATGTTGGG GGGGAAACAA | |
| | | ACCCAAATCT ACGAGCAGTA GTTGCACATA GTTGCCAGTT | |
| | | TTACCTTCTT AGTCATTAGA TTTCCAAACC ATGTTGCAGT | |
| | | TTTTTGGTCC AGATATAGTA TTTCTTTCTA ATAAAGTTTT | |
| | | ATGTTGCTGC TCTAAATACA GATGCAATAT TTATTGACTC | |
| | | TGTAATCAGA TAGAAAAAAC TTAACTTGGT TTGTGTGGTA | |
| | | TGACTTATAA AGAAATGATG TATATTTGTT ATTTTGTTAC | |
| | | CCTTTAGATT GTCAGAGACT CCCCCAATTT AATCAACAAA | |
| | | GTTTTATAAA GTAAATGAAA ATATTAATAG AAATTAGTTT | |
| | | ATTTACTTGG TTCTTATAAC TGATATCTCT GTGCTTTTAT | |
| | | AATTGTGATT TGTTTTTTTG TTTTTTTTTC TATTTTCTGT | |
| | | GAACAGTTTT AATGTTCGGT TTTGGTGTTT TACACTGAAA | |
| | | TTACATATAA ATTTTTAATT TATTTCATAC AGGCAACTTG | |
| | | CATTTTAAAA AATACACTTT GAAGTTTATC ATCTTGAAAT | |
| | | TGGGGCTTAC GTTGTTTATC TGTCTTGAGC ATTAGTACTT | |
| | | TATGACTTTG GCCTTATGGC AACATCATGA TTATTAATCC | |
| | | GTCAGCCTTT AATGTGGTCA CTGTTTCTTA TCCAGACCCT | |
| | | GACTTTCTAG TAGTTTATTT TGCTAGCCCA GATTTCTGCT | |
| | | TAACCAAATA TAAGGGAGTT TCAGAGGGGT GATCTTAGCT | |
| | | GTCACCTAGA TTCTGTCAGC CAACCAGTGA TTGTCTGGAA | |
| | | TATCTTAGGA AATGAACTGT AATTGTCAGC CTCCTAAAAT | |
| | | CAATTTTTTT TTTTTTTTTT TTGAGGCTGA GTCTTGCTCT | |
| | | TGTTGCCCGG GATGGAGTGC AATGGTGCGA TCTTGGCTCA | |
| | | CCGCAACCTC CACCTACAGG GTTCAAGCGA TTCTCCTGCC | |
| | | TCAGCCTCCC GAGTAGCTGG GATTACAGGC ATGTATCACC | |
| | | ACGCCCAGCT AATTTTGTAT TTTTAGTAGA TACGGGGTTT | |
| | | CTCCATTTTG GTCAGGCTGG TCTCGAACTC CCGACCTCAG | |
| | | GTGATCCGCC TGCCTTGGCC TCCCAAAGTG CTGGGATTTA | |
| | | CAGGCGTGAG CCACCGCGCC TGGCCAAAAT AAAATGTTTT | |
| | | TATCTTTCTT CACTTAGAAC TATGAAGGCC TTTTCTTGTT | |
| | | CTGGTAACCC CCCACAGTGT AATTGGTGCT GAAAATAGTT | |
| | | TCGTGCCTGA GGTCTGTTGT CACTCACTCA CTTTCTGACA | |
| | | ATTAGGTGAG CCACTTGAGG ATTTTCTGGC CTGAAGTTAT | |
| | | AAAAACTTGG CATCTGTCCT AAGGTAATTG TAAAGGCAAA | |
| | | ATGAAAGCAT TGAGGTGAAT TTAGGTTATA TGGCATTTAT | |
| | | CTGGTAATGT GTTTCAGAAA AGCTAGAATG AAAATGTGAT | |
| | | GTAAGGGATA GCAAGTGTAG GTTGATTCAT AATTCTTGGT | |
| | | ATAAAACCAA GTTTTCCTTA GTCTTGAGGA ATTTGAAGAA | |
| | | AGGTAAAATG TTGGATTTTA AGCAAAGTAG GATTGACCAG | |
| | | AGACTGTTCT GACTTTGACA TTCAGACCTT TCAGATGTTC | |
| | | TTCCTTCAGA CCAGTCCAAG ACACTCAGCA TTTCCAGCCA | |
| | | AGTTGTCTTT TTTTTCTGCC AATAAAGTAT TCATTAGTTG | |
| | | ACGTTTGTTT GTATTTTAAG TAAAGTCAAT TTTTTTTAAA | |
| | | GCATTAATTT AATTCACGGG TATGCCTTTC ATTTCTCTGG | |
| | | CCTTCAGTTG GTTTAAAAAA AAAAAAAAAA AAAAAAGCAC | |
| | | TTAGCCGGAC CATTCTTTCT TTAGTAAACA TTTTTATTTT | |
| | | CTTAATTTTT CCTGAAAGAT TTTCTTCATT TTCATGGAAT | |
| | | GTCATAATGA CACTACCATT ATTTTCTAGC TCTTTGCATA | |
| | | ATAGAAAAAT GTAGAGCAGT GCACCATTTA AACACGTCTG | |
| | | ACATTGGATT TAGTGGTCCT AGGTTCATGT GATTTGGGAG | |
| | | CCTTAAGTTG TTATTTAGAT TGATTCAGCC TGATTCACCA | |
| | | TCTTTTTTAC TACAGTTCTA CATGAATGTG GTGAAACAGG | |
| | | TAGAAGAAAA TTAAGCATTT CTCTGCTTTT GGAGTCTAAT | |
| | | GTTCCTGCCT AGTGACTGGT TTGTAACACC CCCCTCACCC | |
| | | CTACTTTGTT AGGAACCTGC AATTCCTAAA AGAAATAAAT | |
| | | AAAGCTGTAC CCAATGTCCA TTCTTTAATG CTTGCCTGGT | |
| | | TGAATAATGT TTGTTTTTTC TGTTTATGTC AGTAGTACAT | |
| | | GCTTATTGTG GAAAATTTAG ACAATACAGA AAAGTATAAC | |
| | | AATTAATTGC CTATGATTCT GTCACCAGAA AAAATCACTT | |
| | | GATATTTTTG CATCTTCCTT GCCATTGTCT TTGCTACTTC | |
| | | TGCATATTTT TACTTAGTTT CATCATTCTC AGTGTAATTT | |
| | | ATATCTTGGG GTTTTTCCCC GCAACCTACA GGCATTATTC | |
| | | CATGTCATTA AAACTGTGTC AACTATTATT AATTTTATGG | |
| | | TACTCCATTG TTATGGATGT GCCATGTTTC ATATAACCTT | |
| | | TACTTATTTT GAACAGTTTA GATTATATCT CATAATGCCA | |
| | | CAATGAATGT CTTTGTGTAT AAATTATTTT CTTAGGATAA | |
| | | AATACTGGAG GTGGAATTTC TAGTCAAGGG TATATGAACA | |
| | | CTTTTGTAGT TCTTGATTCA TACCACTTTT CCACATGGGT | |
| | | ATGTTAATTT ATACTGCAGC TAGATGGTAT GGGAAGGGTG | |
| | | GTCTAGCCAT ATTTTCCAAG AACATGCAAC GTTTTATCTT | |
| | | TATGCAGGCA TCTGCACTAA GACCCTCTGC CTTTCTGACT | |
| | | CTATGGAACG GGGTTAACTG ATATGGAGAT CATTGGCAGA | |
| | | GATTTTCCCA AGCTTTCTTA GTTTTAAATA TATCTGACAT | |
| | | GGGCCGGGTA TGGAGGCTCA CGCCTGTAAT CCCAGCACTT | |
| | | TGGGAGGCCA AGGCGGGCAG ATAATCTGAA GTCAGGAGTT | |
| | | GGAGACCAGC CTGGCTAACA TGGAACATGG TGAAACCCCA | |
| | | TCTCTAGCAA AAACATAAAT TAGGTGGGTG TGATGGCAGG | |
| | | CGCCTGTAGT CCCAGCTACT TGGGAGACCG AGGCAGGAGA | |
| | | ATCGCTTGAA CCCAGGAGGC GGAGGATTTG CAGTGATCCG | |
| | | AGATTGTGCC ACTGCACTCC AGCCTGGGTA ACAAAGCAAG | |
| | | ACTCTCTAAA AAAAAAAAAA AAAAAAAAAA AAAATATATA | |
| | | TATATATATA TATATATATA TAAAATCTGA CATGTTTCAG | |
| | | AAACACCTAC GTATGGCTAT CCAAGCTTTT CCCATGATTT | |
| | | TACTGGCACT GTAAAGTTGC ACAAATATTC TTTGTCAGAC | |
| | | TACGTTTTCC ATCTTTAGTT CAAAAGTTTT GTCACTTTTT | |
| | | AAAAAAAGCC TCTTGTACCT AAGGCCTGCC CTTAAGAGGT | |
| | | CAGCTCTCTT TTTCCTCTTC CTGTAGGCCC CATTATGGCC | |
| | | TTTCTGAGCT CCCCAAAGCC AGTAGAATCT ACTGAGCCAA | |
| | | AGGCATTCCA TCCCCAAACT GAAAAGTGGT AAGGCTAAAA | |
| | | GCTATAAAAA CAGGCAAACA TTGCCTGTTT TCACTTTATA | |
| | | TTCCCTTATG TTTCCAATAA GGGTAGCTGC AGGGAAAGAA | |
| | | AAAAACAGAT TGCTACCTTC AGATGTTTGA GGTACAACAT | |
| | | TTTCTTGATT GGCGTAGCAC AATCATTTCC CAAACTGCTT | |
| | | CCTATTTCTG AATGGCTTTT TTAAAAAGCT TTATTTTGTG | |
| | | TTCTCAAAGC TTAATTATTT TAAGTGACGA AGAGTGTTAC | |
| | | CGTGTTGTAG AATAGCATAT GCCTCTTATT TTGGCCTTCA | |
| | | TCAAACTGTT TAATAAATGT TTAGAGTGTT TTGTAAGTAC | |
| | | ACGTGGAATA GAGTGTTACA GAAGTTTTGG TTCCAGACAT | |
| | | CAGGCAGCTT ATAATCAAGG TTCCAAAAAT CAAGAATTCT | |
| | | TAAGCTGGGC ATGGTGGCTC ATGCCTGTAA TCCCAGCACT | |
| | | TTGGGAAGCC AAGGCAGGCA AATCACTTGA GGCCAGGAGT | |
| | | TTGAGACCAG CCTGGCCAAC TTGGTAAAAC CCCATCTCTA | |
| | | CTAAAAATAC AAAAAATTAG GGCATGATGG CTCATGTCTG | |
| | | TAATCCCAGC TACTTGGGAG GCTGAGGGAT GAGAATTGCT | |
| | | TGAACCTGAG AGGCGGAGGT TGCAGTGACT CAGGATCATG | |
| | | ACCCTGTACT CCAGCCTGGG TGACAGAGTG AGACCAAAAA | |
| | | AAAAAAAAAA AAAAAAAAAA AAAAGTAACA GGCATTGTGC | |
| | | AAAGAATTTT CCAAGAATGT CTCATTCTTG CAATGGCTTC | |
| | | TCAGAGGAAA GTGGTGTTAT TCCCCATTTT ACAAATGAAA | |
| | | CAGGTTTAGG TAAGTTTCCC AAGGTCACAG TGAATGAGTA | |
| | | GTGGACTTGG GAGTCTGACC TAGACTTACC CCAAAAGCTC | |
| | | TCTGCGTAAA CTGAAGTGAC GTTAATTAGC TGTGCTATCT | |
| | | AGGAGACTGA TATGGGAATC TAGACTTAAA ATTATGAGAA | |
| | | CTGGTTTCAG ATGATGGCCA GAGTAATAAG GAAGGAGTGG | |
| | | ATGCTGAAGC CATTTCAAAG GAAGACTCAA GATTCAGTGA | |
| | | CTGAATATAG GAGTAAGGGT TGAGTGCATT TTTCATCCAT | |
| | | TTTTCAAGCC TGGGTAACTT GGATGTTAAT GGCACTATCT | |
| | | ACAGAGACAG GAGGCAGTTT AGAGTGAAGA CGAATCATGT | |
| | | ATGGGAGACA GTGATGAATG TTAAGCATGG GATTTCAGAA | |
| | | ATGAAGCATC CATGAGGCTC ATTCCTGGTT TTTCTGCAGT | |
| | | CTTTTAAATA AAAGTACAAA CCTCCCTTGT CATCCATTTT | |
| | | TGCCTTCACT TATCTCCATT CTGTATTATG TTCTAGTCAC | |
| | | GCCCCAGTCA CTGATCCCTT CCTAGGCCAT ACTTGGCTAC | |
| | | ATCTTAGCCT TTTCTTTTGA TTTTTCTCTT CACTGCCTTG | |
| | | CCCATTACAA ATTTGGCTGC CACCTGAGGC CCTGCCATAC | |
| | | CCTACAGCTC ACACCAGCGT GGGCTGTGTT CACATTCTAC | |
| | | ATATCTCAAG ATACAGTGGT TGCTGTCCTT CCCCCCTACT | |
| | | GCATCGTTAC ACGTCTCTAA AAAATCTCTG CCTCTGAAGC | |
| | | TCATGCCAAG AGGTTATACT ATCAGCTACC TCTAGTCTTA | |
| | | TAACTGCCTG ATCATTCCCT TTGATTTGCT GGAAGCTTTG | |
| | | ACTCACAGTG TTCCTTGCCT TCCTTGATTC TGGTCATTAT | |
| | | TATTAATATC TTGAACATTC TATAGATGAT TTATCCAAGT | |
| | | TGGTCTGTGA GGTATTGGTT ACTTTACCTC CAGCATTTTC | |
| | | GTCTACTGAA CCACGCTTTC TGTTTTCCTC TTTGCTACTT | |
| | | GTCCAGACTG TTCTTTCATT ATTGCAGCCT TGAAGAAATC | |
| | | CTCAGTAACC TATCCATCCT CCGACATGAC GTAGCATAGT | |
| | | TCCTTTGCCC ATTCTCATCA GTTATAACAA ATGTTATTGC | |
| | | CCTCTTTTCA GGCCACTTCC TTCTCAGAAG ATGACTTTGC | |
| | | TGCTTAATTC AAACAGAAGG GACAGTCCGA AAGCATGATT | |
| | | TCTTCCCCAC CTATGACCAC CTCCATCCTT TTTTGTTTCC | |
| | | AGTTTCAGAG AATGAGGTAG GCCTTTAAGG CCAACTGACT | |
| | | CTGGTCTCCT CCTGTCATCT TCAGGACCTT CCTCTGTCAG | |
| | | ACTTCCCCTC TCTTATCTCC ATGTCTCTCA CCTCTCTACT | |
| | | GCTGCTTTTC TTGGCTTACA AATGGTCTCA GTCATCCAGT | |
| | | ATTTGAGATT ATATTAGGTG CCAGGCATTG TATCAGGCAC | |
| | | TTGGAACACA AGGCAGGATC CTGCCCCCAC CAAAGTTGTG | |
| | | CTCCAATAGC GTCAACAGAG GTGTGAAATA GGGAGATTAC | |
| | | AATGTTAATG GTAAAAGACA AAGTATAGGG TGCTACAAGA | |
| | | GCATGTAAAT GAGGCACCTC AATGATTTAA TAGAAGTAAT | |
| | | GTGGAGAAAT GTGAGGATTG ATGAAAAGCT TGTCCAAAAA | |
| | | GGGCCAGAAA TGTTTAGAAT TGCTGGAAGT TTGCTAAGGA | |
| | | ATATTGAGAA ATATGCATAG ATAGATCGTG AAGGGCCTTG | |
| | | TAAAACAGTG ATGAGACATT GAAGAGTTTT CATGATCATT | |
| | | AGTAGTAATG AGAATTTGTT GAGTTTGGCA TGATTATCTC | |
| | | CATATTACAA ATGTGGAAAC AGATTCTGAC AGGATAAGCG | |
| | | ACTTACTTGC CCAGGGTCAT GCAGCTTGTA ATTGAGGAGT | |
| | | CAGGTTTCAA ATCCAGTTTT AACTACTTTC TCAGAGGCCT | |
| | | TTCTTGATCA TTCTATCTAA AAAGCAAGTG TTCCCTAACT | |
| | | ATTATCCCAG TACCCTGGTT TATATACTCT TAATACATAA | |
| | | AATAATCCCT GCATATTGTG TGTTTCCCCT ACTAGATTGT | |
| | | AAGCTCCATG AGGGCAGAGG TGATAACTTT TTTCACTTGC | |
| | | ATCCCAGAGC CTAACATGAT ATGGGCTACA TAGAAGGTGC | |
| | | CCAGTAAATA TTGGTGGATG AAGAAATGGT GGATAACCAT | |
| | | TTGCATTTGA CTTATGCTGA CTGGAAATTC AATACCTTTG | |
| | | GAATATATAA TACCTCAGCA GTAAGCATTC ACCAAGAGAG | |
| | | GAAAACAGAA GGAGGTGCAT GTTTAGGGTA GGTGGTACGG | |
| | | GAGATGGATG CTGAGGTGTT GAAAGGCAGC ATACTCACCT | |
| | | TCTGAACTCC AAAACTTACA GGGGCAAGGG CCATTAATGA | |
| | | GGGGTGGGTA GAGCAGTGCA CAGTGGGGAT AATATGGGAG | |
| | | TAGGTGTATT AAACCTGGAA GTGTAAGCAA CTCCTTGTAG | |
| | | ACCCACCCTA CTGTGTATAA AAGGCTGGAG AAATTCTTCA | |
| | | TATGATCTAC TCTAGGTTAT GTACTGAGGA CCACCTTATC | |
| | | TGGGTCAGGT ACACTACACA GAGTGCTACA ATATAATTGT | |
| | | AAACATAATT TATCCTCACT GTGATGGCTA GCCACCTGAC | |
| | | CCAAATCATT ATTAGACTCA ATACTTTGGA ACTGGAACAA | |
| | | AAATCAGTTG ACCTGATGTC TGAAAGACAT GCCCACCTGC | |
| | | TATTTGACAA GCTATAGACA TATCCTACCA ATTGATGGGT | |
| | | AGTAGCACTG AAACTATGAA AGTGGATGAA CTCATCCAGA | |
| | | GGAGTTGATG AGTGAGAAAA GAAGACTGGA AAAGGAACAC | |
| | | AAGGAAATCA AGAAGCAGCA GCTGAGACTG AAGGAAAATA | |
| | | ACGTGGTATT ACAGAGGCCA AAAAATAGAG TATTTCAAGA | |
| | | AGCAAGTGGT TAGTGGAGTT AAAAGCTGCT GATACAACCT | |
| | | AAAAAAGTAA GTATCCATTT TTTAAAAAAC TTTCATTTAA | |
| | | AATACCACCA TATTTTATTG AATCTAAGTA CCTGATTGTA | |
| | | AGACATTAAA GAAAAATTAA ACCTGCTAAT TAAA | |
| PRKAA1 | NM_006251.5 | AGCGCCATGC GCAGACTCAG TTCCTGGAGA AAGATGGCGA | 17 |
| | | CAGCCGAGAA GCAGAAACAC GACGGGCGGG TGAAGATCGG | |
| | | CCACTACATT CTGGGTGACA CGCTGGGGGT CGGCACCTTC | |
| | | GGCAAAGTGA AGGTTGGCAA ACATGAATTG ACTGGGCATA | |
| | | AAGTAGCTGT GAAGATACTC AATCGACAGA AGATTCGGAG | |
| | | CCTTGATGTG GTAGGAAAAA TCCGCAGAGA AATTCAGAAC | |
| | | CTCAAGCTTT TCAGGCATCC TCATATAATT AAACTGTACC | |
| | | AGGTCATCAG TACACCATCT GATATTTTCA TGGTGATGGA | |
| | | ATATGTCTCA GGAGGAGAGC TATTTGATTA TATCTGTAAG | |
| | | AATGGAAGGC TGGATGAAAA AGAAAGTCGG CGTCTGTTCC | |
| | | AACAGATCCT TTCTGGTGTG GATTATTGTC ACAGGCATAT | |
| | | GGTGGTCCAT AGAGATTTGA AACCTGAAAA TGTCCTGCTT | |
| | | GATGCACACA TGAATGCAAA GATAGCTGAT TTTGGTCTTT | |
| | | CAAACATGAT GTCAGATGGT GAATTTTTAA GAACAAGTTG | |
| | | TGGCTCACCC AACTATGCTG CACCAGAAGT AATTTCAGGA | |
| | | AGATTGTATG CAGGCCCAGA GGTAGATATA TGGAGCAGTG | |
| | | GGGTTATTCT CTATGCTTTA TTATGTGGAA CCCTTCCATT | |
| | | TGATGATGAC CATGTGCCAA CTCTTTTTAA GAAGATATGT | |
| | | GATGGGATCT TCTATACCCC TCAATATTTA AATCCTTCTG | |
| | | TGATTAGCCT TTTGAAACAT ATGCTGCAGG TGGATCCCAT | |
| | | GAAGAGGGCC ACAATCAAAG ATATCAGGGA ACATGAATGG | |
| | | TTTAAACAGG ACCTTCCAAA ATATCTCTTT CCTGAGGATC | |
| | | CATCATATAG TTCAACCATG ATTGATGATG AAGCCTTAAA | |
| | | AGAAGTATGT GAAAAGTTTG AGTGCTCAGA AGAGGAAGTT | |
| | | CTCAGCTGTC TTTACAACAG AAATCACCAG GATCCTTTGG | |
| | | CAGTTGCCTA CCATCTCATA ATAGATAACA GGAGAATAAT | |
| | | GAATGAAGCC AAAGATTTCT ATTTGGCGAC AAGCCCACCT | |
| | | GATTCTTTTC TTGATGATCA TCACCTGACT CGGCCCCATC | |
| | | CTGAAAGAGT ACCATTCTTG GTTGCTGAAA CACCAAGGGC | |
| | | ACGCCATACC CTTGATGAAT TAAATCCACA GAAATCCAAA | |
| | | CACCAAGGTG TAAGGAAAGC AAAATGGCAT TTAGGAATTA | |
| | | GAAGTCAAAG TCGACCAAAT GATATTATGG CAGAAGTATG | |
| | | TAGAGCAATC AAACAATTGG ATTATGAATG GAAGGTTGTA | |
| | | AACCCATATT ATTTGCGTGT ACGAAGGAAG AATCCTGTGA | |
| | | CAAGCACTTA CTCCAAAATG AGTCTACAGT TATACCAAGT | |
| | | GGATAGTAGA ACTTATCTAC TGGATTTCCG TAGTATTGAT | |
| | | GATGAAATTA CAGAAGCCAA ATCAGGGACT GCTACTCCAC | |
| | | AGAGATCGGG ATCAGTTAGC AACTATCGAT CTTGCCAAAG | |
| | | GAGTGATTCA GATGCTGAGG CTCAAGGAAA ATCCTCAGAA | |
| | | GTTTCTCTTA CCTCATCTGT GACCTCACTT GACTCTTCTC | |
| | | CTGTTGACCT AACTCCAAGA CCTGGAAGTC ACACAATAGA | |
| | | ATTTTTTGAG ATGTGTGCAA ATCTAATTAA AATTCTTGCA | |
| | | CAATAAACAG AAAACTTTGC TTATTTCTTT TGCAGCAATA | |
| | | AGCATGCATA ATAAGTCACA GCCAAATGCT TCCATTTGTA | |
| | | ATCAAGTTAT ACATAATTAT AACCGAGGGC TGGCGTTTTG | |
| | | GAATGCAATT TGCACAGGGA TTGGAACATG ATTTATAGTT | |
| | | AAAAGCCTAA TATGCAGAAA TGAATTAAGA TCATTTTGTT | |
| | | GTTCATTGTG CAGTATGTAT ATAGCATAAT ATACACAGTG | |
| | | AATTATAGGT CTCAGGCTTA CTTGATTTTT GGCTATTTTA | |
| | | TATTTAGTGT ACACAGGGCT TTGAAATATT AATTTACATA | |
| | | AAGGCCTTCA TATATTATTA CGTGTTATAT ATTACGTGTT | |
| | | ATAAATTTAT TCAATAAATA TTTGCCTAGA ATTCCCAAGA | |
| | | CCTTTATAGG TGATTTTGTT TTCTGGGCTC CTTAACTTCA | |
| | | TAAATAGCTA GTATCTTCCA GCAGTAGTAA CAGTCTGGAT | |
| | | AACTTCTTCC ATATCCCTCC CTCTTTGTTT TTTTGAGACA | |
| | | GTGTCACTTT GTCACCCAGG CTGGAGTGCA ATGGTGTGGT | |
| | | CTCGGCTCAC TGCAACCTCC ACCTCCCGGG TTCAAGTGAT | |
| | | TCTCCCGCCT CAGCTTCCTG AGTAGCTGGA ACTACAGGCG | |
| | | TGTGCCACCA CACCCGGCTA ATTTTTCGTA TTTTTAGTGT | |
| | | AGACGGGGTT TCACTATGTT GCCCAGGCTG GTCTCGAACT | |
| | | CCTGACCGCG TGATCCACCA CCTCAGCTTC CCAAAGTGGT | |
| | | GGGATTACAG GCGTGAGCCA CCGCACCCGG CCTCCATATC | |
| | | CCCCTTTTAA AATTCTGTAG TGTATGGTAA GTCATATCAG | |
| | | ATATCAGACC TAATTTAAAT TTCATTTTAG CTTTACAAGT | |
| | | CCAAAAACAC AGAATTTATA TATTCAGATA CTCTAGCACT | |
| | | AATTTTAGTC TTAAAATATT CCCACGATAT TCTGTACACA | |
| | | AAATGTTCTT TTTGTTACAA GAGCTGAGTT GCATATACTG | |
| | | TAGATAAATC ATATTATTTT TGCCAATTTC ACAAATTCCT | |
| | | CTGGCCCATC ATGTCAGTCA TTATTGAGTA TATGCACACA | |
| | | TTGCTACTTA TTTGATTATG TATCTTTTAA ATTGATTCAG | |
| | | TGCATAGAAA ACTATCTCTT ACAAACTTTA AGTGCTCTGA | |
| | | TATGACTTCC CCCCCAAATT TTATTATGAA CATTTTTAAA | |
| | | AACAGAAAAA TTGAAAAACT GTTTGGTAAG CACATGTATA | |
| | | TCTACCATTT AGATTCAGCA GTTGTTAATG TTTTGTCATT | |
| | | TGTTTTCTCT ATACCTATAT ATGTATAGAT ACAGCTAGTT | |
| | | ATGCATATAT ATGCATATAT GTGTTTGTTT GTGTATGTAT | |
| | | ATATGCTTTT TTCCCCCTGA ACCATTTGGA TGTTACAGAC | |
| | | ATACTTATCA CCGTGAAAAT ACTTCAAGTA TCTCCTACAG | |
| | | ATAATGACAT TCTCCTAAAA ATCCGTAATA CCATTGTAAA | |
| | | AGTAATAATT CCCCAATATC ATCTAATCAA GCCATATTTA | |
| | | AATTTCTGAA GTTAACTCCA AATTTCTTTA TAGCTGATTA | |
| | | TTTCAAACTA GGATCCAATT AAAGTTTACA TATGACACTT | |
| | | GGTTATAACT CTTTAGTTGG ATATAACATT ATTATTATTT | |
| | | TGATAAAATA TGGAACAAAT CAATTCTATT AATAAGTGGT | |
| | | CACATTTGTT TTGGGCTTAA ATTACTTTTT AAAGATACTG | |
| | | GATTTTCCTA AGATTTCTGA TTTACACTGA TATTTTTTTT | |
| | | TGTCATTCTT AATTGCATCA CACAATAGAT GTAAATGAAG | |
| | | ATGTAGTCAC CTCAGATAAA ATTGGTATCG TGTATGATAA | |
| | | TATTGTATCA TTTATATTTG CCTTATGTTA ACTTTAAGAA | |
| | | ATTGATTTTT TTGTATTAAT CATTTTCCCA TTGCAACAGA | |
| | | GCTATATTTT TTCTATTTTA AGAATCATAT TTTAGGATTA | |
| | | TTTTTGGCAA ATACAGTGAG CACTTATGTA ACCAGATGAT | |
| | | AATGAACTCA AATGTCATGA TAGCTTGCAT AAATGGTGAC | |
| | | TCTAGTAGAT TTGACTCAAG CACTTCTAGA ATCATGCACT | |
| | | GAATTCAAAA GAAAAATCTT GCTGCTTTTT GTCCAGGGCT | |
| | | TGTTCTATTC AACTTCTAAT TTGAAAGCTG TACAAAGTAA | |
| | | TAGAAGTTCC ATTTAAATAT GAGTTCAAAA CTGTATTTAC | |
| | | TTTTTATGTG GCCCTCTCTT TAGGGGATTC TAATTTTACT | |
| | | TAGGGTCTCT AAGTGCAGCA TAATGTTCCT GATGTTAACA | |
| | | GAAGACTGTA TTTTTAAAGT TACAAATTTG TATATGGAAT | |
| | | TAAGTAATGG CGCTATATAC GCTGTTGTGG GGAGGGGGGA | |
| | | AGAAAAGGAG GAACCAATTA AATAGGACCT TTTAAAAATT | |
| | | GTTAATTTTG TAAACTTTGC TTCTCTTATA AGTTATTGTG | |
| | | ATTCATTTTA GTTACTGTGT TTTATTTTGA AAATATTTAA | |
| | | ATATTGCACT TCTATAAATA GTATGATAAA TGCACAGACA | |
| | | ATTGCAGTAA ATTCTTTTTT AAGCTAGGAT ATTTGAAATG | |
| | | ACAACCTTTG GTTAAGTGTG TCAAGGTTGC AACAGAATTT | |
| | | TCACAATTTT TTTGTTGTTT GCAAATTGTT ACTAATATTG | |
| | | AAGAGGTAAG GGAGGCAATG CAAATGATTT TTAATCTTTT | |
| | | TTTATTATCT TTTCAGCAGT TTATATTTTT TGTGACTTTA | |
| | | TGCAACCATA TTTTTACTTT GTCTTGACAA CTGAAAGATG | |
| | | TATAAGGTTT TTTGCCAGAA ATGTACTGTA TACATAGTTT | |
| | | TAAGTATAAC AGATTTTACT GATATGTAAA AATTTTGCCA | |
| | | TTAAAATAAA TGATTTCTCA CTGAGAGGAA CTTTTCTACC | |
| | | AGGTTGGGGC ATATGGGAGC TTAATATATC ATATCTAATT | |
| | | TAAAATAATT TCACTGAAAT AAACTCCATT GCTTTTACCT | |
| | | AATTTTTTTC TTGAGATGCT TTTGTAGTTT TTCAGAGTTT | |
| | | TAGATGATTT TATACAAAAT CCTCTGCCTA GCACTGCTCT | |
| | | TTTTGATGTT GTAGTGACAC CATTTACATT GAATTAATGC | |
| | | TTGGTAGCCT GGGGCTAGAT GTGGAACTCC ATGGATCTGT | |
| | | GTTCTGACTG GCACCTTTGG AATGAAAGAA AAGTGTGTGC | |
| | | TGTCCAAATT TTTTCCCCTT AATTCTTTCC CTCATCTTCT | |
| | | CACCCATAAT AGAAATTTTA TTTCCATTGT GAGTTCTGAC | |
| | | AAGAATGAAA TTCCACATAC AACATAACTG TAAATTGTTG | |
| | | GTAGGTAGAA GTTAATATTT GTGGTTCATG TATATTTTGA | |
| | | CCAGAGTATA TTTAAGTATA TAATTTCAGC TTCCTTGATT | |
| | | TAGAAATATG ATATAATAAA GAAAAACTCC ATTTATCATC | |
| | | TGTTA | |
| SCYL2 | NM_001317784.1 | AGGAAGTGAC GTCATGCCCC CGGCCGGCAG GTCTTTTAGT | 18 |
| | | CTTTTTCCCC CTCCCTTACT CTTCGTCCCC GGTCCCTCCC | |
| | | CTCCCCACCC CTTTCCTTCT AGCTCCGACG TTTGCGGCCG | |
| | | CGGGGGCGGC GGAGGATATG GAGTAAAGCC AGAGTCAGTG | |
| | | GCCAGGCACG AAGGCAGAGC AGGAACAGCC AGGAGGCGTT | |
| | | TATTAGGGGG GCGGGGGGAA AGAGCCCCAG CACCGCCCCT | |
| | | CCTGGAAGAA GGAAGAGGTA ACTATAACTA CCCAATATTG | |
| | | CAGCCATGGA GTCCATGCTT AATAAATTGA AGAGTACTGT | |
| | | TACAAAAGTA ACAGCTGATG TCACTAGTGC TGTAATGGGA | |
| | | AATCCTGTCA CTAGAGAATT TGATGTTGGT CGACACATTG | |
| | | CCAGTGGTGG CAATGGGCTA GCTTGGAAGA TTTTTAATGG | |
| | | CACAAAAAAG TCAACAAAGC AGGAAGTGGC AGTTTTTGTC | |
| | | TTTGATAAAA AACTGATTGA CAAGTATCAA AAATTTGAAA | |
| | | AGGATCAAAT CATTGATTCT CTAAAACGAG GAGTCCAACA | |
| | | GTTAACTCGG CTTCGACACC CTCGACTTCT TACTGTCCAG | |
| | | CATCCTTTAG AAGAATCCAG GGATTGCTTG GCATTTTGTA | |
| | | CAGAACCAGT TTTTGCCAGT TTAGCCAATG TTCTTGGTAA | |
| | | CTGGGAAAAT CTACCTTCCC CTATATCTCC AGACATTAAG | |
| | | GATTATAAAC TTTATGATGT AGAAACCAAA TATGGTTTGC | |
| | | TTCAGGTTTC TGAAGGATTG TCATTCTTGC ATAGCAGTGT | |
| | | GAAAATGGTG CATGGAAATA TCACTCCTGA AAATATAATT | |
| | | TTGAATAAAA GTGGAGCCTG GAAAATAATG GGTTTTGATT | |
| | | TTTGTGTATC ATCAACCAAT CCTTCTGAAC AAGAGCCTAA | |
| | | ATTTCCTTGT AAAGAATGGG ACCCAAATTT ACCTTCATTG | |
| | | TGTCTTCCAA ATCCTGAATA TTTGGCTCCT GAATACATAC | |
| | | TTTCTGTGAG CTGTGAAACA GCCAGTGATA TGTATTCTTT | |
| | | AGGAACTGTT ATGTATGCTG TATTTAATAA AGGGAAACCT | |
| | | ATATTTGAAG TCAACAAGCA AGATATTTAC AAGAGTTTCA | |
| | | GTAGGCAGTT GGATCAGTTG AGTCGTTTAG GATCTAGTTC | |
| | | ACTTACAAAT ATACCTGAGG AAGTTCGTGA ACATGTAAAG | |
| | | CTACTGTTAA ATGTAACTCC GACTGTAAGA CCAGATGCAG | |
| | | ATCAAATGAC AAAGATTCCC TTCTTTGATG ATGTTGGTGC | |
| | | AGTAACACTG CAATATTTTG ATACCTTATT CCAAAGAGAT | |
| | | AATCTTCAGA AATCACAGTT TTTCAAAGGA CTGCCAAAGG | |
| | | TTCTACCAAA ACTGCCCAAG CGTGTCATTG TGCAGAGAAT | |
| | | TTTGCCTTGT TTGACTTCAG AATTTGTAAA CCCTGACATG | |
| | | GTACCTTTTG TTTTGCCCAA TGTTCTACTT ATTGCTGAGG | |
| | | AATGCACCAA AGAAGAATAT GTCAAATTAA TTCTTCCTGA | |
| | | ACTTGGCCCT GTGTTTAAGC AGCAGGAGCC AATCCAGATT | |
| | | TTGTTAATTT TCCTACAAAA AATGGATTTG CTACTAACCA | |
| | | AAACCCCTCC TGATGAGATA AAGAACAGTG TTCTACCCAT | |
| | | GGTTTACAGA GCACTAGAAG CTCCTTCCAT TCAGATCCAG | |
| | | GAGCTCTGTC TAAACATCAT TCCAACCTTT GCAAATCTTA | |
| | | TAGACTACCC ATCCATGAAA AACGCTTTGA TACCAAGAAT | |
| | | TAAAAATGCT TGTCTACAAA CATCTTCCCT TGCGGTTCGT | |
| | | GTAAATTCAT TAGTGTGCTT AGGAAAGATT TTGGAATACT | |
| | | TGGATAAGTG GTTTGTACTT GATGATATCC TACCCTTCTT | |
| | | ACAACAAATT CCATCCAAGG AACCTGCGGT CCTCATGGGA | |
| | | ATTTTAGGTA TTTACAAATG TACTTTTACT CATAAGAAGT | |
| | | TGGGAATCAC CAAAGAGCAG CTGGCCGGAA AAGTGTTGCC | |
| | | TCATCTTATT CCCCTGAGTA TTGAAAACAA TCTTAATCTT | |
| | | AATCAGTTCA ATTCTTTCAT TTCCGTCATA AAAGAAATGC | |
| | | TTAATAGATT GGAGTCTGAA CATAAGACTA AACTGGAGCA | |
| | | ACTTCATATA ATGCAAGAAC AGCAGAAATC TTTGGATATA | |
| | | GGAAATCAAA TGAATGTTTC TGAGGAGATG AAAGTTACAA | |
| | | ATATTGGGAA TCAGCAAATT GACAAAGTTT TTAACAACAT | |
| | | TGGAGCAGAC CTTCTGACTG GCAGTGAGTC CGAAAATAAA | |
| | | GAGGACGGGT TACAGAATAA ACATAAAAGA GCATCACTTA | |
| | | CACTTGAAGA AAAACAAAAA TTAGCAAAAG AACAAGAGCA | |
| | | GGCACAGAAG CTGAAAAGCC AGCAGCCTCT TAAACCCCAA | |
| | | GTGCACACAC CTGTTGCTAC TGTTAAACAG ACTAAGGACT | |
| | | TGACAGACAC ACTGATGGAT AATATGTCAT CCTTGACCAG | |
| | | CCTTTCTGTT AGTACCCCTA AATCTTCTGC TTCAAGTACT | |
| | | TTCACTTCTG TTCCTTCCAT GGGCATTGGT ATGATGTTTT | |
| | | CTACACCAAC TGATAATACA AAGAGAAATT TGACAAATGG | |
| | | CCTAAATGCC AATATGGGCT TTCAGACTTC AGGATTCAAC | |
| | | ATGCCCGTTA ATACAAACCA GAACTTCTAC AGTAGTCCAA | |
| | | GCACAGTTGG AGTGACCAAG ATGACTCTGG GAACACCTCC | |
| | | CACTTTGCCA AACTTCAATG CTTTGAGTGT TCCTCCTGCT | |
| | | GGTGCAAAGC AGACCCAACA AAGACCCACA GATATGTCTG | |
| | | CCCTTAATAA TCTCTTTGGC CCTCAGAAAC CCAAAGTTAG | |
| | | CATGAACCAG TTATCACAAC AGAAACCAAA TCAGTGGCTT | |
| | | AATCAGTTTG TACCTCCTCA AGGTTCTCCA ACTATGGGCA | |
| | | GTTCAGTAAT GGGGACACAG ATGAACGTGA TAGGACAATC | |
| | | TGCTTTTGGT ATGCAGGGTA ATCCTTTCTT TAACCCACAG | |
| | | AACTTTGCAC AGCCACCAAC TACTATGACC AATAGCAGTT | |
| | | CAGCTAGCAA TGATTTAAAA GATCTTTTTG GGTGAGGTGT | |
| | | CTTACTTCTA TTTTGAAGGA TTATTTCAGT TTCAATCATG | |
| | | GGTGAGCTGA TTTACATCTT TATATAGTTG GCTTGGAGGA | |
| | | AGTACTTCTA TGGGAAAGTG AACAGTTCTG TGACAGGAAA | |
| | | CATCTCTGTC CATGCCAGCA TAGTAGTTGT ATGGACTTCT | |
| | | AACCAGTTGA GTTTTTTAAA GCATTGAGGA TTTTTTCCTC | |
| | | TTACCAACTC CTCTTCAGGT TTTTAAAGAC CCAGCCCTTC | |
| | | CCAATCTCAA AGAGAAAAAG GAAACTGAGT TATCTTGAAT | |
| | | AACATAACTT TTTAATCAAA TGTTTATTTT GGCTTGTGGA | |
| | | TCTTGGTGTT ATTTAAAAAA TTGAGGTGAT GGTCATTGCA | |
| | | AGCTCATCTA TTAAGTACTA TATGGTACAC AGTCTATGAG | |
| | | TCATTAGTCT TCATTTTAAT ATGTAAAAAA TCTTGATGCT | |
| | | GTATTGATTT GTTTGCATTT AAGATGACAG TGAGAAAATG | |
| | | ATAAGCATAA AGAGAAGTAT CAGGTTATTT GCTTTTTCCA | |
| | | AACTTTTCAG ATGAACTATT GTTTAGTACA GAGACTGAGC | |
| | | AAATACTACA AAATTCAACT TAACCTTCAT TTCATTGGTT | |
| | | TAAATGCGTT ATTAACCATC TTAAGTGCAA ACTAATCATT | |
| | | GTAAATTATA TTTTAGCATG GTCTGCCTCA AATAGTAATG | |
| | | TATTTTTCTG CATTCACTTG GATATATTTA GAATCACTTT | |
| | | TTTCCTCCTG TATCAAGGAA GAGGTATGTG CTGATTTGTT | |
| | | TGGATATTTG ACAAGGCACT CTGATGTGAC TTCCCTGACT | |
| | | ACTACCTTCA TATTTCATTT CAAATTCAAA CTTCTGAGGT | |
| | | TGCAGCATAT ATGAATTGCA TTTTCAAAAG AAGATTTGTA | |
| | | AGAATTAAAC TATATTTATG AGTAAACTTT TGAGGTTTCT | |
| | | GCTGTATTGT TTCAAATGTA ATAAACTTTA CTTCTGTAAA | |
| | | AATTGAGCAG TTGTATCTTC TGACCACCAA CAGATTTTCA | |
| | | GCTTGCCATG ATAGTCTGAC CTCATTAATT ACTGCTACTG | |
| | | AAGTTCAATT TTTTTCTAGG AATTTTAGGA ACCTTTTGTT | |
| | | TAAATATTTT AATTTCTATT AGCCATTTTT AGGAAGGAAA | |
| | | GAATCAATTC TCTTAACAGG AAACATGCTT TATTTTTCAA | |
| | | AACCTTTCTC TGATATTTTT CTTTAATTTG CTGATTATTC | |
| | | AACCACAGAG CCTTATGCTA TAAATGTCAT TTGTATTTTA | |
| | | AAAAATAATA TTCCACTCAT AAAACTTTAA AACCATCTTT | |
| | | CAACGAACTA TATATGTATT ATAGTTGCTG CCATAGAGTT | |
| | | GATGGTTTTT AATTATCTGG AACCAGCAAT CATTTAAAAT | |
| | | AAACCATATT AAGTTTAGTA TGCTGGTATT GTTTATTCAT | |
| | | TTTATATGAA TATTCATTGA AAATATATAC ACAATATATG | |
| | | TAATACACAG CACTTGATTA CAAAATGTAA TTTAATTATA | |
| | | TTATTGCTGG CAGCATTCAG TTAAGAGGGT ACTTTAAAAA | |
| | | ATAGAAGTCA GCTTTCACAT CTGATTTCTG TATGGGCTGT | |
| | | ACTTGGTTAA CTTGATTTTA GAAAAAGGAC TAACAGAATT | |
| | | GCTAAAGAAA TGCATCCAAT AAATGAAAAA CAGTAGGAAG | |
| | | ATCAAATGTT TTTGTCAAAT ATATTCACAA CTTGACCAGA | |
| | | TTAGCTGTCC TGTTTGTAAT GCAATATTAA TATGTCTTTT | |
| | | GGGAAAAAGC CTACATATGG AATAAAATAA GTATTGAAGA | |
| | | ATTTTTCTTT GTAACAATTT AGTAGTCACT GTTTATTGAG | |
| | | AAATTGTTTT TTATTTTGTA AAATAACATG ATGTTAGTGT | |
| | | TGAACTCTTA AACAGAAAGA AAGCTTAATA TAACAGCTTA | |
| | | TAGAACTTGA ACTACTAAAT ATGAAAATAA GTCATTTGAA | |
| | | AAAAATACAG TATGTAAAAT TTGTTCATTC GTTGAGGTAA | |
| | | TGGTGCTATG TTTTTACAAA ATTGTTCCTA CACCTTTTTT | |
| | | CTACTTCAGG TATTTTATTT CAACCATTTC CATCAATTGA | |
| | | ACTGTTACCA TTGCCTTTTT CTGTTGAGAA ATTGCCTCTG | |
| | | AAAAATAGTG CTATTTTTCA GCTTAAGTGT TCTTAAGTGA | |
| | | ATGAAATTTT CAAAGTACTA GATCACCTTA AAATTATTTC | |
| | | ACGTACTGAA GACAATTAAG TCCGTTATGT TTAGAGTAGA | |
| | | AAATGTTTAG GTTAAAGAGC ATCTGTCAAC AGAATCTACA | |
| | | AAAAAGATTC CCTTGCATTT GAATTAGTTC TCTATTCTCC | |
| | | TATTGCTAAA TGTGTGATAT ATAGAGAGGA TGTATAAAAG | |
| | | GAAATGGAAA TAGACTATGT ACTTGTCTGG TTTTTGTTTG | |
| | | TTTTTATTTT GGAATGCTTA TAAGCCTCCT TTACACTGAA | |
| | | TAAGGAAGTA GTTTTTGTTT TCTTTTGACC TGTAAAATAC | |
| | | CTCACATGGT TGTTTTTACA CATGAAAGAA AAAGGTATAT | |
| | | GCGAACATAC CTGATATCAA GAGGAGTATG CACCAAATAA | |
| | | ATTTTAGCTT TGATAAAACT TTCCATAAAA AAAAAA | |
| SMARCD2 | NM_001098426.1 | GTTGGGCGGG GCAGGGAGTT CGTAGCCGCC TCTGGGTAAC | 19 |
| | | TCGACTCGGG CGGCCAAACC TCCGGAGGCC GGGGACGGAA | |
| | | GGCGGGCCCG CAGCAGATCC TGGATCCGGA ATCTCCCGGG | |
| | | CAGGAGCGGA ATCTGTCCCG AACCGGGTCT GTGAGGAACT | |
| | | CGCGAACTTG GATTAGGAAA TCCCGGAGCC CGGATCGACA | |
| | | AATCCCGGAA CCCGGAATTA AGATCGCCAA GTCCCGGATC | |
| | | GCGGAGCACA GAGCACGGAG TGGACTCGAC GCGGAGCCCG | |
| | | GAGTCCGGAT CGCGGCACCG CGGGACGGGA CGGAGCGATG | |
| | | TCGGGCCGAG GCGCGGGCGG GTTCCCGCTG CCCCCGCTAA | |
| | | GCCCTGGCGG CGGCGCCGTG GCTGCGGCCC TGGGAGCGCC | |
| | | GCCTCCCCCC GCGGGACCCG GCATGCTGCC CGGACCGGCG | |
| | | CTCCGGGGAC CGGGTCCGGC AGGAGGCGTG GGGGGCCCCG | |
| | | GGGCCGCCGC CTTCCGCCCC ATGGGCCCCG CGGGCCCCGC | |
| | | GGCGCAGTAC CAGCGACCTG GCATGTCACC AGGGAACCGG | |
| | | ATGCCCATGG CTGGCTTGCA GGTGGGACCC CCTGCTGGCT | |
| | | CCCCATTTGG TGCAGCAGCT CCGCTTCGAC CTGGCATGCC | |
| | | ACCCACCATG ATGGATCCAT TCCGAAAACG CCTGCTTGTG | |
| | | CCCCAGGCGC AGCCTCCCAT GCCTGCCCAG CGCCGGGGGT | |
| | | TAAAGAGGAG GAAGATGGCA GATAAGGTTC TACCTCAGCG | |
| | | AATCCGGGAG CTTGTTCCAG AGTCTCAGGC GTACATGGAT | |
| | | CTCTTGGCTT TTGAGCGGAA GCTGGACCAG ACCATTGCTC | |
| | | GCAAGCGGAT GGAGATCCAG GAGGCCATCA AAAAGCCTCT | |
| | | GACACAAAAG CGAAAGCTTC GGATCTACAT TTCCAATACG | |
| | | TTCAGTCCCA GCAAGGCGGA AGGCGATAGT GCAGGAACTG | |
| | | CAGGGACCCC TGGGGGAACC CCAGCAGGGG ACAAGGTGGC | |
| | | TTCCTGGGAA CTCCGAGTGG AAGGAAAACT GCTGGATGAT | |
| | | CCTAGCAAAC AGAAGAGGAA GTTTTCTTCA TTCTTTAAGA | |
| | | GCCTCGTCAT TGAGCTGGAC AAGGAGCTGT ACGGGCCTGA | |
| | | CAATCACCTG GTGGAGTGGC ACCGGATGCC CACCACCCAG | |
| | | GAGACAGATG GCTTCCAAGT AAAACGGCCT GGAGACCTCA | |
| | | ACGTCAAGTG CACCCTCCTG CTCATGCTGG ATCATCAGCC | |
| | | TCCCCAGTAC AAATTGGACC CCCGATTGGC AAGGCTGCTG | |
| | | GGAGTGCACA CGCAGACGAG GGCCGCCATC ATGCAGGCCC | |
| | | TGTGGCTTTA CATCAAGCAC AACCAGCTGC AGGATGGGCA | |
| | | CGAGCGGGAG TACATCAACT GCAACCGTTA CTTCCGCCAG | |
| | | ATCTTCAGTT GTGGCCGACT CCGTTTCTCC GAGATTCCCA | |
| | | TGAAGCTGGC AGGGTTGCTG CAGCATCCAG ACCCCATTGT | |
| | | CATCAACCAT GTCATTAGTG TCGACCCTAA CGACCAGAAG | |
| | | AAGACAGCCT GTTACGACAT CGATGTGGAG GTGGACGACC | |
| | | CACTGAAGGC CCAAATGAGC AATTTTCTGG CCTCTACCAC | |
| | | CAATCAGCAG GAGATCGCCT CCCTTGATGT CAAGATCCAT | |
| | | GAGACCATTG AGTCCATCAA CCAGCTGAAG ACCCAGAGAG | |
| | | ATTTCATGCT CAGTTTTAGC ACCGACCCCC AGGACTTCAT | |
| | | CCAGGAATGG CTCCGTTCCC AGCGCCGAGA CCTCAAGATC | |
| | | ATCACTGATG TGATTGGAAA TCCTGAGGAG GAGAGACGAG | |
| | | CTGCTTTCTA CCACCAGCCC TGGGCCCAGG AAGCAGTAGG | |
| | | CAGGCACATC TTTGCCAAGG TGCAGCAGCG AAGGCAGGAA | |
| | | CTGGAACAGG TGCTGGGAAT TCGCCTGACC TAACTGCTCA | |
| | | GGGATCTTTC TTCCCAGCCC TGGAGCCTGG AGGGAGACCA | |
| | | CCCTCTGGGT CCTTGCTGGG GCCGCAGACA CGTAGGCTGG | |
| | | GGTGAGGAGT GTCTGCTGTC ACCCTCTACT CTCCAGCTTT | |
| | | AGTCTTATAA ATGTAGTGAT AGGATTCCTT GTTGCTTGGT | |
| | | CCCCAAAGCC TTATACTTTT TGCATTGGCT TTAATTGGGT | |
| | | TCAGCAGATG CCTCCTCTGC CCCCCTGCAG GCAGGCCCAA | |
| | | GTAGGACTGC TGGAGGCTGT GCTTTGACAT TGTAAGACAT | |
| | | TTCCGAACCA AAGGCTGCTG GGTTTGCATG TTTACAGACT | |
| | | CCCCCTGGGG CGAGGGTCAG AGCTGGCTCT GGGGAGCTGG | |
| | | GCTAGGAAGA GGAGGTGCAG CCCAGACTCT TCCTAGCCTT | |
| | | TCTAAACCAA AGTTCTTTGC CATTCCTACA AGCCCAGCCT | |
| | | TGCTGCTGGT TTTTTCCTTT CCTTTGGGTA TTTGCACTAT | |
| | | TTTGGGAGCA AGTTTTCTAT GTGGGAGCCA CTTTTTTTGT | |
| | | ACAGGGGTAA GTTGGGGGTT TTCAGGGAGC CTGTTAGGTG | |
| | | CCTCCTTCTT TTCTTTCCTC AATCTATGCA AGCGGCTCTG | |
| | | GCCGCCATCA TCTCCTGGGA TGCCAGAGGG CTGCCTCTCC | |
| | | AGCGGCTTGG GCCGGGGAGG GGACACTCCA GTTCTCTAGC | |
| | | ATGGCCTGAG GTATGGGGTA TGTGCATGTG GAGGCCAGGG | |
| | | TAAGGTGAAT GGGGAGGCTG GGAGGACTGG TGTTGCCCTT | |
| | | TGGAGCTTGG TGAGGAGGGT GGGCCTAGGG CTTGGCGAGT | |
| | | GCCACATCTG GCAGGTTTGG AAATTTCCAA ATAAATCCTT | |
| | | TTGTCTATTG | |
| SP1 | NM_001251825.1 | GTCCGGGTTC GCTTGCCTCG TCAGCGTCCG CGTTTTTCCC | 20 |
| | | GGCCCCCCCC AACCCCCCCG GACAGGACCC CCTTGAGCTT | |
| | | GTCCCTCAGC TGCCACCATG AGCGACCAAG ATCACTCCAT | |
| | | GGATGAAATG ACAGCTGTGG TGAAAATTGA AAAAGGAGTT | |
| | | GGTGGCAATA ATGGGGGCAA TGGTAATGGT GGTGGTGCCT | |
| | | TTTCACAGGC TCGAAGTAGC AGCACAGGCA GTAGCAGCAG | |
| | | CACTGGAGGA GGAGGGCAGG GTGCCAATGG CTGGCAGATC | |
| | | ATCTCTTCCT CCTCTGGGGC TACCCCTACC TCAAAGGAAC | |
| | | AGAGTGGCAG CAGTACCAAT GGCAGCAATG GCAGTGAGTC | |
| | | TTCCAAGAAT CGCACAGTCT CTGGTGGGCA GTATGTTGTG | |
| | | GCTGCCGCTC CCAACTTACA GAACCAGCAA GTTCTGACAG | |
| | | GACTACCTGG AGTGATGCCT AATATTCAGT ATCAAGTAAT | |
| | | CCCACAGTTC CAGACCGTTG ATGGGCAACA GCTGCAGTTT | |
| | | GCTGCCACTG GGGCCCAAGT GCAGCAGGAT GGTTCTGGTC | |
| | | AAATACAGAT CATACCAGGT GCAAACCAAC AGATTATCAC | |
| | | AAATCGAGGA AGTGGAGGCA ACATCATTGC TGCTATGCCA | |
| | | AACCTACTCC AGCAGGCTGT CCCCCTCCAA GGCCTGGCTA | |
| | | ATAATGTACT CTCAGGACAG ACTCAGTATG TGACCAATGT | |
| | | ACCAGTGGCC CTGAATGGGA ACATCACCTT GCTACCTGTC | |
| | | AACAGCGTTT CTGCAGCTAC CTTGACTCCC AGCTCTCAGG | |
| | | CAGTCACGAT CAGCAGCTCT GGGTCCCAGG AGAGTGGCTC | |
| | | ACAGCCTGTC ACCTCAGGGA CTACCATCAG TTCTGCCAGC | |
| | | TTGGTATCAT CACAAGCCAG TTCCAGCTCC TTTTTCACCA | |
| | | ATGCCAATAG CTACTCAACT ACTACTACCA CCAGCAACAT | |
| | | GGGAATTATG AACTTTACTA CCAGTGGATC ATCAGGGACC | |
| | | AACTCTCAAG GCCAGACACC CCAGAGGGTC AGTGGGCTAC | |
| | | AGGGGTCTGA TGCTCTGAAC ATCCAGCAAA ACCAGACATC | |
| | | TGGAGGCTCA TTGCAAGCAG GCCAGCAAAA AGAAGGAGAG | |
| | | CAAAACCAGC AGACACAGCA GCAACAAATT CTTATCCAGC | |
| | | CTCAGCTAGT TCAAGGGGGA CAGGCCCTCC AGGCCCTCCA | |
| | | AGCAGCACCA TTGTCAGGGC AGACCTTTAC AACTCAAGCC | |
| | | ATCTCCCAGG AAACCCTCCA GAACCTCCAG CTTCAGGCTG | |
| | | TTCCAAACTC TGGTCCCATC ATCATCCGGA CACCAACAGT | |
| | | GGGGCCCAAT GGACAGGTCA GTTGGCAGAC TCTACAGCTG | |
| | | CAGAACCTCC AAGTTCAGAA CCCACAAGCC CAAACAATCA | |
| | | CCTTAGCCCC AATGCAGGGT GTTTCCTTGG GGCAGACCAG | |
| | | CAGCAGCAAC ACCACTCTCA CACCCATTGC CTCAGCTGCT | |
| | | TCCATTCCTG CTGGCACAGT CACTGTGAAT GCTGCTCAAC | |
| | | TCTCCTCCAT GCCAGGCCTC CAGACCATTA ACCTCAGTGC | |
| | | ATTGGGTACT TCAGGAATCC AGGTGCACCC AATTCAAGGC | |
| | | CTGCCGTTGG CTATAGCAAA TGCCCCAGGT GATCATGGAG | |
| | | CTCAGCTTGG TCTCCATGGG GCTGGTGGTG ATGGAATACA | |
| | | TGATGACACA GCAGGTGGAG AGGAAGGAGA AAACAGCCCA | |
| | | GATGCCCAAC CCCAAGCCGG TCGGAGGACC CGGCGGGAAG | |
| | | CATGCACCTG CCCCTACTGT AAAGACAGTG AAGGAAGGGG | |
| | | CTCGGGGGAT CCTGGCAAAA AGAAACAGCA TATTTGCCAC | |
| | | ATCCAAGGCT GTGGGAAAGT GTATGGCAAG ACCTCTCACC | |
| | | TGCGGGCACA CTTGCGCTGG CATACAGGCG AGAGGCCATT | |
| | | TATGTGTACC TGGTCATACT GTGGGAAACG CTTCACACGT | |
| | | TCGGATGAGC TACAGAGGCA CAAACGTACA CACACAGGTG | |
| | | AGAAGAAATT TGCCTGCCCT GAGTGTCCTA AGCGCTTCAT | |
| | | GAGGAGTGAC CACCTGTCAA AACATATCAA GACCCACCAG | |
| | | AATAAGAAGG GAGGCCCAGG TGTAGCTCTG AGTGTGGGCA | |
| | | CTTTGCCCCT GGACAGTGGG GCAGGTTCAG AAGGCAGTGG | |
| | | CACTGCCACT CCTTCAGCCC TTATTACCAC CAATATGGTA | |
| | | GCCATGGAGG CCATCTGTCC AGAGGGCATT GCCCGTCTTG | |
| | | CCAACAGTGG CATCAACGTC ATGCAGGTGG CAGATCTGCA | |
| | | GTCCATTAAT ATCAGTGGCA ATGGCTTCTG AGATCAGGCA | |
| | | CCCGGGGCCA GAGACATATG GGCCATACCC CTTAACCCCG | |
| | | GGATGCAAGG TAGCATGGGT CCAAGAGACA TGGAAGAGAG | |
| | | AGCCATGAAG CATTAAAATG CATGGTGTTG AGAAGAATCA | |
| | | GGAGAGGGAT ACAAGAGAGG AGATGGGGTC CCGGCACCCA | |
| | | TCTGTATCAT CAGTGCCTCT TTGAAGGTGG GAAACATTAG | |
| | | TGAAAATTCT GTTGGTGCCA CGCTTTGATG AGCATTTGTT | |
| | | TGACCCCAGT TTCTTCTTAC ACTTCTTACC CCAGCCTACC | |
| | | CTTCCTGCAT TTCTCTTCTC AGCTCTTCCA TGATGGATTC | |
| | | CCCCCCCTTT CCTAAAGCCA TCATGCCTTG ATAAATATAT | |
| | | ATGATCATTG AAATACTTTT TAACAAAAAA CAGATTCTAT | |
| | | ATTATTATAT ATATATATAT ATATATAAAG ATATATAGAG | |
| | | ATGCATTCAC AGGGGTTGGC TGGGAGGAGG AAGACCATTC | |
| | | TGTGACCAAA ATACCTTGGT CATTTTTTTT ATATTGCCTT | |
| | | ATTTCCCTAT GGCTGAGCCT TGTTGTGACA CATCAAGCTT | |
| | | TTCTGTAGAT GTTGTCTTGG CTTCCCACCA GCTTAAGCGT | |
| | | TCATATGCTC TGCTTTTAGT TCATATATAC ATACATAATG | |
| | | TTTTTCCTTT CTTAATTTTG TCTTTTTGTT TGGGATCAGC | |
| | | TTCTTGCACT CCTTCCCTAA CTCAACTGTT GCCGTCTCAT | |
| | | CTTCTCTCAT CTGATCACTT CATGTTTTGT TTTTGTTACT | |
| | | GCCTGGATGA GGCACTTCTG TCAATTTTTT CAGGACCTTA | |
| | | GTTCCAGCAG CAGAATGGAA AAATCCTTGA AGCCCAGGCT | |
| | | GATGCTTGAA GTAACTGTGG AGGGAGTGTT CAAAATACTA | |
| | | CTGACGCAGG CACCTTCTTG GCGCTGGAGA GTCAAAGGCA | |
| | | TCTCCCTTCA TTAGCTGCTC TGAGCATCAA GAATTAGAAG | |
| | | TCTTTCAGTG GAATTGTACA AGAGTCCCTT TGAAGATAAT | |
| | | AATCTTGGCT CAGTTTGTAT AAACTGTCAA ATTTTCAAAT | |
| | | AATAGGTAGG GGGCTTTCAC TAGGAAAATC ATGTGCTCAG | |
| | | AAGAGGAAAT GACTCGTAGT CAGGTTCAGG AGTTAGTGGA | |
| | | GTATTTGGAC TTTGGTACTG CTGTCTTCCA AGGTAGCTCT | |
| | | AAGTTTTGAT GTGTGGGCTT CTGAGTTTAT ATTCTGAAAG | |
| | | GAAATACACT TCTTTTGAAC ATCCCCACTA GGTTCTTTTC | |
| | | CATTGTCAAT AAGGAGCATC AGCCAGTGAA TCTGTTTCAG | |
| | | GTTTCCATTC TGCAGAACTC CTCCAAAGCA TGTGCTAGTG | |
| | | GCAAGACAGT GGTTCTTATG ATGTTTTCCC TTAACTTTTC | |
| | | CTTGTATGTT CTTGGGTGGT TCCTAAGGGA AAGGGAAGCA | |
| | | CATGATCATG GGAATGATAG CCCAGAACAA AAAGAAATCT | |
| | | TGTCTTACCA CAGTGTTTTA TAGGAGAGAT TGGGAGAAAT | |
| | | CATCCTGTTT TCTCTGTGAC CTGATTTCAG AAGAGACTGA | |
| | | TCCAAAAATT ATAACGGCAG GGAACCTAGT GCATTTGGCA | |
| | | CTGAGATTTA AATGCAACCA GAATTGTCCT CAAGGCCCAG | |
| | | CCATAAAAGC ATTGTCTCTC TCGACCTTCT GGTATCTTGT | |
| | | TAGAGAGCTT TTCACTGTGA GGAAGTGTGG AAAAATAGCT | |
| | | CTGTGTGTGT GTGTGTGTGT GTGTGTGTGT GTGTGTGTAA | |
| | | TCTGTTAGGT TGGGGATAGG TTTTCTGCTA GCCAATATTA | |
| | | AAAGAGACCT GCAATAAAAA AATTACCCTG ATCTGATAGA | |
| | | AAGCAAGTGT TTTTGTATGT GTGGGTGAAT GTGTGTTCAT | |
| | | GCCCGTATAT GTCTACACAC AGATGACAAA TTATATTTGA | |
| | | AATCGTTGGA AAATAAATTC AGATCAAAAT GCCTTTCAGG | |
| | | CCCATTACCT AGAAATCTAT CTTAAAACCT GGGTATGTTC | |
| | | CTAAGGTCAT TTCTTTGCTT ATGCTAAATT AATTACAATT | |
| | | ATGAATGGAG GATATTCTAC TGTACTTTTT TAAAAAGAAA | |
| | | CTATTTTTGT GTTTGAAAGT GAAACCAACA TCCAGATCTA | |
| | | TAGCAGAGTC CTTATTCTTC TCATAAATCT TTTTACTTTG | |
| | | GCTACAAATA GATGATGGTA TGATTCTATT ATATATTTTA | |
| | | TATAAAATCC ATCCAAATTA AGTTTTGGGT AAGTGTGTTG | |
| | | TTTAATCTGA ACTATAGTAA CTTAATACTC TAAACAATAG | |
| | | TTCACTCCAT TTGGTCCTTT CTCCACAGAT GTAATTATGT | |
| | | TTTCAACTCA GGAACTATGG CAAGGAACTT TCCCCAGATC | |
| | | AAATTCTATT AACGCTGAGA TACAAGTCAT CCATGCACAG | |
| | | CCACTATCAT ACCCTTTATT CTCACTGAAA GGCAGAACTC | |
| | | AGAACCTGTT ATTTTATGTC TGTAATCATG TACTTTGGCA | |
| | | TCTTTTGGAG GAAAGGGGCA GGATAACTCA CTGGAATGTA | |
| | | CAGTATTTTG CTAGTGCATT TCAAGGAATG GAATCTTCTC | |
| | | CAGTATGAAA TTACCAGATA TAAAATAATG TAATGATGCT | |
| | | GAGGATATAA GCTTTTAGAA GGTAATTTGA TGGTATTTCT | |
| | | TTCTCGAATG AAAAGCTGCT GGTTTACCCT CAACCCTATT | |
| | | CATTAGCATT ACCATGAGTG AATTTATATC TAATTATTTC | |
| | | CACTTGCCCT GTTCTCTTCA CACCAAGGAA GCTCCAGATC | |
| | | CAGTATCTTG TTTGGCCTCA AAACAGAAGC AGCTTCTTTT | |
| | | GTCTCCCAGC AGTAGTGAGC CACTCAGTCT CTTCCACAGG | |
| | | AAGTTTGGAG CCTACATTCC TTGAGTCAGG AGCTTATTAC | |
| | | AGAAAAACCC CGTTTCCCTG AACTTTTGGC TAACAGAAAT | |
| | | TAATTTAACT GACATGCATA TTGATTCTGA AATTTTTTTC | |
| | | CTAAGTTTTT TTCATTTTTT TGAATGAGTT TTTTAAATTT | |
| | | TTTAGATGAC CAAAACTTGC AGGGCAGGGG ATGCCCAGAA | |
| | | GAGTGGTGAG ATAGTAAAAC ACTTATTCCC TCATCCTTTC | |
| | | AGGTTTTCAG GTTGCCCATT TATATTCATT TACATGTCAT | |
| | | TTGACTGTCT CACTTTTTAC CCAGAACAGT AACAACCCAC | |
| | | ACCGTCTTCC TTCAGGGATT TCCAACTGGC ACTCTGTGGG | |
| | | TGCTACACAG AATGCAATTT AATGGATATT TCTCAGCCTG | |
| | | GTTCAGAATA AATTGATCCT TTGATCCCAG AAAGTATATA | |
| | | CTGAAGTGTG GGATAAAGAT TATGATTAGG GGAGGGTTGG | |
| | | AGACAAAAGC TGTAAATTAC TATGGCTGAT TTATTTCTAC | |
| | | TATATACATA TATATTTTTT GCTTTTGTAT ATCCTATATA | |
| | | GGAAACTAAG CATTGTATTT TTTTTAACAA ATCTAAAAAA | |
| | | GCACTATGAA CTACAGGTGT TTGACTTTCA AAATATATTT | |
| | | TGTATTGTTA ATATCTTCAC ATTGTGTGAA TACTGGAAGC | |
| | | TGCAGATCTT TGCTAGGACG CAATAAATTT ATATACTTTT | |
| | | TGAGGGGTTC TTCTGGGGTG CTAATCAGGC CCCTGTTATG | |
| | | CTTAGGGGGA GCCCTGGTGC TACTTGCTTG AAGTTTTCAG | |
| | | TGTAAGTACC CTGATGCCTT TTGGACCTTG GGATCAGATC | |
| | | AAGAGTTTTG GAGATCAGGT ACCAAGGAAA TAAGGACAGT | |
| | | CTAGCTGCCT CAAGTGAGGG GCCCTTTGCA TAGCTCTCCT | |
| | | TCCCCCTCAC TGAAGCTGGG TAGCCTATTG GGGTTGAGAG | |
| | | GGAAAATGTG AAATCTCAGA ATTTATCTCC CTTAGAAGAG | |
| | | AGCCAGTAAC TTATGTACAA GGATGAAAGA AAGGTCGCAG | |
| | | CAGTAGCTTT GGGGAAAGGG AGGAAGATAT GGCACTTCTC | |
| | | CAACCCCGGA AAACATTGCT TTTGAAAACT GCTGATAAAA | |
| | | TATGAGCCGG TTATTACTTC TGTTTGGGAG ACTGTGCTCT | |
| | | CTGTGGTGCC TCTCTTGGCT CTACTCCACA GATACCAGAC | |
| | | CTCTTCTAAG AGGATGAGCA GACCAGCTTT GAGGTTGACC | |
| | | TGTTTCTCTT TGTCTGCCTT CCCAAAACAC CAGCCCCCAG | |
| | | GAAGACATTA AGCAGCCTTA AGCTTAAATT CCTACTCCCT | |
| | | CTTCCAAATT TGGCTCACTT GCCTTAGATC CAAGGCAGGG | |
| | | AAAGGAAAAG AAGGGGGGTC TCTGGCTTTA TTACTCCCCT | |
| | | AAGTCTTTAC TCTGACTTCC CCAAACCCAG AAAGATTTTC | |
| | | TCCACAGTGT TCATTTGAAA GAGGAGTATT TTGTCCCATT | |
| | | TTCCCCTTCC TCATTATCAA ACAGCCCCAG TCTTCCTTGT | |
| | | CTCTGCTAAG AAAGTAGAGG CATGATGATC TGCCTCTCAA | |
| | | CTGCCCTAAG TCCTAGCTAA GTATCAGGGG AAAAAAAAAA | |
| | | AAAAAAAGCC TAACAAATGG GATTAGACTA GGGCTGCAAG | |
| | | TAGTGAGGAT TTTGTTGATA CCTCTGCTGG GATGTGTGCT | |
| | | TTCCCATATC TTGCCTTCAG GAATTACACT GTGCCTTTTC | |
| | | CCCAGGGATA TGGGCTCTGT CTACCCAGTG CTCCAGTTTC | |
| | | CCGGTAACTG CTCTTGAACA TTGTGGACAA GGGCAGGTCT | |
| | | TCATATTTTT GATCATCCCT TTCTCCCAGT GAAATCCCAT | |
| | | AGCCCTTACC TAGAGTCTAG GGCACAAAGA CTTCGGGGAA | |
| | | GATACACTGA GATTGACCTG AGGAGACATC TACACACACC | |
| | | AGTGGCAGCT GCCCCAGGGC CTGCTTCCCC TTCCTAAGTC | |
| | | TGTCATCCTC TGGAAGGGAT GGGTGGTGCT CCAATCTCTG | |
| | | GTGCCTAAAA ACCCAAGTTT ATTTCTCTCT TAACACTGGC | |
| | | AATAACCAGT CCACACCACT GTTGCCTTTT AAAACCTCTT | |
| | | AATAATCTCA TGCTGTGTTT GTTTTGATTC CAATCCAATT | |
| | | ATCACCAGGG CTGTGTGGGT AAATGCTTTT AAATGCTCTC | |
| | | TCATCTTGTT CTTCCCCCTC ACCCCCCACT CTTAGGTATG | |
| | | TATGATGCTA ATCTTGTCCC TAAGTAAGTT TCTTCCTGCT | |
| | | CCTTTTGTAT CTTCCTTTCT TGTCTTTCCT CCTACCTTTT | |
| | | GTCTCTTGGT GTTTTGGGAC TTTTTTTTTT TTTTTTTTGG | |
| | | CCTTTTGTAC AAAGATTAGT TTCAATGTAG TCTGTAGCCT | |
| | | CCTTTGTAAA CCAATTAAAA AGTTTTTTAA TAAAAAAAAA | |
| | | AAA | |
| SRSF5 | NM_001039465.1 | ATTTTGTGGA GCGCCAGAGC TGCTAAGTGC GTCAGTTGTG | 21 |
| | | GAGTGGCGTA GACGAGTTAA GTCCTGGTCT GCGTGGAGGT | |
| | | CGACGACTCC GTCGCAGACT ACGGACCTGT CTGGGTCTCA | |
| | | GCCGCCAAAG ACCCCGTCCG GTAGGTGAGT GGCTCACTTT | |
| | | GAGGGCAAGC CTTCTCGGAT CGAGGCTTCT TCATGGCCGC | |
| | | TCAGATCGTG AGCGGCCGGG GCTGCTCTCT TTGCGGAGGA | |
| | | TGGCGTCTAA TGAGCGCAGT TGATTCGAGG AAGTACTAGC | |
| | | CGGACATCAT GAGTGGCTGT CGGGTATTCA TCGGGAGACT | |
| | | AAATCCAGCG GCCAGGGAGA AGGACGTGGA AAGATTCTTC | |
| | | AAGGGATATG GACGGATAAG AGATATTGAT CTGAAAAGAG | |
| | | GCTTTGGTTT TGTGGAATTT GAGGATCCAA GGGATGCAGA | |
| | | TGATGCTGTG TATGAGCTTG ATGGAAAAGA ACTCTGTAGT | |
| | | GAAAGGGTTA CTATTGAACA TGCTAGGGCT CGGTCACGAG | |
| | | GTGGAAGAGG TAGAGGACGA TACTCTGACC GTTTTAGTAG | |
| | | TCGCAGACCT CGAAATGATA GACGAAATGC TCCACCTGTA | |
| | | AGAACAGAAA ATCGTCTTAT AGTTGAGAAT TTATCCTCAA | |
| | | GAGTCAGCTG GCAGGATCTC AAAGATTTCA TGAGACAAGC | |
| | | TGGGGAAGTA ACGTTTGCGG ATGCACACCG ACCTAAATTA | |
| | | AATGAAGGGG TGGTTGAGTT TGCCTCTTAT GGTGACTTAA | |
| | | AGAATGCTAT TGAAAAACTT TCTGGAAAGG AAATAAATGG | |
| | | GAGAAAAATA AAATTAATTG AAGGCAGCAA AAGGCACAGT | |
| | | AGGTCAAGAA GCAGGTCTCG ATCCCGGACC AGAAGTTCCT | |
| | | CTAGGTCTCG TAGCCGATCC CGTTCCCGTA GTCGCAAATC | |
| | | TTACAGCCGG TCAAGAAGCA GGAGCAGGAG CCGGAGCCGG | |
| | | AGCAAGTCCC GTTCTGTTAG TAGGTCTCCC GTGCCTGAGA | |
| | | AGAGCCAGAA ACGTGGTTCT TCAAGTAGAT CTAAGTCTCC | |
| | | AGCATCTGTG GATCGCCAGA GGTCCCGGTC CCGATCAAGG | |
| | | TCCAGATCAG TTGACAGTGG CAATTAAACT GTAAATAACT | |
| | | TGCCCTGGGG GCCTTTTTTT AAAAAACAAA AACCACAAAA | |
| | | ATTCCCAAAC CATACTTGCT AAAAATTCTG GTAAGTATGT | |
| | | GCTTTTCTGT GGGGGTGGGA TTTGGAAGGG GGGTTGGGTT | |
| | | GGGCTGGATA TCTTTGTAGA TGTGGACCAC CAAGGGGTTG | |
| | | TTGAAAACTA ATTGTATTAA ATGTCTTTTG ATAAGCCTTC | |
| | | TGCTCACATT TTTGTGAATG TCTGAAGTAT ATAGTTTGTG | |
| | | TATATTGACA GAGCTCTTTT ATAACTAAAG CAAATTTAAT | |
| | | TTTTTTGTAC TAGAAAAAAA TTTGAACATT TTAGTTCTTG | |
| | | GTTATAAAAA TGTTAATTCA GAATTAGTTT AATGCCTTAA | |
| | | TTAAACTAAT TAATAGCTTT GGACACTTAA AAGAGCTCTA | |
| | | AATTTGCTTG TACATAAAGG CTTAATTTGT TTTCCTTGTT | |
| | | AGGGTCAAGG GTGTCCTCCA CTCTTTAACA GCTGCTGGAC | |
| | | AGACACATTA GAGCAGCTGT TTGTTATTGA TAATAAAATA | |
| | | TTATAAAACT A | |
| TAGAP | NM_001278733.1 | ATACTATTGA CCACATTATG CGGCCAACGA TACCCATTTT | 22 |
| | | CTTACCTGGA TCTTTGCTTC TTAACACTGT AGTCTAATTT | |
| | | TTCTCGTTGC CTTGTTTTCT TGGCTGTAGA AGTTAAGAAG | |
| | | AGAAAGAAGG TGCTGTCCTG GCCCTTTCTC ATGAGAAGGC | |
| | | TCTCCCCTGC ATCAGATTTT TCTGGGGCTT TGGAGACAGA | |
| | | CTTGAAAGCA TCGCTATTTG ATCAGCCCTT GTCAATTATC | |
| | | TGCGGTGACA GTGACACACT CCCCAGACCC ATCCAGGACA | |
| | | TTCTCACTAT TCTATGCCTT AAAGGCCCTT CAACGGAAGG | |
| | | GATATTCAGG AGAGCAGCCA ACGAGAAAGC CCGTAAGGAG | |
| | | CTGAAGGAGG AGCTCAACTC TGGGGATGCG GTGGATCTGG | |
| | | AGAGGCTCCC CGTGCACCTC CTCGCTGTGG TCTTTAAGGA | |
| | | CTTCCTCAGA AGTATCCCCC GGAAGCTACT TTCAAGCGAC | |
| | | CTCTTTGAGG AGTGGATGGG TGCTCTGGAG ATGCAGGACG | |
| | | AGGAGGACAG AATCGAGGCC CTGAAACAGG TTGCAGATAA | |
| | | GCTCCCCCGG CCCAACCTCC TGCTACTCAA GCACTTGGTC | |
| | | TATGTGCTGC ACCTCATCAG CAAGAACTCT GAGGTGAACA | |
| | | GGATGGACTC CAGCAATCTG GCCATCTGCA TTGGACCCAA | |
| | | CATGCTCACC CTGGAGAATG ACCAGAGCCT GTCATTTGAA | |
| | | GCCCAGAAGG ACCTGAACAA CAAGGTGAAG ACACTGGTGG | |
| | | AATTCCTCAT TGATAACTGC TTTGAAATAT TTGGGGAGAA | |
| | | CATTCCAGTG CATTCCAGTA TCACTTCTGA TGACTCCCTG | |
| | | GAGCACACTG ACAGTTCAGA TGTGTCGACC CTGCAGAATG | |
| | | ACTCAGCCTA CGACAGCAAC GACCCTGATG TGGAATCCAA | |
| | | CAGCAGCAGT GGCATCAGCT CTCCCAGCAG GCAGCCCCAG | |
| | | GTGCCCATGG CCACAGCTGC TGGCTTGGAT AGCGCGGGCC | |
| | | CACAGGATGC CCGAGAGGTC AGCCCAGAGC CCATTGTGAG | |
| | | CACCGTGGCC AGGCTGAAAA GCTCCCTCGC ACAGCCCGAT | |
| | | AGGAGATACT CAGAGCCCAG CATGCCATCC TCCCAGGAGT | |
| | | GCCTCGAGAG CCGGGTGACA AACCAAACAC TAACAAAGAG | |
| | | TGAAGGGGAC TTCCCCGTGC CCCGGGTAGG CTCTCGTTTG | |
| | | GAAAGTGAGG AGGCTGAAGA CCCATTTCCA GAGGAGGTCT | |
| | | TCCCTGCAGT GCAAGGCAAA ACCAAGAGGC CGGTGGACCT | |
| | | GAAGATCAAG AACTTGGCCC CGGGTTCGGT GCTCCCGCGG | |
| | | GCACTGGTTC TCAAAGCCTT CTCCAGCAGC TCGCTGGACG | |
| | | CGTCCTCTGA CAGCTCGCCC GTGGCTTCTC CTTCCAGTCC | |
| | | CAAAAGAAAT TTCTTCAGCA GACATCAGTC TTTCACCACA | |
| | | AAGACAGAGA AAGGCAAGCC CAGCCGAGAA ATTAAAAAGC | |
| | | ACTCCATGTC TTTCACCTTT GCCCCTCACA AAAAAGTGCT | |
| | | GACCAAAAAC CTCAGCGCGG GCTCTGGGAA ATCGCAAGAC | |
| | | TTTACCAGGG ACCACGTCCC GAGGGGTGTC AGAAAGGAAA | |
| | | GCCAGCTTGC CGGCCGAATC GTGCAGGAAA ATGGGTGTGA | |
| | | AACCCACAAC CAAACAGCCC GCGGCTTCTG CCTGAGACCC | |
| | | CACGCCCTCT CGGTGGATGA TGTGTTCCAG GGAGCTGACT | |
| | | GGGAGAGGCC TGGAAGCCCA CCCTCTTATG AAGAGGCCAT | |
| | | GCAGGGCCCG GCAGCCAGAC TAGTGGCCTC CGAGAGCCAG | |
| | | ACCGTGGGGA GCATGACGGT GGGGAGCATG AGGGCGAGGA | |
| | | TGCTGGAGGC GCACTGCCTC CTACCCCCTC TTCCACCTGC | |
| | | TCACCACGTA GAGGACTCAA GACACAGGGG CAGCAAAGAG | |
| | | CCACTCCCTG GCCACGGACT CTCTCCCCTG CCTGAGCGAT | |
| | | GGAAACAGAG CAGAACTGTC CATGCTTCTG GGGACTCTCT | |
| | | GGGGCACGTG TCTGGCCCAG GGAGACCTGA GCTCCTCCCG | |
| | | CTGAGGACCG TCTCCGAGTC CGTGCAGAGG AATAAGCGGG | |
| | | ACTGTCTCGT GCGACGATGT AGCCAGCCGG TCTTTGAGGC | |
| | | TGACCAATTC CAATATGCCA AAGAATCGTA TATTTAGGAG | |
| | | GGAGGCCATA CGCCATGCCA TAGCTTGTGC TATCTGTAAA | |
| | | TATGAGACTT GTAAAGAACT GCCTGTAGAT TGTTTTTAAA | |
| | | AGGTCTTGAA TAAGCTCCTT GAGAAAGTTG TGGAAAGCCC | |
| | | TCCTCAGTGA GGATAGCTAC ACCATGGCCA TGGCGCATCA | |
| | | GATAGTCTCT GTGTACCTGG ATTTGTGCAA TATGTAAAAA | |
| | | TGTATCAAAT GTATTATAGA TAAGGTGTTA GGTGCAAAGG | |
| | | ATGTCTAATA ATCCCTGCAC ACGTTTTGAA CTTGCAGTGA | |
| | | AGTACACTGC TGTTCCTTGC TTCCTGGGGC ACTTTTCTCT | |
| | | TGGTTAGTGT TTAAAAATTA TCTTCGCTTT TTTAATGTGG | |
| | | CCTCAAATGT CATGCCAATT TTCACATCTT CCACAAACTC | |
| | | CATTTAGGGA GAAATGTTTA AATCTCTGGT ATAAGTTTAC | |
| | | TCCATACCAG AGTAAACTAT ATATTACTCT ATATAAGCAG | |
| | | TCTTGCAATA ACTAATCACC ACCATAGAAG AAAGAAACAG | |
| | | ACTGCAAGGA ACAGAGTTGA GTGTCTGGAG TCATCAAAGG | |
| | | CATTAAAAAC TCCAGTAAAA GCTGGGGCCG TAGCAAAAAT | |
| | | CATGAAAAAC ACTTCAACGT GTCCTTTCAA TCATCCAATT | |
| | | AAATGTGGGT AGATTAATGA AAATGTATTA CATCAATATT | |
| | | AACTCATCTA TAGCACTTTG AGTATCTTTG TAGTTCATGA | |
| | | TATCCTATCC TATAATGTGG AGGTAAATGA TTTTATATGC | |
| | | ATTGGGGGTC ATATATAAAA CTTCAATGTA ATTTCACTAC | |
| | | AATAAATTGC CTTCCTTATT TGAAAGTAAA AATGTTCTTT | |
| | | TTTTATTGAC ATGTTAACTC CTTTCCCTCA ATTAATAGAA | |
| | | ATCCACTAAG AAAATCATTC ACATATTGGT TCACTCAACA | |
| | | AGCATTTATT AAATATATAT TCACTATTCT AGACTAATAG | |
| | | CAAGACCGGG GATCTTGTTT AGGAAGAAGC AGTCCCTGTT | |
| | | CTCCAGAATT TGCAAAACCA TTAAAAAAGC ACCTACTTTA | |
| | | AGCCATTTTT TTTCAGCAAG GAGTCATTCT GCCAGAAAAA | |
| | | TGTAGTACAC AAATACAGGA TAATATAACA AATGTAAAAT | |
| | | TTCTCATTTC TAGTGAATTA AACTTTCCAG TAATTTTACA | |
| | | TCTCACCTCA TTCTTATGAT GCTCAGTTTG CTTAATTATT | |
| | | GGCAAACATA ATGGTAAAAT GTTTGTACTG TATTAGAGCT | |
| | | TTACTGTTCG ATTATTAAGA TATTTATCCA GTATCTTAGA | |
| | | GATGCTGGAC TTCAATTTTC CTTATTTTAT CTTTTTAAAA | |
| | | TAAAATATCA TGCATGCTTC TGATTTGTTA TAGAAA | |
| TANK | NM_001199135.1 | ACTCGGCCAG GCGCCGGCGA CCTGAGGGGA GAGGGAACGC | 23 |
| | | AGCTGAAAGC GTGAACTGTA CCTGTCATTT ACTCCATCCT | |
| | | TTATAGTGAT GCTACAGGAC GAAGAGGAAT GGATAAAAAC | |
| | | ATTGGCGAGC AACTCAATAA AGCGTATGAA GCCTTCCGGC | |
| | | AGGCATGCAT GGATAGAGAT TCTGCAGTAA AAGAATTACA | |
| | | GCAAAAGACT GAGAACTATG AGCAGAGAAT ACGTGAACAA | |
| | | CAGGAACAGC TGTCACTTCA ACAGACTATT ATTGACAAGC | |
| | | TAAAATCTCA GTTACTTCTT GTGAATTCCA CTCAAGATAA | |
| | | CAATTATGGC TGTGTTCCTC TGCTTGAAGA CAGTGAAACA | |
| | | AGAAAGAATA ATTTGACTCT TGATCAGCCA CAAGATAAAG | |
| | | TGATTTCAGG AATAGCAAGA GAAAAACTAC CAAAGGTAAG | |
| | | AAGACAAGAG GTTTCTTCTC CTAGAAAAGA AACTTCAGCA | |
| | | AGGAGTCTTG GCAGTCCTTT GCTCCATGAA AGGGGTAATA | |
| | | TAGAGAAGAC TTTCTGGGAT CTGAAAGAAG AATTTCATAA | |
| | | AATATGCATG CTAGCAAAAG CACAGAAAGA CCACTTAAGC | |
| | | AAACTTAATA TACCAGACAC TGCAACTGAA ACACAGTGCT | |
| | | CTGTGCCTAT ACAGTGTACG GATAAAACAG ATAAACAAGA | |
| | | AGCGCTGTTT AAGCCTCAGG CTAAAGATGA TATAAATAGA | |
| | | GGTGCACCAT CCATCACATC TGTCACACCA AGAGGACTGT | |
| | | GCAGAGATGA GGAAGACACC TCTTTTGAAT CACTTTCTAA | |
| | | ATTCAATGTC AAGTTTCCAC CTATGGACAA TGACTCAACT | |
| | | TTCTTACATA GCACTCCAGA GAGACCCGGC ATCCTTAGTC | |
| | | CTGCCACGTC TGAGGCAGTG TGCCAAGAGA AATTTAATAT | |
| | | GGAGTTCAGA GACAACCCAG GGAACTTTGT TAAAACAGAA | |
| | | GAAACTTTAT TTGAAATTCA GGGAATTGAC CCCATAGCTT | |
| | | CAGCTATACA AAACCTTAAA ACAACTGACA AAACAAAGCC | |
| | | CTCAAATCTC GTAAACACTT GTATCAGGAC AACTCTGGAT | |
| | | AGAGCTGCGT GTTTGCCACC TGGAGACCAT AATGCATTAT | |
| | | ATGTAAATAG CTTCCCACTT CTGGACCCAT CTGATGCACC | |
| | | TTTTCCCTCA CTCGATTCCC CGGGAAAAGC AATCCGAGGA | |
| | | CCACAGCAGC CCATTTGGAA GCCCTTTCCT AATCAAGACA | |
| | | GTGACTCGGT GGTACTAAGT GGCACAGACT CAGAACTGCA | |
| | | TATACCTCGA GTATGTGAAT TCTGTCAAGC AGTTTTCCCA | |
| | | CCATCCATTA CATCCAGGGG GGATTTCCTT CGGCATCTTA | |
| | | ATTCACACTT CAATGGAGAG ACTTAAGACA CATTTGAAAA | |
| | | CAGACATATC AAGTTCTATG TGATGATTTT GGGTTTTTAA | |
| | | TACTATAAAT ACTTGATTGT AAACTAAATT CAAGATCATT | |
| | | TATAGGAAAA TCTAGTTTCA CAGCTATTTG AATTTTTTTC | |
| | | TGGATTTACT ATATAACTCT TATTTTTTAA AAGATCATTC | |
| | | TGTTCTTTCA AGGAGAAATA AGCCTAAAAG AAGAAAAACA | |
| | | AAAAAAATTC TGTATAAAAC TGTAATCCTT TGTATTCATG | |
| | | TTTACAGTGC TATTACTATA ATTCAAAATT ATGTATGTGA | |
| | | CTTAGAGTTA TATAATCATA ATTTATGTTT ATTTCAAATA | |
| | | TCTAAGTTTA TTGCTTGGAT TTCTAGTGAG AGCTGTTGAA | |
| | | TTTGGTGATG TCAAATGTTT CTAGGGTTTT TTTAGTTTGT | |
| | | TTTTATTGAA AAATTTAATT ATTTATGCTA TAGGTGATAT | |
| | | TCTCTTTGAA TAAACCTATA ATAGAAAATA GCAGACAACA | |
| | | TAAACATCTT TGTAAATATC AAACCTAATA CATTTCTTGT | |
| | | CCAGTGATAA AACAACTGGT AGAATTATTT AAACACTTTA | |
| | | GATTTTTAAA TAATATACAT GGCTTTAATT TTTACTGTGT | |
| | | GTATAGCTAC ATGATGAAAT TAATTAAATA TTAAGAGGTA | |
| | | AAAATAAAAA AAAAAAAAAA | |
| TLE4 | NM_001282748.1 | GGAGAGCTCG CCAGAGCGCT CGCATGGCGG GCCGGTGATT | 24 |
| | | GTAGTCAATC TGGCCGTATT CTCAGGCAGG GTCGCCCGGG | |
| | | GCGGACTACA TCTCCCGGGA TGCTGCGCGG CCGCCCCGCG | |
| | | GAAGATTGTG AATATGTATC AGAATGTTAA TGATTAGCTG | |
| | | CTGCTAAATT TGGTCAAAGA AGTCACCTAC ACAGAGCGTG | |
| | | TTGTTAGAGC TGTGCTGAGC GGGTGTTTGG GTTGTTGGCT | |
| | | GCTTTCTTCC CCCTTTCTCA CACACTTGTA TATTATTTTG | |
| | | AGGTGGTGTT CGCAGAGTTT GAAAGGAGAG AGAATTAAAA | |
| | | AAAAAAGCCG CAAGCGTTTC ACTCTTTTAT TTTTATAATC | |
| | | CCCTTCAATT TGGGGTTAAA AAAAAGACAA GAAAACAGGA | |
| | | AGGAAGAGAA ATAAGGAAAT GAGATGTGGT AAAAGAAGCT | |
| | | AAAAGGTGCC TTTTAAAAGA TCGTTGCTGT GAAGTGAAAA | |
| | | AAATCTCCAG AGAAACCAAA AAGCACCGCC GAGACCTCTT | |
| | | CCGAACCAAA GGAGTTTGTG TTTGCTTTTA GGGAAGAAGA | |
| | | AAGATCATTC ATTCGGAGGA ATAACAACCA ATTAAAAGAC | |
| | | AAATAAAAAA AGTTTGGAGT GGGACGCAGA GCGAGCGAGA | |
| | | GGAGCTGCCG GCGGGCGGTG GGGCGCGGAG CCCGCACTTT | |
| | | CCCGGCCGGG TGAGCGGCGG CCGCGGCGCC GGGCTCGGCG | |
| | | GGTGCGCCTC GGCGGAGCGA ACGTCGGAGC GTTGCCTTGG | |
| | | GAGACGCGCG CCGGACAATG CCCGCGGCGG GCCAGTGACG | |
| | | CCCGCGGGGA ATGCGGAGCG GCCCGGCAGC CGGCACCCAG | |
| | | CCGCCGCCGC GCGTTCCTGC CGCCCGTGTC ACGCGAGACC | |
| | | CGGCGGGGGC CGGGACCGCC CGAGCCGCCC CTCAGACCGA | |
| | | GCCGGCCGCC TCCGCTGCCG CGGCCGCCTC CTCTTCGGGG | |
| | | TCATTAAAGC CAATGAGCCG CGCGCCTCTG CCGAGCGCAG | |
| | | CCAACTAAAT CGGCTTGGAT GATTCGCGAC CTGAGCAAGA | |
| | | TGTACCCGCA GACCAGACAC CCGGCACCGC ATCAGCCTGC | |
| | | TCAACCCTTT AAATTTACAA TTTCCGAATC CTGTGATCGG | |
| | | ATTAAGGAAG AGTTTCAGTT TTTACAGGCT CAATACCACA | |
| | | GTCTGAAGCT GGAATGTGAG AAACTCGCCA GTGAGAAGAC | |
| | | AGAGATGCAG CGGCATTATG TCATGTATTA TGAAATGTCC | |
| | | TATGGGTTGA ATATAGAAAT GCACAAGCAG GCAGAGATTG | |
| | | TCAAGAGGCT GAATGCTATC TGTGCACAAG TCATTCCTTT | |
| | | CCTGTCCCAA GAGCACCAGC AACAAGTGGT GCAGGCTGTG | |
| | | GAACGGGCCA AGCAGGTGAC CATGGCAGAA CTGAACGCCA | |
| | | TCATTGGGCA ACAACTCCAG GCCCAGCATT TATCACATGG | |
| | | ACATGGTCTC CCCGTACCTC TGACTCCACA CCCTTCAGGG | |
| | | CTCCAGCCCC CTGCCATTCC ACCCATCGGT AGCAGTGCCG | |
| | | GGCTTCTGGC CCTCTCCAGT GCTCTAGGAG GTCAGTCCCA | |
| | | TCTTCCAATT AAAGATGAGA AGAAGCACCA TGACAATGAT | |
| | | CACCAAAGAG ACAGAGACTC CATCAAGAGC TCTTCAGTAT | |
| | | CCCCATCAGC CAGTTTCCGA GGTGCTGAGA AGCACAGAAA | |
| | | CTCCGCAGAC TACTCCTCAG AGAGCAAAAA GCAGAAAACT | |
| | | GAAGAAAAGG AAATTGCAGC TCGTTATGAC AGCGATGGTG | |
| | | AGAAAAGTGA TGACAACTTG GTGGTTGACG TTTCCAATGA | |
| | | GGATCCATCT TCCCCTCGAG GGAGCCCAGC ACATTCCCCC | |
| | | AGAGAGAATG GCCTAGACAA GACACGCCTG CTCAAGAAAG | |
| | | ATGCCCCGAT TAGTCCAGCC TCTATTGCAT CTTCCAGCAG | |
| | | TACTCCCTCC TCCAAATCCA AAGAACTTAG CCTTAAGAGG | |
| | | GATATGGGGA AATTGAGTGA AACACGTCTT AGCGAAGATG | |
| | | AACAATGCAC ATTGGGGTTA CAGAGATGGT TTTGTCGCCT | |
| | | GTGGTTTATG AATGAAAAAT CTACTACTCC CGTCTCAAAG | |
| | | TCCAATACCC CTACTCCACG AACTGATGCG CCCACCCCAG | |
| | | GCAGTAACTC TACTCCCGGA TTGAGGCCTG TACCTGGAAA | |
| | | ACCACCAGGA GTTGACCCTT TGGCCTCAAG CCTAAGGACC | |
| | | CCAATGGCAG TACCTTGTCC ATATCCAACT CCATTTGGGA | |
| | | TTGTGCCCCA TGCTGGAATG AACGGAGAGC TGACCAGCCC | |
| | | CGGAGCGGCC TACGCTGGGC TCCACAACAT CTCCCCTCAG | |
| | | ATGAGCGCAG CTGCTGCCGC CGCCGCTGCT GCTGCTGCCT | |
| | | ATGGGAGATC ACCAGTGGTG GGATTTGATC CACACCATCA | |
| | | CATGCGTGTG CCAGCAATAC CTCCAAACCT GACAGGCATT | |
| | | CCAGGAGGAA AACCAGCATA CTCCTTCCAT GTTAGCGCAG | |
| | | ATGGTCAGAT GCAGCCTGTC CCTTTTCCAC CCGACGCCCT | |
| | | CATCGGACCT GGAATCCCCC GGCATGCTCG CCAGATCAAC | |
| | | ACCCTCAACC ACGGGGAGGT GGTGTGCGCG GTGACCATCA | |
| | | GCAACCCCAC GAGACACGTG TACACGGGTG GGAAGGGCTG | |
| | | CGTCAAGGTC TGGGACATCA GCCACCCAGG CAATAAGAGT | |
| | | CCTGTCTCCC AGCTCGACTG TCTGAACAGG GATAACTACA | |
| | | TCCGTTCCTG CAGATTGCTC CCTGATGGTC GCACCCTAAT | |
| | | TGTTGGAGGG GAAGCCAGTA CTTTGTCCAT TTGGGACCTG | |
| | | GCGGCTCCAA CCCCACGCAT CAAGGCAGAG CTGACATCCT | |
| | | CGGCCCCCGC CTGCTATGCC CTGGCCATCA GCCCCGATTC | |
| | | CAAGGTCTGC TTCTCATGCT GCAGCGACGG CAACATCGCT | |
| | | GTGTGGGATC TGCACAACCA GACCTTGGTG AGGCAATTCC | |
| | | AGGGCCACAC AGATGGAGCC AGCTGTATTG ACATTTCTAA | |
| | | TGATGGCACC AAGCTCTGGA CAGGTGGTTT GGACAACACG | |
| | | GTCAGGTCCT GGGACCTGCG CGAGGGGCGG CAGCTGCAGC | |
| | | AGCACGACTT CACCTCCCAG ATCTTTTCTC TGGGCTACTG | |
| | | CCCAACTGGA GAGTGGCTTG CAGTGGGGAT GGAGAACAGC | |
| | | AATGTGGAAG TTTTGCATGT CACCAAGCCA GACAAATACC | |
| | | AACTACATCT TCATGAGAGC TGTGTGCTGT CGCTCAAGTT | |
| | | TGCCCATTGT GGCAAATGGT TTGTAAGCAC TGGAAAGGAC | |
| | | AACCTTCTGA ATGCCTGGAG AACACCTTAT GGGGCCAGTA | |
| | | TATTCCAGTC CAAAGAATCC TCATCGGTGC TTAGCTGTGA | |
| | | CATCTCCGTG GACGACAAAT ACATTGTCAC TGGCTCTGGG | |
| | | GATAAGAAGG CCACAGTTTA TGAAGTTATT TATTAAAGAC | |
| | | AAATCTTCAT GCAGACTGGA CTTCTCCTCC TGGTAGCACT | |
| | | TTGCTCTGTC ATCCTTTTTG TTCACCCCCA TCCCCGCATC | |
| | | TAAAACCAAG GATTTCAGAT ACTCATTGCA GTTGTGGAGT | |
| | | TTAATCCCCT TTCTTAACCT CACTTCCCAC TTGCTATTGA | |
| | | ATTGTGAATA GTCATTAAAA ACCTGTGATA CCAAATCTTC | |
| | | AGCTGTCTAC TTGGAAGAAC ATGGAATAAG CATACTTAAC | |
| | | AGTGAAAAGA ATCTTTAATT ATGTATTATA TCTGTAATAT | |
| | | ATTTATTTTG TTTAAAGAAG GCTTTCTAAC AATGACTGAC | |
| | | TAAATAAAGC TGTCTGCTCC TGCATTGATA ATGAAGGTGC | |
| | | GTTGTATTTG ATACCCCTCC CCCCCTTTTT TTGGCAAAGG | |
| | | AGGGGAAAGG AAGGTTTAAA ATAATTGATT TAAAATGTCA | |
| | | CTAAGTGTAG ACTGATGACT GTATAGAGAT GTGAAATGTA | |
| | | TAATTACACA TGGAAGCAAT ATGTTGCTGT GTTGTTATTA | |
| | | GGTTTTTTTT GTTTTTGTTT TCTACATCTT TTAAAGACTT | |
| | | TTGGAAATTT GGCTGAACAA TTAGAACACA ACAGGCCAAC | |
| | | TCATACTCAT TTGGATCTAT TTAGACAACG TTAACCAATA | |
| | | TATCTATAGC TTTAGATTAT ATTCGATAAA AGTAATTGGA | |
| | | CTTTTTTTCT TTTTTTGACT CGTTGACAAG TGTCTTTGTA | |
| | | ATATGTTTTT AGTTCCCTTT TTTTGTTGTA TTATAGGCAG | |
| | | ATGAACAAAT TAAATTTGGC CTCAAAGAGA GAACTTACTC | |
| | | CCTTCTGGAT ATTTTTGCCA CATTTCTTTG CAAAAGGAGA | |
| | | TATATATATC TTTAGTCAGT TTTGTTGTTA TGAGAAATTA | |
| | | TGGGTTATTT TGTGGCATGC TCTTTGGGAG CTGCACAGTT | |
| | | ATGGGGAGGA CTCCCACTGC TGTGCAAGTT AAGTCTTTTA | |
| | | CAAAACAAGG ACAGCAGAGG AGGGTTTGCA GAGACCTCCC | |
| | | TCTGAAAAAC ACAAAGAATG GACTCTCTCC TGGGATGAGG | |
| | | ACTTGCTTTC TTTACCTCCG GTTCTTTCCA TGTCTTAGTT | |
| | | GGATGTCCCT GAAATGGACA CAGGCTGTGC CATTGTGCCA | |
| | | GAAACATTGT GTTATCTTTT ATGTTGTTGT TGTTGCTGTT | |
| | | AAACTATAAT ATGTGACTTC TTTTTTTATT ATTTTTTGTT | |
| | | TGAATGCTTT AAAAATCTTT TAAGTCTGTG GATCTGCTGA | |
| | | TGTACAGTGC CTTTGCTGCT ATGGATCAAA ATCAAAAGAA | |
| | | CCGTGTAGAT ATACTTTATT GTATAAGTAG AAAATTACTT | |
| | | AATTTCATAC TAGAAATGGA TGGATGCTGC AAGTTGAAAT | |
| | | GGACTGTCCA TTGACGTTCC TAATGTGGTA GCAGAAAAAA | |
| | | AAAAATGGTG TCTTAAGTGC TTAGTGTTTG ATGTCATTAA | |
| | | CAGTTTCGTA AAACTCTACA GTGTAGAAAG ATTTTGATAC | |
| | | TAAACTGTGC GTTGTACATA GTTCTAATGC ATTGTATTGA | |
| | | CCACCAGTAC TTCTATAATG GTAGATTGTT TGTGAATTCA | |
| | | GACTTTTAAG CATTAAACAT AAATAACTTC TAGTATGCTT | |
| | | ATTTTTCTAA TTCTTTGTCT TGATGACATT AGTTTATTTT | |
| | | TTATCTTTGG CTGTGCCACT CCTATATATT AAAAATGCCT | |
| | | AGTTTTTTCA AGGGAGATTG TTGTTAAAGT AAAGTGGTTT | |
| | | TTTTTGTTGT TAAA | |
| TPT1 | NM_001286272.1 | CTTCGTGCCA CGTCACCGCC TGCGTCGCTT CCGGAGGCGC | 25 |
| | | AGCGGGCGAT GACGTAGAGG GACGTGCCCT CTATATGAGG | |
| | | TTGGGGAGCG GCTGAGTCGG CCTTTTCCGC CCGCTCCCCC | |
| | | CTCCCCCCGA GCGCCGCTCC GGCTGCACCG CGCTCGCTCC | |
| | | GAGTTTCAGG CTCGTGCTAA GCTAGCGCCG TCGTCGTCTC | |
| | | CCTTCAGTCG CCATCATGAT TATCTACCGG GACCTCATCA | |
| | | GCCACGATGA GATGTTCTCC GACATCTACA AGATCCGGGA | |
| | | GATCGCGGAC GGGTTGTGCC TGGAGGTGGA GGGGAAGATG | |
| | | GTCAGTAGGA CAGAAGGTAA CATTGATGAC TCGCTCATTG | |
| | | GTGGAAATGC CTCCGCTGAA GGCCCCGAGG GCGAAGGTAC | |
| | | CGAAAGCACA GTAATCACTG GTGTCGATAT TGTCATGAAC | |
| | | CATCACCTGC AGGAAACAAG TTTCACAAAA GAAGCCTACA | |
| | | AGAAGTACAT CAAAGATTAC ATGAAATCAA TCAAAGGGAA | |
| | | ACTTGAAGAA CAGAGACCAG AAAGAGTAAA ACCTTTTATG | |
| | | ACAGGGGCTG CAGAACAAAT CAAGCACATC CTTGCTAATT | |
| | | TCAAAAACTA CCAGTTCTTT ATTGGTGAAA ACATGAATCC | |
| | | AGATGGCATG GTTGCTCTAT TGGACTACCG TGAGGATGGT | |
| | | GTGACCCCAT ATATGATTTT CTTTAAGGAT GGTTTAGAAA | |
| | | TGGAAAAATG TGATGCAAAA GAAAGAAATC CCTGCGCTTT | |
| | | CTGTCTGTCT TTGTGGCGGC CCAGATTGAA TTGGGGAATA | |
| | | CATCTTTAGC CTGGAAATGT AGGCTGCATG TTAATGGTAA | |
| | | TGTAACTTTT GCAGTGTAAT GTTTGAAAAA TATTAATGTA | |
| | | GTTTTTGCTT TTACAGTAAC AAATGTGGCA ATTATTTTGG | |
| | | ATCTATCACC TGTCATCATA ACTGGCTTCT GCTTGTCATC | |
| | | CACACAACAC CAGGACTTAA GACAAATGGG ACTGATGTCA | |
| | | TCTTGAGCTC TTCATTTATT TTGACTGTGA TTTATTTGGA | |
| | | GTGGAGGCAT TGTTTTTAAG AAAAACATGT CATGTAGGTT | |
| | | GTCTAAAAAT AAAATGCATT TAAACTCATT TGAGAGAATG | |
| | | CCTTTTAGTT TAATGCATAT TTAAACTAAA TTGATCCTGT | |
| | | AGTGTTCCTG GAGAAGCTAG AGCCTGATTG TAGGCTACTA | |
| | | CTCATCAATT AACTTCTACA GTGGAGACTA CTTCTGGGAC | |
| | | TGGAATATAA AAAAGAATCA AAGGTTCTGA TTTTGAGTTG | |
| | | CAATAAAGGG AAAGACCATG CTCATAGCAG TGCCAACATC | |
| | | TGAAGTGTGG AGCCTTACCC ATTTCATCAC CTACAACGGA | |
| | | AGTAGTTAAC TGGAAGAGAT TACCAAGAGA ATAAAAAGAG | |
| | | ACTCATTCAG TGGAAGCAAC TTTGTCTCAG CTTATTTCAC | |
| | | ATAAAGAGAG CGAAGTCTTT TGGGATGAAT GTTAATTAAA | |
| | | CTCCCTGGTA ACTAGAACAG GGACTGGCAA ACTAGCCTAT | |
| | | CTGACCACCT GTTTTGTACA CTTTAAGGTG GTTGGTTGCC | |
| | | TTTTTAAATG GTTGAGGGGA AAAGAATACC TTGTGGGATA | |
| | | TGGAATTTAA GTTCGAGTCC AGTTTTATTG GAACGTGGCT | |
| | | ATGCTTATTC ATTTATGGAT TGACTGTGGC TGTTGTCAGT | |
| | | GCATGAGCAG AGTTGTGTCT AACAGACTAG AGCCTGCAAG | |
| | | TTTGCCAGCC CCTGATTTAA AAGATGAAGG TACACAGAAT | |
| | | GTGGGCTGGC TGGTGGGCAA AGGGGTAAAA ATGTTCTCTA | |
| | | TATTGTATCT GAAAAGATGG GGTGTCTGAA TAAGAAAATG | |
| | | CATCTATTTG ACAGACCTGG AGCAGTTGCT ATCTGCTGCT | |
| | | ATGGTTTCCA CCACAGATGC AAGAAGAACA TGTCCTTGCG | |
| | | CTTTCCGTCT GTCTAATTGT GGCAGCTGAG ATTGAATAGA | |
| | | GGAATACAGG AGGAAAAAAA GCGGGAAGAG TTTTTGAGGC | |
| | | AGGTCGGTCA CCCAGGCTTG TAGTGCAGTG GCACAAGCAA | |
| | | CTCACTGCAT TCTCTGCATC CTGTGCTCAA GCCATTTTCC | |
| | | CACCTCAGTC TCACTAGTTG CTGGGACTGC AGGCATGCAC | |
| | | CCCTATGCCC AGCTAATTTT TGTAGAGACC GAGTATCGCT | |
| | | TAGTTGCCCA GGGTGGTCTC AACTCCTGGG CTCAAGGAGA | |
| | | TCTGCCCACC TCAGCCTCCC AAAGTGCAGG CCTAGCCTGG | |
| | | GAGGGGAATT TTCAAAACGT GAGTTTTGGG AAATAGTCTA | |
| | | TCAGCCTTAC CTGGTTGATT ACACTTGTAA AAGAAAGATT | |
| | | AAAAGCAGGC CAGTGACTCT GGTCTGCTTG AACATGTGAA | |
| | | TGTAGTGGTT TGAGCAATCT GGAGTTTGCC CTAGTGTCAA | |
| | | ATTCCAGACT GTCCATAGTG TCCAAAACCT GAGGCAGATA | |
| | | CTAATGTTAA CCCCCAGCAC CCCGTGATTG GAAACAAACC | |
| | | TAAATACGTA TTGGGAACTT AATAGCAATT TTAAGCATTC | |
| | | TGATAGATTT TTTGTAGGGA TGGGGTCATG CCATGTGGCC | |
| | | CAGGCTGGTC TGAAAACTCT GGCCTCAAGT GATCTCAAGC | |
| | | TTTGGCCTTC TAAAGTGTTG GGATTACAGG TGTGAGGCAT | |
| | | TGCACCTGGC TTAGCGTTCT GATTTGACAT TGTAATGAAA | |
| | | AGTGTGAGTC TCATCTACAG GGCCTTTTGT CCTCTGAAAT | |
| | | GATAGCAGGA AGGGAATTTT CAGGCAGTGG TCAAAGCTGG | |
| | | GGAAACCAGG ATAGTGAAGA AGGCCTTGAG GTGAGAGATG | |
| | | GAAGCTAATT GGTGAACTAG CCTTGGAAGC CTGAAACAGA | |
| | | CAAGTAGCAA TTCAGAGACT TTGTGGGCTC CACTGCTCCA | |
| | | ACTTGTTTTG AAGATTTTCA GTTCTGCAGA AGAGGTATTT | |
| | | CCCCAGTTGT CCTTTCAGTG CTCTTAGCTG TTTTCCCAAC | |
| | | ATCCAGATCC AATCAAGGCT GGGACATAGC ATTTTATCAT | |
| | | GTCTATTTAA GTCAGAAGTG ATGAACCCCA GCTGTTTACC | |
| | | TCATGGTAAA CCTTTGAAGA TTCCAGGTAG AATCTTCTCA | |
| | | GACTTTGAAG ACTGTCTCAT TTTATATCTT TTTCTCGTTA | |
| | | TTCCTAGGGT CAAGACGTTT TGGGCAAGAA TAAGGATGTG | |
| | | AACATCAGAA AGCTCATAAC ATTTTGTTTT TGATGCTAAG | |
| | | TTTAACAAAG GCATGCTTTA GTAGCCTGTG GGCCCTAGGG | |
| | | TTTGTTAAAG TGTGGAGAAC AACTGAGTGG AGCAAGAGGA | |
| | | CTTTTCTAGG AAGGTCCTTG TAATGTGACA TTTGAAAACA | |
| | | AATGAAGGTG TGGAAGTAGG CCATGTGGAT ATCAGGACAA | |
| | | ACCATTCCAG GCCAAGACAA CAGCAGTTAG TCTGGAGTGT | |
| | | GATGTGTTCT GGGAAAAAAG TGGCCACTTT GCTAACCCAA | |
| | | GAAGACAGGA AGGGTTGTAA AGCAGTGGGA GTGTGCAAGG | |
| | | AAGGAAGACC AGACCTCAAG GAAACCACAG GCGCTCTGAG | |
| | | CAGAAGAGTT ACATGATATG ACTCAAATTT TTAAAGGATC | |
| | | ACTTTGGCTG CCAGGTGGCA GGGTAAAAGC ATAGAATAAT | |
| | | TGTGTATAAT GTGTTTTTAA GGCAAAGATA GTGGCTTAGT | |
| | | CTAGGGTAGT AGACTGAGGT GGTAGGAAAT GAAGATAGAG | |
| | | ACAACAGGAT ATGCTGGTGG GTGAGGATGG ATTTAATGTT | |
| | | GATACAAGTA TTTTGGTCTG AGCGTTTGGA AGAAAGTTGG | |
| | | CACTGAGGTG GGAAGTCGAG TTTAGTTTTG TTAGTTTTGG | |
| | | ATGTGTTAAG TTTGAGATGC TGATTCTTCA GAGAAGTCTA | |
| | | AGCTGGAGAA CTATATAGAG AGTGGAAAGA TAACAATAGA | |
| | | CATTGAAAGC CATGATACAG GATAAGGTCA TTTGGAGAGA | |
| | | GGATAGACTG CATTCCAACA TGAGATTGGT TGACAAAGAG | |
| | | AAACCAACAA AGGTAATTAA GAGGTGCTCC CACTGCACTT | |
| | | GTACTCAGAA GGCTGAGGTA GGATTGTTAG AGGCCAGCCT | |
| | | GGGCACCACA GGGAGACCCC ATCTCTAAAA TTTAGCCAGG | |
| | | AACCATGGCT CATGCCTGTA GCCCCAGGAA TTTGGGAGGC | |
| | | TGAGTGGGGA GGATCGCTTG AGGTCAGGAG TTTGAGACCA | |
| | | GCCTGGGCAA CATAGGGAGA CCTAAAAAAA TTAATTGGGC | |
| | | ATCTGTAGTC CCAGCTACTC AGGCGGCTGA GCTGAGAGGA | |
| | | TGGCTTGAGT CCGAGAGATT GAGGGTGCAG TGAGCTGTGA | |
| | | TCATACCACT GCACTCCAGC CTGGGCGGCA GTGAGACACT | |
| | | ATCTGAAAAA AGTTTAAAAA TTTTAAAAAA GAAGGAACTG | |
| | | CCCCTGAGGT AAGAACCAAG GGAGGGCCTC CCAGAGGTCA | |
| | | GGTGGAAAAA GTTTTAGGAA GGAGGAAGTA GTCAACAGGG | |
| | | TTACCTGTTG CAAAGTACTT AAGTAATATG AGGCCTGATA | |
| | | GTGGTAAACT TGACTACCGT TGGATTTCAC TAGTGGGAAA | |
| | | GGAAGTCTAA TTAAAATGCA CTCAAGAGAC TAACAGTCGC | |
| | | AGGCATGAAA TACAATACAG GTACATGGTT TTTTATTATG | |
| | | TGTGCATCTG CTTCAGTAAT AGGTGTGAAT TACTCATTTG | |
| | | GATCATTAGG AGTTTCAAAA TCTAGTTAAA TGACTAGATT | |
| | | TTTGTTGATG TAAATTCTGT CATTCTGAAC TGCAGGGATT | |
| | | GTCAGTAACT TAACTGCAAA CTAAACTGGT GATAATTATG | |
| | | GTAAAATTGC AAGACGAGCA ATAAATCTCA ACCAACTTGA | |
| | | GAGAACACTG ATAA | |
| TSC22D3 | NM_001015881.1 | CGCCGTGAAA ACCAGCTGCT GCAGCCGCAG AGGCAGCCGG | 26 |
| | | AGGCAGAGGG GCGGCGGGCA GGACCAGACA GGGCTGGGCA | |
| | | GGGGGCTGGC CGAGCGCCGT GCGCCGCTTG GGAGAAGGCC | |
| | | GGAAGCTTAC CAGCCGAGAA GGAATTCCTA GCTAGCTTCA | |
| | | GAGCCGGTGC CTCCGGAGCC AGCGTGGTGG CCATAGACAA | |
| | | CAAGATCGAA CAGGCCATGG ATCTGGTGAA GAATCATCTG | |
| | | ATGTATGCTG TGAGAGAGGA GGTGGAGATC CTGAAGGAGC | |
| | | AGATCCGAGA GCTGGTGGAG AAGAACTCCC AGCTAGAGCG | |
| | | TGAGAACACC CTGTTGAAGA CCCTGGCAAG CCCAGAGCAG | |
| | | CTGGAGAAGT TCCAGTCCTG TCTGAGCCCT GAAGAGCCAG | |
| | | CTCCCGAATC CCCACAAGTG CCCGAGGCCC CTGGTGGTTC | |
| | | TGCGGTGTAA GTGGCTCTGT CCTCAGGGTG GGCAGAGCCA | |
| | | CTAAACTTGT TTTACCTAGT TCTTTCCAGT TTGTTTTTGG | |
| | | CTCCCCAAGC ATCATCTCAC GAGGAGAACT TTACACCTAG | |
| | | CACAGCTGGT GCCAAGAGAT GTCCTAAGGA CATGGCCACC | |
| | | TGGGTCCACT CCAGCGACAG ACCCCTGACA AGAGCAGGTC | |
| | | TCTGGAGGCT GAGTTGCATG GGGCCTAGTA ACACCAAGCC | |
| | | AGTGAGCCTC TAATGCTACT GCGCCCTGGG GGCTCCCAGG | |
| | | GCCTGGGCAA CTTAGCTGCA ACTGGCAAAG GAGAAGGGTA | |
| | | GTTTGAGGTG TGACACCAGT TTGCTCCAGA AAGTTTAAGG | |
| | | GGTCTGTTTC TCATCTCCAT GGACATCTTC AACAGCTTCA | |
| | | CCTGACAACG ACTGTTCCTA TGAAGAAGCC ACTTGTGTTT | |
| | | TAAGCAGAGG CAACCTCTCT CTTCTCCTCT GTTTCGTGAA | |
| | | GGCAGGGGAC ACAGATGGGA GAGATTGAGC CAAGTCAGCC | |
| | | TTCTGTTGGT TAATATGGTA TAATGCATGG CTTTGTGCAC | |
| | | AGCCCAGTGT GGGATTACAG CTTTGGGATG ACCGCTTACA | |
| | | AAGTTCTGTT TGGTTAGTAT TGGCATAGTT TTTCTATATA | |
| | | GCCATAAATG CGTATATATA CCCATAGGGC TAGATCTGTA | |
| | | TCTTAGTGTA GCGATGTATA CATATACACA TCCACCTACA | |
| | | TGTTGAAGGG CCTAACCAGC CTTGGGAGTA TTGACTGGTC | |
| | | CCTTACCTCT TATGGCTAAG TCTTTGACTG TGTTCATTTA | |
| | | CCAAGTTGAC CCAGTTTGTC TTTTAGGTTA AGTAAGACTC | |
| | | GAGAGTAAAG GCAAGGAGGG GGGCCAGCCT CTGAATGCGG | |
| | | CCACGGATGC CTTGCTGCTG CAACCCTTTC CCCAGCTGTC | |
| | | CACTGAAACG TGAAGTCCTG TTTTGAATGC CAAACCCACC | |
| | | ATTCACTGGT GCTGACTACA TAGAATGGGG TTGAGAGAAG | |
| | | ATCAGTTTGG GCTTCACAGT GTCATTTGAA AACGTTTTTT | |
| | | GTTTTGTTTT GTAATTATTG TGGAAAACTT TCAAGTGAAC | |
| | | AGAAGGATGG TGTCCTACTG TGGATGAGGG ATGAACAAGG | |
| | | GGATGGCTTT GATCCAATGG AGCCTGGGAG GTGTGCCCAG | |
| | | AAAGCTTGTC TGTAGCGGGT TTTGTGAGAG TGAACACTTT | |
| | | CCACTTTTTG ACACCTTATC CTGATGTATG GTTCCAGGAT | |
| | | TTGGATTTTG ATTTTCCAAA TGTAGCTTGA AATTTCAATA | |
| | | AACTTTGCTC TGTTTTTCTA AAAATAAAAA AAAAAAAAAA | |
| | | AAAAAAAAAA AAAAAAAAAA A | |
| UBE2J1 | NM_016021.2 | GCGGCCGCGG CAGGGCTGGG CCTGCGACTA CCCGAGGAGG | 27 |
| | | CTGACCTCCA GCCCGGGCGC CCGGTTCAGC GCCGCCCCGG | |
| | | CCGGCGCCGG TGCCTGCCAG GCACTCAGGG AGGCGGGGGC | |
| | | GCAGTGGAGG AGGCGGCGCC ATCGCGAAGC GAGCGCCTCG | |
| | | CCCGCACTCA GCCTTGCCAC CCCGCCCGCA GTCCAGGCTG | |
| | | GACTGGGCGG CATTTGCCGA GGCTCCTCGG CCAGGCCCCG | |
| | | TCCGCCCGAG CCGCGCTGAG ACCCGGGCAG CGGCCGCGTG | |
| | | GAGAGGAGGT GGCAGCGGCC CGGGAGGCCG GAGCCAAGCC | |
| | | AGCGACCCAC CATGGAGACC CGCTACAACC TGAAGAGTCC | |
| | | GGCTGTTAAA CGTTTAATGA AAGAAGCGGC AGAATTGAAA | |
| | | GATCCAACAG ATCATTACCA TGCGCAGCCT TTAGAGGATA | |
| | | ACCTTTTTGA ATGGCACTTC ACGGTTAGAG GGCCCCCAGA | |
| | | CTCCGATTTT GATGGAGGAG TTTATCACGG GCGGATAGTA | |
| | | CTGCCACCAG AGTATCCCAT GAAACCACCA AGCATTATTC | |
| | | TCCTAACGGC TAATGGTCGA TTTGAAGTGG GCAAGAAAAT | |
| | | CTGTTTGAGC ATCTCAGGCC ATCATCCTGA AACTTGGCAG | |
| | | CCTTCGTGGA GTATAAGGAC AGCATTATTA GCCATCATTG | |
| | | GGTTTATGCC AACAAAAGGA GAGGGAGCCA TAGGTTCTCT | |
| | | AGATTACACT CCTGAGGAAA GAAGAGCACT TGCCAAAAAA | |
| | | TCACAAGATT TCTGTTGTGA AGGATGTGGC TCTGCCATGA | |
| | | AGGATGTCCT GTTGCCTTTA AAATCTGGAA GCGATTCAAG | |
| | | CCAAGCTGAC CAAGAAGCCA AAGAACTGGC TAGGCAAATA | |
| | | AGCTTTAAGG CAGAAGTCAA TTCATCTGGA AAGACTATCT | |
| | | CTGAGTCAGA CTTAAACCAC TCTTTTTCAC TAACTGATTT | |
| | | ACAAGATGAT ATACCTACAA CATTCCAGGG TGCTACGGCC | |
| | | AGTACATCGT ACGGACTCCA GAATTCCTCA GCAGCATCCT | |
| | | TTCATCAACC TACCCAACCT GTAGCTAAGA ATACCTCCAT | |
| | | GAGCCCTCGA CAGCGCCGGG CCCAGCAGCA GAGTCAGAGA | |
| | | AGGTTGTCTA CTTCACCAGA TGTAATCCAG GGCCACCAGC | |
| | | CAAGAGACAA CCACACTGAT CATGGTGGGT CAGCTGTACT | |
| | | GATTGTCATC CTGACTTTGG CATTGGCAGC TCTTATATTC | |
| | | CGACGAATAT ATCTGGCAAA CGAATACATA TTTGACTTTG | |
| | | AGTTATAATA TGGTTTTGTG ACTTATGAGC TGTGACTCAA | |
| | | CTGCTTCATT AAACATTCTG CATTGGGTAT AATCTAAGAA | |
| | | TTGTTTACAA AAAGATTATT TTGTATTTAC CCTTCATTCC | |
| | | TTTTTTTGAT CCTTGTAAGT TTAGTATAAA TATATCTAGA | |
| | | CATTCAGACT GTGTCTAGCA GTTACGTCCT GCTTAAAGGG | |
| | | ACTAGAAGTC AAAGTTCCTT GTCTCACTAT TTGATCTGCT | |
| | | TTGCAGGGAA ATAACTTGTT TTTTCTCATG TTTCATCTTC | |
| | | TTTTTATGTA AATTTGTAAT ACTTTCCTAT ATTGCCCTTT | |
| | | GAAATTTTTG GATAAAAGAT GATGTTTTAA GTTCCAATGA | |
| | | GTATTACTAG TTACTCAATA CCACTTATTG AGTACTCTGT | |
| | | TTCTACGTAT GTAGAATGTA TAGGGATAGA AGAGTTGAAA | |
| | | AGGGAAAGCA AAACTCCTCA AGTAGCTTCC TTAAAATGTC | |
| | | ATTCATAGGA GATGTACTGG AATTGCTCAT TCTGTGACTT | |
| | | TATTTGTGTC CTAAACATTC TTCAGTGAAA ATAATTTTAT | |
| | | TTCAGTCAAA CATTTATGAG GAAATGAGAT CACATCTTTG | |
| | | TCACTGGATG CTACTTGAAG AGGGAGTACT TTGTAACCAC | |
| | | TTTGATATGC TGTTATCACC ACCCCCTGCC CTCTGCTGCC | |
| | | ATAATCACAC AAATTTAAAA AGAAAGAAAA CAGTCTTCCA | |
| | | TAGATTTTTA AGGAAGAAAG GGCCCAAGCC AGGAGATCGC | |
| | | TTGGTTTTCT TCCAGAAGTT AAATGGGGGG ATCTGAAGAT | |
| | | TTGAATGTTT GGTCTGCTTT GAAATGTATG TCTTTTGGGA | |
| | | TGTATTATAT GCCTAGCTTT ATAATCAGTA TAAATTTTAA | |
| | | TTATTCCAGG AATATGCATA ATATTGAAAT ATTTCATGTC | |
| | | CTATTTTAAT AGAAAACCTC AGGGCCCAAG TAACAGTGAT | |
| | | AGAAGTTAGA AAAACCTTTA CTTAGAATTG TCCACCTAGT | |
| | | CAGAGCCCAA GAAAGAATTT TCAGTGGAAA AATCAATATA | |
| | | TAACTTAGTG CTAGCTAGCG CCACAGACTC TAGTAGATAA | |
| | | TATTATCATC ATAATGGCTG GTGAAACCAT ATAATCACAG | |
| | | AAAAACATTG CCTTCAGCAT GTTCAGTTCG CAGCACTGAG | |
| | | GGCACTCTTG AGGGTGTTGT TAATGAAGAT TTAATTTTTA | |
| | | AATACAGGTG GTTCCAAGCT TTCAAATAGG TTATGCTCCA | |
| | | AAAGTGTTAT TTGTAAGTTA ATTTTTTTAC AAGTCAAACA | |
| | | ATGTTGGAAG TGGTATTTAG GTTCTAGATC GGTCCACGAA | |
| | | AGTTAGCCCA TATGTATATC TTGAATAGTA TAGGGGAGGG | |
| | | TATTCATAAA GTCCTTATGT GGTTTTAACT AAGTGAAATT | |
| | | ATGGACAAGA GAAATAATTG TAAAATCGTC TTAAAGGAAA | |
| | | ATTTAATTTT TACTCCTGTT TATGGGACAT TCGTTCTATT | |
| | | AACTGTCAGA CACAATTTCT GTTTTCATCT GAGAGCCAGT | |
| | | TTTCCTTTAT TTCTACATCT AAAATAAGAA CATATTGTAC | |
| | | ACTATTATAT AATACAGAAT TGTCTTAAAC TTTAATAAAT | |
| | | TCGCATTTTA AAGGTGTTTA CAGATTATTT TTTATATCTG | |
| | | TAGCTGAATT TGTTAAAGTC TAAAAAGCTC AAGGACTTTA | |
| | | TGAAGATCTC ATTATATGAG GAAAATCATA GGTTACCATT | |
| | | TTATAACTCT ATTGCCATAA GAAAATACAC TCTAAAATCT | |
| | | TGATTTGAAA CATATTAGAA ACCTTGATTC AGTGCTCAGT | |
| | | GGTCTCCTAG TAAGAAGTCA CCGACGGTAG CGTCATATGA | |
| | | GAAGAAAGAA ATCCCCACCA CCTCAACCTC TGCTGAGATT | |
| | | GTGTGCTAGG AACAGCCTTC CCTCCGTTTC CCCTCAGTCA | |
| | | AACTTGAGCC AGCCTCTGGA TCGATGTGAT CTTATTGCAT | |
| | | GTTTCCATGG GGTGTACCTA TACTTTAAGC CAATCCTGCT | |
| | | GCATTCACTG CTAAGTTAAA TAAAAAGCCA AGAAGATTTT | |
| | | GCACTGTGCA GATCCTTTGC TATCTGACTT GCATCTCTTC | |
| | | CCCCACCTGT CAGCTAGCCA CCTGCTTGTT TGTGTTGGGA | |
| | | TATTTTTTAG CACCTGAAGC ACCATCTGAA AGGGGCACCA | |
| | | TTTTCTTCTT CCCTTTGATC TCACATATGC TCCCTAAAAA | |
| | | TCCTTAAGTT GTCAATCTGA TCCCCAGTGT GAGGTTAATG | |
| | | AGCAAAATTG GTCTTTGGGG CCCTTTTTGT CCAAGCCCCA | |
| | | CTGAAAGGCC TCTTCAGAAA ACTATTATCT TTAAAGCCCT | |
| | | ACTTTAACTC CTTAATTCCA GCATACAGCT AAAACTGGAT | |
| | | GTATATTCTG GCAAGTAAAG GCTGAGGACT CCTCTTTAAT | |
| | | CCTCAGATCT AGATAACTCA TGACATTTTA TTTGACCAAC | |
| | | ATAGCACATG ATGAGATATC AAGGTAATTA AAATAGCATG | |
| | | CTTGAAAAAA AAATACGTAA TCTGTTTCAC CTGTAACTGT | |
| | | TTAAGCCAAT AAACTTTTCA AAATTTATGT AATGTGGGGC | |
| | | TTTTATGTAG CACTTTACGT TTTCATGCTG CTTATTGTTT | |
| | | TATTCTACTG AAAAAAATGA ATTTCAAGAT TCTCAACTTT | |
| | | TTTAATTTCA AAAATTGTTT ATTGTTTTGA CTATAGGAAT | |
| | | ACAAAATTTC CTATTTTGGG AGAATAAGAA CTCTTTTTGT | |
| | | CATTTTTGGC TATGAATAAA CTTTCTGGTC TTTTGAGACC | |
| | | ACCCATTTTT ATAGATCAGA ATCAGAAAAC AGGTAAACCT | |
| | | CACTCACACA TTTGGACTCA TTTGAACAAA AATCTAGGCC | |
| | | AAAATACTGA AAAGCCTATG TGTTTTTTTA ATTGGAAGTA | |
| | | TATGTAAGGT TAATGCATTT AGTGAACGTG ACTAACAAAG | |
| | | ACTAATGTGC ACATTAACAG ATGTACTTTT TAAGGTTTTA | |
| | | TGGGAGGCTG TGCATTGCTC AAAAGCTGTT GGGAACGCCT | |
| | | TCTGAACAGT TGCCTTCAGA ACTAGTTTGA GCTGCTCAAT | |
| | | AAAACCAGTG ACTTTACTCA TAAAAAAAAA AAAAAAAAAA | |
| ALG9 | NM_001077690.1 | GTCTTTTGTC CCTCGGCGGA CACCGTTTGC CAGCCAAAGC | 28 |
| | | TATGTCTGCG CGCTCACCGA CTTCATAGGG TGCCGAATTC | |
| | | TTTTTTCCCC AGGCTTGCCA TGGCTAGTCG AGGGGCTCGG | |
| | | CAGCGCCTGA AGGGCAGCGG GGCCAGCAGT GGGGATACGG | |
| | | CCCCGGCTGC GGACAAGCTG CGGGAGCTGC TGGGCAGCCG | |
| | | AGAGGCGGGC GGCGCGGAGC ACCGGACCGA GTTATCTGGG | |
| | | AACAAAGCAG GACAAGTCTG GGCACCTGAA GGATCTACTG | |
| | | CTTTCAAGTG TCTGCTTTCA GCAAGGTTAT GTGCTGCTCT | |
| | | CCTGAGCAAC ATCTCTGACT GTGATGAAAC ATTCAACTAC | |
| | | TGGGAGCCAA CACACTACCT CATCTATGGG GAAGGGTTTC | |
| | | AGACTTGGGA ATATTCCCCA GCATATGCCA TTCGCTCCTA | |
| | | TGCTTACCTG TTGCTTCATG CCTGGCCAGC TGCATTTCAT | |
| | | GCAAGAATTC TACAAACTAA TAAGATTCTT GTGTTTTACT | |
| | | TTTTGCGATG TCTTCTGGCT TTTGTGAGCT GTATTTGTGA | |
| | | ACTTTACTTT TACAAGGCTG TGTGCAAGAA GTTTGGGTTG | |
| | | CACGTGAGTC GAATGATGCT AGCCTTCTTG GTTCTCAGCA | |
| | | CTGGCATGTT TTGCTCATCA TCAGCATTCC TTCCTAGTAG | |
| | | CTTCTGTATG TACACTACGT TGATAGCCAT GACTGGATGG | |
| | | TATATGGACA AGACTTCCAT TGCTGTGCTG GGAGTAGCAG | |
| | | CTGGGGCTAT CTTAGGCTGG CCATTCAGTG CAGCTCTTGG | |
| | | TTTACCCATT GCCTTTGATT TGCTGGTCAT GAAACACAGG | |
| | | TGGAAGAGTT TCTTTCATTG GTCGCTGATG GCCCTCATAC | |
| | | TATTTCTGGT GCCTGTGGTG GTCATTGACA GCTACTATTA | |
| | | TGGGAAGTTG GTGATTGCAC CACTCAACAT TGTTTTGTAT | |
| | | AATGTCTTTA CTCCTCATGG ACCTGATCTT TATGGTACAG | |
| | | AACCCTGGTA TTTCTATTTA ATTAATGGAT TTCTGAATTT | |
| | | CAATGTAGCC TTTGCTTTGG CTCTCCTAGT CCTACCACTG | |
| | | ACTTCTCTTA TGGAATACCT GCTGCAGAGA TTTCATGTTC | |
| | | AGAATTTAGG CCACCCGTAT TGGCTTACCT TGGCTCCAAT | |
| | | GTATATTTGG TTTATAATTT TCTTCATCCA GCCTCACAAA | |
| | | GAGGAGAGAT TTCTTTTCCC TGTGTATCCA CTTATATGTC | |
| | | TCTGTGGCGC TGTGGCTCTC TCTGCACTTC AGAAATGTTA | |
| | | CCACTTTGTG TTTCAACGAT ATCGCCTGGA GCACTATACT | |
| | | GTGACATCGA ATTGGCTGGC ATTAGGAACT GTCTTCCTGT | |
| | | TTGGGCTCTT GTCATTTTCT CGCTCTGTGG CACTGTTCAG | |
| | | AGGATATCAC GGGCCCCTTG ATTTGTATCC AGAATTTTAC | |
| | | CGAATTGCTA CAGACCCAAC CATCCACACT GTCCCAGAAG | |
| | | GCAGACCTGT GAATGTCTGT GTGGGAAAAG AGTGGTATCG | |
| | | ATTTCCCAGC AGCTTCCTTC TTCCTGACAA TTGGCAGCTT | |
| | | CAGTTCATTC CATCAGAGTT CAGAGGTCAG TTACCAAAAC | |
| | | CTTTTGCAGA AGGACCTCTG GCCACCCGGA TTGTTCCTAC | |
| | | TGACATGAAT GACCAGAATC TAGAAGAGCC ATCCAGATAT | |
| | | ATTGATATCA GTAAATGCCA TTATTTAGTG GATTTGGACA | |
| | | CCATGAGAGA AACACCCCGG GAGCCAAAAT ATTCATCCAA | |
| | | TAAAGAAGAA TGGATCAGCT TGGCCTATAG ACCATTCCTT | |
| | | GATGCTTCTA GATCTTCAAA GCTGCTGCGG GCATTCTATG | |
| | | TCCCCTTCCT GTCAGATCAG TATACAGTGT ACGTAAACTA | |
| | | CACCATCCTC AAACCCCGGA AAGCAAAGCA AATCAGGAAG | |
| | | AAAAGTGGAG GTTAGCAACA CACCTGTGGC CCCAAAGGAC | |
| | | AACCATCTTG TTAACTATTG ATTCCAGTGA CCTGACTCCC | |
| | | TGCAAGTCAT CGCCTGTAAC ATTTGTAATA AAGGTCTTCT | |
| | | GACATGAATA CTGGAATCTG GGTGCTCTGG GCTAGTCAAA | |
| | | GTCTATTTCA AAGTCTAATC AAAGTCACAT TTGCTCCCTG | |
| | | TGTGTGTCTC TGTTCTGCAT GTAAACTTTT TGCAGCTAGG | |
| | | CAGAGAAAGG CCCTAAAGCA CAGATAGATA TATTGCTCCA | |
| | | CATCTCATTG TTTTTCCTCT GTTCAATTAT TTACTAGACC | |
| | | GGAGAAGAGC AGAACCAACT TACAGGAAGA ATTGAAAATC | |
| | | CTGGTACTGG ATGGCTGTGA TAAGCTGTTC TCCACACTCT | |
| | | GGCCTGGCAT CTGAGAACTA GCAAGCCTCT CTTAGGCCAT | |
| | | ATGGGCTTCT CCACCAAAGC TGTTTGGCAG CTCCTAGCAG | |
| | | ACCTTCTTAT TGAAATCCTC ATGCTGAAAA TGAACACAGC | |
| | | CTAGTTGCCA ACCCACATGT CCTTTTCACC TCCAGCAAGA | |
| | | CTAAGCTTCT TTAAAGCACT TCACAGGACT AGGACCCTGT | |
| | | CCTGGAGCTA TCTCAGGAAA AAGGTGACCA TTTGAGGAAC | |
| | | TGTGACCTAA TTTTATTATA ATGATGCCTC TAATTTTCAT | |
| | | TTCCTTTACA ACCAACTGTA ACTATAAGGT TGTATTGCTT | |
| | | TTTTGTTCAG TTTTAGCATG CTATTTTTTG AATTCTAGAC | |
| | | TCCTCCATGT GAAGATATCA ACAGACAAAA CTACAACTGT | |
| | | ATAGGACATA TTTGGAGAAA ATTCTATCAA TTGATACATT | |
| | | TGGATGACAT CACATTTTTA AGTAATGTAA TCTGAGGCCA | |
| | | TTGCTGAGGA AATTAAGAAT TTTCCTTTTT TTTTAACCAC | |
| | | CCCCAGTGAA AAGGATCAGT GTATATTTAT AGCACCTATT | |
| | | TTTTAGTTCT GTCTGTTGTG AGGCACATCC TGCATGGGGC | |
| | | ACTTCTAGTC AAATAGGCAA TGATAAGGAC CTAATTAAAA | |
| | | TGTGATAAGT GTATACTATT ACTTTAAAAG CCTTTACAGT | |
| | | CAGTACTTCA GTTTACAAGG CACTTTCACA GCATCTCGTT | |
| | | TGATCCTCAC AGTCACAACA TGTGGTAGAC AAGGCAGGTG | |
| | | ATTTTTATCC CCATTTTACA GATAAGGAAA CAGGCTGCGG | |
| | | GTGGGGAGTG AGGGGAGGTA AAGATAGTTA GTTGCCTAAG | |
| | | GTCACACAGC CAGTAAGTAA TAGAGCTGGG ACTGGAACCC | |
| | | AGGTTTCCTT ACTCTCATCT ATTGCTCCTC CATATTCCTC | |
| | | ACTCAACCAT GAAAACATTA CTTGAAAGGA CTGATGAGGT | |
| | | TAACCAGAGA CCTAACTGAT ATTGTAACTT TCTATTTTAA | |
| | | GGAAGAATTG TGTCTGTATT TGAGTTCTTT GGAGCCTCCA | |
| | | GTCTGCCTGT GTGTTAGACC AGCACAGCAG TGCTGTGTGA | |
| | | TGCAGCCTGA CCTGTGGCAG GAAAGTAGTG CTTCTGTTTG | |
| | | GAAGTCATGT TCTTTTGCAG CCACACAGGA TCCAAATATC | |
| | | AGTACTATTC CTGTAGTCAA TCTGGGGTCA CATTATAGGT | |
| | | GCCTTATTTC CCTAAGGGTA ACTGATCTGA ATATCTGCAA | |
| | | ATAGGATGAA TCTATTTTTC AGAAGTTCCA TCTTTCATTT | |
| | | TTCTTTTTTT TTTTGAGACA GAGTCTCATT CTGTCGCCCA | |
| | | TGCTGGAGTG CAGTGGCGCG ATCTCGGCTC GCTGCAACCT | |
| | | CTGCCTCCCA GGTTGAAGCA ATTCTCATGC CTCAGCCACC | |
| | | CGAGTAGCTG GGATTACAGG CATGCGCCAT CATGCCCAGC | |
| | | TAATTTATGT ATTTTTAGTA GAGTTGGAGT TTCACCATGT | |
| | | TGGCCAGGCT GGTCTTGGAC TCCTGACCTC AGGTCATCCA | |
| | | CCCGCCTCAG CCTCCCAAAG TGCTGGTATT ACAGGCGTGA | |
| | | GCCACCGCAC CCAGCCCCAT CTTTCATTTT CAAAGAGAAG | |
| | | GGCATTCTAA TAGGAACTGG TGCCAAGAGA GAAGAAAAGA | |
| | | AGTGATAACA GAAGAAATGG CTAGTTACAA TATTAAAAAG | |
| | | CTCCTCTTTG AGATCTCCTC TGCAGGAATA TCAGAGACGG | |
| | | AGTTGAAGCG CTGGAGAGGT AATAGGTCTA GACAGTACAG | |
| | | AACAATAACT GGGGAGTGTG TGAGGATAGA CTGGGCTCCC | |
| | | CCTTGCTTGA AAGATCTCTG GCATTTAATT CTCAATTCTT | |
| | | GATTACTATT TTCCAGTGTA AAACTAGCAC ATATGATCTG | |
| | | ACTACAGGAC AGAGAATTTT AAGTGAAACA TTTGCCTTAC | |
| | | TTGCAGTAAT AATGTGCTGT TCTTCACAGT AGCTAAGGCC | |
| | | CTCTATGTTT CCCAGAGGTA AATAAGAATC CAGGAATGGA | |
| | | GGTCCATCTG TGATGAATGG CTTTTTTCTA ATCAAAGTAG | |
| | | TATAATGCTG TTTTATCTGT TTTGTCATCT TGTTTTTTTT | |
| | | TTTTTTTAAA AAAACAAAAC CTTAATTATA ATATAGCGCA | |
| | | AAGAAAGGCC AGGACTGATG CAGGGATTCC TTGGAAATAT | |
| | | CAGTTCCTAT CACTTTTAAA ACCTGATTTT GGATCTCTCT | |
| | | GTTCTATGTA TGTCTTTAGT GAGAGCACAA TACATGGCAG | |
| | | AACGCTGTGC CAAATGTTAT AGGTAAGGAA TATAGAAATG | |
| | | AATGTTTTTT GTTGTGAAGG TGTTTTCATG TGATATTTTA | |
| | | TAAACACATT TTAAAAAATC TCCATCACTT TTTAGTATAG | |
| | | GAAGGATAGC TTTGCCTGGG AAAAACAGTT TCAACACACC | |
| | | TGCTCAGAGT AGCAGTTCTC CCTCAAAAAA GCAGTGTTCA | |
| | | GCCTGCACTG ACTGTTCTGC TTGCCAAAAG GAGGAAGCAT | |
| | | GCAAGATACT TATTTCTCCA TAGATTGTGG AGTATAGAGG | |
| | | GATGTGGGAC TACAGATTAT TATTTTTTTT CCCCGAGACA | |
| | | GAGTCTTGCT CTGTCGCCCA GGTTGGAACA CAATGGCACG | |
| | | ACCTCAGCTC ACTGCAACCT CTGTCTCCCG GGTTCAAGCA | |
| | | ATTCTCCTGC TTCAGCCTCC TGAGTAGCTG GGATTACAGG | |
| | | CACACACCAC CACCGCACTC AGCTAATTTT TGTATTTTTA | |
| | | GTAGAGGTGG GGTTTTACCA TGTTGGCCAG GCTGGTCTTA | |
| | | AACTCCTGAC CTTGTAATCA TCCCGCCTCG GCCTCCTAAA | |
| | | GTGCTAGGAT TACAGGCATG AGCCACCGCA CCCGGCCCAG | |
| | | ATAATTTTTA ATAGCCTTTG ATCATGGGGT GAGTGAGGGA | |
| | | GTAGGTATAC TTGGCAAATG CATGGTTCTC TGATTTCTAG | |
| | | CTCTAAAGCA GCCTTATCTG AATCCCCAAA TCTTGTGATG | |
| | | CTGAGTACCA TTACTGAACC AGTCTGCACG GTAGGCATCT | |
| | | GCTACCAAAA TTTACCTCCT ACCTGGTAGG TGTCATCTGA | |
| | | TAAGAAAGAA GACAGGTTAT TTTAATTTTT TGAGATAATC | |
| | | ACAGAAAATT GCAGCCCATA CTCTTTATTA CCGAATTCAA | |
| | | GTTTGGAAAT AGACCCTTTG TTTTAAATCA TGATGGGTCT | |
| | | TTATCCCAAT CATTTATCTG GGTCATTTTT CCAACTTTGG | |
| | | AGTTCTAGGA AAGAACCTTG AAAACCTGAT ATGATTCTGC | |
| | | AGCATGAGGT CTACGGTGAC CATTTGGGCA AAGCTCCAGT | |
| | | GGCAATCATT TATTGTGTTT TGCATTTCCT GGGATTTATT | |
| | | GAAATAAGAA TTCACTGTGA TTATGTAGTC TTCTGGCTAG | |
| | | TATCAGGCAG CTCTGCTTTT AATTTGGTTA ATTTTATTTT | |
| | | CTCTGAAGAG GGAGAAGAGG TACAATTTAA TCTTGGCCTC | |
| | | CACAAGCATA TTAAAGCTCA CGTGTTAATC AGTGCATTCT | |
| | | TATGCTCCTA CATTAAATGC CTTGGGTAAA TGGATAAATG | |
| | | GACATGTGCC CAGCTTTAAT TTTTTTTGCA ACAGAAAGAT | |
| | | CAGACTTCCG TATGGCATCG TTGGATTTCA GAGGCTTTCT | |
| | | GGTGTATCTG TAAATCTGAA TGTTGCCTTC TGCCAGTCTG | |
| | | TATAACCAGG TGATTCATGC TGCAAATGAA ATCAGGAAGC | |
| | | AGTAAAGTGT TAAAGCAAGA GTATTGTCCA ATTCACTTGT | |
| | | CTTCCTGATC CTTGTACTTT ATTTCACGTG TCGGTGTTTA | |
| | | CATTACATAC TTATATTTCC TGTGAAAGAA AGAGTTAAAT | |
| | | AAATTGTAGC AGTTTGA | |
| SEPN | NM_031475.2 | AGCGGAGCGC CAGGCAGCGC GGAGCGGAGG CCAGGCCCAC | 29 |
| | | AGCCGCTCCG CCTCCCGGCC CGCAGATCCC CGACGGCCGC | |
| | | ACCGCGGGCT CCTCTGGCCC GCAAGAACAC GTGCATGGCG | |
| | | TCCTGGGGAA GGCGCTGAGT GCGGAGTCGC GGCGCCGCAC | |
| | | GCGGCACCAT GGCCCTGGAG CAGGCGCTGC AGGCGGCGCG | |
| | | GCAGGGCGAG CTGGACGTGC TGAGGTCGCT GCACGCCGCA | |
| | | GGCCTCCTGG GGCCCTCGCT GCGCGACCCG CTGGACGCGC | |
| | | TGCCCGTGCA CCACGCGGCC CGCGCTGGGA AGCTGCACTG | |
| | | TCTGCGCTTC CTGGTGGAGG AAGCCGCCCT CCCCGCCGCG | |
| | | GCCCGCGCCC GCAACGGCGC CACACCGGCC CACGACGCCT | |
| | | CCGCCACCGG CCACCTCGCC TGCCTGCAGT GGCTGCTGTC | |
| | | GCAGGGCGGC TGCAGAGTGC AGGACAAAGA CAATTCTGGT | |
| | | GCCACAGTCT TGCATCTGGC TGCCCGCTTC GGCCACCCCG | |
| | | AGGTGGTGAA CTGGCTCTTG CATCATGGCG GTGGGGACCC | |
| | | CACCGCGGCC ACAGACATGG GCGCCCTGCC TATCCACTAC | |
| | | GCTGCCGCCA AAGGAGACTT CCCCTCCCTG AGGCTTCTCG | |
| | | TCGAGCACTA CCCTGAGGGA GTGAATGCCC AAACCAAGAA | |
| | | CGGTGCCACG CCCCTGTACC TGGCGTGCCA GGAGGGCCAC | |
| | | CTGGAGGTGA CCCAGTACCT GGTGCAGGAA TGCGGCGCAG | |
| | | ACCCGCACGC GCGCGCCCAC GACGGCATGA CCCCGCTGCA | |
| | | CGCCGCGGCG CAGATGGGCC ACAGCCCAGT CATCGTGTGG | |
| | | TTGGTGAGCT GCACCGACGT GAGCCTGTCC GAGCAGGACA | |
| | | AAGACGGCGC CACCGCCATG CACTTCGCGG CGAGCCGCGG | |
| | | CCACACCAAG GTGCTCAGCT GGCTGCTGCT GCACGGCGGG | |
| | | GAGATCTCGG CTGACCTGTG GGGCGGGACC CCGCTGCACG | |
| | | ACGCCGCCGA GAACGGGGAG CTAGAGTGCT GCCAGATCCT | |
| | | GGTAGTGAAC GGCGCGGAGC TGGACGTCCG CGACCGCGAC | |
| | | GGGTACACGG CCGCCGACCT GTCGGACTTC AACGGCCACA | |
| | | GCCACTGCAC CCGCTACCTG CGCACGGTGG AGAACCTGAG | |
| | | CGTGGAGCAC CGCGTGCTTT CCCGGGATCC ATCCGCAGAG | |
| | | CTGGAGGCTA AGCAGCCGGA TTCAGGCATG TCCTCACCCA | |
| | | ATACCACGGT GTCGGTCCAG CCGCTGAACT TTGACCTCAG | |
| | | CTCGCCTACC AGCACCCTCT CCAACTACGA CTCCTGCTCC | |
| | | TCCAGCCACT CCAGCATCAA GGGCCAGCAC CCTCCATGTG | |
| | | GGCTTTCCAG CGCTAGAGCT GCAGACATAC AGAGCTACAT | |
| | | GGACATGCTG AACCCGGAGC TGGGCCTGCC TCGGGGCACG | |
| | | ATTGGGAAGC CCACACCCCC ACCACCCCCA CCCAGCTTCC | |
| | | CCCCGCCACC CCCGCCCCCA GGCACCCAAC TGCCCCCACC | |
| | | CCCACCTGGC TACCCAGCTC CCAAGCCTCC TGTAGGACCA | |
| | | CAGGCAGCTG ACATCTACAT GCAGACCAAG AACAAACTCC | |
| | | GCCACGTGGA GACAGAGGCC CTCAAGAAGG AGCTGAGCTC | |
| | | CTGTGACGGC CACGACGGGC TGCGGAGGCA GGACTCCAGC | |
| | | CGCAAGCCCC GCGCCTTCAG CAAGCAGCCC AGCACGGGGG | |
| | | ACTACTACCG GCAGCTGGGC CGCTGCCCCG GCGAGACGCT | |
| | | GGCCGCACGC CCGGGCATGG CGCACAGCGA GGAGGTGCGT | |
| | | GCCCGCCAGC CCGCGCGCGC CGGCTGCCCG CGCCTCGGCC | |
| | | CTGCCGCCCG CGGCTCACTC GAAGGCCCCT CCGCTCCCCC | |
| | | GCAGGCGGCG CTGCTTCCTG GGAACCATGT TCCTAACGGC | |
| | | TGCGCCGCGG ACCCCAAGGC GTCCAGGGAG CTGCCACCGC | |
| | | CGCCCCCACC GCCGCCGCCG CCCCTGCCGG AGGCCGCGAG | |
| | | TTCGCCACCG CCGGCCCCGC CTCTGCCCCT CGAGAGCGCT | |
| | | GGCCCTGGCT GCGGGCAGCG CCGCTCCTCC TCGTCCACCG | |
| | | GCAGCACCAA GTCTTTCAAC ATGATGTCCC CGACGGGCGA | |
| | | CAACTCGGAG CTACTGGCTG AGATTAAGGC AGGCAAGAGC | |
| | | CTGAAGCCGA CGCCCCAGAG CAAGGGGCTG ACCACAGTGT | |
| | | TCTCAGGCAT CGGGCAGCCG GCCTTCCAGC CCGATTCGCC | |
| | | GCTGCCTTCT GTGTCACCTG CACTGTCACC AGTCCGGAGC | |
| | | CCCACACCGC CAGCTGCGGG GTTTCAGCCG CTGCTCAATG | |
| | | GAAGCTTGGT TCCCGTGCCG CCCACTACTC CTGCGCCGGG | |
| | | AGTGCAGCTG GACGTGGAGG CTCTCATCCC CACGCACGAT | |
| | | GAGCAGGGCC GGCCCATCCC CGAGTGGAAG CGCCAGGTGA | |
| | | TGGTGCGCAA GATGCAGCTG AAGATGCAGG AGGAGGAGGA | |
| | | GCAGAGGCGG AAGGAGGAGG AGGAGGAGGC CCGGCTGGCC | |
| | | AGCATGCCCG CCTGGAGGCG GGACCTCCTG CGGAAGAAGC | |
| | | TGGAAGAAGA GAGGGAGCAG AAGCGGAAAG AGGAGGAGCG | |
| | | ACAGAAGCAG GAGGAGCTGC GGCGGGAGAA GGAACAGTCA | |
| | | GAGAAGCTGC GGACGCTGGG CTACGATGAG AGCAAGCTGG | |
| | | CGCCCTGGCA GCGACAGGTC ATCCTGAAGA AGGGGGACAT | |
| | | CGCTAAGTAC TAGAGGCCGC AGACTCCTGT CCGCAGCCTC | |
| | | GCAGCTCCGT GGGGCCCTCC GCCCCAGCCC CAGCCAGCCA | |
| | | GGCCCTGGTG GAAAGGCTGG GAGCCGCACA GCCCTCCCCT | |
| | | CCTGCGCTGG AAACCCTCCC TGACCCCCAC CCTGGCCCCC | |
| | | CGTATCCCCA GCCCTTGGCA ACACTGGAGT GCACACGCCG | |
| | | CCACGGTTGC CCAGAAAAAG TGCCCAAGCT GCTGACGCAA | |
| | | ACAACAACAA ATGCTGCTTA TTTGCATGCC GACTTACATA | |
| | | TATTTGCATG TTCGTTGACT ATCAAAGAGT GCAGAGCTCT | |
| | | CCCCAGCCCC GTGGGTGGTG ACTTTGTTTT CCTGCGGGGC | |
| | | TCAGCCCCCT CCAGGATGCA GCCCCCTCCC CCGCACCCCG | |
| | | GAACCGGCGT CGCTGGCGCA TCCTGGGTGG AGGCAGGCCC | |
| | | CGAGCTCGGG GAAGGGGTTT TCCCTTCCTC TCTGACCCAG | |
| | | ATCTGCGCGC GGCCTAGCCC GGGCCTCATT TCTTATCCCC | |
| | | GCCAAGGGTT TCCTCTCAGT CATTTGTTTA CCAGAAACAT | |
| | | GAAAACTGCC TGTCTGGCCG GGCCGCACTT GTGGCCCCCG | |
| | | GGACCCCACC TCTGGCCCCA CCTCCCTCAA GTCTGCGCCC | |
| | | CGTCCCCAGC CAGACCCACT CGCTGCCGGG ACCCTTTCAC | |
| | | TGCCCCGGTG GAGTGAATAG AGGATGAGGG GCCCTGACCC | |
| | | TGTGTCTCCA ACTGCTGCAC CCCATCCCGA CCCTGTCTCC | |
| | | GCCACCTCGC AGCCCCATTA AAGCGCTCTC ATCTGGGCTC | |
| | | CGGTTCACTC A | |
| YWHAQ | NM_006826.3 | TTGGGCGGTG GACCGCCCCT CGGCCCCGGG GTAGGCTGAC | 30 |
| | | ACGGGAGGGT CCTCAGCTAA AGCCAAAAGC AGATCAAAGT | |
| | | GGTGGGACTC GCGTCGCGGC CGCGGAGACG TGAAGCTCTC | |
| | | GAGGCTCCTC CCGCTGCGGG TCGGCGCTCG CCCTCGCTCT | |
| | | CCTCGCCCTC CGCCCCGGCC CCGGCCCCGC GCCCGCCATG | |
| | | GAGAAGACTG AGCTGATCCA GAAGGCCAAG CTGGCCGAGC | |
| | | AGGCCGAGCG CTACGACGAC ATGGCCACCT GCATGAAGGC | |
| | | AGTGACCGAG CAGGGCGCCG AGCTGTCCAA CGAGGAGCGC | |
| | | AACCTGCTCT CCGTGGCCTA CAAGAACGTG GTCGGGGGCC | |
| | | GCAGGTCCGC CTGGAGGGTC ATCTCTAGCA TCGAGCAGAA | |
| | | GACCGACACC TCCGACAAGA AGTTGCAGCT GATTAAGGAC | |
| | | TATCGGGAGA AAGTGGAGTC CGAGCTGAGA TCCATCTGCA | |
| | | CCACGGTGCT GGAATTGTTG GATAAATATT TAATAGCCAA | |
| | | TGCAACTAAT CCAGAGAGTA AGGTCTTCTA TCTGAAAATG | |
| | | AAGGGTGATT ACTTCCGGTA CCTTGCTGAA GTTGCGTGTG | |
| | | GTGATGATCG AAAACAAACG ATAGATAATT CCCAAGGAGC | |
| | | TTACCAAGAG GCATTTGATA TAAGCAAGAA AGAGATGCAA | |
| | | CCCACACACC CAATCCGCCT GGGGCTTGCT CTTAACTTTT | |
| | | CTGTATTTTA CTATGAGATT CTTAATAACC CAGAGCTTGC | |
| | | CTGCACGCTG GCTAAAACGG CTTTTGATGA GGCCATTGCT | |
| | | GAACTTGATA CACTGAATGA AGACTCATAC AAAGACAGCA | |
| | | CCCTCATCAT GCAGTTGCTT AGAGACAACC TAACACTTTG | |
| | | GACATCAGAC AGTGCAGGAG AAGAATGTGA TGCGGCAGAA | |
| | | GGGGCTGAAA ACTAAATCCA TACAGGGTGT CATCCTTCTT | |
| | | TCCTTCAAGA AACCTTTTTA CACATCTCCA TTCCTTATTC | |
| | | CACTTGGATT TCCTATAGCA AAGAAACCCA TTCATGTGTA | |
| | | TGGAATCAAC TGTTTATAGT CTTTTCACAC TGCAGCTTTG | |
| | | GGAAAACTTC ATTCCTTGAT TTGTGTTTGT CTTGGCCTTC | |
| | | CTGGTGTGCA GTACTGCTGT AGAAAAGTAT TAATAGCTTC | |
| | | ATTTCATATA AACATAAGTA ACTCCCAAAC ACTTATGTAG | |
| | | AGGACTAAAA ATGTATCTGG TATTTAAGTA ATCTGAACCA | |
| | | GTTCTGCAAG TGACTGTGTT TTGTATTACT GTGAAAATAA | |
| | | GAAAATGTAG TTAATTACAA TTTAAAGAGT ATTCCACATA | |
| | | ACTTCTTAAT TTCTACATTC CCTCCCTTAC TCTTCGGGGG | |
| | | TTTCCTTTCA GTAAGCAACT TTTCCATGCT CTTAATGTAT | |
| | | TCCTTTTTAG TAGGAATCCG GAAGTATTAG ATTGAATGGA | |
| | | AAAGCACTTG CCATCTCTGT CTAGGGGTCA CAAATTGAAA | |
| | | TGGCTCCTGT ATCACATACG GAGGTCTTGT GTATCTGTGG | |
| | | CAACAGGGAG TTTCCTTATT CACTCTTTAT TTGCTGCTGT | |
| | | TTAAGTTGCC AACCTCCCCT CCCAATAAAA ATTCACTTAC | |
| | | ACCTCCTGCC TTTGTAGTTC TGGTATTCAC TTTACTATGT | |
| | | GATAGAAGTA GCATGTTGCT GCCAGAATAC AAGCATTGCT | |
| | | TTTGGCAAAT TAAAGTGCAT GTCATTTCTT AATACACTAG | |
| | | AAAGGGGAAA TAAATTAAAG TACACAAGTC CAAGTCTAAA | |
| | | ACTTTAGTAC TTTTCCATGC AGATTTGTGC ACATGTGAGA | |
| | | GGGTGTCCAG TTTGTCTAGT GATTGTTATT TAGAGAGTTG | |
| | | GACCACTATT GTGTGTTGCT AATCATTGAC TGTAGTCCCA | |
| | | AAAAAGCCTT GTGAAAATGT TATGCCCTAT GTAACAGCAG | |
| | | AGTAACATAA AATAAAAGTA CATTTTATAA ACCATTTACT | |
| | | ATGGCTTTGT AACAATTGCA TACCCATATT TTAAGGGACA | |
| | | GGTGAATTTA CTACTTTCTA AAGTTTATTG ATACTTCCCT | |
| | | TTTATGTAAA ATGTAGTAGT GATACCTATA TTTCCACATT | |
| | | GTGCATTGTG ACACACTTGT CTAGGGATGC CTGGAAGTGT | |
| | | ATAAAATTGG ACTGCATTTC TTAGAGTGTT TTACTATAGA | |
| | | TCAGTCTCAT GGGCCATCTC TTCCTCAGAT GTAAATGATA | |
| | | TCTGGTTAAG TGTTATATGG AATAAAGTGG ACATTTTAAA | |
| | | ACTAGCAAAG TTAAAAAAAA AAAAAAAAAA AA | |
| VPS37A | NM_001145152.1 | GCAGAGGGGG CGGAGAGCGC CCCCGGGGGC GGGGCACGCA | 31 |
| | | AGTGACGGCG GCGCGGGTGG TGGAGCGCTG GGCGGCCAGG | |
| | | CTCCCTGGCT GGCCGGTTTG GGCGTCTGGG CCGTGAAGGT | |
| | | GGGACCTCCT GTTCCGGGCC GCAAGTTTCC CTCTCCAGCC | |
| | | GCCCGCCGTT CGTAGCATGT CCCCCAGAAC TCGGGGAGCG | |
| | | CAGGCAGGAC AGGCTTAGAG AAGACGCGGT CCCCAGCGCT | |
| | | TGGGCCACGG ACGTCCCACC CCGCTCCTCT GTCGCTGGAG | |
| | | AACCGCCGGG CCGAGCCACT GGGAGAAGCA GGCCAGAGCC | |
| | | TTCCAGGGCC TCCGGCCCGT GGACCCGAGG AGGATGAGCT | |
| | | GGCTTTTTCC CCTGACCAAG AGCGCCTCCT CCTCCGCGGC | |
| | | TGGGTCCCCC GGTGGCCTCA CCAGCCTCCA GCAGCAGAAG | |
| | | CAGCGCCTGA TCGAGTCCCT CCGGAACTCA CACTCCAGAT | |
| | | TGCTTCCTCC ACAGTTTCCT CAGGAAAAAC CAGTGATCAG | |
| | | TGTTTATCCA CCAATACGAC ATCACTTAAT GGATAAACAA | |
| | | GGAGTGTATG TTACCTCTCC ATTAGTAAAC AATTTTACAA | |
| | | TGCACTCAGA TCTTGGAAAA ATTATTCAGA GTCTGTTGGA | |
| | | TGAGTTTTGG AAGAATCCTC CAGTTTTAGC TCCTACTTCA | |
| | | ACAGCATTTC CTTATCTATA CAGTAACCCA AGTGGGATGT | |
| | | CTCCTTATGC TTCTCAGGGT TTTCCATTTC TTCCTCCATA | |
| | | TCCTCCACAA GAAGCAAACA GGAGTATCAC TTCTTTATCT | |
| | | GTTGCTGACA CTGTTTCTTC TTCAACAACA AGTCATACCA | |
| | | CAGCCAAGCC TGCCGCTCCT TCATTTGGTG TCCTTTCAAA | |
| | | TCTGCCATTA CCCATTCCCA CAGTGGATGC TTCAATACCG | |
| | | ACAAGCCAAA ATGGTTTTGG GTACAAGATG CCAGATGTCC | |
| | | CTGATGCATT TCCAGAACTC TCAGAACTAA GTGTGTCACA | |
| | | ACTCACAGAT ATGAATGAAC AAGAGGAGGT ATTACTAGAA | |
| | | CAGTTTCTGA CTTTGCCTCA ACTAAAACAA ATTATTACCG | |
| | | ACAAAGATGA CTTAGTAAAA AGTATTGAGG AACTAGCAAG | |
| | | AAAAAATCTC CTTTTGGAGC CCAGCTTGGA AGCCAAAAGA | |
| | | CAAACTGTTT TAGATAAGTA TGAATTACTT ACACAGATGA | |
| | | AGTCCACTTT CGAAAAGAAG ATGCAAAGGC AGCATGAACT | |
| | | TAGTGAGAGC TGTAGTGCAA GTGCCCTTCA GGCAAGATTG | |
| | | AAAGTAGCTG CACATGAAGC TGAGGAAGAA TCTGATAATA | |
| | | TTGCAGAAGA CTTCTTGGAG GGAAAGATGG AAATAGATGA | |
| | | TTTTCTCAGT AGCTTCATGG AAAAGAGAAC AATTTGCCAC | |
| | | TGTAGAAGAG CCAAGGAAGA GAAACTTCAG CAGGCGATAG | |
| | | CAATGCACAG CCAATTTCAT GCTCCACTAT AGATTTTCCT | |
| | | GGAAACATGA ACTGCCAAGA GAGGAATGGG ACACAAAACC | |
| | | AAACACTGTT TTATATTTAT GGTTTGCAAA CTGGCATTTC | |
| | | ATCAGTGGCT AAATTCACAG ATATCCTATA TAGATTGTAT | |
| | | ACAGAACTGA GACTGATTTT GTACCGATTA GAATGATTGC | |
| | | TATGATCTTT GAGAAATTTT TCTGCACTAT TTGCACTGAA | |
| | | ATGTTTATTT ATTGTTGATA AATTGTATCA TATTTAAGTT | |
| | | CCACTGCTGT TCCTCTTACC TTGATTAAAT GCCTATGCAT | |
| | | GTACTTTTAG CTAGTTTTTA ATATTTTATA AAACTTCATT | |
| | | TAAATTTGTA TTTTTAACTT GAAGTTCCAT TTCTTTATCA | |
| | | AGGATGGTAT TTAGATTTTT TTCCTCTTAA CCTTTTTTCA | |
| | | AAAACTATTT TCAACTGTGA GGAAACCCTT ATTTTTCTTT | |
| | | CTTTGTGGAT AAAACTTTCA AAAGCAATTT AAGATATTCA | |
| | | TAGTGTTAGG AAACACCAAA CCTGCCTATG TGCCATCTCA | |
| | | CAAAAGAAAC TTTTAATACC TACAATAAAT CAAAAGAATA | |
| | | AACCAGCTGT TCTTATATAT TGTTTCATTT TTAAAACTAA | |
| | | AGATGCATTT AAGAAGCAAT ACAAGTAAAT ATTTTACCTA | |
| | | ATAGGAAAAA AAAAAGTTGC CTTTCATTTA AACCATTCCA | |
| | | ACAGAAATTC TTATGCTAAT TTAAAACATA TATATATCTG | |
| | | GTAGGTTTGT GGTTGGATAG GTTTTCTAAA TTCCTAATGT | |
| | | TAAAAACAAT CTTTATGTTA ATATACACTA AATCTATACA | |
| | | CAAAAAAAGT CAGTGAACTT TTCTGACCTT TACTGTGAGT | |
| | | TACCTTTTCC TAAGAGGAAA GCTATAGTAA TAAGTAAAAT | |
| | | TTAATTTTTA GGCAATCCTG ATTTTTAATG AATTTAATTG | |
| | | AGTGTTCTTG TATACTACAT TGAGCAGTTT GCTTCTATAC | |
| | | CGTGTCACAA AATTCATGTA TTTCTTGAGA AGCCCTAAAA | |
| | | GCTCATAAAG GAAAATGCCG TGAACTATGT AGCTCAGGCT | |
| | | TGGTAAGGTG CCATCTAAAT TACAAAACAA ACTAATGCAT | |
| | | AATTTTGCTT AAATTTCATC CCAGTATGAT TGTCTTCCCA | |
| | | ACACCAGCAT ATAGTATAGA TTGTCTGTCT TTTTTATATT | |
| | | TTTTAGTTCT TCCTGTACAT GTTTTTGGCA ATAAAGTTAT | |
| | | AGGAAGAACA AAATTATTTT GTTAGAATTA AAACATGCTT | |
| | | AATATTTAGT CTGTTTGTGG AGGGCAGGTA TTCACGTGGA | |
| | | CTGAGATACA ATGTTGGATA CAGAAAATAA CTTTCATTGT | |
| | | CTTCCTGACA CTGTGCTAAG GACATGCTGT TAAAGCTTCA | |
| | | AAGTGACCAG ATGAGGAAGG AATAATTAAT TATTACTCCT | |
| | | GATTTGTAGA TAACTGAGGT AAGAGTGTTT CAAATTTATG | |
| | | ATAGTCTTTT GGGTATTCAG AAACCTTTCC TTATACTGCA | |
| | | CTGGCCACCA GAGCTTAATT TTCCCAGCAG TTACAGCAAT | |
| | | GGGAGATAGA ACAGTCTCAA TCTTTTGCCA ACCATCAGGT | |
| | | TCCTAGAAAC CAGGTAGGTG TATCCCATAA CAAGGGAGGA | |
| | | GCATACCACA GCCCCTCATT TGATTAATTC ATTTGATCTA | |
| | | TCTATGTTAT TAAGTACCTA CTAGGAATAA GGCATTGTGG | |
| | | AAATACTATA CAAAGATAAA CATTGTTTAG ATGCTTATCT | |
| | | ACTTTCCTTT TCACCAGAAA AACAGAAAAA AAAGAAACAT | |
| | | TTTCTTACAG AGTAAAAATG TTCTACATAA TCACATGAGT | |
| | | AGTTCATCTC AGTGTTTTTT ATTCTTTAAA GTTGAACTAT | |
| | | CCCAGTTTCA TTCTATACCA TTCATTGGAT AACCTTGTTA | |
| | | CAACCCAGTC ATGAAACAGA GCAGTGTGAT CAGTTATCTG | |
| | | CATTTAACAA ATAGACAAAT CAGTTTACAT AAAGGTTATG | |
| | | TATGTCACCC ACGATGAAAA GAATCTGCAT TTGAATATGC | |
| | | CCGTATGAAT GTGGGTTCTG TTTTTGCAAC AGAGATTAAG | |
| | | TGACCATTTT TTCTAATTTT ATGGCTATAT ATTTTCTTCA | |
| | | TAAAAATTGG TCACATCGGA GAAGCAGTGC CACAGGAAAA | |
| | | ATGAAATGCA TGTGAAAGTT TGTATTCTGA TTTTACAAGA | |
| | | TGAGATAGAA ATCAGAATTA AAGAGGAATA CTTAGGAGTT | |
| | | ACTAGGCTAA TCAGTGTACG AATTTGTCAT AGGTAGAGAT | |
| | | TTAAAGGTTA ATATCTTAAA ATAGAAGAAA ATTCTAAATC | |
| | | AATCAATCAG TGAGATATAA ACTAAACAGA CCCACTTCAA | |
| | | AGTTGAAAGA AATTTCTAGG CATAAATTGA GACTAGGAAA | |
| | | TTTATATCAG AATAGAGGGT GCTTGACACA TATATATGCT | |
| | | TAAATTGAAG GACAGCTCAG ATTCATTTTT AGGAGAAGAA | |
| | | AGTAAACTAA TGTGCTCTTA AAGAATAAAA ATTTATTCTA | |
| | | TGGTTTCTGT CTCTGATCAT CACCTTCCAT TCTATAAAAA | |
| | | GCTCAGTTAC TGATTTGCTG GGTCATGGTC AAAATTCTTA | |
| | | CCTATTTATT TCATATCAAC TTTAAAAAAT AAATTACTTG | |
| | | CATTCTATAT ATTACTAATT GGGAAGTAAT ATGCCTCAAA | |
| | | TCAGTTTTAT ACTGGATTAT TCCCTATGCT TTAAACCACT | |
| | | GCTCTCAATA AAACACTTCC TGATTAATGT TTGATTATTA | |
| | | GATATTTTAG TCTTGTTGGG GATATTTTAG TCTTGTTGGG | |
| | | TTAGCCATGC TCTGAAGAAT CTGTGAAAGT ACAGTAAAGT | |
| | | TTTAATAAGC AATAAATGTA ACCTTTTATA TAAATCTCAG | |
| | | TGCTAGGTTA ACTTCTAATA AGCAGACGAA CATGTTACAT | |
| | | AAATTATAAT GTCTGTCTTG TAAAAAAGTT GAGGGGACTA | |
| | | AAAGTTTATG ACTCTGATAT GGAAGTTGTC ATATTAAAAA | |
| | | ACTACATTTT AAAACATCAA ATATTTATAC TATTTGCTTT | |
| | | TCAAATAAAA GCATAGTGCT GTTTGGCATA | |
| PRRC2B | NM_013318.3 | GCAGATCGGG AGCGGTGCCG AGAAAAATTT CCTTACTAGA | 32 |
| | | TGACATTTCA TCGCAATGTC CGATCGTTTG GGGCAAATTA | |
| | | CCAAGGGCAA GGATGGGAAA AGCAAGTACT CGACTCTCAG | |
| | | CCTGTTTGAT AAGTATAAAG GAAAATCAGT AGACGCGATT | |
| | | AGATCCTCAG TTATTCCTAG ACATGGCTTA CAGAGTCTTG | |
| | | GGAAAGTTGC TGCAGCCCGG CGCATGCCAC CGCCTGCAAA | |
| | | CCTGCCAAGC TTGAAGTCTG AAAACAAAGG AAACGACCCC | |
| | | AACATCGTGA TAGTACCCAA GGACGGGACG GGATGGGCAA | |
| | | ACAAGCAGGA TCAGCAAGAC CCAAAGAGTT CCAGTGCGAC | |
| | | GGCCTCTCAG CCGCCGGAGT CGCTGCCGCA GCCGGGTTTG | |
| | | CAGAAATCTG TCTCCAATTT GCAGAAACCG ACACAGTCAA | |
| | | TCAGTCAGGA GAATACAAAT TCAGTGCCAG GTGGACCAAA | |
| | | GTCATGGGCA CAGCTGAATG GAAAGCCAGT AGGACACGAA | |
| | | GGTGGTTTAA GGGGCTCAAG CCGACTGTTA TCCTTCTCTC | |
| | | CCGAGGAATT TCCGACGCTG AAAGCAGCTG GAGGGCAGGA | |
| | | CAAGGCTGGC AAAGAAAAGG GCGTCTTAGA TCTGTCGTAT | |
| | | GGGCCAGGAC CAAGCCTCCG CCCTCAGAAT GTGACAAGCT | |
| | | GGAGGGAGGG CGGTGGGCGA CACATAATTT CTGCCACGTC | |
| | | TCTGAGCACC TCCCCAACTG AGCTGGGCAG CAGGAACTCG | |
| | | AGTACGGGAG ATGGAGCCCC CTCCTCGGCA TGTACCAGCG | |
| | | ATTCTAAGGA CCCCTCTCTC CGCCCGGCTC AGCCTGTCCG | |
| | | AAAAGGGGCT TCACAGTTCA TGGGAAATGT ATACCACCCA | |
| | | CCTACATACC ATGACATGCT TCCTGCTTTT ATGTGTTCGC | |
| | | CGAAGTCATC AGAAAACCAG GGTACAGTGG AACGAGGCTC | |
| | | TTTTCCCCTT CCTCAGCTCC GCCTTGAACC TCGAGTTCCT | |
| | | TTTAGACAGT TCCAGATGAA TGACCAAGAC GGAAAAGAAA | |
| | | ACAGGCTGGG ATTGTCTCGC CCACTCCGCC CACTAAGGCA | |
| | | GCTGGTGGAG CGGGCACCAC GGCCCACCAT TATCAATGCG | |
| | | GAAAACCTGA AGGGCCTTGA CGATCTGGAC GCCGATGCCG | |
| | | ATGATGGCTG GGCAGGCCTC CATGAAGAAG TGGACTATTC | |
| | | TGAGAAACTG AAGTTCAGTG ATGATGAAGA GGAGGAAGAA | |
| | | GTTGTGAAGG ACGGCAGGCC AAAGTGGAAC AGTTGGGACC | |
| | | CTAGGAGGCA GCGGCAGTTG TCAATGAGCT CTGCAGACAG | |
| | | TGCGGACGCT AAGCGGACTC GAGAGGAAGG GAAGGACTGG | |
| | | GCTGAAGCAG TGGGTGCGTC CCGTGTGGTC CGAAAGGCGC | |
| | | CAGACCCTCA GCCACCGCCC AGGAAGCTTC ATGGCTGGGC | |
| | | ACCAGGCCCT GACTACCAGA AGTCATCAAT GGGCAGCATG | |
| | | TTCCGGCAAC AGTCCATCGA GGACAAGGAG GACAAGCCCC | |
| | | CACCAAGGCA GAAGTTCATT CAGTCAGAGA TGTCCGAGGC | |
| | | GGTGGAGCGA GCCCGAAAGC GCCGGGAAGA AGAGGAGCGC | |
| | | CGAGCCCGGG AGGAGAGGCT GGCCGCCTGT GCTGCCAAAC | |
| | | TCAAGCAGCT GGACCAGAAG TGTAAGCAGG CACGAAAGGC | |
| | | AGGTGAGGCC CGGAAGCAGG CAGAGAAGGA AGTGCCCTGG | |
| | | TCTCCAAGTG CTGAGAAGGC ATCTCCCCAG GAAAACGGCC | |
| | | CTGCTGTCCA CAAAGGCTCC CCAGAATTCC CTGCCCAAGA | |
| | | GACCCCCACC ACATTCCCAG AAGAGGCACC CACAGTGTCC | |
| | | CCAGCAGTGG CACAGAGCAA CAGCAGTGAG GAAGAGGCCA | |
| | | GAGAGGCTGG GTCCCCTGCA CAGGAGTTCA AGTATCAGAA | |
| | | GTCCCTTCCT CCCCGATTCC AGCGCCAGCA GCAGCAACAA | |
| | | CAGCAGGAGC AGCTGTACAA GATGCAGCAC TGGCAGCCGG | |
| | | TGTACCCCCC GCCGTCCCAC CCCCAGCGCA CCTTTTACCC | |
| | | ACACCACCCC CAGATGTTGG GCTTCGATCC CAGGTGGATG | |
| | | ATGATGCCTT CCTACATGGA CCCACGTATC ACGCCCACTC | |
| | | GGACCCCGGT GGACTTCTAC CCCTCCGCCC TGCATCCCTC | |
| | | AGGACTGATG AAGCCCATGA TGCCCCAGGA GTCCCTCAAT | |
| | | GGGACAGGCT GTCGCTCTGA GGATCAGAAC TGTGTGCCCC | |
| | | CACTCCAAGA AAGAAAAGTG ACCCCCATCG ACTCACCCCC | |
| | | TGTGTGGAGC CCAGAGGGCT ACATGGCACT GCAGAGCAAG | |
| | | GGCTACCCGC TCCCGCACCC GAAGTCGAGT GACACCTTGG | |
| | | CTATGGACAT GCGTGTCAGG AATGAAAGCT CTTTCTCTGC | |
| | | CTCACTCGGA AGGGCAGGGG GCGTAAGTGC TCAGCGCGAT | |
| | | CTCTTTGAGG AGAGAGGGGA GGAGTACTTG AGTGCTTTTG | |
| | | ACAAGAAGGC CCAAGCAGAC TTTGACAGCT GTATCTCTTC | |
| | | TCAAAGAATA GGCCAGGAGC TTTTGTTTCC ACCCCAAGAA | |
| | | AATGTTCAGG ATGCAGGTGC TCCTGGGGGT CACACCCAAA | |
| | | ACCTCAGGTG TTCCCCATTG GAGCCTGACT TTGTCCCAGA | |
| | | TGAGAAAAAG CCAGAGTGTG GCAGTTGGGA TGTTAGCCAC | |
| | | CAGCCAGAGA CCGCTGACAC AGCCCATGGT GTTGAGCGGG | |
| | | AGACACCCCG GGAGGGGACG GCCTTTAACA TCTCCTCCTG | |
| | | GGACAAGAAC GGGAGCCCCA ACAAACAGCC ATCCTCGGAG | |
| | | CCTGAATGGA CTCCCGAGCC CCGGAGCTCC AGCAGCCAGC | |
| | | ACCCGGAGCA GACGGGCAGG ACCCGGAGGT CGGGACCCAT | |
| | | CAAGAAACCA GTCCTGAAAG CCCTCAAGGT GGAAGACAAG | |
| | | GAGAAGGAGC TTGAGAAGAT TAAGCAGGAG CTAGGGGAGG | |
| | | AGAGTACCCG GCTGGCCAAG GAGAAGGAGC AGAGCCCCAC | |
| | | GGCAGAAAAG GATGAGGACG AAGAGAACGA TGCCTCTCTG | |
| | | GCCAACTCCT CCACCACCAC TTTGGAGGAC AAAGGCCCTG | |
| | | GCCATGCCAC TTTTGGCCGC GAGGCCACCA AATTTGAAGA | |
| | | GGAGGAGAAA CCTGACAAGG CCTGGGAAGC CAGACCCCCA | |
| | | CGAGAGTCCA GCGATGTTCC CCCCATGAAG AGAAATAACT | |
| | | GGATCTTTAT TGATGAGGAG CAAGCCTTTG GGGTCAGAGG | |
| | | ACAGGCCCGG GGCCGGGGCC GTGGTTTCAG AGAGTTCACT | |
| | | TTTCGTGGTC GGCCTGCTGG CGGAAATGGG AGCGGCCTCT | |
| | | GTGGTGGGGG GGTCCTGGGG GCCCGCAGCA TCTACTGCAG | |
| | | CAGTCAGCGC AGCGGCCGTG GCCGGGGCCT GCGAGAGTTT | |
| | | GCGCGGCCAG AGGACTGCCC CAGAGCCAAG CCCCGACGGA | |
| | | GAGTTGCCAG TGAGACCCAT AGCGAGGGCT CAGAGTATGA | |
| | | AGAACTTCCC AAGCGCCGCC GGCAGAGGGG CTCCGAGAAC | |
| | | GGGAATGAAG GCTCGCTCCT GGAGAGGGAG GAGAGCACCT | |
| | | TGAAGAAGGG CGACTGCAGA GATTCTTGGC GGTCCAACAA | |
| | | GGGGTGCTCT GAGGACCACA GCGGTCTAGA TGCCAAGAGC | |
| | | CGAGGCCCTC GGGCCTTTGG GCGAGCCCTC CCTCCCCGGC | |
| | | TGAGCAATTG CGGGTATGGA CGGAGAACCT TCGTCTCCAA | |
| | | AGAGTCACCC CACTGGCAGA GCAAAAGTCC AGGCAGCTCT | |
| | | TGGCAGGAAT ATGGCCCTTC CGACACATGC GGATCCCGGC | |
| | | GACCTACAGA CAGAGACTAT GTCCCAGATT CCTACAGACA | |
| | | CCCTGACGCA TTTGGTGGCC GGGGCTTTGA GGACAGCCGC | |
| | | GCGGAGGACA AGAGATCCTT CTTCCAAGAT GAACACGTGG | |
| | | CAGATTCTGA AAATGCAGAG AACCGGCCCT TCAGGAGAAG | |
| | | GCGCCCCCCA CGCCAAGATA AGCCCCCTCG ATTCCGGCGC | |
| | | CTCCGGCAAG AGCGGGAGTC CCTGGGCCTG TGGGGACCCG | |
| | | AGGAGGAGCC CCACCTGCTG GCAGGTCAGT GGCCAGGCAG | |
| | | GCCCAAACTG TGTTCTGGGG ACAAGAGTGG CACTGTGGGC | |
| | | CGCAGGTCCC CTGAGCTCTC CTACCAGAAC TCCTCCGATC | |
| | | ACGCCAATGA GGAGTGGGAG ACGGCCTCCG AAAGCAGCGA | |
| | | CTTCAGCGAG CGGCGGGAGC GGCGGGAAGG CCCTGGGTCC | |
| | | GAGCCCGACT CCCAGGTGGA TGGTGGCCTG TCGGGGGCTA | |
| | | GTTTGGGTGA GAAGAAGGAG CTGGCCAAGA GGAGCTTCTC | |
| | | CAGTCAGAGA CCCGTGGTTG ACAGACAGAG CCGAAAGCTG | |
| | | GAGCCGGGAG GGTTTGGGGA GAAGCCCGTT AGGCCAGGTG | |
| | | GTGGTGACAC CTCCCCTCGC TATGAGAGCC AACAGAATGG | |
| | | GACGCCTTTG AAAGTGAAAA GATCCCCAGA CGAGGCCTTG | |
| | | CCTGGAGGTC TTAGTGGCTG CAGCAGTGGG AGTGGCCACT | |
| | | CCCCCTATGC CCTGGAGCGG GCAGCCCATG CCAGTGCTGA | |
| | | CCTTCCCGAA GCCTCCAGTA AAAAGGCAGA GAAGGAGGCC | |
| | | AAGTTGGCTG CTCCGAGGGC AGGTGAACAG GGAGAGGCCA | |
| | | TGAAACAGTT TGACCTGAAC TATGGAAGTG CCATCATTGA | |
| | | AAATTGCGGG TCCAGCCCCG GGGAGGAGAG TGAGGTGGGT | |
| | | TCTATGGTGG GCGAAGGCTT CATCGAAGTC CTGACCAAGA | |
| | | AGCAGCGCCG CCTGCTGGAG GAAGAGAGAA GAAAGAAGGA | |
| | | GCAGGCCGTG CAGGTGCCTG TCAAAGGTCG AGGCCTTTCC | |
| | | TCCCGTATTC CTCCTCGATT TGCAAAAAAG CAGAACAACT | |
| | | TATGTCTGGA GCAAGGTGAC GTGACCGTGC CTGGCAGCAG | |
| | | CCTGGGCACT GAGATCTGGG AGAGCAGCAG CCAGGCTCTC | |
| | | CCTGTGCAGG CCCCAGCCAA CGACTCCTGG AGGAAAGCTG | |
| | | TCACTGCCTT CAGCAGCACC GAGACTGGCT CTGCGGAGCA | |
| | | GGGTTTTAAG AGCAGCCAGG GAGATAGTGG CGTTGACTTG | |
| | | AGTGCCGAGT CTCGGGAGTC GTCTGCGACC TCCTCGCAGC | |
| | | GCAGCTCCCC ATATGGGACT CTGAAGCCAG AGGAGATGAG | |
| | | CGGGCCCGGC CTGGCGGAAC CCAAGGCCGA CAGCCACAAG | |
| | | GAGCAGGCTC CAAAGCCATC TGAGCAGAAG GATTCAGAAC | |
| | | AAGGCTCTGG ACAGAGCAAG GAGCACAGAC CAGGACCCAT | |
| | | CGGCAACGAG CGTTCTCTGA AAAACAGAAA GGGCTCGGAG | |
| | | GGGGCCGAGC GGCTGCAAGG GGCTGTCGTC CCGCCTGTTA | |
| | | ACGGGGTGGA GATTCACGTG GACTCCGTGC TGCCTGTGCC | |
| | | ACCCATTGAA TTTGGAGTCA GTCCAAAAGA CTCCGATTTC | |
| | | AGCTTGCCAC CTGGTTCTGC CTCTGGTCCT ACTGGGAGTC | |
| | | CAGTTGTTAA ACTTCAGGAT GCCTTGGCCA GTAATGCAGG | |
| | | GTTAACACAG AGTATCCCCA TCCTGCGGCG GGACCATCAC | |
| | | ATCCAGAGGG CCATCGGTCT CTCCCCAATG TCCTTCCCCA | |
| | | CCGCCGACCT TACTCTGAAG ATGGAGTCTG CGCGCAAGGC | |
| | | TTGGGAAAAC TCCCCCAGTT TGCCGGAGCA GAGCTCTCCA | |
| | | GGCGGCGCTG GCTCAGGCAT CCAGCCTCCA TCCTCTGTGG | |
| | | GTGCCTCCAG CGGGGTCAAC TACAGCTCCT TCGGTGGAGT | |
| | | GTCCATGCCA CCCATGCCTG TGGCCTCTGT AGCACCTTCT | |
| | | GCTTCTATGC CAGGCAGCCA CCTCCCGCCC CTGTACCTGG | |
| | | ATGGCCATGT GTTTGCAAGT CAGCCCCGGC TGGTTCCTCA | |
| | | AACGATACCT CAGCAGCAGA GTTACCAACA GGCCGCCGCT | |
| | | GCCCAGCAGA TCCCGATCTC CCTTCACACA TCTCTGCAGG | |
| | | CACAAGCTCA GCTTGGACTG AGGGGTGGGC TTCCTGTGTC | |
| | | CCAGTCCCAG GAGATCTTCA GCTCCTTGCA GCCCTTCAGA | |
| | | TCTCAGGTGT ACATGCACCC CAGCCTGTCA CCGCCCAGCA | |
| | | CCATGATCCT CTCTGGGGGC ACAGCCTTGA AGCCTCCATA | |
| | | CTCGGCGTTC CCAGGCATGC AGCCCTTGGA GATGGTGAAG | |
| | | CCGCAGTCTG GCTCACCCTA CCAGCCCATG AGCGGGAACC | |
| | | AAGCCCTGGT CTACGAGGGC CAGCTCAGCC AGGCTGCTGG | |
| | | CCTGGGTGCC TCCCAGATGT TGGACTCCCA GCTCCCACAG | |
| | | CTGACCATGC CACTGCCTCG GTACGGCTCC GGGCAGCAGC | |
| | | CACTGATCCT GCCCCAGTCT ATTCAGCTGC CACCTGGGCA | |
| | | GAGCCTCTCC GTTGGGGCCC CCCGAAGGAT TCCTCCGCCC | |
| | | GGGTCCCAGC CGCCAGTCCT GAACACCAGC AGAGAGCCCT | |
| | | CTCAGATGGA GATGAAAGGC TTCCACTTTG CCGACAGTAA | |
| | | ACAGAATGTC CCTTCAGGAG GCCCCGTGCC ATCGCCACAG | |
| | | ACCTACAGGC CTAGCTCTGC TAGCCCCAGT GGGAAGCCCT | |
| | | CTGGATCAGC AGTTAACATG GGCTCTGTGC AGGGACACTA | |
| | | CGTGCAACAG GCAAAACAAC GAGTGGATGA GAAACCCAGC | |
| | | CTGGGAGCCG TGAAGCTGCA GGAGGCCCCC TCGGCTGCCT | |
| | | CCCAGATGAA GCGAACCGGA GCGATCAAGC CTCGGGCTGT | |
| | | CAAAGTGGAG GAGAGTAAGG CCTGACAGTG CCTGGCTGCC | |
| | | ACCTCGCCTC TCCCTACTGA GGACGGTGCC GCCATGCGGC | |
| | | CTCGACACAG CCGACACTCG GGAGCCTCAC CAGATCCACC | |
| | | GTCCAAATGC GTGGCCCAGA CTGAGAGACC TCCCTCCTCT | |
| | | CCACTCCCGA AAGCTCCGTT GTCAACCAGC TTGCACCCGT | |
| | | GGATATATGG CATTGACCCG CTTGCTTTGA TACGAAACAA | |
| | | AAAAGCAGAC GACTCCTTCA TCCCATCTGC TCCTACCGTG | |
| | | ACTGTGGAGT GACGCCTCCT GTGCAGTGCA GATTTGCCCT | |
| | | CCCTGCCTCC TCCCTGTCCT GCCGCGCAGC CAGGGCGCCT | |
| | | TCTCAGCAGT GCTTCCGGCC CAGCCGCCCA TCCCTAGGCA | |
| | | CAGTGATTTG GCAGCAGGGT CATTTTACTT TGAGGCTTTT | |
| | | TGTTTTAAAA TGTAGCCAAG GTTTTTACAA AGGGGAAAGG | |
| | | AAAAGAAAAC AAAAACGCAA GCTCCATGTG TATAGCTGAA | |
| | | CTTTTATATG TTTCTTGCCA GCCCCTCCGC TCCCTTCCAT | |
| | | CTCTAGCCTC TGTCCTGTTT AGTTTGATAC GTCACTGCAG | |
| | | TACCTTAAGA GGTGACTCTT AAGAATGCAT CCCCTCCTGA | |
| | | TTCCTCAGCT GGTTCACCCT TGAGGTTATT TGCAAAAAGA | |
| | | AAAGGAGGTT CTTGAGGGCA CCGATTGCGA GCATTCTGGT | |
| | | GCCTGGCTCC CCGCCTGGGA AGCGATGGGG TGCTCAGAGC | |
| | | AGCAGGCAGG TTGGGGGAGG GGGGGGGTCA TAGTTGGGTT | |
| | | CCAGCTCCTG GCTTGATGAG CCCAGGGCGC TTACAGGCAG | |
| | | CCCATGAAGT TGATGACAGT TTTAGCATGA GAATCACACA | |
| | | GGGTCCCTGT CCTGGGCTCC TCTAAAGCCA GTGGATGTGC | |
| | | TGGGCACCAG AGACAAATCA TGGAGATGGC TGCTGGTGGC | |
| | | TCCCAGGTTG GCCCAGATGG GGTGAGCTGA CATACCACAG | |
| | | GCCCATCCCA GGCCCCGTGG GCTCTGCTTC TGGGGCTCCA | |
| | | TACCCTGCCC TGCAGGGGTG CTGTGTTTTT CACACATTTC | |
| | | TTTCCCTGAA GCCTTCTGTA ACCTGTCATT TTCCTTCCTT | |
| | | CCTCTTCCGG AGCCTGCTGC TTTCTCTGGA CCTGTCTCCA | |
| | | CCTCCCACAC AGCTCATCGT GAACACCACT TGGTGATGGA | |
| | | GGGAGTGGAC CCGTGTGTGG TCCCCAAGTG AGGCCACTGG | |
| | | GAGTTTGTCC TTTTCCTCCT TTGCTTCACT CCCAGCAGCA | |
| | | GACCCAGGTT GTCAGGACAG GAGGGCCTGA GCTAAGCAGT | |
| | | AGGCATCAGT CTCGTTTGTC TTCAGACGGC GGGGGCAGGT | |
| | | CCAGGGTGAG GCTGGGTGGA GGGCTGACCA AGGTCCAAAG | |
| | | GGCCTGCGCA GCCTCCGGGA GGGCAGCTTC TCCAGCCAGA | |
| | | GGCTTGTGTG AGCCATCGTG TGCTGGGCTT GTTTTTAAGT | |
| | | AAGAAACAAG GAAATCACTC CAGATTCTGT CATTCCAAGG | |
| | | AAAGGGAAGG GGACAGTTCA GGTTTCTCAG CTGTTCTTAG | |
| | | GGGTCACTGA GCGTCTACCT CCTCCTCCAG AGGAGGCTGG | |
| | | CTCAGAACAC CTAGAGGAGG GGGCCGGGGA TGCACCCCCC | |
| | | ACCAGAGGCT GCCTTCAGCG TCTCACGGGT GCAGGACAGC | |
| | | GCTCAGGCTT GGGCTCTAAG CTCTGTGTCT AGTGTAGAAC | |
| | | ATGGGGAAGG AGCATCTTAG GAACTGCTGA AGTAACTTCT | |
| | | TACTGCTCTC ACAATTCTAA GGAAGCGGGA GAACGGCCTC | |
| | | CTACCAACAG CGCCCACCCC AGAGCTGCCT GGGAAAGGGC | |
| | | AGTTTTACTG AAAGGTGCTT TACTGTTCAC CTGCATCTTT | |
| | | CAGCAGCTCC CCTCCTGCCC TCACCTGGTC TTTTCCCTCT | |
| | | TTATCCCAAG CCTTTATGCT TGAGTCCCTT CCCCAGGGGC | |
| | | TGCCCACCCG ACAGTTCCAG GCATTCCCTA CCTGAGCTTC | |
| | | TTGTCTGCTT TTCCTTCTCC CACTGCAAGC GGCTGCTTGT | |
| | | GGGGCCTGGG ATGAGCCCTC TCTGTCCCCA CCGGCCCTCC | |
| | | TTGCCAAGCC ATTCCTGGGT GAGTTCAGGC CTGCGGGAGC | |
| | | CACACATTCA TCTCCACCTG GACACTTGAG CCGCATGGCC | |
| | | AGACCCCTCC CACCTGATGC GGTGGTGCGT GTGATTTGTC | |
| | | AAAAGAAAGC CTTCTGGATG CTGTTAAGAT GTACCCTTCA | |
| | | GGTGAACCTG GTATCAGACC CACAGTACTT GCTGTTTGAG | |
| | | AAAAAATAAA AACAAAAAGG TCACCTGTTC TCCAGCCCTT | |
| | | TTCTCTTACC TGGTATTTCC TTCCTTTCTC CTCCCCCACC | |
| | | CCAAATAAAA AAACAAAAAA CACTAGAATT TATTTATATG | |
| | | TATTGATGTT GTAGGTCTAG GTGAAAAAAA AAGAAGTAAA | |
| | | TGTTTCACTG CTCTATTTAT ATATAATGTC TGAATTAATT | |
| | | CTGTGCAGGA AAGGCCAGGA AATTGCATGT GAAGTTCGGT | |
| | | GCAGTCACCA CCTGTGTGTG ACCTGAGCTG CAGTCTCTTC | |
| | | GCTGAGATGC AGGTTTTAAA TGAGACTTGG GGGGCTGAGG | |
| | | GCAGGCCTCA GGCCTCCCAG CGCCCCAACC CCTCCTTGGT | |
| | | CTAATGAAAT GCAGTTCTTA GTGCAGAGAT GTTTTAAGGT | |
| | | GCAATATATC TCTTCCTTTC CCGTGGTTTT AGAGCCAAGC | |
| | | TCAAGGTAGT AGGACGTAGG GTCTTATTTT GTTTTCAAAC | |
| | | CCCCATCCTC AGAGCGCAGA TACATGCAGA GGCTTCTGCC | |
| | | AGGATACCAC GGGGCCTTAG TGGGAACAGG TGGAGACCAG | |
| | | CACTTCCCTT TCCTGCTGCT GAGGTAGGGA TTGGGGGGTC | |
| | | AGAACCCACT CACTTTTGCC TGTTAAAGTT GCCCTCCTGA | |
| | | CGCTGGCAGC TCTGCCTTGG TCACTGGGGA TGCGGCTCGT | |
| | | TGCTCAGCCA CCAGTGGCCT TGCGGTATTG TCCACCATCC | |
| | | ACTAGAGTGG GATGAAGTCC AGAGTGTGGG TATACATCTC | |
| | | AGATGCCCAT CTACCCACTG GGGACTTCAA TGCCAGCTGC | |
| | | ATTTGGTTTG GTTTTCTTAA CTGTTGGCTT CTCCCCACAG | |
| | | CGTTTTTTGT TTTTTTTTAA ACATTCATAT TGTTTTCAAA | |
| | | CTTGGAATTC ATAGACACTC TGGCTCTAGG TTCCTTAAGG | |
| | | GGGAAAACAA AAGATGACTT TATTTCACAT TCAAGAAAAT | |
| | | CAGTTCAGTT CCAAAGCTGT GGTCCTTCCA GCCACTTCTA | |
| | | GGGACACTGG GGAACCTTGT TAAACGTTGA CATCAGTGCT | |
| | | CTCCAGCCGT GCTGTCACCC TCCTATCTTC TGGATCTGCC | |
| | | TTCGCGATGG TCAGTGACAG CTTCTGGAAG CTGAGCACAC | |
| | | ACAGGTGCAC AGCCATGCTG TGGTCTGGCC TGCTACGGCA | |
| | | GCATGGCAGC TCTGGTGGAG CCTTCTCCCT TGCCATTTGG | |
| | | TTCCCCTGTG CCAAGTAGCT GCAGGCTGCC CCTCAAATCT | |
| | | TCATTTGTCC CTTTTCACTT CCTGCAGAAC AAGCCTGGGT | |
| | | TAGAGGGTCT GCTGGAAATG GCCTTTGAAG ACCAAGGATA | |
| | | CCAGGATGTG TGCACTCTGT CGTGTTCTGT GATGAATGGG | |
| | | AAACGTAGGC TTCCAGAAAG CCAGCTCTCT TCTGAAATGT | |
| | | GACGGACCTA AGCAGGAAGT CATCCAGGAC AGGAGTGGCT | |
| | | CAGTGTTGGG GATGGACGCT GTCGCCCAGC CATGCTCCAC | |
| | | CAGGGCCACC AATGTGTAGT TGGCTGGTGG TCTTCGGGCA | |
| | | TGTGAGACCT GCTCTTCACT GTTTCCACCC CACTTGGTGG | |
| | | CCTCCAGGAT GGTAGTGGCA CCCTCAGAGC CCCATCTTCA | |
| | | GCATGTTCTG AAGCCTCAGA GTGGAAATTC CTGCTAAGGC | |
| | | TCTGTGTGGA CGCCTTTCTC CCGTGATCTA AAGGGGACAC | |
| | | TGTACTCAAG CTTTTGACCT CATGCCTTGT GTAGTAAAAA | |
| | | AGGATTTGGG GGTTTTGTTT GGTTCCTGAG AGGGTTGTGT | |
| | | TTTGTTTTTG TTTCCTTTTG TTTATGTTTT GGCCTTTCCT | |
| | | CTTTGTCTTT CCATGTAGAC CAGATATTTG AAAGGGCAGA | |
| | | CGATGGCTAG AGGTGTAATG TGCAGCTTGT TTATACGGTA | |
| | | TTTTGGGAAA CTTACCTTGG ATGGGAAATC GAATCGTGGA | |
| | | TTCACCAGGC CGGTGCTGGC ACACTCACCC TCGCCCTTTC | |
| | | CCTCCGGTTC AGTACCTATT GTTTCTCCTT TCAAATATGT | |
| | | GATTGTACTA GCTCTTTCCA TATGAAAGAA TTCTCCTTAT | |
| | | TTAAATAAAA AAAGTTTAAA AA | |
| DOPEY2 | NM_005128.3 | TCCCACAGTG CCTGGCCCAG AAGCCTTGCT AAATATTTGA | 33 |
| | | ACAGGATTGC CCAATACTTT TCTGCTGTGA GAATGTAAGA | |
| | | TGGATCCAGA AGAGCAGGAG CTCTTAAATG ATTACAGATA | |
| | | CAGAAGCTAC TCTTCAGTGA TTGAAAAGGC TTTGAGAAAT | |
| | | TTTGAGTCCT CGAGTGAATG GGCGGATCTC ATATCTTCAC | |
| | | TTGGCAAACT CAACAAGGCT CTTCAGAGTA ACCTGAGGTA | |
| | | CTCCTTGTTG CCAAGACGGC TCCTCATCAG CAAAAGATTA | |
| | | GCTCAGTGTT TGCACCCTGC CCTGCCCAGT GGTGTCCACT | |
| | | TAAAAGCTCT GGAAACCTAC GAGATTATCT TTAAAATCGT | |
| | | GGGGACCAAA TGGCTGGCCA AGGACTTGTT TCTGTACAGC | |
| | | TGCGGGTTAT TTCCTCTCCT GGCACACGCG GCGGTGTCGG | |
| | | TGAGGCCGGT GCTGCTCACC CTGTACGAGA AGTACTTCCT | |
| | | CCCACTGCAG AAGCTGCTCC TGCCCAGTCT GCAGGCCTTC | |
| | | ATCGTGGGCC TGCTGCCCGG CCTTGAAGAG GGCTCCGAGA | |
| | | TCTCCGACAG AACGGATGCT CTGCTCCTGA GACTGTCGCT | |
| | | GGTGGTTGGC AAAGAGGTGT TTTACACCGC CCTCTGGGGG | |
| | | AGCGTCCTGG CCAGCCCGTC CATCCGCCTC CCTGCCTCAG | |
| | | TCTTCGTGGT GGGCCACATC AACAGGGATG CCCCCGGCCG | |
| | | GGAGCAGAAG TACATGCTGG GGACCAATCA CCAACTCACG | |
| | | GTGAAGTCTT TGCGTGCCTC CCTGTTGGAC TCAAATGTTC | |
| | | TTGTGCAAAG AAATAATCTG GAAATCGTTC TGTTTTTCTT | |
| | | CCCATTTTAT ACCTGTCTGG ATTCCAATGA GAGAGCCATC | |
| | | CCCCTCCTCA GATCTGACAT CGTGCGCATT CTCTCAGCCG | |
| | | CCACCCAGAC CCTACTGAGA AGGGACATGT CCCTGAACAG | |
| | | AAGACTGTAT GCATGGTTAC TAGGCTCAGA CATAAAAGGA | |
| | | AATACCGTTG TGCCAGAATC TGAAATCTCA AATTCTTATG | |
| | | AAGACCAGTC GTCTTATTTT TTTGAAAAAT ACTCCAAGGA | |
| | | TCTTTTAGTT GAGGGTTTGG CTGAGATATT GCATCAGAAG | |
| | | TTCATAGATG CTGACGTGGA GGAACGCCAT CATGCATACC | |
| | | TGAAGCCTTT TCGCGTCCTC ATCAGTCTGC TTGACAAGCC | |
| | | AGAAATAGGG CCTCAAGTGG TTGGGAATTT GTTTCTCGAA | |
| | | GTCATCAGGG CCTTTTATTC TTACTGCAGA GATGCCCTTG | |
| | | GCTCTGATCT TAAACTTAGC TACACCCAGA GTGGAAATTC | |
| | | GCTGATAAGT GCAATCAAGG AAAACAGAAA TGCCTCTGAG | |
| | | ATTGTCAAAA CGGTAAATTT GCTGATAACT TCTCTAAGCA | |
| | | CAGACTTTCT CTGGGATTAT ATGACAAGGT GTTTTGAGGA | |
| | | ATGCTTTAGA CCAGTGAAGC AGCGTTACAG CGTGAGGAAC | |
| | | AGCGTCAGCC CTCCCCCCAC GGTCTCGGAG CTCTGCGCCC | |
| | | TCCTGGTCTT CCTGCTGGAT GTCATTCCTT TGGAACTTTA | |
| | | CTCTGAGGTG CAAACCCAGT ATCTCCCTCA GGTGCTCGGC | |
| | | TGCCTGGTGC AGCCTCTTGC TGAGGACATG GAGGCCTTAA | |
| | | GTTTACCTGA ACTCACGCAT GCCTTGAAGA CGTGTTTCAA | |
| | | GGTGCTCAGC AAAGTCCAGA TGCCTCCTTC CTACCTCGAC | |
| | | ACGGAGTCCA CCAGCGGAAC CTCGAGTCCA GTAAAAGGTG | |
| | | AAAACGGCAA AATAATTTTG GAAACAAAGG CAGTGATTCC | |
| | | CGGTGACGAA GATGCTTCGT TTCCCCCTCT GAAGTCTGAG | |
| | | GACAGTGGGA TCGGGCTCAG TGCCTCGTCA CCGGAGCTCT | |
| | | CTGAGCACTT GAGGGTTCCT CGAGTTTCTC TGGAAAGGGA | |
| | | CGACGTTTGG AAGAAGGGCG GGAGCATGCA GAGGACGTTT | |
| | | CTTTGCATCC AAGAGCTAAT CGCCAACTTT GCCAGCAAGA | |
| | | ACATTTTTGG AGTACAGCTG ACAGCGTCAG GAGAAGAAAG | |
| | | CAAGTCCGAG GAGCCTGCAG GGAAGAGGGA CAGGGATGGG | |
| | | ACGCAGAGCC TGGCAGCCAA TGATTCCAGC AGGAAGAACT | |
| | | CTTGGGAGCC CAAGCCCATC ACTGTGCCTC AGTTCAAGCA | |
| | | GATGCTGTCA GACTTGTTCA CAGCACGAGG GTCTCCATTC | |
| | | AAGACAAAAA GTTCAGAGTC ACCATCGTCT TCGCCCAGCA | |
| | | GCCCTGCCAG GAAAAACGGG GGAGAATGGG ATGTTGAGAA | |
| | | GGTGGTCATT GACCTGGGGG GTTCCAGGGA GGAACGCAGG | |
| | | GAGGCCTTTG CCGCCGCCTG CCACCTGCTG CTGGATTGTG | |
| | | CCACTTTCCC TGTCTACCTG TCCGAGGAAG AGACCGAGCA | |
| | | GCTCTGTGCA ACGCTCTTCC AGCTGCCAGG AGCCGGTGAT | |
| | | TCCAGTTTTC CATCTTGGCT GAAGTCCCTC ATGACTATTT | |
| | | GCTGCTGTGT GACTGACTGC TACCTCCAGA ACGTGGCCAT | |
| | | TTCCACTCTG CTGGAAGTGA TAAACCATTC CCAGTCCCTG | |
| | | GCGCTTGTCA TTGAAGACAA GATGAAACGC TATAAGAGCT | |
| | | CTGGACACAA CCCTTTTTTT GGCAAGCTGC AGATGGTGAC | |
| | | GGTTCCTCCC ATTGCTCCAG GGATATTGAA AGTCATTGCA | |
| | | GAGAAAACAG ATTTCTATCA GAGGGTGGCT CGTGTGCTTT | |
| | | GGAATCAGCT GAACAAAGAG ACCCGGGAGC ATCACGTCAC | |
| | | CTGCGTAGAA TTGTTCTACC GGCTGCACTG CCTGGCCCCT | |
| | | ACGGCCAACA TCTGCGAGGA CATCATCTGC CATGCCCTCC | |
| | | TGGACCCTGA CAAGGGAACA AGGCTGGAAG CTCTGTTTAG | |
| | | ATTTTCCGTG ATCTGGCATC TGACAAGAGA GATCCAAGGC | |
| | | AGTCGAGTAA CATCTCACAA TCGCTCCTTT GATAGGTCCT | |
| | | TGTTTGTCGT GCTGGACAGC CTGGCCTGCA CGGATGGTGC | |
| | | CATCGGTGCG GCAGCCCAGG GCTGGCTGGT GCGTGCGCTC | |
| | | TCCCTCGGGG ACGTGGCTCG CATCCTCGAA CCCGTGCTCC | |
| | | TGCTGCTGCT GCAGCCAAAA ACCCAGAGAA CCTCCATCCA | |
| | | CTGCCTCAAG CAGGAGAACT CGGCCGATGA CTTGCACCGT | |
| | | TGGTTTAACA GGAAGAAAAC CTCTTTCAGA GAGGCATGCG | |
| | | CAGTGCCCGA GCCTCAGGAG AGCGGCTCTG AAGAGCACCT | |
| | | GCCTCTGAGC CAGTTCACCA CAGTGGACCG TGAAGCCATT | |
| | | TGGGCCGAAG TGGAGAAGGA GCCCGAGAAG TACCCGCTGC | |
| | | GAGGCGAGCT GAGCGAGGAA GAGCTGCCCT ACTACGTGGA | |
| | | GCTTCCAGAC AGGACGGCCC ACGGCGCCCC GGACAGCAGC | |
| | | GAGCACACCG AGTCTGCAGA TACAAGCTCC TGCCACACGG | |
| | | ACAGCGAGAA CACGTCCTCC TTCTCCTCCC CTTCCCACGA | |
| | | CCTGCAGGAG CTGAGCAACG AAGAGAACTG CTGTGCACCC | |
| | | ATCCCCATGG GGGGCAGGGC GTACCCCAAG CGCTCGGCCC | |
| | | TGCTGGCGGC CTTCCAGTCA GAAAGCTTCA AGGCTGGGGC | |
| | | CAAGTTAAGC CTGGTGCGGG TGGACTCGGA CAAGACGCAG | |
| | | GCTTCTGAGT CGTTCTCCAG CGACGAGGAG GCGGACTTGG | |
| | | AGCTCCAGGC CCTCACCACA TCCAGGCTGC TAAAGCAGCA | |
| | | GCGGGAAAGG CAGGAGGCCG TCGAGGCCTT GTTCAAGCAC | |
| | | ATCCTGCTCT ACCTGCAGCC CTACGACTCT CGGCGGGTCC | |
| | | TCTATGCCTT CTCGGTGCTG GAGGCTGTGC TCAAAACCAA | |
| | | CCCTAAGGAA TTCATCGAGG CTGTGTCCAG GACTAGCATG | |
| | | GATACCAGCT CCACCGCGCA CCTCAACCTC ATCTCCAACC | |
| | | TCCTCGCTCG CCACCAGGAG GCCCTCATTG GCCAGAGTTT | |
| | | CTACGGAAAG CTCCAGACCC AGGTCCCCAA CGTGTGCCCC | |
| | | CACTCTCTGC TCCTGGAGCT GCTCACCTAC CTCTGCCTGA | |
| | | GCTTCCTGCG CTCCTACTAC CCTTGCTATT TGAAGGTCTC | |
| | | GCACCGAGAC ATTCTCGGCA ACCGGGACGT GCAGGTCAAA | |
| | | AGTGTCGAGG TTTTGATCAG GATAATGATG CAGCTGGTCT | |
| | | CAGTGGCCAA GTCTTCGGAA GGGAAGAACG TGGAGTTCAT | |
| | | CCACAGCTTG CTGCAGAGGT GCAAAGTTCA GGAGTTTGTC | |
| | | CTGCTCTCCC TGTCGGCGTC CATGTACACG AGCCAGAAGC | |
| | | GCTACGGGCT GGCCACCGCC CACCACGGCA GGGCCCTGCC | |
| | | AGAGGACAGC CTCTTTGAGG AGAGTCTCAT TAACTTGGGT | |
| | | CAGGACCAGA TCTGGAGTGA GCACCCGCTG CAGATTGAGC | |
| | | TGCTGAAGCT GCTGCAGGTG CTGATTGTCT TGGAACACCA | |
| | | CCTGGGTCGG GCCCATGAGG AGGCGGAAAA CCAGCCCGAC | |
| | | CTGTCCCGGG AGTGGCAGAG AGCCCTGAAC TTCCAGCAGG | |
| | | CCATCAGCGC CCTGCAGTAC GTGCAGCCCC ACCCCCTCAC | |
| | | CTCCCAGGGT CTTCTGGTCT CTGCGGTGGT GAGGGGTCTG | |
| | | CAGCCCGCCT ACGGTTACGG CATGCATCCG GCCTGGGTGA | |
| | | GCTTGGTCAC GCATTCCTTG CCCTACTTCG GAAAGTCCCT | |
| | | GGGCTGGACG GTGACACCCT TTGTTGTCCA GATTTGCAAA | |
| | | AACTTGGATG ACTTGGTCAA GCAGTATGAA AGCGAATCTG | |
| | | TGAAGCTCTC TGTCAGCACA ACCTCCAAGA GGGAAAACAT | |
| | | TTCTCCAGAT TATCCACTCA CCCTTCTAGA AGGTCTAACG | |
| | | ACCATTAGTC ATTTTTGTCT TTTGGAACAA GCCAACCAAA | |
| | | ACAAAAAGAC CATGGCTGCA GGTGATCCTG CCAACTTGAG | |
| | | GAATGCCAGA AATGCCATTT TGGAAGAGCT GCCTCGAACT | |
| | | GTTAACACCA TGGCCCTTCT CTGGAATGTT CTCAGAAAGG | |
| | | AGGAGACTCA AAAGAGACCT GTCGATCTCC TAGGGGCCAC | |
| | | GAAGGGATCC TCTTCCGTTT ACTTTAAAAC CACCAAAACC | |
| | | ATAAGACAAA AAATTTTAGA CTTCTTAAAC CCCTTGACGG | |
| | | CCCATCTTGG GGTTCAGTTG ACAGCGGCTG TTGCGGCAGT | |
| | | GTGGAGCAGA AAGAAAGCCC AGCGTCACAG TAAGATGAAG | |
| | | ATTATCCCAA CGGCAAGTGC ATCCCAGCTA ACCCTTGTCG | |
| | | ACTTGGTGTG TGCACTCAGC ACCCTGCAGA CTGACACGCT | |
| | | GCTGCACCTG GTGAAGGAGG TGGTGAAGAG GCCACCCCAA | |
| | | GTCAAAGGGG GTGATGAGAA ATCGCCCCTA GTGGACATTC | |
| | | CTGTGTTGCA GTTTTGCTAT GCTTTTCTCC AAAGGCTCCC | |
| | | AGTACCAGCC TTGCAAGAGA ACTTTTCTTC ACTGTTGGGA | |
| | | GTATTGAAAG AGTCTGTACA GTTGAATCTA GCCCCACCTG | |
| | | GGTATTTTCT GCTTCTCAGC ATGCTGAATG ACTTTGTAAC | |
| | | AAGAACTCCC AACCTGGAAA ACAAGAAGGA CCAAAAAGAC | |
| | | CTGCAGGAAA TCACTCAGAA AATCCTAGAA GCTGTGGGGA | |
| | | ACATTGCCGG CTCTTCCTTG GAGCAAACCA GCTGGCTAAG | |
| | | CAGAAACCTG GAAGTGAAGG CCCAACCTCA GGCCTCTCTA | |
| | | GAAGAATCTG ATGCTGAGGA GGACCTGTAT GATGCTGCTG | |
| | | CAGCTTCAGC AATGGTGTCT TCATCCGCCC CGTCGGTGTA | |
| | | CAGCGTGCAA GCCCTCTCTC TCCTGGCAGA GGTACTGGCT | |
| | | TCCCTCCTGG ACATGGTTTA TCGAAGTGAT GAGAAGGAGA | |
| | | AAGCTGTGCC GTTAATCTCC CGTCTGCTTT ACTATGTTTT | |
| | | TCCATACTTA CGCAACCACA GTGCCTACAA TGCTCCCAGC | |
| | | TTCCGGGCTG GCGCTCAGCT GCTGAGCTCC CTGAGTGGCT | |
| | | ATGCCTACAC AAAGCGAGCC TGGAGGAAGG AGGTCCTGGA | |
| | | GCTGTTTCTC GACCCCGCTT TCTTTCAGAT GGATACTTCC | |
| | | TGTGTTCATT GGAAGTCCAT TATTGACCAT CTTTTGACTC | |
| | | ATGAGAAAAC AATGTTTAAG GATTTAATGA ACATGCAGAG | |
| | | CAGTTCTTTG AAACTATTCT CAAGTTTTGA ACAGAAAGCC | |
| | | ATGCTGTTAA AGCGCCAGGC TTTTGCTGTC TTCAGTGGAG | |
| | | AACTTGATCA ATACCACCTT TACCTTCCAC TGATACAAGA | |
| | | ACGCCTGACA GACAATCTCA GAGTTGGACA GACATCCATA | |
| | | GTTGCTGCTC AGATGTTTCT TTTTTTCAGA GTTTTGCTGC | |
| | | TAAGAATATC TCCTCAACAT TTGACTTCAT TGTGGCCAAT | |
| | | AATGGTCTCT GAATTGATTC AGACATTCAC ACAGCTTGAA | |
| | | GAAGATCTAA AAGATGAAGA TGAGTCATTG AGAAGCACCA | |
| | | ACAAAGTAAA CAGAACGAAA GTTTCAGTCC CGGATGCAAA | |
| | | TGGACCCTCA GTGGGGGAGA TACCCCAGAG TGAACTCATC | |
| | | TTGTATTTAT CAGCTTGCAA ATTCTTGGAC ACAGCGCTTT | |
| | | CTTTTCCACC TGACAAGATG CCATTATTTC AAATTTATAG | |
| | | GTGGGCATTT ATTCCAGAAG TGGACACAGA GGGCCCTGCC | |
| | | TTCCTGTCGG ATGTAGAGGA GAATCACCAA GAATGCAAAC | |
| | | CCCACACTGT CAGGATTCTA GAACTTCTAA AATTAAAGTT | |
| | | TGGGGAAATC AGTAGCTCTG ATGAGATCAC CATGAAGAGT | |
| | | GAATTCCCGC TTCTGCGCCA ACATTCTGTT TCCAGCATCA | |
| | | GGCAGTTGAT GCCATTCTTC ATGACTCTAA ATGGTGCATT | |
| | | TAAGACCCAG AGACAGCTGC CTGCTGATAG CCCAGGAACT | |
| | | CCATTCTTGG ACTTTCCTGT CACAGATAGC CCAAGGATCT | |
| | | TAAAACAACT GGAAGAATGC ATCGAATATG ATTTTCTGGA | |
| | | ACATCCAGAA TGTTAACCAT GTGAGAGAGA ATATGTTTAA | |
| | | TCCATGTATT GGTACTTTAC TGAAAACCAG GTTATATTCT | |
| | | AAAGAAGAAA GAAGGCAGGA TAGTGCTTTT GAACAAGCCT | |
| | | ATTTCCATTT TGAAAGTAGA TTTCAGGCTA GGTGCGGTGG | |
| | | CTCACACCTG TAATCTCAGC ACTTTGGGAG GCCAAGGCAG | |
| | | GCAGATCACT TGAGGTCAGG AGTTCGAGAC CAGCCTGACC | |
| | | AACATGGTGA GACCCTGTCT CTACTAAAAA TACAAAAATT | |
| | | AGCTGGGTGT GGTGGCGGCG CCTGTAATCC CAGCTACTTG | |
| | | GGAGGCTAAG GCATGAGAAT TGCTTGAACC CAGGAGGTGG | |
| | | AGGCTGCAGT GAGCCGAGAT CACGACACTG CACTCCAGCT | |
| | | GTGTGACAGA ATGAGACCAT CTCCAAAAAA AAAAAAAAGT | |
| | | AGATTTCAGA TAATTTACTG TTCAGCAACA GGACACACCT | |
| | | CCCTAAATGC CTTGTAATAT ATTTGAATCT GATTCTGCAT | |
| | | TTCTTCCTCA ATTTATGTAA TGAAAATAAA ATTAATATAT | |
| | | CATCTAACAG TAGCACAAAA TTTGTAATAT GAAGTAAAGT | |
| | | ATGAAGATAA TGAAGAAGTT GTTTTCTTTG TTGAAGCAGT | |
| | | TATATGGGTC TTTCTCAGTA TATTTCTCTT TTCTCTAAAA | |
| | | GTTTAAACTT ATTAAAAGAA TGTTATTTTT AACCTTTCAA | |
| | | AAAAAAAAA | |
| NDUFB11 | NM_019056.6 | GCTCTGGCCG GCCCCGGCGA TTGGTCACCG CCCGCTAGGG | 34 |
| | | GACAGCCCTG GCCTCCTCTG ATTGGCAAGC GCTGGCCACC | |
| | | TCCCCACACC CCTTGCGAAC GCTCCCCTAG TGGAGAAAAG | |
| | | GAGTAGCTAT TAGCCAATTC GGCAGGGCCC GCTTTTTAGA | |
| | | AGCTTGATTT CCTTTGAAGA TGAAAGACTA GCGGAAGCTC | |
| | | TGCCTCTTTC CCCAGTGGGC GAGGGAACTC GGGGCGATTG | |
| | | GCTGGGAACT GTATCCACCC AAATGTCACC GATTTCTTCC | |
| | | TATGCAGGAA ATGAGCAGAC CCATCAATAA GAAATTTCTC | |
| | | AGCCTGGCCG AAAATGGTTG GCCCCACGAA GCCACGACAA | |
| | | CTGGAGGCAA AGAGGGTTGC TCAACGCCCC GCCTCATTGG | |
| | | AAAACCAAAT CAGATCTGGG ACCTATATAG CGTGGCGGAG | |
| | | GCGGGGCGAT GATTGTCGCG CTCGCACCCA CTGCAGCTGC | |
| | | GCACAGTCGC ATTTCTTTCC CCGCCCCTGA GACCCTGCAG | |
| | | CACCATCTGT CATGGCGGCT GGGCTGTTTG GTTTGAGCGC | |
| | | TCGCCGTCTT TTGGCGGCAG CGGCGACGCG AGGGCTCCCG | |
| | | GCCGCCCGCG TCCGCTGGGA ATCTAGCTTC TCCAGGACTG | |
| | | TGGTCGCCCC GTCCGCTGTG GCGGGAAAGC GGCCCCCAGA | |
| | | ACCGACCACA CCGTGGCAAG AGGACCCAGA ACCCGAGGAC | |
| | | GAAAACTTGT ATGAGAAGAA CCCAGACTCC CATGGTTATG | |
| | | ACAAGGACCC CGTTTTGGAC GTCTGGAACA TGCGACTTGT | |
| | | CTTCTTCTTT GGCGTCTCCA TCATCCTGGT CCTTGGCAGC | |
| | | ACCTTTGTGG CCTATCTGCC TGACTACAGG TGCACAGGGT | |
| | | GTCCAAGAGC GTGGGATGGG ATGAAAGAGT GGTCCCGCCG | |
| | | CGAAGCTGAG AGGCTTGTGA AATACCGAGA GGCCAATGGC | |
| | | CTTCCCATCA TGGAATCCAA CTGCTTCGAC CCCAGCAAGA | |
| | | TCCAGCTGCC AGAGGATGAG TGACCAGTTG CTAAGTGGGG | |
| | | CTCAAGAAGC ACCGCCTTCC CCACCCCCTG CCTGCCATTC | |
| | | TGACCTCTTC TCAGAGCACC TAATTAAAGG GGCTGAAAGT | |
| | | CTGAAAAAAA AAAAAAAA | |
| ND4 | NC_012920.1 | ATGCTAAAACTAATCGTCCCAACAATTATATTACTACCACTGAC | 35 |
| | | ATGACTTTCCAAAAAACACATAATTTGAATCAACACAACCACCC | |
| | | ACAGCCTAATTATTAGCATCATCCCTCTACTATTTTTTAACCAAA | |
| | | TCAACAACAACCTATTTAGCTGTTCCCCAACCTTTTCCTCCGACC | |
| | | CCCTAACAACCCCCCTCCTAATACTAACTACCTGACTCCTACCC | |
| | | CTCACAATCATGGCAAGCCAACGCCACTTATCCAGTGAACCACT | |
| | | ATCACGAAAAAAACTCTACCTCTCTATACTAATCTCCCTACAAA | |
| | | TCTCCTTAATTATAACATTCACAGCCACAGAACTAATCATATTT | |
| | | TATATCTTCTTCGAAACCACACTTATCCCCACCTTGGCTATCATC | |
| | | ACCCGATGAGGCAACCAGCCAGAACGCCTGAACGCAGGCACAT | |
| | | ACTTCCTATTCTACACCCTAGTAGGCTCCCTTCCCCTACTCATCG | |
| | | CACTAATTTACACTCACAACACCCTAGGCTCACTAAACATTCTA | |
| | | CTACTCACTCTCACTGCCCAAGAACTATCAAACTCCTGAGCCAA | |
| | | CAACTTAATATGACTAGCTTACACAATAGCTTTTATAGTAAAGA | |
| | | TACCTCTTTACGGACTCCACTTATGACTCCCTAAAGCCCATGTC | |
| | | GAAGCCCCCATCGCTGGGTCAATAGTACTTGCCGCAGTACTCTT | |
| | | AAAACTAGGCGGCTATGGTATAATACGCCTCACACTCATTCTCA | |
| | | ACCCCCTGACAAAACACATAGCCTACCCCTTCCTTGTACTATCC | |
| | | CTATGAGGCATAATTATAACAAGCTCCATCTGCCTACGACAAAC | |
| | | AGACCTAAAATCGCTCATTGCATACTCTTCAATCAGCCACATAG | |
| | | CCCTCGTAGTAACAGCCATTCTCATCCAAACCCCCTGAAGCTTC | |
| | | ACCGGCGCAGTCATTCTCATAATCGCCCACGGGCTTACATCCTC | |
| | | ATTACTATTCTGCCTAGCAAACTCAAACTACGAACGCACTCACA | |
| | | GTCGCATCATAATCCTCTCTCAAGGACTTCAAACTTACTCCCAC | |
| | | TAATAGCTTTTTGATGACTTCTAGCAAGCCTCGCTAACCTCGCC | |
| | | TTACCCCCCACTATTAACCTACTGGGAGAACTCTCTGTGCTAGT | |
| | | AACCACGTTCTCCTGATCAAATATCACTCTCCTACTTACAGGAC | |
| | | TCAACATACTAGTCACAGCCCTATACTCCCTCTACATATTTACC | |
| | | ACAACACAATGGGGCTCACTCACCCACCACATTAACAACATAA | |
| | | AACCCTCATTCACACGAGAAAACACCCTCATGTTCATACACCTA | |
| | | TCCCCCATTCTCCTCCTATCCCTCAACCCCGACATCATTACCGGG | |
| | | TTTTCCTCTT | |
| MORF4L1 | NM_001265603.1 | CGGCGTGCCC TGGGGCGGCG CGGGCGCAGG GGCGCGTGCG | 36 |
| | | CGGCGGGCTG TCGTTGGCTG GAGCAGCGGC TGCGCGGGTC | |
| | | GCGGTGCTGT GAGGTCTGCG GGCGCTGGCA AATCCGGCCC | |
| | | AGGATGTAGA GCTGGCAGTG CCTGACGGCG CGTCTGACGC | |
| | | GGAGTTGGGT GGGGTAGAGA GTAGGGGGCG GTAGTCGGGG | |
| | | GTGGTGGGAG AAGGAGGAGG CGGCGAATCA CTTATAAATG | |
| | | GCGCCGAAGC AGGACCCGAA GCCTAAATTC CAGGAGGTTG | |
| | | GGATGAATGG GTTCCGGAGA GCAGAGTACT CAAATACGTG | |
| | | GACACCAATT TGCAGAAACA GCGAGAACTT CAAAAAGCCA | |
| | | ATCAGGAGCA GTATGCAGAG GGGAAGATGA GAGGGGCTGC | |
| | | CCCAGGAAAG AAGACATCTG GTCTGCAACA GAAAAATGTT | |
| | | GAAGTGAAAA CGAAAAAGAA CAAACAGAAA ACACCTGGAA | |
| | | ATGGAGATGG TGGCAGTACC AGTGAGACCC CTCAGCCTCC | |
| | | TCGGAAGAAA AGGGCCCGGG TAGATCCTAC TGTTGAAAAT | |
| | | GAGGAAACAT TCATGAACAG AGTTGAAGTT AAAGTAAAGA | |
| | | TTCCTGAAGA GCTAAAACCG TGGCTTGTTG ATGACTGGGA | |
| | | CTTAATTACC AGGCAAAAAC AGCTCTTTTA TCTTCCTGCC | |
| | | AAGAAGAATG TGGATTCCAT TCTTGAGGAT TATGCAAATT | |
| | | ACAAGAAATC TCGTGGAAAC ACAGATAATA AGGAGTATGC | |
| | | GGTTAATGAA GTTGTGGCAG GGATAAAAGA ATACTTCAAC | |
| | | GTAATGTTGG GTACCCAGCT ACTCTATAAA TTTGAGAGAC | |
| | | CACAGTATGC TGAAATTCTT GCAGATCATC CCGATGCACC | |
| | | CATGTCCCAG GTGTATGGAG CGCCACATCT CCTGAGATTA | |
| | | TTTGTACGAA TTGGAGCAAT GTTGGCTTAT ACACCTCTGG | |
| | | ATGAGAAGAG CCTTGCTTTA TTACTCAATT ATCTTCACGA | |
| | | TTTCCTAAAG TACCTGGCAA AGAATTCTGC AACTTTGTTC | |
| | | AGTGCCAGCG ATTATGAAGT GGCTCCTCCT GAGTACCATC | |
| | | GGAAAGCTGT GTGAGAGGCA CTCTCACTCA CTTATGTTTG | |
| | | GATCTCCGTA AACACATTTT TGTTCTTAGT CTATCTCTTG | |
| | | TACAAACGAT GTGCTTTGAA GATGTTAGTG TATAACAATT | |
| | | GATGTTTGTT TTCTGTTTGA TTTTAAACAG AGAAAAAATA | |
| | | AAAGGGGGTA ATAGCTCCTT TTTTCTTCTT TCTTTTTTTT | |
| | | TTTCATTTCA AAATTGCTGC CAGTGTTTTC AATGATGGAC | |
| | | AACAGAGGGA TATGCTGTAG AGTGTTTTAT TGCCTAGTTG | |
| | | ACAAAGCTGC TTTTGAATGC TGGTGGTTCT ATTCCTTTGA | |
| | | CACTACGCAC TTTTATAATA CATGTTAATG CTATATGACA | |
| | | AAATGCTCTG ATTCCTAGTG CCAAAGGTTC AATTCAGTGT | |
| | | ATATAACTGA ACACACTCAT CCATTTGTGC TTTTGTTTTT | |
| | | TTTTATGGTG CTTAAAGTAA AGAGCCCATC CTTTGCAAGT | |
| | | CATCCATGTT GTTACTTAGG CATTTTATCT TGGCTCAAAT | |
| | | TGTTGAAGAA TGGTGGCTTG TTTCATGGTT TTTGTATTTG | |
| | | TGTCTAATGC ACGTTTTAAC ATGATAGACG CAATGCATTG | |
| | | TGTAGCTAGT TTTCTGGAAA AGTCAATCTT TTAGGAATTG | |
| | | TTTTTCAGAT CTTCAATAAA TTTTTTCTTT AAATTTCAAA | |
| | | GAACAAAAAA AAAAAAAAA | |
| MRPL19 | NM_014763.3 | GTAGTCTTGA CGTGAGCTAG CTGGCATGGC GGCCTGCATT | 37 |
| | | GCAGCGGGGC ACTGGGCTGC AATGGGCCTA GGCCGGAGTT | |
| | | TCCAAGCCGC CAGGACTCTG CTCCCCCCGC CGGCCTCTAT | |
| | | CGCCTGCAGG GTCCACGCGG GGCCTGTCCG GCAGCAGAGC | |
| | | ACTGGGCCTT CCGAGCCCGG TGCGTTCCAA CCGCCGCCGA | |
| | | AACCGGTCAT CGTGGACAAG CACCGCCCCG TGGAACCGGA | |
| | | ACGCAGGTTC TTGAGTCCTG AATTCATTCC TCGAAGGGGA | |
| | | AGAACAGATC CTCTGAAATT TCAAATAGAA AGAAAAGATA | |
| | | TGTTAGAAAG GAGAAAAGTA CTCCACATTC CAGAGTTCTA | |
| | | TGTTGGAAGT ATTCTTCGTG TTACTACAGC TGACCCATAT | |
| | | GCCAGTGGAA AAATCAGCCA GTTTCTGGGG ATTTGCATTC | |
| | | AGAGATCAGG AAGAGGACTT GGAGCTACTT TCATCCTTAG | |
| | | GAATGTTATC GAAGGACAAG GTGTCGAGAT TTGCTTTGAA | |
| | | CTTTATAATC CTCGGGTCCA GGAGATTCAG GTGGTCAAAT | |
| | | TAGAGAAACG GCTGGATGAT AGCTTGCTAT ACTTACGAGA | |
| | | TGCCCTTCCT GAATATAGCA CTTTTGATGT GAATATGAAG | |
| | | CCAGTAGTAC AAGAGCCTAA CCAAAAAGTT CCTGTTAATG | |
| | | AGCTGAAAGT AAAAATGAAG CCTAAGCCCT GGTCTAAACG | |
| | | CTGGGAACGT CCAAATTTTA ATATTAAAGG AATCAGATTT | |
| | | GATCTTTGTT TAACTGAACA GCAAATGAAA GAAGCTCAGA | |
| | | AGTGGAATCA GCCATGGCTT GAATTTGATA TGATGAGGGA | |
| | | ATATGATACT TCAAAAATTG AAGCTGCAAT ATGGAAGGAA | |
| | | ATTGAAGCGT CGAAAAGGTC TTGATTCTGA GAATGAATTT | |
| | | GGTTAGTTGC AGAAGATACA TTGGCTCTAA GAGGATATAT | |
| | | TTTGAGACCA ATTTAATTTC ATTTATAAGA ACATAGTAAT | |
| | | TAAGTGAACT AAGCATTCAT TGTTTTATTA ATACTTTTTT | |
| | | TCTAAAATAA AACTTGTACA CCAGTTTATT ACTCTAAAAA | |
| | | GAGAATTACA CATGCCAAAT GGACCAATGT CCATTTGCTT | |
| | | ATTGGAGGCA AAGCTACAAT AGAAGTCAGA GCATCACCAG | |
| | | AATGGTCTTT AATGAGCATG GAACCTGAGC AAAGGGAATA | |
| | | GGTGGGATGA ATTTTTTTTT TAATTGTGAA ACAATTCATA | |
| | | AGCACAATAT GATTTACAGA ATAATAAACA TTCATGTACC | |
| | | CACTATCAGG TTAAGAAATA GAACATTTAT TAATATGTAG | |
| | | GAATGTTAAG AAATAAAACA TTTAATAAGA TCTCAGAAGA | |
| | | CTCCAGTAAA TCTGCAATTG TATCTCTCTC CTTTTTAAAT | |
| | | GTAAATATCA TCTTGACTTG TTAATTATTC CCTTGCATTT | |
| | | CTTTTAGTTT ACTGCCAACA CATATATTCT TCAACAATAT | |
| | | ATTTAATTTT GAAAAACCTG AAAAAAAAAA CCTGTTAGCA | |
| | | AGTATAAAGG GGCAGTATTA CTATTATTGC ATGAAGGCTT | |
| | | CAAGGGAAAC GTTACAGTCT TTGGGTCATA GTCTGGCTTC | |
| | | AGCTTCCTCT GAGAGTTTAC AGAGGCCAAT TTTGAGCAAA | |
| | | TTCATGGCTA AGGTTATGAG TGAGTTCTGC TAAACAGAAG | |
| | | GCTCACCACA AGGTATCTGG CAGGATTATA CTGGGTAGCT | |
| | | GGATGTTGCA GAAATGTGGT TAGAGGAAGT AAACTGTTTT | |
| | | TTGATGCTCA CAGCATGATG AATCAAACTC TGTATCTTAG | |
| | | GATTAGGTTA AAACAATACC TTTGGTATGA TATGAGTGTT | |
| | | GTTGCTGATC CATGCAGCAT GGATTGGAAA GCTGGGGTAT | |
| | | AAGCACACAT GCTAAAGAAA AACATGTAAT TTGGTCCATA | |
| | | CTCACCTGGA TATACTGTTC CTCAGGTTAA AAAATACAGT | |
| | | ACTATCCTAA ATCTTGAAGG CAACTCTCAG CCTATCCATT | |
| | | GAGTTACCTT CAGATCTGCC CTCTGGTTCC TAGCTGTCTT | |
| | | GGGACTAACT TCTTTCCTGC GCTCAGCTGT TTTCTGGATT | |
| | | CCATGTTTTC CATTTTATTG AGTACTAACT TGTTTTGCTG | |
| | | CAGCACATCC TTTGGTAGCT TCTAGAGGAA GTTTGTGTGG | |
| | | AGGTAAAATT TTTGAGACCT TGCATGTCTC ATGTTTGATT | |
| | | GATACTTTAT ACGTTTAGGT AGGAGGTAAT TTTCCTTCAG | |
| | | GACTTTAAAA ATATTGTTGC TCCATTTTCT TTGTTTCTAT | |
| | | TGTTGTATTG AGAAATCCAA TGCCATTTTG ATTTCCCCAT | |
| | | CATAAATTTC ATGATGATGT GTCTTGGTGT GGGTCTATAT | |
| | | TTATCCATTG TATTGGGTTT TAGGTGAACC CTTCCAGATA | |
| | | GTAACTCATT TCTGTCAGTT CTGGGAAACA CTTAGCATTG | |
| | | GTTGATGATT TATTCTCTGC TGCTTTGTTC TCCCAACTAT | |
| | | TATTTGGATG TTGGATATCC AGCACTGGGT ATCTATTTTC | |
| | | TTACCTCCCT CCCTTGACCC CAGTCTCTGT TTTTTAGCTC | |
| | | TTTAGCTCAA TCTTCCAACT CTTTGCTATT GTATTTTAAA | |
| | | ATCTTAAGAC CCCTTCTTGA TTTGTAGAAG TTCCTTTTCT | |
| | | TACAACCAAA AAGCCTTTAT CTATGGATTT GTTCACAGAT | |
| | | AAGGGGTATT CAATATAGTG TATTTTTTTT TCATTTAAAA | |
| | | TTGTTTGCGC ATCTATTTCC TCCAAATTTC TTTCTGTATT | |
| | | TATTTTTTGT TGTCTATATT TCAGACTTTT CCAGGATATC | |
| | | TGATAATCTT TGGCTGTCTT CTTATGGTTG AAAGAGGGAC | |
| | | TAAAAAGCTT GGAAAGCCTT TGGGTTGTGG GAAGGGGCTG | |
| | | TCTTTAGGAT TATCTGAATG GGCTTTTTTG GGAGTCCCCT | |
| | | CCTCCACATG AATATTTTGG TTTTGTCAGA TTCCCTAGAA | |
| | | TAGAGGCTTC CAATCTCCTT CCTGGAGGGG TCTGTCCAGG | |
| | | AAGGAGATTG TCTAGGGGTC TGTCAGACAG CAGCTTTCAG | |
| | | CTACTTCCTT GATCTTTTTC ACTAATGATT ATATAGTCAT | |
| | | CTAACTACTG TCAACAAGTA ATAGATATCC TATCCTTCAC | |
| | | TTGTTTAGAT TATTTGCTGA GATAACCTCT CAAAAGAACC | |
| | | TCTCAAAATA AAAGGTTAAC AAGAGCCTAT ATCTTATATT | |
| | | TTTCTTCTCT TTATCTTGTT AGAAGATAGC TATTAAAACC | |
| | | TGTTCTTTTT CTGTCTTGAT AAACACACTT CAATCTTGGT | |
| | | AGAATGGTAG ATGGGACAGT ATATTTTAGG ACCTAAAGCT | |
| | | CTGCAAATGT ATGATCAGCT TGTAAGTACA GGTGCTCAAA | |
| | | AACATGTAAA CAATCATGCT TTTTACTCTG TAGGAATATC | |
| | | TTTAAAATTC TTGTGAATTT TTCCCCAGAA GTAAAGCAAA | |
| | | TCTTCCCCCA GAAATAAAAT TAAATGTGCA TAATCTAAAG | |
| | | CTTTTTTTTT TTATTGTGGT AGGATATATA TATAAAACAT | |
| | | AATTTGCCAT TGTAAACATT TTAAATTTAC AAGTCAGAGG | |
| | | CATTAATTAC ATCACAATGT TGTGAAATTA TTACTACTAT | |
| | | TTCCAAAATT TTCTCATCAC CCCAAACTGA AACTCTGTAA | |
| | | CTGTTGAGCA ATAACCTCAT TCCTGTATCT CTCCCAACCC | |
| | | CAGGTAACCT CAAATCTTTC TTTTTATCTT TGAGACAAGG | |
| | | TCTCATTCTA TCACTCAGGT AGGAGTGCAG TGGTGTGATC | |
| | | ATAGCTCATT GCAGCCTCAA AATCCTGGGC TCAAGCAATC | |
| | | CTCCTTGAGT AGCTAAGACT ATAGGCACAC ATTAACTGCG | |
| | | CCTGGCTGAT TTTGTTTTTT GTAGAGATGT GGTCTTGCTA | |
| | | TGTTTCCCAT GCTGGTCTTG AGTTCCTGGC CTCAAGCAGT | |
| | | CCTTAAGATT CATCCATGTT GTGGCATGTG TCAGAATTTC | |
| | | ATTTGTTTTT ATGACTAAAT AATATTCCAT TGTATGTATA | |
| | | TACATTTTGT TCATCCATCT TCTGATGAAC ACTGGGATAT | |
| | | GTCTACCTTT TGGCTATTGT GAATAATGCT GCAGTAAACA | |
| | | TTGACATAAC AAGTATGTAT TTGATTGCCT GTTTCTAAGT | |
| | | TCTTTTGGGT ATACATCTTG AGTAGAATTG CTAGATAATG | |
| | | TCATGTTTTA TTTCTCTTGT GATTTCTTCT TCGATCCCCT | |
| | | GGTTGAGTGT GTTAATTTCT ACATGTTTAT GAATTTCCCA | |
| | | CTGTTTTTTT GTTATTGATT TCCAAGTTCA TTCCATTGTG | |
| | | ATTAGAGAAG ATACTTAGTA TGATTTTAAT GTTTTTGAGA | |
| | | ATTGGTGTGT GGCCTGATAG ATGGTCTGTC CTGGAGAATG | |
| | | TTCCTCATAC ACTTGAGCAA AATATTTATC ATGCTATTGT | |
| | | TGACTGTAGT TTTCTATATG TCTCTTAGGT CAAGGTGGTT | |
| | | TACAATGTGT TAAGGTTCTC TTTTTTTAAA AAAATTTTTG | |
| | | CACAGAGTAT CTTTTTCTAT GTGTTCCATG TATTTGTGTC | |
| | | TTTGGAGCTA TAGTCTCTTG TAGACAGCAT ATCACTATCT | |
| | | TGTTTTGTTT TGTTTTTTCT GTCCATTCTG CCAATTTCTG | |
| | | CCTTTTGATT GGAAAATTTA ATCCATTTGC ATTTAAAGTA | |
| | | ATTAAGGAAG GACTTTCTTC TACCATTTAA CACTTCTTCT | |
| | | ATATGTCATA TACTTTTTTG GCCCCTCATT TCCTCTTTAT | |
| | | GGCCTTCTTT TCTGTTTTTT TGTAGTGAAC TAGTCTGATT | |
| | | CTCTTTCCAC TCCCCTTTGT GTATATTTGT TAGATGTTTT | |
| | | ATTTGTGGTT GCTATGGGGA TTATAGTTAA CATCCTACAC | |
| | | TTAAAACAAT CTAATTTAAA CTGATACCAA TTTACCTTCA | |
| | | ATAGCATACA AAATCTCTAC TCCTGTAAAG CTCTGCCCCT | |
| | | GCCCCCCTTA TGTTATTGAT GGCACAAATT GCCTAATAAA | |
| | | TAATTTATAG TTATTTGTAT GAGTTTGTCT TTTAAATCAT | |
| | | TTAGGAAATA AAAAGTGGAG TTAGAAAACA GTATGATAGT | |
| | | AATACTGACT TTTATATTTG TCAATATATT TATCTTATTT | |
| | | TGGATCCTTA TTTCATTATA TAGATTTGAG TTACTGTCTA | |
| | | GTGCCCTTCC ATTTCGGCCC AAAGGATTCC CTTATGCATT | |
| | | TCTTGCAGGG CAAGTCTAAT TGTAATAAAC TCCCTCAGCT | |
| | | TTTGTTTTAT CTGAGAATGT CTTGATTTCT CCCTTATTTT | |
| | | TGATGGATAA TTTTGCCAGA TACATGAATT TTTGGTAACA | |
| | | GTATTTTTCT TTCAGCACTT TAAATATGTC ATCCCACTAC | |
| | | CTTCTGACTT CATGGTTTCT CATGAGATAT TAGATGTTAT | |
| | | AAAATTTGAG GATTCCTCAT TCTTGATGAG TCAGTTCTGT | |
| | | CTTATTGCTT TTCGGATTTG CTCAGCTTTT GTCTTTTGAC | |
| | | AGTTTGATTA TAACGCGGCT CAGTGTGGGT CTCTGAGTTT | |
| | | ATCCCACTTA GAGTTTGTTG AGTTTCTTGG AGTCATAGAT | |
| | | TTATGTCTTT TATCAAATTT TGGACATATT TGGCTATTAT | |
| | | TTCTTCAATT TTTTTCACTG CTTCTTTCTT TTCCTTCTGA | |
| | | AATATTCTTA ATGTATATGT TGGTCTGTTT GATGCTGTCT | |
| | | CACCAGTTTC TTAGGCTGTG TTCTCTTTTG TTCCTCAGAC | |
| | | TTGATTATTG CAGTTGCCCT TCTTTTTATT TTTTTCAAGT | |
| | | TTGTTGATTC TTCTCCCTGT TCAGATCAAC TGTTGAACTC | |
| | | CTCTAGTGAA TTTATTTCAG TTACTGTACT TTTCAGCTCC | |
| | | AAGATTTATC TTTGGTTCCT TTTTATAACG TCTGTGTCTT | |
| | | TATTGATATT CTCATTTTGT TCATATGTCT CTTTCTTCCT | |
| | | TTAGTTCTTT GTCCATGTTT TCCTTTAGCT CTTTGGGCTT | |
| | | ATTTAAGACA ATTGTTTAAA GTCTTTGCAT AGTAAGTCCA | |
| | | ATGTCTGTGT TTCTTCAGGG ATGGTTTTCA TTATTTTGTT | |
| | | TTCAATGAGC CATACTTTCC TGTGTCTTTG TATGCTGTCT | |
| | | TTTTGTTGTT GAAAACTGTA TGTTTGAACA TCATAACGTG | |
| | | GTGGCCCTGA AAATCAGATA TTCCCCCCTT CCTGAGAGTT | |
| | | AGTTTTATTT TTATTATTGA AGATTGTAGC AGTCTATTGC | |
| | | TACATGTGCA GTCATTTCCA AACTATTTTT GCAAAGACTG | |
| | | TATTCCTTCT GTGTGTCATC ACTGAAGTCT CTGTTCCTTA | |
| | | GTTTGTGTTT AATAGTTTGA CATAGATTTC CTTGAAAGGA | |
| | | GTTAAAACTA GCAGAAAAAT CTCTCTCCCA GTCTTTCCAG | |
| | | TCTTTGTAGA TTGGTTCTGT GCTGGGCTTT TCCATTAATA | |
| | | CTTAGCCAGG CTTGTACTGA GCCTAACAAT CAGGCCCAAA | |
| | | AGCGTAGGGT CTTTGCAGAT CTTGTCTGAG CATGCTTCTT | |
| | | GCTGTGTATG CACGTAGTTT TCTAAATCTC CCTGTATGTG | |
| | | CTGTTGAATA TTCTAATTTC CCAAAGAAAC TCCTTTGCAG | |
| | | CTTTTTCTCA CAGAACATAG ATGGTTTTTT GGATATCTTG | |
| | | ACCATAGTCT TTCGACCCAG GTGTTTGCGG TTGTTAGTTC | |
| | | ACCTTACACT TTTTTCAAGC ATTGCCTACT GCTTACGATG | |
| | | AGTGCTCTGT CAATCCTTTA AGTAGCCCCA GACAGGCTAC | |
| | | CAGAGACTTA AACAAGAATT TGTAAGTTCT GCTCAGCTTC | |
| | | CTCTAGAAAT GGGGATCAGG GTCCAAGACA GAATGCAGTT | |
| | | GCTGATTTCA AGACTGCTGC AACACCAGGG AGCTTGTGGG | |
| | | GGAAGGGCAA GCAGAAATGT CACAAAGCTT TCTTGCCATT | |
| | | TTAAAGTTGC CTGTTCTTGA CTCAGCATTT GCTTCATTGC | |
| | | TATAAACTTT TTACTGTTTT TCAGAGTTCT GATAAAATTG | |
| | | GCTATGCCTG TTCCTGCTTT AAAAAATATA TATATATTTT | |
| | | TTAGGGATTG GGGTCTCACT ATACTGACCA GGCTGGTCTT | |
| | | GAACTTCTGG CCTCAAGCCA TCCTCTCATT TCAGCTTCCC | |
| | | AAAGTGCTGC AATTACACGC GTGAACCACC ACACCCAGCC | |
| | | CCTGCTTGTT TTTCAATGTG CCTACTCCAC CATGTTGCTC | |
| | | AAGTATGTAT ATTTTCTAAA CTACCTTGTA GTGTTGTGAT | |
| | | GGGAAATAAA TCCCTGAGCC TTTTGAATAA CTCAGAGAGA | |
| | | TCAAAAACTT AGTTTATCCT ATTCGAAGGA TTAGAAAAAT | |
| | | GATATATCTT TCACTTTTTC AGGGATAGGC TCCTCATTAG | |
| | | AAGGCTCCTA TGTGCCGATG CTGTACAAGA CATTTCATTT | |
| | | CTCTTAATGT TTACAACAAG CTTGTTGCCA AGGCTGATCT | |
| | | TGAACTCCTG GCCTCAAACG ATCCTCCCAG CTCAGTCTCA | |
| | | CAAAGTGTTG GGATGTCTGG CCAACTAATG ACTATCTTAA | |
| | | CTCTTGTGTT TCAATGTTTA TGCCTTCTTT TATCTTGACT | |
| | | GATTGTATGA CTATGTCTTC TAGAACAATG TTGAACAGAA | |
| | | ATGGTGAGAG CAGACATCCT TGCTTTAATA TTTCACCATT | |
| | | ATATATGATG TTAGGTATAG ATTTTTCTCA CAGATGCCTT | |
| | | TTATCAGATT GAGGAATTTA TATTCCTACT TTGCCGAAAG | |
| | | GTTTTTGTAG TATGAGGGGG TGCTGAATTT TGTCAAACAC | |
| | | TTTTTCGGTA ATAATTGAGA TGATTGGTTC TGCAGTCATC | |
| | | GAGATGTGGA TTTTCTCCTT TATTCTGTTC GTGAGTGATT | |
| | | ACACTGGTTG ACTAATGTTA AAACAACCTT ACTTTCCAGG | |
| | | AATAAACCCT ATTATCTTTT TTATACA | |
| PSMC4 | NM_153001.2 | TGCGGGTACG GACAGCGCAT GAGCTTATGT TGAGGGCGGA | 38 |
| | | GCCCAGACCA GCCCTTCGTC CTATCCTGCC CTTCCAGCAC | |
| | | CTCTCAGCCG TAACTTAAAC TACACTTCCC AGAAGCCTCC | |
| | | TCAGCCAGGG ACTTCCGTTG TCGTCAGCGG AAGCGGTGAC | |
| | | AGATCATCCC AGGCCACACA GAGGCCGGCT TGGTCACTAT | |
| | | GGAGGAGATA GGCATCTTGG TGGAGAAGGC TCAGGATGAG | |
| | | ATCCCAGCAC TGTCCGTGTC CCGGCCCCAG ACCGGCCTGT | |
| | | CCTTCCTGGG CCCTGAGCCT GAGGACCTGG AGGACCTGTA | |
| | | CAGCCGCTAC AAGGAGGAGG TGAAGCGAAT CCAAAGCATC | |
| | | CCGCTGGTCA TCGGACAATT TCTGGAGGCT GTGGATCAGA | |
| | | ATACAGCCAT CGTGGGCTCT ACCACAGGCT CCAACTATTA | |
| | | TGTGCGCATC CTGAGCACCA TCGATCGGGA GCTGCTCAAG | |
| | | CCCAACGCCT CAGTGGCCCT CCACAAGCAC AGCAATGCAC | |
| | | TGGTGGACGT GCTGCCCCCC GAAGCCGACA GCAGCATCAT | |
| | | GATGCTCACC TCAGACCAGA AGCCAGATGT GATGTACGCG | |
| | | GACATCGGAG GCATGGACAT CCAGAAGCAG GAGGTGCGGG | |
| | | AGGCCGTGGA GCTCCCGCTC ACGCATTTCG AGCTCTACAA | |
| | | GCAGATCGGC ATCGATCCCC CCCGAGGCGT CCTCATGTAT | |
| | | GGCCCACCTG GCTGTGGGAA GACCATGTTG GCAAAGGCGG | |
| | | TGGCACATCA CACAACAGCT GCATTCATCC GGGTCGTGGG | |
| | | CTCGGAGTTT GTACAGAAGT ATCTGGGTGA GGGCCCCCGC | |
| | | ATGGTCCGGG ATGTGTTCCG CCTGGCCAAG GAGAATGCAC | |
| | | CTGCCATCAT CTTCATAGAC GAGATTGATG CCATCGCCAC | |
| | | CAAGAGATTC GATGCTCAGA CAGGGGCCGA CAGGGAGGTT | |
| | | CAGAGGATCC TGCTGGAGCT GCTGAATCAG ATGGATGGAT | |
| | | TTGATCAGAA TGTCAATGTC AAGGTAATCA TGGCCACAAA | |
| | | CAGAGCAGAC ACCCTGGATC CGGCCCTGCT ACGGCCAGGA | |
| | | CGGCTGGACC GTAAAATTGA ATTTCCACTT CCTGACCGCC | |
| | | GCCAGAAGAG ATTGATTTTC TCCACTATCA CTAGCAAGAT | |
| | | GAACCTCTCT GAGGAGGTTG ACTTGGAAGA CTATGTGGCC | |
| | | CGGCCAGATA AGATTTCAGG AGCTGATATT AACTCCATCT | |
| | | GTCAGGAGAG TGGAATGTTG GCTGTCCGTG AAAACCGCTA | |
| | | CATTGTCCTG GCCAAGGACT TCGAGAAAGC ATACAAGACT | |
| | | GTCATCAAGA AGGACGAGCA GGAGCATGAG TTTTACAAGT | |
| | | GACCCTTCCC TTCCCTCCAC CACACCACTC AGGGGCTGGG | |
| | | GCTTCTCTCG CACCCCCAGC ACCTCTGTCC CAAAACCTCA | |
| | | TTCCCTTTTT TCTTTACCCA GGATTGGTTT CTTCAATAAA | |
| | | TAGATAAGAT CGAATCCATT TAATTTCTTC TTAGAAGTTT | |
| | | AACTCCTTTG GAGAATGTGG GCCTTGAATA GGATCCTCTG | |
| | | GGTCCCTCTT AATCTGACAG ATGAGCAGAC GAGGTGCATG | |
| | | GCCTGGGTTG CAGCTTGAGA GAACCAAAAT ATTCAAACCA | |
| | | GATGACTTCC AAAATGTGGG GAAAGGGATG GAAAATGAAC | |
| | | CTGAGATGGA GTCCTTAATC ACGGGATAAA GCCCTGTGCA | |
| | | TCTCCCTCAT TTCCTACAGG TAAAAGACAG TAAAGAAATT | |
| | | CAGGTCACAG GCCTTGGGAG TTCATAGGAA GGAGATGTCC | |
| | | AGTGCTGTCC AGTAGAACTT T | |
| SF3A1 | NM_005877.5 | GGTCCCGGAA GTGCGCCAGT CGTACCTTCG CGGCCGCAAC | 39 |
| | | TCGCTCGGCC GCCGCCATCT TGCGAGCTCG TCGTACTGAC | |
| | | CGAGCGGGGA GGCTGTCTTG AGGCGGCACC GCTCACCGAC | |
| | | ACCGAGGCGG ACTGGCAGCC CTGAGCGTCG CAGTCATGCC | |
| | | GGCCGGACCC GTGCAGGCGG TGCCCCCGCC GCCGCCCGTG | |
| | | CCCACGGAGC CCAAACAGCC CACAGAAGAA GAAGCATCTT | |
| | | CAAAGGAGGA TTCTGCACCT TCTAAGCCAG TTGTGGGGAT | |
| | | TATTTACCCT CCTCCAGAGG TCAGAAATAT TGTTGACAAG | |
| | | ACTGCCAGCT TTGTGGCCAG AAACGGGCCT GAATTTGAAG | |
| | | CTAGGATCCG ACAGAACGAG ATCAACAACC CCAAGTTCAA | |
| | | CTTTCTGAAC CCCAATGACC CTTACCATGC CTACTACCGC | |
| | | CACAAGGTCA GCGAGTTCAA GGAAGGGAAG GCTCAGGAGC | |
| | | CGTCCGCCGC CATCCCCAAG GTCATGCAGC AGCAGCAGCA | |
| | | GACCACCCAG CAGCAGCTGC CCCAGAAGGT CCAAGCCCAA | |
| | | GTAATCCAAG AGACCATCGT GCCCAAAGAG CCTCCTCCTG | |
| | | AGTTTGAGTT CATTGCTGAT CCTCCCTCTA TCTCAGCCTT | |
| | | CGACTTGGAT GTGGTGAAGC TGACGGCTCA GTTTGTGGCC | |
| | | AGGAATGGGC GCCAGTTTCT GACCCAGCTG ATGCAGAAAG | |
| | | AGCAGCGCAA CTACCAGTTT GACTTTCTCC GCCCACAGCA | |
| | | CAGCCTCTTC AACTACTTCA CGAAGCTAGT GGAACAGTAC | |
| | | ACCAAGATCT TGATTCCACC CAAAGGTTTA TTTTCAAAGC | |
| | | TCAAGAAAGA GGCTGAAAAC CCCCGAGAAG TTTTGGATCA | |
| | | GGTGTGTTAC CGAGTGGAAT GGGCCAAATT CCAGGAACGT | |
| | | GAGAGGAAGA AGGAAGAAGA GGAGAAGGAG AAGGAGCGGG | |
| | | TGGCCTATGC TCAGATCGAC TGGCATGATT TTGTGGTGGT | |
| | | GGAAACAGTG GACTTCCAAC CCAATGAGCA AGGGAACTTC | |
| | | CCTCCCCCCA CCACGCCAGA GGAGCTGGGG GCCCGAATCC | |
| | | TCATTCAGGA GCGCTATGAA AAGTTTGGGG AGAGTGAGGA | |
| | | AGTTGAGATG GAGGTCGAGT CTGATGAGGA GGATGACAAA | |
| | | CAGGAGAAGG CGGAGGAGCC TCCTTCCCAG CTGGACCAGG | |
| | | ACACCCAAGT ACAAGATATG GATGAGGGTT CAGATGATGA | |
| | | AGAAGAAGGG CAGAAAGTGC CCCCACCCCC AGAGACACCC | |
| | | ATGCCTCCAC CTCTGCCCCC AACTCCAGAC CAAGTCATTG | |
| | | TCCGCAAGGA TTATGATCCC AAAGCCTCCA AGCCCTTGCC | |
| | | TCCAGCCCCT GCTCCAGATG AGTATCTTGT GTCCCCCATT | |
| | | ACTGGGGAGA AGATCCCCGC CAGCAAAATG CAGGAACACA | |
| | | TGCGCATTGG ACTTCTTGAC CCTCGCTGGC TGGAGCAGCG | |
| | | GGATCGCTCC ATCCGTGAGA AGCAGAGCGA TGATGAGGTG | |
| | | TACGCACCAG GTCTGGATAT TGAGAGCAGC TTGAAGCAGT | |
| | | TGGCTGAGCG GCGTACTGAC ATCTTCGGTG TAGAGGAAAC | |
| | | AGCCATTGGT AAGAAGATCG GTGAGGAGGA GATCCAGAAG | |
| | | CCAGAGGAAA AGGTGACCTG GGATGGCCAC TCAGGCAGCA | |
| | | TGGCCCGGAC CCAGCAGGCT GCCCAGGCCA ACATCACCCT | |
| | | CCAGGAGCAG ATTGAGGCCA TTCACAAGGC CAAAGGCCTG | |
| | | GTGCCAGAGG ATGACACTAA AGAGAAGATT GGCCCCAGCA | |
| | | AGCCCAATGA AATCCCTCAA CAGCCACCGC CACCATCTTC | |
| | | AGCCACCAAC ATCCCCAGCT CGGCTCCACC CATCACTTCA | |
| | | GTGCCCCGAC CACCCACAAT GCCACCTCCA GTTCGTACTA | |
| | | CAGTTGTCTC CGCAGTACCC GTCATGCCCC GGCCCCCAAT | |
| | | GGCATCTGTG GTCCGGCTGC CCCCAGGCTC AGTGATCGCC | |
| | | CCCATGCCGC CCATCATCCA CGCGCCCAGA ATCAACGTGG | |
| | | TGCCCATGCC TCCCTCGGCC CCTCCTATTA TGGCCCCCCG | |
| | | CCCACCCCCC ATGATTGTGC CAACAGCCTT TGTGCCTGCT | |
| | | CCACCTGTGG CACCTGTCCC AGCTCCAGCC CCAATGCCCC | |
| | | CTGTGCATCC CCCACCTCCC ATGGAAGATG AGCCCACCTC | |
| | | CAAAAAACTG AAGACAGAGG ACAGCCTCAT GCCAGAGGAG | |
| | | GAGTTCCTGC GCAGAAACAA GGGTCCAGTG TCCATCAAAG | |
| | | TCCAGGTGCC CAACATGCAG GATAAGACGG AATGGAAACT | |
| | | GAATGGGCAG GTGCTGGTCT TCACCCTCCC ACTCACGGAC | |
| | | CAGGTCTCTG TCATTAAGGT GAAGATTCAT GAAGCCACAG | |
| | | GCATGCCTGC AGGGAAACAG AAGCTACAGT ATGAGGGTAT | |
| | | CTTCATCAAA GATTCCAACT CACTGGCTTA CTACAACATG | |
| | | GCCAATGGCG CAGTCATCCA CCTGGCCCTC AAGGAGAGAG | |
| | | GCGGGAGGAA GAAGTAGACA AGAGGAACCT GCTGTCAAGT | |
| | | CCCTGCCATT TTGCCTCTCC TGTCTCCCAC CCCCTGCCCC | |
| | | AGACCCAGGA GCCCCCCTGA GGCTTTGCCT TGCCTGCATA | |
| | | TTTGTTTCGC TCTTACTCAG TTTGGGAATT CAAATTGTCC | |
| | | TGCAGAGGTT CATTCCCCTG ACCCTTTCCC CACATTGGTA | |
| | | AGAGTAGCTG GGTTTTCTAA GCCACTCTCT GGAATCTCTT | |
| | | TGTGTTAGGG TCTCGATTTG AGGACATTCA TTTCTTCAGC | |
| | | AGCCCATTAG CAACTGAGAG CCCAGGGATG TCCTACAGGA | |
| | | TAGTTTCATA GTGACAGGTG GCACTTGGCT AATAGAATAT | |
| | | GGCTGATATT GTCATTAATC ATTTTGTACC TTGACATGGG | |
| | | TTGTCTAATA AAACTCGGAC CCTTCTTGTG AAATCAGTTA | |
| | | AATAAGACTT GTCTCGGTCA CCTGTGCCCT GTCCAGACTC | |
| | | GAGGCAGTGG TAACACTGCA CAGTGCTATG TGGCTTCTCT | |
| | | TTGAGGATTT TTGGGTTTTG TAACTAAATT CTTGCTGCCC | |
| | | TCATACTTTT TATGTATTAG AATCATATTC GTATTGCCCT | |
| | | TTTAAAACAT TGGGATCCTC CAAAGGCCTG CCCCATGTAT | |
| | | TTAACAGTAA TACAGGAAGC ATGGCAGGCA CCATGCAAAC | |
| | | CAAGGATGGA TGGTGCAGTC CCTGTGTCAG TGGGCGGTGG | |
| | | TTTCCTGCTG GCCTGGAATC ACTCATCACC TGATTGATTG | |
| | | GCTCTGTGGT CCTGGGCAGG TGCCTCATAG GTGTGTGGAT | |
| | | ATGATGACGT TTCTTTAAAA TGTATGTATT TAACAAATAC | |
| | | TTAATTGTAT TAAGGTCATG TACCAAGGAT TTGATAAAGT | |
| | | TTAAATAATT TACTCTCTAC TTTTATCCAT TTTATCCATT | |
| | | TTAACTCATG TAATCCTCAT GTGAGTATTC CTGTTTAACA | |
| | | CTTGAGTAAA CTGAGGCACA GAGAACATAA GTTGCATGCC | |
| | | ATAGTCACAC ACTGTGAAAG TGAAAAGAGA ATGTGTGCAA | |
| | | AACACGTCAC AGTCCTGGTT TCTGAGTAAA GGCAGGCTGT | |
| | | TATCTTTAGA ATCAAGCTAT CACAGGGAGA TAGGCAATGC | |
| | | TGTGGGTGTT GGAGGAAGGT GAGAGCCTGT TGCTAACAAT | |
| | | TTCCTGGTTT TAAAGCTAAG GCTGATTTTA TTGGGAAGAT | |
| | | CTCACATGTG TGTGGCCCCT GAGAGTTCCC AGTGCCTTTT | |
| | | ATTTGCAGTC CTTCCATTTG GACCTCCTAG CTGCCCCATC | |
| | | AGGTCATCTC CAGGGCTCAG AGGGGTGAGA CCATTTCCCA | |
| | | AGGTCACAGA ACCAGCTCTC TAGTCACCAC CCTGCCTCTC | |
| | | CCTCTCACCC AGAGTCAGTA CCAGTTTTAT GGCTTTATTA | |
| | | CAAACTGCTG GGTCCCTCCC ATTTTCAACT TGATTGATGG | |
| | | GATGTCATCC CTTATCCTGT CTGACATTTG CCTCTGGCCT | |
| | | GGTTGCTAGA AGTTTGCCCC AGGGGCAAGA GTTGAAATTT | |
| | | GGCTTCCTGA GGTGGGCTTT GTGGTTTGCG TCCCTAAAGT | |
| | | GAGCCCACTA CTGGTTGCTT GTCCATGGCC AACACCAGAA | |
| | | ATCCCCTGAG CACTACCTGG GTCTCATTCC AAGAAGGAAG | |
| | | AGGGTCAGGA GACCTGGGGA GTCTCATATT CCAAGTTCTT | |
| | | CTTTCTTTCT GGGAGCAGTG GGCAGTTCAT GGTGTTAGGG | |
| | | CACTCACCCC CACAGACTGG CAAACCCTGC AGGACTTCCG | |
| | | TGGCTGAGGC TGTGACCGGA GGCCAGGAAT GCCGTTGGGT | |
| | | GGATTGTGAG TGAATGGGCC CTTTGAGCTG CCCTCTAGAG | |
| | | AGCAAATCCA GTTTCCTGGA GCTCCTGAAT GAATATCTGT | |
| | | ACTGGCTCGC TCAGATGCAG AAGCTCCATT GACCATGAGG | |
| | | CCTTGTGAAC ATCAGTGGCC ACAGGCCCAG TGTGCTGCTT | |
| | | GGCACTGCAC TAGTTTAGGA CCTGCAGCAT GTAGGTAGCG | |
| | | TCCTAGTGTT TATAATACAA AGCTGCTCTG CACAGCTTTT | |
| | | CTGATTCTTC TTGCAATCTC CTGAGGATTA TCTGCCCCAT | |
| | | TTTTAAAACG AGGTGGAATA CCCAAGGTCA TGTAGCCAGT | |
| | | GAGTGCTCTG GAAAGCCAAA GCAGCTCATC CCTTCCTGGG | |
| | | GACCACACTG CTCTGCTCCA CCAGACCACA CTATGAAATA | |
| | | GGAATAAGTG CTCCTGTTGC AGGACTGCTG GGAAAACAGG | |
| | | TGGTGTGGGA CTTAAGTCAC CATAATTTTG AAGACTTGCA | |
| | | TGCAGAGGGC TCCAGGAATT GTAGACATTA AGGAATTTCA | |
| | | CTTTCAGTTC TACCCACTAC TTAAGTACTT GTCATGTACT | |
| | | CTTAGAGGAG GCCAGTAATG ATCAGAACCA TTTTACTTTA | |
| | | AAATTAATAA TATTGTATTA GAGAATATAT TAAATGGTTA | |
| | | TATTGGGTTA TGTTAGGATA TATACTTGAA TGGAAATACA | |
| | | TGTACTATTA GCAATCATAT TTCATTTATC CCTGTAATTA | |
| | | GACAAGAAAG CATAATATAG CTCTACTCAT GGGTACACAT | |
| | | ACCAGTGTAT AAGATTTTTA GAAGTTTACT TTTTAAAAAT | |
| | | AAAAGCAAAA TGTAAGATCT TAAAAAAAAA AAAAAAAAA | |
| PUM1 | NM_001020658.1 | AGTGGGCCGC CATGTTGTCG GAGTGAAAGG TAAGGGGGAG | 40 |
| | | CGAGAGCGCC AGAGAGAGAA GATCGGGGGG CTGAAATCCA | |
| | | TCTTCATCCT ACCGCTCCGC CCGTGTTGGT GGAATGAGCG | |
| | | TTGCATGTGT CTTGAAGAGA AAAGCAGTGC TTTGGCAGGA | |
| | | CTCTTTCAGC CCCCACCTGA AACATCACCC TCAAGAACCA | |
| | | GCTAATCCCA ACATGCCTGT TGTTTTGACA TCTGGAACAG | |
| | | GGTCGCAAGC GCAGCCACAA CCAGCTGCAA ATCAGGCTCT | |
| | | TGCAGCTGGG ACTCACTCCA GCCCTGTCCC AGGATCTATA | |
| | | GGAGTTGCAG GCCGTTCCCA GGACGACGCT ATGGTGGACT | |
| | | ACTTCTTTCA GAGGCAGCAT GGTGAGCAGC TTGGGGGAGG | |
| | | AGGAAGTGGA GGAGGCGGCT ATAATAATAG CAAACATCGA | |
| | | TGGCCTACTG GGGATAACAT TCATGCAGAA CATCAGGTGC | |
| | | GTTCCATGGA TGAACTGAAT CATGATTTTC AAGCACTTGC | |
| | | TCTGGAGGGA AGAGCGATGG GAGAGCAGCT CTTGCCAGGT | |
| | | AAAAAGTTTT GGGAAACAGA TGAATCCAGC AAAGATGGAC | |
| | | CAAAAGGAAT ATTCCTGGGT GATCAATGGC GAGACAGTGC | |
| | | CTGGGGAACA TCAGATCATT CAGTTTCCCA GCCAATCATG | |
| | | GTGCAGAGAA GACCTGGTCA GAGTTTCCAT GTGAACAGTG | |
| | | AGGTCAATTC TGTACTGTCC CCACGATCGG AGAGTGGGGG | |
| | | ACTAGGCGTT AGCATGGTGG AGTATGTGTT GAGCTCATCC | |
| | | CCGGGCGATT CCTGTCTAAG AAAAGGAGGA TTTGGCCCAA | |
| | | GGGATGCAGA CAGTGATGAA AACGACAAAG GTGAAAAGAA | |
| | | GAACAAGGGT ACGTTTGATG GAGATAAGCT AGGAGATTTG | |
| | | AAGGAGGAGG GTGATGTGAT GGACAAGACC AATGGTTTAC | |
| | | CAGTGCAGAA TGGGATTGAT GCAGACGTCA AAGATTTTAG | |
| | | CCGTACCCCT GGTAATTGCC AGAACTCTGC TAATGAAGTG | |
| | | GATCTTCTGG GTCCAAACCA GAATGGTTCT GAGGGCTTAG | |
| | | CCCAGCTGAC CAGCACCAAT GGTGCCAAGC CTGTGGAGGA | |
| | | TTTCTCCAAC ATGGAGTCCC AGAGTGTCCC CTTGGACCCC | |
| | | ATGGAACATG TGGGCATGGA GCCTCTTCAG TTTGATTATT | |
| | | CAGGCACGCA GGTACCTGTG GACTCAGCAG CAGCAACTGT | |
| | | GGGACTTTTT GACTACAATT CTCAACAACA GCTGTTCCAA | |
| | | AGACCTAATG CGCTTGCTGT CCAGCAGTTG ACAGCTGCTC | |
| | | AGCAGCAGCA GTATGCACTG GCAGCTGCTC ATCAGCCGCA | |
| | | CATCGGTTTA GCTCCCGCTG CGTTTGTCCC CAATCCATAC | |
| | | ATCATCAGCG CTGCTCCCCC AGGGACGGAC CCCTACACAG | |
| | | CTGGATTGGC TGCAGCAGCG ACACTAGGCC CAGCTGTGGT | |
| | | CCCTCACCAG TATTATGGAG TTACTCCCTG GGGAGTCTAC | |
| | | CCTGCCAGTC TTTTCCAGCA GCAAGCTGCC GCTGCCGCTG | |
| | | CAGCAACTAA TTCAGCTAAT CAACAGACCA CCCCACAGGC | |
| | | TCAGCAAGGA CAGCAGCAGG TTCTCCGTGG AGGAGCCAGC | |
| | | CAACGTCCTT TGACCCCAAA CCAGAACCAG CAGGGACAGC | |
| | | AAACGGATCC CCTTGTGGCA GCTGCAGCAG TGAATTCTGC | |
| | | CCTTGCATTT GGACAAGGTC TGGCAGCAGG CATGCCAGGT | |
| | | TATCCGGTGT TGGCTCCTGC TGCTTACTAT GACCAAACTG | |
| | | GTGCCCTTGT AGTGAATGCA GGCGCGAGAA ATGGTCTTGG | |
| | | AGCTCCTGTT CGACTTGTAG CTCCTGCCCC AGTCATCATT | |
| | | AGTTCCTCAG CTGCACAAGC AGCTGTTGCA GCAGCCGCAG | |
| | | CTTCAGCAAA TGGAGCAGCT GGTGGTCTTG CTGGAACAAC | |
| | | AAATGGACCA TTTCGCCCTT TAGGAACACA GCAGCCTCAG | |
| | | CCCCAGCCCC AGCAGCAGCC CAATAACAAC CTGGCATCCA | |
| | | GTTCTTTCTA CGGCAACAAC TCTCTGAACA GCAATTCACA | |
| | | GAGCAGCTCC CTCTTCTCCC AGGGCTCTGC CCAGCCTGCC | |
| | | AACACATCCT TGGGATTCGG AAGTAGCAGT TCTCTCGGCG | |
| | | CCACCCTGGG ATCCGCCCTT GGAGGGTTTG GAACAGCAGT | |
| | | TGCAAACTCC AACACTGGCA GTGGCTCCCG CCGTGACTCC | |
| | | CTGACTGGCA GCAGTGACCT TTATAAGAGG ACATCGAGCA | |
| | | GCTTGACCCC CATTGGACAC AGTTTTTATA ACGGCCTTAG | |
| | | CTTTTCCTCC TCTCCTGGAC CCGTGGGCAT GCCTCTCCCT | |
| | | AGTCAGGGAC CAGGACATTC ACAGACACCA CCTCCTTCCC | |
| | | TCTCTTCACA TGGATCCTCT TCAAGCTTAA ACCTGGGAGG | |
| | | ACTCACGAAT GGCAGTGGAA GATACATCTC TGCTGCTCCA | |
| | | GGCGCTGAAG CCAAGTACCG CAGTGCAAGC AGCGCCTCCA | |
| | | GCCTCTTCAG CCCGAGCAGC ACTCTTTTCT CTTCCTCTCG | |
| | | TTTGCGATAT GGAATGTCTG ATGTCATGCC TTCTGGCAGG | |
| | | AGCAGGCTTT TGGAAGATTT TCGAAACAAC CGGTACCCCA | |
| | | ATTTACAACT GCGGGAGATT GCTGGACATA TAATGGAATT | |
| | | TTCCCAAGAC CAGCATGGGT CCAGATTCAT TCAGCTGAAA | |
| | | CTGGAGCGTG CCACACCAGC TGAGCGCCAG CTTGTCTTCA | |
| | | ATGAAATCCT CCAGGCTGCC TACCAACTCA TGGTGGATGT | |
| | | GTTTGGTAAT TACGTCATTC AGAAGTTCTT TGAATTTGGC | |
| | | AGTCTTGAAC AGAAGCTGGC TTTGGCAGAA CGGATTCGAG | |
| | | GCCACGTCCT GTCATTGGCA CTACAGATGT ATGGCTGCCG | |
| | | TGTTATCCAG AAAGCTCTTG AGTTTATTCC TTCAGACCAG | |
| | | CAGGTAATTA ATGAGATGGT TCGGGAACTA GATGGCCATG | |
| | | TCTTGAAGTG TGTGAAAGAT CAGAATGGCA ATCACGTGGT | |
| | | TCAGAAATGC ATTGAATGTG TACAGCCCCA GTCTTTGCAA | |
| | | TTTATCATCG ATGCGTTTAA GGGACAGGTA TTTGCCTTAT | |
| | | CCACACATCC TTATGGCTGC CGAGTGATTC AGAGAATCCT | |
| | | GGAGCACTGT CTCCCTGACC AGACACTCCC TATTTTAGAG | |
| | | GAGCTTCACC AGCACACAGA GCAGCTTGTA CAGGATCAAT | |
| | | ATGGAAATTA TGTAATCCAA CATGTACTGG AGCACGGTCG | |
| | | TCCTGAGGAT AAAAGCAAAA TTGTAGCAGA AATCCGAGGC | |
| | | AATGTACTTG TATTGAGTCA GCACAAATTT GCAAGCAATG | |
| | | TTGTGGAGAA GTGTGTTACT CACGCCTCAC GTACGGAGCG | |
| | | CGCTGTGCTC ATCGATGAGG TGTGCACCAT GAACGACGGT | |
| | | CCCCACAGTG CCTTATACAC CATGATGAAG GACCAGTATG | |
| | | CCAACTACGT GGTCCAGAAG ATGATTGACG TGGCGGAGCC | |
| | | AGGCCAGCGG AAGATCGTCA TGCATAAGAT CCGGCCCCAC | |
| | | ATCGCAACTC TTCGTAAGTA CACCTATGGC AAGCACATTC | |
| | | TGGCCAAGCT GGAGAAGTAC TACATGAAGA ACGGTGTTGA | |
| | | CTTAGGGCCC ATCTGTGGCC CCCCTAATGG TATCATCTGA | |
| | | GGCAGTGTCA CCCGCTGTTC CCTCATTCCC GCTGACCTCA | |
| | | CTGGCCCACT GGCAAATCCA ACCAGCAACC AGAAATGTTC | |
| | | TAGTGTAGAG TCTGAGACGG GCAAGTGGTT GCTCCAGGAT | |
| | | TACTCCCTCC TCCAAAAAAG GAATCAAATC CACGAGTGGA | |
| | | AAAGCCTTTG TAAATTTAAT TTTATTACAC ATAACATGTA | |
| | | CTATTTTTTT TAATTGACTA ATTGCCCTGC TGTTTTACTG | |
| | | GTGTATAGGA TACTTGTACA TAGGTAACCA ATGTACATGG | |
| | | GAGGCCACAT ATTTTGTTCA CTGTTGTATC TATATTTCAC | |
| | | ATGTGGAAAC TTTCAGGGTG GTTGGTTTAA CAAAAAAAAA | |
| | | AAGCTTTAAA AAAAAAAGAA AAAAAGGAAA AGGTTTTTAG | |
| | | CTCATTTGCC TGGCCGGCAA GTTTTGCAAA TAGCTCTTCC | |
| | | CCACCTCCTC ATTTTAGTAA AAAACAAACA AAAACAAAAA | |
| | | AACCTGAGAA GTTTGAATTG TAGTTAAATG ACCCCAAACT | |
| | | GGCATTTAAC ACTGTTTATA AAAAATATAT ATATATATAT | |
| | | ATATATATAT AATGAAAAAG GTTTCAGAGT TGCTAAAGCT | |
| | | TCAGTTTGTG ACATTAAGTT TATGAAATTC TAAAAAATGC | |
| | | CTTTTTTGGA GACTATATTA TGCTGAAGAA GGCTGTTCGT | |
| | | GAGGAGGAGA TGCGAGCACC CAGAACGTCT TTTGAGGCTG | |
| | | GGCGGGTGTG ATTGTTTACT GCCTACTGGA TTTTTTTCTA | |
| | | TTAACATTGA AAGGTAAAAT CTGATTATTT AGCATGAGAA | |
| | | AAAAAAATCC AACTCTGCTT TTGGTCTTGC TTCTATAAAT | |
| | | ATATAGTGTA TACTTGGTGT AGACTTTGCA TATATACAAA | |
| | | TTTGTAGTAT TTTCTTGTTT TGATGTCTAA TCTGTATCTA | |
| | | TAATGTACCC TAGTAGTCGA ACATACTTTT GATTGTACAA | |
| | | TTGTACATTT GTATACCTGT AATGTAAATG TGGAGAAGTT | |
| | | TGAATCAACA TAAACACGTT TTTTGGTAAG AAAAGAGAAT | |
| | | TAGCCAGCCC TGTGCATTCA GTGTATATTC TCACCTTTTA | |
| | | TGGTCGTAGC ATATAGTGTT GTATATTGTA AATTGTAATT | |
| | | TCAACCAGAA GTAAATTTTT TTCTTTTGAA GGAATAAATG | |
| | | TTCTTTATAC AGCCTAGTTA ATGTTTAAAA AGAAAAAAAT | |
| | | AGCTTGGTTT TATTTGTCAT CTAGTCTCAA GTATAGCGAG | |
| | | ATTCTTTCTA AATGTTATTC AAGATTGAGT TCTCACTAGT | |
| | | GTTTTTTTAA TCCTAAAAAA GTAATGTTTT GATTTTGTGA | |
| | | CAGTCAAAAG GACGTGCAAA AGTCTAGCCT TGCCCGAGCT | |
| | | TTCCTTACAA TCAGAGCCCC TCTCACCTTG TAAAGTGTGA | |
| | | ATCGCCCTTC CCTTTTGTAC AGAAGATGAA CTGTATTTTG | |
| | | CATTTTGTCT ACTTGTAAGT GAATGTAACA TACTGTCAAT | |
| | | TTTCCTTGTT TGAATATAGA ATTGTAACAC TACACGGTGT | |
| | | ACATTTCCAG AGCCTTGTGT ATATTTCCAA TGAACTTTTT | |
| | | TGCAAGCACA CTTGTAACCA TATGTGTATA ATTAACAAAC | |
| | | CTGTGTATGC TTATGCCTGG GCAACTATTT TTTGTAACTC | |
| | | TTGTGTAGAT TGTCTCTAAA CAATGTGTGA TCTTTATTTT | |
| | | GAAAAATACA GAACTTTGGA ATCTGAAAAA AAAAAAAAAA | |
| | | AAAAAAAAAA AAAAAA | |
| ACTB | NM_001101.4 | GAGTGAGCGG CGCGGGGCCA ATCAGCGTGC GCCGTTCCGA | 41 |
| | | AAGTTGCCTT TTATGGCTCG AGCGGCCGCG GCGGCGCCCT | |
| | | ATAAAACCCA GCGGCGCGAC GCGCCACCAC CGCCGAGACC | |
| | | GCGTCCGCCC CGCGAGCACA GAGCCTCGCC TTTGCCGATC | |
| | | CGCCGCCCGT CCACACCCGC CGCCAGCTCA CCATGGATGA | |
| | | TGATATCGCC GCGCTCGTCG TCGACAACGG CTCCGGCATG | |
| | | TGCAAGGCCG GCTTCGCGGG CGACGATGCC CCCCGGGCCG | |
| | | TCTTCCCCTC CATCGTGGGG CGCCCCAGGC ACCAGGGCGT | |
| | | GATGGTGGGC ATGGGTCAGA AGGATTCCTA TGTGGGCGAC | |
| | | GAGGCCCAGA GCAAGAGAGG CATCCTCACC CTGAAGTACC | |
| | | CCATCGAGCA CGGCATCGTC ACCAACTGGG ACGACATGGA | |
| | | GAAAATCTGG CACCACACCT TCTACAATGA GCTGCGTGTG | |
| | | GCTCCCGAGG AGCACCCCGT GCTGCTGACC GAGGCCCCCC | |
| | | TGAACCCCAA GGCCAACCGC GAGAAGATGA CCCAGATCAT | |
| | | GTTTGAGACC TTCAACACCC CAGCCATGTA CGTTGCTATC | |
| | | CAGGCTGTGC TATCCCTGTA CGCCTCTGGC CGTACCACTG | |
| | | GCATCGTGAT GGACTCCGGT GACGGGGTCA CCCACACTGT | |
| | | GCCCATCTAC GAGGGGTATG CCCTCCCCCA TGCCATCCTG | |
| | | CGTCTGGACC TGGCTGGCCG GGACCTGACT GACTACCTCA | |
| | | TGAAGATCCT CACCGAGCGC GGCTACAGCT TCACCACCAC | |
| | | GGCCGAGCGG GAAATCGTGC GTGACATTAA GGAGAAGCTG | |
| | | TGCTACGTCG CCCTGGACTT CGAGCAAGAG ATGGCCACGG | |
| | | CTGCTTCCAG CTCCTCCCTG GAGAAGAGCT ACGAGCTGCC | |
| | | TGACGGCCAG GTCATCACCA TTGGCAATGA GCGGTTCCGC | |
| | | TGCCCTGAGG CACTCTTCCA GCCTTCCTTC CTGGGCATGG | |
| | | AGTCCTGTGG CATCCACGAA ACTACCTTCA ACTCCATCAT | |
| | | GAAGTGTGAC GTGGACATCC GCAAAGACCT GTACGCCAAC | |
| | | ACAGTGCTGT CTGGCGGCAC CACCATGTAC CCTGGCATTG | |
| | | CCGACAGGAT GCAGAAGGAG ATCACTGCCC TGGCACCCAG | |
| | | CACAATGAAG ATCAAGATCA TTGCTCCTCC TGAGCGCAAG | |
| | | TACTCCGTGT GGATCGGCGG CTCCATCCTG GCCTCGCTGT | |
| | | CCACCTTCCA GCAGATGTGG ATCAGCAAGC AGGAGTATGA | |
| | | CGAGTCCGGC CCCTCCATCG TCCACCGCAA ATGCTTCTAG | |
| | | GCGGACTATG ACTTAGTTGC GTTACACCCT TTCTTGACAA | |
| | | AACCTAACTT GCGCAGAAAA CAAGATGAGA TTGGCATGGC | |
| | | TTTATTTGTT TTTTTTGTTT TGTTTTGGTT TTTTTTTTTT | |
| | | TTTTGGCTTG ACTCAGGATT TAAAAACTGG AACGGTGAAG | |
| | | GTGACAGCAG TCGGTTGGAG CGAGCATCCC CCAAAGTTCA | |
| | | CAATGTGGCC GAGGACTTTG ATTGCACATT GTTGTTTTTT | |
| | | TAATAGTCAT TCCAAATATG AGATGCGTTG TTACAGGAAG | |
| | | TCCCTTGCCA TCCTAAAAGC CACCCCACTT CTCTCTAAGG | |
| | | AGAATGGCCC AGTCCTCTCC CAAGTCCACA CAGGGGAGGT | |
| | | GATAGCATTG CTTTCGTGTA AATTATGTAA TGCAAAATTT | |
| | | TTTTAATCTT CGCCTTAATA CTTTTTTATT TTGTTTTATT | |
| | | TTGAATGATG AGCCTTCGTG CCCCCCCTTC CCCCTTTTTT | |
| | | GTCCCCCAAC TTGAGATGTA TGAAGGCTTT TGGTCTCCCT | |
| | | GGGAGTGGGT GGAGGCAGCC AGGGCTTACC TGTACACTGA | |
| | | CTTGAGACCA GTTGAATAAA AGTGCACACC TTAAAAATGA | |
| | | GGAAAAAAAA AAAAAAAAAA | |
| GAPD | NM_002046.6 | GCTCTCTGCT CCTCCTGTTC GACAGTCAGC CGCATCTTCT | 42 |
| | | TTTGCGTCGC CAGCCGAGCC ACATCGCTCA GACACCATGG | |
| | | GGAAGGTGAA GGTCGGAGTC AACGGATTTG GTCGTATTGG | |
| | | GCGCCTGGTC ACCAGGGCTG CTTTTAACTC TGGTAAAGTG | |
| | | GATATTGTTG CCATCAATGA CCCCTTCATT GACCTCAACT | |
| | | ACATGGTTTA CATGTTCCAA TATGATTCCA CCCATGGCAA | |
| | | ATTCCATGGC ACCGTCAAGG CTGAGAACGG GAAGCTTGTC | |
| | | ATCAATGGAA ATCCCATCAC CATCTTCCAG GAGCGAGATC | |
| | | CCTCCAAAAT CAAGTGGGGC GATGCTGGCG CTGAGTACGT | |
| | | CGTGGAGTCC ACTGGCGTCT TCACCACCAT GGAGAAGGCT | |
| | | GGGGCTCATT TGCAGGGGGG AGCCAAAAGG GTCATCATCT | |
| | | CTGCCCCCTC TGCTGATGCC CCCATGTTCG TCATGGGTGT | |
| | | GAACCATGAG AAGTATGACA ACAGCCTCAA GATCATCAGC | |
| | | AATGCCTCCT GCACCACCAA CTGCTTAGCA CCCCTGGCCA | |
| | | AGGTCATCCA TGACAACTTT GGTATCGTGG AAGGACTCAT | |
| | | GACCACAGTC CATGCCATCA CTGCCACCCA GAAGACTGTG | |
| | | GATGGCCCCT CCGGGAAACT GTGGCGTGAT GGCCGCGGGG | |
| | | CTCTCCAGAA CATCATCCCT GCCTCTACTG GCGCTGCCAA | |
| | | GGCTGTGGGC AAGGTCATCC CTGAGCTGAA CGGGAAGCTC | |
| | | ACTGGCATGG CCTTCCGTGT CCCCACTGCC AACGTGTCAG | |
| | | TGGTGGACCT GACCTGCCGT CTAGAAAAAC CTGCCAAATA | |
| | | TGATGACATC AAGAAGGTGG TGAAGCAGGC GTCGGAGGGC | |
| | | CCCCTCAAGG GCATCCTGGG CTACACTGAG CACCAGGTGG | |
| | | TCTCCTCTGA CTTCAACAGC GACACCCACT CCTCCACCTT | |
| | | TGACGCTGGG GCTGGCATTG CCCTCAACGA CCACTTTGTC | |
| | | AAGCTCATTT CCTGGTATGA CAACGAATTT GGCTACAGCA | |
| | | ACAGGGTGGT GGACCTCATG GCCCACATGG CCTCCAAGGA | |
| | | GTAAGACCCC TGGACCACCA GCCCCAGCAA GAGCACAAGA | |
| | | GGAAGAGAGA GACCCTCACT GCTGGGGAGT CCCTGCCACA | |
| | | CTCAGTCCCC CACCACACTG AATCTCCCCT CCTCACAGTT | |
| | | GCCATGTAGA CCCCTTGAAG AGGGGAGGGG CCTAGGGAGC | |
| | | CGCACCTTGT CATGTACCAT CAATAAAGTA CCCTGTGCTC | |
| | | AACCAGTTAA AAAAAAAAAA AAAAAAAAA | |
| GUSB | NM_000181.3 | GTCCTCAACC AAGATGGCGC GGATGGCTTC AGGCGCATCA | 43 |
| | | CGACACCGGC GCGTCACGCG ACCCGCCCTA CGGGCACCTC | |
| | | CCGCGCTTTT CTTAGCGCCG CAGACGGTGG CCGAGCGGGG | |
| | | GACCGGGAAG CATGGCCCGG GGGTCGGCGG TTGCCTGGGC | |
| | | GGCGCTCGGG CCGTTGTTGT GGGGCTGCGC GCTGGGGCTG | |
| | | CAGGGCGGGA TGCTGTACCC CCAGGAGAGC CCGTCGCGGG | |
| | | AGTGCAAGGA GCTGGACGGC CTCTGGAGCT TCCGCGCCGA | |
| | | CTTCTCTGAC AACCGACGCC GGGGCTTCGA GGAGCAGTGG | |
| | | TACCGGCGGC CGCTGTGGGA GTCAGGCCCC ACCGTGGACA | |
| | | TGCCAGTTCC CTCCAGCTTC AATGACATCA GCCAGGACTG | |
| | | GCGTCTGCGG CATTTTGTCG GCTGGGTGTG GTACGAACGG | |
| | | GAGGTGATCC TGCCGGAGCG ATGGACCCAG GACCTGCGCA | |
| | | CAAGAGTGGT GCTGAGGATT GGCAGTGCCC ATTCCTATGC | |
| | | CATCGTGTGG GTGAATGGGG TCGACACGCT AGAGCATGAG | |
| | | GGGGGCTACC TCCCCTTCGA GGCCGACATC AGCAACCTGG | |
| | | TCCAGGTGGG GCCCCTGCCC TCCCGGCTCC GAATCACTAT | |
| | | CGCCATCAAC AACACACTCA CCCCCACCAC CCTGCCACCA | |
| | | GGGACCATCC AATACCTGAC TGACACCTCC AAGTATCCCA | |
| | | AGGGTTACTT TGTCCAGAAC ACATATTTTG ACTTTTTCAA | |
| | | CTACGCTGGA CTGCAGCGGT CTGTACTTCT GTACACGACA | |
| | | CCCACCACCT ACATCGATGA CATCACCGTC ACCACCAGCG | |
| | | TGGAGCAAGA CAGTGGGCTG GTGAATTACC AGATCTCTGT | |
| | | CAAGGGCAGT AACCTGTTCA AGTTGGAAGT GCGTCTTTTG | |
| | | GATGCAGAAA ACAAAGTCGT GGCGAATGGG ACTGGGACCC | |
| | | AGGGCCAACT TAAGGTGCCA GGTGTCAGCC TCTGGTGGCC | |
| | | GTACCTGATG CACGAACGCC CTGCCTATCT GTATTCATTG | |
| | | GAGGTGCAGC TGACTGCACA GACGTCACTG GGGCCTGTGT | |
| | | CTGACTTCTA CACACTCCCT GTGGGGATCC GCACTGTGGC | |
| | | TGTCACCAAG AGCCAGTTCC TCATCAATGG GAAACCTTTC | |
| | | TATTTCCACG GTGTCAACAA GCATGAGGAT GCGGACATCC | |
| | | GAGGGAAGGG CTTCGACTGG CCGCTGCTGG TGAAGGACTT | |
| | | CAACCTGCTT CGCTGGCTTG GTGCCAACGC TTTCCGTACC | |
| | | AGCCACTACC CCTATGCAGA GGAAGTGATG CAGATGTGTG | |
| | | ACCGCTATGG GATTGTGGTC ATCGATGAGT GTCCCGGCGT | |
| | | GGGCCTGGCG CTGCCGCAGT TCTTCAACAA CGTTTCTCTG | |
| | | CATCACCACA TGCAGGTGAT GGAAGAAGTG GTGCGTAGGG | |
| | | ACAAGAACCA CCCCGCGGTC GTGATGTGGT CTGTGGCCAA | |
| | | CGAGCCTGCG TCCCACCTAG AATCTGCTGG CTACTACTTG | |
| | | AAGATGGTGA TCGCTCACAC CAAATCCTTG GACCCCTCCC | |
| | | GGCCTGTGAC CTTTGTGAGC AACTCTAACT ATGCAGCAGA | |
| | | CAAGGGGGCT CCGTATGTGG ATGTGATCTG TTTGAACAGC | |
| | | TACTACTCTT GGTATCACGA CTACGGGCAC CTGGAGTTGA | |
| | | TTCAGCTGCA GCTGGCCACC CAGTTTGAGA ACTGGTATAA | |
| | | GAAGTATCAG AAGCCCATTA TTCAGAGCGA GTATGGAGCA | |
| | | GAAACGATTG CAGGGTTTCA CCAGGATCCA CCTCTGATGT | |
| | | TCACTGAAGA GTACCAGAAA AGTCTGCTAG AGCAGTACCA | |
| | | TCTGGGTCTG GATCAAAAAC GCAGAAAATA CGTGGTTGGA | |
| | | GAGCTCATTT GGAATTTTGC CGATTTCATG ACTGAACAGT | |
| | | CACCGACGAG AGTGCTGGGG AATAAAAAGG GGATCTTCAC | |
| | | TCGGCAGAGA CAACCAAAAA GTGCAGCGTT CCTTTTGCGA | |
| | | GAGAGATACT GGAAGATTGC CAATGAAACC AGGTATCCCC | |
| | | ACTCAGTAGC CAAGTCACAA TGTTTGGAAA ACAGCCTGTT | |
| | | TACTTGAGCA AGACTGATAC CACCTGCGTG TCCCTTCCTC | |
| | | CCCGAGTCAG GGCGACTTCC ACAGCAGCAG AACAAGTGCC | |
| | | TCCTGGACTG TTCACGGCAG ACCAGAACGT TTCTGGCCTG | |
| | | GGTTTTGTGG TCATCTATTC TAGCAGGGAA CACTAAAGGT | |
| | | GGAAATAAAA GATTTTCTAT TATGGAAATA AAGAGTTGGC | |
| | | ATGAAAGTGG CTACTGAAAA AAAAAAAAAA AAAAAAAAAA A | |
| RPLP0 | NM_001002.3 | GTCTGACGGG CGATGGCGCA GCCAATAGAC AGGAGCGCTA | 44 |
| | | TCCGCGGTTT CTGATTGGCT ACTTTGTTCG CATTATAAAA | |
| | | GGCACGCGCG GGCGCGAGGC CCTTCTCTCG CCAGGCGTCC | |
| | | TCGTGGAAGT GACATCGTCT TTAAACCCTG CGTGGCAATC | |
| | | CCTGACGCAC CGCCGTGATG CCCAGGGAAG ACAGGGCGAC | |
| | | CTGGAAGTCC AACTACTTCC TTAAGATCAT CCAACTATTG | |
| | | GATGATTATC CGAAATGTTT CATTGTGGGA GCAGACAATG | |
| | | TGGGCTCCAA GCAGATGCAG CAGATCCGCA TGTCCCTTCG | |
| | | CGGGAAGGCT GTGGTGCTGA TGGGCAAGAA CACCATGATG | |
| | | CGCAAGGCCA TCCGAGGGCA CCTGGAAAAC AACCCAGCTC | |
| | | TGGAGAAACT GCTGCCTCAT ATCCGGGGGA ATGTGGGCTT | |
| | | TGTGTTCACC AAGGAGGACC TCACTGAGAT CAGGGACATG | |
| | | TTGCTGGCCA ATAAGGTGCC AGCTGCTGCC CGTGCTGGTG | |
| | | CCATTGCCCC ATGTGAAGTC ACTGTGCCAG CCCAGAACAC | |
| | | TGGTCTCGGG CCCGAGAAGA CCTCCTTTTT CCAGGCTTTA | |
| | | GGTATCACCA CTAAAATCTC CAGGGGCACC ATTGAAATCC | |
| | | TGAGTGATGT GCAGCTGATC AAGACTGGAG ACAAAGTGGG | |
| | | AGCCAGCGAA GCCACGCTGC TGAACATGCT CAACATCTCC | |
| | | CCCTTCTCCT TTGGGCTGGT CATCCAGCAG GTGTTCGACA | |
| | | ATGGCAGCAT CTACAACCCT GAAGTGCTTG ATATCACAGA | |
| | | GGAAACTCTG CATTCTCGCT TCCTGGAGGG TGTCCGCAAT | |
| | | GTTGCCAGTG TCTGTCTGCA GATTGGCTAC CCAACTGTTG | |
| | | CATCAGTACC CCATTCTATC ATCAACGGGT ACAAACGAGT | |
| | | CCTGGCCTTG TCTGTGGAGA CGGATTACAC CTTCCCACTT | |
| | | GCTGAAAAGG TCAAGGCCTT CTTGGCTGAT CCATCTGCCT | |
| | | TTGTGGCTGC TGCCCCTGTG GCTGCTGCCA CCACAGCTGC | |
| | | TCCTGCTGCT GCTGCAGCCC CAGCTAAGGT TGAAGCCAAG | |
| | | GAAGAGTCGG AGGAGTCGGA CGAGGATATG GGATTTGGTC | |
| | | TCTTTGACTA ATCACCAAAA AGCAACCAAC TTAGCCAGTT | |
| | | TTATTTGCAA AACAAGGAAA TAAAGGCTTA CTTCTTTAAA | |
| | | AAGTAAAAAA AAAAAAAAAA AAAAAAAAA | |
| TFRC | NM_003234.3 | AGAGCGTCGG GATATCGGGT GGCGGCTCGG GACGGAGGAC | 45 |
| | | GCGCTAGTGT GAGTGCGGGC TTCTAGAACT ACACCGACCC | |
| | | TCGTGTCCTC CCTTCATCCT GCGGGGCTGG CTGGAGCGGC | |
| | | CGCTCCGGTG CTGTCCAGCA GCCATAGGGA GCCGCACGGG | |
| | | GAGCGGGAAA GCGGTCGCGG CCCCAGGCGG GGCGGCCGGG | |
| | | ATGGAGCGGG GCCGCGAGCC TGTGGGGAAG GGGCTGTGGC | |
| | | GGCGCCTCGA GCGGCTGCAG GTTCTTCTGT GTGGCAGTTC | |
| | | AGAATGATGG ATCAAGCTAG ATCAGCATTC TCTAACTTGT | |
| | | TTGGTGGAGA ACCATTGTCA TATACCCGGT TCAGCCTGGC | |
| | | TCGGCAAGTA GATGGCGATA ACAGTCATGT GGAGATGAAA | |
| | | CTTGCTGTAG ATGAAGAAGA AAATGCTGAC AATAACACAA | |
| | | AGGCCAATGT CACAAAACCA AAAAGGTGTA GTGGAAGTAT | |
| | | CTGCTATGGG ACTATTGCTG TGATCGTCTT TTTCTTGATT | |
| | | GGATTTATGA TTGGCTACTT GGGCTATTGT AAAGGGGTAG | |
| | | AACCAAAAAC TGAGTGTGAG AGACTGGCAG GAACCGAGTC | |
| | | TCCAGTGAGG GAGGAGCCAG GAGAGGACTT CCCTGCAGCA | |
| | | CGTCGCTTAT ATTGGGATGA CCTGAAGAGA AAGTTGTCGG | |
| | | AGAAACTGGA CAGCACAGAC TTCACCGGCA CCATCAAGCT | |
| | | GCTGAATGAA AATTCATATG TCCCTCGTGA GGCTGGATCT | |
| | | CAAAAAGATG AAAATCTTGC GTTGTATGTT GAAAATCAAT | |
| | | TTCGTGAATT TAAACTCAGC AAAGTCTGGC GTGATCAACA | |
| | | TTTTGTTAAG ATTCAGGTCA AAGACAGCGC TCAAAACTCG | |
| | | GTGATCATAG TTGATAAGAA CGGTAGACTT GTTTACCTGG | |
| | | TGGAGAATCC TGGGGGTTAT GTGGCGTATA GTAAGGCTGC | |
| | | AACAGTTACT GGTAAACTGG TCCATGCTAA TTTTGGTACT | |
| | | AAAAAAGATT TTGAGGATTT ATACACTCCT GTGAATGGAT | |
| | | CTATAGTGAT TGTCAGAGCA GGGAAAATCA CCTTTGCAGA | |
| | | AAAGGTTGCA AATGCTGAAA GCTTAAATGC AATTGGTGTG | |
| | | TTGATATACA TGGACCAGAC TAAATTTCCC ATTGTTAACG | |
| | | CAGAACTTTC ATTCTTTGGA CATGCTCATC TGGGGACAGG | |
| | | TGACCCTTAC ACACCTGGAT TCCCTTCCTT CAATCACACT | |
| | | CAGTTTCCAC CATCTCGGTC ATCAGGATTG CCTAATATAC | |
| | | CTGTCCAGAC AATCTCCAGA GCTGCTGCAG AAAAGCTGTT | |
| | | TGGGAATATG GAAGGAGACT GTCCCTCTGA CTGGAAAACA | |
| | | GACTCTACAT GTAGGATGGT AACCTCAGAA AGCAAGAATG | |
| | | TGAAGCTCAC TGTGAGCAAT GTGCTGAAAG AGATAAAAAT | |
| | | TCTTAACATC TTTGGAGTTA TTAAAGGCTT TGTAGAACCA | |
| | | GATCACTATG TTGTAGTTGG GGCCCAGAGA GATGCATGGG | |
| | | GCCCTGGAGC TGCAAAATCC GGTGTAGGCA CAGCTCTCCT | |
| | | ATTGAAACTT GCCCAGATGT TCTCAGATAT GGTCTTAAAA | |
| | | GATGGGTTTC AGCCCAGCAG AAGCATTATC TTTGCCAGTT | |
| | | GGAGTGCTGG AGACTTTGGA TCGGTTGGTG CCACTGAATG | |
| | | GCTAGAGGGA TACCTTTCGT CCCTGCATTT AAAGGCTTTC | |
| | | ACTTATATTA ATCTGGATAA AGCGGTTCTT GGTACCAGCA | |
| | | ACTTCAAGGT TTCTGCCAGC CCACTGTTGT ATACGCTTAT | |
| | | TGAGAAAACA ATGCAAAATG TGAAGCATCC GGTTACTGGG | |
| | | CAATTTCTAT ATCAGGACAG CAACTGGGCC AGCAAAGTTG | |
| | | AGAAACTCAC TTTAGACAAT GCTGCTTTCC CTTTCCTTGC | |
| | | ATATTCTGGA ATCCCAGCAG TTTCTTTCTG TTTTTGCGAG | |
| | | GACACAGATT ATCCTTATTT GGGTACCACC ATGGACACCT | |
| | | ATAAGGAACT GATTGAGAGG ATTCCTGAGT TGAACAAAGT | |
| | | GGCACGAGCA GCTGCAGAGG TCGCTGGTCA GTTCGTGATT | |
| | | AAACTAACCC ATGATGTTGA ATTGAACCTG GACTATGAGA | |
| | | GGTACAACAG CCAACTGCTT TCATTTGTGA GGGATCTGAA | |
| | | CCAATACAGA GCAGACATAA AGGAAATGGG CCTGAGTTTA | |
| | | CAGTGGCTGT ATTCTGCTCG TGGAGACTTC TTCCGTGCTA | |
| | | CTTCCAGACT AACAACAGAT TTCGGGAATG CTGAGAAAAC | |
| | | AGACAGATTT GTCATGAAGA AACTCAATGA TCGTGTCATG | |
| | | AGAGTGGAGT ATCACTTCCT CTCTCCCTAC GTATCTCCAA | |
| | | AAGAGTCTCC TTTCCGACAT GTCTTCTGGG GCTCCGGCTC | |
| | | TCACACGCTG CCAGCTTTAC TGGAGAACTT GAAACTGCGT | |
| | | AAACAAAATA ACGGTGCTTT TAATGAAACG CTGTTCAGAA | |
| | | ACCAGTTGGC TCTAGCTACT TGGACTATTC AGGGAGCTGC | |
| | | AAATGCCCTC TCTGGTGACG TTTGGGACAT TGACAATGAG | |
| | | TTTTAAATGT GATACCCATA GCTTCCATGA GAACAGCAGG | |
| | | GTAGTCTGGT TTCTAGACTT GTGCTGATCG TGCTAAATTT | |
| | | TCAGTAGGGC TACAAAACCT GATGTTAAAA TTCCATCCCA | |
| | | TCATCTTGGT ACTACTAGAT GTCTTTAGGC AGCAGCTTTT | |
| | | AATACAGGGT AGATAACCTG TACTTCAAGT TAAAGTGAAT | |
| | | AACCACTTAA AAAATGTCCA TGATGGAATA TTCCCCTATC | |
| | | TCTAGAATTT TAAGTGCTTT GTAATGGGAA CTGCCTCTTT | |
| | | CCTGTTGTTG TTAATGAAAA TGTCAGAAAC CAGTTATGTG | |
| | | AATGATCTCT CTGAATCCTA AGGGCTGGTC TCTGCTGAAG | |
| | | GTTGTAAGTG GTCGCTTACT TTGAGTGATC CTCCAACTTC | |
| | | ATTTGATGCT AAATAGGAGA TACCAGGTTG AAAGACCTTC | |
| | | TCCAAATGAG ATCTAAGCCT TTCCATAAGG AATGTAGCTG | |
| | | GTTTCCTCAT TCCTGAAAGA AACAGTTAAC TTTCAGAAGA | |
| | | GATGGGCTTG TTTTCTTGCC AATGAGGTCT GAAATGGAGG | |
| | | TCCTTCTGCT GGATAAAATG AGGTTCAACT GTTGATTGCA | |
| | | GGAATAAGGC CTTAATATGT TAACCTCAGT GTCATTTATG | |
| | | AAAAGAGGGG ACCAGAAGCC AAAGACTTAG TATATTTTCT | |
| | | TTTCCTCTGT CCCTTCCCCC ATAAGCCTCC ATTTAGTTCT | |
| | | TTGTTATTTT TGTTTCTTCC AAAGCACATT GAAAGAGAAC | |
| | | CAGTTTCAGG TGTTTAGTTG CAGACTCAGT TTGTCAGACT | |
| | | TTAAAGAATA ATATGCTGCC AAATTTTGGC CAAAGTGTTA | |
| | | ATCTTAGGGG AGAGCTTTCT GTCCTTTTGG CACTGAGATA | |
| | | TTTATTGTTT ATTTATCAGT GACAGAGTTC ACTATAAATG | |
| | | GTGTTTTTTT AATAGAATAT AATTATCGGA AGCAGTGCCT | |
| | | TCCATAATTA TGACAGTTAT ACTGTCGGTT TTTTTTAAAT | |
| | | AAAAGCAGCA TCTGCTAATA AAACCCAACA GATACTGGAA | |
| | | GTTTTGCATT TATGGTCAAC ACTTAAGGGT TTTAGAAAAC | |
| | | AGCCGTCAGC CAAATGTAAT TGAATAAAGT TGAAGCTAAG | |
| | | ATTTAGAGAT GAATTAAATT TAATTAGGGG TTGCTAAGAA | |
| | | GCGAGCACTG ACCAGATAAG AATGCTGGTT TTCCTAAATG | |
| | | CAGTGAATTG TGACCAAGTT ATAAATCAAT GTCACTTAAA | |
| | | GGCTGTGGTA GTACTCCTGC AAAATTTTAT AGCTCAGTTT | |
| | | ATCCAAGGTG TAACTCTAAT TCCCATTTTG CAAAATTTCC | |
| | | AGTACCTTTG TCACAATCCT AACACATTAT CGGGAGCAGT | |
| | | GTCTTCCATA ATGTATAAAG AACAAGGTAG TTTTTACCTA | |
| | | CCACAGTGTC TGTATCGGAG ACAGTGATCT CCATATGTTA | |
| | | CACTAAGGGT GTAAGTAATT ATCGGGAACA GTGTTTCCCA | |
| | | TAATTTTCTT CATGCAATGA CATCTTCAAA GCTTGAAGAT | |
| | | CGTTAGTATC TAACATGTAT CCCAACTCCT ATAATTCCCT | |
| | | ATCTTTTAGT TTTAGTTGCA GAAACATTTT GTGGTCATTA | |
| | | AGCATTGGGT GGGTAAATTC AACCACTGTA AAATGAAATT | |
| | | ACTACAAAAT TTGAAATTTA GCTTGGGTTT TTGTTACCTT | |
| | | TATGGTTTCT CCAGGTCCTC TACTTAATGA GATAGTAGCA | |
| | | TACATTTATA ATGTTTGCTA TTGACAAGTC ATTTTAACTT | |
| | | TATCACATTA TTTGCATGTT ACCTCCTATA AACTTAGTGC | |
| | | GGACAAGTTT TAATCCAGAA TTGACCTTTT GACTTAAAGC | |
| | | AGAGGGACTT TGTATAGAAG GTTTGGGGGC TGTGGGGAAG | |
| | | GAGAGTCCCC TGAAGGTCTG ACACGTCTGC CTACCCATTC | |
| | | GTGGTGATCA ATTAAATGTA GGTATGAATA AGTTCGAAGC | |
| | | TCCGTGAGTG AACCATCATT ATAAACGTGA TGATCAGCTG | |
| | | TTTGTCATAG GGCAGTTGGA AACGGCCTCC TAGGGAAAAG | |
| | | TTCATAGGGT CTCTTCAGGT TCTTAGTGTC ACTTACCTAG | |
| | | ATTTACAGCC TCACTTGAAT GTGTCACTAC TCACAGTCTC | |
| | | TTTAATCTTC AGTTTTATCT TTAATCTCCT CTTTTATCTT | |
| | | GGACTGACAT TTAGCGTAGC TAAGTGAAAA GGTCATAGCT | |
| | | GAGATTCCTG GTTCGGGTGT TACGCACACG TACTTAAATG | |
| | | AAAGCATGTG GCATGTTCAT CGTATAACAC AATATGAATA | |
| | | CAGGGCATGC ATTTTGCAGC AGTGAGTCTC TTCAGAAAAC | |
| | | CCTTTTCTAC AGTTAGGGTT GAGTTACTTC CTATCAAGCC | |
| | | AGTACGTGCT AACAGGCTCA ATATTCCTGA ATGAAATATC | |
| | | AGACTAGTGA CAAGCTCCTG GTCTTGAGAT GTCTTCTCGT | |
| | | TAAGGAGATG GGCCTTTTGG AGGTAAAGGA TAAAATGAAT | |
| | | GAGTTCTGTC ATGATTCACT ATTCTAGAAC TTGCATGACC | |
| | | TTTACTGTGT TAGCTCTTTG AATGTTCTTG AAATTTTAGA | |
| | | CTTTCTTTGT AAACAAATGA TATGTCCTTA TCATTGTATA | |
| | | AAAGCTGTTA TGTGCAACAG TGTGGAGATT CCTTGTCTGA | |
| | | TTTAATAAAA TACTTAAACA CTGAAAAAAA AAAA | |
| 18S | X03205.1 | TACCTGGTTG ATCCTGCCAG TAGCATATGC TTGTCTCAAA | 46 |
| | | GATTAAGCCA TGCATGTCTA AGTACGCACG GCCGGTACAG | |
| | | TGAAACTGCG AATGGCTCAT TAAATCAGTT ATGGTTCCTT | |
| | | TGGTCGCTCG CTCCTCTCCC ACTTGGATAA CTGTGGTAAT | |
| | | TCTAGAGCTA ATACATGCCG ACGGGCGCTG ACCCCCTTCG | |
| | | CGGGGGGGAT GCGTGCATTT ATCAGATCAA AACCAACCCG | |
| | | GTCAGCCCCT CTCCGGCCCC GGCCGGGGGG CGGGCGCCGG | |
| | | CGGCTTTGGT GACTCTAGAT AACCTCGGGC CGATCGCACG | |
| | | CCCCCCGTGG CGGCGACGAC CCATTCGAAC GTCTGCCCTA | |
| | | TCAACTTTCG ATGGTAGTCG CCGTGCCTAC CATGGTGACC | |
| | | ACGGGTGACG GGGAATCAGG GTTCGATTCC GGAGAGGGAG | |
| | | CCTGAGAAAC GGCTACCACA TCCAAGGAAG GCAGCAGGCG | |
| | | CGCAAATTAC CCACTCCCGA CCCGGGGAGG TAGTGACGAA | |
| | | AAATAACAAT ACAGGACTCT TTCGAGGCCC TGTAATTGGA | |
| | | ATGAGTCCAC TTTAAATCCT TTAACGAGGA TCCATTGGAG | |
| | | GGCAAGTCTG GTGCCAGCAG CCGCGGTAAT TCCAGCTCCA | |
| | | ATAGCGTATA TTAAAGTTGC TGCAGTTAAA AAGCTCGTAG | |
| | | TTGGATCTTG GGAGCGGGCG GGCGGTCCGC CGCGAGGCGA | |
| | | GCCACCGCCC GTCCCCGCCC CTTGCCTCTC GGCGCCCCCT | |
| | | CGATGCTCTT AGCTGAGTGT CCCGCGGGGC CCGAAGCGTT | |
| | | TACTTTGAAA AAATTAGAGT GTTCAAAGCA GGCCCGAGCC | |
| | | GCCTGGATAC CGCAGCTAGG AATAATGGAA TAGGACCGCG | |
| | | GTTCTATTTT GTTGGTTTTC GGAACTGAGG CCATGATTAA | |
| | | GAGGGACGGC CGGGGGCATT CGTATTGCGC CGCTAGAGGT | |
| | | GAAATTCTTG GACCGGCGCA AGACGGACCA GAGCGAAAGC | |
| | | ATTTGCCAAG AATGTTTTCA TTAATCAAGA ACGAAAGTCG | |
| | | GAGGTTCGAA GACGATCAGA TACCGTCGTA GTTCCGACCA | |
| | | TAAACGATGC CGACCGGCGA TGCGGCGGCG TTATTCCCAT | |
| | | GACCCGCCGG GCAGCTTCCG GGAAACCAAA GTCTTTGGGT | |
| | | TCCGGGGGGA GTATGGTTGC AAAGCTGAAA CTTAAAGGAA | |
| | | TTGACGGAAG GGCACCACCA GGAGTGGAGC CTGCGGCTTA | |
| | | ATTTGACTCA ACACGGGAAA CCTCACCCGG CCCGGACACG | |
| | | GACAGGATTG ACAGATTGAT AGCTCTTTCT CGATTCCGTG | |
| | | GGTGGTGGTG CATGGCCGTT CTTAGTTGGT GGAGCGATTT | |
| | | GTCTGGTTAA TTCCGATAAC GAACGAGACT CTGGCATGCT | |
| | | AACTAGTTAC GCGACCCCCG AGCGGTCGGC GTCCCCCAAC | |
| | | TTCTTAGAGG GACAAGTGGC GTTCAGCCAC CCGAGATTGA | |
| | | GCAATAACAG GTCTGTGATG CCCTTAGATG TCCGGGGCTG | |
| | | CACGCGCGCT ACACTGACTG GCTCAGCGTG TGCCTACCCT | |
| | | ACGCCGGCAG GCGCGGGTAA CCCGTTGAAC CCCATTCGTG | |
| | | ATGGGGATCG GGGATTGCAA TTATTCCCCA TGAACGAGGA | |
| | | ATTCCCAGTA AGTGCGGGTC ATAAGCTTGC GTTGATTAAG | |
| | | TCCCTGCCCT TTGTACACAC CGCCCGTCGC TACTACCGAT | |
| | | TGGATGGTTT AGTGAGGCCC TCGGATCGGC CCCGCCGGGG | |
| | | TCGGCCCACG GCCCTGGCGG AGCGCTGAGA AGACGGTCGA | |
| | | ACTTGACTAT CTAGAGGAAG TAAAAGTCGT AACAAGGTTT | |
| | | CCGTAGGTGA ACCTGCGGAA GGATCATTA | |
| PPIA | NM_021130.4 | GGGGCCGAAC GTGGTATAAA AGGGGCGGGA GGCCAGGCTC | 47 |
| | | GTGCCGTTTT GCAGACGCCA CCGCCGAGGA AAACCGTGTA | |
| | | CTATTAGCCA TGGTCAACCC CACCGTGTTC TTCGACATTG | |
| | | CCGTCGACGG CGAGCCCTTG GGCCGCGTCT CCTTTGAGCT | |
| | | GTTTGCAGAC AAGGTCCCAA AGACAGCAGA AAATTTTCGT | |
| | | GCTCTGAGCA CTGGAGAGAA AGGATTTGGT TATAAGGGTT | |
| | | CCTGCTTTCA CAGAATTATT CCAGGGTTTA TGTGTCAGGG | |
| | | TGGTGACTTC ACACGCCATA ATGGCACTGG TGGCAAGTCC | |
| | | ATCTATGGGG AGAAATTTGA AGATGAGAAC TTCATCCTAA | |
| | | AGCATACGGG TCCTGGCATC TTGTCCATGG CAAATGCTGG | |
| | | ACCCAACACA AATGGTTCCC AGTTTTTCAT CTGCACTGCC | |
| | | AAGACTGAGT GGTTGGATGG CAAGCATGTG GTGTTTGGCA | |
| | | AAGTGAAAGA AGGCATGAAT ATTGTGGAGG CCATGGAGCG | |
| | | CTTTGGGTCC AGGAATGGCA AGACCAGCAA GAAGATCACC | |
| | | ATTGCTGACT GTGGACAACT CGAATAAGTT TGACTTGTGT | |
| | | TTTATCTTAA CCACCAGATC ATTCCTTCTG TAGCTCAGGA | |
| | | GAGCACCCCT CCACCCCATT TGCTCGCAGT ATCCTAGAAT | |
| | | CTTTGTGCTC TCGCTGCAGT TCCCTTTGGG TTCCATGTTT | |
| | | TCCTTGTTCC CTCCCATGCC TAGCTGGATT GCAGAGTTAA | |
| | | GTTTATGATT ATGAAATAAA AACTAAATAA CAATTGTCCT | |
| | | CGTTTGAGTT AAGAGTGTTG ATGTAGGCTT TATTTTAAGC | |
| | | AGTAATGGGT TACTTCTGAA ACATCACTTG TTTGCTTAAT | |
| | | TCTACACAGT ACTTAGATTT TTTTTACTTT CCAGTCCCAG | |
| | | GAAGTGTCAA TGTTTGTTGA GTGGAATATT GAAAATGTAG | |
| | | GCAGCAACTG GGCATGGTGG CTCACTGTCT GTAATGTATT | |
| | | ACCTGAGGCA GAAGACCACC TGAGGGTAGG AGTCAAGATC | |
| | | AGCCTGGGCA ACATAGTGAG ACGCTGTCTC TACAAAAAAT | |
| | | AATTAGCCTG GCCTGGTGGT GCATGCCTAG TCCTAGCTGA | |
| | | TCTGGAGGCT GACGTGGGAG GATTGCTTGA GCCTAGAGTG | |
| | | AGCTATTATC ATGCCACTGT ACAGCCTGGG TGTTCACAGA | |
| | | TCTTGTGTCT CAAAGGTAGG CAGAGGCAGG AAAAGCAAGG | |
| | | AGCCAGAATT AAGAGGTTGG GTCAGTCTGC AGTGAGTTCA | |
| | | TGCATTTAGA GGTGTTCTTC AAGATGACTA ATGTCAAAAA | |
| | | TTGAGACATC TGTTGCGGTT TTTTTTTTTT TTTTTTCCCC | |
| | | TGGAATGCAG TGGCGTGATC TCAGCTCACT GCAGCCTCCG | |
| | | CCTCCTGGGT TCAAGTGATT CTAGTGCCTC AGCCTCCTGA | |
| | | GTAGCTGGGA TAATGGGCGT GTGCCACCAT GCCCAGCTAA | |
| | | TTTTTGTATT TTTAGTATAG ATGGGGTTTC ATCATTTTGA | |
| | | CCAGGCTGGT CTCAAACTCT TGACCTCAGC TGATGCGCCT | |
| | | GCCTTGGCCT CCCAAACTGC TGAGATTACA GATGTGAGCC | |
| | | ACCGCACCCT ACCTCATTTT CTGTAACAAA GCTAAGCTTG | |
| | | AACACTGTTG ATGTTCTTGA GGGAAGCATA TTGGGCTTTA | |
| | | GGCTGTAGGT CAAGTTTATA CATCTTAATT ATGGTGGAAT | |
| | | TCCTATGTAG AGTCTAAAAA GCCAGGTACT TGGTGCTACA | |
| | | GTCAGTCTCC CTGCAGAGGG TTAAGGCGCA GACTACCTGC | |
| | | AGTGAGGAGG TACTGCTTGT AGCATATAGA GCCTCTCCCT | |
| | | AGCTTTGGTT ATGGAGGCTT TGAGGTTTTG CAAACCTGAC | |
| | | CAATTTAAGC CATAAGATCT GGTCAAAGGG ATACCCTTCC | |
| | | CACTAAGGAC TTGGTTTCTC AGGAAATTAT ATGTACAGTG | |
| | | CTTGCTGGCA GTTAGATGTC AGGACAATCT AAGCTGAGAA | |
| | | AACCCCTTCT CTGCCCACCT TAACAGACCT CTAGGGTTCT | |
| | | TAACCCAGCA ATCAAGTTTG CCTATCCTAG AGGTGGCGGA | |
| | | TTTGATCATT TGGTGTGTTG GGCAATTTTT GTTTTACTGT | |
| | | CTGGTTCCTT CTGCGTGAAT TACCACCACC ACCACTTGTG | |
| | | CATCTCAGTC TTGTGTGTTG TCTGGTTACG TATTCCCTGG | |
| | | GTGATACCAT TCAATGTCTT AATGTACTTG TGGCTCAGAC | |
| | | CTGAGTGCAA GGTGGAAATA AACATCAAAC ATCTTTTCAT | |
| | | TATCCCTA | |
| PGK1 | NM_000291.3 | GAGAGCAGCG GCCGGGAAGG GGCGGTGCGG GAGGCGGGGT | 48 |
| | | GTGGGGCGGT AGTGTGGGCC CTGTTCCTGC CCGCGCGGTG | |
| | | TTCCGCATTC TGCAAGCCTC CGGAGCGCAC GTCGGCAGTC | |
| | | GGCTCCCTCG TTGACCGAAT CACCGACCTC TCTCCCCAGC | |
| | | TGTATTTCCA AAATGTCGCT TTCTAACAAG CTGACGCTGG | |
| | | ACAAGCTGGA CGTTAAAGGG AAGCGGGTCG TTATGAGAGT | |
| | | CGACTTCAAT GTTCCTATGA AGAACAACCA GATAACAAAC | |
| | | AACCAGAGGA TTAAGGCTGC TGTCCCAAGC ATCAAATTCT | |
| | | GCTTGGACAA TGGAGCCAAG TCGGTAGTCC TTATGAGCCA | |
| | | CCTAGGCCGG CCTGATGGTG TGCCCATGCC TGACAAGTAC | |
| | | TCCTTAGAGC CAGTTGCTGT AGAACTCAAA TCTCTGCTGG | |
| | | GCAAGGATGT TCTGTTCTTG AAGGACTGTG TAGGCCCAGA | |
| | | AGTGGAGAAA GCCTGTGCCA ACCCAGCTGC TGGGTCTGTC | |
| | | ATCCTGCTGG AGAACCTCCG CTTTCATGTG GAGGAAGAAG | |
| | | GGAAGGGAAA AGATGCTTCT GGGAACAAGG TTAAAGCCGA | |
| | | GCCAGCCAAA ATAGAAGCTT TCCGAGCTTC ACTTTCCAAG | |
| | | CTAGGGGATG TCTATGTCAA TGATGCTTTT GGCACTGCTC | |
| | | ACAGAGCCCA CAGCTCCATG GTAGGAGTCA ATCTGCCACA | |
| | | GAAGGCTGGT GGGTTTTTGA TGAAGAAGGA GCTGAACTAC | |
| | | TTTGCAAAGG CCTTGGAGAG CCCAGAGCGA CCCTTCCTGG | |
| | | CCATCCTGGG CGGAGCTAAA GTTGCAGACA AGATCCAGCT | |
| | | CATCAATAAT ATGCTGGACA AAGTCAATGA GATGATTATT | |
| | | GGTGGTGGAA TGGCTTTTAC CTTCCTTAAG GTGCTCAACA | |
| | | ACATGGAGAT TGGCACTTCT CTGTTTGATG AAGAGGGAGC | |
| | | CAAGATTGTC AAAGACCTAA TGTCCAAAGC TGAGAAGAAT | |
| | | GGTGTGAAGA TTACCTTGCC TGTTGACTTT GTCACTGCTG | |
| | | ACAAGTTTGA TGAGAATGCC AAGACTGGCC AAGCCACTGT | |
| | | GGCTTCTGGC ATACCTGCTG GCTGGATGGG CTTGGACTGT | |
| | | GGTCCTGAAA GCAGCAAGAA GTATGCTGAG GCTGTCACTC | |
| | | GGGCTAAGCA GATTGTGTGG AATGGTCCTG TGGGGGTATT | |
| | | TGAATGGGAA GCTTTTGCCC GGGGAACCAA AGCTCTCATG | |
| | | GATGAGGTGG TGAAAGCCAC TTCTAGGGGC TGCATCACCA | |
| | | TCATAGGTGG TGGAGACACT GCCACTTGCT GTGCCAAATG | |
| | | GAACACGGAG GATAAAGTCA GCCATGTGAG CACTGGGGGT | |
| | | GGTGCCAGTT TGGAGCTCCT GGAAGGTAAA GTCCTTCCTG | |
| | | GGGTGGATGC TCTCAGCAAT ATTTAGTACT TTCCTGCCTT | |
| | | TTAGTTCCTG TGCACAGCCC CTAAGTCAAC TTAGCATTTT | |
| | | CTGCATCTCC ACTTGGCATT AGCTAAAACC TTCCATGTCA | |
| | | AGATTCAGCT AGTGGCCAAG AGATGCAGTG CCAGGAACCC | |
| | | TTAAACAGTT GCACAGCATC TCAGCTCATC TTCACTGCAC | |
| | | CCTGGATTTG CATACATTCT TCAAGATCCC ATTTGAATTT | |
| | | TTTAGTGACT AAACCATTGT GCATTCTAGA GTGCATATAT | |
| | | TTATATTTTG CCTGTTAAAA AGAAAGTGAG CAGTGTTAGC | |
| | | TTAGTTCTCT TTTGATGTAG GTTATTATGA TTAGCTTTGT | |
| | | CACTGTTTCA CTACTCAGCA TGGAAACAAG ATGAAATTCC | |
| | | ATTTGTAGGT AGTGAGACAA AATTGATGAT CCATTAAGTA | |
| | | AACAATAAAA GTGTCCATTG AAACCGTGAT TTTTTTTTTT | |
| | | TTCCTGTCAT ACTTTGTTAG GAAGGGTGAG AATAGAATCT | |
| | | TGAGGAACGG ATCAGATGTC TATATTGCTG AATGCAAGAA | |
| | | GTGGGGCAGC AGCAGTGGAG AGATGGGACA ATTAGATAAA | |
| | | TGTCCATTCT TTATCAAGGG CCTACTTTAT GGCAGACATT | |
| | | GTGCTAGTGC TTTTATTCTA ACTTTTATTT TTATCAGTTA | |
| | | CACATGATCA TAATTTAAAA AGTCAAGGCT TATAACAAAA | |
| | | AAGCCCCAGC CCATTCCTCC CATTCAAGAT TCCCACTCCC | |
| | | CAGAGGTGAC CACTTTCAAC TCTTGAGTTT TTCAGGTATA | |
| | | TACCTCCATG TTTCTAAGTA ATATGCTTAT ATTGTTCACT | |
| | | TCTTTTTTTT TTATTTTTTA AAGAAATCTA TTTCATACCA | |
| | | TGGAGGAAGG CTCTGTTCCA CATATATTTC CACTTCTTCA | |
| | | TTCTCTCGGT ATAGTTTTGT CACAATTATA GATTAGATCA | |
| | | AAAGTCTACA TAACTAATAC AGCTGAGCTA TGTAGTATGC | |
| | | TATGATTAAA TTTACTTATG TAAAAAAAAA AAAAAAAAA | |
| RPL13A | NM_012423.3 | CACTTCTGCC GCCCCTGTTT CAAGGGATAA GAAACCCTGC | 49 |
| | | GACAAAACCT CCTCCTTTTC CAAGCGGCTG CCGAAGATGG | |
| | | CGGAGGTGCA GGTCCTGGTG CTTGATGGTC GAGGCCATCT | |
| | | CCTGGGCCGC CTGGCGGCCA TCGTGGCTAA ACAGGTACTG | |
| | | CTGGGCCGGA AGGTGGTGGT CGTACGCTGT GAAGGCATCA | |
| | | ACATTTCTGG CAATTTCTAC AGAAACAAGT TGAAGTACCT | |
| | | GGCTTTCCTC CGCAAGCGGA TGAACACCAA CCCTTCCCGA | |
| | | GGCCCCTACC ACTTCCGGGC CCCCAGCCGC ATCTTCTGGC | |
| | | GGACCGTGCG AGGTATGCTG CCCCACAAAA CCAAGCGAGG | |
| | | CCAGGCCGCT CTGGACCGTC TCAAGGTGTT TGACGGCATC | |
| | | CCACCGCCCT ACGACAAGAA AAAGCGGATG GTGGTTCCTG | |
| | | CTGCCCTCAA GGTCGTGCGT CTGAAGCCTA CAAGAAAGTT | |
| | | TGCCTATCTG GGGCGCCTGG CTCACGAGGT TGGCTGGAAG | |
| | | TACCAGGCAG TGACAGCCAC CCTGGAGGAG AAGAGGAAAG | |
| | | AGAAAGCCAA GATCCACTAC CGGAAGAAGA AACAGCTCAT | |
| | | GAGGCTACGG AAACAGGCCG AGAAGAACGT GGAGAAGAAA | |
| | | ATTGACAAAT ACACAGAGGT CCTCAAGACC CACGGACTCC | |
| | | TGGTCTGAGC CCAATAAAGA CTGTTAATTC CTCATGCGTT | |
| | | GCCTGCCCTT CCTCCATTGT TGCCCTGGAA TGTACGGGAC | |
| | | CCAGGGGCAG CAGCAGTCCA GGTGCCACAG GCAGCCCTGG | |
| | | GACATAGGAA GCTGGGAGCA AGGAAAGGGT CTTAGTCACT | |
| | | GCCTCCCGAA GTTGCTTGAA AGCACTCGGA GAATTGTGCA | |
| | | GGTGTCATTT ATCTATGACC AATAGGAAGA GCAACCAGTT | |
| | | ACTATGAGTG AAAGGGAGCC AGAAGACTGA TTGGAGGGCC | |
| | | CTATCTTGTG AGTGGGGCAT CTGTTGGACT TTCCACCTGG | |
| | | TCATATACTC TGCAGCTGTT AGAATGTGCA AGCACTTGGG | |
| | | GACAGCATGA GCTTGCTGTT GTACACAGGG TATTTCTAGA | |
| | | AGCAGAAATA GACTGGGAAG ATGCACAACC AAGGGGTTAC | |
| | | AGGCATCGCC CATGCTCCTC ACCTGTATTT TGTAATCAGA | |
| | | AATAAATTGC TTTTAAAGAA AAAAAAAAAA AAAAAA | |
| B2M | NM_004048.2 | AATATAAGTG GAGGCGTCGC GCTGGCGGGC ATTCCTGAAG | 50 |
| | | CTGACAGCAT TCGGGCCGAG ATGTCTCGCT CCGTGGCCTT | |
| | | AGCTGTGCTC GCGCTACTCT CTCTTTCTGG CCTGGAGGCT | |
| | | ATCCAGCGTA CTCCAAAGAT TCAGGTTTAC TCACGTCATC | |
| | | CAGCAGAGAA TGGAAAGTCA AATTTCCTGA ATTGCTATGT | |
| | | GTCTGGGTTT CATCCATCCG ACATTGAAGT TGACTTACTG | |
| | | AAGAATGGAG AGAGAATTGA AAAAGTGGAG CATTCAGACT | |
| | | TGTCTTTCAG CAAGGACTGG TCTTTCTATC TCTTGTACTA | |
| | | CACTGAATTC ACCCCCACTG AAAAAGATGA GTATGCCTGC | |
| | | CGTGTGAACC ATGTGACTTT GTCACAGCCC AAGATAGTTA | |
| | | AGTGGGATCG AGACATGTAA GCAGCATCAT GGAGGTTTGA | |
| | | AGATGCCGCA TTTGGATTGG ATGAATTCCA AATTCTGCTT | |
| | | GCTTGCTTTT TAATATTGAT ATGCTTATAC ACTTACACTT | |
| | | TATGCACAAA ATGTAGGGTT ATAATAATGT TAACATGGAC | |
| | | ATGATCTTCT TTATAATTCT ACTTTGAGTG CTGTCTCCAT | |
| | | GTTTGATGTA TCTGAGCAGG TTGCTCCACA GGTAGCTCTA | |
| | | GGAGGGCTGG CAACTTAGAG GTGGGGAGCA GAGAATTCTC | |
| | | TTATCCAACA TCAACATCTT GGTCAGATTT GAACTCTTCA | |
| | | ATCTCTTGCA CTCAAAGCTT GTTAAGATAG TTAAGCGTGC | |
| | | ATAAGTTAAC TTCCAATTTA CATACTCTGC TTAGAATTTG | |
| | | GGGGAAAATT TAGAAATATA ATTGACAGGA TTATTGGAAA | |
| | | TTTGTTATAA TGAATGAAAC ATTTTGTCAT ATAAGATTCA | |
| | | TATTTACTTC TTATACATTT GATAAAGTAA GGCATGGTTG | |
| | | TGGTTAATCT GGTTTATTTT TGTTCCACAA GTTAAATAAA | |
| | | TCATAAAACT TGATGTGTTA TCTCTTA | |
| YWHAZ | NM_003406.3 | CTTTCTCCTT CCCCTTCTTC CGGGCTCCCG TCCCGGCTCA | 51 |
| | | TCACCCGGCC TGTGGCCCAC TCCCACCGCC AGCTGGAACC | |
| | | CTGGGGACTA CGACGTCCCT CAAACCTTGC TTCTAGGAGA | |
| | | TAAAAAGAAC ATCCAGTCAT GGATAAAAAT GAGCTGGTTC | |
| | | AGAAGGCCAA ACTGGCCGAG CAGGCTGAGC GATATGATGA | |
| | | CATGGCAGCC TGCATGAAGT CTGTAACTGA GCAAGGAGCT | |
| | | GAATTATCCA ATGAGGAGAG GAATCTTCTC TCAGTTGCTT | |
| | | ATAAAAATGT TGTAGGAGCC CGTAGGTCAT CTTGGAGGGT | |
| | | CGTCTCAAGT ATTGAACAAA AGACGGAAGG TGCTGAGAAA | |
| | | AAACAGCAGA TGGCTCGAGA ATACAGAGAG AAAATTGAGA | |
| | | CGGAGCTAAG AGATATCTGC AATGATGTAC TGTCTCTTTT | |
| | | GGAAAAGTTC TTGATCCCCA ATGCTTCACA AGCAGAGAGC | |
| | | AAAGTCTTCT ATTTGAAAAT GAAAGGAGAT TACTACCGTT | |
| | | ACTTGGCTGA GGTTGCCGCT GGTGATGACA AGAAAGGGAT | |
| | | TGTCGATCAG TCACAACAAG CATACCAAGA AGCTTTTGAA | |
| | | ATCAGCAAAA AGGAAATGCA ACCAACACAT CCTATCAGAC | |
| | | TGGGTCTGGC CCTTAACTTC TCTGTGTTCT ATTATGAGAT | |
| | | TCTGAACTCC CCAGAGAAAG CCTGCTCTCT TGCAAAGACA | |
| | | GCTTTTGATG AAGCCATTGC TGAACTTGAT ACATTAAGTG | |
| | | AAGAGTCATA CAAAGACAGC ACGCTAATAA TGCAATTACT | |
| | | GAGAGACAAC TTGACATTGT GGACATCGGA TACCCAAGGA | |
| | | GACGAAGCTG AAGCAGGAGA AGGAGGGGAA AATTAACCGG | |
| | | CCTTCCAACT TTTGTCTGCC TCATTCTAAA ATTTACACAG | |
| | | TAGACCATTT GTCATCCATG CTGTCCCACA AATAGTTTTT | |
| | | TGTTTACGAT TTATGACAGG TTTATGTTAC TTCTATTTGA | |
| | | ATTTCTATAT TTCCCATGTG GTTTTTATGT TTAATATTAG | |
| | | GGGAGTAGAG CCAGTTAACA TTTAGGGAGT TATCTGTTTT | |
| | | CATCTTGAGG TGGCCAATAT GGGGATGTGG AATTTTTATA | |
| | | CAAGTTATAA GTGTTTGGCA TAGTACTTTT GGTACATTGT | |
| | | GGCTTCAAAA GGGCCAGTGT AAAACTGCTT CCATGTCTAA | |
| | | GCAAAGAAAA CTGCCTACAT ACTGGTTTGT CCTGGCGGGG | |
| | | AATAAAAGGG ATCATTGGTT CCAGTCACAG GTGTAGTAAT | |
| | | TGTGGGTACT TTAAGGTTTG GAGCACTTAC AAGGCTGTGG | |
| | | TAGAATCATA CCCCATGGAT ACCACATATT AAACCATGTA | |
| | | TATCTGTGGA ATACTCAATG TGTACACCTT TGACTACAGC | |
| | | TGCAGAAGTG TTCCTTTAGA CAAAGTTGTG ACCCATTTTA | |
| | | CTCTGGATAA GGGCAGAAAC GGTTCACATT CCATTATTTG | |
| | | TAAAGTTACC TGCTGTTAGC TTTCATTATT TTTGCTACAC | |
| | | TCATTTTATT TGTATTTAAA TGTTTTAGGC AACCTAAGAA | |
| | | CAAATGTAAA AGTAAAGATG CAGGAAAAAT GAATTGCTTG | |
| | | GTATTCATTA CTTCATGTAT ATCAAGCACA GCAGTAAAAC | |
| | | AAAAACCCAT GTATTTAACT TTTTTTTAGG ATTTTTGCTT | |
| | | TTGTGATTTT TTTTTTTTTG ATACTTGCCT AACATGCATG | |
| | | TGCTGTAAAA ATAGTTAACA GGGAAATAAC TTGAGATGAT | |
| | | GGCTAGCTTT GTTTAATGTC TTATGAAATT TTCATGAACA | |
| | | ATCCAAGCAT AATTGTTAAG AACACGTGTA TTAAATTCAT | |
| | | GTAAGTGGAA TAAAAGTTTT ATGAATGGAC TTTTCAACTA | |
| | | CTTTCTCTAC AGCTTTTCAT GTAAATTAGT CTTGGTTCTG | |
| | | AAACTTCTCT AAAGGAAATT GTACATTTTT TGAAATTTAT | |
| | | TCCTTATTCC CTCTTGGCAG CTAATGGGCT CTTACCAAGT | |
| | | TTAAACACAA AATTTATCAT AACAAAAATA CTACTAATAT | |
| | | AACTACTGTT TCCATGTCCC ATGATCCCCT CTCTTCCTCC | |
| | | CCACCCTGAA AAAAATGAGT TCCTATTTTT TCTGGGAGAG | |
| | | GGGGGGATTG ATTAGAAAAA AATGTAGTGT GTTCCATTTA | |
| | | AAATTTTGGC ATATGGCATT TTCTAACTTA GGAAGCCACA | |
| | | ATGTTCTTGG CCCATCATGA CATTGGGTAG CATTAACTGT | |
| | | AAGTTTTGTG CTTCCAAATC ACTTTTTGGT TTTTAAGAAT | |
| | | TTCTTGATAC TCTTATAGCC TGCCTTCAAT TTTGATCCTT | |
| | | TATTCTTTCT ATTTGTCAGG TGCACAAGAT TACCTTCCTG | |
| | | TTTTAGCCTT CTGTCTTGTC ACCAACCATT CTTACTTGGT | |
| | | GGCCATGTAC TTGGAAAAAG GCCGCATGAT CTTTCTGGCT | |
| | | CCACTCAGTG TCTAAGGCAC CCTGCTTCCT TTGCTTGCAT | |
| | | CCCACAGACT ATTTCCCTCA TCCTATTTAC TGCAGCAAAT | |
| | | CTCTCCTTAG TTGATGAGAC TGTGTTTATC TCCCTTTAAA | |
| | | ACCCTACCTA TCCTGAATGG TCTGTCATTG TCTGCCTTTA | |
| | | AAATCCTTCC TCTTTCTTCC TCCTCTATTC TCTAAATAAT | |
| | | GATGGGGCTA AGTTATACCC AAAGCTCACT TTACAAAATA | |
| | | TTTCCTCAGT ACTTTGCAGA AAACACCAAA CAAAAATGCC | |
| | | ATTTTAAAAA AGGTGTATTT TTTCTTTTAG AATGTAAGCT | |
| | | CCTCAAGAGC AGGGACAATG TTTTCTGTAT GTTCTATTGT | |
| | | GCCTAGTACA CTGTAAATGC TCAATAAATA TTGATGATGG | |
| | | GAGGCAGTGA GTCTTGATGA TAAGGGTGAG AAACTGAAAT | |
| | | CCCAAACACT GTTTTGTTGC TTGTTTTATT ATGACCTCAG | |
| | | ATTAAATTGG GAAATATTGG CCCTTTTGAA TAATTGTCCC | |
| | | AAATATTACA TTCAAATAAA AGTGCAATGG AGAAAAAAAA | |
| | | AAA | |
| SDHA | NM_004168.3 | ACTGCAGCCC CGCTCGACTC CGGCGTGGTG CGCAGGCGCG | 52 |
| | | GTATCCCCCC TCCCCCGCCA GCTCGACCCC GGTGTGGTGC | |
| | | GCAGGCGCAG TCTGCGCAGG GACTGGCGGG ACTGCGCGGC | |
| | | GGCAACAGCA GACATGTCGG GGGTCCGGGG CCTGTCGCGG | |
| | | CTGCTGAGCG CTCGGCGCCT GGCGCTGGCC AAGGCGTGGC | |
| | | CAACAGTGTT GCAAACAGGA ACCCGAGGTT TTCACTTCAC | |
| | | TGTTGATGGG AACAAGAGGG CATCTGCTAA AGTTTCAGAT | |
| | | TCCATTTCTG CTCAGTATCC AGTAGTGGAT CATGAATTTG | |
| | | ATGCAGTGGT GGTAGGCGCT GGAGGGGCAG GCTTGCGAGC | |
| | | TGCATTTGGC CTTTCTGAGG CAGGGTTTAA TACAGCATGT | |
| | | GTTACCAAGC TGTTTCCTAC CAGGTCACAC ACTGTTGCAG | |
| | | CACAGGGAGG AATCAATGCT GCTCTGGGGA ACATGGAGGA | |
| | | GGACAACTGG AGGTGGCATT TCTACGACAC CGTGAAGGGC | |
| | | TCCGACTGGC TGGGGGACCA GGATGCCATC CACTACATGA | |
| | | CGGAGCAGGC CCCCGCCGCC GTGGTCGAGC TAGAAAATTA | |
| | | TGGCATGCCG TTTAGCAGAA CTGAAGATGG GAAGATTTAT | |
| | | CAGCGTGCAT TTGGTGGACA GAGCCTCAAG TTTGGAAAGG | |
| | | GCGGGCAGGC CCATCGGTGC TGCTGTGTGG CTGATCGGAC | |
| | | TGGCCACTCG CTATTGCACA CCTTATATGG AAGGTCTCTG | |
| | | CGATATGATA CCAGCTATTT TGTGGAGTAT TTTGCCTTGG | |
| | | ATCTCCTGAT GGAGAATGGG GAGTGCCGTG GTGTCATCGC | |
| | | ACTGTGCATA GAGGACGGGT CCATCCATCG CATAAGAGCA | |
| | | AAGAACACTG TTGTTGCCAC AGGAGGCTAC GGGCGCACCT | |
| | | ACTTCAGCTG CACGTCTGCC CACACCAGCA CTGGCGACGG | |
| | | CACGGCCATG ATCACCAGGG CAGGCCTTCC TTGCCAGGAC | |
| | | CTAGAGTTTG TTCAGTTCCA CCCTACAGGC ATATATGGTG | |
| | | CTGGTTGTCT CATTACGGAA GGATGTCGTG GAGAGGGAGG | |
| | | CATTCTCATT AACAGTCAAG GCGAAAGGTT TATGGAGCGA | |
| | | TACGCCCCTG TCGCGAAGGA CCTGGCGTCT AGAGATGTGG | |
| | | TGTCTCGGTC CATGACTCTG GAGATCCGAG AAGGAAGAGG | |
| | | CTGTGGCCCT GAGAAAGATC ACGTCTACCT GCAGCTGCAC | |
| | | CACCTACCTC CAGAGCAGCT GGCCACGCGC CTGCCTGGCA | |
| | | TTTCAGAGAC AGCCATGATC TTCGCTGGCG TGGACGTCAC | |
| | | GAAGGAGCCG ATCCCTGTCC TCCCCACCGT GCATTATAAC | |
| | | ATGGGCGGCA TTCCCACCAA CTACAAGGGG CAGGTCCTGA | |
| | | GGCACGTGAA TGGCCAGGAT CAGATTGTGC CCGGCCTGTA | |
| | | CGCCTGTGGG GAGGCCGCCT GTGCCTCGGT ACATGGTGCC | |
| | | AACCGCCTCG GGGCAAACTC GCTCTTGGAC CTGGTTGTCT | |
| | | TTGGTCGGGC ATGTGCCCTG AGCATCGAAG AGTCATGCAG | |
| | | GCCTGGAGAT AAAGTCCCTC CAATTAAACC AAACGCTGGG | |
| | | GAAGAATCTG TCATGAATCT TGACAAATTG AGATTTGCTG | |
| | | ATGGAAGCAT AAGAACATCG GAACTGCGAC TCAGCATGCA | |
| | | GAAGTCAATG CAAAATCATG CTGCCGTGTT CCGTGTGGGA | |
| | | AGCGTGTTGC AAGAAGGTTG TGGGAAAATC AGCAAGCTCT | |
| | | ATGGAGACCT AAAGCACCTG AAGACGTTCG ACCGGGGAAT | |
| | | GGTCTGGAAC ACGGACCTGG TGGAGACCCT GGAGCTGCAG | |
| | | AACCTGATGC TGTGTGCGCT GCAGACCATC TACGGAGCAG | |
| | | AGGCACGGAA GGAGTCACGG GGCGCGCATG CCAGGGAAGA | |
| | | CTACAAGGTG CGGATTGATG AGTACGATTA CTCCAAGCCC | |
| | | ATCCAGGGGC AACAGAAGAA GCCCTTTGAG GAGCACTGGA | |
| | | GGAAGCACAC CCTGTCCTAT GTGGACGTTG GCACTGGGAA | |
| | | GGTCACTCTG GAATATAGAC CCGTGATCGA CAAAACTTTG | |
| | | AACGAGGCTG ACTGTGCCAC CGTCCCGCCA GCCATTCGCT | |
| | | CCTACTGATG AGACAAGATG TGGTGATGAC AGAATCAGCT | |
| | | TTTGTAATTA TGTATAATAG CTCATGCATG TGTCCATGTC | |
| | | ATAACTGTCT TCATACGCTT CTGCACTCTG GGGAAGAAGG | |
| | | AGTACATTGA AGGGAGATTG GCACCTAGTG GCTGGGAGCT | |
| | | TGCCAGGAAC CCAGTGGCCA GGGAGCGTGG CACTTACCTT | |
| | | TGTCCCTTGC TTCATTCTTG TGAGATGATA AAACTGGGCA | |
| | | CAGCTCTTAA ATAAAATATA AATGAACAAA CTTTCTTTTA | |
| | | TTTCCAAATC CATTTGAAAT ATTTTACTGT TGTGACTTTA | |
| | | GTCATATTTG TTGACCTAAA AATCAAATGT AATCTTTGTA | |
| | | TTGTGTTACA TCAAAATCCA GATATTTTGT ATAGTTTCTT | |
| | | TTTTCTTTTT CTTTTCTTTT TTTTTTTTGA GACAGGATCG | |
| | | GTGCAGTAGT ACAATCACAG CTCACTGCAG CCTCAAACTC | |
| | | CTGGGCAGCT CAGGTGATCT TCCTGACTCA GCCTTCTGAG | |
| | | TAGTTGGGGC TACAGGTGTG CACCACCATG CCCAGCTCAT | |
| | | TTATTTTGTA ATTGTAGGGA CAGGGTCTCA CTGTGTTGCC | |
| | | TAGGCTGGTC TCAAGTGATC CTCCCTCCTT GGCCTCCCAA | |
| | | GGTGCTGGAA TTATAGGTGT GAACAAACCA AAAAAAAAAA | |
| | | AAA | |
| HPRT1 | NM_000194.2 | GGCGGGGCCT GCTTCTCCTC AGCTTCAGGC GGCTGCGACG | 53 |
| | | AGCCCTCAGG CGAACCTCTC GGCTTTCCCG CGCGGCGCCG | |
| | | CCTCTTGCTG CGCCTCCGCC TCCTCCTCTG CTCCGCCACC | |
| | | GGCTTCCTCC TCCTGAGCAG TCAGCCCGCG CGCCGGCCGG | |
| | | CTCCGTTATG GCGACCCGCA GCCCTGGCGT CGTGATTAGT | |
| | | GATGATGAAC CAGGTTATGA CCTTGATTTA TTTTGCATAC | |
| | | CTAATCATTA TGCTGAGGAT TTGGAAAGGG TGTTTATTCC | |
| | | TCATGGACTA ATTATGGACA GGACTGAACG TCTTGCTCGA | |
| | | GATGTGATGA AGGAGATGGG AGGCCATCAC ATTGTAGCCC | |
| | | TCTGTGTGCT CAAGGGGGGC TATAAATTCT TTGCTGACCT | |
| | | GCTGGATTAC ATCAAAGCAC TGAATAGAAA TAGTGATAGA | |
| | | TCCATTCCTA TGACTGTAGA TTTTATCAGA CTGAAGAGCT | |
| | | ATTGTAATGA CCAGTCAACA GGGGACATAA AAGTAATTGG | |
| | | TGGAGATGAT CTCTCAACTT TAACTGGAAA GAATGTCTTG | |
| | | ATTGTGGAAG ATATAATTGA CACTGGCAAA ACAATGCAGA | |
| | | CTTTGCTTTC CTTGGTCAGG CAGTATAATC CAAAGATGGT | |
| | | CAAGGTCGCA AGCTTGCTGG TGAAAAGGAC CCCACGAAGT | |
| | | GTTGGATATA AGCCAGACTT TGTTGGATTT GAAATTCCAG | |
| | | ACAAGTTTGT TGTAGGATAT GCCCTTGACT ATAATGAATA | |
| | | CTTCAGGGAT TTGAATCATG TTTGTGTCAT TAGTGAAACT | |
| | | GGAAAAGCAA AATACAAAGC CTAAGATGAG AGTTCAAGTT | |
| | | GAGTTTGGAA ACATCTGGAG TCCTATTGAC ATCGCCAGTA | |
| | | AAATTATCAA TGTTCTAGTT CTGTGGCCAT CTGCTTAGTA | |
| | | GAGCTTTTTG CATGTATCTT CTAAGAATTT TATCTGTTTT | |
| | | GTACTTTAGA AATGTCAGTT GCTGCATTCC TAAACTGTTT | |
| | | ATTTGCACTA TGAGCCTATA GACTATCAGT TCCCTTTGGG | |
| | | CGGATTGTTG TTTAACTTGT AAATGAAAAA ATTCTCTTAA | |
| | | ACCACAGCAC TATTGAGTGA AACATTGAAC TCATATCTGT | |
| | | AAGAAATAAA GAGAAGATAT ATTAGTTTTT TAATTGGTAT | |
| | | TTTAATTTTT ATATATGCAG GAAAGAATAG AAGTGATTGA | |
| | | ATATTGTTAA TTATACCACC GTGTGTTAGA AAAGTAAGAA | |
| | | GCAGTCAATT TTCACATCAA AGACAGCATC TAAGAAGTTT | |
| | | TGTTCTGTCC TGGAATTATT TTAGTAGTGT TTCAGTAATG | |
| | | TTGACTGTAT TTTCCAACTT GTTCAAATTA TTACCAGTGA | |
| | | ATCTTTGTCA GCAGTTCCCT TTTAAATGCA AATCAATAAA | |
| | | TTCCCAAAAA TTTAAAAAAA AAAAAAAAAA AAAAA | |
| TOX4 | NM_001303523.1 | AGCAGAGAGA ACACACGTCC TTGCGGAAGT GACGGCAGTT | 54 |
| | | CCGAGTCCAG TGGGGGCGGT GGGAGCGATG AGGGTCTGAG | |
| | | ACGGTGGGAG CGGTTGTGTG AAGATGGAGA CATTCCATAC | |
| | | ACCAAGCTTG GGTGATGAGG AATTTGAAAT CCCACCTATC | |
| | | TCCTTGGATT CTGATCCCTC ATTGGCTGTC TCAGATGTGG | |
| | | TTGGCCACTT TGATGACCTG GCAGACCCTT CCTCTTCACA | |
| | | GGATGGCAGT TTTTCAGCCC AGTATGGGGT CCAGACATTG | |
| | | GACATGCCTG TGGGCATGAC CCATGGCTTG ATGGAGCAGG | |
| | | GCGGGGGGCT CCTGAGTGGG GGCTTGACCA TGGACTTGGA | |
| | | CCACTCTATA GGAACTCAGT ATAGTGCCAA CCCACCTGTT | |
| | | ACAATTGATG TACCAATGAC AGACATGACA TCTGGCTTGA | |
| | | TGGGGCATAG CCAGTTGACC ACCATTGATC AGTCAGAACT | |
| | | GAGTTCCCAG CTGGGTTTGA GCCTAGGGGG TGGCACCATC | |
| | | CTGCCACCTG CCCAGTCACC TGAAGATCGT CTTTCAACCA | |
| | | CCCCTTCACC TACTAGTTCA CTTCACGAGG ATGGTGTTGA | |
| | | GGATTTCCGG AGGCAACTTC CCAGCCAGAA GACAGTCGTG | |
| | | GTGGAAGCAG GGAAAAAGCA GAAGGCCCCA AAGAAGAGAA | |
| | | AAAAGAAAGA TCCTAATGAA CCTCAGAAAC CAGTTTCAGC | |
| | | ATATGCTTTA TTCTTTCGTG ATACACAGGC TGCCATCAAG | |
| | | GGACAGAATC CTAATGCCAC TTTTGGTGAG GTTTCAAAAA | |
| | | TTGTGGCCTC CATGTGGGAT AGTCTTGGAG AGGAGCAAAA | |
| | | ACAGGTATAT AAGAGGAAAA CTGAGGCTGC CAAGAAAGAG | |
| | | TATCTGAAGG CACTGGCTGC TTACAAAGAC AACCAGGAGT | |
| | | GTCAGGCCAC TGTGGAAACA GTGGAATTGG ATCCAGCACC | |
| | | ACCATCACAA ACTCCTTCTC CACCTCCTAT GGCTACTGTT | |
| | | GACCCAGCAT CTCCAGCACC AGCTTCAATA GAGCCCCCTG | |
| | | CCCTGTCCCC ATCCATTGTT GTTAACTCCA CCCTTTCATC | |
| | | CTATGTGGCA AACCAGGCAT CTTCTGGAGC TGGGGGTCAG | |
| | | CCCAATATCA CCAAGTTGAT TATTACCAAA CAAATGTTGC | |
| | | CCTCTTCTAT TACTATGTCT CAAGGAGGGA TGGTTACTGT | |
| | | TATCCCAGCC ACAGTGGTGA CCTCCCGGGG GCTCCAACTA | |
| | | GGCCAAACCA GTACAGCTAC TATCCAGCCC AGTCAACAAG | |
| | | CCCAGATTGT CACTCGGTCA GTGTTGCAGG CAGCAGCAGC | |
| | | TGCTGCTGCT GCTGCTTCTA TGCAACTGCC TCCACCCCGA | |
| | | CTACAGCCCC CTCCATTACA ACAGATGCCA CAGCCCCCGA | |
| | | CTCAGCAGCA AGTTACCATT CTGCAGCAGC CTCCTCCACT | |
| | | CCAGGCCATG CAACAGCCTC CACCTCAGAA AGTTCGAATC | |
| | | AATTTACAGC AACAGCCTCC TCCTCTGCAG ATCAAGAGTG | |
| | | TGCCTCTACC CACTTTGAAA ATGCAGACTA CCTTAGTCCC | |
| | | ACCAACTGTG GAAAGTAGTC CTGAGCGGCC TATGAACAAC | |
| | | AGCCCTGAGG CCCATACAGT GGAGGCACCT TCTCCTGAGA | |
| | | CTATCTGTGA GATGATCACA GATGTAGTTC CTGAGGTTGA | |
| | | GTCTCCTTCT CAGATGGATG TTGAATTGGT GAGTGGGTCT | |
| | | CCTGTGGCAC TCTCACCCCA GCCTCGATGT GTGAGGTCTG | |
| | | GTTGTGAGAA CCCTCCCATT GTGAGTAAGG ACTGGGACAA | |
| | | TGAATACTGC AGCAATGAGT GTGTGGTGAA GCACTGCAGG | |
| | | GATGTATTCT TGGCCTGGGT AGCCTCTAGA AATTCAAACA | |
| | | CAGTGGTGTT TGTGAAATAG TCCTTCCTGT TCTCCAAGCC | |
| | | AGTGAAGAGT TATCTGCTGG GAAAGTGTCC AAGAGCCTGT | |
| | | TTTTGAAACA CAAGCTGGGC TTCTGGTAGT GCCTCATCAC | |
| | | AACCCATGAT GGCTGTTCAT GTTTCACCCC TTTTCTTCCT | |
| | | TCAGCAGAGG CCAGGCTATG GAGCAGGGCC ACTGAATTTG | |
| | | CTGTAATCTG GAGATGCTTT TTACTTTCAA CCATAAGCGG | |
| | | TAATAGCAGA GGAAAGGGTG AAGGGAGTCT GGGCAAGCAA | |
| | | AGCATAGAGA TGGTGGGGTG GTGGTGGGGT TGAAGAAACT | |
| | | TGTTGGTATA ATTGTCATAG GACTTGCCTA AAATATTATT | |
| | | AAAATTACGG GAGTGTACTC AGCTTTGAGC CTAGGAGAAA | |
| | | ATGCCACTGT GTGCATCCAT TTTAAAGGGT TCCCTCATAA | |
| | | AAAAATGTTA TTCCCCATTA TCACATCAGT ACACTGCTTT | |
| | | GAAAACAAAA CTTTTCAACA TGGGCATACT GGGCTACATG | |
| | | GAAAATGACA TCACCCAGGA GTGATTTCTC TTTATATATA | |
| | | TTATTTCTGC AGTTACCATC CTTATCTGAG TTATCACAGT | |
| | | TCATGAATCT AAGAGGCGGA ACTCTACATC ATTAGTAAGA | |
| | | GGTTCCACCA AAGTCTAAAG TTGTATTCAC TTGTGTTTGA | |
| | | TGAACTATCT TTAAAAGACC ATAGGTCTAT CATTATTTCT | |
| | | TAGACATAAT CTAAAGAAAA ACAGACTAGA GAAGCCACCT | |
| | | GGTTGTAACA GAATAAGCAG AAGTTTACAG CATGATAGTC | |
| | | CAAGTGGTGA TAACTTTAAA TAAAACTCAA ATTTTTACTG | |
| | | TTTGTAGACA GGAATGCTGT CCTAGAGAAC CTCCTCCTCA | |
| | | ACCAGCTACG TACATAGTTT TATCCTATGC ATTCCTGTTT | |
| | | TCTGTGTGTT TTTTGTTTTT TTTTTTTTTT TTTTTTTTTG | |
| | | AGACAGAGTC TCGCTCTGTC ACCCAGGCTG GAGTGCAGTG | |
| | | GTGCGACCTC AGCTCACTGA AACCTCTGCC TCCCGGGTTC | |
| | | AAGCGATTCT CCTGCATCAG CCTCCCGAGT AGCTAGGATT | |
| | | ACAGGCGCCC GCCACTACGC CCAGCTAATT TGTGGTATTT | |
| | | TTAGTAGAGA CAGGGTTTCA CCATGTTGGC CAGGCTGGTC | |
| | | TCGAACTCCT GACCTCATGA TCCGCCCGCC TTGACCTCCC | |
| | | AAAGTGCTGG GATTACAGGC ATGAGCCACC GCACCCAGCC | |
| | | TGCATTCCTG TTTTTTTAAT GGTTTTGGAG GGTAGCAGTA | |
| | | GAGATGGGGT CTCACTATGT TGCCCAGTCT AGTCTTGAAC | |
| | | TCCTGGGCTA CAGTTACCCT CCTACCTCGG CTTCCCAAAG | |
| | | TGCTCGGATT ACAGGTGTGA GCCACTGTGC CTAGCCTATA | |
| | | ATGATCATTT TAATGTTTCC CATGCACTCA TTTAGTTTGA | |
| | | ACCTTCACAG CAACCCAATG AGGTAATACT CCCATTTCAC | |
| | | ATATAATACT GAGAGATGAG TTGCACAAGA TTATACACTG | |
| | | TTAAGTAGCA GAGCCAGAAT GGACTTCAGA ATCCCAACTA | |
| | | CAATACAAAT GTTTATTTAA ATAAAGAAGA AAGCTATTGT | |
| | | ACAAATATCA CTCTTCAGGT TTAGCTTACA GAGCCATGGC | |
| | | TATGGATTCT TAGCTCTGTA AGGAAGTGCT TCTATAAATT | |
| | | CTTAGGTTTA GAGATGATAC CATCTGGGTA CCTTTGCTTG | |
| | | AACCGTGCAA CCACATCTGG GTCTAGTAGG TGGATCCCAT | |
| | | CCAGTTGGTT TCCAAGGGTG ATCCTGAAAC AGTGTAAAAG | |
| | | GAGGGGCAAA CCAGAAATCC TGGAATTAGA GGGTTTAATA | |
| | | TTGTTAAAAA ATGCATACCA AATGAAGACT GCCTATCATC | |
| | | ATATCAAATA TGCCAATTCT AAAAAGAGCT TAACATTAGA | |
| | | ATAGTATATG GTAGAATTAC TAGTTCAGAA TTGGCATAGA | |
| | | TTCTGGTGTT AAAATAGACT GGATCTGTAT TATCTGAGGG | |
| | | TTAGTAACTA ATGCTTAGCC AGGCCTGCTT CACAGAGTTG | |
| | | CTACCAGGGA GTATTCTTTG GATAAGCAAA ATGCTAGCAG | |
| | | CATGTGTTTT AAGCTCTGTT AAGGGGTGAA AGATGTAATT | |
| | | ATTGACAGAT TAAATAGATA ACTTCGTAAC CACCAGGGGG | |
| | | CAGATTCAAT ACATCACAGA ATGGCTGAGG AAGATCCTTG | |
| | | GGTTGTGAAG AGAGTAGAAA CCCTAGGGAG CAGTGCTTTT | |
| | | GGGTCCTAGA ACCTGTTGAG TTTCTAATGA ATATTTGTAG | |
| | | AATCTCATAA AACAGTTTAA ATACAAGCTT AAGTGGCTTA | |
| | | TGAATCCTGT GAAGCTCATT TATGGACTAG TGTAAAACAA | |
| | | TGTGAAGCTC TACTAAGTTC TGTCCTTAAT CATAAATAAT | |
| | | AGCCCCTTGA GGACTAGCCT GTTCTCTGGT CACCTTACCA | |
| | | GTTGGGTTGC ACATTGTGTG GTCGTCCAAA TAACTCAATC | |
| | | TTGCGAGTGC CAGGAGATAG TCTTTCAATC ATGCCATAGA | |
| | | TTTCATCTGG TTTATGACTG GTGGAACGAA CCTAGGAAAT | |
| | | AAAAACTAGC TGCTTTTTAA GTTACACAAG AAAAAA | |

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

As used herein, the terms “polynucleotide” and “nucleic acid molecule” are used interchangeably to mean a polymeric form of nucleotides of at least 10 bases or base pairs in length, either ribonucleotides or deoxynucleotides or a modified form of either type of nucleotide, and is meant to include single and double stranded forms of DNA. As used herein, a nucleic acid molecule or nucleic acid sequence that serves as a probe in a microarray analysis preferably comprises a chain of nucleotides, more preferably DNA and/or RNA. In other embodiments, a nucleic acid molecule or nucleic acid sequence comprises other kinds of nucleic acid structures such as for instance a DNA/RNA helix, peptide nucleic acid (PNA), locked nucleic acid (LNA) and/or a ribozyme. Hence, as used herein the term “nucleic acid molecule” also encompasses a chain comprising non-natural nucleotides, modified nucleotides and/or non-nucleotide building blocks which exhibit the same function as natural nucleotides.

As used herein, the terms “hybridize,” “hybridizing”, “hybridizes,” and the like, used in the context of polynucleotides, are meant to refer to conventional hybridization conditions, such as hybridization in 50% formamide/6×SSC/0.1% SDS/100 μg/ml ssDNA, in which temperatures for hybridization are above 37 degrees centigrade and temperatures for washing in 0.1×SSC/0.1% SDS are above 55 degrees C., and preferably to stringent hybridization conditions.

As used herein, the term “normalization” or “normalizer” refers to the expression of a differential value in terms of a standard value to adjust for effects which arise from technical variation due to sample handling, sample preparation, and measurement methods rather than biological variation of biomarker concentration in a sample. For example, when measuring the expression of a differentially expressed protein, the absolute value for the expression of the protein can be expressed in terms of an absolute value for the expression of a standard protein that is substantially constant in expression. In some embodiments, normalizing the expression level of a gene to the expression level of a housekeeping gene means dividing the expression level of the gene by the expression level of the housekeeping gene.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore, the term diagnosis includes: a. prediction (determining if a patient will likely develop aggressive disease (hyperproliferative/invasive)), b. prognosis (predicting whether a patient will likely have a better or worse outcome at a pre-selected time in the future), c. therapy selection, d. therapeutic drug monitoring, and e. relapse monitoring.

The term “providing” as used herein with regard to a biological sample refers to directly or indirectly obtaining the biological sample from a subject. For example, “providing” may refer to the act of directly obtaining the biological sample from a subject (e.g., by a blood draw, tissue biopsy, lavage and the like). Likewise, “providing” may refer to the act of indirectly obtaining the biological sample. For example, providing may refer to the act of a laboratory receiving the sample from the party that directly obtained the sample, or to the act of obtaining the sample from an archive.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

The term “biological sample” as used herein refers to any sample of biological origin potentially containing one or more biomarkers. Examples of biological samples include tissue, organs, or bodily fluids such as whole blood, plasma, serum, tissue, lavage or any other specimen used for detection of disease.

The term “subject” as used herein refers to a mammal, preferably a human.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

Biomarker levels may change due to treatment of the disease. The changes in biomarker levels may be measured by the present disclosure. Changes in biomarker levels may be used to monitor the progression of disease or therapy.

“Altered”, “changed” or “significantly different” refer to a detectable change or difference from a reasonably comparable state, profile, measurement, or the like. Such changes may be all or none. They may be incremental and need not be linear. They may be by orders of magnitude. A change may be an increase or decrease by 1%, 5%, 10%, 20%, 30%, 40%, 50%, 60%, 70%, 80%, 90%, 95%, 99%, 100%, or more, or any value in between 0% and 100%. Alternatively, the change may be 1-fold, 1.5-fold, 2-fold, 3-fold, 4-fold, 5-fold or more, or any values in between 1-fold and five-fold. The change may be statistically significant with a p value of 0.1, 0.05, 0.001, or 0.0001.

The term “stable disease” refers to a diagnosis for the presence of a plasma cell dyscrasia, however the myeloma has been treated and remains in a stable condition, i.e. one that that is not progressive, as determined by imaging data and/or best clinical judgment.

The term “progressive disease” refers to a diagnosis for the presence of a highly active state of a plasma cell dyscrasia, i.e. one has not been treated and is not stable or has been treated and has not responded to therapy, or has been treated and active disease remains, as determined by imaging data and/or best clinical judgment.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Derivation of a 32-Marker Gene Panel

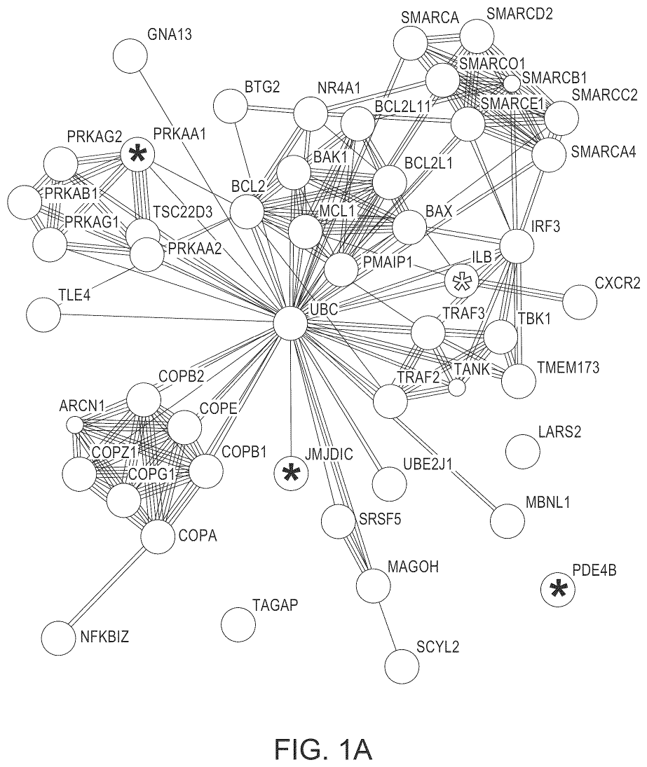

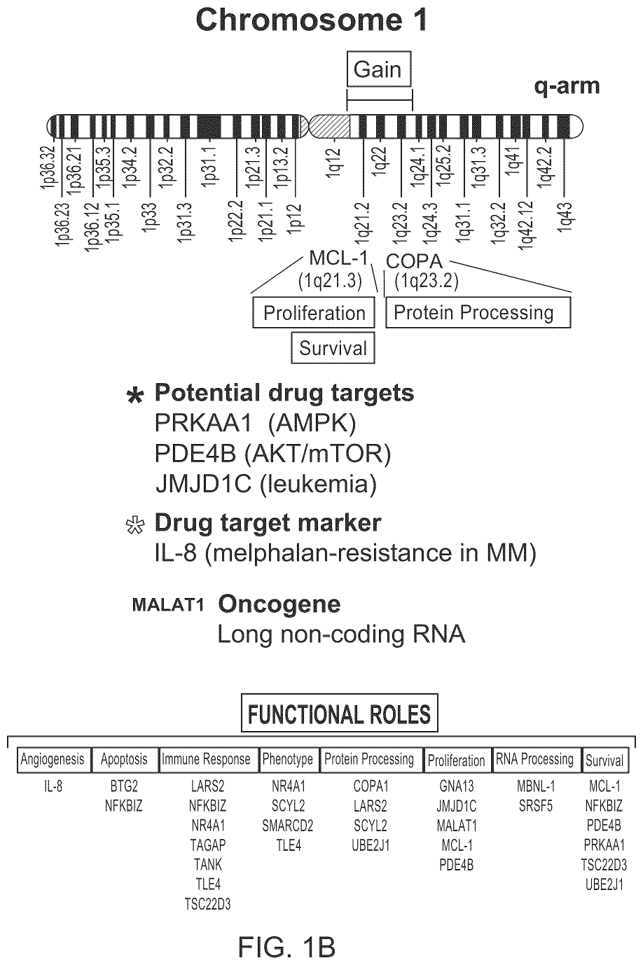

Raw probe intensities (n=1,354,896 features) from n=15 peripheral blood mononuclear cell samples were used to identify genes that best discriminated between controls and multiple myeloma using the transcriptional profile of GSE7116. Following removal of outliers, a total of 31 target transcripts (26 genes, 5 splice variants) and one house-keeping gene was identified in an unbiased manner as potential markers of myeloma (Table 2; FIG. 1A and FIG. 1B). These genes were demonstrated to be expressed in multiple myeloma cell lines, IM-9 (normal diploid karyotype) and MMIR (dexamethasone-resistant), identifying they are produced by transformed B-lymphoblasts.

Then an artificial intelligence model of myeloma disease dynamics was built using normalized gene expression of these 31 markers (normalized to the housekeeping gene, TPT1) in whole blood from Controls (n=45), Responders/Stable (n=24: stable disease), and Newly Diagnosed/Relapsed (n=66: progressive disease) samples. The dataset was randomly split into training and testing partitions for model creation and validation respectively. Twelve algorithms were evaluated (XGB, RF, glmnet, cforest, CART, treebag, knn, nnet, SVM-radial, SVM-linear, NB and mlp) with accuracies ranging from 0.7-0.85). The top performing algorithm (XGB-“gradient boosting”) best predicted the training data. In the test set, XGB produced probability scores that predicted the sample. Each probability score reflects the “certainty” of an algorithm that an unknown sample belongs to either “Stable Disease” or “Progressive” class. For example, an unknown sample S1 can have the following probability vector [Control=0.2, Progressive=0.8]. This sample would be considered a myeloma sample that exhibited progressive disease, given a score of 0.8. If the sample came from a patient with MRD or who was under treatment, the score would identify either they are exhibiting progressive disease (will relapse) or are failing the therapy. MyelomX scores>0.2 are considered indicative the sample is from a myeloma patient.

TABLE 2

| Multiple Myeloma Biomarker or | | | | | | | |
|---|---|---|---|---|---|---|---|
| Housekeeping Genes | | NCBI Chromosome | UniGene | | Amplicon | Exon | Assay |
| Symbol | Name | location | ID | RefSeq | length | Boundary | Location |
| ASXL1 | additional sex combs like 1, | Chr.20: | Hs.374043 | NM_001164603.1 | 95 | 1-2 | 484 |
| | transcriptional regulator | 32358062-32439319 | | | | | |
| BHLHE40 | Basic helix-loop-helix family, | Chr.3: | Hs.744856 | NM_003670.2 | 66 | 4-5 | 683 |
| | member e40 | 4979412-4985181 | | | | | |
| BTG2 | BTG anti-proliferation factor 2 | Chr.1: | Hs.519162 | NM_006763.2 | 86 | 1-2 | 215 |
| | | 203305536-203309602 | | | | | |
| COPA | coatomer protein complex, | Chr.1: | Hs.162121 | NM_001098398.1 | 82 | 6-7 | 892 |
| | subunit alpha | 160288587-160343564 | | | | | |
| FBXW7 | F-box and WD repeat domain | Chr.4: | Hs.561245 | NM_001013415.1 | 76 | 3-4 | 548 |
| | containing 7, E3 ubiquitin | 152321258-152536095 | | | | | |
| | protein ligase | | | | | | |
| GNA13 | Guanine nucleotide binding | Chr.17: | Hs.515018 | NM_006572.5 | 70 | 1-2 | 496 |
| | protein (G protein), alpha 13 | 65009289-65056802 | | | | | |
| IL8 | Interleukin 8 | Chr.4: | Hs.624 | NM_000584.3 | 101 | 1-2 | 222 |
| | | 74606223-74609433 | | | | | |
| JMJD1C | Jumonji domain containing 1C | Chr.10: | Hs.413416 | NM_001282948.1 | 115 | 21-22 | 7309 |
| | | 63167221-63522075 | | | | | |
| LARS2 | leucyl-tRNA synthetase 2, | Chr.3: | Hs.526975 | NM_015340.3 | 83 | 2-3 | 164 |
| | mitochondrial | 45388561-45548842 | | | | | |
| MALAT1 | metastasis associated lung | Chr.11: | Hs.621695 | NR_002819.2 | 117 | 1-1 | 4952 |
| | adenocarcinoma transcript 1 | 65497679-65504494 | | | | | |
| | (non-protein coding) | | | | | | |
| MBNL1 | muscleblind-like splicing | Chr.3: | Hs.201858 | NM_001314057.1 | 55 | 4-5 | 981 |
| | regulator | 152243656-152465780 | | | | | |
| MCL1 | myeloid cell leukemia sequence | Chr.1: | Hs.632486 | NM_001197320.1 | 89 | 3-4 | 682 |
| | 1 (BCL2-related) | 150574551-150579738 | | | | | |
| NFKBIZ | nuclear factor of kappa light | Chr.3: | Hs.319171 | NM_001005474.2 | 62 and | 6-7 and | 1322 and |
| | polypeptide gene enhancer in | 101827990-101861025 | | | 86 | 12-13 | 2087 |
| | B-cells inhibitor, zeta | | | | | | |
| NR4A1 | nuclear receptor subfamily 4, | Chr.12: | Hs.524430 | NM_001202233.1 | 79 and | 3-4 and | 1107 and |
| | group A, member 1 | 52022832-52059507 | | | 87 | 7-8 | 1767 |
| PDE4B | phosphodiesterase 4B, | Chr.1: | Hs.198072 | NM_001037339.2 | 72 | 8-9 | 1429 |
| | cAMP-specific | 65792510-66374579 | | | | | |
| P1AS2 | protein inhibitor of | Chr:18: | Hs.57769 | NM_001324046.1 | 72 | 12-13 | 1716 |
| | activated STAT, 2 | 46803224-46920167 | | | | | |
| PRKAA1 | protein kinase, AMP-activated, | Chr.5: | Hs.43322 | NM_006251.5 | 93 and | 2-3 and | 275 and |
| | alpha 1 catalytic subunit | 40759379-40798195 | | | 75 | 5-6 | 606 |
| SCYL2 | SCY1-like 2 (S. cerevisiae) | Chr.12: | Hs.506481 | NM_001317784.1 | 151 and | 2-3 and | 463 and |
| | | 100267137-100341724 | | | 67 | 8-9 | 1381 |
| SMARCD2 | SWI/SNF related, matrix | Chr.17: | Hs.250581 | NM_001098426.1 | 63 | 4-5 | 885 |
| | associated, actin | 63832081-63842991 | | | | | |
| | dependent regulator of | | | | | | |
| | chromatin, subfamily | | | | | | |
| | d, member 2 | | | | | | |
| SP1 | Sp1 transcription factor | Chr.12: | Hs.620754 | NM_001251825.1 | 62 and | 3-4 and | 1631 and |
| | | 53380195-53416446 | | | 72 | 5-6 | 2002 |
| SRSF5 | serine/arginine-rich splicing | Chr.14: | Hs.632326 | NM_001039465.1 | 102 | 5-6 | 651 |
| | factor 5 | 69767087-69772005 | | | | | |
| TAGAP | T-cell activation RhoGTPase | Chr.6: | Hs.529984 | NM_001278733.1 | 99 | 3-4 | 552 |
| | activating protein | 159034468-159045152 | | | | | |
| TANK | TRAF family member-associated | Chr.2: | Hs.132257 | NM_001199135.1 | 89 | 2-3 | 210 |
| | NFKB activator | 161136955-161236176 | | | | | |
| TLE4 | transducin-like enhancer of | Chr.9: | Hs.444213 | NM_001282748.1 | 89 | 2-3 | 1162 |
| | split 4 (E(sp1) homolog, | 79571773-79726882 | | | | | |
| | Drosophila | | | | | | |
| TPT1 | tumor protein, translationally- | Chr.13: | Hs.374596 | NM_001286272.1 | 131 | 3-3 | 377 |
| (housekeeping | controlled 1 | 45333471-45341284 | | | | | |
| gene) | | | | | | | |
| TSC22D3 | TSC22 domain family, member 3 | Chr.X: | Hs.522074 | NM_001015881.1 | 77 | 2-3 | 220 |
| | | 107713221-107777329 | | | | | |
| UBE2J1 | Ubiquitin-conjugating | Chr.6: | Hs.163776 | NM_016021.2 | 61 | 3-4 | 568 |
| | enzyme E2, J1 | 89326625-89352917 | | | | | |

### Example 2. Diagnosis: Identification of Samples as Myeloma

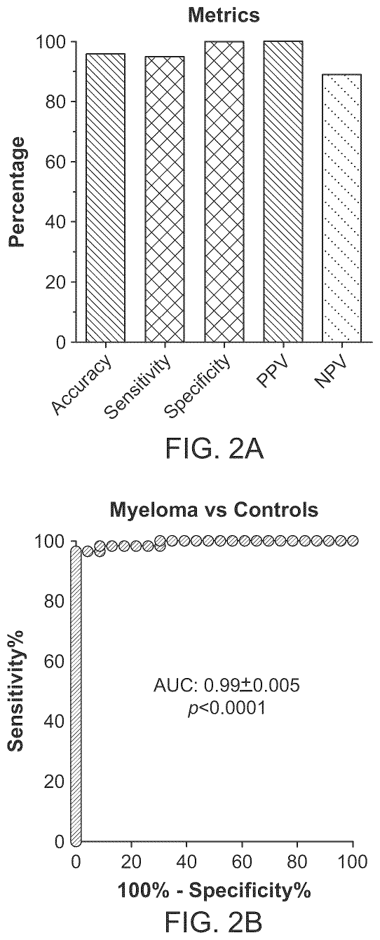

In the test set 1, the data for the utility of the test to differentiate patients with active myeloma disease (n=57) from controls (n=23) are included in Table 3. The receiver operator cuver analysis and metrics are included in FIG. 2B. The score exhibited an area under the curve (AUC) of 0.99. The metrics are: sensitivity>95%, specificity 100%, PPV 100%, NPV 88.5%. The overall accuracy is 96%. The tool can therefore differentiate between controls and aggressive and stable myeloma disease.

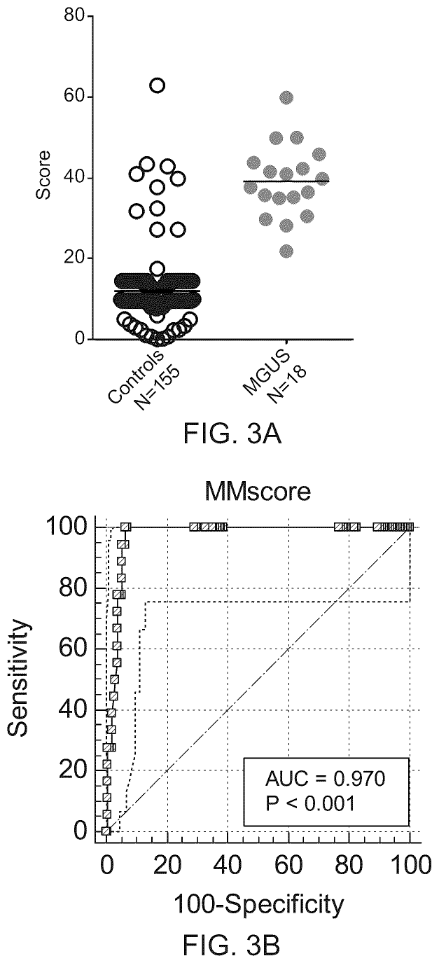

Specific evaluation of the MGUS identified significant differences between this plasma cell dyscrasia (n=18; MyelomX=39±9) and controls (n=155, 12±8, p<0.0001) (FIG. 3A). The AUC for differentiating MGUS from controls 0.97 (FIG. 3B).

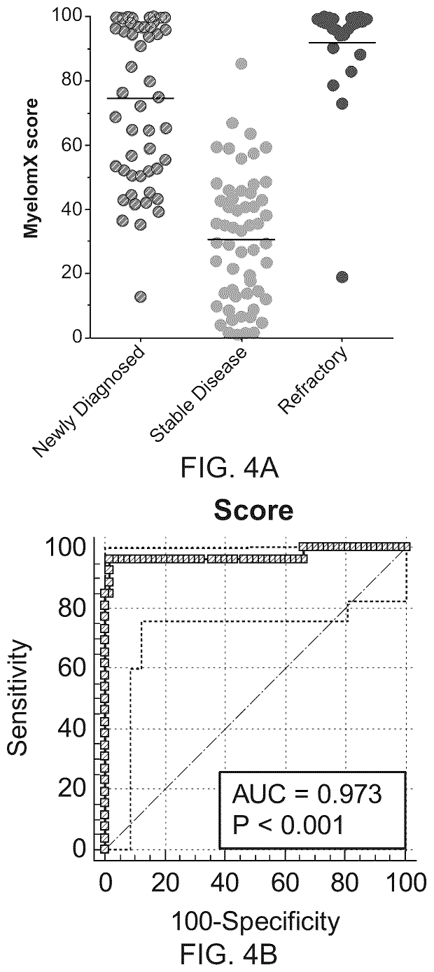

Specific evaluation of the multiple myeloma sub-groups identified significant differences between newly diagnosed patients (n=53; MyelomX=75±25), clinically stable disease (n=56, 31±20, p<0.0001) and refractory disease (n=26, 92±17) (FIG. 4A). The AUC for differentiating stable from refractory disease was 0.97 (FIG. 4B).

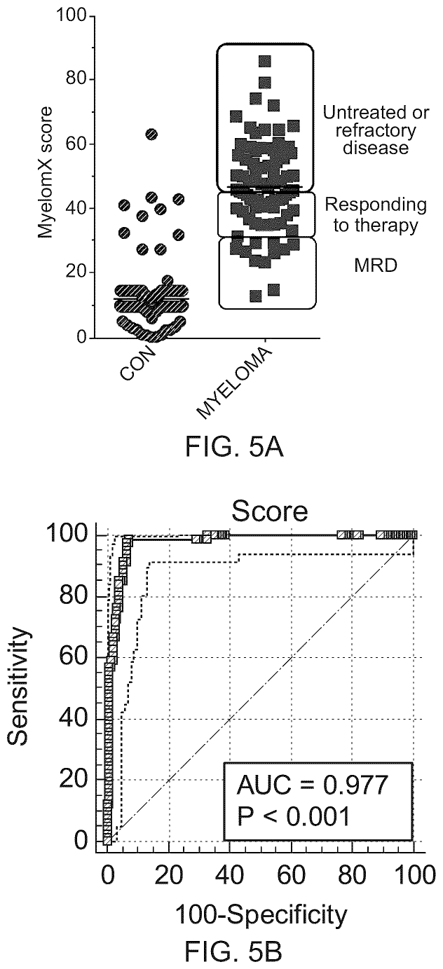

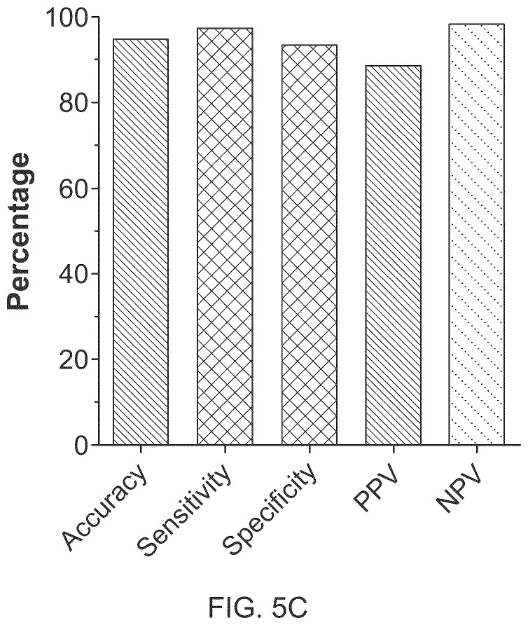

The test was evaluated in a second test set (test set 2) that included 155 healthy controls and 81 myeloma patients, the majority of whom exhibited stable disease including those with MRD. The mean MyelomX score in this myeloma group was 47±14 versus 12±8 in the control group (FIG. 5A). The receiver operator cuver analysis demonstrated the score exhibited an area under the curve (AUC) of 0.97 (FIG. 5B) and the metrics were 89-99% (FIG. 5C).

TABLE 3
Confusion matrix showing classification accuracy of
the XGB-algorithm that determines whether a sample is
a myeloma or a control in blood samples

Reference

| Predicted | Myeloma | Control |
|---|---|---|
| Control | 3 | 20 |
| Myeloma | 55 | 2 |

| | |
|---|---|
| Sensitivity | 96% |
| Specificity | 87% |
| Positive Predictive | 95% |
| Value | |
| Negative Predictive | 81% |
| Value | |
| Accuracy | 94% |

### Example 3. Identification of Minimal Residual Disease

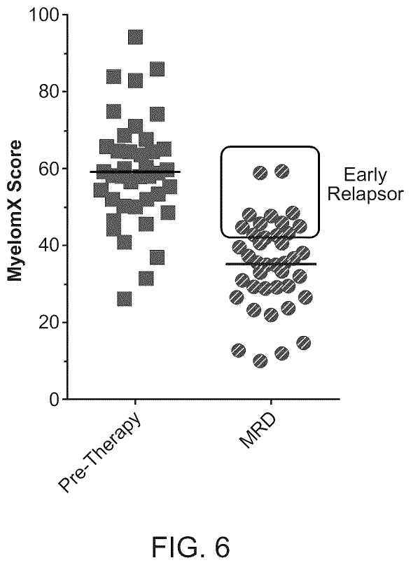

Effective therapy (n=40), decreased the score from 59±14 to 35±12 which was associated with complete remissions (FIG. 6). Evaluation of the MRD group identified ten patients all with high scores (>40) who relapsed at an early time point (within one year). The MyelomX score can therefore biochemically define MRD and identify those who have progressive disease and will relapse at an early time-point.

### Example 4. Differentiating Treatment Responders from Refractory Disease

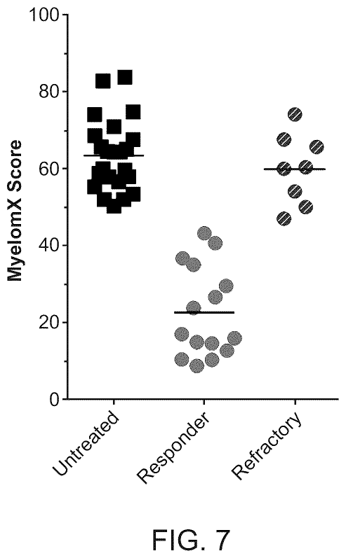

In the therapy series (n=23), treatment with bortemozib (a proteasome inhibitor-PI) for 3 months significantly decreased the MyelomX scores from 64±9 to 23±12 (p<0.0001) in treatment responders but was unchanged in those refractory to PI-treatment (60±9, p=NS) (FIG. 7). This indicates that the MyelomX score can be used to measure the efficacy of proteasome inhibitor therapy in myeloma, that a decrease in score correlates with response to therapeutic intervention and non-responders can accurately be identified.

### Example 5. Use of MyelomX Score to Diagnose Myeloma, Demonstrate MRD and Define Therapy Response

A confusion matrix identifying the accuracy of the MyelomX score in both data sets is included in Table 4. As a diagnostic, the score is 97% accurate for identifying active disease. For determining MRD it is overall 75% accurate, but is 100% accurate for those who do not recur within one year. For treatment responders, the score is 87% accurate for identifying responders and 97% for those who are failing therapy or are refractory.

TABLE 4
Confusion matrix showing classification accuracy of the
XGB-model algorithm and score cut-offs for defining the
dynamic state of myeloma disease in blood samples

| | Reference | | | |
|---|---|---|---|---|
| | Control | Intermediate | MMx | |
| | (MMx Score | MMx Score | Score | |
| Predicted | ≤20) | 21 to 39 | ≥40 | Accuracy |
| Control (n = 47) | 45 | 2 | 0 | 96% |
| Multiple myeloma | 2 | 13 | 50 | 97% |
| (No MRD) (n = 65) | | | | |
| New Diagnosed | 1 | 1 | 29 | 97% |
| (n = 31) | | | | |
| MRD (n = 24) | 2 | 16 | 6 | 75% |
| Treatment | 26 | 8 | 5 | 87% |
| Responder (n = 39) | | | | |
| Refractory Disease | 1 | 0 | 33 | 97% |
| (n = 34) | | | | |

We confirmed that myeloma was the source for the blood-based gene expression assay by evaluating expression in different myeloma cell lines and in FAC-sorted multiple myeloma tumors form patients.

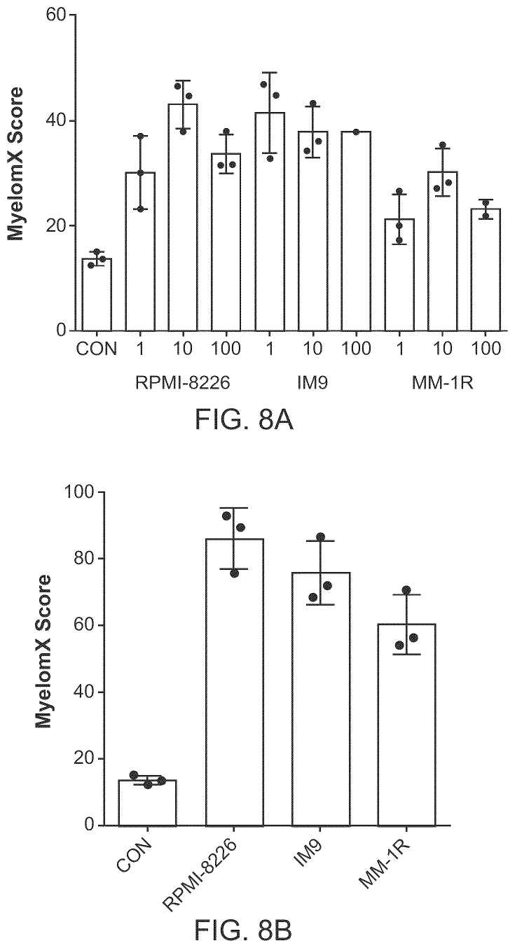

All 32 genes were highly expressed in all 3 myeloma cell lines. Scores ranged from 86±9 (RPMI-8226) to 76±10 (IM9) to 60±9 (MM-1R) (FIG. 8A).

Spike-in experiments using these 3 cell lines and normal whole blood demonstrated that gene expression scores were detected when as few as 1 cell was spiked into 1 ml of blood. One single myleoma cell was detectable. Scores ranged from 30±7 (RPMI-8226) to 41±7 (IM9) to 21±3 (MM-1R) (FIG. 8B). Scores were significantly elevated compared to control blood (no spike-in; p<0.0001).

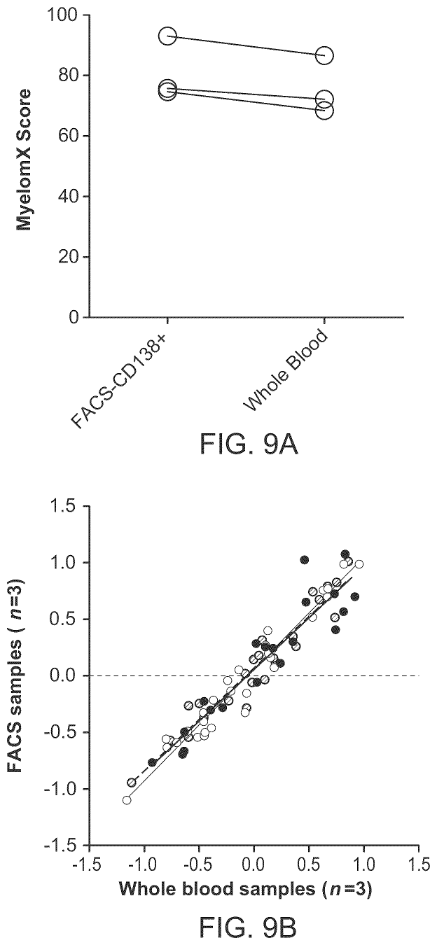

We then evaluated gene expression in tumor tissue (FAC sorted CD138+ cells). All 32 genes (and variants) were highly expressed in myeloma and scores were consistent with values from whole blood samples (FIG. 9A). We also compared gene expression in tumor tissue and matched whole blood from the same patient. Gene expression was highly concordant (Pearson r: 0.87-0.95, median: 0.92) identifying gene expression in tumor tissue and blood was concordant (FIG. 9B).

### REFERENCES

1. Swerdlow S H, Campo E, Pileri S A, et al. The 2016 revision of the World Health Organization classification of lymphoid neoplasms. Blood. 2016; 127:2375-2390. doi: 2310.1182/blood-2016-2301-643569. Epub 64216 March 643515.
2. Siegel R L, Miller K D, Jemal A. Cancer Statistics, 2017. CA Cancer J Clin. 2017; 67:7-30. doi: 10.3322/caac.21387. Epub 2217 January 21385.
3. Michels T C, Petersen K E. Multiple Myeloma: Diagnosis and Treatment. Am Fam Physician. 2017; 95:373-383.
4. Egan P, Drain S, Conway C, Bjourson A J, Alexander H D. Towards Stratified Medicine in Plasma Cell Myeloma. Int J Mol Sci. 2016; 17 (10). E1760.
5. Debes-Marun C S, Dewald G W, Bryant S, et al. Chromosome abnormalities clustering and its implications for pathogenesis and prognosis in myeloma. Leukemia. 2003; 17:427-436.
6. Kyle R A, Rajkumar S V. Multiple myeloma. Blood. 2008; 111:2962-2972. doi: 2910.1182/blood-2007-2910-078022.
7. Melchor L, Brioli A, Wardell C P, et al. Single-cell genetic analysis reveals the composition of initiating clones and phylogenetic patterns of branching and parallel evolution in myeloma. Leukemia. 2014; 28:1705-1715. doi: 1710.1038/leu.2014.1713. Epub 214 January 1713.
8. Fonseca R, Barlogie B, Bataille R, et al. Genetics and cytogenetics of multiple myeloma: a workshop report. Cancer Res. 2004; 64:1546-1558.
9. Gonzalez D, van der Burg M, Garcia-Sanz R, et al. Immunoglobulin gene rearrangements and the pathogenesis of multiple myeloma. Blood. 2007; 110:3112-3121. Epub 27 July 3118.
10. Avet-Loiseau H, Gerson F, Magrangeas F, Minvielle S, Harousseau J L, Bataille R. Rearrangements of the c-myc oncogene are present in 15% of primary human multiple myeloma tumors. Blood. 2001; 98:3082-3086.
11. Walker B A, Wardell C P, Murison A, et al. APOBEC family mutational signatures are associated with poor prognosis translocations in multiple myeloma. Nat Commun. 2015; 6:6997: 10.1038/ncomms7997.
12. Pawlyn C, Melchor L, Murison A, et al. Coexistent hyperdiploidy does not abrogate poor prognosis in myeloma with adverse cytogenetics and may precede IGH translocations. Blood. 2015; 125:831-840. doi: 810.1182/blood-2014-1107-584268. Epub 58214 November 584226.
13. Dimopoulos M, Kyle R, Fermand J P, et al. Consensus recommendations for standard investigative workup: report of the International Myeloma Workshop Consensus Panel 3. Blood. 2011; 117:4701-4705. doi: 4710.1182/blood-2010-4710-299529. Epub 29211 February 299523.
14. Shaughnessy J D, Jr., Zhan F, Burington B E, et al. A validated gene expression model of high-risk multiple myeloma is defined by deregulated expression of genes mapping to chromosome 1. Blood. 2007; 109:2276-2284. Epub 26 Nov. 2214.
15. Chng W J, Braggio E, Mulligan G, et al. The centrosome index is a powerful prognostic marker in myeloma and identifies a cohort of patients that might benefit from aurora kinase inhibition. Blood. 2008; 111:1603-1609. Epub 27 Nov. 1615.
16. Decaux O, Lode L, Magrangeas F, et al. Prediction of survival in multiple myeloma based on gene expression profiles reveals cell cycle and chromosomal instability signatures in high-risk patients and hyperdiploid signatures in low-risk patients: a study of the Intergroupe Francophone du Myelome. J Clin Oncol. 2008; 26:4798-4805. doi: 4710.1200/JCO.2007.4713.8545. Epub 28 June 4730.
17. Dickens N J, Walker B A, Leone P E, et al. Homozygous deletion mapping in myeloma samples identifies genes and an expression signature relevant to pathogenesis and outcome. Clin Cancer Res. 2010; 16:1856-1864. doi: 1810.1158/1078-0432.CCR-1809-2831. Epub 210 March 1859.
18. Hose D, Reme T, Hielscher T, et al. Proliferation is a central independent prognostic factor and target for personalized and risk-adapted treatment in multiple myeloma. Haematologica. 2011; 96:87-95. doi: 10.3324/haematol.2010.030296. Epub 03210 September 030230.
19. Moreaux J, Klein B, Bataille R, et al. A high-risk signature for patients with multiple myeloma established from the molecular classification of human myeloma cell lines. Haematologica. 2011; 96:574-582. doi: 510.3324/haematol.2010.033456. Epub 03210 December 033420.
20. Kuiper R, Broyl A, de Knegt Y, et al. A gene expression signature for high-risk multiple myeloma. Leukemia. 2012; 26:2406-2413. doi: 2410.1038/leu.2012.2127. Epub 2012 May 2408.
21. Chung T H, Mulligan G, Fonseca R, Chng W J. A novel measure of chromosome instability can account for prognostic difference in multiple myeloma. PLOS One. 2013; 8: e66361. doi: 66310.61371/journal.pone.0066361. Print 0062013.
22. Wu P, Walker B A, Broyl A, et al. A gene expression based predictor for high risk myeloma treated with intensive therapy and autologous stem cell rescue. Leuk Lymphoma. 2015; 56:594-601. doi: 510.3109/10428194.10422014.10911863. Epub 1042214 August 10428119.
23. Hermansen N E, Borup R, Andersen M K, et al. Gene expression risk signatures maintain prognostic power in multiple myeloma despite microarray probe set translation. Int J Lab Hematol. 2016; 38:298-307. doi: 210.1111/ijlh. 12486. Epub 1216 March 12429.
24. Chng W J, Chung T H, Kumar S, et al. Gene signature combinations improve prognostic stratification of multiple myeloma patients. Leukemia. 2016; 30:1071-1078. doi: 1010.1038/leu.2015.1341. Epub 215 December 1016.
25. Kuiper R, van Duin M, van Vliet M H, et al. Prediction of high- and low-risk multiple myeloma based on gene expression and the International Staging System. Blood. 2015; 126:1996-2004. doi: 1910.1182/blood-2015-1905-644039. Epub 64215 September 644031.
26. Amin S B, Yip W K, Minvielle S, et al. Gene expression profile alone is inadequate in predicting complete response in multiple myeloma. Leukemia. 2014; 28:2229-2234. doi: 2210.1038/leu.2014.2140. Epub 214 April 2215.
27. Hanahan D, Weinberg R A. The hallmarks of cancer. Cell. 2000; 100:57-70.
28. Hanahan D, Weinberg R A. Hallmarks of cancer: the next generation. Cell. 2011; 144:646-674. doi: 610.1016/j.cell.2011.1002.1013.
29. Walker B A, Boyle E M, Wardell C P, et al. Mutational Spectrum, Copy Number Changes, and Outcome: Results of a Sequencing Study of Patients With Newly Diagnosed Myeloma. J Clin Oncol. 2015; 33:3911-3920. doi: 3910.1200/JCO.2014.3959.1503. Epub 215 August 3917.
30. Kidd M, Drozdov I, Modlin I. Blood and tissue neuroendocrine tumor gene cluster analysis correlate, define hallmarks and predict disease status. Endocr Relat Cancer. 2015; 22:561-575. doi: 510.1530/ERC-1515-0092. Epub 215 June 1532.
31. Li S C, Essaghir A, Martijn C, et al. Global microRNA profiling of well-differentiated small intestinal neuroendocrine tumors. Mod Pathol. 2013; 26:685-696. doi: 610.1038/modpathol.2012.1216. Epub 213 January 1018.
32. Modlin I, Drozdov I, Kidd M. The Identification of gut neuroendocrine tumor disease by multiple synchronous transcript analysis in blood. Plos One. 2013; e63364.
33. Bodei L, Kidd M, Modlin I M, et al. Measurement of circulating transcripts and gene cluster analysis predicts and defines therapeutic efficacy of peptide receptor radionuclide therapy (PRRT) in neuroendocrine tumors. Eur J Nucl Med Mol Imaging. 2016; 43:839-851. doi: 810.1007/s00259-00015-03250-z. Epub 0215 November 00223.
34. Munshi N C, Anderson K C. New strategies in the treatment of multiple myeloma. Clin Cancer Res. 2013; 19:3337-3344. doi: 3310.1158/1078-0432.CCR-3312-1881. Epub 213 March 3320.
35. Raje N, Woo S B, Hande K, et al. Clinical, radiographic, and biochemical characterization of multiple myeloma patients with osteonecrosis of the jaw. Clin Cancer Res. 2008; 14:2387-2395. doi: 2310.1158/1078-0432.CCR-2307-1430.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A kit comprising at least one of: 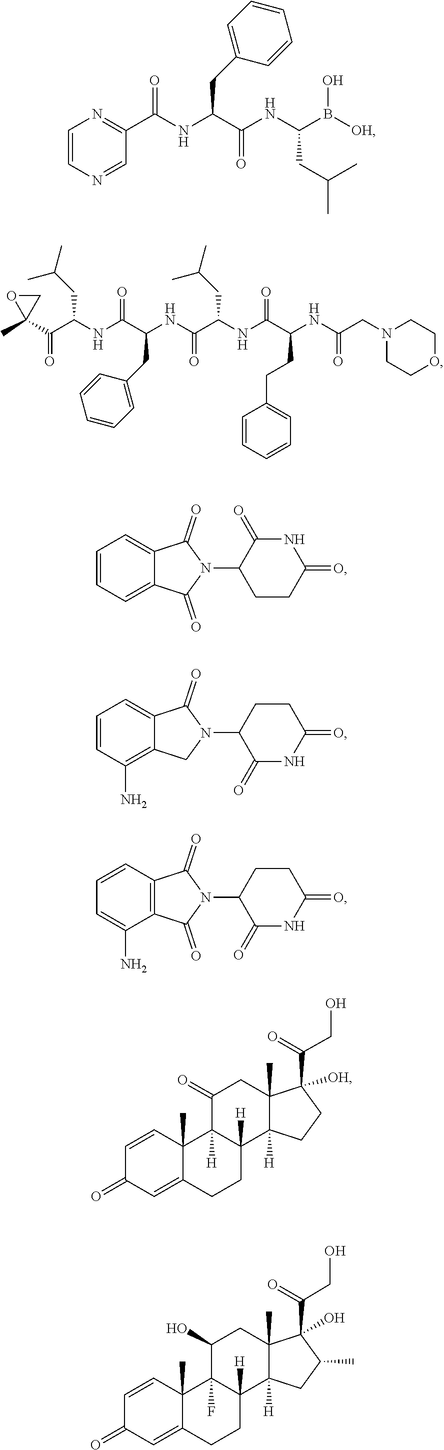
or any combination thereof, and
at least one species of probe that hybridizes to at least one of ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J.

2. The kit of claim 1, wherein the kit comprises:

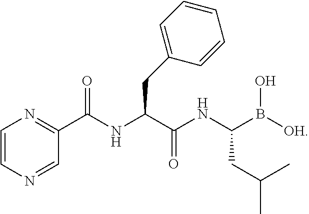 [SMILES: CC(C)C[C@H](NC(=O)[C@H](Cc1ccccc1)NC(=O)c1cnccn1)B(O)O]

3. The kit of claim 1, wherein the kit comprises:

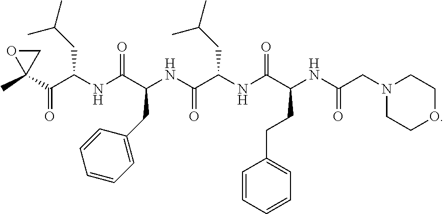 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

4. The kit of claim 1, wherein the kit comprises:

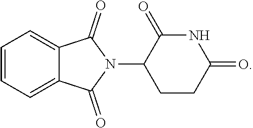 [SMILES: O=C1CCC(N2C(=O)c3ccccc3C2=O)C(=O)N1]

5. The kit of claim 1, wherein the kit comprises:

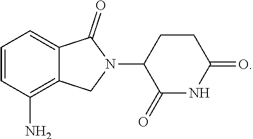 [SMILES: Nc1cccc2c1CN(C1CCC(=O)NC1=O)C2=O]

6. The kit of claim 1, wherein the kit comprises:

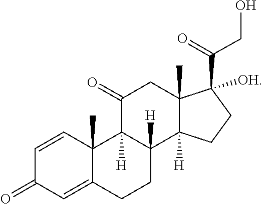

7. The kit of claim 1, wherein the kit comprises:

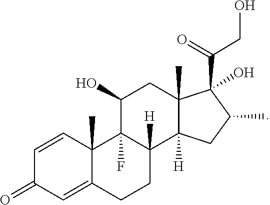

8. The kit of claim 1, wherein the at least one probe further comprises at least one detectable label.

9. The kit of claim 1, wherein the kit comprises at least 28 species of probe, wherein the at least 31 species of probe bind to each of ASXL1, BHLHE40, BTG2, COPA, FBXW7, GNA13, IL8, JMJD1C, LARS2, MALAT1, MBNL1, MCL1, NFKBIZ (2 splice variants), NR4A1 (2 splice variants), PDE4B, PIAS2, PRKAA1 (2 splice variants), SCYL2 (2 splice variants), SMARCD2, SP1 (2 splice variants), SRSF5, TAGAP, TANK, TLE4, TSC22D3, UBE2J.

10. The kit of claim 1, wherein the kit further comprises at least one species of probe that binds to at least one housekeeping gene.

11. The kit of claim 10, wherein the at least one housekeeping gene is selected from ALG9, SEPN, YWHAQ, VPS37A, PRRC2B, DOPEY2, NDUFB11, ND4, MRPL19, PSMC4, SF3A1, PUM1, ACTB, GAPD, GUSB, RPLP0, TFRC, MORF4L1, 18S, PPIA, PGK1, RPL13A, B2M, YWHAZ, SDHA, HPRT1, TOX4, and TPT1.

12. A method of treating cancer in a subject in need thereof, the method comprising administering to the subject at least one therapeutically effective amount of at least one of: 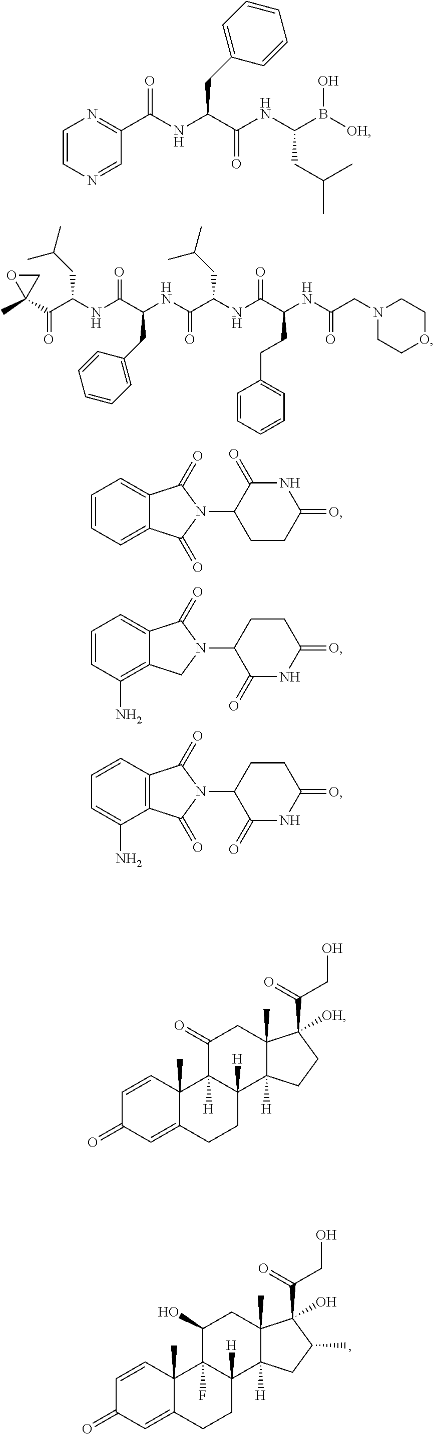
targeted therapy, biological therapy, chemotherapy, corticosteroids, stem cell transplantation, radiation therapy, or any combination thereof.

13. The method of claim 12, wherein the method comprises administering at least one therapeutically effective amount of:

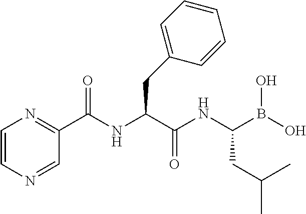 [SMILES: CC(C)C[C@H](NC(=O)[C@H](Cc1ccccc1)NC(=O)c1cnccn1)B(O)O]

14. The method of claim 12, wherein the method comprises administering at least one therapeutically effective amount of:

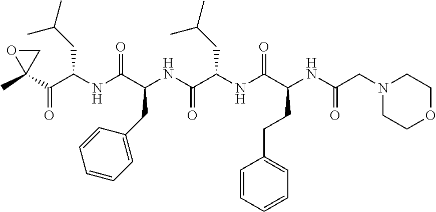 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

15. The method of claim 12, wherein the method comprises administering at least one therapeutically effective amount of:

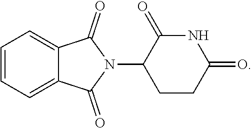 [SMILES: O=C1CCC(N2C(=O)c3ccccc3C2=O)C(=O)N1]

16. The method of claim 12, wherein the method comprises administering at least one therapeutically effective amount of:

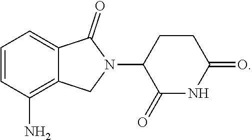 [SMILES: Nc1cccc2c1CN(C1CCC(=O)NC1=O)C2=O]

17. The method of claim 12, wherein the method comprises administering at least one therapeutically effective amount of:

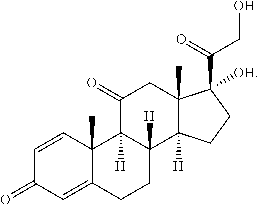

18. The method of claim 12, wherein the method comprises administering at least one therapeutically effective amount of: [chemical structure]


SEQUENCE LISTING (54 sequences)

SEQ ID NO: 1 - DNA, 1084 bp, Homo sapiens
  source 1..1084: mol_type = other DNA
cacacccacggcagacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtattagaaaactactcggatgctccaatgacaccaaaacagattctgcaggtcatagaggcagaaggactaaaggaaatgagaagtgggacttcccctctcgcatgcctcaatgctatgctacattccaattcaagaggaggagaggggttgttttataaactgcctggccgaatcagccttttcacgctcaaggtgtgagccactgcaccaggccccttcatcttaattttaatatatctttgaataaacaccattgtatgaacctgctgtaagcttgggagtggtctgttagtctacagcttgtgtctgagatgtgctaattgaatatttgctcagtacctcatcttaactgcctttggctttatgttgcttatccttcatagtatcttgttcattggccttttacatccataggcatcacttctctgatattcgttgtgctcttttaatggattaatggtttgcttggttggttcctctagttagactgtaaactccttgagagcagagtctgtattttattaattacccacagtactaggtacatagttgccttcaataaatatatatttaatgaaaaaaaaaaaaaaaa

SEQ ID NO: 2 - DNA, 3061 bp, Homo sapiens
  source 1..3061: mol_type = other DNA
cgcctccccgcccgccccacttctcattcacttggctcgcacggcgcagacagaccgcgcagggagcacacaccgccagtctgtgcgctgagtcggagccagaggccgcggggacaccgggccatgcacgcccccaactgaagctgcatctcaaagccgaagattccagcagcccaggggatttcaaagagctcagactcagaggaacatctgcggagagacccccgaagccctctccagggcagtcctcatccagacgctccgctagtgcagacaggagcgcgcagtggccccggctcgccgcgccatggagcggatccccagcgcgcaaccaccccccgcctgcctgcccaaagcaccgggactggagcacggagacctaccagggatgtaccctgcccacatgtaccaagtgtacaagtcaagacggggaataaagcggagcgaggacagcaaggagacctacaaattgccgcaccggctcatcgagaaaaagagacgtgaccggattaacgagtgcatcgcccagctgaaggatctcctacccgaacatctcaaacttacaactttgggtcacttggaaaaagcagtggttcttgaacttaccttgaagcatgtgaaagcactaacaaacctaattgatcagcagcagcagaaaatcattgccctgcagagtggtttacaagctggtgagctgtcagggagaaatgtcgaaacaggtcaagagatgttctgctcaggtttccagacatgtgcccgggaggtgcttcagtatctggccaagcacgagaacactcgggacctgaagtcttcgcagcttgtcacccacctccaccgggtggtctcggagctgctgcagggtggtacctccaggaagccatcagacccagctcccaaagtgatggacttcaaggaaaaacccagctctccggccaaaggttcggaaggtcctgggaaaaactgcgtgccagtcatccagcggactttcgctcactcgagtggggagcagagcggcagcgacacggacacagacagtggctatggaggagaatcggagaagggcgacttgcgcagtgagcagccgtgcttcaaaagtgaccacggacgcaggttcacgatgggagaaaggatcggcgcaattaagcaagagtccgaagaaccccccacaaaaaagaaccggatgcagctttcggatgatgaaggccatttcactagcagtgacctgatcagctccccgttcctgggcccacacccacaccagcctcctttctgcctgcccttctacctgatcccaccttcagcgactgcctacctgcccatgctggagaagtgctggtatcccacctcagtgccagtgctatacccaggcctcaacgcctctgccgcagccctctctagcttcatgaacccagacaagatctcggctcccttgctcatgccccagagactcccttctcccttgccagctcatccgtccgtcgactcttctgtcttgctccaagctctgaagccaatcccccctttaaacttagaaaccaaagactaaactctctaggggatcctgctgctttgctttccttcctcgctacttcctaaaaagcaacaaaaaagtttttgtgaatgctgcaagattgttgcattgtgtatactgagataatctgaggcatggagagcagattcagggtgtgtgtgtgtgtgtgtgtgtgtgtatgtgcgtgtgcgtgcacatgtgtgcctgcgtgttggtataggactttaaagctccttttggcatagggaagtcacgaaggattgcttgacatcaggagacttgggggggattgtagcagacgtctgggcttttccccacccagagaatagcccccttcgatacacatcagctggattttcaaaagcttcaaagtcttggtctgtgagtcactcttcagtttgggagctgggtctgtggctttgatcagaaggtactttcaaaagagggctttccagggctcagctcccaaccagctgttaggaccccacccttttgcctttattgtcgacgtgactcaccagacgtcggggagagagagcagtcagaccgagctttctgctaacatggggaggtagcaggcactggcatagcacggtagtggtttggggaggtttccgcaggtctgctccccacccctgcctcggaagaataaagagaatgtagttccctactcaggctttcgtagtgattagcttactaaggaactgaaaatgggccccttgtacaagctgagctgccccggagggagggaggagttccctgggcttctggcacctgtttctaggcctaaccattagtacttactgtgcagggaaccaaaccaaggtctgagaaatgcggacaccccgagcgagcaccccaaagtgcacaaagctgagtaaaaagctgcccccttcaaacagaactagactcagttttcaattccatcctaaaactccttttaaccaagcttagcttctcaaaggcctaaccaagccttggcaccgccagatcctttctgtaggctaattcctcttgcccaacggcatatggagtgtccttattgctaaaaaggattccgtctccttcaaagaagttttatttttggtccagagtacttgttttcccgatgtgtccagccagctccgcagcagcttttcaaaatgcactatgcctgattgctgatcgtgttttaactttttcttttcctgtttttattttggtattaagtcgttgcctttatttgtaaagctgttataaatatatattatataaatatattaaaaaggaaaatgtttcagatgtttatttgtataattacttgattcacacagtgagaaaaaatgaatgtattcctgtttttgaagagaagaataattttttttttctctagggagaggtacagtgtttatattttggagccttcctgaaggtgtaaaattgtaaatatttttatctatgagtaaatgttaagtagttgttttaaaatacttaataaaataattcttttcctgtggaagagaaaaaaaaaaaaaaaaaaaaaaaaaaaaa

SEQ ID NO: 3 - DNA, 2718 bp, Homo sapiens
  source 1..2718: mol_type = other DNA
cagggtaacgctgtcttgtggacccgcacttcccacccgagacctctcactgagcccgagccgcgcgcgacatgagccacgggaagggaaccgacatgctcccggagatcgccgccgccgtgggcttcctctccagcctcctgaggacccggggctgcgtgagcgagcagaggcttaaggtcttcagcggggcgctccaggaggcactcacagagcactacaaacaccactggtttcccgaaaagccgtccaagggctccggctaccgctgcattcgcatcaaccacaagatggaccccatcatcagcagggtggccagccagatcggactcagccagccccagctgcaccagctgctgcccagcgagctgaccctgtgggtggacccctatgaggtgtcctaccgcattggggaggacggctccatctgcgtcttgtacgaggaggccccactggccgcctcctgtgggctcctcacctgcaagaaccaagtgctgctgggccggagcagcccctccaagaactacgtgatggcagtctccagctaggcccttccgcccccgccctgggcgccgccgtgctcatgctgccgtgacaacaggccaccacatacctcaacctggggaactgtatttttaaatgaagagctatttatatatattatttttttttaagaaaggaggaaaagaaaccaaaagttttttttaagaaaaaaaatccttcaagggagctgcttggaagtggcctccccaggtgcctttggagagaactgttgcgtgcttgagtctgtgagccagtgtctgcctataggagggggagctgttagggggtagacctagccaaggagaagtgggagacgtttggctagcaccccaggaagatgtgagagggagcaagcaaggttagcaactgtgaacagagaggtcgggatttgccctgggggaggaagagaggccaagttcagagctctctgtctcccccagccagacacctgcatccctggctcctctattactcaggggcattcatgcctggacttaaacaatactatgttatcttttcttttatttttctaatgaggtcctgggcagagagtgaaaaggcctctcctgattcctactgtcctaagctgcttttcttgaaatcatgacttgtttctaattctaccctcaggggcctgtagatgttgctttccagccaggaatctaaagctttgggttttctgaggggggggaggagggaactggaggttattggggttaggatggaagggaactctgcacaaaacctttgctttgctagtgctgctttgtgtgtatgtgtggcaaataatttgggggtgatttgcaatgaaattttgggacccaaagagtatccactggggatgttttttggccaaaactcttccttttggaaccacatgaaagtcttgatgctgctgccatgatccctttgagaggtggctcaaaagctacagggaactccaggtcctttattactgccttcttttcaaaagcacaactctcctctaaccctcccctcccccttcccttctggtcgggtcatagagctaccgtattttctaggacaagagttctcagtcactgtgcaatatgccccctgggtcccaggagggtctggaggaaaactggctatcagaacctcctgatgccctggtgggcttagggaaccatctctcctgctctccttgggatgatggctggctagtcagccttgcatgtattccttggctgaatgggagagtgccccatgttctgcaagactacttggtattcttgtagggccgacactaaataaaagccaaaccttgggcactgttttttctccctggtgctcagagcacctgtgggaaaggttgctgtctgtctcagtacaatccaaatttgtcgtagacttgtgcaatatatactgttgtgggttggagaaaagtggaaagctacactgggaagaaactcccttccttcaatttctcagtgacattgatgaggggtcctcaaaagacctcgagtttcccaaaccgaatcaccttaagaaggacagggctagggcatttggccaggatggccaccctcctgctgttgccccttagtgaggaatcttcaccccacttcctctacccccaggttctcctccccacagccagtcccctttcctggatttctaaactgctcaattttgactcaaaggtgctatttaccaaacactctccctacccattcctgccagctctgcctccttttcaactctccacattttgtattgccttcccagacctgcttccagtctttattgctttaaagttcactttgggcccacagacccaagagctaattttctggtttgtgggttgaaacaaagctgtgaatcactgcaggctgtgttcttgcatcttgtctgcaaacaggtccctgcctttttagaagcagcctcatggtctcatgcttaatcttgtctctcttctcttctttatgatgttcactttaaaaacaacaaaacccctgagctggactgttgagcaggcctgtctctcctattaagtaaaaataaatagtagtagtatgtttgtaagctattctgacagaaaagacaaaggttactaattgtatgatagtgtttttatatggaagaatgtacagcttatggacaaatgtacacctttttgttactttaataaaaatgtagtaggataaaaaaaaa

SEQ ID NO: 4 - DNA, 5666 bp, Homo sapiens
  source 1..5666: mol_type = other DNA
agaagggagacagtggcggtcgcggcggggccgatccgagagttccccttagagaacggagctcacgggcggggaggcctcacctgctagtaggacgcagaaagacagaaggcgaaggagaccccgacttcccgggtcagccccagagccaccccctgccgtagccatcttgcctctctgctgagcggaagcccccgttcggctcctgtctgttagcggcctctctaggctaccactgacaccgtctctgtggcccggagcctaagagaccggaagttcgtgtttccaggcgcttccggaaaccgcgggagagggtcgctgacgtggaggcgtccgaagggcagcagggtgtgtcggggctcggattaagacatcggagtcggagacctgagagatgttaaccaaattcgagaccaagagcgcgcgggtcaaagggctcagctttcaccccaaaagaccttggatcctgactagtttacataatggggtcatccagttatgggactatcggatgtgcactctcattgacaagtttgatgaacatgatggtccagtgcgaggcattgacttccataagcagcagccactgttcgtctctggaggagatgactataagattaaggtttggaattacaagcttcggcgctgtcttttcacattgcttgggcacttagattatattcgcaccacgttttttcatcatgaatatccctggattctgagtgcctccgatgatcagaccatccgagtgtggaactggcaatctagaacctgtgtttgtgtgttaacagggcacaaccattatgtgatgtgtgctcagttccaccccacagaagacttggtagtatcagccagcctggaccagactgtgcgcgtttgggatatttctggtctgaggaaaaaaaacctgtcccctggtgcggtggaatcggatgtgagaggaataactggggttgatctatttggaactacagatgcagtggtgaagcatgtactagagggtcacgatcgtggagtaaactgggctgccttccaccccactatgccccttattgtatctggggcagatgatcgtcaagtgaagatctggcgcatgaatgaatcaaaggcatgggaggttgatacctgccggggccattacaacaatgtatcttgtgccgtcttccaccctcgccaagagttgatcctcagcaattctgaggacaagagtattcgagtctgggatatgtctaagcggactggggttcagactttccgcagagaccatgatcgtttctgggtcctagctgctcaccctaaccttaacctctttgcagcaggccatgatggtggtatgattgtgtttaagctggaacgggaacggccagcctatgctgttcatggcaatatgctacactatgtcaaggaccgattcttacgacagctggatttcaacagctccaaagatgtagctgtgatgcagttgcggagtggttccaagtttccagtattcaatatgtcatacaatccagcagaaaatgcagtcctgctttgtacaagagctagcaatctagagaatagtacctatgacctgtacaccatccctaaagatgctgactcccagaatcctgatgcgcctgaagggaaacgatcctcaggcctgacagccgtttgggtcgctcgaaatcggtttgctgtcctagatcggatgcattcgcttctgatcaagaatctgaagaatgagatcaccaaaaaggtacaggtgcccaactgtgatgagatcttctatgctggcacaggcaatctcctgcttcgagatgcggactctatcacactctttgacgtacagcagaagcggactctggcatctgtgaagatttctaaagtgaaatacgttatctggtcagcagacatgtcacatgtagcactactagccaaacacgaacactcatgccctttgcctcttacagccattgtgatctgtaaccgcaaactggatgctttatgtaacattcatgagaacattcgtgtcaagagtggggcctgggatgagagtggggtatttatctataccacaagcaaccacatcaaatatgctgtcaccactggggaccacgggatcattcgaactctggatttacccatctatgtcacacgggtgaagggcaacaatgtatactgcctagacagggagtgtcgtccccgggtactcaccattgatcccactgagttcaaattcaagctggccctgatcaacagaaaatatgatgaggtactgcacatggtgaggaatgccaaactagttggccagtctattattgcttatctccagaagaagggctatcctgaagtggcactgcattttgtcaaggatgagaaaactcgctttagtctggcactggagtgtggaaacattgagattgctctggaagcagccaaagcactggatgacaagaactgctgggaaaagctgggagaagtggccctgctgcaggggaaccaccagattgtggaaatgtgctatcagcgtaccaaaaactttgacaaactttccttcctgtatcttatcactggcaacttagaaaaacttcgcaagatgatgaagattgctgagatcagaaaggacatgagtggccactatcagaatgccctatacctgggtgatgtgtcagagcgtgtgcggatcctgaagaactgtggacagaagtccctggcctatctcacagctgctacccatggcttagatgaagaagctgagagcctaaaggagacatttgacccagagaaggagacaatcccagacattgaccctaatgccaagctgctccagccacctgcacctatcatgccattggataccaattggcctttattgactgtatccaaaggattttttgaaggcaccattgccagcaaagggaagggaggagcactggctgctgacattgacattgacactgttggtacagagggctggggagaggatgcagagctgcagttggatgaagatgggtttgtggaggctacagaaggtttgggggatgatgctcttggcaagggacaggaagaaggaggtggctgggatgtagaagaagatctggagctccctcctgagctggatatatcccctggggcagctggtggggctgaagatggtttctttgtgcccccaaccaagggaacaagtccaactcagatctggtgtaataactctcagcttccagttgatcacatcctggcaggctctttcgaaacagccatgcggctccttcatgaccaagtaggggtaatccagtttggcccctacaagcaactgttcctacagacatacgcccgaggccgcacaacctatcaggctctgccctgcctaccctccatgtatggctatcctaatcgcaactggaaggatgcagggctgaagaatggtgtaccagctgtgggcctgaagcttaatgacctcatccaacggttgcagctgtgctaccagctcaccacagttggcaaatttgaggaggctgtggaaaaattccgttccatccttctcagtgtgccacttcttgttgtggacaataaacaagagattgcagaggcccagcagctcatcaccatttgccgtgagtacattgtgggtttgtccgtggagacagaaaggaagaagctgcccaaagagactctagaacagcagaagcgcatctgtgagatggcagcctatttcacccactcaaacctgcagcctgtgcacatgatcctggtgctgcgtacagccctcaatctgttcttcaagctcaagaacttcaagacagctgccacctttgctcggcgcctactagaactcgggcccaagcctgaggtggcccaacagacccgaaaaatcctgtctgcctgtgagaagaatcccacagatgcctaccagctcaattatgacatgcacaacccctttgacatttgtgctgcatcatatcggcccatctaccgtggaaagccagtagaaaagtgtccactcagtggggcctgctattcccctgagttcaaaggtcaaatctgcagggtcaccacagtgacagagattggcaaagatgtgattggtttaaggatcagtcctctgcagtttcgctaaggccccctttgtgtgcatgggtcagtcaccatatgttccccccagagaatgtgtctatatcctccttctaacagcaccttccccctgcagctactcttcagatctggctctctgtaccctaaaacctagtatctttttctcttctatggaaaatccgaaggtctaaacttgacttttttgaggtcttctcaacttgactacagttgtgctcataattgtccttgcctttccagcttaattattttaaggaacaaatgaaaactctgggctgggtggagtggctcatacctgtaatcccagcactttgggaggctacggtgggcagatcatctgaggccaggagttcgagacctgcctggccaacatggcaacaccccgtctctaataaaaatataaaaattagcctggcatggtagcatgcgcctatagtcccagctgctcaggaggctgaggcatgagaatcgcttgaacctaggaggtggaggttgcattcaactgagatcataccacttcattccagcctgggtgacagagcaagactctgtctcaaaaaaaaaaaaaaggaaaactctgtgatggacatttgtttagtaaatcccttcagtatttatccctcctttccccacagcagctttctttcctgtcaactagaaaggagcaggatgtaataaatacattttggtgtgactaggccacaccaactcttaatcatctcccattttccttagacatttaaatttcaaggcaggtaccctctgtgtactcagaaatttgaagaagttatttggttttccaaaatgcacactgcgggttattgatttgttctttacaactattgttctcatatttctcacactaaataaatctctatgagagcttcttgacttggccatttatttcttggacactctcatgttcttgttcacccatgcaggcaccccaccaaagtacatatcttccttccagtaataatttttaattacaaaataaacatccactattggaaaaaaaaaaaaaaagctagccgggcatggtggtgggtgcctgtaatcccagctactctggaggctgaggcagaggattgcttgaacccgggaggcggaggttgcagtaagctgagatcgcgccaccgcactccagcctgggcgacagagtgagactccatctcaaaaaaaaagaaagaaaaaaagaagcacatgtttttcatagggtatatatgaggacctaaactgctgtgaaaatgatagaaagcaagtagctcccttattctgtttttgattgcagccttttatcttttgctaattatagcaatatttattgagcacctgccatgtgactgtcactgttctagatattttacatgtaatatacagataaaagaatagtactttatatatattacaatgatacaatgattacattaacaatacaatattttgcttgtcatatgctaagaataattgggtagagtgacattactgtgccttcgattaaaataagtacttttttgcgtgttaaattcatgttttcaataaataataaatgcatatagttgaaaaatcagtaaata

SEQ ID NO: 5 - DNA, 3570 bp, Homo sapiens
  source 1..3570: mol_type = other DNA
agacaggtcaggacatttggtaggggaaggttgaaagacaaaagcagcaggccttgggttctcagccttttaaaaactattattaaatatatatttttaaaatttagtggttagagcttttagtaatgtgcctgtattacatgtagagagtattcgtcaaccaagaggagttttaaaatgtcaaaaccgggaaaacctactctaaaccatggcttggttcctgttgatcttaaaagtgcaaaagagcctctaccacatcaaactgtgatgaagatatttagcattagcatcattgcccaaggcctccctttttgtcgaagacggatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtgaagagcagaaaagatgaatttgtccaattgtgtagacgatatactccctgcccttccccctgcaaaaagaaaaaaagaaaagaaaaagaaaaaaatcccttgttctcagtggtgcaggatgttggcttggggcaacagattgaaaagacctacagactaagaaggaaaagaagaagagatgacaaaccataactgacaagagaggcgtctgctgtctcatcacataaaaggcttcacttttgactgagggcagctttgcaaaatgagactttctaaatcaaaccaggtgcaattatttctttattttcttctccagtggtcattgggcagtgttaatgctgaaacatcattacagattctgctagcctgttcttttaccactgacagctagacacctagaaaggaactgcaataatatcaaaacaagtactggttgactttctaattagagagcatctgcaacaaaaagtcatttttctggagtggaaaagcttaaaaaaattactgtgaattgtttttgtacagttatcatgaaaagcttttttttttttttttttgccaaccattgccaatgtcaatcaatcacagtattagcctctgttaatctatttactgttgcttccatatacattcttcaatgcatatgttgctcaaaggtggcaagttgtcctgggttctgtgagtcctgagatggatttaattcttgatgctggtgctagaagtaggtcttcaaatatgggattgttgtcccaaccctgtactgtactcccagtggccaaacttatttatgctgctaaatgaaagaaagaaaaaagcaaattatttttttttattttttttctgctgtgacgttttagtcccagactgaattccaaatttgctctagtttggttatggaaaaaagactttttgccactgaaacttgagccatctgtgcctctaagaggctgagaatggaagagtttcagataataaagagtgaagtttgcctgcaagtaaagaattgagagtgtgtgcaaagcttattttcttttatctgggcaaaaattaaaacacattccttggaacagagctattacttgcctgttctgtggagaaacttttctttttgagggctgtggtgaatggatgaacgtacatcgtaaaactgacaaaatattttaaaaatatataaaacacaaaattaaaataaagttgctggtcagtcttagtgttttacagtatttgggaaaacaactgttacagttttattgctctgagtaactgacaaagcagaaactattcagtttttgtagtaaaggcgtcacatgcaaacaaacaaaatgaatgaaacagtcaaatggtttgcctcattctccaagagccacaactcaagctgaactgtgaaagtggtttaacactgtatcctaggcgatcttttttcctccttctgtttatttttttgtttgttttatttatagtctgatttaaaacaatcagattcaagttggttaattttagttatgtaacaacctgacatgatggaggaaaacaacctttaaagggattgtgtctatggtttgattcacttagaaattttattttcttataacttaagtgcaataaaatgtgttttttcatgtta

SEQ ID NO: 6 - DNA, 6326 bp, Homo sapiens
  source 1..6326: mol_type = other DNA
ctccttccctccctccctcccccgctgtcctggcccgccctgcccggcccgccctgcgagtcagttcgctggttccctccctccctgggcgcgctcgggccgccgccgcgctccccgccctcgagcctcggtgccggagccgcccgccgccggaggaggaggtggagggagccggaggggcccgccgaggcggcggcggcggcggcaagatggcggacttcctgccgtcgcggtccgtgctgtccgtgtgcttccccggctgcctgctgacgagtggcgaggccgagcagcaacgcaagtccaaggagatcgacaaatgcctgtctcgggaaaagacctatgtgaagcggctggtgaagatcctgctgctgggcgcgggcgagagcggcaagtccaccttcctgaagcagatgcggatcatccacgggcaggacttcgaccagcgcgcgcgcgaggagttccgccccaccatctacagcaacgtgatcaaaggtatgagggtgctggttgatgctcgagagaagcttcatattccctggggagacaactcaaaccaacaacatggagataagatgatgtcgtttgatacccgggcccccatggcagcccaaggaatggtggaaacaagggttttcttacaatatcttcctgctataagagcattatgggcagacagcggcatacagaatgcctatgaccggcgtcgagaatttcaactgggtgaatctgtaaaatatttcctggataacttggataaacttggagaaccagattatattccatcacaacaagatattctgcttgccagaagacccaccaaaggcatccatgaatacgactttgaaataaaaaatgttcctttcaaaatggttgatgtaggtggtcagagatcagaaaggaaacgttggtttgaatgtttcgacagtgtgacatcaatacttttccttgtttcctcaagtgaatttgaccaggtgcttatggaagatcgactgaccaatcgccttacagagtctctgaacatttttgaaacaatcgtcaataaccgggttttcagcaatgtctccataattctgttcttaaacaagacagacttgcttgaggagaaggtgcaaattgtgagcatcaaagactatttcctagaatttgaaggggatccccactgcttaagagacgtccaaaaattcctggtggaatgtttccggaacaaacgccgggaccagcaacagaagcccttataccaccacttcaccactgctatcaacacggagaacatccgccttgttttccgtgacgtgaaggatactattctgcatgacaacctcaagcagcttatgctacagtgatgtacaaaagacttgctgttttaatatctttttgtgtttttgatgttttctgtttgttttgttttttaaaatagcagtttacaaccagaattagaacaatcttaattctacgtttaacttcttgaaaatcttagtactttttctgcggcctttggtttgtggctgaaagctgttgagtgactcatcgccaagatttgctgtaatgcaggctttgatctgtttcaccatggcttctattcaagtccagttaaaacctcccagctgacctcagactaggcatatttcaggctttaaattattctactttccaaactgaattctcctgcagtgccaagtatcaaaggtgtccttaaatacttgtagggatgaggttaggaatattcagttccaaaacaagatatcttctgtccgccttacatatagcagtgacacttgttgcctaactttatggtgacctcctattttgtaagggctgttagaagttctatctaagaaatggcattctgtaggtttatagaaggtttagccttcatattttaattgcttgtatacacaacagctgttttgcttttagatttctgtgtttctgaaggtaatgttcttcctgttttcaagtttacataaggatctttggtctgatgctgatgaagagttcacaggtggtatgggagagcaaaaggcaagctaatgctgtttaccgtgttttggtcaaacgtaacgagtgaaatagaatttgcctttctcatatttaattatcatgtagtttaatgtaccatatgtgaaacattctggccatagcagcaactaaaaactgcaagcaacttggtaacagaactttctaaataaacttaacctgttccagaatgtcatgtatttgacttttaagccctatctcagttggtcagtaaagaccaatccttactgtaggaaatcattgttgtatcatcacaaacatctatcttttgctgtcctgtccagtcccatcaactccacactgtgccatttgtggcatcgttttgtttatttggagtttgctaagggcagtatttttctgtcaagactattcaagaaggcattatttgagattcctgttcattcttggtgtgtctctaacagatacagtatgtatacatttgtataattgttgttgttgaaagtccagcttttttgaggtatattttaaatgttttaaggatgcttctaaggatcagtagtaatttttttagttcgcacctaaagatgattacattgacctcccccgactgcttaccaaattaaaatgtgtccacgaagtagctttgtgatcgcagatacattcatagtgaactcatcagaatggctggtttgcagtactgaaatactatcttctaggctgtatgtagtgctacaattagagaaacagaagtccaaggctggcgacagcttgaaaagtctgacagcttttctacttttcctgaaaattttaagactgtgatatctgtcattttactgtatagctgactgtgtactcaggtattttattggtccttgaaagattggtcgttatggatcacccagcctttccaagtcagtggctgttgttctgtcttgctgtctgatacgagagtggggcttttcagtgaactaaccagggattgttcttgacatacctgacttttctcacatttgaacttccactatcattgtatccatataacttctagcattttcatgccatggtaatccatgagctacacatacgtagcccggcaccgtgatgcaagttcatggtatcgtgcatgttcgtggtatcatggtatcattcatgcgtgtttgaatagttctacatctagtgcttcttgccaaaaagaatacattgtttaaattcacaaaattagcataattgcagtgctaatgaatatcggaatatgtgcacagtaacatttggactattcattggagagtttacccatacatttagcaaattgaatggccaaaacatttgactccagtgagggctcaagttagatccctatagaaagaggacacttcatcttacttaagtcatagttaagatctgtgatacgaaccatagatattgcctgacaaagcagaaatcaccaagtttcccccttttgaattaccaccaagaagtgttgaaacaccaaatagatatcatgttattttgggcatttgcagttttcttccctgctgcatgtaatgtctcagaatcaacattcttttaaaatctagactatattttgaggcaatgaattacttatattcaacttaggcttgttttgacattcagtagaactttaagttcaatctaaaggcttcagtccacatttttttatacgttgtattttaaaaacgtttgaaaggagtcttacacctgtatcatgaaaactgaatccttttgaaataccactatatgaagagagagatgaaatttagtgaacagaattgaaaaggtgctcataatttcactatgcaaacttaccccagtctctaaaaaagtaatttagatttaaagttctttgatgtatttgattttctaaatctttatggttatgatttggaataaaatgtgcctaatcctgtgttacattctgttcttaaatctgaatgccttctcatttaattctgaggaaatatcacacaagtgtcttcattgaccttgaagaaatgtatatacagttgccttataaaacaacataaatttagaccataacttttatagagaaagggttttgtcaaatgttttctgaaaatctgagtaattcaaagcatgcctctgcccctttaatatttttaataacctgcattgttgctgtctgccaaatattaaattgaaatcttcatttcaattttattatctggaaagggcactggattgctctgcaaccaaagaaagcaatatggaatgaaaaaactcattcacttttgtcttattttcttttaaggtgtattggcatgtaatttgcatagagaaggtcctctggttagtctctcaaattgaggctgtttagggaaatccttattcagttggtggcagtggttggtttaaagtagaaggaaataagatcgccttaataccagaaatgattagaagtgctgatttagattcaacaaataccatatgtccttatcattttttgtaagaagaaattggttaagtcctaactttcaatgtgtacccaaatacttgtatttatgcttttgataaaatgtattttcagcattaatacacatccgattatgccttatttatatatgaagaataaagttaccatgttacactgttatgtcctaaaattcaaatcactatttgagaaaccctcaaattggtgctttcattatataatgatacatttagacaaaaccccaaactaagccatttgaaacaagattctctccattgcagtttgtagcaatgttatttctgtgtatgtcatgagaaggctaaatatcagtgttaatttcttgtttgaatccgtgaaatcatgcctgtaaagcccaaacatttgtaacaaactccctaataaatttagagaaagtcactctgttaccatttcatttattttagttttatttgagaaattaagaccagaagttttgctcatgtctttttcacttggaaatctgacttagtacctagttaatttgtctctctcccatcattttaaggcgttcgcttctgttaatcacagatgatctgtccttgtgttgtgctgcttgactaaaagcactgttttgttttgttttgttttgttttttaatgataggatacagggtggcttttcagtctagtaaacatgaggaggggagagattgtgggtgggtgtgggtgtgtccgcatgtcccaaatacagcacatgcccatttttttatggtacgaggttagggcttattaataaatgtatactagatcttgaagtgtaagtctaagtctgaacattttaaacaagttggactttatggctttttcaagttccatcaagtagatactagtcttaggctggtaggtgattaacaacatcatttggagtacagtgtttgtaccactagcattcttatgtctgtacttgaacgtgtagttagcatttaagttatgttcactcttgtagcattcaggtcttaccatctggtttcaaatgcgagagcttatatgaacattgttttgaaagcatgaatgatatggcaatattttatgtcttgataaggagagttttgtgacatatacaaatctgctgttgatgatgaaagtttacaggtggatcagagtatgctggaactcagtgtgcataaaatttcagtcagtgaatatcactgaacgtcatatactacttggtatgtgactttggtttgtgttaagaaagcttgtatataatattttttgccatagtaagtgagaaattgtccttaatcatgcctgtttgatggtactaggaaagaaaggggtagagattaattcttgcacagtataagcaacagtgcaacaaactatgccatttaccttttacctcttacttgaaggcagaatcgcaaaacgtttgaaatggcttttctaaactactctactctggtgagagctcatttaccacaagaagccttataaaaaagtatattttgtaataaccccgtagatactgtacctaacaaaacatgactcgtattagctctataaaatatttgtggcttaggattttttttttacatatatctttttataactttccaggaactaagcacattatctgataattgttgtaaatttttttgtcagtgctttagttcaggatgggcaataaattatttctaaaggaggtctaaaagtggaaagaatggtttgattttataaaatgagttgctagttcattacagctttttacttttgtacatattttgcaaaacatttgcctcttgctattaatatttgctttgtaaaaattactgacatttaataaacatttgtaaacaattcaaaaaaaaaaaaaaaaa

SEQ ID NO: 7 - DNA, 1718 bp, Homo sapiens
  source 1..1718: mol_type = other DNA
gagggtgcataagttctctagtagggtgatgatataaaaagccaccggagcactccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 8 - DNA, 8675 bp, Homo sapiens
  source 1..8675: mol_type = other DNA
ctcgttctcatttgggtgtctgcactgttggaagctactgcactgtgtttccacgcagacagagtacagtgggaaaggctttctccagctgatggatggctgttctctttatttggagtatcttactggttatgtaagcgacctcgagagaaattatcctgacccagatcggaggatgcagtagtgcttcagctgcaggatttcttagtcgttaacaccacagtgctgatgacaaagagcgagttgtaatcctcatcactgccagggctgagccagagcagacagaaaatcgtcatttgctcgttggagacttgttaggttacagctcctgcttcgttctgtggtcttggcttttcttcctatacaatgacgccgctgcaagtgagctcagaaatgcttggtttgagagccatttgcctgtgatttttgaagactaaagaagtatttacactagatactcttccagaacaaaatagaaatttcagcaaatacttactgacctttaaagccttgttgtggatttctgagatttaaagatactttaaaattctcaagaacaaaactttcaaacctctggttgaaagaaatatacccagttcagtcactgcagtagaattccttgtagataagcaactggattttttaactgaagatagtgcctttcagccctaccaggacgacatagacagcctaaacccagttctcagggacaacccgcagcttcatgaggaagtgaaagtctgggtaaaggaacaaaaggttcaggagatttttatgcaaggtccttattccttaaatggatacagagtgagagtatatagacaagactctgccacccagtggtttactggcataattactcatcatgatctcttcacccgcaccatgatcgttatgaatgatcaggtactagaaccacagaatgtcgatccttctatggttcaaatgacctttctagatgatgttgttcactctttgttaaaaggtgaaaatattggcattacatcacgacgcaggtctcgtgccaatcaaaacgtcaacgctgttcacagccattatacacgtgcccaagcaaatagtcccagaccagcaatgaactcccaagctgctgtaccaaaacagaatacacaccagcaacagcaacaaagaagtatccgtccaaataagaggaagggctcagatagcagtataccagatgaagagaagatgaaggaggaaaaatatgattatatatcacgaggagaaaatcctaaaggtaaaaacaaacacttgatgaataaaagaaggaaacctgaggaggatgaaaagaaactaaatatgaaaagacttcgaactgacaatgtttcagacttttctgagagcagtgactcagaaaattcaaataagagaataatagataattcctcagaacagaagccagagaatgaattgaaaaataaaaatacttcaaaaataaatggagaagaaggaaaaccccataataatgagaaggcaggagaagagaccctaaaaaatagccagcctccctgggatcaaatacaggaagataaaaaacatgaagaagcagagaagcggaagtctgttgacactcagcttcaagaagatatgattattcattcgtcagaacagtccacagtttctgatcataattctaatgatttacttcctcaggaatgcaatatggataaaacacataccatggaattgctaccaaaggagaagtttgtatccagaccacccacaccaaaatgtgttattgatattacaaatgacactaatttagaaaaggtggctcaggaaaactcaagtacctttggccttcagacacttcagaaaatggatcctaatgttagtgattcaaaacactctattgcaaatgcaaaattcttggaaacagcaaaaaaagattctgaccagagctgggtcagtgatgtagttaaagtggatctaacccaatcaagtgttacaaatgcttcttcaggaaatgatcacttgaacatggaaaaagagaagtatgtctcttacatttctcctttaagtgcagtttctgtcatggaagataagctgcataagcgaagtccacctccagagactataaaatctaaacttaatacttcagtagatactcacaagataaaatccagcccatcacctgaagttgttaaacccaaaataactcattctcctgattctgtaaagtctaaggccacttatgtgaacagccaagctactggtgaaagaagattggcaaataagatagaacatgagctatcaagatgcagttttcatccaattcctactcgaagcagtacattagaaactacaaagagtcctcttatcattgataaaaatgagcattttacagtttacagagatcctgcacttattgggtcagaaacaggagctaatcatatttcacctttcctaagccagcatccttttcctcttcactcctcatctcatagaacctgtttaaatccaggtacccatcatcctgccttaactcctgcaccccatttactagccggatcatctagtcaaactccattacctaccattaacactcatcctctgactagtggtccacaccatgctgttcatcaccctcatttacttcccactgtgttacctggagtgcctactgcctccttacttggtggccacccacgactagagagtgctcatgccagcagcttgagccacttagcgctagcacaccagcaacaacaacagttgttacagcaccagtcacctcatcttcttggacaagcccatccttctgcttcatataatcagcttggactttatccaattatttggcagtatccaaatggaacacatgcatactcaggacttggtttgccttcttctaagtgggttcacccagaaaatgcagttaatgctgaagcttcattaaggaggaattctcccagtccttggctacatcagcccacccctgtgacctcagcagatggtattggattacttagtcacattcctgtcagaccttccagtgcagagcctcatcggcctcttaaaattacagcccattccagtccaccattgacaaaaactttagtagatcatcataaggaagaattagaaagaaaagcttttatggaaccattacggtctgttgcatccacatcagccaaaaatgacctggatctaaataggtcacagactggaaaagattgtcacttacataggcattttgtggatccagtattaaatcagttacagaggccaccccaggagactggagagaggttaaacaaatacaaagaggaacaccgtcgaattcttcaagaaagtattgatgttgctccctttacaactaaaatcaagggacttgagggtgagagagagaattattccagagtggcatcatcatcttccagtcctaaaagccatatcatcaaacaagatatggatgtagaacgctcagtatcagatctttataaaatgaagcactcagtgcctcagagtttaccccaaagtaactatttcactacattgtctaatagtgtggtcaatgaaccaccaagatcatacccatccaaagaagtttcaaatatttacggtgataaacagagtaatgcccttgcagcggcagcagctaatcctcaaactctgacttcatttataacatctctttcaaagcctccacctttgattaaacaccaaccagaaagtgaaggtttagtaggcaagataccagaacatcttccacatcagattgcatctcactcagtaacaaccttcagaaatgattgtaggagtcctacccatttgacagtttcttctacaaatacactccgcagtatgcctgcattacatagagcaccagtatttcacccaccaatccatcacagcctggaaagaaaggaaggcagctatagtagtctttcccctccaactttaactccggtgatgccagtaaatgctggtggtaaagttcaagaatcacagaagcctccaactctaatacccgaaccaaaagactcccaggcaaattttaagagttcttcagaacagagtttgacggagatgtggagacctaataataacctcagcaaagagaaaactgaatggcatgtggagaaaagcagcggaaagttacaggctgctatggcatctgtcattgtgcgtccatcttctagtacaaaaactgatagtatgccagcaatgcagttagcttctaaagatcgagttagtgaaagatcttcagctggggcacataaaacagattgcctcaaactagcagaagccggagaaactggaagaatcattttgccaaatgtgaattcagacagtgttcacacaaaatctgaaaaaaactttcaggctgtctcacagggcagtgttcccagttcagtcatgtctgctgtaaatacgatgtgtaataccaaaacggatgtaatcacatctgctgccgatactaccagtgtttccagctggggtggttcagaagtaatttcctctttatcaaataccattttggcctctacatcatcagaatgtgtatcttcaaaaagtgtcagtcagccagtggctcaaaaacaagaatgcaaggtcagcaccacagcaccagttacattagccagtagtaagacaggaagtgttgttcaacccagttctgggttctcaggcacaactgattttatccatttaaaaaagcacaaggcagcattggctgcagctcagtataaaagtagtaatgccagtgagactgaacctaatgctataaaaaatcagacactttcagcctcccttcctctggatagcactgtaatctgtagtacaattaacaaagcaaactctgtaggaaatgggcaagcttcccagacaagtcaaccaaactaccatactaaactgaaaaaggcctggctcaccagacactcagaagaagataaaaatactaataaaatggaaaattcagggaattctgtatcagaaattattaagccatgttctgtcaacttaatagcctctacatctagtgatatacaaaatagtgtagatagtaagatcatagttgataaatatgtaaaagatgataaagtcaacaggagaaaagccaaaagaacttatgaatctggctctgaaagtggagactcagatgaaagtgaaagcaagtcagagcaaaggactaaaagacaacctaagccaacttacaaaaagaagcaaaatgatttgcaaaagagaaaaggtgaaatagaagaagatttgaaacccaatggagttctcagcaggagtgccaaagaaagaagtaaactgaagttgcaaagcaacagtaatactggcattcctcgttcagtattgaaagattggcgtaaagtcaagaagctgaagcaaactggggaatcctttttacaggatgactcctgctgtgagatagggcctaatttacaaaagtgtcgagaatgtagacttattcgcagtaaaaaaggagaagaaccagctcactcaccagtattttgtagattttactactttagacggttgtcatttagtaaaaacggagtagttagaatagatggtttctcttctcctgaccaatatgatgatgaagctatgagtttgtggacacatgaaaattttgaagatgatgaactagatatagagacttctaaatatatcttggatataataggtgataagttctgtcaattagtaacatctgaaaaaacagctttgtcctgggtgaaaaaggatgccaaaattgcctggaaaagagcagtgagaggagtccgggagatgtgtgatgcatgtgaagcaacattgtttaacattcactgggtctgccaaaaatgtggatttgtggtctgcttagattgttacaaggcaaaggaaaggaagagttctagagataaagaactatatgcttggatgaagtgtgtgaagggacagcctcatgatcacaaacatttaatgccaacccaaattatacctggttctgttttgacagatcttctagatgccatgcacactcttagggaaaaatatggtattaaatcccattgtcattgtactaacaaacagaatttacaagttggaaattttcctacaatgaatggtgtatctcaagttttacagaatgttcttaatcacagtaataaaatttctctgtgcatgcctgagtctcagcagcaaaatactcctccgaagtctgagaaaaatggtggcagcagcccagagagtgatgtaggcacagataacaagttaactcctccagaatcccagtcaccactgcactggttagcagatcttgcagagcaaaaagccagagaggaaaaaaaagaaaacaaagaacttacccttgaaaaccaaattaaagaagaaagagaacaagacaactctgaatctccaaatggcagaacatcacctcttgtgtcccagaataatgaacaaggctcaaccttacgggatttgctgactacaacagctggaaagctacgtgtggggtctacagatgctggcattgcctttgccccagtatattcaatgggagccccaagtagcaaaagtggacggactatgcctaacattcttgatgacataattgcttcagttgttgaaaacaaaattccaccaagtaaaacctccaagataaatgtaaaaccagagcttaaagaagagcctgaagaaagcataatatctgcagtggatgaaaataataaattatacagtgatataccacattcttggatctgtgagaagcatattttatggcttaaggattataagaatagcagtaattggaagcttttcaaagaatgttggaaacaaggacagcctgcagtggtttctggtgtgcataagaaaatgaacattagcctatggaaggcggaatcaattagtcttgattttggagaccaccaagctgatctcctgaactgcaaagatagcatcatttcaaatgccaatgttaaggaattctgggatggttttgaagaagtttcaaaacggcagaaaaacaagagtggagaaacagttgttttaaaattgaaagactggccttcaggagaagacttcaagactatgatgccagcaagatacgaagatcttttaaaaagtctgccattgccagaatattgtaatccagaaggaaaattcaatttggcctctcatttgccaggattttttgtacgtcctgatctaggacccaggttgtgcagtgcctatggtgtagttgctgctaaagatcatgatataggaacaacaaatctccatattgaagtttctgatgttgtaaatatactagtttatgttggcatagcaaaaggaaatggcattctctcaaaagcaggaattctcaagaaatttgaggaagaagatttggatgacattttaaggaaaagattgaaggactcaagtgaaatacctggtgctctgtggcatatttatgctgggaaagatgttgacaagataagggaatttcttcaaaagatttcaaaagaacaaggccttgaagttctaccagaacatgatccaatacgtgaccaaagttggtatgtgaacaaaaagctccgtcaaaggctgcttgaagaatatggagtcagaacctgtactcttattcagttccttggtgatgctattgttttgccagcgggagcacttcatcaggttcagaattttcacagctgtattcaggtaactgaagattttgtgtctccagaacatcttgtagagtcatttcatttaacacaggaactgagacttttgaaggaagaaatcaattatgatgataaactacaggttaaaaatattttgtatcatgcagtcaaagaaatggtgagagccttgaagatacacgaggatgaagtagaggatatggaagaaaattaagtgtgatccagtttgatatttttaggttgttgaactgggattacttaaccttgaatgatgatatgtatgcacactgactttaagcttcataaaaccatcagtgccaagaaattctctttgtagtaattacttgttactgacaccacagcagtatagcatatgtcacagctcctgtgattcaatgttataaaacaagcagaattttaaaagcagcactatatagctgttttgtattatagtgtatatgatgtttgtgaaaatgccagatttaaaatgatgtatttatttttggtaaaaaataaaaaattctatgctatattgttgatcaagtgtaaatgtgaccttgtacagtttactaaaattactgatatttttcactacattgagacagttactgtgagaataggacacaaacaccagctattgcctgcatctgggaaattgctgaatcgcacagcagtcatgtcataatcagaaaattactgccaaataattgtaaaatttgtaaagtataaagtatataaagtagatactaaatacagacacttcaatattttgttgaagctattgactgtacaattaaacattttcaaaaggtgtaatttatttaaaattgtctcattttggtaaaatttatgtgaacttttaaagctaaatattaaacttaatatgctatgtaaatatatacatatatacatttaatgatgtatttttttaaaacattggcttgcttttgttaaagtgcaagtgttacatatggctttgtacattaaagttgaaaggggttttacattttccattaaaaggactttatcaaaaattga

SEQ ID NO: 9 - DNA, 4203 bp, Homo sapiens
  source 1..4203: mol_type = other DNA
tgacaacatggcggcgcccatggtccgtggcccggcagtgctcgcctaaaggtggagaacgaggagtagaggagccgcaggccagagcctgtgagcagatccagacctacagataaaaaacattatttaatctatctgggatttactccggcttatgatttgagggccttctcaccttctgaagaatggcttctgtttggcagagattgggtttttatgcctctcttctgaaaagacagctaaatggtgggccagatgtcatcaagtgggaaaggagagtaattcccggatgtaccagaagcatctacagtgccacgggaaagtggacaaaagagtatacattgcagacaagaaaggatgttgagaaatggtggcatcaacgaataaaagaacaggcctccaaaatttcagaagctgataaatcgaagccaaaattttacgtgctttccatgttcccttatccttctggtaagctgcacatgggccatgtgcgtgtctacaccatcagcgacaccatagcacggttccagaagatgagagggatgcaggtcatcaaccccatgggatgggatgcttttggattgcctgctgaaaatgccgcagtcgagaggaatctacatccacaaagttggacacaaagtaatattaaacacatgaggaaacagcttgatcgtctgggcctgtgtttcagctgggatagggaaataactacgtgtttgccagattactacaagtggactcagtatctctttattaaactgtatgaggctgggctggcctatcaaaaggaggccctggttaactgggacccagtggatcaaacagtgcttgccaatgagcaggtggatgaacatggctgttcatggcgttctggagcaaaggtggaacagaagtacctcagacaatggtttattaagacaaccgcttatgcaaaggccatgcaggacgcgttggcagaccttccagaatggtatggaataaaaggcatgcaagcccactggattggggactgtgtgggctgccacctggacttcacattaaaggttcatgggcaagccacgggcgaaaagctgactgcctatacggccacccctgaagccatttatggcacctcccacgtggccatctcgcccagccacagactcctacatgggcacagctctctgaaggaagccttgaggatggcccttgtccctggcaaagattgcctcacgcctgtaatggctgtgaacatgcttacccagcaggaggtccctgtcgttattttggccaaagctgacttggaaggctctctggattcaaaaataggaattcccagtactagctcagaggacaccatcttagcccaaaccctgggcctggcctactctgaagtcattgaaactttgccagatggcacagagagactgagcagctctgctgagttcacaggtatgacccggcaggatgcttttctagccctgactcagaaagcccgggggaagagagtgggtggagacgtgacaagtgataaactgaaagactggctgatttcacggcagcggtactggggcacaccaatccccattgtccactgcccagtctgtggccccacacctgtgcccctggaggacttgcctgtgaccctgcccaacatcgcgtctttcactggcaagggaggccccccactggccatggcttcagagtgggtgaactgctcctgcccaaggtgcaagggagcagccaagagagagacagacacgatggatacctttgttgattctgcttggtactacttcagatacactgaccctcataatccacacagcccttttaacacagcagtggccgattactggatgcctgtggatttgtacattggagggaaagaacatgccgtcatgcacttgttctatgcaagattctttagtcatttttgccatgatcaaaaaatggttaaacatagggagccttttcataagctgctggcccaaggccttatcaaggggcagacattccgcctaccatctggacagtatctacagagagaggaagtggatctcacaggttccgttcctgttcatgcaaaaacgaaagagaagttagaggtgacgtgggagaagatgagtaagtccaaacacaacggggtggacccagaggaagttgtggagcagtatgggatcgacacgattcggctctacatcctttttgctgcccctcctgagaaggatatcttgtgggatgtgaaaactgatgctctccctggggtgctgagatggcaacaacgactgtggaccttgacaactcggtttattgaggccagggcttctgggaagtctccccagcctcagctgctgagtaacaaggagaaagctgaggccaggaagctctgggagtacaagaactccgtcatctctcaggtgaccacccatttcacagaggacttctcactgaattctgcaatttctcagctgatgggactcagcaatgccctctcgcaagcctctcagagcgtcattctccacagccccgagtttgaggatgctttgtgtgccctgatggtaatggctgctccactggcccctcatgtaacctcagagatctgggcaggcctggcgctggtgccgaggaagctctgtgcccactacacttgggatgccagtgtgctgctccaggcatggcctgctgtggacccggagttcctgcagcagcctgaggttgtccagatggcagttctgatcaacaataaagcttgtggcaaaattcctgtgccccaacaagttgcccgggaccaggacaaagtccacgaatttgttcttcaaagcgagctgggtgtcaggcttttgcaaggacgaagcatcaagaagtccttcctttccccgagaactgccctcatcaacttcctggtgcaagattgacagccaggaggctgcagctaccacgagggcctctgaggaacctccttccaggcctgggatgagggggcgatgtctgctggcccaggggaagggaaaagacaaatgtcttgactgttgacctcggtcctgtggcagactgcagtcaacagtgtgcctctgtagtgtggcctggtgctggggtgaaggtgagctgggcaaaggagaaatatgagctactgaggagggggttggacatcctgcccctcaccccccacccacactgcaggtagaggaggccatctgatcccatgggaagccatcagagacactgctggtgggagcaggaaggagcagtgcccctcgagcagccaggaagcctgcggatctgggaaatggctctgccttaggcacttctcgggaatttgaggccagcctgaggaactgcaggactcaggtgcaatgtgccagccacttggaactgctaactgagcctccagatggtagtgaatggtctctttgccttcaggctggatgaggaagtcatttaggaaatgttcaaataaccaatatgtggaaatggacacagggatcttctgaagttgctttgaatcaaaaggcaggcagtgctggttcctctgcctgtgtccccaccactccccagctctgtcatgcaggcctgtcctccccaaccccagctggatgtgcctcccaggcctgctgtggttctgacacacaggatcccaggcaaggcaccacttcctcacatgaatgaggagcagcaagtcataaccactcccttgggtatacaatttgctgtgtagtgaagtggaaccaggctcaggctgctggtcccaacctcagagccccaccgcagcccagtagggatgcagcacgccccagagggctcatgtgggccccagatggcaatgccaccattgttgatgtgactccagagccagttattaggaagagcaagctcaccacagaggagtggaactgaggccccccagatgttgcctccggtgtccaagccacagcggtctggctgttgggaagatggccaggaatggactcataccattggcacattaggctaatcctggttttatgtgaagtcagcaattaagtgttcccactagaactgacctaagccactgattaatatttaatgagggaaggtaggggagaatctagccattttataatgccagaaatctatatatgttatctgatgccatttttctgaagtagcctcacatgtggtccccctgcagttcagcagttaacagatgacttttttagtgtaataaaatgtttatcatctatg

SEQ ID NO: 10 - DNA, 8708 bp, Homo sapiens
  source 1..8708: mol_type = other DNA
gtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggtattttaaaagttccgggggttttgtgaggtgtttgatgacccgtttaaaatatgatttccatgtttcttttgtctaaagtttgcagctcaaatctttccacacgctagtaatttaagtatttctgcatgtgtagtttgcattcaagttccataagctgttaagaaaaatctagaaaagtaaaactagaacctatttttaaccgaagaactactttttgcctccctcacaaaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaa

SEQ ID NO: 11 - DNA, 5266 bp, Homo sapiens
  source 1..5266: mol_type = other DNA
cttgccggcttccttgcaaagcccggtgcaagggcctctttcaaaatgaacccactggtgtgcctagcagtcggtagaagaagcgggagggcgtccggtctgcacgcccgccgcgaggttacaatgctgaacgcatgagatggaagataccaacgggaggccgaggggatccacggcgcccgcgcgggctccggcttcctcctgctctcggcgccgctgggcgaccgcccatgacccgctcttgcgggctctgtccggttgacaggcgaccctgtggcccggggaagcgcgggagggcgccggcggaaagttgaagagcgtttttctcgccgccgcgtgcattaggagctcgacgagtccgccctgggcttcctggtggggctgggcgggcgggggaggggccgcgcagcagcagcggaagccagacctcggcgataagaggctgcacagcgacatgcaacagtcttttcactgcagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagagcagtcactcaggaaatgttgtttaaggggaaccttctggatccttttcatggcaccatggcaagaagaagctgtatcttatctatggaagataaagcatggagttggctaatggatgctgataggaccatctagttgcaggaaaacaagctcagggctcccactgattctacattatgggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgactcagtcggctgtcaaatcactgaagcgacccctcgaggcaacctttgacctgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccaggctcaatattgtgcatgacacccgctacaagtgttgttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtagaattttcatcactaaacaatcatgctaaagaggaaaggacagtgtgcttggttagagtaaaggacgaggtcattagccatattgtatatatcgtcaagcaacacacacaaaagttcctcagccacaagacatccacatattgcatgttaaccagaagaaaagacaacattttccggaaatccactgcacactgttgcctatacactttgtacatttaattgatatttgtgctgaggtgatattcctgtctaaaagaacaacattgtctttcttttctagcacagagttatgcattcaaagatgcatacctagttagtttcctatatattcatgccatcttgaaaagacagactatggtgtaaccatgattctattatgtattggtacgtctgtagaccaagatataattttttaaaaataagtttatttctttcaaggtttacaaataacaaaggtgcaccttgtatttaaaattgccattatagatgagagcgtgcatgcacagtcatttttgtttaagagtaatatttttaatgtaatagattgtaagacgtggtgagggagggatctgacagagatgaatgtgccaagcaaaaccacaactgtgtatattttaaagcacatcatggctttaagtaccatgttgttaaggattctcatgaagtgccatagactgtacatcaaattagagtattatttcttcagtgttattgttttcagagccacattttgttgcatatttgctagtactaatcagtcaaagggcaccattctttttttttttttttgaaaccaaagctgtctcagaaatggccaatttaactttacagtaacaatagacagcacaacacaaactctctcaatacagataaactcacacatactggagatatatatataatagatatatataaaattattttaatgcattgtagtgtaatatttatgcatactatactgtataacatgttattcaaaagggattgccatttctgagacacagtaacaaaaaaatgaggaaattattttgcttctatttatagcctctgtcaaaagtcaaaagactataaatgctttgcaaaaatggtttcacgtttgcttaaatgcttcatcacagtcacattcaaaatagtgactctaaacaaagaagaaagcagcactgtcatcagatgcatgataaaccaaaatatgaaaatgggaaatgtttaattaacctagtaattgggtgggttaagtacatgggtgaattttatatgtgatttttgttttgttttgttttgttcagattaactgcttatagccttagaaagccttttacaaaattaaaaaaaaaatagatgtgcattcagtttttaagaatggaatcatccaaaggaattcctttttttgaggtttggatgttgcagctagtaaaggatatttttgctctgttcagcagttctaaaaattgctgaagtaggggccaggtcactggtagttatagtatggaatgggagaagtgaaagttcagttatagaactttccatacttccaagtttactgcaagtttttatgcttgagagagatgctttctaatataagactgatgtgttgattttactgattgtactgtacatctattaaagccttagattattacattacgggttggaacccataccaatgtaatttcaatcgtgttaagaaagtaatggtgacttcacatgttattgtagttagttacattatagaatattacttatttttcttgttaaaatgtagtttttcatttcctacatttattagattttcattttctattaacaattgaataccatttcagtttatagacttgttttattagattttaccaatgaatttttcaaaatacaaaaaaaagtagtttttccttcataacatactcagttttgaattacatgtagtgtcacatgaatattcgtattgttaactaaatgatttatattttactgatttaatattacagtgtaagaatgtcagtcattgttagttcttgtctagttttcattaaaagaacaaagatcttttatatggatatcttataaatatataatcattgctaagtaagaagttaagttgttgctatcgcaacaatcctggcagacaattgagtaatattttgatgatttattttgtttgtaattagttattataagaagatctagatcctagatattagaataaaatttattttctactgtatccatttcaaatgttaaaatattgtttaatatttttgaaatccctgagtatcaggccttgttataaataagctgcataatcaataaatagaacaagggactttttgttgataatccaaatactcaaagtttacgtaatgaaaattatagcgtgtgtgcaaactcttgagggttgattatgctgcaatttagcatgttggaacgtctagggagaaggttgactttttgcacttctgtatatagtcaaaagagagaaacctgtataatagtaagatcttattttgaataaaaacgtctataattacaaggagttttgttaaggctaatacaatgacagactgagcaaaattgcttgcaaaagtggcacagagttagcactccataccccttcaaacatgttgctttgctttcttgtggacagcttgtagtttgccaggattttttcagctggaaagatacgccatcctttcaaaccctcatgactgacaaaaactccatggggccaaatctgcctgaagatcattaccaaaaatagcaggtacttctaccattaaggtgaaatcatggatcagatattccttacatttttcaaaactactgcatgtttaaaacttcaacaaaaaaagagagaaagaactatactaagaacatatattattcagatcagtttctgccaatttcagtggtttattgttcacaaaaaaatcttcaaaacaagtattgactttcacaaaatttaaatcataaacaggcaaaccaaacagcacactgtagctatagttgttatgtgattgttttttaattgctgtaggatcctgttctttcagcaggtgaaaaataaaacgcagttcaaatttcatggttttaattttcaactcagaagcactcaaaaatgcaaaatgtgataatgggcacttgtttaaaagaattagtgtatccagccttcactccagctggttaaaaatgttgcacttatcagcaaccctaccactttcatctgctgaaaggacaaatgtgcttggttttactattatgtaatcacaacttactttctgcttgtagttgcttaaaattatgtattttgtcttgggctgcaatttgttttatgcttattttattattactgcagtagttgactttgctgtatggaaaaataaagtgaaattgccctaataaaacttctctttcttaagtaaaaaaaaaaaaaaaaaaaaa

SEQ ID NO: 12 - DNA, 3648 bp, Homo sapiens
  source 1..3648: mol_type = other DNA
gcgcaaccctccggaagctgccgcccctttccccttttatgggaatactttttttaaaaaaaaagagttcgctggcgccaccccgtaggactggccgccctaaaaccgtgataaaggagctgctcgccacttctcacttccgcttccttccagtaaggagtcggggtcttccccagttttctcagccaggcggcggcggcgactggcaatgtttggcctcaaaagaaacgcggtaatcggactcaacctctactgtgggggggccggcttgggggccggcagcggcggcgccacccgcccgggagggcgacttttggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatagccttactgtaagtgcaatagttgacttttaaccaaccaccaccaccaccaaaaccagtttatgcagttggactccaagctgtaacttcctagagttgcaccctagcaacctagccagaaaagcaagtggcaagaggattatggctaacaagaataaatacatgggaagagtgctccccattgattgaagagtcactgtctgaaagaagcaaagttcagtttcagcaacaaacaaactttgtttgggaagctatggaggaggacttttagatttagtgaagatggtagggtggaaagacttaatttccttgttgagaacaggaaagtggccagtagccaggcaagtcatagaattgattacccgccgaattcattaatttactgtagtgttaagagaagcactaagaatgccagtgacctgtgtaaaagttacaagtaatagaactatgactgtaagcctcagtactgtacaagggaagcttttcctctctctaattagctttcccagtatacttcttagaaagtccaagtgttcaggacttttatacctgttatactttggcttggtttccatgattcttactttattagcctagtttatcaccaataatacttgacggaaggctcagtaattagttatgaatatggatatcctcaattcttaagacagcttgtaaatgtatttgtaaaaattgtatatatttttacagaaagtctatttctttgaaacgaaggaagtatcgaatttacattagtttttttcatacccttttgaactttgcaacttccgtaattaggaacctgtttcttacagcttttctatgctaaactttgttctgttcagttctagagtgtatacagaacgaattgatgtgtaactgtatgcagactggttgtagtggaacaaatctgataactatgcaggtttaaattttcttatctgattttggtaagtattccttagataggtttttctttgaaaacctgggattgagaggttgatgaatggaaattctttcacttcattatatgcaagttttcaataattaggtctaagtggagttttaaggttactgatgacttacaaataatgggctctgattgggcaatactcatttgagttccttccatttgacctaatttaactggtgaaatttaaagtgaattcatgggctcatctttaaagcttttactaaaagattttcagctgaatggaactcattagctgtgtgcatataaaaagatcacatcaggtggatggagagacatttgatcccttgtttgcttaataaattataaaatgatggcttggaaaagcaggctagtctaaccatggtgctattattaggcttgcttgttacacacacaggtctaagcctagtatgtcaataaagcaaatacttactgttttgtttctattaatgattcccaaaccttgttgcaagtttttgcattggcatctttggatttcagtcttgatgtttgttctatcagacttaaccttttatttcctgtccttccttgaaattgctgattgttctgctccctctacagatatttatatcaattcctacagctttcccctgccatccctgaactctttctagcccttttagattttggcactgtgaaacccctgctggaaacctgagtgaccctccctccccaccaagagtccacagacctttcatctttcacgaacttgatcctgttagcaggtggtaataccatgggtgctgtgacactaacagtcattgagaggtgggaggaagtcccttttccttggactggtatcttttcaactattgttttatcctgtctttgggggcaatgtgtcaaaagtcccctcaggaattttcagaggaaagaacattttatgaggctttctctaaagtttcctttgtataggagtatgctcacttaaatttacagaaagaggtgagctgtgttaaacctcagagtttaaaagctactgataaactgaagaaagtgtctatattggaactagggtcatttgaaagcttcagtctcggaacatgacctttagtctgtggactccatttaaaaataggtatgaataagatgactaagaatgtaatggggaagaactgccctgcctgcccatctcagagccataaggtcatctttgctagagctatttttacctatgtatttatcgttcttgatcataagccgcttatttatatcatgtatctctaaggacctaaaagcactttatgtagtttttaattaatcttaagatctggttacggtaactaaaaaagcctgtctgccaaatccagtggaaacaagtgcatagatgtgaattggtttttaggggccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttctgtttttcctgagaaaaaaaaataaatcttttattcaaatacagggaaaaaaaaaaaaaaaaaa

SEQ ID NO: 13 - DNA, 3801 bp, Homo sapiens
  source 1..3801: mol_type = other DNA
ctcctcttgccacgaggtcagacggcgagttcttagagaaaaaggctgcttagctgctgcttatcatgtaacctcaaaaggaaactgatcgtctttctcatgctgtcacgtacttgggttattatcgctgattacagctggaaacaattgatttgctcttacgtatttgtgtgacttgactcttcaaacacaaaggttaacaggaagatctcgagggccctggctgaacttcaccttttggctttcttggcctgatgctgaactctcgaggttgagccccatatgggggttggcaggcagcagagaggcccctttcaaggtgttcgggtaaagaactcagtgaaggaactcctgttgcacatccgaagtcataaacagaaggcttctggccaagctgtggatgattttaagacacaaggtgtgaacatagaacagttcagagaattgaagaacacagtatcatacagtgggaaaaggaaagggcccgattcgttgtctgatggacctgcttgcaaaaggccagctctgttgcattcccaatttttgacaccacctcaaacaccaacgcccggggagagcatggaagatgttcatctcaatgaacccaaacaggagagcagtgctgatctgcttcagaacattatcaacattaagaatgaatgcagccccgtttccctgaacacagttcaagttagctggctgaaccccgtggtggtccctcagagctcccccgcagagcagtgtcaggacttccatggagggcaggtcttttctccacctcagaaatgccaaccattccaagtcaggggctcccaacaaatgatagaccaggcttccctgtaccagtattctccacagaaccagcatgtagagcagcagccacactacacccacaaaccaactctggaatacagtccttttcccatacctccccagtcccccgcttatgaaccaaacctctttgatggtccagaatcacagttttgcccaaaccaaagcttagtttcccttcttggtgatcaaagggaatctgagaatattgctaatcccatgcagacttcctccagtgttcagcagcaaaatgatgctcacttgcacagcttcagcatgatgcccagcagcgcctgtgaggccatggtggggcacgagatggcctctgactcttcaaacacttcactgccattctcaaacatgggaaatccaatgaacaccacacagttagggaaatcactttttcagtggcaggtggagcaggaagaaagcaaattggcaaatatttcccaagaccagtttctttcaaaggatgcagatggtgacacgttccttcatattgctgttgcccaagggagaagggcactttcctatgttcttgcaagaaagatgaatgcacttcacatgctggatattaaagagcacaatggacagagtgcctttcaggtggcagtggctgccaatcagcatctcattgtgcaggatctggtgaacatcggggcacaggtgaacaccacagactgctggggaagaacacctctgcatgtgtgtgctgagaagggccactcccaggtgcttcaggcgattcagaagggagcagtgggaagtaatcagtttgtggatcttgaggcaactaactatgatggcctgactccccttcactgtgcagtcatagcccacaatgctgtggtccatgaactccagagaaatcaacagcctcattcacctgaagttcaggagcttttactgaagaataagagtctggttgataccattaagtgcctaattcaaatgggagcagcggtggaagcgaaggatcgcaaaagtggccgcacagccctgcatttggcagctgaagaagcaaatctggaactcattcgcctctttttggagctgcccagttgcctgtcttttgtgaatgcaaaggcttacaatggcaacactgccctccatgttgctgccagcttgcagtatcggttgacacaattagatgctgtccgcctgttgatgaggaagggagcagacccaagtactcggaacttggagaacgaacagccagtgcatttggttcccgatggccctgtgggagaacagatccgacgtatcctgaagggaaagtccattcagcagagagctccaccgtattagctccattagcttggagcctggctagcaacactcactgtcagttaggcagtcctgatgtatctgtacatagaccatttgccttatattggcaaatgtaagttgtttctatgaaacaaacatatttagttcactattatatagtgggttatattaaaagaaaagaagaaaaatatctaatttctcttggcagatttgcatatttcatacccaggtatctgggatctagacatctgaatttgatctcaatggtaacattgccttcaattaacagtagcttttgagtaggaaaggactttgatttgtggcacaaaacattattaatatagctattgacagtttcaaagcaggtaaattgtaaatgtttctttaagaaaaagcatgtgaaaggaaaaaggtaaatacagcattgaggcttcatttggccttagtccctgggagttactggcgttggacaggcttcagtcattggactagatgaaaggtgtccatggttagaatttgatctttgcaaactgtatataattgttatttttgtccttaaaaatattgtacatacttggttgttaacatggtcatatttgaaatgtataagtccataaaatagaaaagaacaagtgaattgttgctatttaaaaaaattttacaattcttactaaggagtttttattgtgtaatcactaagtctttgtagataaagcagatggggagttacggagttgttcctttactggctgaaagatatattcgaattgtaaagatgctttttctcatgcattgaaattatacattatttgtagggaattgcatgcttttttttttttttctcccgagacagggtcttgctctggcgcccaggctggagtacagtggcatgatcttggctcacttcagccttgacttgggctcaagtgatcctcctacctgagccttctgagtaactggaactacaggtgtgcactcctcgcctggctaattttttattttttgtacaggcaggatcttgccaccttgcccaggctggtcttgaactcctgagctcatgccatctgcctgccttagtctcccaaaatgctgggattacaggagtgagccaccatgcccggctggcagttgcatggaagagaacacctctttatggcttaccctctagaatttctaatttatgtgttctgttgaaatttttgtttttttacctttattgaaacaacaaaaagtcagtattgaaacatatcttcctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaaccttttttttttaaaaaaaaaaaaaaagaaaatctcattagtgaacttatctttgcagctgagtacttaaattctttttaaaaagataccctttggattgatcacattgtttgacccagtatgtcttgtagacacgttagttataatcaccttgtatctctaaatatggtgtgatatgaaccagtccattcacattggaaaaactgatggttttaaataaactaattcactaatattaaaaaaaaa

SEQ ID NO: 14 - DNA, 2603 bp, Homo sapiens
  source 1..2603: mol_type = other DNA
ttcctggtgtaagctttggtatggatggtggccgtctccctacagactgggagctgttagagggcagggatcctagctgacacatctatgtcctcgccttggttggaggcctccaccatggacagaggccaggccctgcccctcccaggcagcctggctccttctgctgggccctgaaggcagacgggataatgtggttggccaaggcctgttggtccatccagagtgagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaactgaaaaaaaaaaaaaa

SEQ ID NO: 15 - DNA, 3884 bp, Homo sapiens
  source 1..3884: mol_type = other DNA
atcacataccctaaagaaccctgggatgactaaggcagagagagtctgagaaaactctttggtgcttctgcctttagttttaggacacatttatgcagatgagcttataagagaccgttccctccgccttcttcctcagaggaagtttcttggtagatcaccgacacctcatccaggcggggggttggggggaaacttggcaccagccatcccaggcagagcaccactgtgatttgttctcctggtggagagagctggaaggaaggagccagcgtgcaaataatgaaggagcacgggggcaccttcagtagcaccggaatcagcggtggtagcggtgactctgctatggacagcctgcagccgctccagcctaactacatgcctgtgtgtttgtttgcagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgccccccacaggagttgtacagtccctggccctgttccctacctcctctcttcaccccgttaggctgttttcaatgtaatgctgccgtccttctcttgcactgccttctgcgctaacacctccattcctgtttataaccgtgtatttattacttaatgtatataatgtaatgttttgtaagttattaatttatatatctaacattgcctgccaatggtggtgttaaatttgtgtagaaaactctgcctaagagttacgactttttcttgtaatgttttgtattgtgtattatataacccaaacgtcacttagtagagacatatggcccccttggcagagaggacaggggtgggcttttgttcaaagggtctgccctttccctgcctgagttgctacttctgcacaacccctttatgaaccagttttggaaacaatattctcacattagatactaaatggtttatactgagcttttacttttgtatagcttgataggggcagggggcaatgggatgtagtttttacccaggttctatccaaatctatgtgggcatgagttgggttataactggatcctactatcattgtggctttggttcaaaaggaaacactacatttgctcacagatgattcttctgaatgctcccgaactactgactttgaagaggtagcctcctgcctgccattaagcaggaatgtcatgttccagttcattacaaaagaaaacaataaaacaatgtgaatttttataataaaatgtgaactgatgtagcaaattacgcaaatgtgaagcctcttctgataacacttgttaggcctcttactgatgtcagtttcagtttgtaaaatatgtttcatgctttcagttcagcattgtgactcagtaattacagaaaatggcacaaatgtgcatgaccaatgtatgtctatgaacactgcattgtttcaggtggacattttatcattttcaaatgtttctcacaatgtatgttatagtattattattatatattgtgttcaaatgcattctaaagagacttttatatgaggtgaataaagaaaagcatgattagattaaaaaaa

SEQ ID NO: 16 - DNA, 11434 bp, Homo sapiens
  source 1..11434: mol_type = other DNA
cggaggcggagctcccgcgccgttcacgtgacgggcccggctgtagcggcggcggccgcggcgtcttaagcggcgcccagtgcaggatggtgctggaggcggcggcggccgtggtggcggcagcgtcgttggcggcagcgggagtgggtgcggcggcagcggcggcggcgcccgcgggtggtataaaatggcggatttcgaagagttgagggatctccagtacaatgttgaatagaagctgtgatgggggcatcctgatcttgttcctgattttaaagggaatgctttcaaagtttcacaagtggctgtaccattttacatccctaccagcaatgtatggagtgatccagtttctcagcatcctcgccagcatttggtgttatcagtgtttttcattttagccattctgaatatggtttctagttttagggtttctgaactacaagtattactaggctttgctggacggaataaaagtggacgcaagcatgacctcctgatgagggcgctgcatttattgaagagcggctgcagccctgcggttcagattaaaatccgagaattgtatagacgccgatatccacgaactcttgaaggactttctgatttatccacaatcaaatcatcggttttcagtttggatggtggctcatcacctgtagaacctgacttggccgtggctggaatccactcgttgccttccacttcagttacacctcactcaccatcctctcctgttggttctgtgctgcttcaagatactaagcccacatttgagatgcagcagccatctcccccaattcctcctgtccatcctgatgtgcagttaaaaaatctgcccttttatgatgtccttgatgttctcatcaagcccacgagtttagttcaaagcagtattcagcgatttcaagagaagttttttatttttgctttgacacctcaacaagttagagagatatgcatatccagggattttttgccaggtggtaggagagattatacagtccaagttcagttgagactttgcctggcagagacaagttgccctcaagaagataactatccaaatagtctatgtataaaagtaaatgggaagctatttcctttgcctggctatgcaccaccgcctaaaaatgggattgaacagaagcgccctggacgccccttgaatattacatctttagttaggttatcttcagctgtgccaaaccaaatttccatttcttgggcatcagaaattgggaagaattactctatgtctgtatatcttgtacggcagcttacatcagccatgttattacagagattaaaaatgaaaggtattagaaaccctgatcattccagagcactaattaaagaaaaacttactgcagatcctgatagtgaaattgctacaactagccttcgggtatccttgatgtgccctttaggaaaaatgaggctgacaatcccatgccgtgcagtgacttgtacacatctgcagtgttttgatgctgccctctatctacaaatgaatgagaaaaagcccacctggatttgtcctgtgtgtgacaaaaaagctgcctatgaaagtctaatattagatgggctttttatggaaattctcaatgactgttctgatgtagatgagatcaaattccaagaagatggttcttggtgtccaatgagaccgaagaaagaagctatgaaagtatccagccaaccgtgtacaaaaatagaaagttcaagcgtcctcagtaagccttgttcagtgactgtagccagtgaggcaagcaagaagaaagtagatgttattgatcttacaatagaaagctcttctgacgaagaggaagaccctcctgccaaaaggaaatgcatctttatgtcagaaacacaaagcagcccaaccaaaggggttctcatgtatcagccatcttctgtaagggtgcccagtgtgacttcggttgatcctgctgctattccgccttcattaacagactactcagtaccattccaccatacgccaatatcaagcatgtcatcagatttgccaggtttggattttctttcccttattccagttgatccccagtctcacctcacccttaacagcaagcagtacgtctgtcaccaccaccagctcccatgaaagcagtactcatgttagttcatccagcagcaggagtgagacaggggtcataaccagcagtggaagtaacattcctgacatcatctcattggactaaaggaggactcacttgattctgggaatcattcatcagaactgctttttcttggatctctagtctgtggaaacgttcttttttttttttttttaataatttggtatttattgaaagtcagatggattcttttgctttctgaggggtgaacacagaagactgcaacaagaaccaaatgacatgcaaggatttttcttaattatgctgtactctcagcatcagatacattcaaccataactgtatcttcctgagagatggatttcactttcatacgttaatggatggagtctacaaatcagtgaaaaaagtttttgttaaaataaagagcaataaaattatgtaaatattcaagtaaccttttttctaattaaaaaaaaaattgtaatcactgattctagaatgacagcaacttctgtttaaccttacgtgaaggaaaaaaaatatatggaaagaacttaatgtttgctgaatgagtcctttccatggagatttgctctgtttcattgaagtaatgaagtttcagtacgtggccaaagcttggattccacttttcgtcctgcatttccacttttcttcgtgattgcatcaaagaaaaatggcattcgtgttacttggtttttttcctgcttatcatttacactttgacgttgtttgctaatgagtgctgtgtgaagttctgggatttggctactcttcattggtgattatgtttaaaagcactatgcagattctgccctgacataaagtgtgttattatgtttttagtaattgtacatttttcattctagagttttctataaatttgaggcttgccttctcaaaaaagaaactatgcagccattgaatgaaatgtctttggggtacggtgtgactggaatgtttgttagaaatttgttcacactatcaaatattgatatcttggagccagcagaagagcagattttgggaggtggtaataacaaaatttaatttcttcccaacaacttaattttctcatttattttacagaatagtagtgaaatatttgatgaaactttgtattttggtagcactacatagaaaatgtgttttagatttatgatgatcatatttctcaccaatgtaatttcagtctcagcagtgattttcaaacttagggaaagggacagcattagattttttttttttttcatttttttaaaatgatatcttacctgaaactacaaacgacaaaagagaattagaaatgtttgaattaaagtgaagaagggttgggggagatgggcctgaacccacttcctgtctcaatccatgctaccccaaacactccagggaacctctgaggttttattgggtgcactttgaaaatttctcttctatagtgtgtttgtttgattttaaatcacagagaaaactgggttttactcttagagaaacattttcatccagttttttagtttgcttcatttgacttcctaaatcatttttgagttcacaaggatttggtacttttctgtttagctttctctctctaagctttatctaccttaaaaacaaagtcctttttttaatggccagtccaaccaattgatttctcaaactgaagtgcccaggtgtggactcatcaatttccgttagaatagggacatcctacttaagagttggtgcagctccaaggagctgacttgtccttgcttggggttttttttttttttctttcaccttctcaagtttccatggcctttgtgtgttctttttatgttgatttaaattcatatggttttccacaaatcccttctttggctacattgtctccttattcaatggattatccctttgtgggggctgcttattttaaagatgttgggggggaaacaaacccaaatctacgagcagtagttgcacatagttgccagttttaccttcttagtcattagatttccaaaccatgttgcagttttttggtccagatatagtatttctttctaataaagttttatgttgctgctctaaatacagatgcaatatttattgactctgtaatcagatagaaaaaacttaacttggtttgtgtggtatgacttataaagaaatgatgtatatttgttattttgttaccctttagattgtcagagactcccccaatttaatcaacaaagttttataaagtaaatgaaaatattaatagaaattagtttatttacttggttcttataactgatatctctgtgcttttataattgtgatttgtttttttgtttttttttctattttctgtgaacagttttaatgttcggttttggtgttttacactgaaattacatataaatttttaatttatttcatacaggcaacttgcattttaaaaaatacactttgaagtttatcatcttgaaattggggcttacgttgtttatctgtcttgagcattagtactttatgactttggccttatggcaacatcatgattattaatccgtcagcctttaatgtggtcactgtttcttatccagaccctgactttctagtagtttattttgctagcccagatttctgcttaaccaaatataagggagtttcagaggggtgatcttagctgtcacctagattctgtcagccaaccagtgattgtctggaatatcttaggaaatgaactgtaattgtcagcctcctaaaatcaatttttttttttttttttttgaggctgagtcttgctcttgttgcccgggatggagtgcaatggtgcgatcttggctcaccgcaacctccacctacagggttcaagcgattctcctgcctcagcctcccgagtagctgggattacaggcatgtatcaccacgcccagctaattttgtatttttagtagatacggggtttctccattttggtcaggctggtctcgaactcccgacctcaggtgatccgcctgccttggcctcccaaagtgctgggatttacaggcgtgagccaccgcgcctggccaaaataaaatgtttttatctttcttcacttagaactatgaaggccttttcttgttctggtaaccccccacagtgtaattggtgctgaaaatagtttcgtgcctgaggtctgttgtcactcactcactttctgacaattaggtgagccacttgaggattttctggcctgaagttataaaaacttggcatctgtcctaaggtaattgtaaaggcaaaatgaaagcattgaggtgaatttaggttatatggcatttatctggtaatgtgtttcagaaaagctagaatgaaaatgtgatgtaagggatagcaagtgtaggttgattcataattcttggtataaaaccaagttttccttagtcttgaggaatttgaagaaaggtaaaatgttggattttaagcaaagtaggattgaccagagactgttctgactttgacattcagacctttcagatgttcttccttcagaccagtccaagacactcagcatttccagccaagttgtcttttttttctgccaataaagtattcattagttgacgtttgtttgtattttaagtaaagtcaattttttttaaagcattaatttaattcacgggtatgcctttcatttctctggccttcagttggtttaaaaaaaaaaaaaaaaaaaaaagcacttagccggaccattctttctttagtaaacatttttattttcttaatttttcctgaaagattttcttcattttcatggaatgtcataatgacactaccattattttctagctctttgcataatagaaaaatgtagagcagtgcaccatttaaacacgtctgacattggatttagtggtcctaggttcatgtgatttgggagccttaagttgttatttagattgattcagcctgattcaccatcttttttactacagttctacatgaatgtggtgaaacaggtagaagaaaattaagcatttctctgcttttggagtctaatgttcctgcctagtgactggtttgtaacacccccctcacccctactttgttaggaacctgcaattcctaaaagaaataaataaagctgtacccaatgtccattctttaatgcttgcctggttgaataatgtttgttttttctgtttatgtcagtagtacatgcttattgtggaaaatttagacaatacagaaaagtataacaattaattgcctatgattctgtcaccagaaaaaatcacttgatatttttgcatcttccttgccattgtctttgctacttctgcatatttttacttagtttcatcattctcagtgtaatttatatcttggggtttttccccgcaacctacaggcattattccatgtcattaaaactgtgtcaactattattaattttatggtactccattgttatggatgtgccatgtttcatataacctttacttattttgaacagtttagattatatctcataatgccacaatgaatgtctttgtgtataaattattttcttaggataaaatactggaggtggaatttctagtcaagggtatatgaacacttttgtagttcttgattcataccacttttccacatgggtatgttaatttatactgcagctagatggtatgggaagggtggtctagccatattttccaagaacatgcaacgttttatctttatgcaggcatctgcactaagaccctctgcctttctgactctatggaacggggttaactgatatggagatcattggcagagattttcccaagctttcttagttttaaatatatctgacatgggccgggtatggaggctcacgcctgtaatcccagcactttgggaggccaaggcgggcagataatctgaagtcaggagttggagaccagcctggctaacatggaacatggtgaaaccccatctctagcaaaaacataaattaggtgggtgtgatggcaggcgcctgtagtcccagctacttgggagaccgaggcaggagaatcgcttgaacccaggaggcggaggatttgcagtgatccgagattgtgccactgcactccagcctgggtaacaaagcaagactctctaaaaaaaaaaaaaaaaaaaaaaaaaaatatatatatatatatatatatatatataaaatctgacatgtttcagaaacacctacgtatggctatccaagcttttcccatgattttactggcactgtaaagttgcacaaatattctttgtcagactacgttttccatctttagttcaaaagttttgtcactttttaaaaaaagcctcttgtacctaaggcctgcccttaagaggtcagctctctttttcctcttcctgtaggccccattatggcctttctgagctccccaaagccagtagaatctactgagccaaaggcattccatccccaaactgaaaagtggtaaggctaaaagctataaaaacaggcaaacattgcctgttttcactttatattcccttatgtttccaataagggtagctgcagggaaagaaaaaaacagattgctaccttcagatgtttgaggtacaacattttcttgattggcgtagcacaatcatttcccaaactgcttcctatttctgaatggcttttttaaaaagctttattttgtgttctcaaagcttaattattttaagtgacgaagagtgttaccgtgttgtagaatagcatatgcctcttattttggccttcatcaaactgtttaataaatgtttagagtgttttgtaagtacacgtggaatagagtgttacagaagttttggttccagacatcaggcagcttataatcaaggttccaaaaatcaagaattcttaagctgggcatggtggctcatgcctgtaatcccagcactttgggaagccaaggcaggcaaatcacttgaggccaggagtttgagaccagcctggccaacttggtaaaaccccatctctactaaaaatacaaaaaattagggcatgatggctcatgtctgtaatcccagctacttgggaggctgagggatgagaattgcttgaacctgagaggcggaggttgcagtgactcaggatcatgaccctgtactccagcctgggtgacagagtgagaccaaaaaaaaaaaaaaaaaaaaaaaaaaaaagtaacaggcattgtgcaaagaattttccaagaatgtctcattcttgcaatggcttctcagaggaaagtggtgttattccccattttacaaatgaaacaggtttaggtaagtttcccaaggtcacagtgaatgagtagtggacttgggagtctgacctagacttaccccaaaagctctctgcgtaaactgaagtgacgttaattagctgtgctatctaggagactgatatgggaatctagacttaaaattatgagaactggtttcagatgatggccagagtaataaggaaggagtggatgctgaagccatttcaaaggaagactcaagattcagtgactgaatataggagtaagggttgagtgcatttttcatccatttttcaagcctgggtaacttggatgttaatggcactatctacagagacaggaggcagtttagagtgaagacgaatcatgtatgggagacagtgatgaatgttaagcatgggatttcagaaatgaagcatccatgaggctcattcctggtttttctgcagtcttttaaataaaagtacaaacctcccttgtcatccatttttgccttcacttatctccattctgtattatgttctagtcacgccccagtcactgatcccttcctaggccatacttggctacatcttagccttttcttttgatttttctcttcactgccttgcccattacaaatttggctgccacctgaggccctgccataccctacagctcacaccagcgtgggctgtgttcacattctacatatctcaagatacagtggttgctgtccttcccccctactgcatcgttacacgtctctaaaaaatctctgcctctgaagctcatgccaagaggttatactatcagctacctctagtcttataactgcctgatcattccctttgatttgctggaagctttgactcacagtgttccttgccttccttgattctggtcattattattaatatcttgaacattctatagatgatttatccaagttggtctgtgaggtattggttactttacctccagcattttcgtctactgaaccacgctttctgttttcctctttgctacttgtccagactgttctttcattattgcagccttgaagaaatcctcagtaacctatccatcctccgacatgacgtagcatagttcctttgcccattctcatcagttataacaaatgttattgccctcttttcaggccacttccttctcagaagatgactttgctgcttaattcaaacagaagggacagtccgaaagcatgatttcttccccacctatgaccacctccatccttttttgtttccagtttcagagaatgaggtaggcctttaaggccaactgactctggtctcctcctgtcatcttcaggaccttcctctgtcagacttcccctctcttatctccatgtctctcacctctctactgctgcttttcttggcttacaaatggtctcagtcatccagtatttgagattatattaggtgccaggcattgtatcaggcacttggaacacaaggcaggatcctgcccccaccaaagttgtgctccaatagcgtcaacagaggtgtgaaatagggagattacaatgttaatggtaaaagacaaagtatagggtgctacaagagcatgtaaatgaggcacctcaatgatttaatagaagtaatgtggagaaatgtgaggattgatgaaaagcttgtccaaaaagggccagaaatgtttagaattgctggaagtttgctaaggaatattgagaaatatgcatagatagatcgtgaagggccttgtaaaacagtgatgagacattgaagagttttcatgatcattagtagtaatgagaatttgttgagtttggcatgattatctccatattacaaatgtggaaacagattctgacaggataagcgacttacttgcccagggtcatgcagcttgtaattgaggagtcaggtttcaaatccagttttaactactttctcagaggcctttcttgatcattctatctaaaaagcaagtgttccctaactattatcccagtaccctggtttatatactcttaatacataaaataatccctgcatattgtgtgtttcccctactagattgtaagctccatgagggcagaggtgataacttttttcacttgcatcccagagcctaacatgatatgggctacatagaaggtgcccagtaaatattggtggatgaagaaatggtggataaccatttgcatttgacttatgctgactggaaattcaatacctttggaatatataatacctcagcagtaagcattcaccaagagaggaaaacagaaggaggtgcatgtttagggtaggtggtacgggagatggatgctgaggtgttgaaaggcagcatactcaccttctgaactccaaaacttacaggggcaagggccattaatgaggggtgggtagagcagtgcacagtggggataatatgggagtaggtgtattaaacctggaagtgtaagcaactccttgtagacccaccctactgtgtataaaaggctggagaaattcttcatatgatctactctaggttatgtactgaggaccaccttatctgggtcaggtacactacacagagtgctacaatataattgtaaacataatttatcctcactgtgatggctagccacctgacccaaatcattattagactcaatactttggaactggaacaaaaatcagttgacctgatgtctgaaagacatgcccacctgctatttgacaagctatagacatatcctaccaattgatgggtagtagcactgaaactatgaaagtggatgaactcatccagaggagttgatgagtgagaaaagaagactggaaaaggaacacaaggaaatcaagaagcagcagctgagactgaaggaaaataacgtggtattacagaggccaaaaaatagagtatttcaagaagcaagtggttagtggagttaaaagctgctgatacaacctaaaaaagtaagtatccattttttaaaaaactttcatttaaaataccaccatattttattgaatctaagtacctgattgtaagacattaaagaaaaattaaacctgctaattaaa

SEQ ID NO: 17 - DNA, 5085 bp, Homo sapiens
  source 1..5085: mol_type = other DNA
agcgccatgcgcagactcagttcctggagaaagatggcgacagccgagaagcagaaacacgacgggcgggtgaagatcggccactacattctgggtgacacgctgggggtcggcaccttcggcaaagtgaaggttggcaaacatgaattgactgggcataaagtagctgtgaagatactcaatcgacagaagattcggagccttgatgtggtaggaaaaatccgcagagaaattcagaacctcaagcttttcaggcatcctcatataattaaactgtaccaggtcatcagtacaccatctgatattttcatggtgatggaatatgtctcaggaggagagctatttgattatatctgtaagaatggaaggctggatgaaaaagaaagtcggcgtctgttccaacagatcctttctggtgtggattattgtcacaggcatatggtggtccatagagatttgaaacctgaaaatgtcctgcttgatgcacacatgaatgcaaagatagctgattttggtctttcaaacatgatgtcagatggtgaatttttaagaacaagttgtggctcacccaactatgctgcaccagaagtaatttcaggaagattgtatgcaggcccagaggtagatatatggagcagtggggttattctctatgctttattatgtggaacccttccatttgatgatgaccatgtgccaactctttttaagaagatatgtgatgggatcttctatacccctcaatatttaaatccttctgtgattagccttttgaaacatatgctgcaggtggatcccatgaagagggccacaatcaaagatatcagggaacatgaatggtttaaacaggaccttccaaaatatctctttcctgaggatccatcatatagttcaaccatgattgatgatgaagccttaaaagaagtatgtgaaaagtttgagtgctcagaagaggaagttctcagctgtctttacaacagaaatcaccaggatcctttggcagttgcctaccatctcataatagataacaggagaataatgaatgaagccaaagatttctatttggcgacaagcccacctgattcttttcttgatgatcatcacctgactcggccccatcctgaaagagtaccattcttggttgctgaaacaccaagggcacgccatacccttgatgaattaaatccacagaaatccaaacaccaaggtgtaaggaaagcaaaatggcatttaggaattagaagtcaaagtcgaccaaatgatattatggcagaagtatgtagagcaatcaaacaattggattatgaatggaaggttgtaaacccatattatttgcgtgtacgaaggaagaatcctgtgacaagcacttactccaaaatgagtctacagttataccaagtggatagtagaacttatctactggatttccgtagtattgatgatgaaattacagaagccaaatcagggactgctactccacagagatcgggatcagttagcaactatcgatcttgccaaaggagtgattcagatgctgaggctcaaggaaaatcctcagaagtttctcttacctcatctgtgacctcacttgactcttctcctgttgacctaactccaagacctggaagtcacacaatagaattttttgagatgtgtgcaaatctaattaaaattcttgcacaataaacagaaaactttgcttatttcttttgcagcaataagcatgcataataagtcacagccaaatgcttccatttgtaatcaagttatacataattataaccgagggctggcgttttggaatgcaatttgcacagggattggaacatgatttatagttaaaagcctaatatgcagaaatgaattaagatcattttgttgttcattgtgcagtatgtatatagcataatatacacagtgaattataggtctcaggcttacttgatttttggctattttatatttagtgtacacagggctttgaaatattaatttacataaaggccttcatatattattacgtgttatatattacgtgttataaatttattcaataaatatttgcctagaattcccaagacctttataggtgattttgttttctgggctccttaacttcataaatagctagtatcttccagcagtagtaacagtctggataacttcttccatatccctccctctttgtttttttgagacagtgtcactttgtcacccaggctggagtgcaatggtgtggtctcggctcactgcaacctccacctcccgggttcaagtgattctcccgcctcagcttcctgagtagctggaactacaggcgtgtgccaccacacccggctaatttttcgtatttttagtgtagacggggtttcactatgttgcccaggctggtctcgaactcctgaccgcgtgatccaccacctcagcttcccaaagtggtgggattacaggcgtgagccaccgcacccggcctccatatcccccttttaaaattctgtagtgtatggtaagtcatatcagatatcagacctaatttaaatttcattttagctttacaagtccaaaaacacagaatttatatattcagatactctagcactaattttagtcttaaaatattcccacgatattctgtacacaaaatgttctttttgttacaagagctgagttgcatatactgtagataaatcatattatttttgccaatttcacaaattcctctggcccatcatgtcagtcattattgagtatatgcacacattgctacttatttgattatgtatcttttaaattgattcagtgcatagaaaactatctcttacaaactttaagtgctctgatatgacttcccccccaaattttattatgaacatttttaaaaacagaaaaattgaaaaactgtttggtaagcacatgtatatctaccatttagattcagcagttgttaatgttttgtcatttgttttctctatacctatatatgtatagatacagctagttatgcatatatatgcatatatgtgtttgtttgtgtatgtatatatgcttttttccccctgaaccatttggatgttacagacatacttatcaccgtgaaaatacttcaagtatctcctacagataatgacattctcctaaaaatccgtaataccattgtaaaagtaataattccccaatatcatctaatcaagccatatttaaatttctgaagttaactccaaatttctttatagctgattatttcaaactaggatccaattaaagtttacatatgacacttggttataactctttagttggatataacattattattattttgataaaatatggaacaaatcaattctattaataagtggtcacatttgttttgggcttaaattactttttaaagatactggattttcctaagatttctgatttacactgatatttttttttgtcattcttaattgcatcacacaatagatgtaaatgaagatgtagtcacctcagataaaattggtatcgtgtatgataatattgtatcatttatatttgccttatgttaactttaagaaattgatttttttgtattaatcattttcccattgcaacagagctatattttttctattttaagaatcatattttaggattatttttggcaaatacagtgagcacttatgtaaccagatgataatgaactcaaatgtcatgatagcttgcataaatggtgactctagtagatttgactcaagcacttctagaatcatgcactgaattcaaaagaaaaatcttgctgctttttgtccagggcttgttctattcaacttctaatttgaaagctgtacaaagtaatagaagttccatttaaatatgagttcaaaactgtatttactttttatgtggccctctctttaggggattctaattttacttagggtctctaagtgcagcataatgttcctgatgttaacagaagactgtatttttaaagttacaaatttgtatatggaattaagtaatggcgctatatacgctgttgtggggaggggggaagaaaaggaggaaccaattaaataggaccttttaaaaattgttaattttgtaaactttgcttctcttataagttattgtgattcattttagttactgtgttttattttgaaaatatttaaatattgcacttctataaatagtatgataaatgcacagacaattgcagtaaattcttttttaagctaggatatttgaaatgacaacctttggttaagtgtgtcaaggttgcaacagaattttcacaatttttttgttgtttgcaaattgttactaatattgaagaggtaagggaggcaatgcaaatgatttttaatctttttttattatcttttcagcagtttatattttttgtgactttatgcaaccatatttttactttgtcttgacaactgaaagatgtataaggttttttgccagaaatgtactgtatacatagttttaagtataacagattttactgatatgtaaaaattttgccattaaaataaatgatttctcactgagaggaacttttctaccaggttggggcatatgggagcttaatatatcatatctaatttaaaataatttcactgaaataaactccattgcttttacctaatttttttcttgagatgcttttgtagtttttcagagttttagatgattttatacaaaatcctctgcctagcactgctctttttgatgttgtagtgacaccatttacattgaattaatgcttggtagcctggggctagatgtggaactccatggatctgtgttctgactggcacctttggaatgaaagaaaagtgtgtgctgtccaaattttttccccttaattctttccctcatcttctcacccataatagaaattttatttccattgtgagttctgacaagaatgaaattccacatacaacataactgtaaattgttggtaggtagaagttaatatttgtggttcatgtatattttgaccagagtatatttaagtatataatttcagcttccttgatttagaaatatgatataataaagaaaaactccatttatcatctgtta

SEQ ID NO: 18 - DNA, 5636 bp, Homo sapiens
  source 1..5636: mol_type = other DNA
aggaagtgacgtcatgcccccggccggcaggtcttttagtctttttccccctcccttactcttcgtccccggtccctcccctccccacccctttccttctagctccgacgtttgcggccgcgggggcggcggaggatatggagtaaagccagagtcagtggccaggcacgaaggcagagcaggaacagccaggaggcgtttattaggggggcggggggaaagagccccagcaccgcccctcctggaagaaggaagaggtaactataactacccaatattgcagccatggagtccatgcttaataaattgaagagtactgttacaaaagtaacagctgatgtcactagtgctgtaatgggaaatcctgtcactagagaatttgatgttggtcgacacattgccagtggtggcaatgggctagcttggaagatttttaatggcacaaaaaagtcaacaaagcaggaagtggcagtttttgtctttgataaaaaactgattgacaagtatcaaaaatttgaaaaggatcaaatcattgattctctaaaacgaggagtccaacagttaactcggcttcgacaccctcgacttcttactgtccagcatcctttagaagaatccagggattgcttggcattttgtacagaaccagtttttgccagtttagccaatgttcttggtaactgggaaaatctaccttcccctatatctccagacattaaggattataaactttatgatgtagaaaccaaatatggtttgcttcaggtttctgaaggattgtcattcttgcatagcagtgtgaaaatggtgcatggaaatatcactcctgaaaatataattttgaataaaagtggagcctggaaaataatgggttttgatttttgtgtatcatcaaccaatccttctgaacaagagcctaaatttccttgtaaagaatgggacccaaatttaccttcattgtgtcttccaaatcctgaatatttggctcctgaatacatactttctgtgagctgtgaaacagccagtgatatgtattctttaggaactgttatgtatgctgtatttaataaagggaaacctatatttgaagtcaacaagcaagatatttacaagagtttcagtaggcagttggatcagttgagtcgtttaggatctagttcacttacaaatatacctgaggaagttcgtgaacatgtaaagctactgttaaatgtaactccgactgtaagaccagatgcagatcaaatgacaaagattcccttctttgatgatgttggtgcagtaacactgcaatattttgataccttattccaaagagataatcttcagaaatcacagtttttcaaaggactgccaaaggttctaccaaaactgcccaagcgtgtcattgtgcagagaattttgccttgtttgacttcagaatttgtaaaccctgacatggtaccttttgttttgcccaatgttctacttattgctgaggaatgcaccaaagaagaatatgtcaaattaattcttcctgaacttggccctgtgtttaagcagcaggagccaatccagattttgttaattttcctacaaaaaatggatttgctactaaccaaaacccctcctgatgagataaagaacagtgttctacccatggtttacagagcactagaagctccttccattcagatccaggagctctgtctaaacatcattccaacctttgcaaatcttatagactacccatccatgaaaaacgctttgataccaagaattaaaaatgcttgtctacaaacatcttcccttgcggttcgtgtaaattcattagtgtgcttaggaaagattttggaatacttggataagtggtttgtacttgatgatatcctacccttcttacaacaaattccatccaaggaacctgcggtcctcatgggaattttaggtatttacaaatgtacttttactcataagaagttgggaatcaccaaagagcagctggccggaaaagtgttgcctcatcttattcccctgagtattgaaaacaatcttaatcttaatcagttcaattctttcatttccgtcataaaagaaatgcttaatagattggagtctgaacataagactaaactggagcaacttcatataatgcaagaacagcagaaatctttggatataggaaatcaaatgaatgtttctgaggagatgaaagttacaaatattgggaatcagcaaattgacaaagtttttaacaacattggagcagaccttctgactggcagtgagtccgaaaataaagaggacgggttacagaataaacataaaagagcatcacttacacttgaagaaaaacaaaaattagcaaaagaacaagagcaggcacagaagctgaaaagccagcagcctcttaaaccccaagtgcacacacctgttgctactgttaaacagactaaggacttgacagacacactgatggataatatgtcatccttgaccagcctttctgttagtacccctaaatcttctgcttcaagtactttcacttctgttccttccatgggcattggtatgatgttttctacaccaactgataatacaaagagaaatttgacaaatggcctaaatgccaatatgggctttcagacttcaggattcaacatgcccgttaatacaaaccagaacttctacagtagtccaagcacagttggagtgaccaagatgactctgggaacacctcccactttgccaaacttcaatgctttgagtgttcctcctgctggtgcaaagcagacccaacaaagacccacagatatgtctgcccttaataatctctttggccctcagaaacccaaagttagcatgaaccagttatcacaacagaaaccaaatcagtggcttaatcagtttgtacctcctcaaggttctccaactatgggcagttcagtaatggggacacagatgaacgtgataggacaatctgcttttggtatgcagggtaatcctttctttaacccacagaactttgcacagccaccaactactatgaccaatagcagttcagctagcaatgatttaaaagatctttttgggtgaggtgtcttacttctattttgaaggattatttcagtttcaatcatgggtgagctgatttacatctttatatagttggcttggaggaagtacttctatgggaaagtgaacagttctgtgacaggaaacatctctgtccatgccagcatagtagttgtatggacttctaaccagttgagttttttaaagcattgaggattttttcctcttaccaactcctcttcaggtttttaaagacccagcccttcccaatctcaaagagaaaaaggaaactgagttatcttgaataacataactttttaatcaaatgtttattttggcttgtggatcttggtgttatttaaaaaattgaggtgatggtcattgcaagctcatctattaagtactatatggtacacagtctatgagtcattagtcttcattttaatatgtaaaaaatcttgatgctgtattgatttgtttgcatttaagatgacagtgagaaaatgataagcataaagagaagtatcaggttatttgctttttccaaacttttcagatgaactattgtttagtacagagactgagcaaatactacaaaattcaacttaaccttcatttcattggtttaaatgcgttattaaccatcttaagtgcaaactaatcattgtaaattatattttagcatggtctgcctcaaatagtaatgtatttttctgcattcacttggatatatttagaatcacttttttcctcctgtatcaaggaagaggtatgtgctgatttgtttggatatttgacaaggcactctgatgtgacttccctgactactaccttcatatttcatttcaaattcaaacttctgaggttgcagcatatatgaattgcattttcaaaagaagatttgtaagaattaaactatatttatgagtaaacttttgaggtttctgctgtattgtttcaaatgtaataaactttacttctgtaaaaattgagcagttgtatcttctgaccaccaacagattttcagcttgccatgatagtctgacctcattaattactgctactgaagttcaatttttttctaggaattttaggaaccttttgtttaaatattttaatttctattagccatttttaggaaggaaagaatcaattctcttaacaggaaacatgctttatttttcaaaacctttctctgatatttttctttaatttgctgattattcaaccacagagccttatgctataaatgtcatttgtattttaaaaaataatattccactcataaaactttaaaaccatctttcaacgaactatatatgtattatagttgctgccatagagttgatggtttttaattatctggaaccagcaatcatttaaaataaaccatattaagtttagtatgctggtattgtttattcattttatatgaatattcattgaaaatatatacacaatatatgtaatacacagcacttgattacaaaatgtaatttaattatattattgctggcagcattcagttaagagggtactttaaaaaatagaagtcagctttcacatctgatttctgtatgggctgtacttggttaacttgattttagaaaaaggactaacagaattgctaaagaaatgcatccaataaatgaaaaacagtaggaagatcaaatgtttttgtcaaatatattcacaacttgaccagattagctgtcctgtttgtaatgcaatattaatatgtcttttgggaaaaagcctacatatggaataaaataagtattgaagaatttttctttgtaacaatttagtagtcactgtttattgagaaattgttttttattttgtaaaataacatgatgttagtgttgaactcttaaacagaaagaaagcttaatataacagcttatagaacttgaactactaaatatgaaaataagtcatttgaaaaaaatacagtatgtaaaatttgttcattcgttgaggtaatggtgctatgtttttacaaaattgttcctacaccttttttctacttcaggtattttatttcaaccatttccatcaattgaactgttaccattgcctttttctgttgagaaattgcctctgaaaaatagtgctatttttcagcttaagtgttcttaagtgaatgaaattttcaaagtactagatcaccttaaaattatttcacgtactgaagacaattaagtccgttatgtttagagtagaaaatgtttaggttaaagagcatctgtcaacagaatctacaaaaaagattcccttgcatttgaattagttctctattctcctattgctaaatgtgtgatatatagagaggatgtataaaaggaaatggaaatagactatgtacttgtctggtttttgtttgtttttattttggaatgcttataagcctcctttacactgaataaggaagtagtttttgttttcttttgacctgtaaaatacctcacatggttgtttttacacatgaaagaaaaaggtatatgcgaacatacctgatatcaagaggagtatgcaccaaataaattttagctttgataaaactttccataaaaaaaaaa

SEQ ID NO: 19 - DNA, 2770 bp, Homo sapiens
  source 1..2770: mol_type = other DNA
gttgggcggggcagggagttcgtagccgcctctgggtaactcgactcgggcggccaaacctccggaggccggggacggaaggcgggcccgcagcagatcctggatccggaatctcccgggcaggagcggaatctgtcccgaaccgggtctgtgaggaactcgcgaacttggattaggaaatcccggagcccggatcgacaaatcccggaacccggaattaagatcgccaagtcccggatcgcggagcacagagcacggagtggactcgacgcggagcccggagtccggatcgcggcaccgcgggacgggacggagcgatgtcgggccgaggcgcgggcgggttcccgctgcccccgctaagccctggcggcggcgccgtggctgcggccctgggagcgccgcctccccccgcgggacccggcatgctgcccggaccggcgctccggggaccgggtccggcaggaggcgtggggggccccggggccgccgccttccgccccatgggccccgcgggccccgcggcgcagtaccagcgacctggcatgtcaccagggaaccggatgcccatggctggcttgcaggtgggaccccctgctggctccccatttggtgcagcagctccgcttcgacctggcatgccacccaccatgatggatccattccgaaaacgcctgcttgtgccccaggcgcagcctcccatgcctgcccagcgccgggggttaaagaggaggaagatggcagataaggttctacctcagcgaatccgggagcttgttccagagtctcaggcgtacatggatctcttggcttttgagcggaagctggaccagaccattgctcgcaagcggatggagatccaggaggccatcaaaaagcctctgacacaaaagcgaaagcttcggatctacatttccaatacgttcagtcccagcaaggcggaaggcgatagtgcaggaactgcagggacccctgggggaaccccagcaggggacaaggtggcttcctgggaactccgagtggaaggaaaactgctggatgatcctagcaaacagaagaggaagttttcttcattctttaagagcctcgtcattgagctggacaaggagctgtacgggcctgacaatcacctggtggagtggcaccggatgcccaccacccaggagacagatggcttccaagtaaaacggcctggagacctcaacgtcaagtgcaccctcctgctcatgctggatcatcagcctccccagtacaaattggacccccgattggcaaggctgctgggagtgcacacgcagacgagggccgccatcatgcaggccctgtggctttacatcaagcacaaccagctgcaggatgggcacgagcgggagtacatcaactgcaaccgttacttccgccagatcttcagttgtggccgactccgtttctccgagattcccatgaagctggcagggttgctgcagcatccagaccccattgtcatcaaccatgtcattagtgtcgaccctaacgaccagaagaagacagcctgttacgacatcgatgtggaggtggacgacccactgaaggcccaaatgagcaattttctggcctctaccaccaatcagcaggagatcgcctcccttgatgtcaagatccatgagaccattgagtccatcaaccagctgaagacccagagagatttcatgctcagttttagcaccgacccccaggacttcatccaggaatggctccgttcccagcgccgagacctcaagatcatcactgatgtgattggaaatcctgaggaggagagacgagctgctttctaccaccagccctgggcccaggaagcagtaggcaggcacatctttgccaaggtgcagcagcgaaggcaggaactggaacaggtgctgggaattcgcctgacctaactgctcagggatctttcttcccagccctggagcctggagggagaccaccctctgggtccttgctggggccgcagacacgtaggctggggtgaggagtgtctgctgtcaccctctactctccagctttagtcttataaatgtagtgataggattccttgttgcttggtccccaaagccttatactttttgcattggctttaattgggttcagcagatgcctcctctgcccccctgcaggcaggcccaagtaggactgctggaggctgtgctttgacattgtaagacatttccgaaccaaaggctgctgggtttgcatgtttacagactccccctggggcgagggtcagagctggctctggggagctgggctaggaagaggaggtgcagcccagactcttcctagcctttctaaaccaaagttctttgccattcctacaagcccagccttgctgctggttttttcctttcctttgggtatttgcactattttgggagcaagttttctatgtgggagccactttttttgtacaggggtaagttgggggttttcagggagcctgttaggtgcctccttcttttctttcctcaatctatgcaagcggctctggccgccatcatctcctgggatgccagagggctgcctctccagcggcttgggccggggaggggacactccagttctctagcatggcctgaggtatggggtatgtgcatgtggaggccagggtaaggtgaatggggaggctgggaggactggtgttgccctttggagcttggtgaggagggtgggcctagggcttggcgagtgccacatctggcaggtttggaaatttccaaataaatccttttgtctattg

SEQ ID NO: 20 - DNA, 7523 bp, Homo sapiens
  source 1..7523: mol_type = other DNA
gtccgggttcgcttgcctcgtcagcgtccgcgtttttcccggccccccccaacccccccggacaggacccccttgagcttgtccctcagctgccaccatgagcgaccaagatcactccatggatgaaatgacagctgtggtgaaaattgaaaaaggagttggtggcaataatgggggcaatggtaatggtggtggtgccttttcacaggctcgaagtagcagcacaggcagtagcagcagcactggaggaggagggcagggtgccaatggctggcagatcatctcttcctcctctggggctacccctacctcaaaggaacagagtggcagcagtaccaatggcagcaatggcagtgagtcttccaagaatcgcacagtctctggtgggcagtatgttgtggctgccgctcccaacttacagaaccagcaagttctgacaggactacctggagtgatgcctaatattcagtatcaagtaatcccacagttccagaccgttgatgggcaacagctgcagtttgctgccactggggcccaagtgcagcaggatggttctggtcaaatacagatcataccaggtgcaaaccaacagattatcacaaatcgaggaagtggaggcaacatcattgctgctatgccaaacctactccagcaggctgtccccctccaaggcctggctaataatgtactctcaggacagactcagtatgtgaccaatgtaccagtggccctgaatgggaacatcaccttgctacctgtcaacagcgtttctgcagctaccttgactcccagctctcaggcagtcacgatcagcagctctgggtcccaggagagtggctcacagcctgtcacctcagggactaccatcagttctgccagcttggtatcatcacaagccagttccagctcctttttcaccaatgccaatagctactcaactactactaccaccagcaacatgggaattatgaactttactaccagtggatcatcagggaccaactctcaaggccagacaccccagagggtcagtgggctacaggggtctgatgctctgaacatccagcaaaaccagacatctggaggctcattgcaagcaggccagcaaaaagaaggagagcaaaaccagcagacacagcagcaacaaattcttatccagcctcagctagttcaagggggacaggccctccaggccctccaagcagcaccattgtcagggcagacctttacaactcaagccatctcccaggaaaccctccagaacctccagcttcaggctgttccaaactctggtcccatcatcatccggacaccaacagtggggcccaatggacaggtcagttggcagactctacagctgcagaacctccaagttcagaacccacaagcccaaacaatcaccttagccccaatgcagggtgtttccttggggcagaccagcagcagcaacaccactctcacacccattgcctcagctgcttccattcctgctggcacagtcactgtgaatgctgctcaactctcctccatgccaggcctccagaccattaacctcagtgcattgggtacttcaggaatccaggtgcacccaattcaaggcctgccgttggctatagcaaatgccccaggtgatcatggagctcagcttggtctccatggggctggtggtgatggaatacatgatgacacagcaggtggagaggaaggagaaaacagcccagatgcccaaccccaagccggtcggaggacccggcgggaagcatgcacctgcccctactgtaaagacagtgaaggaaggggctcgggggatcctggcaaaaagaaacagcatatttgccacatccaaggctgtgggaaagtgtatggcaagacctctcacctgcgggcacacttgcgctggcatacaggcgagaggccatttatgtgtacctggtcatactgtgggaaacgcttcacacgttcggatgagctacagaggcacaaacgtacacacacaggtgagaagaaatttgcctgccctgagtgtcctaagcgcttcatgaggagtgaccacctgtcaaaacatatcaagacccaccagaataagaagggaggcccaggtgtagctctgagtgtgggcactttgcccctggacagtggggcaggttcagaaggcagtggcactgccactccttcagcccttattaccaccaatatggtagccatggaggccatctgtccagagggcattgcccgtcttgccaacagtggcatcaacgtcatgcaggtggcagatctgcagtccattaatatcagtggcaatggcttctgagatcaggcacccggggccagagacatatgggccataccccttaaccccgggatgcaaggtagcatgggtccaagagacatggaagagagagccatgaagcattaaaatgcatggtgttgagaagaatcaggagagggatacaagagaggagatggggtcccggcacccatctgtatcatcagtgcctctttgaaggtgggaaacattagtgaaaattctgttggtgccacgctttgatgagcatttgtttgaccccagtttcttcttacacttcttaccccagcctacccttcctgcatttctcttctcagctcttccatgatggattcccccccctttcctaaagccatcatgccttgataaatatatatgatcattgaaatactttttaacaaaaaacagattctatattattatatatatatatatatatataaagatatatagagatgcattcacaggggttggctgggaggaggaagaccattctgtgaccaaaataccttggtcattttttttatattgccttatttccctatggctgagccttgttgtgacacatcaagcttttctgtagatgttgtcttggcttcccaccagcttaagcgttcatatgctctgcttttagttcatatatacatacataatgtttttcctttcttaattttgtctttttgtttgggatcagcttcttgcactccttccctaactcaactgttgccgtctcatcttctctcatctgatcacttcatgttttgtttttgttactgcctggatgaggcacttctgtcaattttttcaggaccttagttccagcagcagaatggaaaaatccttgaagcccaggctgatgcttgaagtaactgtggagggagtgttcaaaatactactgacgcaggcaccttcttggcgctggagagtcaaaggcatctcccttcattagctgctctgagcatcaagaattagaagtctttcagtggaattgtacaagagtccctttgaagataataatcttggctcagtttgtataaactgtcaaattttcaaataataggtagggggctttcactaggaaaatcatgtgctcagaagaggaaatgactcgtagtcaggttcaggagttagtggagtatttggactttggtactgctgtcttccaaggtagctctaagttttgatgtgtgggcttctgagtttatattctgaaaggaaatacacttcttttgaacatccccactaggttcttttccattgtcaataaggagcatcagccagtgaatctgtttcaggtttccattctgcagaactcctccaaagcatgtgctagtggcaagacagtggttcttatgatgttttcccttaacttttccttgtatgttcttgggtggttcctaagggaaagggaagcacatgatcatgggaatgatagcccagaacaaaaagaaatcttgtcttaccacagtgttttataggagagattgggagaaatcatcctgttttctctgtgacctgatttcagaagagactgatccaaaaattataacggcagggaacctagtgcatttggcactgagatttaaatgcaaccagaattgtcctcaaggcccagccataaaagcattgtctctctcgaccttctggtatcttgttagagagcttttcactgtgaggaagtgtggaaaaatagctctgtgtgtgtgtgtgtgtgtgtgtgtgtgtgtgtgtgtaatctgttaggttggggataggttttctgctagccaatattaaaagagacctgcaataaaaaaattaccctgatctgatagaaagcaagtgtttttgtatgtgtgggtgaatgtgtgttcatgcccgtatatgtctacacacagatgacaaattatatttgaaatcgttggaaaataaattcagatcaaaatgcctttcaggcccattacctagaaatctatcttaaaacctgggtatgttcctaaggtcatttctttgcttatgctaaattaattacaattatgaatggaggatattctactgtacttttttaaaaagaaactatttttgtgtttgaaagtgaaaccaacatccagatctatagcagagtccttattcttctcataaatctttttactttggctacaaatagatgatggtatgattctattatatattttatataaaatccatccaaattaagttttgggtaagtgtgttgtttaatctgaactatagtaacttaatactctaaacaatagttcactccatttggtcctttctccacagatgtaattatgttttcaactcaggaactatggcaaggaactttccccagatcaaattctattaacgctgagatacaagtcatccatgcacagccactatcataccctttattctcactgaaaggcagaactcagaacctgttattttatgtctgtaatcatgtactttggcatcttttggaggaaaggggcaggataactcactggaatgtacagtattttgctagtgcatttcaaggaatggaatcttctccagtatgaaattaccagatataaaataatgtaatgatgctgaggatataagcttttagaaggtaatttgatggtatttctttctcgaatgaaaagctgctggtttaccctcaaccctattcattagcattaccatgagtgaatttatatctaattatttccacttgccctgttctcttcacaccaaggaagctccagatccagtatcttgtttggcctcaaaacagaagcagcttcttttgtctcccagcagtagtgagccactcagtctcttccacaggaagtttggagcctacattccttgagtcaggagcttattacagaaaaaccccgtttccctgaacttttggctaacagaaattaatttaactgacatgcatattgattctgaaatttttttcctaagtttttttcatttttttgaatgagttttttaaattttttagatgaccaaaacttgcagggcaggggatgcccagaagagtggtgagatagtaaaacacttattccctcatcctttcaggttttcaggttgcccatttatattcatttacatgtcatttgactgtctcactttttacccagaacagtaacaacccacaccgtcttccttcagggatttccaactggcactctgtgggtgctacacagaatgcaatttaatggatatttctcagcctggttcagaataaattgatcctttgatcccagaaagtatatactgaagtgtgggataaagattatgattaggggagggttggagacaaaagctgtaaattactatggctgatttatttctactatatacatatatattttttgcttttgtatatcctatataggaaactaagcattgtattttttttaacaaatctaaaaaagcactatgaactacaggtgtttgactttcaaaatatattttgtattgttaatatcttcacattgtgtgaatactggaagctgcagatctttgctaggacgcaataaatttatatactttttgaggggttcttctggggtgctaatcaggcccctgttatgcttagggggagccctggtgctacttgcttgaagttttcagtgtaagtaccctgatgccttttggaccttgggatcagatcaagagttttggagatcaggtaccaaggaaataaggacagtctagctgcctcaagtgaggggccctttgcatagctctccttccccctcactgaagctgggtagcctattggggttgagagggaaaatgtgaaatctcagaatttatctcccttagaagagagccagtaacttatgtacaaggatgaaagaaaggtcgcagcagtagctttggggaaagggaggaagatatggcacttctccaaccccggaaaacattgcttttgaaaactgctgataaaatatgagccggttattacttctgtttgggagactgtgctctctgtggtgcctctcttggctctactccacagataccagacctcttctaagaggatgagcagaccagctttgaggttgacctgtttctctttgtctgccttcccaaaacaccagcccccaggaagacattaagcagccttaagcttaaattcctactccctcttccaaatttggctcacttgccttagatccaaggcagggaaaggaaaagaaggggggtctctggctttattactcccctaagtctttactctgacttccccaaacccagaaagattttctccacagtgttcatttgaaagaggagtattttgtcccattttccccttcctcattatcaaacagccccagtcttccttgtctctgctaagaaagtagaggcatgatgatctgcctctcaactgccctaagtcctagctaagtatcaggggaaaaaaaaaaaaaaaaagcctaacaaatgggattagactagggctgcaagtagtgaggattttgttgatacctctgctgggatgtgtgctttcccatatcttgccttcaggaattacactgtgccttttccccagggatatgggctctgtctacccagtgctccagtttcccggtaactgctcttgaacattgtggacaagggcaggtcttcatatttttgatcatccctttctcccagtgaaatcccatagcccttacctagagtctagggcacaaagacttcggggaagatacactgagattgacctgaggagacatctacacacaccagtggcagctgccccagggcctgcttccccttcctaagtctgtcatcctctggaagggatgggtggtgctccaatctctggtgcctaaaaacccaagtttatttctctcttaacactggcaataaccagtccacaccactgttgccttttaaaacctcttaataatctcatgctgtgtttgttttgattccaatccaattatcaccagggctgtgtgggtaaatgcttttaaatgctctctcatcttgttcttccccctcaccccccactcttaggtatgtatgatgctaatcttgtccctaagtaagtttcttcctgctccttttgtatcttcctttcttgtctttcctcctaccttttgtctcttggtgttttgggacttttttttttttttttttggccttttgtacaaagattagtttcaatgtagtctgtagcctcctttgtaaaccaattaaaaagttttttaataaaaaaaaaaaa

SEQ ID NO: 21 - DNA, 1651 bp, Homo sapiens
  source 1..1651: mol_type = other DNA
attttgtggagcgccagagctgctaagtgcgtcagttgtggagtggcgtagacgagttaagtcctggtctgcgtggaggtcgacgactccgtcgcagactacggacctgtctgggtctcagccgccaaagaccccgtccggtaggtgagtggctcactttgagggcaagccttctcggatcgaggcttcttcatggccgctcagatcgtgagcggccggggctgctctctttgcggaggatggcgtctaatgagcgcagttgattcgaggaagtactagccggacatcatgagtggctgtcgggtattcatcgggagactaaatccagcggccagggagaaggacgtggaaagattcttcaagggatatggacggataagagatattgatctgaaaagaggctttggttttgtggaatttgaggatccaagggatgcagatgatgctgtgtatgagcttgatggaaaagaactctgtagtgaaagggttactattgaacatgctagggctcggtcacgaggtggaagaggtagaggacgatactctgaccgttttagtagtcgcagacctcgaaatgatagacgaaatgctccacctgtaagaacagaaaatcgtcttatagttgagaatttatcctcaagagtcagctggcaggatctcaaagatttcatgagacaagctggggaagtaacgtttgcggatgcacaccgacctaaattaaatgaaggggtggttgagtttgcctcttatggtgacttaaagaatgctattgaaaaactttctggaaaggaaataaatgggagaaaaataaaattaattgaaggcagcaaaaggcacagtaggtcaagaagcaggtctcgatcccggaccagaagttcctctaggtctcgtagccgatcccgttcccgtagtcgcaaatcttacagccggtcaagaagcaggagcaggagccggagccggagcaagtcccgttctgttagtaggtctcccgtgcctgagaagagccagaaacgtggttcttcaagtagatctaagtctccagcatctgtggatcgccagaggtcccggtcccgatcaaggtccagatcagttgacagtggcaattaaactgtaaataacttgccctgggggcctttttttaaaaaacaaaaaccacaaaaattcccaaaccatacttgctaaaaattctggtaagtatgtgcttttctgtgggggtgggatttggaaggggggttgggttgggctggatatctttgtagatgtggaccaccaaggggttgttgaaaactaattgtattaaatgtcttttgataagccttctgctcacatttttgtgaatgtctgaagtatatagtttgtgtatattgacagagctcttttataactaaagcaaatttaatttttttgtactagaaaaaaatttgaacattttagttcttggttataaaaatgttaattcagaattagtttaatgccttaattaaactaattaatagctttggacacttaaaagagctctaaatttgcttgtacataaaggcttaatttgttttccttgttagggtcaagggtgtcctccactctttaacagctgctggacagacacattagagcagctgtttgttattgataataaaatattataaaacta

SEQ ID NO: 22 - DNA, 3516 bp, Homo sapiens
  source 1..3516: mol_type = other DNA
atactattgaccacattatgcggccaacgatacccattttcttacctggatctttgcttcttaacactgtagtctaatttttctcgttgccttgttttcttggctgtagaagttaagaagagaaagaaggtgctgtcctggccctttctcatgagaaggctctcccctgcatcagatttttctggggctttggagacagacttgaaagcatcgctatttgatcagcccttgtcaattatctgcggtgacagtgacacactccccagacccatccaggacattctcactattctatgccttaaaggcccttcaacggaagggatattcaggagagcagccaacgagaaagcccgtaaggagctgaaggaggagctcaactctggggatgcggtggatctggagaggctccccgtgcacctcctcgctgtggtctttaaggacttcctcagaagtatcccccggaagctactttcaagcgacctctttgaggagtggatgggtgctctggagatgcaggacgaggaggacagaatcgaggccctgaaacaggttgcagataagctcccccggcccaacctcctgctactcaagcacttggtctatgtgctgcacctcatcagcaagaactctgaggtgaacaggatggactccagcaatctggccatctgcattggacccaacatgctcaccctggagaatgaccagagcctgtcatttgaagcccagaaggacctgaacaacaaggtgaagacactggtggaattcctcattgataactgctttgaaatatttggggagaacattccagtgcattccagtatcacttctgatgactccctggagcacactgacagttcagatgtgtcgaccctgcagaatgactcagcctacgacagcaacgaccctgatgtggaatccaacagcagcagtggcatcagctctcccagcaggcagccccaggtgcccatggccacagctgctggcttggatagcgcgggcccacaggatgcccgagaggtcagcccagagcccattgtgagcaccgtggccaggctgaaaagctccctcgcacagcccgataggagatactcagagcccagcatgccatcctcccaggagtgcctcgagagccgggtgacaaaccaaacactaacaaagagtgaaggggacttccccgtgccccgggtaggctctcgtttggaaagtgaggaggctgaagacccatttccagaggaggtcttccctgcagtgcaaggcaaaaccaagaggccggtggacctgaagatcaagaacttggccccgggttcggtgctcccgcgggcactggttctcaaagccttctccagcagctcgctggacgcgtcctctgacagctcgcccgtggcttctccttccagtcccaaaagaaatttcttcagcagacatcagtctttcaccacaaagacagagaaaggcaagcccagccgagaaattaaaaagcactccatgtctttcacctttgcccctcacaaaaaagtgctgaccaaaaacctcagcgcgggctctgggaaatcgcaagactttaccagggaccacgtcccgaggggtgtcagaaaggaaagccagcttgccggccgaatcgtgcaggaaaatgggtgtgaaacccacaaccaaacagcccgcggcttctgcctgagaccccacgccctctcggtggatgatgtgttccagggagctgactgggagaggcctggaagcccaccctcttatgaagaggccatgcagggcccggcagccagactagtggcctccgagagccagaccgtggggagcatgacggtggggagcatgagggcgaggatgctggaggcgcactgcctcctaccccctcttccacctgctcaccacgtagaggactcaagacacaggggcagcaaagagccactccctggccacggactctctcccctgcctgagcgatggaaacagagcagaactgtccatgcttctggggactctctggggcacgtgtctggcccagggagacctgagctcctcccgctgaggaccgtctccgagtccgtgcagaggaataagcgggactgtctcgtgcgacgatgtagccagccggtctttgaggctgaccaattccaatatgccaaagaatcgtatatttaggagggaggccatacgccatgccatagcttgtgctatctgtaaatatgagacttgtaaagaactgcctgtagattgtttttaaaaggtcttgaataagctccttgagaaagttgtggaaagccctcctcagtgaggatagctacaccatggccatggcgcatcagatagtctctgtgtacctggatttgtgcaatatgtaaaaatgtatcaaatgtattatagataaggtgttaggtgcaaaggatgtctaataatccctgcacacgttttgaacttgcagtgaagtacactgctgttccttgcttcctggggcacttttctcttggttagtgtttaaaaattatcttcgcttttttaatgtggcctcaaatgtcatgccaattttcacatcttccacaaactccatttagggagaaatgtttaaatctctggtataagtttactccataccagagtaaactatatattactctatataagcagtcttgcaataactaatcaccaccatagaagaaagaaacagactgcaaggaacagagttgagtgtctggagtcatcaaaggcattaaaaactccagtaaaagctggggccgtagcaaaaatcatgaaaaacacttcaacgtgtcctttcaatcatccaattaaatgtgggtagattaatgaaaatgtattacatcaatattaactcatctatagcactttgagtatctttgtagttcatgatatcctatcctataatgtggaggtaaatgattttatatgcattgggggtcatatataaaacttcaatgtaatttcactacaataaattgccttccttatttgaaagtaaaaatgttctttttttattgacatgttaactcctttccctcaattaatagaaatccactaagaaaatcattcacatattggttcactcaacaagcatttattaaatatatattcactattctagactaatagcaagaccggggatcttgtttaggaagaagcagtccctgttctccagaatttgcaaaaccattaaaaaagcacctactttaagccattttttttcagcaaggagtcattctgccagaaaaatgtagtacacaaatacaggataatataacaaatgtaaaatttctcatttctagtgaattaaactttccagtaattttacatctcacctcattcttatgatgctcagtttgcttaattattggcaaacataatggtaaaatgtttgtactgtattagagctttactgttcgattattaagatatttatccagtatcttagagatgctggacttcaattttccttattttatctttttaaaataaaatatcatgcatgcttctgatttgttatagaaa

SEQ ID NO: 23 - DNA, 2060 bp, Homo sapiens
  source 1..2060: mol_type = other DNA
actcggccaggcgccggcgacctgaggggagagggaacgcagctgaaagcgtgaactgtacctgtcatttactccatcctttatagtgatgctacaggacgaagaggaatggataaaaacattggcgagcaactcaataaagcgtatgaagccttccggcaggcatgcatggatagagattctgcagtaaaagaattacagcaaaagactgagaactatgagcagagaatacgtgaacaacaggaacagctgtcacttcaacagactattattgacaagctaaaatctcagttacttcttgtgaattccactcaagataacaattatggctgtgttcctctgcttgaagacagtgaaacaagaaagaataatttgactcttgatcagccacaagataaagtgatttcaggaatagcaagagaaaaactaccaaaggtaagaagacaagaggtttcttctcctagaaaagaaacttcagcaaggagtcttggcagtcctttgctccatgaaaggggtaatatagagaagactttctgggatctgaaagaagaatttcataaaatatgcatgctagcaaaagcacagaaagaccacttaagcaaacttaatataccagacactgcaactgaaacacagtgctctgtgcctatacagtgtacggataaaacagataaacaagaagcgctgtttaagcctcaggctaaagatgatataaatagaggtgcaccatccatcacatctgtcacaccaagaggactgtgcagagatgaggaagacacctcttttgaatcactttctaaattcaatgtcaagtttccacctatggacaatgactcaactttcttacatagcactccagagagacccggcatccttagtcctgccacgtctgaggcagtgtgccaagagaaatttaatatggagttcagagacaacccagggaactttgttaaaacagaagaaactttatttgaaattcagggaattgaccccatagcttcagctatacaaaaccttaaaacaactgacaaaacaaagccctcaaatctcgtaaacacttgtatcaggacaactctggatagagctgcgtgtttgccacctggagaccataatgcattatatgtaaatagcttcccacttctggacccatctgatgcaccttttccctcactcgattccccgggaaaagcaatccgaggaccacagcagcccatttggaagccctttcctaatcaagacagtgactcggtggtactaagtggcacagactcagaactgcatatacctcgagtatgtgaattctgtcaagcagttttcccaccatccattacatccaggggggatttccttcggcatcttaattcacacttcaatggagagacttaagacacatttgaaaacagacatatcaagttctatgtgatgattttgggtttttaatactataaatacttgattgtaaactaaattcaagatcatttataggaaaatctagtttcacagctatttgaatttttttctggatttactatataactcttattttttaaaagatcattctgttctttcaaggagaaataagcctaaaagaagaaaaacaaaaaaaattctgtataaaactgtaatcctttgtattcatgtttacagtgctattactataattcaaaattatgtatgtgacttagagttatataatcataatttatgtttatttcaaatatctaagtttattgcttggatttctagtgagagctgttgaatttggtgatgtcaaatgtttctagggtttttttagtttgtttttattgaaaaatttaattatttatgctataggtgatattctctttgaataaacctataatagaaaatagcagacaacataaacatctttgtaaatatcaaacctaatacatttcttgtccagtgataaaacaactggtagaattatttaaacactttagatttttaaataatatacatggctttaatttttactgtgtgtatagctacatgatgaaattaattaaatattaagaggtaaaaataaaaaaaaaaaaaaa

SEQ ID NO: 24 - DNA, 5174 bp, Homo sapiens
  source 1..5174: mol_type = other DNA
ggagagctcgccagagcgctcgcatggcgggccggtgattgtagtcaatctggccgtattctcaggcagggtcgcccggggcggactacatctcccgggatgctgcgcggccgccccgcggaagattgtgaatatgtatcagaatgttaatgattagctgctgctaaatttggtcaaagaagtcacctacacagagcgtgttgttagagctgtgctgagcgggtgtttgggttgttggctgctttcttccccctttctcacacacttgtatattattttgaggtggtgttcgcagagtttgaaaggagagagaattaaaaaaaaaagccgcaagcgtttcactcttttatttttataatccccttcaatttggggttaaaaaaaagacaagaaaacaggaaggaagagaaataaggaaatgagatgtggtaaaagaagctaaaaggtgccttttaaaagatcgttgctgtgaagtgaaaaaaatctccagagaaaccaaaaagcaccgccgagacctcttccgaaccaaaggagtttgtgtttgcttttagggaagaagaaagatcattcattcggaggaataacaaccaattaaaagacaaataaaaaaagtttggagtgggacgcagagcgagcgagaggagctgccggcgggcggtggggcgcggagcccgcactttcccggccgggtgagcggcggccgcggcgccgggctcggcgggtgcgcctcggcggagcgaacgtcggagcgttgccttgggagacgcgcgccggacaatgcccgcggcgggccagtgacgcccgcggggaatgcggagcggcccggcagccggcacccagccgccgccgcgcgttcctgccgcccgtgtcacgcgagacccggcgggggccgggaccgcccgagccgcccctcagaccgagccggccgcctccgctgccgcggccgcctcctcttcggggtcattaaagccaatgagccgcgcgcctctgccgagcgcagccaactaaatcggcttggatgattcgcgacctgagcaagatgtacccgcagaccagacacccggcaccgcatcagcctgctcaaccctttaaatttacaatttccgaatcctgtgatcggattaaggaagagtttcagtttttacaggctcaataccacagtctgaagctggaatgtgagaaactcgccagtgagaagacagagatgcagcggcattatgtcatgtattatgaaatgtcctatgggttgaatatagaaatgcacaagcaggcagagattgtcaagaggctgaatgctatctgtgcacaagtcattcctttcctgtcccaagagcaccagcaacaagtggtgcaggctgtggaacgggccaagcaggtgaccatggcagaactgaacgccatcattgggcaacaactccaggcccagcatttatcacatggacatggtctccccgtacctctgactccacacccttcagggctccagccccctgccattccacccatcggtagcagtgccgggcttctggccctctccagtgctctaggaggtcagtcccatcttccaattaaagatgagaagaagcaccatgacaatgatcaccaaagagacagagactccatcaagagctcttcagtatccccatcagccagtttccgaggtgctgagaagcacagaaactccgcagactactcctcagagagcaaaaagcagaaaactgaagaaaaggaaattgcagctcgttatgacagcgatggtgagaaaagtgatgacaacttggtggttgacgtttccaatgaggatccatcttcccctcgagggagcccagcacattcccccagagagaatggcctagacaagacacgcctgctcaagaaagatgccccgattagtccagcctctattgcatcttccagcagtactccctcctccaaatccaaagaacttagccttaagagggatatggggaaattgagtgaaacacgtcttagcgaagatgaacaatgcacattggggttacagagatggttttgtcgcctgtggtttatgaatgaaaaatctactactcccgtctcaaagtccaatacccctactccacgaactgatgcgcccaccccaggcagtaactctactcccggattgaggcctgtacctggaaaaccaccaggagttgaccctttggcctcaagcctaaggaccccaatggcagtaccttgtccatatccaactccatttgggattgtgccccatgctggaatgaacggagagctgaccagccccggagcggcctacgctgggctccacaacatctcccctcagatgagcgcagctgctgccgccgccgctgctgctgctgcctatgggagatcaccagtggtgggatttgatccacaccatcacatgcgtgtgccagcaatacctccaaacctgacaggcattccaggaggaaaaccagcatactccttccatgttagcgcagatggtcagatgcagcctgtcccttttccacccgacgccctcatcggacctggaatcccccggcatgctcgccagatcaacaccctcaaccacggggaggtggtgtgcgcggtgaccatcagcaaccccacgagacacgtgtacacgggtgggaagggctgcgtcaaggtctgggacatcagccacccaggcaataagagtcctgtctcccagctcgactgtctgaacagggataactacatccgttcctgcagattgctccctgatggtcgcaccctaattgttggaggggaagccagtactttgtccatttgggacctggcggctccaaccccacgcatcaaggcagagctgacatcctcggcccccgcctgctatgccctggccatcagccccgattccaaggtctgcttctcatgctgcagcgacggcaacatcgctgtgtgggatctgcacaaccagaccttggtgaggcaattccagggccacacagatggagccagctgtattgacatttctaatgatggcaccaagctctggacaggtggtttggacaacacggtcaggtcctgggacctgcgcgaggggcggcagctgcagcagcacgacttcacctcccagatcttttctctgggctactgcccaactggagagtggcttgcagtggggatggagaacagcaatgtggaagttttgcatgtcaccaagccagacaaataccaactacatcttcatgagagctgtgtgctgtcgctcaagtttgcccattgtggcaaatggtttgtaagcactggaaaggacaaccttctgaatgcctggagaacaccttatggggccagtatattccagtccaaagaatcctcatcggtgcttagctgtgacatctccgtggacgacaaatacattgtcactggctctggggataagaaggccacagtttatgaagttatttattaaagacaaatcttcatgcagactggacttctcctcctggtagcactttgctctgtcatcctttttgttcacccccatccccgcatctaaaaccaaggatttcagatactcattgcagttgtggagtttaatcccctttcttaacctcacttcccacttgctattgaattgtgaatagtcattaaaaacctgtgataccaaatcttcagctgtctacttggaagaacatggaataagcatacttaacagtgaaaagaatctttaattatgtattatatctgtaatatatttattttgtttaaagaaggctttctaacaatgactgactaaataaagctgtctgctcctgcattgataatgaaggtgcgttgtatttgatacccctccccccctttttttggcaaaggaggggaaaggaaggtttaaaataattgatttaaaatgtcactaagtgtagactgatgactgtatagagatgtgaaatgtataattacacatggaagcaatatgttgctgtgttgttattaggttttttttgtttttgttttctacatcttttaaagacttttggaaatttggctgaacaattagaacacaacaggccaactcatactcatttggatctatttagacaacgttaaccaatatatctatagctttagattatattcgataaaagtaattggactttttttctttttttgactcgttgacaagtgtctttgtaatatgtttttagttccctttttttgttgtattataggcagatgaacaaattaaatttggcctcaaagagagaacttactcccttctggatatttttgccacatttctttgcaaaaggagatatatatatctttagtcagttttgttgttatgagaaattatgggttattttgtggcatgctctttgggagctgcacagttatggggaggactcccactgctgtgcaagttaagtcttttacaaaacaaggacagcagaggagggtttgcagagacctccctctgaaaaacacaaagaatggactctctcctgggatgaggacttgctttctttacctccggttctttccatgtcttagttggatgtccctgaaatggacacaggctgtgccattgtgccagaaacattgtgttatcttttatgttgttgttgttgctgttaaactataatatgtgacttctttttttattattttttgtttgaatgctttaaaaatcttttaagtctgtggatctgctgatgtacagtgcctttgctgctatggatcaaaatcaaaagaaccgtgtagatatactttattgtataagtagaaaattacttaatttcatactagaaatggatggatgctgcaagttgaaatggactgtccattgacgttcctaatgtggtagcagaaaaaaaaaaatggtgtcttaagtgcttagtgtttgatgtcattaacagtttcgtaaaactctacagtgtagaaagattttgatactaaactgtgcgttgtacatagttctaatgcattgtattgaccaccagtacttctataatggtagattgtttgtgaattcagacttttaagcattaaacataaataacttctagtatgcttatttttctaattctttgtcttgatgacattagtttattttttatctttggctgtgccactcctatatattaaaaatgcctagttttttcaagggagattgttgttaaagtaaagtggttttttttgttgttaaa

SEQ ID NO: 25 - DNA, 4814 bp, Homo sapiens
  source 1..4814: mol_type = other DNA
cttcgtgccacgtcaccgcctgcgtcgcttccggaggcgcagcgggcgatgacgtagagggacgtgccctctatatgaggttggggagcggctgagtcggccttttccgcccgctcccccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgtgatgcaaaagaaagaaatccctgcgctttctgtctgtctttgtggcggcccagattgaattggggaatacatctttagcctggaaatgtaggctgcatgttaatggtaatgtaacttttgcagtgtaatgtttgaaaaatattaatgtagtttttgcttttacagtaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagagaatgccttttagtttaatgcatatttaaactaaattgatcctgtagtgttcctggagaagctagagcctgattgtaggctactactcatcaattaacttctacagtggagactacttctgggactggaatataaaaaagaatcaaaggttctgattttgagttgcaataaagggaaagaccatgctcatagcagtgccaacatctgaagtgtggagccttacccatttcatcacctacaacggaagtagttaactggaagagattaccaagagaataaaaagagactcattcagtggaagcaactttgtctcagcttatttcacataaagagagcgaagtcttttgggatgaatgttaattaaactccctggtaactagaacagggactggcaaactagcctatctgaccacctgttttgtacactttaaggtggttggttgcctttttaaatggttgaggggaaaagaataccttgtgggatatggaatttaagttcgagtccagttttattggaacgtggctatgcttattcatttatggattgactgtggctgttgtcagtgcatgagcagagttgtgtctaacagactagagcctgcaagtttgccagcccctgatttaaaagatgaaggtacacagaatgtgggctggctggtgggcaaaggggtaaaaatgttctctatattgtatctgaaaagatggggtgtctgaataagaaaatgcatctatttgacagacctggagcagttgctatctgctgctatggtttccaccacagatgcaagaagaacatgtccttgcgctttccgtctgtctaattgtggcagctgagattgaatagaggaatacaggaggaaaaaaagcgggaagagtttttgaggcaggtcggtcacccaggcttgtagtgcagtggcacaagcaactcactgcattctctgcatcctgtgctcaagccattttcccacctcagtctcactagttgctgggactgcaggcatgcacccctatgcccagctaatttttgtagagaccgagtatcgcttagttgcccagggtggtctcaactcctgggctcaaggagatctgcccacctcagcctcccaaagtgcaggcctagcctgggaggggaattttcaaaacgtgagttttgggaaatagtctatcagccttacctggttgattacacttgtaaaagaaagattaaaagcaggccagtgactctggtctgcttgaacatgtgaatgtagtggtttgagcaatctggagtttgccctagtgtcaaattccagactgtccatagtgtccaaaacctgaggcagatactaatgttaacccccagcaccccgtgattggaaacaaacctaaatacgtattgggaacttaatagcaattttaagcattctgatagattttttgtagggatggggtcatgccatgtggcccaggctggtctgaaaactctggcctcaagtgatctcaagctttggccttctaaagtgttgggattacaggtgtgaggcattgcacctggcttagcgttctgatttgacattgtaatgaaaagtgtgagtctcatctacagggccttttgtcctctgaaatgatagcaggaagggaattttcaggcagtggtcaaagctggggaaaccaggatagtgaagaaggccttgaggtgagagatggaagctaattggtgaactagccttggaagcctgaaacagacaagtagcaattcagagactttgtgggctccactgctccaacttgttttgaagattttcagttctgcagaagaggtatttccccagttgtcctttcagtgctcttagctgttttcccaacatccagatccaatcaaggctgggacatagcattttatcatgtctatttaagtcagaagtgatgaaccccagctgtttacctcatggtaaacctttgaagattccaggtagaatcttctcagactttgaagactgtctcattttatatctttttctcgttattcctagggtcaagacgttttgggcaagaataaggatgtgaacatcagaaagctcataacattttgtttttgatgctaagtttaacaaaggcatgctttagtagcctgtgggccctagggtttgttaaagtgtggagaacaactgagtggagcaagaggacttttctaggaaggtccttgtaatgtgacatttgaaaacaaatgaaggtgtggaagtaggccatgtggatatcaggacaaaccattccaggccaagacaacagcagttagtctggagtgtgatgtgttctgggaaaaaagtggccactttgctaacccaagaagacaggaagggttgtaaagcagtgggagtgtgcaaggaaggaagaccagacctcaaggaaaccacaggcgctctgagcagaagagttacatgatatgactcaaatttttaaaggatcactttggctgccaggtggcagggtaaaagcatagaataattgtgtataatgtgtttttaaggcaaagatagtggcttagtctagggtagtagactgaggtggtaggaaatgaagatagagacaacaggatatgctggtgggtgaggatggatttaatgttgatacaagtattttggtctgagcgtttggaagaaagttggcactgaggtgggaagtcgagtttagttttgttagttttggatgtgttaagtttgagatgctgattcttcagagaagtctaagctggagaactatatagagagtggaaagataacaatagacattgaaagccatgatacaggataaggtcatttggagagaggatagactgcattccaacatgagattggttgacaaagagaaaccaacaaaggtaattaagaggtgctcccactgcacttgtactcagaaggctgaggtaggattgttagaggccagcctgggcaccacagggagaccccatctctaaaatttagccaggaaccatggctcatgcctgtagccccaggaatttgggaggctgagtggggaggatcgcttgaggtcaggagtttgagaccagcctgggcaacatagggagacctaaaaaaattaattgggcatctgtagtcccagctactcaggcggctgagctgagaggatggcttgagtccgagagattgagggtgcagtgagctgtgatcataccactgcactccagcctgggcggcagtgagacactatctgaaaaaagtttaaaaattttaaaaaagaaggaactgcccctgaggtaagaaccaagggagggcctcccagaggtcaggtggaaaaagttttaggaaggaggaagtagtcaacagggttacctgttgcaaagtacttaagtaatatgaggcctgatagtggtaaacttgactaccgttggatttcactagtgggaaaggaagtctaattaaaatgcactcaagagactaacagtcgcaggcatgaaatacaatacaggtacatggttttttattatgtgtgcatctgcttcagtaataggtgtgaattactcatttggatcattaggagtttcaaaatctagttaaatgactagatttttgttgatgtaaattctgtcattctgaactgcagggattgtcagtaacttaactgcaaactaaactggtgataattatggtaaaattgcaagacgagcaataaatctcaaccaacttgagagaacactgataa

SEQ ID NO: 26 - DNA, 1781 bp, Homo sapiens
  source 1..1781: mol_type = other DNA
cgccgtgaaaaccagctgctgcagccgcagaggcagccggaggcagaggggcggcgggcaggaccagacagggctgggcagggggctggccgagcgccgtgcgccgcttgggagaaggccggaagcttaccagccgagaaggaattcctagctagcttcagagccggtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaagtggctctgtcctcagggtgggcagagccactaaacttgttttacctagttctttccagtttgtttttggctccccaagcatcatctcacgaggagaactttacacctagcacagctggtgccaagagatgtcctaaggacatggccacctgggtccactccagcgacagacccctgacaagagcaggtctctggaggctgagttgcatggggcctagtaacaccaagccagtgagcctctaatgctactgcgccctgggggctcccagggcctgggcaacttagctgcaactggcaaaggagaagggtagtttgaggtgtgacaccagtttgctccagaaagtttaaggggtctgtttctcatctccatggacatcttcaacagcttcacctgacaacgactgttcctatgaagaagccacttgtgttttaagcagaggcaacctctctcttctcctctgtttcgtgaaggcaggggacacagatgggagagattgagccaagtcagccttctgttggttaatatggtataatgcatggctttgtgcacagcccagtgtgggattacagctttgggatgaccgcttacaaagttctgtttggttagtattggcatagtttttctatatagccataaatgcgtatatatacccatagggctagatctgtatcttagtgtagcgatgtatacatatacacatccacctacatgttgaagggcctaaccagccttgggagtattgactggtcccttacctcttatggctaagtctttgactgtgttcatttaccaagttgacccagtttgtcttttaggttaagtaagactcgagagtaaaggcaaggaggggggccagcctctgaatgcggccacggatgccttgctgctgcaaccctttccccagctgtccactgaaacgtgaagtcctgttttgaatgccaaacccaccattcactggtgctgactacatagaatggggttgagagaagatcagtttgggcttcacagtgtcatttgaaaacgttttttgttttgttttgtaattattgtggaaaactttcaagtgaacagaaggatggtgtcctactgtggatgagggatgaacaaggggatggctttgatccaatggagcctgggaggtgtgcccagaaagcttgtctgtagcgggttttgtgagagtgaacactttccactttttgacaccttatcctgatgtatggttccaggatttggattttgattttccaaatgtagcttgaaatttcaataaactttgctctgtttttctaaaaataaaaaaaaaaaaaaaaaaaaaaaaaaaaaaaaaaaa

SEQ ID NO: 27 - DNA, 4360 bp, Homo sapiens
  source 1..4360: mol_type = other DNA
gcggccgcggcagggctgggcctgcgactacccgaggaggctgacctccagcccgggcgcccggttcagcgccgccccggccggcgccggtgcctgccaggcactcagggaggcgggggcgcagtggaggaggcggcgccatcgcgaagcgagcgcctcgcccgcactcagccttgccaccccgcccgcagtccaggctggactgggcggcatttgccgaggctcctcggccaggccccgtccgcccgagccgcgctgagacccgggcagcggccgcgtggagaggaggtggcagcggcccgggaggccggagccaagccagcgacccaccatggagacccgctacaacctgaagagtccggctgttaaacgtttaatgaaagaagcggcagaattgaaagatccaacagatcattaccatgcgcagcctttagaggataacctttttgaatggcacttcacggttagagggcccccagactccgattttgatggaggagtttatcacgggcggatagtactgccaccagagtatcccatgaaaccaccaagcattattctcctaacggctaatggtcgatttgaagtgggcaagaaaatctgtttgagcatctcaggccatcatcctgaaacttggcagccttcgtggagtataaggacagcattattagccatcattgggtttatgccaacaaaaggagagggagccataggttctctagattacactcctgaggaaagaagagcacttgccaaaaaatcacaagatttctgttgtgaaggatgtggctctgccatgaaggatgtcctgttgcctttaaaatctggaagcgattcaagccaagctgaccaagaagccaaagaactggctaggcaaataagctttaaggcagaagtcaattcatctggaaagactatctctgagtcagacttaaaccactctttttcactaactgatttacaagatgatatacctacaacattccagggtgctacggccagtacatcgtacggactccagaattcctcagcagcatcctttcatcaacctacccaacctgtagctaagaatacctccatgagccctcgacagcgccgggcccagcagcagagtcagagaaggttgtctacttcaccagatgtaatccagggccaccagccaagagacaaccacactgatcatggtgggtcagctgtactgattgtcatcctgactttggcattggcagctcttatattccgacgaatatatctggcaaacgaatacatatttgactttgagttataatatggttttgtgacttatgagctgtgactcaactgcttcattaaacattctgcattgggtataatctaagaattgtttacaaaaagattattttgtatttacccttcattcctttttttgatccttgtaagtttagtataaatatatctagacattcagactgtgtctagcagttacgtcctgcttaaagggactagaagtcaaagttccttgtctcactatttgatctgctttgcagggaaataacttgttttttctcatgtttcatcttctttttatgtaaatttgtaatactttcctatattgccctttgaaatttttggataaaagatgatgttttaagttccaatgagtattactagttactcaataccacttattgagtactctgtttctacgtatgtagaatgtatagggatagaagagttgaaaagggaaagcaaaactcctcaagtagcttccttaaaatgtcattcataggagatgtactggaattgctcattctgtgactttatttgtgtcctaaacattcttcagtgaaaataattttatttcagtcaaacatttatgaggaaatgagatcacatctttgtcactggatgctacttgaagagggagtactttgtaaccactttgatatgctgttatcaccaccccctgccctctgctgccataatcacacaaatttaaaaagaaagaaaacagtcttccatagatttttaaggaagaaagggcccaagccaggagatcgcttggttttcttccagaagttaaatggggggatctgaagatttgaatgtttggtctgctttgaaatgtatgtcttttgggatgtattatatgcctagctttataatcagtataaattttaattattccaggaatatgcataatattgaaatatttcatgtcctattttaatagaaaacctcagggcccaagtaacagtgatagaagttagaaaaacctttacttagaattgtccacctagtcagagcccaagaaagaattttcagtggaaaaatcaatatataacttagtgctagctagcgccacagactctagtagataatattatcatcataatggctggtgaaaccatataatcacagaaaaacattgccttcagcatgttcagttcgcagcactgagggcactcttgagggtgttgttaatgaagatttaatttttaaatacaggtggttccaagctttcaaataggttatgctccaaaagtgttatttgtaagttaatttttttacaagtcaaacaatgttggaagtggtatttaggttctagatcggtccacgaaagttagcccatatgtatatcttgaatagtataggggagggtattcataaagtccttatgtggttttaactaagtgaaattatggacaagagaaataattgtaaaatcgtcttaaaggaaaatttaatttttactcctgtttatgggacattcgttctattaactgtcagacacaatttctgttttcatctgagagccagttttcctttatttctacatctaaaataagaacatattgtacactattatataatacagaattgtcttaaactttaataaattcgcattttaaaggtgtttacagattattttttatatctgtagctgaatttgttaaagtctaaaaagctcaaggactttatgaagatctcattatatgaggaaaatcataggttaccattttataactctattgccataagaaaatacactctaaaatcttgatttgaaacatattagaaaccttgattcagtgctcagtggtctcctagtaagaagtcaccgacggtagcgtcatatgagaagaaagaaatccccaccacctcaacctctgctgagattgtgtgctaggaacagccttccctccgtttcccctcagtcaaacttgagccagcctctggatcgatgtgatcttattgcatgtttccatggggtgtacctatactttaagccaatcctgctgcattcactgctaagttaaataaaaagccaagaagattttgcactgtgcagatcctttgctatctgacttgcatctcttcccccacctgtcagctagccacctgcttgtttgtgttgggatattttttagcacctgaagcaccatctgaaaggggcaccattttcttcttccctttgatctcacatatgctccctaaaaatccttaagttgtcaatctgatccccagtgtgaggttaatgagcaaaattggtctttggggccctttttgtccaagccccactgaaaggcctcttcagaaaactattatctttaaagccctactttaactccttaattccagcatacagctaaaactggatgtatattctggcaagtaaaggctgaggactcctctttaatcctcagatctagataactcatgacattttatttgaccaacatagcacatgatgagatatcaaggtaattaaaatagcatgcttgaaaaaaaaatacgtaatctgtttcacctgtaactgtttaagccaataaacttttcaaaatttatgtaatgtggggcttttatgtagcactttacgttttcatgctgcttattgttttattctactgaaaaaaatgaatttcaagattctcaacttttttaatttcaaaaattgtttattgttttgactataggaatacaaaatttcctattttgggagaataagaactctttttgtcatttttggctatgaataaactttctggtcttttgagaccacccatttttatagatcagaatcagaaaacaggtaaacctcactcacacatttggactcatttgaacaaaaatctaggccaaaatactgaaaagcctatgtgtttttttaattggaagtatatgtaaggttaatgcatttagtgaacgtgactaacaaagactaatgtgcacattaacagatgtactttttaaggttttatgggaggctgtgcattgctcaaaagctgttgggaacgccttctgaacagttgccttcagaactagtttgagctgctcaataaaaccagtgactttactcataaaaaaaaaaaaaaaaaaa

SEQ ID NO: 28 - DNA, 6137 bp, Homo sapiens
  source 1..6137: mol_type = other DNA
gtcttttgtccctcggcggacaccgtttgccagccaaagctatgtctgcgcgctcaccgacttcatagggtgccgaattcttttttccccaggcttgccatggctagtcgaggggctcggcagcgcctgaagggcagcggggccagcagtggggatacggccccggctgcggacaagctgcgggagctgctgggcagccgagaggcgggcggcgcggagcaccggaccgagttatctgggaacaaagcaggacaagtctgggcacctgaaggatctactgctttcaagtgtctgctttcagcaaggttatgtgctgctctcctgagcaacatctctgactgtgatgaaacattcaactactgggagccaacacactacctcatctatggggaagggtttcagacttgggaatattccccagcatatgccattcgctcctatgcttacctgttgcttcatgcctggccagctgcatttcatgcaagaattctacaaactaataagattcttgtgttttactttttgcgatgtcttctggcttttgtgagctgtatttgtgaactttacttttacaaggctgtgtgcaagaagtttgggttgcacgtgagtcgaatgatgctagccttcttggttctcagcactggcatgttttgctcatcatcagcattccttcctagtagcttctgtatgtacactacgttgatagccatgactggatggtatatggacaagacttccattgctgtgctgggagtagcagctggggctatcttaggctggccattcagtgcagctcttggtttacccattgcctttgatttgctggtcatgaaacacaggtggaagagtttctttcattggtcgctgatggccctcatactatttctggtgcctgtggtggtcattgacagctactattatgggaagttggtgattgcaccactcaacattgttttgtataatgtctttactcctcatggacctgatctttatggtacagaaccctggtatttctatttaattaatggatttctgaatttcaatgtagcctttgctttggctctcctagtcctaccactgacttctcttatggaatacctgctgcagagatttcatgttcagaatttaggccacccgtattggcttaccttggctccaatgtatatttggtttataattttcttcatccagcctcacaaagaggagagatttcttttccctgtgtatccacttatatgtctctgtggcgctgtggctctctctgcacttcagaaatgttaccactttgtgtttcaacgatatcgcctggagcactatactgtgacatcgaattggctggcattaggaactgtcttcctgtttgggctcttgtcattttctcgctctgtggcactgttcagaggatatcacgggccccttgatttgtatccagaattttaccgaattgctacagacccaaccatccacactgtcccagaaggcagacctgtgaatgtctgtgtgggaaaagagtggtatcgatttcccagcagcttccttcttcctgacaattggcagcttcagttcattccatcagagttcagaggtcagttaccaaaaccttttgcagaaggacctctggccacccggattgttcctactgacatgaatgaccagaatctagaagagccatccagatatattgatatcagtaaatgccattatttagtggatttggacaccatgagagaaacaccccgggagccaaaatattcatccaataaagaagaatggatcagcttggcctatagaccattccttgatgcttctagatcttcaaagctgctgcgggcattctatgtccccttcctgtcagatcagtatacagtgtacgtaaactacaccatcctcaaaccccggaaagcaaagcaaatcaggaagaaaagtggaggttagcaacacacctgtggccccaaaggacaaccatcttgttaactattgattccagtgacctgactccctgcaagtcatcgcctgtaacatttgtaataaaggtcttctgacatgaatactggaatctgggtgctctgggctagtcaaagtctatttcaaagtctaatcaaagtcacatttgctccctgtgtgtgtctctgttctgcatgtaaactttttgcagctaggcagagaaaggccctaaagcacagatagatatattgctccacatctcattgtttttcctctgttcaattatttactagaccggagaagagcagaaccaacttacaggaagaattgaaaatcctggtactggatggctgtgataagctgttctccacactctggcctggcatctgagaactagcaagcctctcttaggccatatgggcttctccaccaaagctgtttggcagctcctagcagaccttcttattgaaatcctcatgctgaaaatgaacacagcctagttgccaacccacatgtccttttcacctccagcaagactaagcttctttaaagcacttcacaggactaggaccctgtcctggagctatctcaggaaaaaggtgaccatttgaggaactgtgacctaattttattataatgatgcctctaattttcatttcctttacaaccaactgtaactataaggttgtattgcttttttgttcagttttagcatgctattttttgaattctagactcctccatgtgaagatatcaacagacaaaactacaactgtataggacatatttggagaaaattctatcaattgatacatttggatgacatcacatttttaagtaatgtaatctgaggccattgctgaggaaattaagaattttcctttttttttaaccacccccagtgaaaaggatcagtgtatatttatagcacctattttttagttctgtctgttgtgaggcacatcctgcatggggcacttctagtcaaataggcaatgataaggacctaattaaaatgtgataagtgtatactattactttaaaagcctttacagtcagtacttcagtttacaaggcactttcacagcatctcgtttgatcctcacagtcacaacatgtggtagacaaggcaggtgatttttatccccattttacagataaggaaacaggctgcgggtggggagtgaggggaggtaaagatagttagttgcctaaggtcacacagccagtaagtaatagagctgggactggaacccaggtttccttactctcatctattgctcctccatattcctcactcaaccatgaaaacattacttgaaaggactgatgaggttaaccagagacctaactgatattgtaactttctattttaaggaagaattgtgtctgtatttgagttctttggagcctccagtctgcctgtgtgttagaccagcacagcagtgctgtgtgatgcagcctgacctgtggcaggaaagtagtgcttctgtttggaagtcatgttcttttgcagccacacaggatccaaatatcagtactattcctgtagtcaatctggggtcacattataggtgccttatttccctaagggtaactgatctgaatatctgcaaataggatgaatctatttttcagaagttccatctttcatttttctttttttttttgagacagagtctcattctgtcgcccatgctggagtgcagtggcgcgatctcggctcgctgcaacctctgcctcccaggttgaagcaattctcatgcctcagccacccgagtagctgggattacaggcatgcgccatcatgcccagctaatttatgtatttttagtagagttggagtttcaccatgttggccaggctggtcttggactcctgacctcaggtcatccacccgcctcagcctcccaaagtgctggtattacaggcgtgagccaccgcacccagccccatctttcattttcaaagagaagggcattctaataggaactggtgccaagagagaagaaaagaagtgataacagaagaaatggctagttacaatattaaaaagctcctctttgagatctcctctgcaggaatatcagagacggagttgaagcgctggagaggtaataggtctagacagtacagaacaataactggggagtgtgtgaggatagactgggctcccccttgcttgaaagatctctggcatttaattctcaattcttgattactattttccagtgtaaaactagcacatatgatctgactacaggacagagaattttaagtgaaacatttgccttacttgcagtaataatgtgctgttcttcacagtagctaaggccctctatgtttcccagaggtaaataagaatccaggaatggaggtccatctgtgatgaatggcttttttctaatcaaagtagtataatgctgttttatctgttttgtcatcttgtttttttttttttttaaaaaaacaaaaccttaattataatatagcgcaaagaaaggccaggactgatgcagggattccttggaaatatcagttcctatcacttttaaaacctgattttggatctctctgttctatgtatgtctttagtgagagcacaatacatggcagaacgctgtgccaaatgttataggtaaggaatatagaaatgaatgttttttgttgtgaaggtgttttcatgtgatattttataaacacattttaaaaaatctccatcactttttagtataggaaggatagctttgcctgggaaaaacagtttcaacacacctgctcagagtagcagttctccctcaaaaaagcagtgttcagcctgcactgactgttctgcttgccaaaaggaggaagcatgcaagatacttatttctccatagattgtggagtatagagggatgtgggactacagattattattttttttccccgagacagagtcttgctctgtcgcccaggttggaacacaatggcacgacctcagctcactgcaacctctgtctcccgggttcaagcaattctcctgcttcagcctcctgagtagctgggattacaggcacacaccaccaccgcactcagctaatttttgtatttttagtagaggtggggttttaccatgttggccaggctggtcttaaactcctgaccttgtaatcatcccgcctcggcctcctaaagtgctaggattacaggcatgagccaccgcacccggcccagataatttttaatagcctttgatcatggggtgagtgagggagtaggtatacttggcaaatgcatggttctctgatttctagctctaaagcagccttatctgaatccccaaatcttgtgatgctgagtaccattactgaaccagtctgcacggtaggcatctgctaccaaaatttacctcctacctggtaggtgtcatctgataagaaagaagacaggttattttaattttttgagataatcacagaaaattgcagcccatactctttattaccgaattcaagtttggaaatagaccctttgttttaaatcatgatgggtctttatcccaatcatttatctgggtcatttttccaactttggagttctaggaaagaaccttgaaaacctgatatgattctgcagcatgaggtctacggtgaccatttgggcaaagctccagtggcaatcatttattgtgttttgcatttcctgggatttattgaaataagaattcactgtgattatgtagtcttctggctagtatcaggcagctctgcttttaatttggttaattttattttctctgaagagggagaagaggtacaatttaatcttggcctccacaagcatattaaagctcacgtgttaatcagtgcattcttatgctcctacattaaatgccttgggtaaatggataaatggacatgtgcccagctttaattttttttgcaacagaaagatcagacttccgtatggcatcgttggatttcagaggctttctggtgtatctgtaaatctgaatgttgccttctgccagtctgtataaccaggtgattcatgctgcaaatgaaatcaggaagcagtaaagtgttaaagcaagagtattgtccaattcacttgtcttcctgatccttgtactttatttcacgtgtcggtgtttacattacatacttatatttcctgtgaaagaaagagttaaataaattgtagcagtttga

SEQ ID NO: 29 - DNA, 3531 bp, Homo sapiens
  source 1..3531: mol_type = other DNA
agcggagcgccaggcagcgcggagcggaggccaggcccacagccgctccgcctcccggcccgcagatccccgacggccgcaccgcgggctcctctggcccgcaagaacacgtgcatggcgtcctggggaaggcgctgagtgcggagtcgcggcgccgcacgcggcaccatggccctggagcaggcgctgcaggcggcgcggcagggcgagctggacgtgctgaggtcgctgcacgccgcaggcctcctggggccctcgctgcgcgacccgctggacgcgctgcccgtgcaccacgcggcccgcgctgggaagctgcactgtctgcgcttcctggtggaggaagccgccctccccgccgcggcccgcgcccgcaacggcgccacaccggcccacgacgcctccgccaccggccacctcgcctgcctgcagtggctgctgtcgcagggcggctgcagagtgcaggacaaagacaattctggtgccacagtcttgcatctggctgcccgcttcggccaccccgaggtggtgaactggctcttgcatcatggcggtggggaccccaccgcggccacagacatgggcgccctgcctatccactacgctgccgccaaaggagacttcccctccctgaggcttctcgtcgagcactaccctgagggagtgaatgcccaaaccaagaacggtgccacgcccctgtacctggcgtgccaggagggccacctggaggtgacccagtacctggtgcaggaatgcggcgcagacccgcacgcgcgcgcccacgacggcatgaccccgctgcacgccgcggcgcagatgggccacagcccagtcatcgtgtggttggtgagctgcaccgacgtgagcctgtccgagcaggacaaagacggcgccaccgccatgcacttcgcggcgagccgcggccacaccaaggtgctcagctggctgctgctgcacggcggggagatctcggctgacctgtggggcgggaccccgctgcacgacgccgccgagaacggggagctagagtgctgccagatcctggtagtgaacggcgcggagctggacgtccgcgaccgcgacgggtacacggccgccgacctgtcggacttcaacggccacagccactgcacccgctacctgcgcacggtggagaacctgagcgtggagcaccgcgtgctttcccgggatccatccgcagagctggaggctaagcagccggattcaggcatgtcctcacccaataccacggtgtcggtccagccgctgaactttgacctcagctcgcctaccagcaccctctccaactacgactcctgctcctccagccactccagcatcaagggccagcaccctccatgtgggctttccagcgctagagctgcagacatacagagctacatggacatgctgaacccggagctgggcctgcctcggggcacgattgggaagcccacacccccaccacccccacccagcttccccccgccacccccgcccccaggcacccaactgcccccacccccacctggctacccagctcccaagcctcctgtaggaccacaggcagctgacatctacatgcagaccaagaacaaactccgccacgtggagacagaggccctcaagaaggagctgagctcctgtgacggccacgacgggctgcggaggcaggactccagccgcaagccccgcgccttcagcaagcagcccagcacgggggactactaccggcagctgggccgctgccccggcgagacgctggccgcacgcccgggcatggcgcacagcgaggaggtgcgtgcccgccagcccgcgcgcgccggctgcccgcgcctcggccctgccgcccgcggctcactcgaaggcccctccgctcccccgcaggcggcgctgcttcctgggaaccatgttcctaacggctgcgccgcggaccccaaggcgtccagggagctgccaccgccgcccccaccgccgccgccgcccctgccggaggccgcgagttcgccaccgccggccccgcctctgcccctcgagagcgctggccctggctgcgggcagcgccgctcctcctcgtccaccggcagcaccaagtctttcaacatgatgtccccgacgggcgacaactcggagctactggctgagattaaggcaggcaagagcctgaagccgacgccccagagcaaggggctgaccacagtgttctcaggcatcgggcagccggccttccagcccgattcgccgctgccttctgtgtcacctgcactgtcaccagtccggagccccacaccgccagctgcggggtttcagccgctgctcaatggaagcttggttcccgtgccgcccactactcctgcgccgggagtgcagctggacgtggaggctctcatccccacgcacgatgagcagggccggcccatccccgagtggaagcgccaggtgatggtgcgcaagatgcagctgaagatgcaggaggaggaggagcagaggcggaaggaggaggaggaggaggcccggctggccagcatgcccgcctggaggcgggacctcctgcggaagaagctggaagaagagagggagcagaagcggaaagaggaggagcgacagaagcaggaggagctgcggcgggagaaggaacagtcagagaagctgcggacgctgggctacgatgagagcaagctggcgccctggcagcgacaggtcatcctgaagaagggggacatcgctaagtactagaggccgcagactcctgtccgcagcctcgcagctccgtggggccctccgccccagccccagccagccaggccctggtggaaaggctgggagccgcacagccctcccctcctgcgctggaaaccctccctgacccccaccctggccccccgtatccccagcccttggcaacactggagtgcacacgccgccacggttgcccagaaaaagtgcccaagctgctgacgcaaacaacaacaaatgctgcttatttgcatgccgacttacatatatttgcatgttcgttgactatcaaagagtgcagagctctccccagccccgtgggtggtgactttgttttcctgcggggctcagccccctccaggatgcagccccctcccccgcaccccggaaccggcgtcgctggcgcatcctgggtggaggcaggccccgagctcggggaaggggttttcccttcctctctgacccagatctgcgcgcggcctagcccgggcctcatttcttatccccgccaagggtttcctctcagtcatttgtttaccagaaacatgaaaactgcctgtctggccgggccgcacttgtggcccccgggaccccacctctggccccacctccctcaagtctgcgccccgtccccagccagacccactcgctgccgggaccctttcactgccccggtggagtgaatagaggatgaggggccctgaccctgtgtctccaactgctgcaccccatcccgaccctgtctccgccacctcgcagccccattaaagcgctctcatctgggctccggttcactca

SEQ ID NO: 30 - DNA, 2272 bp, Homo sapiens
  source 1..2272: mol_type = other DNA
ttgggcggtggaccgcccctcggccccggggtaggctgacacgggagggtcctcagctaaagccaaaagcagatcaaagtggtgggactcgcgtcgcggccgcggagacgtgaagctctcgaggctcctcccgctgcgggtcggcgctcgccctcgctctcctcgccctccgccccggccccggccccgcgcccgccatggagaagactgagctgatccagaaggccaagctggccgagcaggccgagcgctacgacgacatggccacctgcatgaaggcagtgaccgagcagggcgccgagctgtccaacgaggagcgcaacctgctctccgtggcctacaagaacgtggtcgggggccgcaggtccgcctggagggtcatctctagcatcgagcagaagaccgacacctccgacaagaagttgcagctgattaaggactatcgggagaaagtggagtccgagctgagatccatctgcaccacggtgctggaattgttggataaatatttaatagccaatgcaactaatccagagagtaaggtcttctatctgaaaatgaagggtgattacttccggtaccttgctgaagttgcgtgtggtgatgatcgaaaacaaacgatagataattcccaaggagcttaccaagaggcatttgatataagcaagaaagagatgcaacccacacacccaatccgcctggggcttgctcttaacttttctgtattttactatgagattcttaataacccagagcttgcctgcacgctggctaaaacggcttttgatgaggccattgctgaacttgatacactgaatgaagactcatacaaagacagcaccctcatcatgcagttgcttagagacaacctaacactttggacatcagacagtgcaggagaagaatgtgatgcggcagaaggggctgaaaactaaatccatacagggtgtcatccttctttccttcaagaaacctttttacacatctccattccttattccacttggatttcctatagcaaagaaacccattcatgtgtatggaatcaactgtttatagtcttttcacactgcagctttgggaaaacttcattccttgatttgtgtttgtcttggccttcctggtgtgcagtactgctgtagaaaagtattaatagcttcatttcatataaacataagtaactcccaaacacttatgtagaggactaaaaatgtatctggtatttaagtaatctgaaccagttctgcaagtgactgtgttttgtattactgtgaaaataagaaaatgtagttaattacaatttaaagagtattccacataacttcttaatttctacattccctcccttactcttcgggggtttcctttcagtaagcaacttttccatgctcttaatgtattcctttttagtaggaatccggaagtattagattgaatggaaaagcacttgccatctctgtctaggggtcacaaattgaaatggctcctgtatcacatacggaggtcttgtgtatctgtggcaacagggagtttccttattcactctttatttgctgctgtttaagttgccaacctcccctcccaataaaaattcacttacacctcctgcctttgtagttctggtattcactttactatgtgatagaagtagcatgttgctgccagaatacaagcattgcttttggcaaattaaagtgcatgtcatttcttaatacactagaaaggggaaataaattaaagtacacaagtccaagtctaaaactttagtacttttccatgcagatttgtgcacatgtgagagggtgtccagtttgtctagtgattgttatttagagagttggaccactattgtgtgttgctaatcattgactgtagtcccaaaaaagccttgtgaaaatgttatgccctatgtaacagcagagtaacataaaataaaagtacattttataaaccatttactatggctttgtaacaattgcatacccatattttaagggacaggtgaatttactactttctaaagtttattgatacttcccttttatgtaaaatgtagtagtgatacctatatttccacattgtgcattgtgacacacttgtctagggatgcctggaagtgtataaaattggactgcatttcttagagtgttttactatagatcagtctcatgggccatctcttcctcagatgtaaatgatatctggttaagtgttatatggaataaagtggacattttaaaactagcaaagttaaaaaaaaaaaaaaaaaaaa

SEQ ID NO: 31 - DNA, 4510 bp, Homo sapiens
  source 1..4510: mol_type = other DNA
gcagagggggcggagagcgcccccgggggcggggcacgcaagtgacggcggcgcgggtggtggagcgctgggcggccaggctccctggctggccggtttgggcgtctgggccgtgaaggtgggacctcctgttccgggccgcaagtttccctctccagccgcccgccgttcgtagcatgtcccccagaactcggggagcgcaggcaggacaggcttagagaagacgcggtccccagcgcttgggccacggacgtcccaccccgctcctctgtcgctggagaaccgccgggccgagccactgggagaagcaggccagagccttccagggcctccggcccgtggacccgaggaggatgagctggctttttcccctgaccaagagcgcctcctcctccgcggctgggtcccccggtggcctcaccagcctccagcagcagaagcagcgcctgatcgagtccctccggaactcacactccagattgcttcctccacagtttcctcaggaaaaaccagtgatcagtgtttatccaccaatacgacatcacttaatggataaacaaggagtgtatgttacctctccattagtaaacaattttacaatgcactcagatcttggaaaaattattcagagtctgttggatgagttttggaagaatcctccagttttagctcctacttcaacagcatttccttatctatacagtaacccaagtgggatgtctccttatgcttctcagggttttccatttcttcctccatatcctccacaagaagcaaacaggagtatcacttctttatctgttgctgacactgtttcttcttcaacaacaagtcataccacagccaagcctgccgctccttcatttggtgtcctttcaaatctgccattacccattcccacagtggatgcttcaataccgacaagccaaaatggttttgggtacaagatgccagatgtccctgatgcatttccagaactctcagaactaagtgtgtcacaactcacagatatgaatgaacaagaggaggtattactagaacagtttctgactttgcctcaactaaaacaaattattaccgacaaagatgacttagtaaaaagtattgaggaactagcaagaaaaaatctccttttggagcccagcttggaagccaaaagacaaactgttttagataagtatgaattacttacacagatgaagtccactttcgaaaagaagatgcaaaggcagcatgaacttagtgagagctgtagtgcaagtgcccttcaggcaagattgaaagtagctgcacatgaagctgaggaagaatctgataatattgcagaagacttcttggagggaaagatggaaatagatgattttctcagtagcttcatggaaaagagaacaatttgccactgtagaagagccaaggaagagaaacttcagcaggcgatagcaatgcacagccaatttcatgctccactatagattttcctggaaacatgaactgccaagagaggaatgggacacaaaaccaaacactgttttatatttatggtttgcaaactggcatttcatcagtggctaaattcacagatatcctatatagattgtatacagaactgagactgattttgtaccgattagaatgattgctatgatctttgagaaatttttctgcactatttgcactgaaatgtttatttattgttgataaattgtatcatatttaagttccactgctgttcctcttaccttgattaaatgcctatgcatgtacttttagctagtttttaatattttataaaacttcatttaaatttgtatttttaacttgaagttccatttctttatcaaggatggtatttagatttttttcctcttaaccttttttcaaaaactattttcaactgtgaggaaacccttatttttctttctttgtggataaaactttcaaaagcaatttaagatattcatagtgttaggaaacaccaaacctgcctatgtgccatctcacaaaagaaacttttaatacctacaataaatcaaaagaataaaccagctgttcttatatattgtttcatttttaaaactaaagatgcatttaagaagcaatacaagtaaatattttacctaataggaaaaaaaaaagttgcctttcatttaaaccattccaacagaaattcttatgctaatttaaaacatatatatatctggtaggtttgtggttggataggttttctaaattcctaatgttaaaaacaatctttatgttaatatacactaaatctatacacaaaaaaagtcagtgaacttttctgacctttactgtgagttaccttttcctaagaggaaagctatagtaataagtaaaatttaatttttaggcaatcctgatttttaatgaatttaattgagtgttcttgtatactacattgagcagtttgcttctataccgtgtcacaaaattcatgtatttcttgagaagccctaaaagctcataaaggaaaatgccgtgaactatgtagctcaggcttggtaaggtgccatctaaattacaaaacaaactaatgcataattttgcttaaatttcatcccagtatgattgtcttcccaacaccagcatatagtatagattgtctgtcttttttatattttttagttcttcctgtacatgtttttggcaataaagttataggaagaacaaaattattttgttagaattaaaacatgcttaatatttagtctgtttgtggagggcaggtattcacgtggactgagatacaatgttggatacagaaaataactttcattgtcttcctgacactgtgctaaggacatgctgttaaagcttcaaagtgaccagatgaggaaggaataattaattattactcctgatttgtagataactgaggtaagagtgtttcaaatttatgatagtcttttgggtattcagaaacctttccttatactgcactggccaccagagcttaattttcccagcagttacagcaatgggagatagaacagtctcaatcttttgccaaccatcaggttcctagaaaccaggtaggtgtatcccataacaagggaggagcataccacagcccctcatttgattaattcatttgatctatctatgttattaagtacctactaggaataaggcattgtggaaatactatacaaagataaacattgtttagatgcttatctactttccttttcaccagaaaaacagaaaaaaaagaaacattttcttacagagtaaaaatgttctacataatcacatgagtagttcatctcagtgttttttattctttaaagttgaactatcccagtttcattctataccattcattggataaccttgttacaacccagtcatgaaacagagcagtgtgatcagttatctgcatttaacaaatagacaaatcagtttacataaaggttatgtatgtcacccacgatgaaaagaatctgcatttgaatatgcccgtatgaatgtgggttctgtttttgcaacagagattaagtgaccattttttctaattttatggctatatattttcttcataaaaattggtcacatcggagaagcagtgccacaggaaaaatgaaatgcatgtgaaagtttgtattctgattttacaagatgagatagaaatcagaattaaagaggaatacttaggagttactaggctaatcagtgtacgaatttgtcataggtagagatttaaaggttaatatcttaaaatagaagaaaattctaaatcaatcaatcagtgagatataaactaaacagacccacttcaaagttgaaagaaatttctaggcataaattgagactaggaaatttatatcagaatagagggtgcttgacacatatatatgcttaaattgaaggacagctcagattcatttttaggagaagaaagtaaactaatgtgctcttaaagaataaaaatttattctatggtttctgtctctgatcatcaccttccattctataaaaagctcagttactgatttgctgggtcatggtcaaaattcttacctatttatttcatatcaactttaaaaaataaattacttgcattctatatattactaattgggaagtaatatgcctcaaatcagttttatactggattattccctatgctttaaaccactgctctcaataaaacacttcctgattaatgtttgattattagatattttagtcttgttggggatattttagtcttgttgggttagccatgctctgaagaatctgtgaaagtacagtaaagttttaataagcaataaatgtaaccttttatataaatctcagtgctaggttaacttctaataagcagacgaacatgttacataaattataatgtctgtcttgtaaaaaagttgaggggactaaaagtttatgactctgatatggaagttgtcatattaaaaaactacattttaaaacatcaaatatttatactatttgcttttcaaataaaagcatagtgctgtttggcata

SEQ ID NO: 32 - DNA, 11062 bp, Homo sapiens
  source 1..11062: mol_type = other DNA
gcagatcgggagcggtgccgagaaaaatttccttactagatgacatttcatcgcaatgtccgatcgtttggggcaaattaccaagggcaaggatgggaaaagcaagtactcgactctcagcctgtttgataagtataaaggaaaatcagtagacgcgattagatcctcagttattcctagacatggcttacagagtcttgggaaagttgctgcagcccggcgcatgccaccgcctgcaaacctgccaagcttgaagtctgaaaacaaaggaaacgaccccaacatcgtgatagtacccaaggacgggacgggatgggcaaacaagcaggatcagcaagacccaaagagttccagtgcgacggcctctcagccgccggagtcgctgccgcagccgggtttgcagaaatctgtctccaatttgcagaaaccgacacagtcaatcagtcaggagaatacaaattcagtgccaggtggaccaaagtcatgggcacagctgaatggaaagccagtaggacacgaaggtggtttaaggggctcaagccgactgttatccttctctcccgaggaatttccgacgctgaaagcagctggagggcaggacaaggctggcaaagaaaagggcgtcttagatctgtcgtatgggccaggaccaagcctccgccctcagaatgtgacaagctggagggagggcggtgggcgacacataatttctgccacgtctctgagcacctccccaactgagctgggcagcaggaactcgagtacgggagatggagccccctcctcggcatgtaccagcgattctaaggacccctctctccgcccggctcagcctgtccgaaaaggggcttcacagttcatgggaaatgtataccacccacctacataccatgacatgcttcctgcttttatgtgttcgccgaagtcatcagaaaaccagggtacagtggaacgaggctcttttccccttcctcagctccgccttgaacctcgagttccttttagacagttccagatgaatgaccaagacggaaaagaaaacaggctgggattgtctcgcccactccgcccactaaggcagctggtggagcgggcaccacggcccaccattatcaatgcggaaaacctgaagggccttgacgatctggacgccgatgccgatgatggctgggcaggcctccatgaagaagtggactattctgagaaactgaagttcagtgatgatgaagaggaggaagaagttgtgaaggacggcaggccaaagtggaacagttgggaccctaggaggcagcggcagttgtcaatgagctctgcagacagtgcggacgctaagcggactcgagaggaagggaaggactgggctgaagcagtgggtgcgtcccgtgtggtccgaaaggcgccagaccctcagccaccgcccaggaagcttcatggctgggcaccaggccctgactaccagaagtcatcaatgggcagcatgttccggcaacagtccatcgaggacaaggaggacaagcccccaccaaggcagaagttcattcagtcagagatgtccgaggcggtggagcgagcccgaaagcgccgggaagaagaggagcgccgagcccgggaggagaggctggccgcctgtgctgccaaactcaagcagctggaccagaagtgtaagcaggcacgaaaggcaggtgaggcccggaagcaggcagagaaggaagtgccctggtctccaagtgctgagaaggcatctccccaggaaaacggccctgctgtccacaaaggctccccagaattccctgcccaagagacccccaccacattcccagaagaggcacccacagtgtccccagcagtggcacagagcaacagcagtgaggaagaggccagagaggctgggtcccctgcacaggagttcaagtatcagaagtcccttcctccccgattccagcgccagcagcagcaacaacagcaggagcagctgtacaagatgcagcactggcagccggtgtaccccccgccgtcccacccccagcgcaccttttacccacaccacccccagatgttgggcttcgatcccaggtggatgatgatgccttcctacatggacccacgtatcacgcccactcggaccccggtggacttctacccctccgccctgcatccctcaggactgatgaagcccatgatgccccaggagtccctcaatgggacaggctgtcgctctgaggatcagaactgtgtgcccccactccaagaaagaaaagtgacccccatcgactcaccccctgtgtggagcccagagggctacatggcactgcagagcaagggctacccgctcccgcacccgaagtcgagtgacaccttggctatggacatgcgtgtcaggaatgaaagctctttctctgcctcactcggaagggcagggggcgtaagtgctcagcgcgatctctttgaggagagaggggaggagtacttgagtgcttttgacaagaaggcccaagcagactttgacagctgtatctcttctcaaagaataggccaggagcttttgtttccaccccaagaaaatgttcaggatgcaggtgctcctgggggtcacacccaaaacctcaggtgttccccattggagcctgactttgtcccagatgagaaaaagccagagtgtggcagttgggatgttagccaccagccagagaccgctgacacagcccatggtgttgagcgggagacaccccgggaggggacggcctttaacatctcctcctgggacaagaacgggagccccaacaaacagccatcctcggagcctgaatggactcccgagccccggagctccagcagccagcacccggagcagacgggcaggacccggaggtcgggacccatcaagaaaccagtcctgaaagccctcaaggtggaagacaaggagaaggagcttgagaagattaagcaggagctaggggaggagagtacccggctggccaaggagaaggagcagagccccacggcagaaaaggatgaggacgaagagaacgatgcctctctggccaactcctccaccaccactttggaggacaaaggccctggccatgccacttttggccgcgaggccaccaaatttgaagaggaggagaaacctgacaaggcctgggaagccagacccccacgagagtccagcgatgttccccccatgaagagaaataactggatctttattgatgaggagcaagcctttggggtcagaggacaggcccggggccggggccgtggtttcagagagttcacttttcgtggtcggcctgctggcggaaatgggagcggcctctgtggtgggggggtcctgggggcccgcagcatctactgcagcagtcagcgcagcggccgtggccggggcctgcgagagtttgcgcggccagaggactgccccagagccaagccccgacggagagttgccagtgagacccatagcgagggctcagagtatgaagaacttcccaagcgccgccggcagaggggctccgagaacgggaatgaaggctcgctcctggagagggaggagagcaccttgaagaagggcgactgcagagattcttggcggtccaacaaggggtgctctgaggaccacagcggtctagatgccaagagccgaggccctcgggcctttgggcgagccctccctccccggctgagcaattgcgggtatggacggagaaccttcgtctccaaagagtcaccccactggcagagcaaaagtccaggcagctcttggcaggaatatggcccttccgacacatgcggatcccggcgacctacagacagagactatgtcccagattcctacagacaccctgacgcatttggtggccggggctttgaggacagccgcgcggaggacaagagatccttcttccaagatgaacacgtggcagattctgaaaatgcagagaaccggcccttcaggagaaggcgccccccacgccaagataagccccctcgattccggcgcctccggcaagagcgggagtccctgggcctgtggggacccgaggaggagccccacctgctggcaggtcagtggccaggcaggcccaaactgtgttctggggacaagagtggcactgtgggccgcaggtcccctgagctctcctaccagaactcctccgatcacgccaatgaggagtgggagacggcctccgaaagcagcgacttcagcgagcggcgggagcggcgggaaggccctgggtccgagcccgactcccaggtggatggtggcctgtcgggggctagtttgggtgagaagaaggagctggccaagaggagcttctccagtcagagacccgtggttgacagacagagccgaaagctggagccgggagggtttggggagaagcccgttaggccaggtggtggtgacacctcccctcgctatgagagccaacagaatgggacgcctttgaaagtgaaaagatccccagacgaggccttgcctggaggtcttagtggctgcagcagtgggagtggccactccccctatgccctggagcgggcagcccatgccagtgctgaccttcccgaagcctccagtaaaaaggcagagaaggaggccaagttggctgctccgagggcaggtgaacagggagaggccatgaaacagtttgacctgaactatggaagtgccatcattgaaaattgcgggtccagccccggggaggagagtgaggtgggttctatggtgggcgaaggcttcatcgaagtcctgaccaagaagcagcgccgcctgctggaggaagagagaagaaagaaggagcaggccgtgcaggtgcctgtcaaaggtcgaggcctttcctcccgtattcctcctcgatttgcaaaaaagcagaacaacttatgtctggagcaaggtgacgtgaccgtgcctggcagcagcctgggcactgagatctgggagagcagcagccaggctctccctgtgcaggccccagccaacgactcctggaggaaagctgtcactgccttcagcagcaccgagactggctctgcggagcagggttttaagagcagccagggagatagtggcgttgacttgagtgccgagtctcgggagtcgtctgcgacctcctcgcagcgcagctccccatatgggactctgaagccagaggagatgagcgggcccggcctggcggaacccaaggccgacagccacaaggagcaggctccaaagccatctgagcagaaggattcagaacaaggctctggacagagcaaggagcacagaccaggacccatcggcaacgagcgttctctgaaaaacagaaagggctcggagggggccgagcggctgcaaggggctgtcgtcccgcctgttaacggggtggagattcacgtggactccgtgctgcctgtgccacccattgaatttggagtcagtccaaaagactccgatttcagcttgccacctggttctgcctctggtcctactgggagtccagttgttaaacttcaggatgccttggccagtaatgcagggttaacacagagtatccccatcctgcggcgggaccatcacatccagagggccatcggtctctccccaatgtccttccccaccgccgaccttactctgaagatggagtctgcgcgcaaggcttgggaaaactcccccagtttgccggagcagagctctccaggcggcgctggctcaggcatccagcctccatcctctgtgggtgcctccagcggggtcaactacagctccttcggtggagtgtccatgccacccatgcctgtggcctctgtagcaccttctgcttctatgccaggcagccacctcccgcccctgtacctggatggccatgtgtttgcaagtcagccccggctggttcctcaaacgatacctcagcagcagagttaccaacaggccgccgctgcccagcagatcccgatctcccttcacacatctctgcaggcacaagctcagcttggactgaggggtgggcttcctgtgtcccagtcccaggagatcttcagctccttgcagcccttcagatctcaggtgtacatgcaccccagcctgtcaccgcccagcaccatgatcctctctgggggcacagccttgaagcctccatactcggcgttcccaggcatgcagcccttggagatggtgaagccgcagtctggctcaccctaccagcccatgagcgggaaccaagccctggtctacgagggccagctcagccaggctgctggcctgggtgcctcccagatgttggactcccagctcccacagctgaccatgccactgcctcggtacggctccgggcagcagccactgatcctgccccagtctattcagctgccacctgggcagagcctctccgttggggccccccgaaggattcctccgcccgggtcccagccgccagtcctgaacaccagcagagagccctctcagatggagatgaaaggcttccactttgccgacagtaaacagaatgtcccttcaggaggccccgtgccatcgccacagacctacaggcctagctctgctagccccagtgggaagccctctggatcagcagttaacatgggctctgtgcagggacactacgtgcaacaggcaaaacaacgagtggatgagaaacccagcctgggagccgtgaagctgcaggaggccccctcggctgcctcccagatgaagcgaaccggagcgatcaagcctcgggctgtcaaagtggaggagagtaaggcctgacagtgcctggctgccacctcgcctctccctactgaggacggtgccgccatgcggcctcgacacagccgacactcgggagcctcaccagatccaccgtccaaatgcgtggcccagactgagagacctccctcctctccactcccgaaagctccgttgtcaaccagcttgcacccgtggatatatggcattgacccgcttgctttgatacgaaacaaaaaagcagacgactccttcatcccatctgctcctaccgtgactgtggagtgacgcctcctgtgcagtgcagatttgccctccctgcctcctccctgtcctgccgcgcagccagggcgccttctcagcagtgcttccggcccagccgcccatccctaggcacagtgatttggcagcagggtcattttactttgaggctttttgttttaaaatgtagccaaggtttttacaaaggggaaaggaaaagaaaacaaaaacgcaagctccatgtgtatagctgaacttttatatgtttcttgccagcccctccgctcccttccatctctagcctctgtcctgtttagtttgatacgtcactgcagtaccttaagaggtgactcttaagaatgcatcccctcctgattcctcagctggttcacccttgaggttatttgcaaaaagaaaaggaggttcttgagggcaccgattgcgagcattctggtgcctggctccccgcctgggaagcgatggggtgctcagagcagcaggcaggttgggggagggggggggtcatagttgggttccagctcctggcttgatgagcccagggcgcttacaggcagcccatgaagttgatgacagttttagcatgagaatcacacagggtccctgtcctgggctcctctaaagccagtggatgtgctgggcaccagagacaaatcatggagatggctgctggtggctcccaggttggcccagatggggtgagctgacataccacaggcccatcccaggccccgtgggctctgcttctggggctccataccctgccctgcaggggtgctgtgtttttcacacatttctttccctgaagccttctgtaacctgtcattttccttccttcctcttccggagcctgctgctttctctggacctgtctccacctcccacacagctcatcgtgaacaccacttggtgatggagggagtggacccgtgtgtggtccccaagtgaggccactgggagtttgtccttttcctcctttgcttcactcccagcagcagacccaggttgtcaggacaggagggcctgagctaagcagtaggcatcagtctcgtttgtcttcagacggcgggggcaggtccagggtgaggctgggtggagggctgaccaaggtccaaagggcctgcgcagcctccgggagggcagcttctccagccagaggcttgtgtgagccatcgtgtgctgggcttgtttttaagtaagaaacaaggaaatcactccagattctgtcattccaaggaaagggaaggggacagttcaggtttctcagctgttcttaggggtcactgagcgtctacctcctcctccagaggaggctggctcagaacacctagaggagggggccggggatgcaccccccaccagaggctgccttcagcgtctcacgggtgcaggacagcgctcaggcttgggctctaagctctgtgtctagtgtagaacatggggaaggagcatcttaggaactgctgaagtaacttcttactgctctcacaattctaaggaagcgggagaacggcctcctaccaacagcgcccaccccagagctgcctgggaaagggcagttttactgaaaggtgctttactgttcacctgcatctttcagcagctcccctcctgccctcacctggtcttttccctctttatcccaagcctttatgcttgagtcccttccccaggggctgcccacccgacagttccaggcattccctacctgagcttcttgtctgcttttccttctcccactgcaagcggctgcttgtggggcctgggatgagccctctctgtccccaccggccctccttgccaagccattcctgggtgagttcaggcctgcgggagccacacattcatctccacctggacacttgagccgcatggccagacccctcccacctgatgcggtggtgcgtgtgatttgtcaaaagaaagccttctggatgctgttaagatgtacccttcaggtgaacctggtatcagacccacagtacttgctgtttgagaaaaaataaaaacaaaaaggtcacctgttctccagcccttttctcttacctggtatttccttcctttctcctcccccaccccaaataaaaaaacaaaaaacactagaatttatttatatgtattgatgttgtaggtctaggtgaaaaaaaaagaagtaaatgtttcactgctctatttatatataatgtctgaattaattctgtgcaggaaaggccaggaaattgcatgtgaagttcggtgcagtcaccacctgtgtgtgacctgagctgcagtctcttcgctgagatgcaggttttaaatgagacttggggggctgagggcaggcctcaggcctcccagcgccccaacccctccttggtctaatgaaatgcagttcttagtgcagagatgttttaaggtgcaatatatctcttcctttcccgtggttttagagccaagctcaaggtagtaggacgtagggtcttattttgttttcaaacccccatcctcagagcgcagatacatgcagaggcttctgccaggataccacggggccttagtgggaacaggtggagaccagcacttccctttcctgctgctgaggtagggattggggggtcagaacccactcacttttgcctgttaaagttgccctcctgacgctggcagctctgccttggtcactggggatgcggctcgttgctcagccaccagtggccttgcggtattgtccaccatccactagagtgggatgaagtccagagtgtgggtatacatctcagatgcccatctacccactggggacttcaatgccagctgcatttggtttggttttcttaactgttggcttctccccacagcgttttttgtttttttttaaacattcatattgttttcaaacttggaattcatagacactctggctctaggttccttaagggggaaaacaaaagatgactttatttcacattcaagaaaatcagttcagttccaaagctgtggtccttccagccacttctagggacactggggaaccttgttaaacgttgacatcagtgctctccagccgtgctgtcaccctcctatcttctggatctgccttcgcgatggtcagtgacagcttctggaagctgagcacacacaggtgcacagccatgctgtggtctggcctgctacggcagcatggcagctctggtggagccttctcccttgccatttggttcccctgtgccaagtagctgcaggctgcccctcaaatcttcatttgtcccttttcacttcctgcagaacaagcctgggttagagggtctgctggaaatggcctttgaagaccaaggataccaggatgtgtgcactctgtcgtgttctgtgatgaatgggaaacgtaggcttccagaaagccagctctcttctgaaatgtgacggacctaagcaggaagtcatccaggacaggagtggctcagtgttggggatggacgctgtcgcccagccatgctccaccagggccaccaatgtgtagttggctggtggtcttcgggcatgtgagacctgctcttcactgtttccaccccacttggtggcctccaggatggtagtggcaccctcagagccccatcttcagcatgttctgaagcctcagagtggaaattcctgctaaggctctgtgtggacgcctttctcccgtgatctaaaggggacactgtactcaagcttttgacctcatgccttgtgtagtaaaaaaggatttgggggttttgtttggttcctgagagggttgtgttttgtttttgtttccttttgtttatgttttggcctttcctctttgtctttccatgtagaccagatatttgaaagggcagacgatggctagaggtgtaatgtgcagcttgtttatacggtattttgggaaacttaccttggatgggaaatcgaatcgtggattcaccaggccggtgctggcacactcaccctcgccctttccctccggttcagtacctattgtttctcctttcaaatatgtgattgtactagctctttccatatgaaagaattctccttatttaaataaaaaaagtttaaaaa

SEQ ID NO: 33 - DNA, 7689 bp, Homo sapiens
  source 1..7689: mol_type = other DNA
tcccacagtgcctggcccagaagccttgctaaatatttgaacaggattgcccaatacttttctgctgtgagaatgtaagatggatccagaagagcaggagctcttaaatgattacagatacagaagctactcttcagtgattgaaaaggctttgagaaattttgagtcctcgagtgaatgggcggatctcatatcttcacttggcaaactcaacaaggctcttcagagtaacctgaggtactccttgttgccaagacggctcctcatcagcaaaagattagctcagtgtttgcaccctgccctgcccagtggtgtccacttaaaagctctggaaacctacgagattatctttaaaatcgtggggaccaaatggctggccaaggacttgtttctgtacagctgcgggttatttcctctcctggcacacgcggcggtgtcggtgaggccggtgctgctcaccctgtacgagaagtacttcctcccactgcagaagctgctcctgcccagtctgcaggccttcatcgtgggcctgctgcccggccttgaagagggctccgagatctccgacagaacggatgctctgctcctgagactgtcgctggtggttggcaaagaggtgttttacaccgccctctgggggagcgtcctggccagcccgtccatccgcctccctgcctcagtcttcgtggtgggccacatcaacagggatgcccccggccgggagcagaagtacatgctggggaccaatcaccaactcacggtgaagtctttgcgtgcctccctgttggactcaaatgttcttgtgcaaagaaataatctggaaatcgttctgtttttcttcccattttatacctgtctggattccaatgagagagccatccccctcctcagatctgacatcgtgcgcattctctcagccgccacccagaccctactgagaagggacatgtccctgaacagaagactgtatgcatggttactaggctcagacataaaaggaaataccgttgtgccagaatctgaaatctcaaattcttatgaagaccagtcgtcttatttttttgaaaaatactccaaggatcttttagttgagggtttggctgagatattgcatcagaagttcatagatgctgacgtggaggaacgccatcatgcatacctgaagccttttcgcgtcctcatcagtctgcttgacaagccagaaatagggcctcaagtggttgggaatttgtttctcgaagtcatcagggccttttattcttactgcagagatgcccttggctctgatcttaaacttagctacacccagagtggaaattcgctgataagtgcaatcaaggaaaacagaaatgcctctgagattgtcaaaacggtaaatttgctgataacttctctaagcacagactttctctgggattatatgacaaggtgttttgaggaatgctttagaccagtgaagcagcgttacagcgtgaggaacagcgtcagccctccccccacggtctcggagctctgcgccctcctggtcttcctgctggatgtcattcctttggaactttactctgaggtgcaaacccagtatctccctcaggtgctcggctgcctggtgcagcctcttgctgaggacatggaggccttaagtttacctgaactcacgcatgccttgaagacgtgtttcaaggtgctcagcaaagtccagatgcctccttcctacctcgacacggagtccaccagcggaacctcgagtccagtaaaaggtgaaaacggcaaaataattttggaaacaaaggcagtgattcccggtgacgaagatgcttcgtttccccctctgaagtctgaggacagtgggatcgggctcagtgcctcgtcaccggagctctctgagcacttgagggttcctcgagtttctctggaaagggacgacgtttggaagaagggcgggagcatgcagaggacgtttctttgcatccaagagctaatcgccaactttgccagcaagaacatttttggagtacagctgacagcgtcaggagaagaaagcaagtccgaggagcctgcagggaagagggacagggatgggacgcagagcctggcagccaatgattccagcaggaagaactcttgggagcccaagcccatcactgtgcctcagttcaagcagatgctgtcagacttgttcacagcacgagggtctccattcaagacaaaaagttcagagtcaccatcgtcttcgcccagcagccctgccaggaaaaacgggggagaatgggatgttgagaaggtggtcattgacctggggggttccagggaggaacgcagggaggcctttgccgccgcctgccacctgctgctggattgtgccactttccctgtctacctgtccgaggaagagaccgagcagctctgtgcaacgctcttccagctgccaggagccggtgattccagttttccatcttggctgaagtccctcatgactatttgctgctgtgtgactgactgctacctccagaacgtggccatttccactctgctggaagtgataaaccattcccagtccctggcgcttgtcattgaagacaagatgaaacgctataagagctctggacacaaccctttttttggcaagctgcagatggtgacggttcctcccattgctccagggatattgaaagtcattgcagagaaaacagatttctatcagagggtggctcgtgtgctttggaatcagctgaacaaagagacccgggagcatcacgtcacctgcgtagaattgttctaccggctgcactgcctggcccctacggccaacatctgcgaggacatcatctgccatgccctcctggaccctgacaagggaacaaggctggaagctctgtttagattttccgtgatctggcatctgacaagagagatccaaggcagtcgagtaacatctcacaatcgctcctttgataggtccttgtttgtcgtgctggacagcctggcctgcacggatggtgccatcggtgcggcagcccagggctggctggtgcgtgcgctctccctcggggacgtggctcgcatcctcgaacccgtgctcctgctgctgctgcagccaaaaacccagagaacctccatccactgcctcaagcaggagaactcggccgatgacttgcaccgttggtttaacaggaagaaaacctctttcagagaggcatgcgcagtgcccgagcctcaggagagcggctctgaagagcacctgcctctgagccagttcaccacagtggaccgtgaagccatttgggccgaagtggagaaggagcccgagaagtacccgctgcgaggcgagctgagcgaggaagagctgccctactacgtggagcttccagacaggacggcccacggcgccccggacagcagcgagcacaccgagtctgcagatacaagctcctgccacacggacagcgagaacacgtcctccttctcctccccttcccacgacctgcaggagctgagcaacgaagagaactgctgtgcacccatccccatggggggcagggcgtaccccaagcgctcggccctgctggcggccttccagtcagaaagcttcaaggctggggccaagttaagcctggtgcgggtggactcggacaagacgcaggcttctgagtcgttctccagcgacgaggaggcggacttggagctccaggccctcaccacatccaggctgctaaagcagcagcgggaaaggcaggaggccgtcgaggccttgttcaagcacatcctgctctacctgcagccctacgactctcggcgggtcctctatgccttctcggtgctggaggctgtgctcaaaaccaaccctaaggaattcatcgaggctgtgtccaggactagcatggataccagctccaccgcgcacctcaacctcatctccaacctcctcgctcgccaccaggaggccctcattggccagagtttctacggaaagctccagacccaggtccccaacgtgtgcccccactctctgctcctggagctgctcacctacctctgcctgagcttcctgcgctcctactacccttgctatttgaaggtctcgcaccgagacattctcggcaaccgggacgtgcaggtcaaaagtgtcgaggttttgatcaggataatgatgcagctggtctcagtggccaagtcttcggaagggaagaacgtggagttcatccacagcttgctgcagaggtgcaaagttcaggagtttgtcctgctctccctgtcggcgtccatgtacacgagccagaagcgctacgggctggccaccgcccaccacggcagggccctgccagaggacagcctctttgaggagagtctcattaacttgggtcaggaccagatctggagtgagcacccgctgcagattgagctgctgaagctgctgcaggtgctgattgtcttggaacaccacctgggtcgggcccatgaggaggcggaaaaccagcccgacctgtcccgggagtggcagagagccctgaacttccagcaggccatcagcgccctgcagtacgtgcagccccaccccctcacctcccagggtcttctggtctctgcggtggtgaggggtctgcagcccgcctacggttacggcatgcatccggcctgggtgagcttggtcacgcattccttgccctacttcggaaagtccctgggctggacggtgacaccctttgttgtccagatttgcaaaaacttggatgacttggtcaagcagtatgaaagcgaatctgtgaagctctctgtcagcacaacctccaagagggaaaacatttctccagattatccactcacccttctagaaggtctaacgaccattagtcatttttgtcttttggaacaagccaaccaaaacaaaaagaccatggctgcaggtgatcctgccaacttgaggaatgccagaaatgccattttggaagagctgcctcgaactgttaacaccatggcccttctctggaatgttctcagaaaggaggagactcaaaagagacctgtcgatctcctaggggccacgaagggatcctcttccgtttactttaaaaccaccaaaaccataagacaaaaaattttagacttcttaaaccccttgacggcccatcttggggttcagttgacagcggctgttgcggcagtgtggagcagaaagaaagcccagcgtcacagtaagatgaagattatcccaacggcaagtgcatcccagctaacccttgtcgacttggtgtgtgcactcagcaccctgcagactgacacgctgctgcacctggtgaaggaggtggtgaagaggccaccccaagtcaaagggggtgatgagaaatcgcccctagtggacattcctgtgttgcagttttgctatgcttttctccaaaggctcccagtaccagccttgcaagagaacttttcttcactgttgggagtattgaaagagtctgtacagttgaatctagccccacctgggtattttctgcttctcagcatgctgaatgactttgtaacaagaactcccaacctggaaaacaagaaggaccaaaaagacctgcaggaaatcactcagaaaatcctagaagctgtggggaacattgccggctcttccttggagcaaaccagctggctaagcagaaacctggaagtgaaggcccaacctcaggcctctctagaagaatctgatgctgaggaggacctgtatgatgctgctgcagcttcagcaatggtgtcttcatccgccccgtcggtgtacagcgtgcaagccctctctctcctggcagaggtactggcttccctcctggacatggtttatcgaagtgatgagaaggagaaagctgtgccgttaatctcccgtctgctttactatgtttttccatacttacgcaaccacagtgcctacaatgctcccagcttccgggctggcgctcagctgctgagctccctgagtggctatgcctacacaaagcgagcctggaggaaggaggtcctggagctgtttctcgaccccgctttctttcagatggatacttcctgtgttcattggaagtccattattgaccatcttttgactcatgagaaaacaatgtttaaggatttaatgaacatgcagagcagttctttgaaactattctcaagttttgaacagaaagccatgctgttaaagcgccaggcttttgctgtcttcagtggagaacttgatcaataccacctttaccttccactgatacaagaacgcctgacagacaatctcagagttggacagacatccatagttgctgctcagatgtttctttttttcagagttttgctgctaagaatatctcctcaacatttgacttcattgtggccaataatggtctctgaattgattcagacattcacacagcttgaagaagatctaaaagatgaagatgagtcattgagaagcaccaacaaagtaaacagaacgaaagtttcagtcccggatgcaaatggaccctcagtgggggagataccccagagtgaactcatcttgtatttatcagcttgcaaattcttggacacagcgctttcttttccacctgacaagatgccattatttcaaatttataggtgggcatttattccagaagtggacacagagggccctgccttcctgtcggatgtagaggagaatcaccaagaatgcaaaccccacactgtcaggattctagaacttctaaaattaaagtttggggaaatcagtagctctgatgagatcaccatgaagagtgaattcccgcttctgcgccaacattctgtttccagcatcaggcagttgatgccattcttcatgactctaaatggtgcatttaagacccagagacagctgcctgctgatagcccaggaactccattcttggactttcctgtcacagatagcccaaggatcttaaaacaactggaagaatgcatcgaatatgattttctggaacatccagaatgttaaccatgtgagagagaatatgtttaatccatgtattggtactttactgaaaaccaggttatattctaaagaagaaagaaggcaggatagtgcttttgaacaagcctatttccattttgaaagtagatttcaggctaggtgcggtggctcacacctgtaatctcagcactttgggaggccaaggcaggcagatcacttgaggtcaggagttcgagaccagcctgaccaacatggtgagaccctgtctctactaaaaatacaaaaattagctgggtgtggtggcggcgcctgtaatcccagctacttgggaggctaaggcatgagaattgcttgaacccaggaggtggaggctgcagtgagccgagatcacgacactgcactccagctgtgtgacagaatgagaccatctccaaaaaaaaaaaaaagtagatttcagataatttactgttcagcaacaggacacacctccctaaatgccttgtaatatatttgaatctgattctgcatttcttcctcaatttatgtaatgaaaataaaattaatatatcatctaacagtagcacaaaatttgtaatatgaagtaaagtatgaagataatgaagaagttgttttctttgttgaagcagttatatgggtctttctcagtatatttctcttttctctaaaagtttaaacttattaaaagaatgttatttttaacctttcaaaaaaaaaaa

SEQ ID NO: 34 - DNA, 1138 bp, Homo sapiens
  source 1..1138: mol_type = other DNA
gctctggccggccccggcgattggtcaccgcccgctaggggacagccctggcctcctctgattggcaagcgctggccacctccccacaccccttgcgaacgctcccctagtggagaaaaggagtagctattagccaattcggcagggcccgctttttagaagcttgatttcctttgaagatgaaagactagcggaagctctgcctctttccccagtgggcgagggaactcggggcgattggctgggaactgtatccacccaaatgtcaccgatttcttcctatgcaggaaatgagcagacccatcaataagaaatttctcagcctggccgaaaatggttggccccacgaagccacgacaactggaggcaaagagggttgctcaacgccccgcctcattggaaaaccaaatcagatctgggacctatatagcgtggcggaggcggggcgatgattgtcgcgctcgcacccactgcagctgcgcacagtcgcatttctttccccgcccctgagaccctgcagcaccatctgtcatggcggctgggctgtttggtttgagcgctcgccgtcttttggcggcagcggcgacgcgagggctcccggccgcccgcgtccgctgggaatctagcttctccaggactgtggtcgccccgtccgctgtggcgggaaagcggcccccagaaccgaccacaccgtggcaagaggacccagaacccgaggacgaaaacttgtatgagaagaacccagactcccatggttatgacaaggaccccgttttggacgtctggaacatgcgacttgtcttcttctttggcgtctccatcatcctggtccttggcagcacctttgtggcctatctgcctgactacaggtgcacagggtgtccaagagcgtgggatgggatgaaagagtggtcccgccgcgaagctgagaggcttgtgaaataccgagaggccaatggccttcccatcatggaatccaactgcttcgaccccagcaagatccagctgccagaggatgagtgaccagttgctaagtggggctcaagaagcaccgccttccccaccccctgcctgccattctgacctcttctcagagcacctaattaaaggggctgaaagtctgaaaaaaaaaaaaaaa

SEQ ID NO: 35 - DNA, 1377 bp, Homo sapiens
  source 1..1377: mol_type = other DNA
atgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaacttactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctctt

SEQ ID NO: 36 - DNA, 1779 bp, Homo sapiens
  source 1..1779: mol_type = other DNA
cggcgtgccctggggcggcgcgggcgcaggggcgcgtgcgcggcgggctgtcgttggctggagcagcggctgcgcgggtcgcggtgctgtgaggtctgcgggcgctggcaaatccggcccaggatgtagagctggcagtgcctgacggcgcgtctgacgcggagttgggtggggtagagagtagggggcggtagtcgggggtggtgggagaaggaggaggcggcgaatcacttataaatggcgccgaagcaggacccgaagcctaaattccaggaggttgggatgaatgggttccggagagcagagtactcaaatacgtggacaccaatttgcagaaacagcgagaacttcaaaaagccaatcaggagcagtatgcagaggggaagatgagaggggctgccccaggaaagaagacatctggtctgcaacagaaaaatgttgaagtgaaaacgaaaaagaacaaacagaaaacacctggaaatggagatggtggcagtaccagtgagacccctcagcctcctcggaagaaaagggcccgggtagatcctactgttgaaaatgaggaaacattcatgaacagagttgaagttaaagtaaagattcctgaagagctaaaaccgtggcttgttgatgactgggacttaattaccaggcaaaaacagctcttttatcttcctgccaagaagaatgtggattccattcttgaggattatgcaaattacaagaaatctcgtggaaacacagataataaggagtatgcggttaatgaagttgtggcagggataaaagaatacttcaacgtaatgttgggtacccagctactctataaatttgagagaccacagtatgctgaaattcttgcagatcatcccgatgcacccatgtcccaggtgtatggagcgccacatctcctgagattatttgtacgaattggagcaatgttggcttatacacctctggatgagaagagccttgctttattactcaattatcttcacgatttcctaaagtacctggcaaagaattctgcaactttgttcagtgccagcgattatgaagtggctcctcctgagtaccatcggaaagctgtgtgagaggcactctcactcacttatgtttggatctccgtaaacacatttttgttcttagtctatctcttgtacaaacgatgtgctttgaagatgttagtgtataacaattgatgtttgttttctgtttgattttaaacagagaaaaaataaaagggggtaatagctccttttttcttctttctttttttttttcatttcaaaattgctgccagtgttttcaatgatggacaacagagggatatgctgtagagtgttttattgcctagttgacaaagctgcttttgaatgctggtggttctattcctttgacactacgcacttttataatacatgttaatgctatatgacaaaatgctctgattcctagtgccaaaggttcaattcagtgtatataactgaacacactcatccatttgtgcttttgtttttttttatggtgcttaaagtaaagagcccatcctttgcaagtcatccatgttgttacttaggcattttatcttggctcaaattgttgaagaatggtggcttgtttcatggtttttgtatttgtgtctaatgcacgttttaacatgatagacgcaatgcattgtgtagctagttttctggaaaagtcaatcttttaggaattgtttttcagatcttcaataaattttttctttaaatttcaaagaacaaaaaaaaaaaaaaa

SEQ ID NO: 37 - DNA, 7827 bp, Homo sapiens
  source 1..7827: mol_type = other DNA
gtagtcttgacgtgagctagctggcatggcggcctgcattgcagcggggcactgggctgcaatgggcctaggccggagtttccaagccgccaggactctgctccccccgccggcctctatcgcctgcagggtccacgcggggcctgtccggcagcagagcactgggccttccgagcccggtgcgttccaaccgccgccgaaaccggtcatcgtggacaagcaccgccccgtggaaccggaacgcaggttcttgagtcctgaattcattcctcgaaggggaagaacagatcctctgaaatttcaaatagaaagaaaagatatgttagaaaggagaaaagtactccacattccagagttctatgttggaagtattcttcgtgttactacagctgacccatatgccagtggaaaaatcagccagtttctggggatttgcattcagagatcaggaagaggacttggagctactttcatccttaggaatgttatcgaaggacaaggtgtcgagatttgctttgaactttataatcctcgggtccaggagattcaggtggtcaaattagagaaacggctggatgatagcttgctatacttacgagatgcccttcctgaatatagcacttttgatgtgaatatgaagccagtagtacaagagcctaaccaaaaagttcctgttaatgagctgaaagtaaaaatgaagcctaagccctggtctaaacgctgggaacgtccaaattttaatattaaaggaatcagatttgatctttgtttaactgaacagcaaatgaaagaagctcagaagtggaatcagccatggcttgaatttgatatgatgagggaatatgatacttcaaaaattgaagctgcaatatggaaggaaattgaagcgtcgaaaaggtcttgattctgagaatgaatttggttagttgcagaagatacattggctctaagaggatatattttgagaccaatttaatttcatttataagaacatagtaattaagtgaactaagcattcattgttttattaatactttttttctaaaataaaacttgtacaccagtttattactctaaaaagagaattacacatgccaaatggaccaatgtccatttgcttattggaggcaaagctacaatagaagtcagagcatcaccagaatggtctttaatgagcatggaacctgagcaaagggaataggtgggatgaattttttttttaattgtgaaacaattcataagcacaatatgatttacagaataataaacattcatgtacccactatcaggttaagaaatagaacatttattaatatgtaggaatgttaagaaataaaacatttaataagatctcagaagactccagtaaatctgcaattgtatctctctcctttttaaatgtaaatatcatcttgacttgttaattattcccttgcatttcttttagtttactgccaacacatatattcttcaacaatatatttaattttgaaaaacctgaaaaaaaaaacctgttagcaagtataaaggggcagtattactattattgcatgaaggcttcaagggaaacgttacagtctttgggtcatagtctggcttcagcttcctctgagagtttacagaggccaattttgagcaaattcatggctaaggttatgagtgagttctgctaaacagaaggctcaccacaaggtatctggcaggattatactgggtagctggatgttgcagaaatgtggttagaggaagtaaactgttttttgatgctcacagcatgatgaatcaaactctgtatcttaggattaggttaaaacaatacctttggtatgatatgagtgttgttgctgatccatgcagcatggattggaaagctggggtataagcacacatgctaaagaaaaacatgtaatttggtccatactcacctggatatactgttcctcaggttaaaaaatacagtactatcctaaatcttgaaggcaactctcagcctatccattgagttaccttcagatctgccctctggttcctagctgtcttgggactaacttctttcctgcgctcagctgttttctggattccatgttttccattttattgagtactaacttgttttgctgcagcacatcctttggtagcttctagaggaagtttgtgtggaggtaaaatttttgagaccttgcatgtctcatgtttgattgatactttatacgtttaggtaggaggtaattttccttcaggactttaaaaatattgttgctccattttctttgtttctattgttgtattgagaaatccaatgccattttgatttccccatcataaatttcatgatgatgtgtcttggtgtgggtctatatttatccattgtattgggttttaggtgaacccttccagatagtaactcatttctgtcagttctgggaaacacttagcattggttgatgatttattctctgctgctttgttctcccaactattatttggatgttggatatccagcactgggtatctattttcttacctccctcccttgaccccagtctctgttttttagctctttagctcaatcttccaactctttgctattgtattttaaaatcttaagaccccttcttgatttgtagaagttccttttcttacaaccaaaaagcctttatctatggatttgttcacagataaggggtattcaatatagtgtatttttttttcatttaaaattgtttgcgcatctatttcctccaaatttctttctgtatttattttttgttgtctatatttcagacttttccaggatatctgataatctttggctgtcttcttatggttgaaagagggactaaaaagcttggaaagcctttgggttgtgggaaggggctgtctttaggattatctgaatgggcttttttgggagtcccctcctccacatgaatattttggttttgtcagattccctagaatagaggcttccaatctccttcctggaggggtctgtccaggaaggagattgtctaggggtctgtcagacagcagctttcagctacttccttgatctttttcactaatgattatatagtcatctaactactgtcaacaagtaatagatatcctatccttcacttgtttagattatttgctgagataacctctcaaaagaacctctcaaaataaaaggttaacaagagcctatatcttatatttttcttctctttatcttgttagaagatagctattaaaacctgttctttttctgtcttgataaacacacttcaatcttggtagaatggtagatgggacagtatattttaggacctaaagctctgcaaatgtatgatcagcttgtaagtacaggtgctcaaaaacatgtaaacaatcatgctttttactctgtaggaatatctttaaaattcttgtgaatttttccccagaagtaaagcaaatcttcccccagaaataaaattaaatgtgcataatctaaagctttttttttttattgtggtaggatatatatataaaacataatttgccattgtaaacattttaaatttacaagtcagaggcattaattacatcacaatgttgtgaaattattactactatttccaaaattttctcatcaccccaaactgaaactctgtaactgttgagcaataacctcattcctgtatctctcccaaccccaggtaacctcaaatctttctttttatctttgagacaaggtctcattctatcactcaggtaggagtgcagtggtgtgatcatagctcattgcagcctcaaaatcctgggctcaagcaatcctccttgagtagctaagactataggcacacattaactgcgcctggctgattttgttttttgtagagatgtggtcttgctatgtttcccatgctggtcttgagttcctggcctcaagcagtccttaagattcatccatgttgtggcatgtgtcagaatttcatttgtttttatgactaaataatattccattgtatgtatatacattttgttcatccatcttctgatgaacactgggatatgtctaccttttggctattgtgaataatgctgcagtaaacattgacataacaagtatgtatttgattgcctgtttctaagttcttttgggtatacatcttgagtagaattgctagataatgtcatgttttatttctcttgtgatttcttcttcgatcccctggttgagtgtgttaatttctacatgtttatgaatttcccactgtttttttgttattgatttccaagttcattccattgtgattagagaagatacttagtatgattttaatgtttttgagaattggtgtgtggcctgatagatggtctgtcctggagaatgttcctcatacacttgagcaaaatatttatcatgctattgttgactgtagttttctatatgtctcttaggtcaaggtggtttacaatgtgttaaggttctctttttttaaaaaaatttttgcacagagtatctttttctatgtgttccatgtatttgtgtctttggagctatagtctcttgtagacagcatatcactatcttgttttgttttgttttttctgtccattctgccaatttctgccttttgattggaaaatttaatccatttgcatttaaagtaattaaggaaggactttcttctaccatttaacacttcttctatatgtcatatacttttttggcccctcatttcctctttatggccttcttttctgtttttttgtagtgaactagtctgattctctttccactcccctttgtgtatatttgttagatgttttatttgtggttgctatggggattatagttaacatcctacacttaaaacaatctaatttaaactgataccaatttaccttcaatagcatacaaaatctctactcctgtaaagctctgcccctgccccccttatgttattgatggcacaaattgcctaataaataatttatagttatttgtatgagtttgtcttttaaatcatttaggaaataaaaagtggagttagaaaacagtatgatagtaatactgacttttatatttgtcaatatatttatcttattttggatccttatttcattatatagatttgagttactgtctagtgcccttccatttcggcccaaaggattcccttatgcatttcttgcagggcaagtctaattgtaataaactccctcagcttttgttttatctgagaatgtcttgatttctcccttatttttgatggataattttgccagatacatgaatttttggtaacagtatttttctttcagcactttaaatatgtcatcccactaccttctgacttcatggtttctcatgagatattagatgttataaaatttgaggattcctcattcttgatgagtcagttctgtcttattgcttttcggatttgctcagcttttgtcttttgacagtttgattataacgcggctcagtgtgggtctctgagtttatcccacttagagtttgttgagtttcttggagtcatagatttatgtcttttatcaaattttggacatatttggctattatttcttcaatttttttcactgcttctttcttttccttctgaaatattcttaatgtatatgttggtctgtttgatgctgtctcaccagtttcttaggctgtgttctcttttgttcctcagacttgattattgcagttgcccttctttttatttttttcaagtttgttgattcttctccctgttcagatcaactgttgaactcctctagtgaatttatttcagttactgtacttttcagctccaagatttatctttggttcctttttataacgtctgtgtctttattgatattctcattttgttcatatgtctctttcttcctttagttctttgtccatgttttcctttagctctttgggcttatttaagacaattgtttaaagtctttgcatagtaagtccaatgtctgtgtttcttcagggatggttttcattattttgttttcaatgagccatactttcctgtgtctttgtatgctgtctttttgttgttgaaaactgtatgtttgaacatcataacgtggtggccctgaaaatcagatattccccccttcctgagagttagttttatttttattattgaagattgtagcagtctattgctacatgtgcagtcatttccaaactatttttgcaaagactgtattccttctgtgtgtcatcactgaagtctctgttccttagtttgtgtttaatagtttgacatagatttccttgaaaggagttaaaactagcagaaaaatctctctcccagtctttccagtctttgtagattggttctgtgctgggcttttccattaatacttagccaggcttgtactgagcctaacaatcaggcccaaaagcgtagggtctttgcagatcttgtctgagcatgcttcttgctgtgtatgcacgtagttttctaaatctccctgtatgtgctgttgaatattctaatttcccaaagaaactcctttgcagctttttctcacagaacatagatggttttttggatatcttgaccatagtctttcgacccaggtgtttgcggttgttagttcaccttacacttttttcaagcattgcctactgcttacgatgagtgctctgtcaatcctttaagtagccccagacaggctaccagagacttaaacaagaatttgtaagttctgctcagcttcctctagaaatggggatcagggtccaagacagaatgcagttgctgatttcaagactgctgcaacaccagggagcttgtgggggaagggcaagcagaaatgtcacaaagctttcttgccattttaaagttgcctgttcttgactcagcatttgcttcattgctataaactttttactgtttttcagagttctgataaaattggctatgcctgttcctgctttaaaaaatatatatatattttttagggattggggtctcactatactgaccaggctggtcttgaacttctggcctcaagccatcctctcatttcagcttcccaaagtgctgcaattacacgcgtgaaccaccacacccagcccctgcttgtttttcaatgtgcctactccaccatgttgctcaagtatgtatattttctaaactaccttgtagtgttgtgatgggaaataaatccctgagccttttgaataactcagagagatcaaaaacttagtttatcctattcgaaggattagaaaaatgatatatctttcactttttcagggataggctcctcattagaaggctcctatgtgccgatgctgtacaagacatttcatttctcttaatgtttacaacaagcttgttgccaaggctgatcttgaactcctggcctcaaacgatcctcccagctcagtctcacaaagtgttgggatgtctggccaactaatgactatcttaactcttgtgtttcaatgtttatgccttcttttatcttgactgattgtatgactatgtcttctagaacaatgttgaacagaaatggtgagagcagacatccttgctttaatatttcaccattatatatgatgttaggtatagatttttctcacagatgccttttatcagattgaggaatttatattcctactttgccgaaaggtttttgtagtatgagggggtgctgaattttgtcaaacactttttcggtaataattgagatgattggttctgcagtcatcgagatgtggattttctcctttattctgttcgtgagtgattacactggttgactaatgttaaaacaaccttactttccaggaataaaccctattatcttttttataca

SEQ ID NO: 38 - DNA, 1821 bp, Homo sapiens
  source 1..1821: mol_type = other DNA
tgcgggtacggacagcgcatgagcttatgttgagggcggagcccagaccagcccttcgtcctatcctgcccttccagcacctctcagccgtaacttaaactacacttcccagaagcctcctcagccagggacttccgttgtcgtcagcggaagcggtgacagatcatcccaggccacacagaggccggcttggtcactatggaggagataggcatcttggtggagaaggctcaggatgagatcccagcactgtccgtgtcccggccccagaccggcctgtccttcctgggccctgagcctgaggacctggaggacctgtacagccgctacaaggaggaggtgaagcgaatccaaagcatcccgctggtcatcggacaatttctggaggctgtggatcagaatacagccatcgtgggctctaccacaggctccaactattatgtgcgcatcctgagcaccatcgatcgggagctgctcaagcccaacgcctcagtggccctccacaagcacagcaatgcactggtggacgtgctgccccccgaagccgacagcagcatcatgatgctcacctcagaccagaagccagatgtgatgtacgcggacatcggaggcatggacatccagaagcaggaggtgcgggaggccgtggagctcccgctcacgcatttcgagctctacaagcagatcggcatcgatcccccccgaggcgtcctcatgtatggcccacctggctgtgggaagaccatgttggcaaaggcggtggcacatcacacaacagctgcattcatccgggtcgtgggctcggagtttgtacagaagtatctgggtgagggcccccgcatggtccgggatgtgttccgcctggccaaggagaatgcacctgccatcatcttcatagacgagattgatgccatcgccaccaagagattcgatgctcagacaggggccgacagggaggttcagaggatcctgctggagctgctgaatcagatggatggatttgatcagaatgtcaatgtcaaggtaatcatggccacaaacagagcagacaccctggatccggccctgctacggccaggacggctggaccgtaaaattgaatttccacttcctgaccgccgccagaagagattgattttctccactatcactagcaagatgaacctctctgaggaggttgacttggaagactatgtggcccggccagataagatttcaggagctgatattaactccatctgtcaggagagtggaatgttggctgtccgtgaaaaccgctacattgtcctggccaaggacttcgagaaagcatacaagactgtcatcaagaaggacgagcaggagcatgagttttacaagtgacccttcccttccctccaccacaccactcaggggctggggcttctctcgcacccccagcacctctgtcccaaaacctcattcccttttttctttacccaggattggtttcttcaataaatagataagatcgaatccatttaatttcttcttagaagtttaactcctttggagaatgtgggccttgaataggatcctctgggtccctcttaatctgacagatgagcagacgaggtgcatggcctgggttgcagcttgagagaaccaaaatattcaaaccagatgacttccaaaatgtggggaaagggatggaaaatgaacctgagatggagtccttaatcacgggataaagccctgtgcatctccctcatttcctacaggtaaaagacagtaaagaaattcaggtcacaggccttgggagttcataggaaggagatgtccagtgctgtccagtagaacttt

SEQ ID NO: 39 - DNA, 5159 bp, Homo sapiens
  source 1..5159: mol_type = other DNA
ggtcccggaagtgcgccagtcgtaccttcgcggccgcaactcgctcggccgccgccatcttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattcaaattgtcctgcagaggttcattcccctgaccctttccccacattggtaagagtagctgggttttctaagccactctctggaatctctttgtgttagggtctcgatttgaggacattcatttcttcagcagcccattagcaactgagagcccagggatgtcctacaggatagtttcatagtgacaggtggcacttggctaatagaatatggctgatattgtcattaatcattttgtaccttgacatgggttgtctaataaaactcggacccttcttgtgaaatcagttaaataagacttgtctcggtcacctgtgccctgtccagactcgaggcagtggtaacactgcacagtgctatgtggcttctctttgaggatttttgggttttgtaactaaattcttgctgccctcatactttttatgtattagaatcatattcgtattgcccttttaaaacattgggatcctccaaaggcctgccccatgtatttaacagtaatacaggaagcatggcaggcaccatgcaaaccaaggatggatggtgcagtccctgtgtcagtgggcggtggtttcctgctggcctggaatcactcatcacctgattgattggctctgtggtcctgggcaggtgcctcataggtgtgtggatatgatgacgtttctttaaaatgtatgtatttaacaaatacttaattgtattaaggtcatgtaccaaggatttgataaagtttaaataatttactctctacttttatccattttatccattttaactcatgtaatcctcatgtgagtattcctgtttaacacttgagtaaactgaggcacagagaacataagttgcatgccatagtcacacactgtgaaagtgaaaagagaatgtgtgcaaaacacgtcacagtcctggtttctgagtaaaggcaggctgttatctttagaatcaagctatcacagggagataggcaatgctgtgggtgttggaggaaggtgagagcctgttgctaacaatttcctggttttaaagctaaggctgattttattgggaagatctcacatgtgtgtggcccctgagagttcccagtgccttttatttgcagtccttccatttggacctcctagctgccccatcaggtcatctccagggctcagaggggtgagaccatttcccaaggtcacagaaccagctctctagtcaccaccctgcctctccctctcacccagagtcagtaccagttttatggctttattacaaactgctgggtccctcccattttcaacttgattgatgggatgtcatcccttatcctgtctgacatttgcctctggcctggttgctagaagtttgccccaggggcaagagttgaaatttggcttcctgaggtgggctttgtggtttgcgtccctaaagtgagcccactactggttgcttgtccatggccaacaccagaaatcccctgagcactacctgggtctcattccaagaaggaagagggtcaggagacctggggagtctcatattccaagttcttctttctttctgggagcagtgggcagttcatggtgttagggcactcacccccacagactggcaaaccctgcaggacttccgtggctgaggctgtgaccggaggccaggaatgccgttgggtggattgtgagtgaatgggccctttgagctgccctctagagagcaaatccagtttcctggagctcctgaatgaatatctgtactggctcgctcagatgcagaagctccattgaccatgaggccttgtgaacatcagtggccacaggcccagtgtgctgcttggcactgcactagtttaggacctgcagcatgtaggtagcgtcctagtgtttataatacaaagctgctctgcacagcttttctgattcttcttgcaatctcctgaggattatctgccccatttttaaaacgaggtggaatacccaaggtcatgtagccagtgagtgctctggaaagccaaagcagctcatcccttcctggggaccacactgctctgctccaccagaccacactatgaaataggaataagtgctcctgttgcaggactgctgggaaaacaggtggtgtgggacttaagtcaccataattttgaagacttgcatgcagagggctccaggaattgtagacattaaggaatttcactttcagttctacccactacttaagtacttgtcatgtactcttagaggaggccagtaatgatcagaaccattttactttaaaattaataatattgtattagagaatatattaaatggttatattgggttatgttaggatatatacttgaatggaaatacatgtactattagcaatcatatttcatttatccctgtaattagacaagaaagcataatatagctctactcatgggtacacataccagtgtataagatttttagaagtttactttttaaaaataaaagcaaaatgtaagatcttaaaaaaaaaaaaaaaaaa

SEQ ID NO: 40 - DNA, 5416 bp, Homo sapiens
  source 1..5416: mol_type = other DNA
agtgggccgccatgttgtcggagtgaaaggtaagggggagcgagagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaatggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcaggtaattaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctgaaaaaaaaaaaaaaaaaaaaaaaaaaaaaaa

SEQ ID NO: 41 - DNA, 1940 bp, Homo sapiens
  source 1..1940: mol_type = other DNA
gagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaggaaaaaaaaaaaaaaaaaa

SEQ ID NO: 42 - DNA, 1309 bp, Homo sapiens
  source 1..1309: mol_type = other DNA
gctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttaaaaaaaaaaaaaaaaaaaaa

SEQ ID NO: 43 - DNA, 2321 bp, Homo sapiens
  source 1..2321: mol_type = other DNA
gtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaaaaaaaaaaaaaaaaaaaaaaa

SEQ ID NO: 44 - DNA, 1229 bp, Homo sapiens
  source 1..1229: mol_type = other DNA
gtctgacgggcgatggcgcagccaatagacaggagcgctatccgcggtttctgattggctactttgttcgcattataaaaggcacgcgcgggcgcgaggcccttctctcgccaggcgtcctcgtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 45 - DNA, 5234 bp, Homo sapiens
  source 1..5234: mol_type = other DNA
agagcgtcgggatatcgggtggcggctcgggacggaggacgcgctagtgtgagtgcgggcttctagaactacaccgaccctcgtgtcctcccttcatcctgcggggctggctggagcggccgctccggtgctgtccagcagccatagggagccgcacggggagcgggaaagcggtcgcggccccaggcggggcggccgggatggagcggggccgcgagcctgtggggaaggggctgtggcggcgcctcgagcggctgcaggttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccttgtctgatttaataaaatacttaaacactgaaaaaaaaaaa

SEQ ID NO: 46 - DNA, 1869 bp, Homo sapiens
  source 1..1869: mol_type = other DNA
tacctggttgatcctgccagtagcatatgcttgtctcaaagattaagccatgcatgtctaagtacgcacggccggtacagtgaaactgcgaatggctcattaaatcagttatggttcctttggtcgctcgctcctctcccacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 47 - DNA, 2288 bp, Homo sapiens
  source 1..2288: mol_type = other DNA
ggggccgaacgtggtataaaaggggcgggaggccaggctcgtgccgttttgcagacgccaccgccgaggaaaaccgtgtactattagccatggtcaaccccaccgtgttcttcgacattgccgtcgacggcgagcccttgggccgcgtctcctttgagctgtttgcagacaaggtcccaaagacagcagaaaattttcgtgctctgagcactggagagaaaggatttggttataagggttcctgctttcacagaattattccagggtttatgtgtcagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggttggatggcaagcatgtggtgtttggcaaagtgaaagaaggcatgaatattgtggaggccatggagcgctttgggtccaggaatggcaagaccagcaagaagatcaccattgctgactgtggacaactcgaataagtttgacttgtgttttatcttaaccaccagatcattccttctgtagctcaggagagcacccctccaccccatttgctcgcagtatcctagaatctttgtgctctcgctgcagttccctttgggttccatgttttccttgttccctcccatgcctagctggattgcagagttaagtttatgattatgaaataaaaactaaataacaattgtcctcgtttgagttaagagtgttgatgtaggctttattttaagcagtaatgggttacttctgaaacatcacttgtttgcttaattctacacagtacttagattttttttactttccagtcccaggaagtgtcaatgtttgttgagtggaatattgaaaatgtaggcagcaactgggcatggtggctcactgtctgtaatgtattacctgaggcagaagaccacctgagggtaggagtcaagatcagcctgggcaacatagtgagacgctgtctctacaaaaaataattagcctggcctggtggtgcatgcctagtcctagctgatctggaggctgacgtgggaggattgcttgagcctagagtgagctattatcatgccactgtacagcctgggtgttcacagatcttgtgtctcaaaggtaggcagaggcaggaaaagcaaggagccagaattaagaggttgggtcagtctgcagtgagttcatgcatttagaggtgttcttcaagatgactaatgtcaaaaattgagacatctgttgcggttttttttttttttttttcccctggaatgcagtggcgtgatctcagctcactgcagcctccgcctcctgggttcaagtgattctagtgcctcagcctcctgagtagctgggataatgggcgtgtgccaccatgcccagctaatttttgtatttttagtatagatggggtttcatcattttgaccaggctggtctcaaactcttgacctcagctgatgcgcctgccttggcctcccaaactgctgagattacagatgtgagccaccgcaccctacctcattttctgtaacaaagctaagcttgaacactgttgatgttcttgagggaagcatattgggctttaggctgtaggtcaagtttatacatcttaattatggtggaattcctatgtagagtctaaaaagccaggtacttggtgctacagtcagtctccctgcagagggttaaggcgcagactacctgcagtgaggaggtactgcttgtagcatatagagcctctccctagctttggttatggaggctttgaggttttgcaaacctgaccaatttaagccataagatctggtcaaagggatacccttcccactaaggacttggtttctcaggaaattatatgtacagtgcttgctggcagttagatgtcaggacaatctaagctgagaaaaccccttctctgcccaccttaacagacctctagggttcttaacccagcaatcaagtttgcctatcctagaggtggcggatttgatcatttggtgtgttgggcaatttttgttttactgtctggttccttctgcgtgaattaccaccaccaccacttgtgcatctcagtcttgtgtgttgtctggttacgtattccctgggtgataccattcaatgtcttaatgtacttgtggctcagacctgagtgcaaggtggaaataaacatcaaacatcttttcattatcccta

SEQ ID NO: 48 - DNA, 2439 bp, Homo sapiens
  source 1..2439: mol_type = other DNA
gagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcttttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaa

SEQ ID NO: 49 - DNA, 1196 bp, Homo sapiens
  source 1..1196: mol_type = other DNA
cacttctgccgcccctgtttcaagggataagaaaccctgcgacaaaacctcctccttttccaagcggctgccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 50 - DNA, 987 bp, Homo sapiens
  source 1..987: mol_type = other DNA
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 51 - DNA, 3003 bp, Homo sapiens
  source 1..3003: mol_type = other DNA
ctttctccttccccttcttccgggctcccgtcccggctcatcacccggcctgtggcccactcccaccgccagctggaaccctggggactacgacgtccctcaaaccttgcttctaggagataaaaagaacatccagtcatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggtttgtcctggcggggaataaaagggatcattggttccagtcacaggtgtagtaattgtgggtactttaaggtttggagcacttacaaggctgtggtagaatcataccccatggataccacatattaaaccatgtatatctgtggaatactcaatgtgtacacctttgactacagctgcagaagtgttcctttagacaaagttgtgacccattttactctggataagggcagaaacggttcacattccattatttgtaaagttacctgctgttagctttcattatttttgctacactcattttatttgtatttaaatgttttaggcaacctaagaacaaatgtaaaagtaaagatgcaggaaaaatgaattgcttggtattcattacttcatgtatatcaagcacagcagtaaaacaaaaacccatgtatttaacttttttttaggatttttgcttttgtgatttttttttttttgatacttgcctaacatgcatgtgctgtaaaaatagttaacagggaaataacttgagatgatggctagctttgtttaatgtcttatgaaattttcatgaacaatccaagcataattgttaagaacacgtgtattaaattcatgtaagtggaataaaagttttatgaatggacttttcaactactttctctacagcttttcatgtaaattagtcttggttctgaaacttctctaaaggaaattgtacattttttgaaatttattccttattccctcttggcagctaatgggctcttaccaagtttaaacacaaaatttatcataacaaaaatactactaatataactactgtttccatgtcccatgatcccctctcttcctccccaccctgaaaaaaatgagttcctattttttctgggagagggggggattgattagaaaaaaatgtagtgtgttccatttaaaattttggcatatggcattttctaacttaggaagccacaatgttcttggcccatcatgacattgggtagcattaactgtaagttttgtgcttccaaatcactttttggtttttaagaatttcttgatactcttatagcctgccttcaattttgatcctttattctttctatttgtcaggtgcacaagattaccttcctgttttagccttctgtcttgtcaccaaccattcttacttggtggccatgtacttggaaaaaggccgcatgatctttctggctccactcagtgtctaaggcaccctgcttcctttgcttgcatcccacagactatttccctcatcctatttactgcagcaaatctctccttagttgatgagactgtgtttatctccctttaaaaccctacctatcctgaatggtctgtcattgtctgcctttaaaatccttcctctttcttcctcctctattctctaaataatgatggggctaagttatacccaaagctcactttacaaaatatttcctcagtactttgcagaaaacaccaaacaaaaatgccattttaaaaaaggtgtattttttcttttagaatgtaagctcctcaagagcagggacaatgttttctgtatgttctattgtgcctagtacactgtaaatgctcaataaatattgatgatgggaggcagtgagtcttgatgataagggtgagaaactgaaatcccaaacactgttttgttgcttgttttattatgacctcagattaaattgggaaatattggcccttttgaataattgtcccaaatattacattcaaataaaagtgcaatggagaaaaaaaaaaa

SEQ ID NO: 52 - DNA, 2803 bp, Homo sapiens
  source 1..2803: mol_type = other DNA
actgcagccccgctcgactccggcgtggtgcgcaggcgcggtatcccccctcccccgccagctcgaccccggtgtggtgcgcaggcgcagtctgcgcagggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaatccatttgaaatattttactgttgtgactttagtcatatttgttgacctaaaaatcaaatgtaatctttgtattgtgttacatcaaaatccagatattttgtatagtttcttttttctttttcttttcttttttttttttgagacaggatcggtgcagtagtacaatcacagctcactgcagcctcaaactcctgggcagctcaggtgatcttcctgactcagccttctgagtagttggggctacaggtgtgcaccaccatgcccagctcatttattttgtaattgtagggacagggtctcactgtgttgcctaggctggtctcaagtgatcctccctccttggcctcccaaggtgctggaattataggtgtgaacaaaccaaaaaaaaaaaaaa

SEQ ID NO: 53 - DNA, 1435 bp, Homo sapiens
  source 1..1435: mol_type = other DNA
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 54 - DNA, 4476 bp, Homo sapiens
  source 1..4476: mol_type = other DNA
agcagagagaacacacgtccttgcggaagtgacggcagttccgagtccagtgggggcggtgggagcgatgagggtctgagacggtgggagcggttgtgtgaagatggagacattccatacaccaagcttgggtgatgaggaatttgaaatcccacctatctccttggattctgatccctcattggctgtctcagatgtggttggccactttgatgacctggcagacccttcctcttcacaggatggcagtttttcagcccagtatggggtccagacattggacatgcctgtgggcatgacccatggcttgatggagcagggcggggggctcctgagtgggggcttgaccatggacttggaccactctataggaactcagtatagtgccaacccacctgttacaattgatgtaccaatgacagacatgacatctggcttgatggggcatagccagttgaccaccattgatcagtcagaactgagttcccagctgggtttgagcctagggggtggcaccatcctgccacctgcccagtcacctgaagatcgtctttcaaccaccccttcacctactagttcacttcacgaggatggtgttgaggatttccggaggcaacttcccagccagaagacagtcgtggtggaagcagggaaaaagcagaaggccccaaagaagagaaaaaagaaagatcctaatgaacctcagaaaccagtttcagcatatgctttattctttcgtgatacacaggctgccatcaagggacagaatcctaatgccacttttggtgaggtttcaaaaattgtggcctccatgtgggatagtcttggagaggagcaaaaacaggtatataagaggaaaactgaggctgccaagaaagagtatctgaaggcactggctgcttacaaagacaaccaggagtgtcaggccactgtggaaacagtggaattggatccagcaccaccatcacaaactccttctccacctcctatggctactgttgacccagcatctccagcaccagcttcaatagagccccctgccctgtccccatccattgttgttaactccaccctttcatcctatgtggcaaaccaggcatcttctggagctgggggtcagcccaatatcaccaagttgattattaccaaacaaatgttgccctcttctattactatgtctcaaggagggatggttactgttatcccagccacagtggtgacctcccgggggctccaactaggccaaaccagtacagctactatccagcccagtcaacaagcccagattgtcactcggtcagtgttgcaggcagcagcagctgctgctgctgctgcttctatgcaactgcctccaccccgactacagccccctccattacaacagatgccacagcccccgactcagcagcaagttaccattctgcagcagcctcctccactccaggccatgcaacagcctccacctcagaaagttcgaatcaatttacagcaacagcctcctcctctgcagatcaagagtgtgcctctacccactttgaaaatgcagactaccttagtcccaccaactgtggaaagtagtcctgagcggcctatgaacaacagccctgaggcccatacagtggaggcaccttctcctgagactatctgtgagatgatcacagatgtagttcctgaggttgagtctccttctcagatggatgttgaattggtgagtgggtctcctgtggcactctcaccccagcctcgatgtgtgaggtctggttgtgagaaccctcccattgtgagtaaggactgggacaatgaatactgcagcaatgagtgtgtggtgaagcactgcagggatgtattcttggcctgggtagcctctagaaattcaaacacagtggtgtttgtgaaatagtccttcctgttctccaagccagtgaagagttatctgctgggaaagtgtccaagagcctgtttttgaaacacaagctgggcttctggtagtgcctcatcacaacccatgatggctgttcatgtttcaccccttttcttccttcagcagaggccaggctatggagcagggccactgaatttgctgtaatctggagatgctttttactttcaaccataagcggtaatagcagaggaaagggtgaagggagtctgggcaagcaaagcatagagatggtggggtggtggtggggttgaagaaacttgttggtataattgtcataggacttgcctaaaatattattaaaattacgggagtgtactcagctttgagcctaggagaaaatgccactgtgtgcatccattttaaagggttccctcataaaaaaatgttattccccattatcacatcagtacactgctttgaaaacaaaacttttcaacatgggcatactgggctacatggaaaatgacatcacccaggagtgatttctctttatatatattatttctgcagttaccatccttatctgagttatcacagttcatgaatctaagaggcggaactctacatcattagtaagaggttccaccaaagtctaaagttgtattcacttgtgtttgatgaactatctttaaaagaccataggtctatcattatttcttagacataatctaaagaaaaacagactagagaagccacctggttgtaacagaataagcagaagtttacagcatgatagtccaagtggtgataactttaaataaaactcaaatttttactgtttgtagacaggaatgctgtcctagagaacctcctcctcaaccagctacgtacatagttttatcctatgcattcctgttttctgtgtgttttttgttttttttttttttttttttttttgagacagagtctcgctctgtcacccaggctggagtgcagtggtgcgacctcagctcactgaaacctctgcctcccgggttcaagcgattctcctgcatcagcctcccgagtagctaggattacaggcgcccgccactacgcccagctaatttgtggtatttttagtagagacagggtttcaccatgttggccaggctggtctcgaactcctgacctcatgatccgcccgccttgacctcccaaagtgctgggattacaggcatgagccaccgcacccagcctgcattcctgtttttttaatggttttggagggtagcagtagagatggggtctcactatgttgcccagtctagtcttgaactcctgggctacagttaccctcctacctcggcttcccaaagtgctcggattacaggtgtgagccactgtgcctagcctataatgatcattttaatgtttcccatgcactcatttagtttgaaccttcacagcaacccaatgaggtaatactcccatttcacatataatactgagagatgagttgcacaagattatacactgttaagtagcagagccagaatggacttcagaatcccaactacaatacaaatgtttatttaaataaagaagaaagctattgtacaaatatcactcttcaggtttagcttacagagccatggctatggattcttagctctgtaaggaagtgcttctataaattcttaggtttagagatgataccatctgggtacctttgcttgaaccgtgcaaccacatctgggtctagtaggtggatcccatccagttggtttccaagggtgatcctgaaacagtgtaaaaggaggggcaaaccagaaatcctggaattagagggtttaatattgttaaaaaatgcataccaaatgaagactgcctatcatcatatcaaatatgccaattctaaaaagagcttaacattagaatagtatatggtagaattactagttcagaattggcatagattctggtgttaaaatagactggatctgtattatctgagggttagtaactaatgcttagccaggcctgcttcacagagttgctaccagggagtattctttggataagcaaaatgctagcagcatgtgttttaagctctgttaaggggtgaaagatgtaattattgacagattaaatagataacttcgtaaccaccagggggcagattcaatacatcacagaatggctgaggaagatccttgggttgtgaagagagtagaaaccctagggagcagtgcttttgggtcctagaacctgttgagtttctaatgaatatttgtagaatctcataaaacagtttaaatacaagcttaagtggcttatgaatcctgtgaagctcatttatggactagtgtaaaacaatgtgaagctctactaagttctgtccttaatcataaataatagccccttgaggactagcctgttctctggtcaccttaccagttgggttgcacattgtgtggtcgtccaaataactcaatcttgcgagtgccaggagatagtctttcaatcatgccatagatttcatctggtttatgactggtggaacgaacctaggaaataaaaactagctgctttttaagttacacaagaaaaaa